(19) United States
(12) Patent Application Publication
(10) Pub. No.: US 2026/0364089 A1
(43) Pub. Date: Nov. 19, 2026

(54) PYRIDOPYRIMIDINE KRAS INHIBITORS

(71) Applicant: Treeline Biosciences, Inc., Watertown, MA (US)

(72) Inventors: James Paul Lajiness, San Diego, CA (US); Ryan L. Gianatassio, San Diego, CA (US); Tami J. Marrone, San Diego, CA (US); Kyle Stephen McClymont, San Diego, CA (US); Andrew Valiere, San Diego, CA (US)

(21) Appl. No.: 19/103,922

(22) PCT Filed: Aug. 16, 2023
(86) PCT No.: PCT/US2023/072332

§ 371 Date: Feb. 14, 2025

Related U.S. Application Data
(60) Provisional application No. 63/398,737, filed on Aug. 17, 2022.
(60) Provisional application No. 63/426,885, filed on Nov. 21, 2022.
(60) Provisional application No. 63/456,316, filed on Mar. 31, 2023.
(60) Provisional application No. 63/526,503, filed on Jul. 13, 2023.

(51) Int. Cl. A61K 31/553; A61K 31/529; A61K 31/5377; C07D 519/00

(52) U.S. Cl. CPC A61K 31/553; A61K 31/529; A61K 31/5377; C07D 519/00

(57) ABSTRACT

This disclosure provides compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, that inhibit a KRas protein. In some embodiments, the KRas protein has a dysregulation (e.g., the KRas protein is mutated or amplified). These compounds are useful, for example, for treating a disease, disorder, or condition in which increased and/or sustained (e.g., excessive) KRas activation, for example, KRas activation associated with a mutant KRas protein, contributes to the pathology and/or symptoms and/or progression of the disease, disorder, or condition (e.g., cancer) in a subject (e.g., a human). This disclosure also provides compositions containing the compounds provided herein, or pharmaceutically acceptable salts thereof, as well as methods of using and making the same.

### CROSS-REFERENCE TO RELATED APPLICATIONS

This application claims priority to U.S. Provisional Application Ser. No. 63/398,737, filed on Aug. 17, 2022; 63/426,885, filed on Nov. 21, 2022; 63/456,316, filed on Mar. 31, 2023; and 63/526,503, filed on Jul. 13, 2023; each of which is incorporated by reference in its entirety herein.

### DESCRIPTION OF THE TEXT FILE SUBMITTED ELECTRONICALLY

This application contains a Sequence Listing which has been submitted electronically in XML format. The Sequence Listing XML is incorporated herein by reference. Said XML file, created on Aug. 14, 2023, is named TRLN-008-004W01_ST26_SL.xml and is 2,059 bytes in size.

### TECHNICAL FIELD

This disclosure provides compounds of Formula (A) (e.g., Formula (I) (e.g., Formula (I-a), (I-aa), (I-b), (I-bb), (I-c), (I-cc), (I-d), (I-dd), (I-e), (I-eel), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, that inhibit a KRas GTPase (e.g., a KRas GTPase that has a dysregulation (referred to herein as a dysregulated KRas protein)). In some embodiments, the KRas protein is a dysregulated KRas protein that has a mutation (referred to herein as a mutant KRas protein). These compounds are useful, for example, for treating a disease, disorder, or condition in which increased and/or sustained (e.g., excessive) KRas activation, such as KRas activation associated with a mutant KRas protein, contributes to the pathology and/or symptoms and/or progression of the disease, disorder, or condition (e.g., cancer) in a subject (e.g., a human). This disclosure also provides compositions containing compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, as well as methods of using and making the same.

### BACKGROUND

The KRAS gene is frequently dysregulated (e.g., mutated or amplified) in various human cancers. Oncogenic mutations in KRas typically occur at hotspots in the protein such as at amino acids positions 12, 13, and 61. In some cases, a mutation can lead to maintenance of KRas activation (GTP-bound state), for example, due to a deficiency of intrinsic GTPase activity and/or insensitivity for GTPase-activating proteins (GAPs) and consequent increased KRas signaling. Specifically, some of the most common protein mutations include those at position 12 (referred to herein as G12X) such as G12A, G12C, G12D, G12R, G12S, and G12V; position 13 (referred to herein as G13X) such as G13C, G13D, and G13V; and Q61 (referred to herein as Q61X), such as Q61E, Q61H, Q61K, Q61L, Q61P, and Q61R.

KRas is widely recognized as a target for the design and development of therapies that can specifically bind and inhibit KRas signaling in cancer cells but had long been considered to be undruggable. Currently, there are few approved KRas-targeted therapies.

In some embodiments, the KRas protein is a dysregulated KRas protein that has a mutation (referred to herein as a mutant KRas protein). These compounds are useful, for example, for treating a disease, disorder, or condition in which increased and/or sustained (e.g., excessive) KRas activation, such as KRas activation associated with a mutant KRas protein, contributes to the pathology and/or symptoms and/or progression of the disease, disorder, or condition (e.g., cancer) in a subject (e.g., a human). This disclosure also provides compositions containing the same as well as methods of using and making the same.

### SUMMARY

This disclosure provides compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, that inhibit a KRas protein (e.g., a dysregulated KRas protein, such as a mutant KRas protein). These compounds are useful, for example, for treating a disease, disorder, or condition in which increased KRas activation, such as KRas activation associated with a mutant KRas protein or KRas activation associated with KRas amplification, contributes to the pathology and/or symptoms and/or progression of the disease, disorder, or condition (e.g., cancer) in a subject (e.g., a human). This disclosure also provides compositions containing compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, as well as methods of using and making the same.

Provided herein are compounds of Formula (A):

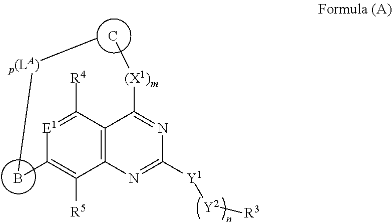

or pharmaceutically acceptable salts thereof, wherein:
m is 0, 1, or 2;
each X¹ is independently selected from the group consisting of CH₂, CHR^{a}, C(R^{a})₂, O, N(R^{d}), C(═O), and S(O)₀₋₂, provided that —(X¹)ₘ— does not include any O—O, O—N, N—S(O)₀, or O—S(O)₀₋₂ bonds;
Ring C is selected from the group consisting of: C₃₋₁₅ cycloalkylene, 4-15 membered heterocyclylene, 6-15 membered arylene, and 5-15 membered heteroarylene, each of which is optionally substituted with 1-4 R⁷, wherein:
each R⁷ is independently selected from the group consisting of R^{a} and R^{b};
p is an integer from 3 to 10;
each L^{A} is independently selected from the group consisting of: L^{A1}, L^{A2}, L^{A3}, and L^{A4}, provided that 0-2 occurrences of L^{A} are L^{A4}, and 0-3 occurrences of L^{A} are selected from the group consisting of L^{A2} and L^{A3};
each L^{A1} is independently selected from the group consisting of: —CH₂—, —CHR^{L}—, and —C(R^{L})₂—, wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, and C₁₋₆ alkyl optionally substituted with 1-6 R^{c};
each L^{A2} is independently selected from the group consisting of:

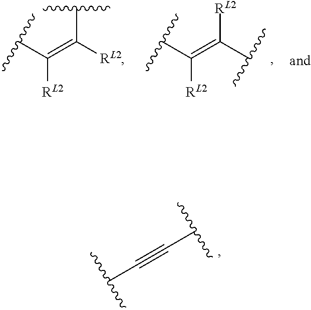

wherein each R^{L2} is independently selected from the group consisting of: H, halo, CN, and C₁₋₆ alkyl optionally substituted with 1-6 R^{c};
each L^{A3} is independently selected from the group consisting of: —N(R^{d})—, —O—, —S(O)₀₋₂—, and C(═O);
each L^{A4} is independently selected from the group consisting of: C₃₋₁₀ cycloalkylene, 4-10 membered heterocyclylene, C₆₋₁₀ arylene, and 5-10 membered heteroarylene, each of which is optionally substituted with 1-3 R^{g};
Ring B is selected from the group consisting of: C₆₋₁₀ arylene and 5-10 membered heteroarylene, each of which is optionally substituted with 1-6 substituents independently selected from the group consisting of: R^{a} and R^{b};
Y¹ is selected from the group consisting of: a bond, —N(H)—, —N(C₁₋₃ alkyl)-, —N(C₃₋₆ cycloalkyl)-, —O—, and —S(O)₀₋₂—;
n is 0 or 1;
Y² is a straight-chain C₁₋₆ alkylene optionally substituted with 1-6 R^{Y};
each R^{Y} is independently selected from the group consisting of: halo, cyano, —OH, oxo, C₁₋₆ alkoxy, C₁₋₆ haloalkoxy, C₁₋₆ alkyl, and C₁₋₆ haloalkyl, or
a pair of R^{Y} on the same or different carbon atom(s) taken together with the atom(s) connecting them form a C₃₋₆ cycloalkyl ring or 4-6 membered heterocyclyl ring, each of which is optionally substituted with 1-3 independently selected C₁₋₃ alkyl;
R³ is selected from the group consisting of:
(a) —H;
(b) -halo;
(c) —NR^{d}R^{e};
(d) C₃₋₁₀ cycloalkyl or 3-15 membered heterocyclyl, each of which is optionally substituted with 1-6 substituents independently selected from the group consisting of: R^{a} and R^{b}; and
(e) C₆₋₁₀ aryl or 5-10 membered heteroaryl, each of which is optionally substituted with 1-6 substituents independently selected from the group consisting of: R^{a} and R^{b},
E¹ is N, CH, or CR⁶;
R⁴, R⁵, and R⁶ are independently selected from the group consisting of:
(a) —H;
(b) halo;
(c) cyano;
(d) C₁₋₆ alkyl;
(e) C₁₋₆ haloalkyl;
(f) C₁₋₆ alkoxy;
(g) C₁₋₆ haloalkoxy;
(h) —(C₀₋₃ alkylene)-C₃₋₆ cycloalkyl, wherein the C₃₋₆ cycloalkyl portion is optionally substituted with 1-3 R^{g}; and
(i) —O—(C₀₋₃ alkylene)-C₃₋₆ cycloalkyl, wherein the C₃₋₆ cycloalkyl portion is optionally substituted with 1-3 R^{g};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
b is 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, cyano, —OH, —C₁₋₆ alkoxy, —C₁₋₆ haloalkoxy, —NH₂, —N(H)(C₁₋₃ alkyl), and —N(C₁₋₃ alkyl)₂.

In some embodiments, the compounds of Formula (A) are compounds of Formula (I):

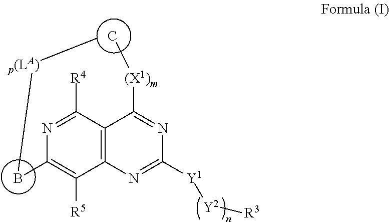

or pharmaceutically acceptable salts thereof.

In some embodiments, the compounds of Formula (A) are compounds of Formula (II):

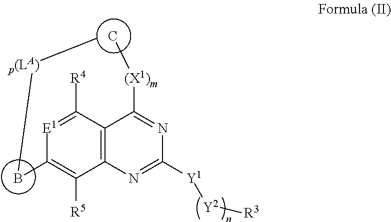

or pharmaceutically acceptable salts thereof, wherein E¹ is CH or CR⁶.

Also provided herein are pharmaceutical compositions comprising a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, and a pharmaceutically acceptable carrier.

Provided herein are methods for treating cancer in a subject in need thereof, the methods comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as provided herein.

Also provided herein are methods for treating cancer in a subject in need thereof, the methods comprising (a) determining that the cancer has a KRas dysregulation (e.g., a KRas mutation (e.g., a KRas G12D mutation, a KRas G12R mutation, or a KRas G12V mutation)); and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as provided herein.

Provided herein are methods of treating a KRas-associated disease or disorder (e.g., a mutant KRas-associated disease or disorder (e.g., a KRas G12D-associated cancer, a KRas G12R-associated cancer, or a KRas G12V-associated cancer)) in a subject, the methods comprising administering to a subject identified or diagnosed as having a KRas-associated disease or disorder a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as provided herein.

This disclosure also provides methods of treating a KRas-associated disease or disorder (e.g., a mutant KRas-associated disease or disorder (e.g., a KRas G12D-associated cancer, a KRas G12R-associated cancer, or a KRas G12V-associated cancer)) in a subject, the methods comprising: determining that the disease or disorder in the subject is a KRas-associated disease or disorder (e.g., a mutant KRas-associated disease or disorder (e.g., a KRas G12D-associated disease or disorder, a KRas G12R-associated disease or disorder, or a KRas G12V-associated disease or disorder)); and administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as provided herein.

Further provided herein are methods of treating a KRas-associated cancer (e.g., a mutant KRas-associated cancer (e.g., a KRas G12D-associated cancer, a KRas G12R-associated cancer, or a KRas G12V-associated cancer)) in a subject, the methods comprising administering to a subject identified or diagnosed as having a KRas-associated cancer (e.g., a mutant KRas-associated cancer (e.g., a KRas G12D-associated cancer, a KRas G12R-associated cancer, or a KRas G12V-associated cancer)) a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as provided herein.

This disclosure also provides methods of treating a KRas-associated cancer (e.g., a mutant KRas-associated cancer (e.g., a KRas G12D-associated cancer, a KRas G12R-associated cancer, or a KRas G12V-associated cancer)) in a subject, the methods comprising: determining that the cancer in the subject has a KRas dysregulation (e.g., a KRas mutation (e.g., a KRas G12D mutation, a KRas G12R mutation, or a KRas G12V mutation)); and administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as provided herein.

To facilitate understanding of the disclosure set forth herein, a number of terms are provided. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties. In the case of conflict between the present disclosure and any content incorporated by reference, the present disclosure controls.

The details of one or more embodiments of the invention are set forth in the accompanying drawings and the description below. Other features and advantages of the invention will be apparent from the description and drawings, and from the claims.

### DETAILED DESCRIPTION

This disclosure provides compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, that inhibit a KRas protein (e.g., a dysregulated KRas protein, such as a mutant KRas protein). These compounds are useful, for example, for treating a disease, disorder, or condition associated with a KRas dysregulation (e.g., a KRas mutation or amplification) in which increased and/or sustained (e.g., excessive) KRas activation contributes to the pathology and/or symptoms and/or progression of the disease, disorder, or condition (e.g., cancer) in a subject (e.g., a human). These compounds can also be useful, for example, for treating a disease, disorder, or condition in which a mutant KRas protein (e.g., a resistance mutation) confers intrinsic resistance to one or more KRas inhibitors (e.g., a KRas inhibitor selective for a KRas G12C mutant protein), or to a non-KRas-targeted therapeutic agent. See, e.g., Misale, et al., Nature 486.7404 (2012): 532-536 and Awad, et al., New England Journal of Medicine 384.25 (2021): 2382-2393. This disclosure also provides compositions containing compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, as provided herein as well as methods of using and making the same.

Ras family genes (e.g., KRAS, NRAS, and HRAS) were the first oncogenes identified and are some of the most commonly mutated of all discovered oncogenes. See, e.g., Hunter et al. Mol Cancer Res. 2015; 13(9):1325-35. The Ras family are guanine nucleotide binding proteins generally found at the inner leaflet of the cell membrane. A wild type Ras protein becomes activated when bound to GTP, but it is inactive when bound to GDP. Normally, growth factors bind to extracellular receptors to induce nucleotide exchange with the help of guanine nucleotide exchange factors (GEF) (e.g., Son of sevenless homolog 1 (SOS1)). These GEFs allow GDP to dissociate from a Ras protein and GTP to bind. Ras proteins can interact with effector proteins such as cRAF when bound to GTP. Hydrolysis of GTP to form GDP can deactivate Ras proteins, and the hydrolysis can be achieved through the intrinsic GTPase activity, which may be enhanced by binding to a GTPase activating protein (GAP). There are 3 major RAS proteins in humans: KRas, HRas, and NRas.

Some oncogenic KRas missense mutations can prevent or slow GTP hydrolysis and result in the accumulation of KRas in the active state. Signaling pathways associated with KRas are persistently activated in many cancers, where they participate in cellular growth and proliferation, differentiation, protein synthesis, glucose metabolism, cell survival, and inflammation.

Mutant KRas proteins often have altered Raf affinity and/or altered intrinsic GTPase activity. See, for example, Table 1 reproduced from Hunter et al. Mol Cancer Res. 2015; 13(9):1325-35. These changes and other factors can contribute to increased KRas signaling in mutant KRas proteins.

TABLE 1

Raf Affinity

| | | High | Low |
|---|---|---|---|
| Intrinsic | Low | G12A | G12R |
| GTPase | | Q61L | G12V |
| activity | High | Wild type | G12D |
| | | G12C | |
| | | G13D | |

### Compound Embodiments

Provided herein are compounds of Formula (A):

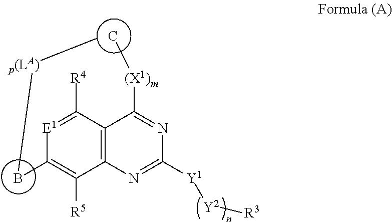

or pharmaceutically acceptable salts thereof, wherein:
m is 0, 1, or 2;
each X¹ is independently selected from the group consisting of CH₂, CHR^{a}, C(R^{a})₂, O, N(R^{d}), C(═O), and S(O)₀₋₂, provided that —(X¹)ₘ— does not include any O—O, O—N, N—S(O)₀, or O—S(O)₀₋₂ bonds;
Ring C is selected from the group consisting of: C₃₋₁₅ cycloalkylene, 4-15 membered heterocyclylene, 6-15 membered arylene, and 5-15 membered heteroarylene, each of which is optionally substituted with 1-4 R⁷, wherein:
each R⁷ is independently selected from the group consisting of R^{a} and R^{b};
p is an integer from 3 to 10;
each L^{A} is independently selected from the group consisting of: L^{A1}, L^{A2}, L^{A3}, and L^{A4}, provided that 0-2 occurrences of L^{A} are L^{A4}, and 0-3 occurrences of L^{A} are selected from the group consisting of L^{A2} and L^{A3};
each L^{A1} is independently selected from the group consisting of: —CH₂—, —CHR^{L}—, and —C(R^{L})₂—, wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, and C₁₋₆ alkyl optionally substituted with 1-6 R^{c};
each L^{A2} is independently selected from the group consisting of:

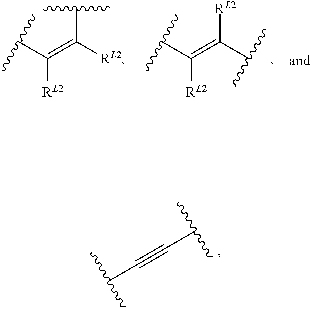

wherein each R^{L2} is independently selected from the group consisting of: H, halo, CN, and C₁₋₆ alkyl optionally substituted with 1-6 R^{c};
each L^{A3} is independently selected from the group consisting of: —N(R^{d})—, —O—, —S(O)₀₋₂—, and C(═O);
each L^{A4} is independently selected from the group consisting of: C₃₋₁₀ cycloalkylene, 4-10 membered heterocyclylene, C₆₋₁₀ arylene, and 5-10 membered heteroarylene, each of which is optionally substituted with 1-3 R^{g};
Ring B is selected from the group consisting of: C₆₋₁₀ arylene and 5-10 membered heteroarylene, each of which is optionally substituted with 1-6 substituents independently selected from the group consisting of: R^{a} and R^{b};
Y¹ is selected from the group consisting of: a bond, —N(H)—, —N(C₁₋₃ alkyl)-, —N(C₃₋₆ cycloalkyl)-, —O—, and —S(O)₀₋₂—;
n is 0 or 1;
Y² is a straight-chain C₁₋₆ alkylene optionally substituted with 1-6 R^{Y};
each R^{Y} is independently selected from the group consisting of: halo, cyano, —OH, oxo, C₁₋₆ alkoxy, C₁₋₆ haloalkoxy, C₁₋₆ alkyl, and C₁₋₆ haloalkyl, or
a pair of R^{Y} on the same or different carbon atom(s) taken together with the atom(s) connecting them form a C₃₋₆ cycloalkyl ring or 4-6 membered heterocyclyl ring, each of which is optionally substituted with 1-3 independently selected C₁₋₃ alkyl;
R³ is selected from the group consisting of:
(a) —H;
(b) -halo;
(c) —NR^{d}R^{e};
(d) C₃₋₁₀ cycloalkyl or 3-15 membered heterocyclyl, each of which is optionally substituted with 1-6 substituents independently selected from the group consisting of: R^{a} and R^{b}; and
(e) C₆₋₁₀ aryl or 5-10 membered heteroaryl, each of which is optionally substituted with 1-6 substituents independently selected from the group consisting of: R^{a} and R^{b};
E¹ is N, CH, or CR⁶;
R⁴, R⁵, and R⁶ are independently selected from the group consisting of:
(a) —H;
(b) halo;
(c) cyano;
(d) C₁₋₆ alkyl;
(e) C₁₋₆ haloalkyl;
(f) C₁₋₆ alkoxy;
(g) C₁₋₆ haloalkoxy;
(h) —(C₀₋₃ alkylene)-C₃₋₆ cycloalkyl, wherein the C₃₋₆ cycloalkyl portion is optionally substituted with 1-3 R^{g}; and
(i) —O—(C₀₋₃ alkylene)-C₃₋₆ cycloalkyl, wherein the C₃₋₆ cycloalkyl portion is optionally substituted with 1-3 R^{g};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{h})_{b}-R^{b1} and —R^{b1}, wherein:
b is 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, cyano, —OH, —C₁₋₆ alkoxy, —C₁₋₆ haloalkoxy, —NH₂, —N(H)(C₁₋₃ alkyl), and —N(C₁₋₃ alkyl)₂.

In some embodiments, the compounds of Formula (A) are compounds of Formula (I):

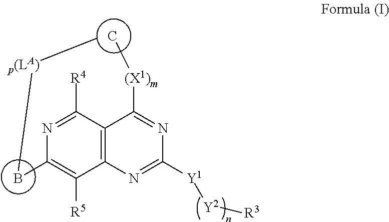

or pharmaceutically acceptable salts thereof, wherein:
m is 0, 1, or 2;
each X¹ is independently selected from the group consisting of CH₂, CHR^{a}, C(R^{a})₂, O, N(R^{d}), C(═O), and S(O)₀₋₂, provided that —(X¹)ₘ— does not include any O—O, O—N, N—S(O)₀, or O—S(O)₀₋₂ bonds;
Ring C is selected from the group consisting of: C₃₋₁₅ cycloalkylene, 4-15 membered heterocyclylene, 6-15 membered arylene, and 5-15 membered heteroarylene, each of which is optionally substituted with 1-4 R⁷, wherein:
each R⁷ is independently selected from the group consisting of R^{a} and R^{b};
p is an integer from 3 to 10;
each L^{A} is independently selected from the group consisting of: L^{A1}, L^{A2}, L^{A3}, and L^{A4}, provided that 0-2 occurrences of L^{A} are L^{A4}, and 0-3 occurrences of L^{A} are selected from the group consisting of L^{A2} and L^{A3};
each L^{A1} is independently selected from the group consisting of: —CH₂—, —CHR^{L}—, and —C(R^{L})₂—, wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, and C₁₋₆ alkyl optionally substituted with 1-6 R^{e};
each L^{A2} is independently selected from the group consisting of:

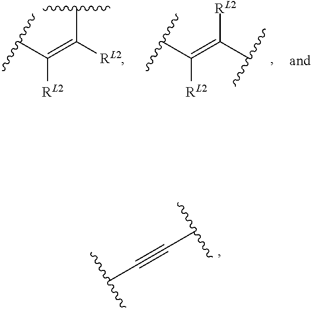

wherein each R^{L2} is independently selected from the group consisting of: H, halo, CN, and C₁₋₆ alkyl optionally substituted with 1-6 R^{c};
each L^{A3} is independently selected from the group consisting of: —N(R^{d})—, —O—, —S(O)₀₋₂—, and C(═O);
each L^{A4} is independently selected from the group consisting of: C₃₋₁₀ cycloalkylene, 4-10 membered heterocyclylene, C₆₋₁₀ arylene, and 5-10 membered heteroarylene, each of which is optionally substituted with 1-3 R^{g};
Ring B is selected from the group consisting of: C₆₋₁₀ arylene and 5-10 membered heteroarylene, each of which is optionally substituted with 1-6 substituents independently selected from the group consisting of: R^{a} and R^{b};
Y¹ is selected from the group consisting of: a bond, —N(H)—, —N(C₁₋₃ alkyl)-, —N(C₃₋₆ cycloalkyl)-, —O—, and —S(O)₀₋₂—;
n is 0 or 1;
Y² is a straight-chain C₁₋₆ alkylene optionally substituted with 1-6 R^{Y};
each R^{Y} is independently selected from the group consisting of: halo, cyano, —OH, oxo, C₁₋₆ alkoxy, C₁₋₆ haloalkoxy, C₁₋₆ alkyl, and C₁₋₆ haloalkyl, or
a pair of R^{Y} on the same or different carbon atom(s) taken together with the atom(s) connecting them form a C₃₋₆ cycloalkyl ring or 4-6 membered heterocyclyl ring, each of which is optionally substituted with 1-3 independently selected C₁₋₃ alkyl;
R³ is selected from the group consisting of:
(a) —H;
(b) -halo;
(c) —NR^{d}R^{e};
(d) C₃₋₁₀ cycloalkyl or 3-15 membered heterocyclyl, each of which is optionally substituted with 1-6 substituents independently selected from the group consisting of: R^{a} and R^{b}; and
(e) C₆₋₁₀ aryl or 5-10 membered heteroaryl, each of which is optionally substituted with 1-6 substituents independently selected from the group consisting of: R^{a} and R^{b};
R⁴ and R⁵ are independently selected from the group consisting of:
(a) —H;
(b) halo;
(c) cyano;
(d) C₁₋₆ alkyl;
(e) C₁₋₆ haloalkyl;
(f) C₁₋₆ alkoxy;
(g) C₁₋₆ haloalkoxy;
(h) —(C₀₋₃ alkylene)-C₃₋₆ cycloalkyl, wherein the C₃₋₆ cycloalkyl portion is optionally substituted with 1-3 R^{g}; and
(i) —O—(C₀₋₃ alkylene)-C₃₋₆ cycloalkyl, wherein the C₃₋₆ cycloalkyl portion is optionally substituted with 1-3 R^{g};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
b is 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, cyano, —OH, —C₁₋₆ alkoxy, —C₁₋₆ haloalkoxy, —NH₂, —N(H)(C₁₋₃ alkyl), and —N(C₁₋₃ alkyl)₂.

In some embodiments, the compounds of Formula (A) are compounds of Formula (II):

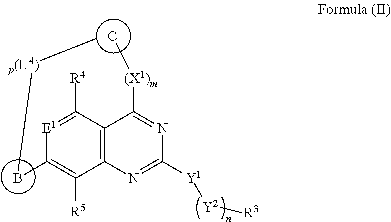

or pharmaceutically acceptable salts thereof, wherein E¹ is CH or CR⁶.

In some embodiments of Formula (A), Formula (I), or Formula (II), m is 0.

In some embodiments of Formula (A), Formula (I), or Formula (II), Ring C is a 4-15 membered heterocyclylene optionally substituted with 1-4 R⁷. In some embodiments, Ring C is a 4-10 membered heterocyclylene optionally substituted with 1-4 R⁷, wherein Ring C has one ring nitrogen atom, 0-1 ring oxygen atom, and no additional ring heteroatoms.

In some embodiments of Formula (A), Formula (I), or Formula (II), Ring C is a 4-6 membered heterocyclylene optionally substituted with 1-3 R⁷. In some embodiments, Ring C is a piperidinylene optionally substituted with 1-3 R⁷.

In some embodiments of Formula (A), Formula (I), or Formula (II), Ring C is:

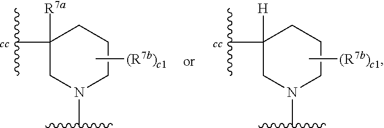

wherein: c1 is 0, 1, or 2; each R^{7a} and R^{7b} is an independently selected R⁷; and cc represents the point of attachment to -(L^{A})ₚ-. In some embodiments, c1 is 0. In some embodiments, Ring C is

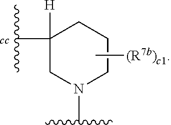

In some embodiments, Ring C is

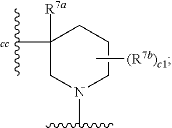

and R^{7a} is C₁₋₃ alkyl optionally substituted with 1-3 F. In some embodiments, Ring C is

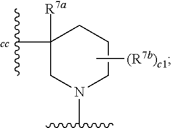

### and R^{7a} is —OH.

In some embodiments of Formula (A), Formula (I), or Formula (II), Ring C is a 7-10 membered heterocyclylene optionally substituted with 1-3 R⁷. In some embodiments, Ring C has one ring nitrogen atom, one ring oxygen atom, and no additional ring heteroatoms.

In some embodiments of Formula (A), Formula (I) or Formula (II), Ring C is

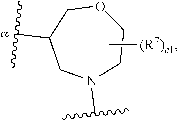

wherein c1 is 0, 1, or 2; and cc represents the point of attachment to -(L^{A})ₚ-. In some embodiments, c1 is 0.

In some embodiments of Formula (A), Formula (I), or Formula (II), p is 4, 5, or 6.

In some embodiments of Formula (A), Formula (I), or Formula (II), p is 6, 7, or 8.

In some embodiments of Formula (A), Formula (I), or Formula (II), each L^{A} is independently selected from the group consisting of: L^{A1}, L^{A3}, and L^{A4}.

In some embodiments of Formula (A), Formula (I), or Formula (II), 0-1 occurrence of L^{A} is L^{A4}; and each remaining L^{A} is independently selected from the group consisting of L^{A1} and L^{A3}.

In some embodiments of Formula (A), Formula (I), or Formula (II), one occurrence of L^{A} is L^{A4}; and each remaining L^{A} is independently L^{A1}.

In some embodiments of Formula (A), Formula (I), or Formula (II), each L^{A} is independently L^{A1}.

In some embodiments of Formula (A), Formula (I), or Formula (II), 1-2 occurrence of L^{A} is independently L^{A3}; and each remaining L^{A} is independently L^{A1}.

In some embodiments of Formula (A), Formula (I), or Formula (II), p is 4, 5, or 6; one occurrence of L^{A} is L^{A4}; and each remaining L^{A} is independently L^{A1}. In some embodiments, -(L^{A})ₚ- is -(L^{A1})₀₋₅-L^{A4}-(L^{A1})₀₋₅, wherein p is 4, 5, or 6. In some embodiments, L^{A4} is phenylene or 5-6 membered heteroarylene, each of which is optionally substituted with 1-3 R^{g}. In some embodiments, L^{A4} is a 5-membered heteroarylene optionally substituted with 1-2 R^{g}. For example, L^{A4} can be triazolylene or oxadiazolylene (e.g., 1,2,4-oxadiazolylene). In some embodiments, L^{A1} is CH₂.

In some embodiments of Formula (A), Formula (I), or Formula (II), -(L^{A})ₚ- is: -(L^{A1a})ₐ₁ₐ-L^{A4}-(L^{A1b})_{a1b-bb}, wherein: a1a and a1b are independently integers from 0 to 5, provided that a1a+a1b is 3, 4, or 5; each L^{A1a} and L^{A1b} is an independently selected L^{A1}; L^{A4} is phenylene or 5-6 membered heteroarylene, each of which is optionally substituted with 1-3 R^{g}; and bb represents the point of attachment to Ring C. In some embodiments, L^{A4} is 5-membered heteroarylene optionally substituted with 1-2 R^{g}. In some embodiments, L^{A4} is selected from the group consisting of:

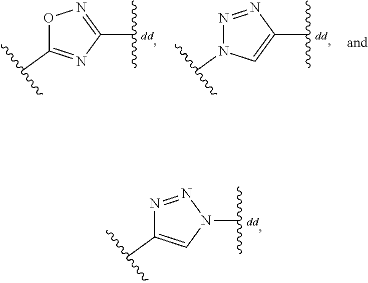

wherein dd represents the point of attachment to -(L^{A1b})_{a1b}-. For example, L^{A4} can be

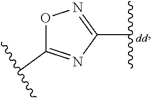

wherein dd represents the point of attachment to -(L^{A1b})_{a1b}-. In some embodiments, a1b is 1; and a1a is 2, 3, or 4. In some embodiments, each of L^{A1a} and L^{A1b} is CH₂. In some embodiments, p is 6, 7, or 8; and each L^{A} is independently L^{A1}. In some embodiments, each L^{A} is —CH₂—.

In some embodiments of Formula (A), Formula (I), or Formula (II), p is 6, 7, or 8; one occurrence of L^{A} is C(═O); a second occurrence of L^{A} is N(R^{d}); and each remaining L^{A} is independently L^{A1}.

In some embodiments of Formula (A), Formula (I), or Formula (II), -(L^{A})ₚ- is: -(L^{A1a})ₐ₁ₐ-C(═O)NH-(L^{A1b})_{a1b-bb}, wherein: a1a and a1b are independently integers from 0 to 5, provided that a1a+a1b is 4, 5, or 6; each L^{A1a} and L^{A1b} is an independently selected L^{A1}; and bb represents the point of attachment to Ring C. In some embodiments, a1b is 2; and a1a is 2, 3, or 4. In some embodiments, a1b is 1; and a1a is 3, 4, or 5. In some embodiments, each of L^{A1a} and L^{A1b} is CH₂.

In some embodiments of Formula (A), Formula (I), or Formula (II), m is 0; Ring C is:

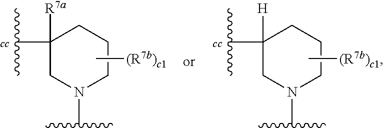

wherein: c1 is 0, 1, or 2; each R^{7a} and R^{7b} is an independently selected R⁷; and cc represents the point of attachment to -(L^{A})ₚ-; -(L^{A})ₚ- is: -(L^{A1a})ₐ₁ₐ-L^{A4}-(L^{A1b})_{a1b-bb}, wherein: a1a and a1b are independently integers from 0 to 5, provided that a1a+a1b is 3, 4, or 5; each L^{A1a} and L^{A1b} is an independently selected L^{A1}; L^{A4} is phenylene or 5-6 membered heteroarylene, each of which is optionally substituted with 1-3 R^{g}; and bb represents the point of attachment to Ring C. In some embodiments, L^{A4} is 5-membered heteroarylene optionally substituted with 1-2 R^{g}. In some embodiments, L^{A4} is

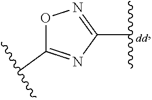

wherein dd represents the point of attachment to -(L^{A1b})_{a1b}-. In some embodiments, a1b is 1; and a1a is 2, 3, or 4. In some embodiments, each of L^{A1a} and L^{A1b} is CH₂. In some embodiments, Ring C is

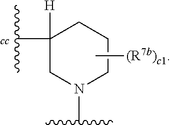

In some embodiments, Ring C is

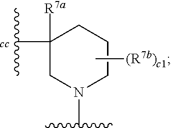

and R^{7a} is —OH. In some embodiments, c1 is 0.

In some embodiments of Formula (A), Formula (I), or Formula (II), m is 0; Ring C is:

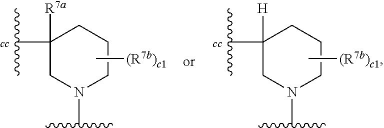

wherein: c1 is 0, 1, or 2; each R^{7a} and R^{7b} is an independently selected R⁷; and cc represents the point of attachment to -(L^{A})ₚ-; p is 6, 7, or 8; and each L^{A} is an independently selected L^{A1}. In some embodiments, each L^{A} is —CH₂—. In some embodiments, Ring C is

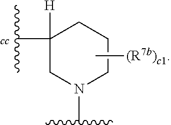

In some embodiments, Ring C is

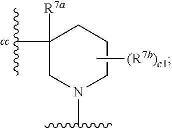

and R^{7a} is —OH. In some embodiments, c1 is 0. In some embodiments, c1 is 0.

In some embodiments of Formula (A), Formula (I), or Formula (II), m is 0; Ring C is:

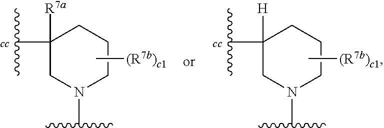

wherein: c1 is 0, 1, or 2; each R^{7a} and R^{7b} is an independently selected R⁷; and cc represents the point of attachment to -(L^{A})ₚ-; -(L^{A})ₚ- is: -(L^{A1a})ₐ₁ₐ-C(═O)NH-(L^{A1b})_{a1b-bb}, wherein: a1a and a1b are independently integers from 0 to 5, provided that a1a+a1b is 4, 5, or 6; each L^{A1a} and L^{A1b} is independently selected L^{A1}; and bb represents the point of attachment to Ring C. In some embodiments, a1b is 2; and a1a is 2, 3, or 4. In some embodiments, a1b is 1; and a1a is 3, 4, or 5. In some embodiments, each of L^{A1a} and L^{A1b} is CH₂. In some embodiments, Ring C is

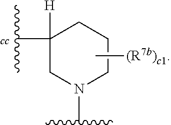

In some embodiments, Ring C is

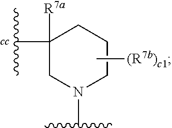

and R^{7a} is —OH. In some embodiments, c1 is 0. In some embodiments, c1 is 0.

In some embodiments of Formula (A), Formula (I), or Formula (II), m is 0; Ring C is:

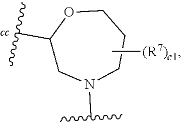

wherein: c1 is 0, 1, or 2; cc represents the point of attachment to -(L^{A})ₚ-; and -(L^{A})ₚ- is: -(L^{A1a})ₐ₁ₐ-C(═O)NH-(L^{A1b})_{a1b-bb}, wherein: a1a and a1b are independently integers from 0 to 5, provided that a1a+a1b is 4, 5, or 6; each L^{A1a} and L^{A1b} is independently selected L^{A1}; and bb represents the point of attachment to Ring C. In some embodiments, a1b is 2; and a1a is 2, 3, or 4. In some embodiments, a1b is 1; and a1a is 3, 4, or 5. In some embodiments, each of L^{A1a} and L^{A1b} is CH₂. In some embodiments, c1 is 0.

In some embodiments of Formula (A), Formula (I), or Formula (II), Ring B is C₆₋₁₀ arylene optionally substituted with 1-6 substituents independently selected from the group consisting of: R^{a} and R^{b}.

In some embodiments of Formula (A), Formula (I), or Formula (II), Ring B is naphthylene optionally substituted with 1-4 R^{a}.

In some embodiments of Formula (A), Formula (I), or Formula (II), Ring B is

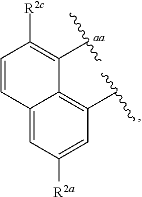

wherein R^{2a} and R^{2c} are independently H or R^{a}; and aa represents the point of attachment to -(L^{A})ₚ-. In some embodiments, R^{2a} is —OH. In some embodiments, R^{2a} is —NH₂. In some embodiments, R^{2a} is —H. In some embodiments, R^{2c} is halo (e.g., F) or H.

In some embodiments of Formula (A), Formula (I), or Formula (II), Ring B is

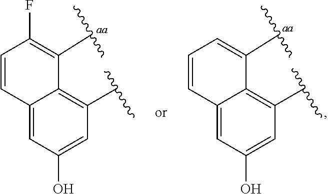

wherein aa represents the point of attachment to -(L^{A})ₚ-.

In some embodiments of Formula (A), Formula (I), or Formula (II), Ring B is

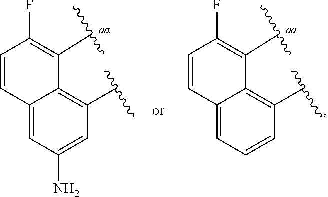

wherein aa represents the point of attachment to -(L^{A})ₚ-.

In some embodiments of Formula (A), Formula (I), or Formula (II), Ring B is phenylene optionally substituted with 1-4 R^{a}.

In some embodiments of Formula (A), Formula (I), or Formula (II), Ring B is

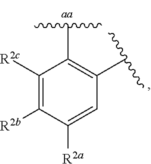

wherein R^{2a}, R^{2b}, and R^{2c} are independently H or R^{a}; and aa represents the point of attachment to -(L^{A})ₚ-. In some embodiments, R^{2a} is —OH. In some embodiments, R^{2a} is —NH₂. In some embodiments, R^{2c} is halo (e.g., —Cl).

In some embodiments of Formula (A), Formula (I), or Formula (II), Ring B is

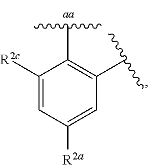

wherein R^{2a} and R^{2c} are independently H or R^{a}; and aa represents the point of attachment to -(L^{A})ₚ-. In some embodiments, R^{2a} is —OH. In some embodiments, R^{2a} is —NH₂. In some embodiments, R^{2c} is halo (e.g., —Cl).

In some embodiments of Formula (A), Formula (I), or Formula (II), Ring B is a 5-10 membered heteroarylene, which is optionally substituted with 1-6 substituents independently selected from the group consisting of: R^{a} and R^{b}. In some embodiments, Ring B is a 9-10 membered heteroarylene having 1-2 ring nitrogen atoms and 0-1 additional ring heteroatom selected from the group consisting of: O and S, wherein the heteroarylene is optionally substituted with 1-3 R^{a}.

In some embodiments of Formula (A), Formula (I), or Formula (II), Ring B is

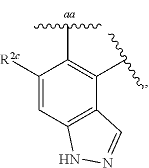

wherein R^{2c} is H or R^{a}; and aa represents the point of attachment to -(L^{A})ₚ-. In some embodiments, R^{2c} is halo. For example, R^{2c} can be —Cl. For example, Ring B can be

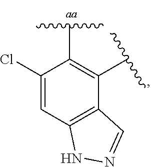

wherein aa represents the point of attachment to -(L^{A})ₚ-.

In some embodiments of Formula (A), Formula (I), or Formula (II), Y¹ is —O—.

In some embodiments of Formula (A), Formula (I), or Formula (II), n is 1.

In some embodiments of Formula (A), Formula (I), or Formula (II), Y² is a straight-chain C₁₋₃ alkylene optionally substituted with 1-3 R^{Y}.

In some embodiments of Formula (A), Formula (I), or Formula (II), Y² is —CH₂—.

In some embodiments of Formula (A), Formula (I), or Formula (II), Y² is

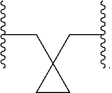 [SMILES: *CC1(C*)CC1]

In some embodiments of Formula (A), Formula (I), or Formula (II), R³ is a 4-10 membered heterocyclyl optionally substituted with 1-6 substituents independently selected from the group consisting of: R^{a} and R^{b}.

In some embodiments of Formula (A), Formula (I), or Formula (II), R³ is a monocyclic 4-6 membered heterocyclyl optionally substituted with 1-3 R^{a}, wherein the heterocyclyl includes at least one ring nitrogen atom.

In some embodiments of Formula (A), Formula (I), or Formula (II), R³ is

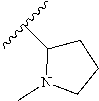 [SMILES: *C1CCCN1C]

optionally substituted with 1-3 R^{a} on one or more ring carbon atoms.

In some embodiments of Formula (A), Formula (I), or Formula (II), R³ is

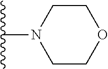 [SMILES: *N1CCOCC1]

optionally substituted with 1-3 R^{a} on one or more ring carbon atoms.

In some embodiments of Formula (A), Formula (I), or Formula (II), R³ is a bicyclic 7-10 membered heterocyclyl optionally substituted with 1-6 R^{a}.

In some embodiments of Formula (A), Formula (I), or Formula (II), R³ is

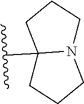 [SMILES: *C12CCCN1CCC2]

optionally substituted with 1-3 R^{a}.

In some embodiments of Formula (A), Formula (I), or Formula (II), R³ is selected from the group consisting of:

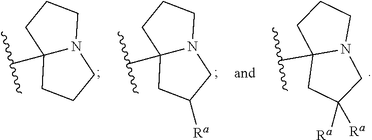

For example, R³ can be

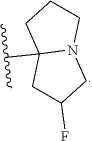 [SMILES: *C12CCCN1CC(F)C2]

In some embodiments of Formula (A), Formula (I), or Formula (II), R³ is selected from the group consisting of:

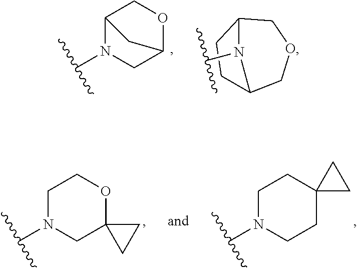

each of which is optionally substituted with 1-3 R^{a}.

In some embodiments of Formula (A), Formula (I), or Formula (II), Y¹ is —O—; n is 1; Y² is —CH₂—; and R³ is

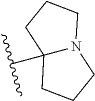 [SMILES: *C12CCCN1CCC2]

optionally substituted with 1-3 R^{a}. In some embodiments, R³ is selected from the group consisting of:

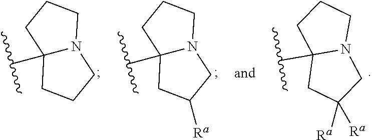

For example, R³ can be

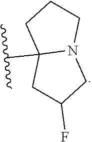 [SMILES: *C12CCCN1CC(F)C2]

In some embodiments of Formula (A), Formula (I), or Formula (II), Y¹ is —O—; n is 1; Y² is

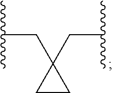 [SMILES: *CC1(C*)CC1]

and R³ is a 5-8 membered heterocyclyl optionally substituted with 1-3 R^{a}. In some embodiments, R³ is selected from the group consisting of:

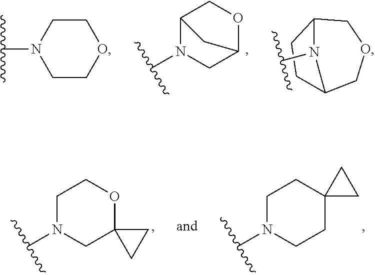

each of which is optionally substituted with 1-3 R^{a}.

In some embodiments of Formula (A), Formula (I), or Formula (II), Y¹ is —O—; n is 1; Y² is

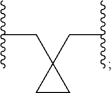 [SMILES: *CC1(C*)CC1]

and R³ is —NR^{d}R^{e}. For example, R³ can be —NMe₂.

In some embodiments of Formula (A), Formula (I), or Formula (II), R⁴ is H.

In some embodiments of Formula (A), Formula (I), or Formula (II), R⁵ is —F.

In some embodiments of Formula (A), Formula (I), or Formula (II), R⁴ is H; and R⁵ is —F.

In some embodiments of Formula (A), Formula (I), or Formula (II), E¹ is CR⁶. In some embodiments, R⁶ is halo (e.g., —F, or —Cl).

In some embodiments of Formula (A), Formula (I), or Formula (II), E¹ is CH.

In some embodiments of Formula (A), the compounds are compounds of Formula (Aa):

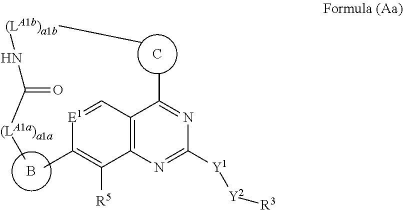

or a pharmaceutically acceptable salt thereof, wherein:
Ring C is a 4-10 membered heterocyclylene optionally substituted with 1-4 R⁷, wherein Ring C has one ring nitrogen atom, 0-1 ring oxygen atom, and no additional ring heteroatoms;
Ring B is selected from the group consisting of: C₆₋₁₀ arylene and 5-10 membered heteroarylene, each of which is optionally substituted with 1-3 substituents independently selected from the group consisting of: R^{a} and R^{b}.
a1a and a1b are independently integers from 0 to 5, provided that a1a+a1b is 4, 5, or 6;
each L^{A1a} and L^{A1b} is an independently selected L^{A1};
R³ is:
a 5-8 membered heterocyclyl optionally substituted with 1-3 R^{a}, or
—NR^{d}R^{e}; and
R⁵ is H or halo.

In some embodiments of Formula (Aa), E¹ is N, CH, or C-halo;
Ring C is selected from the group consisting of:

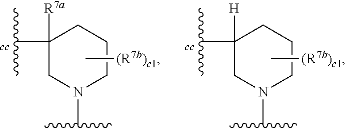

and

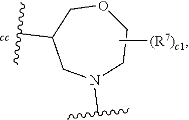

wherein:
c1 is 0, 1, or 2;
cc represents the point of attachment to -(L^{A1b})_{a1b}-;
each R^{7a} and R^{7b} is an independently selected R⁷; and
Ring B is selected from the group consisting of:

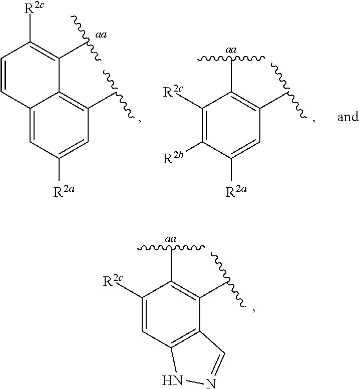

wherein R^{2a} is —OH or —NH₂; R^{2b} and R^{2e} are independently H or halo (e.g., —Cl); and aa represents the point of attachment to -(L^{A1a})ₐ₁ₐ-. In some embodiments, c1 is 0. In some embodiments, R^{7a}, when present, is OH.

In some embodiments of Formula (Aa), a1b is 2; and a1a is 2, 3, or 4. In some embodiments of Formula (Aa), a1b is 1; and a1a is 3, 4, or 5.

In some embodiments of Formula (Aa), each of L^{A1a} and L^{A1b} is CH₂.

In some embodiments of Formula (Aa), Y² is CH₂; R³ is

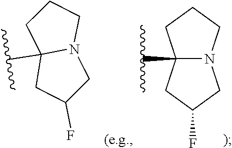 [SMILES: *C12CCCN1CC(F)C2 ; *[C@@]12CCCN1C[C@H](F)C2]

### and R⁵ is F.

In some embodiments of Formula (Aa), Y² is

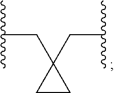 [SMILES: *CC1(C*)CC1]

R³ is —NR^{d}R^{e}; and R⁵ is F. For example, R³ can be —NMe₂.

In some embodiments of Formula (Aa), R⁶ is —F or —Cl; and R⁵ is —F.

In some embodiments of Formula (I), the compounds are compounds of Formula (I-a) or (I-aa):

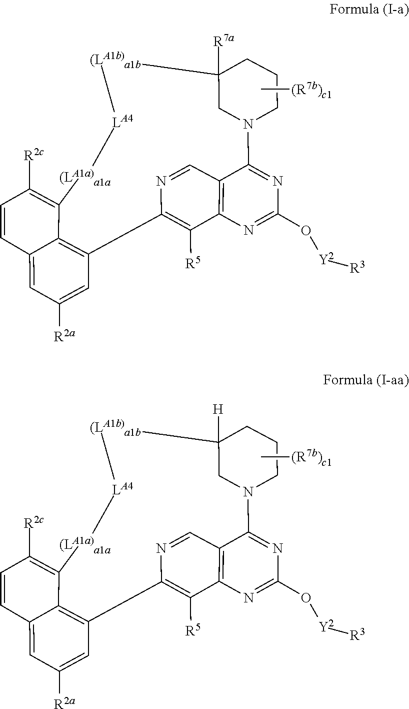

or pharmaceutically acceptable salts thereof, wherein:
c1 is 0, 1, or 2;
each R^{7a} and R^{7b} is an independently selected R⁷;
a1a and a1b are independently integers from 0 to 5, provided that a1a+a1b is 3, 4, or 5;
each L^{A1a} and L^{A1b} is an independently selected L^{A1};
L^{A4} is phenylene or 5-6 membered heteroarylene, each of which is optionally substituted with 1-3 R^{g};
R^{2a} and R^{2c} are independently H or R^{a};
Y² is —CH₂— or

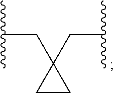 [SMILES: *CC1(C*)CC1]

R³ is a 5-8 membered heterocyclyl optionally substituted with 1-3 R^{a}; and
R⁵ is H or halo.

In some embodiments of Formula (I-a) or (I-aa), L^{A4} is a 5-membered heteroarylene optionally substituted with 1-2 R^{g}. In some embodiments of Formula (I-a) or (I-aa), L^{A4} is

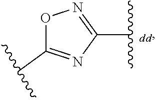

wherein dd represents the point of attachment to -(L^{A1b})_{a1b}-.

In some embodiments of Formula (I-a) or (I-aa), a1b is 1; and a1a is 2, 3, or 4. In some embodiments of Formula (I-a), each of L^{A1a} and L^{A1b} is CH₂.

In some embodiments of Formula (I), the compounds are compounds of Formula (I-b) or (I-bb):

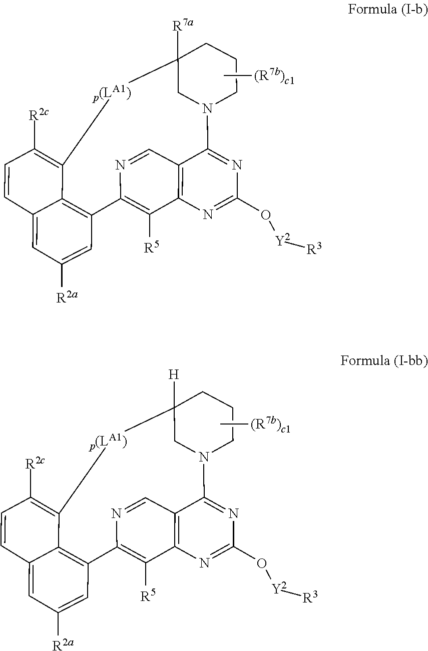

or pharmaceutically acceptable salts thereof, wherein:
c1 is 0, 1, or 2;
each R^{7a} and R^{7b} is an independently selected R⁷;
p is 6, 7, or 8;
R^{2a} and R^{2c} are independently H or R^{a};
Y² is —CH₂— or

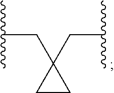 [SMILES: *CC1(C*)CC1]

R³ is a 5-8 membered heterocyclyl optionally substituted with 1-3 R^{a}; and
R⁵ is H or halo.

In some embodiments of Formula (I-b) or (I-bb), each L^{A1} is CH₂.

In some embodiments of Formula (I), the compounds are compounds of Formula (I-c) or (I-cc):

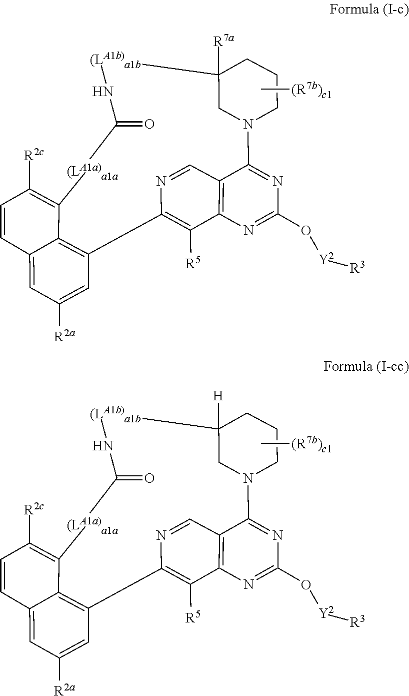

or pharmaceutically acceptable salts thereof, wherein:
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2a} and R^{2c} are independently H or R^{a};
Y² is —CH₂— or

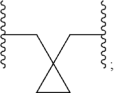 [SMILES: *CC1(C*)CC1]

R³ is a 5-8 membered heterocyclyl optionally substituted with 1-3 R^{a}; and
R⁵ is H or halo.

In some embodiments of Formula (I-c) or (I-cc), a1b is 2; and a1a is 2, 3, or 4.

In some embodiments of Formula (I-c) or (I-cc), each of L^{A1a} and L^{A1b} is CH₂.

In some embodiments of Formula (II), the compounds are compounds of Formula (II-c) or (II-cc):

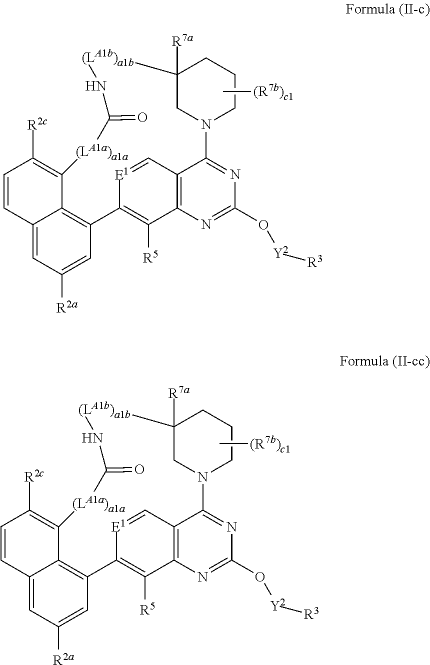

or pharmaceutically acceptable salts thereof, wherein:
E¹ is CH or CR⁶;
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2a} and R^{2c} are independently H or R^{a};
Y² is —CH₂— or

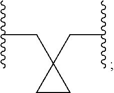 [SMILES: *CC1(C*)CC1]

R³ is a 5-8 membered heterocyclyl optionally substituted with 1-3 R^{a}; and
R⁵ is H or halo.

In some embodiments of Formula (I), the compounds are compounds of Formula (I-d) or (I-dd):

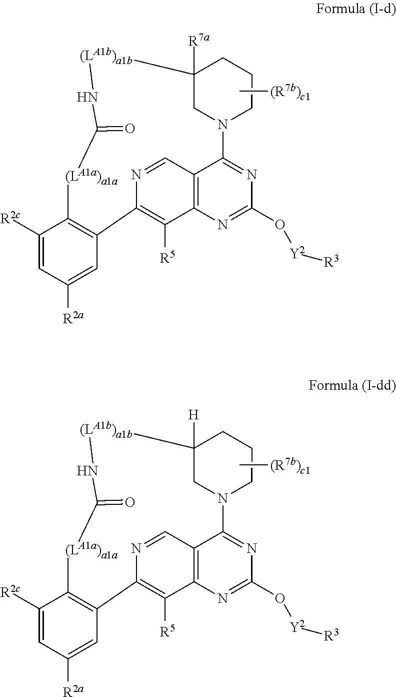

or pharmaceutically acceptable salts thereof, wherein:
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2a} and R^{2c} are independently R^{a};
Y² is —CH₂— or

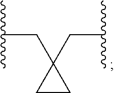 [SMILES: *CC1(C*)CC1]

R³ is a 5-8 membered heterocyclyl optionally substituted with 1-3 R^{a}; and
R⁵ is H or halo.

In some embodiments of Formula (II), the compounds are compounds of Formula (II-d) or (II-dd):

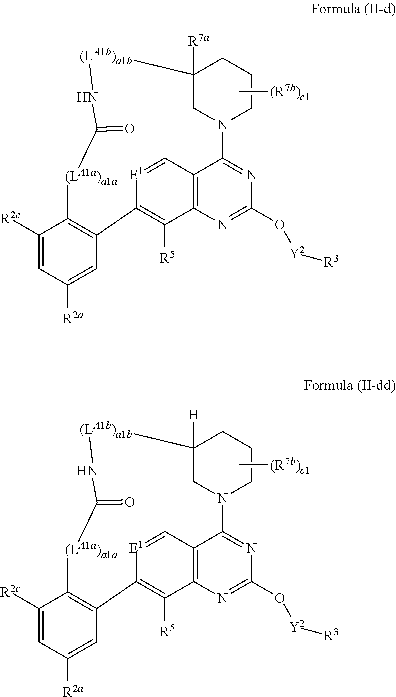

or pharmaceutically acceptable salts thereof, wherein:
E¹ is CH or CR⁶;
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2a} and R^{2c} are independently R^{a};
Y² is —CH₂— or

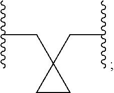 [SMILES: *CC1(C*)CC1]

R³ is a 5-8 membered heterocyclyl optionally substituted with 1-3 R^{a}; and
R⁵ is H or halo.

In some embodiments of Formula (I), the compounds are compounds of Formula (I-e) or (I-ee):

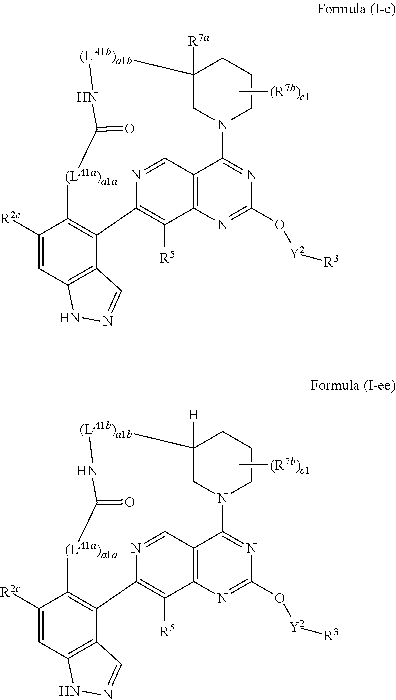

or pharmaceutically acceptable salts thereof, wherein:
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2c} is H or R^{a};
Y² is —CH₂— or

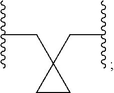 [SMILES: *CC1(C*)CC1]

R³ is:
a 5-8 membered heterocyclyl optionally substituted with 1-3 R^{a}, or
—NR^{d}R^{e} (e.g., —NMe₂); and
R⁵ is H or halo.

In some embodiments of Formula (II), the compounds are compounds of Formula (II-e) or (II-ee):

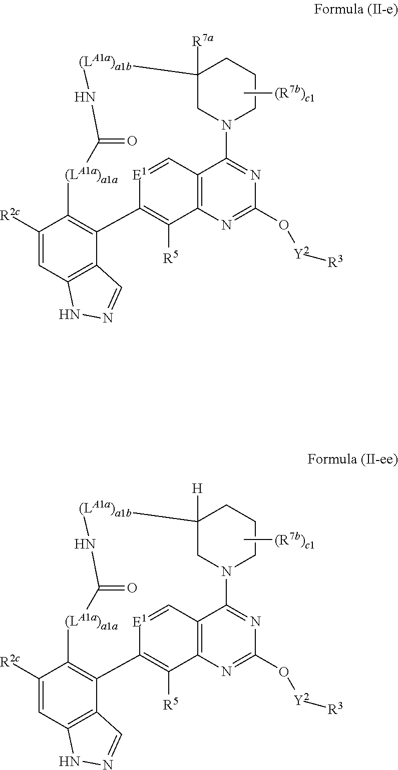

or pharmaceutically acceptable salts thereof, wherein:
E¹ is CH or CR⁶;
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2c} is H or R^{a};
Y² is —CH₂— or

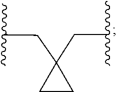 [SMILES: *CC1(C*)CC1]

R³ is:
a 5-8 membered heterocyclyl optionally substituted with 1-3 R^{a}, or
—NR^{d}R^{e} (e.g., —NMe₂); and
R⁵ is H or halo.

In some embodiments of Formula (I), the compounds are compounds of Formula (I-f):

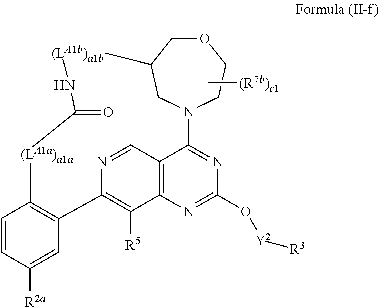

or pharmaceutically acceptable salts thereof, wherein:
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2a} and R^{2c} are independently R^{a};
Y² is —CH₂— or

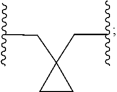 [SMILES: *CC1(C*)CC1]

R³ is a 5-8 membered heterocyclyl optionally substituted with 1-3 R^{a}; and
R⁵ is H or halo.

In some embodiments of Formula (II), the compounds are compounds of Formula (II-f):

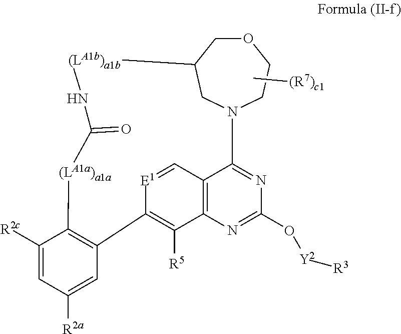

or pharmaceutically acceptable salts thereof, wherein:
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2a} and R^{2c} are independently R^{a};
Y² is —CH₂— or

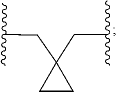 [SMILES: *CC1(C*)CC1]

R³ is a 5-8 membered heterocyclyl optionally substituted with 1-3 R^{a}; and
R⁵ is H or halo.

In some embodiments of Formula (I), the compounds are compounds of Formula (I-g):

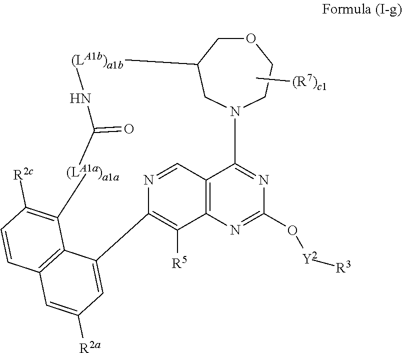

or pharmaceutically acceptable salts thereof, wherein:
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2a} and R^{2c} are independently H or R^{a};
Y² is —CH₂— or

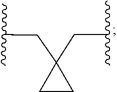 [SMILES: *CC1(C*)CC1]

R³ is a 5-8 membered heterocyclyl optionally substituted with 1-3 R^{a}; and
R⁵ is H or halo.

In some embodiments of Formula (II), the compounds are compounds of Formula (II-g):

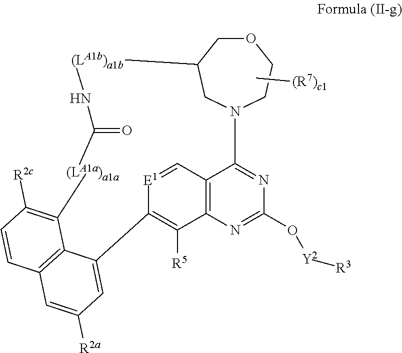

or pharmaceutically acceptable salts thereof, wherein:
E¹ is CH or CR⁶;
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2a} and R^{2c} are independently H or R^{a};
Y² is —CH₂— or

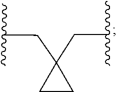 [SMILES: *CC1(C*)CC1]

R³ is a 5-8 membered heterocyclyl optionally substituted with 1-3 R^{a}; and
R⁵ is H or halo.

In some embodiments of Formula (I), the compounds are compounds of Formula (I-h):

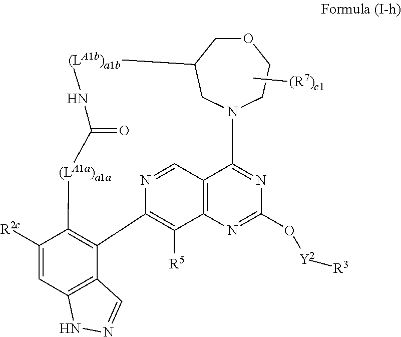

or pharmaceutically acceptable salts thereof, wherein:
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2c} is H or R^{a};
Y² is —CH₂— or

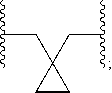 [SMILES: *CC1(C*)CC1]

R³ is:
a 5-8 membered heterocyclyl optionally substituted with 1-3 R^{a}, or
—NR^{d}R^{e} (e.g., —NMe₂); and
R⁵ is H or halo.

In some embodiments of Formula (II), the compounds are compounds of Formula (II-h):

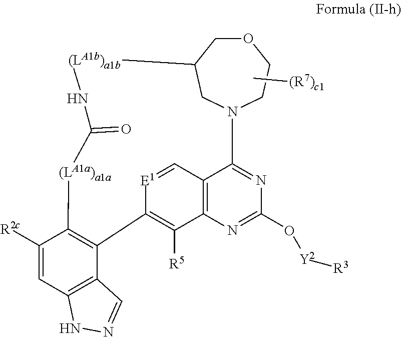

or pharmaceutically acceptable salts thereof, wherein:
E¹ is CH or CR⁶;
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2c} is H or R^{a};
Y² is —CH₂— or

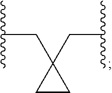 [SMILES: *CC1(C*)CC1]

R³ is:
a 5-8 membered heterocyclyl optionally substituted with 1-3 R^{a}, or
—NR^{d}R^{e} (e.g., —NMe₂); and
R⁵ is H or halo.

In some embodiments of Formula (I-a), (I-aa), (I-b), (I-bb), (I-c), (I-cc), (II-c), (II-cc), (I-d), (I-dd), (II-d), (II-dd), (I-e), (I-ee), (II-e), (II-ee), (I-f), (II-f), (I-g), (II-g), (I-h), or (II-h), a1b is 2; and a1a is 2, 3, or 4.

In some embodiments of Formula (I-a), (I-aa), (I-b), (I-bb), (I-c), (I-cc), (II-c), (II-cc), (I-d), (I-dd), (II-d), (II-dd), (I-e), (I-ee), (II-e), (II-ee), (I-f), (II-f), (I-g), (II-g), (I-h), or (I-h), a1b is 1; and a1a is 3, 4, or 5.

In some embodiments of Formula (I-a), (I-aa), (I-b), (I-bb), (I-c), (I-cc), (II-c), (II-cc), (I-d), (I-dd), (II-d), (II-dd), (I-e), (I-ee), (II-e), (II-ee), (I-f), (II-f), (I-g), (II-g), (I-h), or (II-h), each of L^{A1a} and L^{A1b} is CH₂.

In some embodiments of Formula (I-a), (I-aa), (I-b), (I-bb), (I-c), (I-cc), (II-c), (II-cc), (I-d), (I-dd), (II-d), (II-dd), (I-f), (II-f), (I-g), or (II-g), R^{2a} is —NH₂.

In some embodiments of Formula (I-a), (I-aa), (I-b), (I-bb), (I-c), (I-cc), (II-c), (II-cc), (I-d), (I-dd), (II-d), (II-dd), (I-e), (I-ee), (II-e), (II-ee), (I-f), (II-f), (I-g), (II-g), (I-h), or (I-h), R^{2c} is —F. In some embodiments of Formula (I-a), (I-aa), (I-b), (I-bb), (I-c), (I-cc), (II-c), (II-cc), (I-d), (I-dd), (II-d), (II-dd), (I-e), (I-ee), (II-e), (II-ee), (I-f), (II-f), (I-g), (II-g), (I-h), or (I-h), R^{2c} is —Cl.

In some embodiments of Formula (I-a), (I-aa), (I-b), (I-bb), (I-c), (I-cc), (II-c), (II-cc), (I-d), (I-dd), (II-d), (II-dd), (I-e), (I-ee), (II-e), (II-ee), (I-f), (II-f), (I-g), (II-g), (I-h), or (II-h), c1 is 0.

In some embodiments of Formula (I-a), (I-aa), (I-b), (I-bb), (I-c), (I-cc), (II-c), (II-cc), (I-d), (I-dd), (II-d), (II-dd), (I-e), (I-ee), (II-e), or (II-ee), R^{7a}, when present, is OH.

In some embodiments of Formula (I-a), (I-aa), (I-b), (I-bb), (I-c), (I-cc), (II-c), (II-cc), (I-d), (I-dd), (II-d), (II-dd), (I-e), (I-ee), (II-e), or (II-ee), Y² is CH₂; R³ is

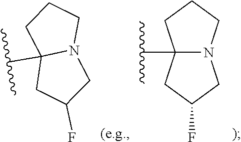 [SMILES: *C12CCCN1CC(F)C2 ; *C12CCCN1C[C@H](F)C2]

### and R⁵ is F.

In some embodiments of Formula (I-e), (I-ee), (II-e), (II-ee), (I-h), or (II-h), Y² is

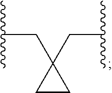 [SMILES: *CC1(C*)CC1]

R³ is —NR^{d}R^{e}; and R⁵ is F. For example, R³ can be —NMe₂.

In some embodiments of Formula (I-a), (I-aa), (I-b), (I-bb), (I-c), (I-cc), (II-c), (II-cc), (I-d), (I-dd), (II-d), (II-dd), (I-e), (I-ee), (II-e), or (II-ee), R⁶ is —F or —Cl; and R⁵ is —F.

In some embodiments, the compounds are selected from the group consisting of the compounds in Table C1, or pharmaceutically acceptable salts thereof.

TABLE C1

| Compound | |
|---|---|
| No. | Structure |
| 101 | 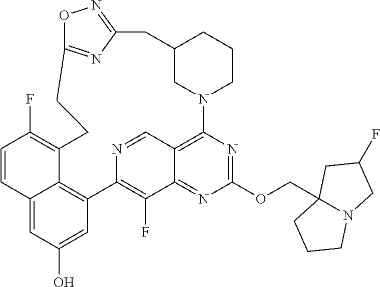 [SMILES: Oc1cc2c3c(c(F)ccc3c1)CCc1nc(no1)CC1CCCN(C1)c1nc(OCC34CCCN3CC(F)C4)nc3c(F)c-2ncc13] |
| 101a | 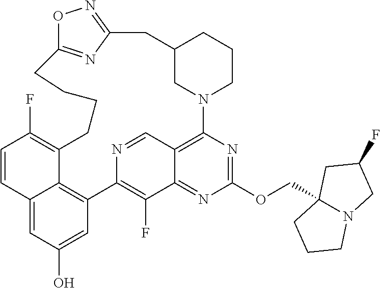 [SMILES: Oc1cc2c3c(c(F)ccc3c1)CCCCc1nc(no1)CC1CCCN(C1)c1nc(OC[C@@]34CCCN3C[C@H](F)C4)nc3c(F)c-2ncc13] |
| 102 | 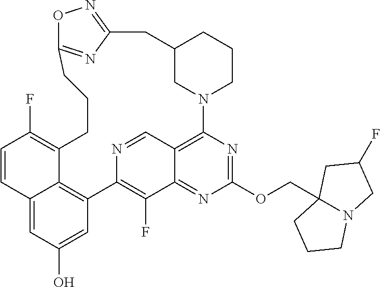 [SMILES: Oc1cc2c3c(c(F)ccc3c1)CCCc1nc(no1)CC1CCCN(C1)c1nc(OCC34CCCN3CC(F)C4)nc3c(F)c-2ncc13] |
| 102a | 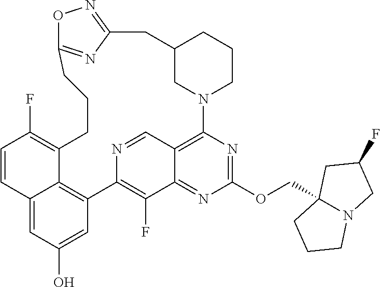 [SMILES: Oc1cc2c3c(c(F)ccc3c1)CCCc1nc(no1)CC1CCCN(C1)c1nc(OC[C@@]34CCCN3C[C@H](F)C4)nc3c(F)c-2ncc13] |
| 103 | 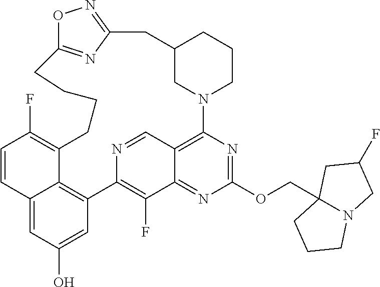 [SMILES: Oc1cc2c3c(c(F)ccc3c1)CCCCc1nc(no1)CC1CCCN(C1)c1nc(OCC34CCCN3CC(F)C4)nc3c(F)c-2ncc13] |
| 103a | 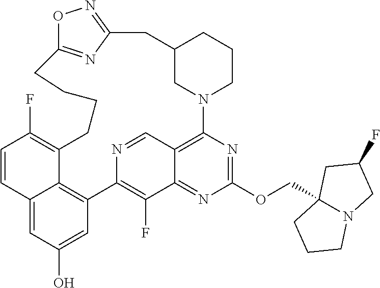 [SMILES: Oc1cc2c3c(c(F)ccc3c1)CCCCc1nc(no1)CC1CCCN(C1)c1nc(OC[C@@]34CCCN3C[C@H](F)C4)nc3c(F)c-2ncc13] |
| 104 | 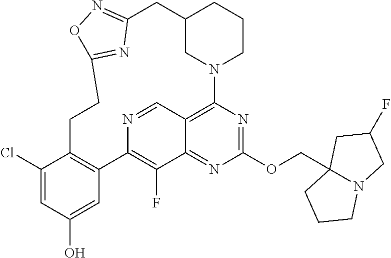 [SMILES: Oc1cc(Cl)c2c(c1)-c1ncc3c(nc(OCC45CCCN4CC(F)C5)nc3c1F)N1CCCC(Cc3noc(n3)CC2)C1] |
| 104a | 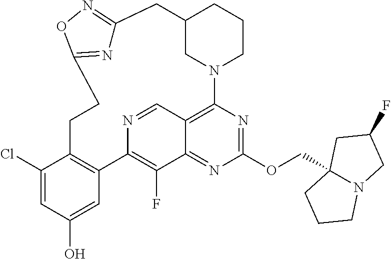 [SMILES: Oc1cc(Cl)c2c(c1)-c1ncc3c(nc(OC[C@@]45CCCN4C[C@H](F)C5)nc3c1F)N1CCCC(Cc3noc(n3)CC2)C1] |
| 105 | 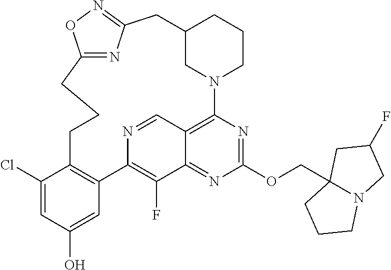 [SMILES: Oc1cc(Cl)c2c(c1)-c1ncc3c(nc(OCC45CCCN4CC(F)C5)nc3c1F)N1CCCC(Cc3noc(n3)CCC2)C1] |
| 105a | 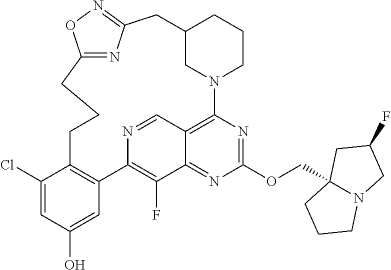 [SMILES: Oc1cc(Cl)c2c(c1)-c1ncc3c(nc(OC[C@@]45CCCN4C[C@H](F)C5)nc3c1F)N1CCCC(Cc3noc(n3)CCC2)C1] |
| 106 | 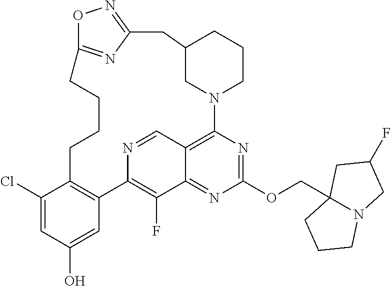 [SMILES: Oc1cc(Cl)c2c(c1)-c1ncc3c(nc(OCC45CCCN4CC(F)C5)nc3c1F)N1CCCC(Cc3noc(n3)CCCC2)C1] |
| 106a | 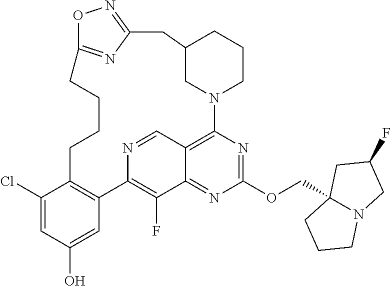 [SMILES: Oc1cc(Cl)c2c(c1)-c1ncc3c(nc(OC[C@@]45CCCN4C[C@H](F)C5)nc3c1F)N1CCCC(Cc3noc(n3)CCCC2)C1] |
| 107 | 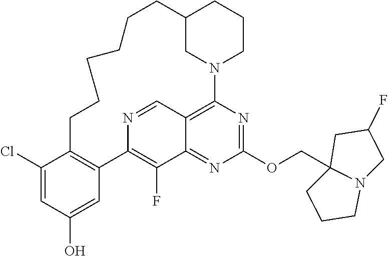 [SMILES: Oc1cc(Cl)c2c(c1)-c1ncc3c(nc(OCC45CCCN4CC(F)C5)nc3c1F)N1CCCC(CCCCCC2)C1] |
| 107a | 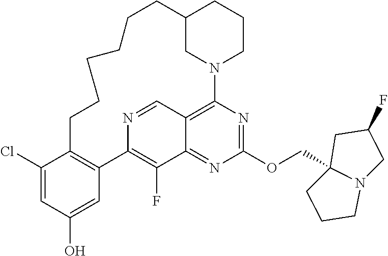 [SMILES: Oc1cc(Cl)c2c(c1)-c1ncc3c(nc(OC[C@@]45CCCN4C[C@H](F)C5)nc3c1F)N1CCCC(CCCCCC2)C1] |
| 108 | 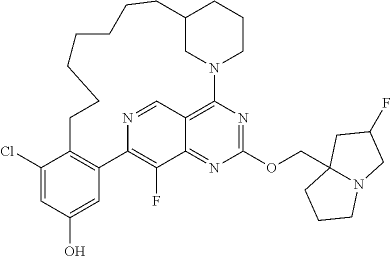 [SMILES: Oc1cc(Cl)c2c(c1)-c1ncc3c(nc(OCC45CCCN4CC(F)C5)nc3c1F)N1CCCC(CCCCCCC2)C1] |
| 108a | 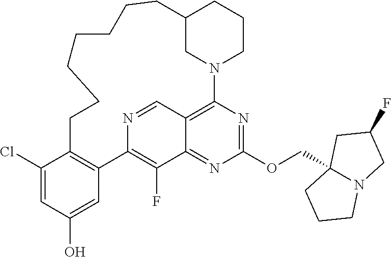 [SMILES: Oc1cc(Cl)c2c(c1)-c1ncc3c(nc(OC[C@@]45CCCN4C[C@H](F)C5)nc3c1F)N1CCCC(CCCCCCC2)C1] |
| 109 | 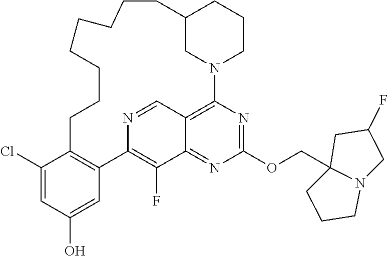 [SMILES: Oc1cc(Cl)c2c(c1)-c1ncc3c(nc(OCC45CCCN4CC(F)C5)nc3c1F)N1CCCC(CCCCCCCC2)C1] |
| 109a | 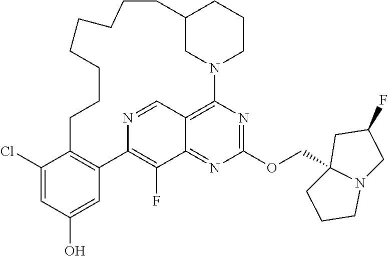 [SMILES: Oc1cc(Cl)c2c(c1)-c1ncc3c(nc(OC[C@@]45CCCN4C[C@H](F)C5)nc3c1F)N1CCCC(CCCCCCCC2)C1] |
| 110 | 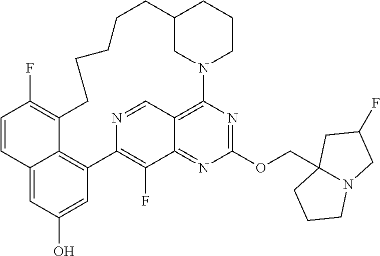 [SMILES: Oc1cc2c3c(c(F)ccc3c1)CCCCCC1CCCN(C1)c1nc(OCC34CCCN3CC(F)C4)nc3c(F)c-2ncc13] |
| 110a | 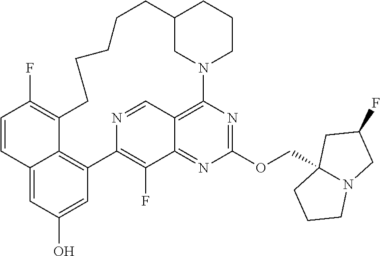 [SMILES: Oc1cc2c3c(c(F)ccc3c1)CCCCCC1CCCN(C1)c1nc(OC[C@@]34CCCN3C[C@H](F)C4)nc3c(F)c-2ncc13] |
| 111 | 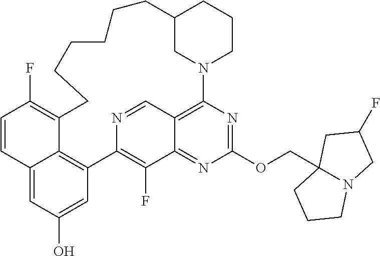 [SMILES: Oc1cc2c3c(c(F)ccc3c1)CCCCCCC1CCCN(C1)c1nc(OCC34CCCN3CC(F)C4)nc3c(F)c-2ncc13] |
| 111a | 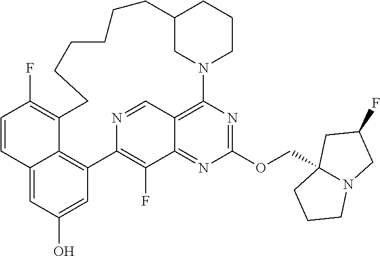 [SMILES: Oc1cc2c3c(c(F)ccc3c1)CCCCCCC1CCCN(C1)c1nc(OC[C@@]34CCCN3C[C@H](F)C4)nc3c(F)c-2ncc13] |
| 112 | 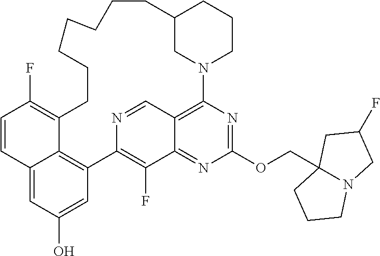 [SMILES: Oc1cc2c3c(c(F)ccc3c1)CCCCCCCC1CCCN(C1)c1nc(OCC34CCCN3CC(F)C4)nc3c(F)c-2ncc13] |
| 112a | 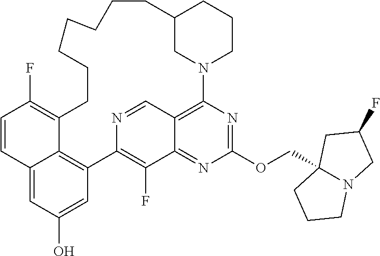 [SMILES: Oc1cc2c3c(c(F)ccc3c1)CCCCCCCC1CCCN(C1)c1nc(OC[C@@]34CCCN3C[C@H](F)C4)nc3c(F)c-2ncc13] |
| 113 | 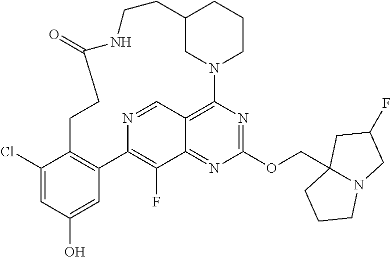 [SMILES: O=C1CCc2c(Cl)cc(O)cc2-c2ncc3c(nc(OCC45CCCN4CC(F)C5)nc3c2F)N2CCCC(CCN1)C2] |
| 113a | 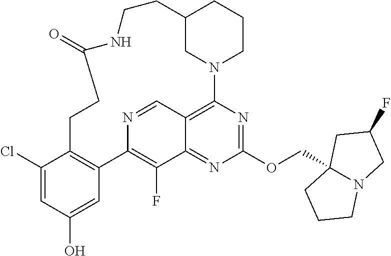 [SMILES: O=C1CCc2c(Cl)cc(O)cc2-c2ncc3c(nc(OC[C@@]45CCCN4C[C@H](F)C5)nc3c2F)N2CCCC(CCN1)C2] |
| 114 | 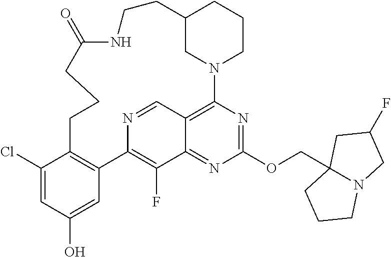 [SMILES: O=C1CCCc2c(Cl)cc(O)cc2-c2ncc3c(nc(OCC45CCCN4CC(F)C5)nc3c2F)N2CCCC(CCN1)C2] |
| 114a | 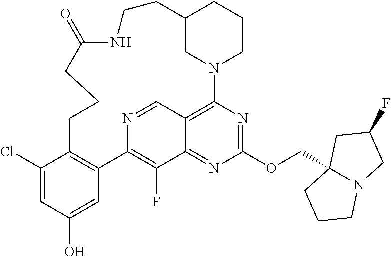 [SMILES: O=C1CCCc2c(Cl)cc(O)cc2-c2ncc3c(nc(OC[C@@]45CCCN4C[C@H](F)C5)nc3c2F)N2CCCC(CCN1)C2] |
| 115 | 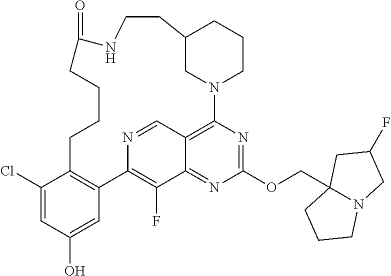 [SMILES: O=C1CCCCc2c(Cl)cc(O)cc2-c2ncc3c(nc(OCC45CCCN4CC(F)C5)nc3c2F)N2CCCC(CCN1)C2] |
| 115a | 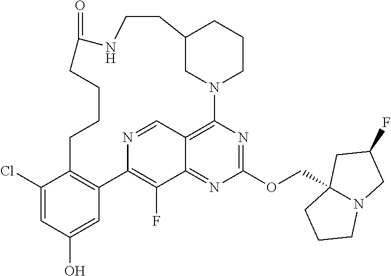 [SMILES: O=C1CCCCc2c(Cl)cc(O)cc2-c2ncc3c(nc(OC[C@@]45CCCN4C[C@H](F)C5)nc3c2F)N2CCCC(CCN1)C2] |
| 115b | 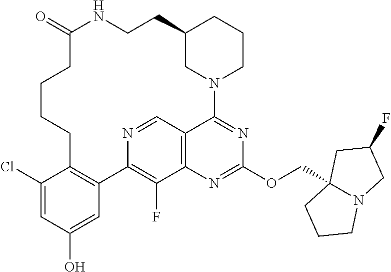 [SMILES: O=C1CCCCc2c(Cl)cc(O)cc2-c2ncc3c(nc(OC[C@@]45CCCN4C[C@H](F)C5)nc3c2F)N2CCC[C@H](CCN1)C2] |
| 115c | 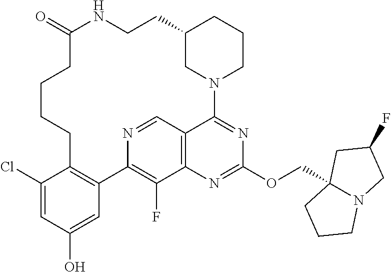 [SMILES: O=C1CCCCc2c(Cl)cc(O)cc2-c2ncc3c(nc(OC[C@@]45CCCN4C[C@H](F)C5)nc3c2F)N2CCC[C@@H](CCN1)C2] |
| 116 | 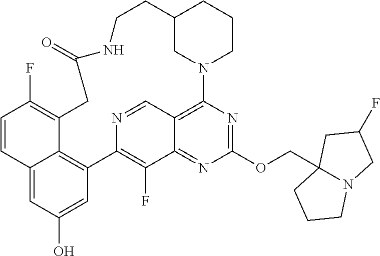 [SMILES: O=C1Cc2c(F)ccc3cc(O)cc(c23)-c2ncc3c(nc(OCC45CCCN4CC(F)C5)nc3c2F)N2CCCC(CCN1)C2] |
| 116a | 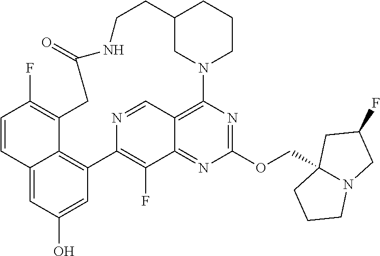 [SMILES: O=C1Cc2c(F)ccc3cc(O)cc(c23)-c2ncc3c(nc(OC[C@@]45CCCN4C[C@H](F)C5)nc3c2F)N2CCCC(CCN1)C2] |
| 117 | 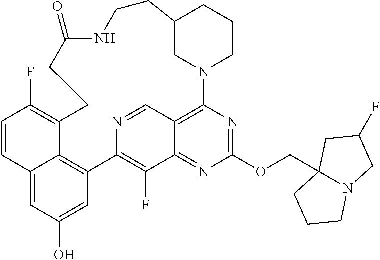 [SMILES: O=C1CCc2c(F)ccc3cc(O)cc(c23)-c2ncc3c(nc(OCC45CCCN4CC(F)C5)nc3c2F)N2CCCC(CCN1)C2] |
| 117a | 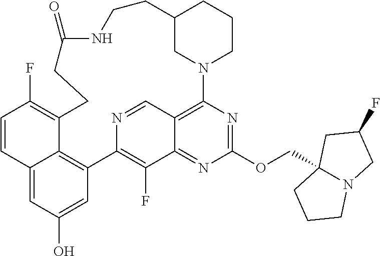 [SMILES: O=C1CCc2c(F)ccc3cc(O)cc(c23)-c2ncc3c(nc(OC[C@@]45CCCN4C[C@H](F)C5)nc3c2F)N2CCCC(CCN1)C2] |
| 118 | 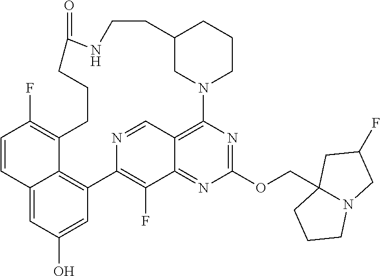 [SMILES: O=C1CCCc2c(F)ccc3cc(O)cc(c23)-c2ncc3c(nc(OCC45CCCN4CC(F)C5)nc3c2F)N2CCCC(CCN1)C2] |
| 118a | 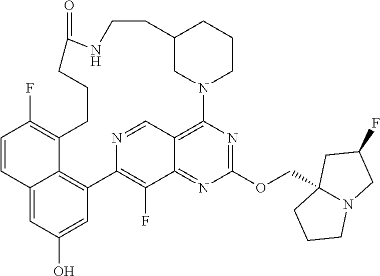 [SMILES: O=C1CCCc2c(F)ccc3cc(O)cc(c23)-c2ncc3c(nc(OC[C@@]45CCCN4C[C@H](F)C5)nc3c2F)N2CCCC(CCN1)C2] |
| 119 | 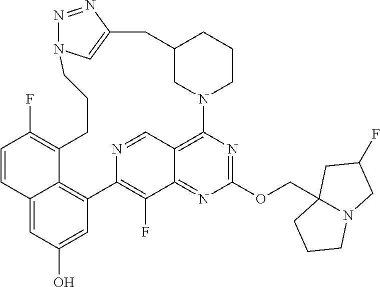 [SMILES: Oc1cc2c3c(c(F)ccc3c1)CCCn1cc(nn1)CC1CCCN(C1)c1nc(OCC34CCCN3CC(F)C4)nc3c(F)c-2ncc13] |
| 119a | 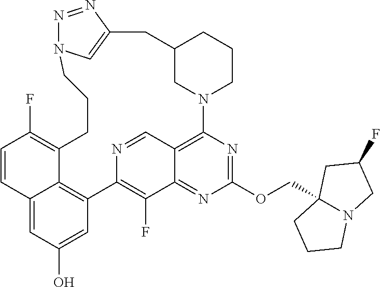 [SMILES: Oc1cc2c3c(c(F)ccc3c1)CCCn1cc(nn1)CC1CCCN(C1)c1nc(OC[C@@]34CCCN3C[C@H](F)C4)nc3c(F)c-2ncc13] |
| 120 | 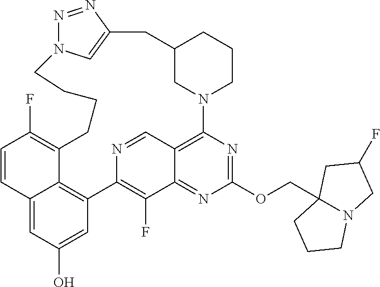 [SMILES: Oc1cc2c3c(c(F)ccc3c1)CCCCn1cc(nn1)CC1CCCN(C1)c1nc(OCC34CCCN3CC(F)C4)nc3c(F)c-2ncc13] |
| 120a | 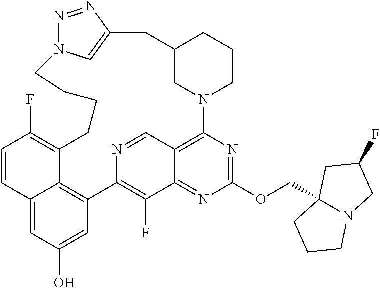 [SMILES: Oc1cc2c3c(c(F)ccc3c1)CCCCn1cc(nn1)CC1CCCN(C1)c1nc(OC[C@@]34CCCN3C[C@H](F)C4)nc3c(F)c-2ncc13] |
| 121 | 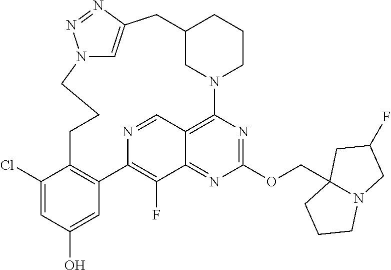 [SMILES: Oc1cc(Cl)c2c(c1)-c1ncc3c(nc(OCC45CCCN4CC(F)C5)nc3c1F)N1CCCC(Cc3cn(nn3)CCC2)C1] |
| 121a | 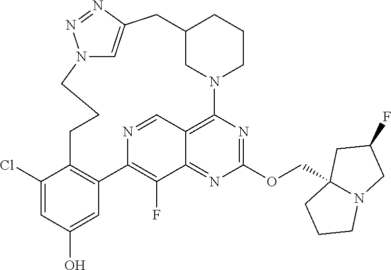 [SMILES: Oc1cc(Cl)c2c(c1)-c1ncc3c(nc(OC[C@@]45CCCN4C[C@H](F)C5)nc3c1F)N1CCCC(Cc3cn(nn3)CCC2)C1] |
| 122 | 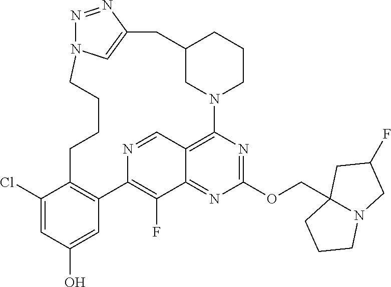 [SMILES: Oc1cc(Cl)c2c(c1)-c1ncc3c(nc(OCC45CCCN4CC(F)C5)nc3c1F)N1CCCC(Cc3cn(nn3)CCCC2)C1] |
| 122a | 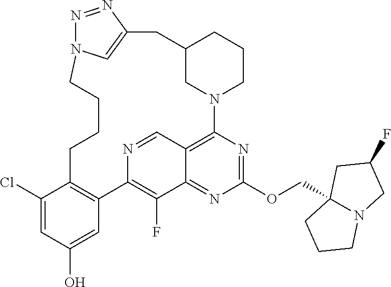 [SMILES: Oc1cc(Cl)c2c(c1)-c1ncc3c(nc(OC[C@@]45CCCN4C[C@H](F)C5)nc3c1F)N1CCCC(Cc3cn(nn3)CCCC2)C1] |
| 123 | 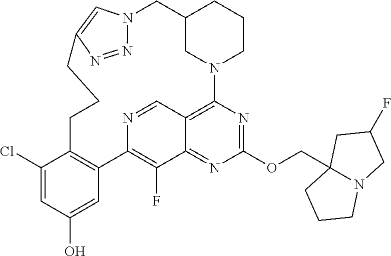 [SMILES: Oc1cc(Cl)c2c(c1)-c1ncc3c(nc(OCC45CCCN4CC(F)C5)nc3c1F)N1CCCC(C1)Cn1cc(nn1)CCC2] |
| 123a | 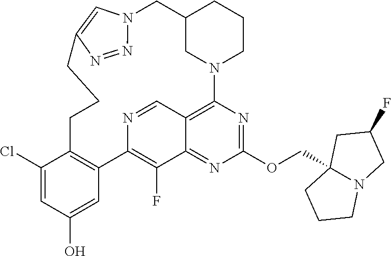 [SMILES: Oc1cc(Cl)c2c(c1)-c1ncc3c(nc(OC[C@@]45CCCN4C[C@H](F)C5)nc3c1F)N1CCCC(C1)Cn1cc(nn1)CCC2] |
| 124 | 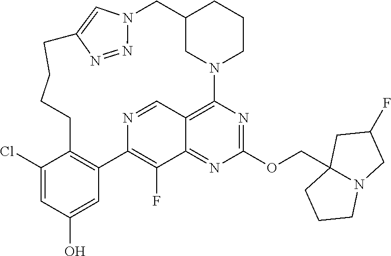 [SMILES: Oc1cc(Cl)c2c(c1)-c1ncc3c(nc(OCC45CCCN4CC(F)C5)nc3c1F)N1CCCC(C1)Cn1cc(nn1)CCCC2] |
| 124a | 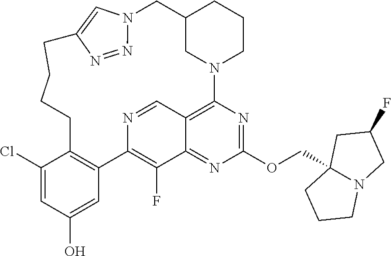 [SMILES: Oc1cc(Cl)c2c(c1)-c1ncc3c(nc(OC[C@@]45CCCN4C[C@H](F)C5)nc3c1F)N1CCCC(C1)Cn1cc(nn1)CCCC2] |
| 125 | 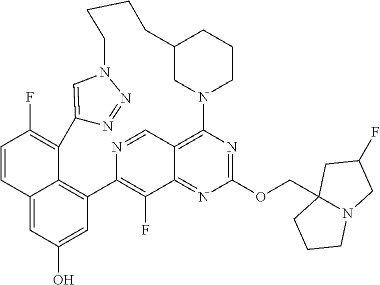 [SMILES: Oc1cc2c3c(c(F)ccc3c1)-c1cn(nn1)CCCCC1CCCN(C1)c1nc(OCC34CCCN3CC(F)C4)nc3c(F)c-2ncc13] |
| 125a | 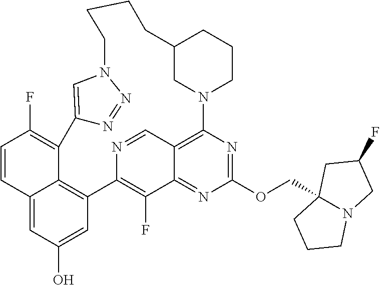 [SMILES: Oc1cc2c3c(c(F)ccc3c1)-c1cn(nn1)CCCCC1CCCN(C1)c1nc(OC[C@@]34CCCN3C[C@H](F)C4)nc3c(F)c-2ncc13] |
| 126 | 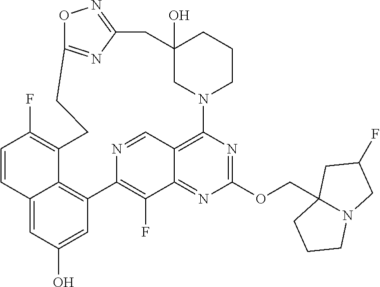 [SMILES: Oc1cc2c3c(c(F)ccc3c1)CCc1nc(no1)CC1(O)CCCN(C1)c1nc(OCC34CCCN3CC(F)C4)nc3c(F)c-2ncc13] |
| 126a | 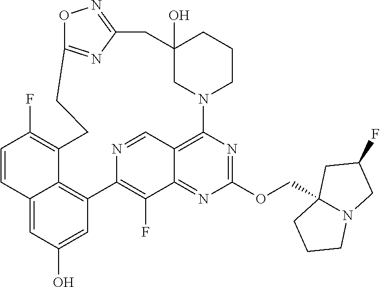 [SMILES: Oc1cc2c3c(c(F)ccc3c1)CCc1nc(no1)CC1(O)CCCN(C1)c1nc(OC[C@@]34CCCN3C[C@H](F)C4)nc3c(F)c-2ncc13] |
| 127 | 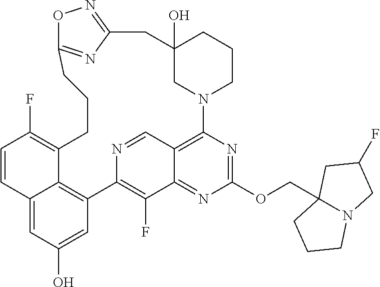 [SMILES: Oc1cc2c3c(c(F)ccc3c1)CCCc1nc(no1)CC1(O)CCCN(C1)c1nc(OCC34CCCN3CC(F)C4)nc3c(F)c-2ncc13] |
| 127a | 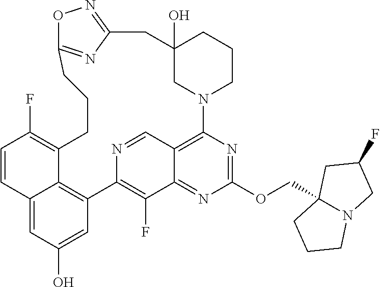 [SMILES: Oc1cc2c3c(c(F)ccc3c1)CCCc1nc(no1)CC1(O)CCCN(C1)c1nc(OC[C@@]34CCCN3C[C@H](F)C4)nc3c(F)c-2ncc13] |
| 128 | 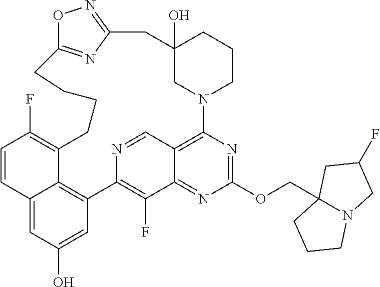 [SMILES: Oc1cc2c3c(c(F)ccc3c1)CCCCc1nc(no1)CC1(O)CCCN(C1)c1nc(OCC34CCCN3CC(F)C4)nc3c(F)c-2ncc13] |
| 128a | 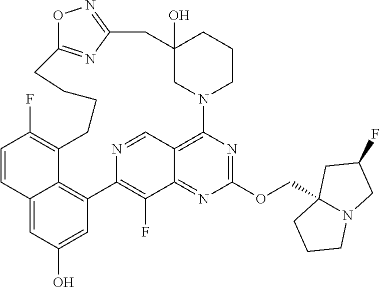 [SMILES: Oc1cc2c3c(c(F)ccc3c1)CCCCc1nc(no1)CC1(O)CCCN(C1)c1nc(OC[C@@]34CCCN3C[C@H](F)C4)nc3c(F)c-2ncc13] |
| 129 | 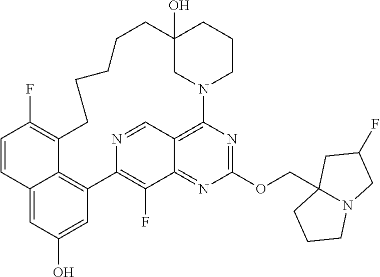 [SMILES: Oc1cc2c3c(c(F)ccc3c1)CCCCCC1(O)CCCN(C1)c1nc(OCC34CCCN3CC(F)C4)nc3c(F)c-2ncc13] |
| 129a | 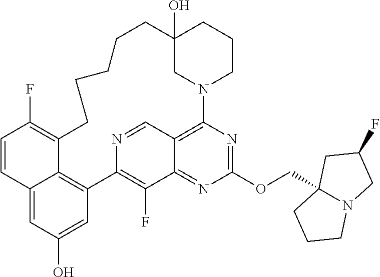 [SMILES: Oc1cc2c3c(c(F)ccc3c1)CCCCCC1(O)CCCN(C1)c1nc(OC[C@@]34CCCN3C[C@H](F)C4)nc3c(F)c-2ncc13] |
| 130 | 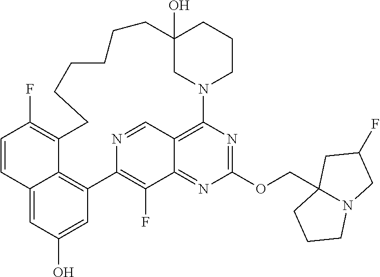 [SMILES: Oc1cc2c3c(c(F)ccc3c1)CCCCCCC1(O)CCCN(C1)c1nc(OCC34CCCN3CC(F)C4)nc3c(F)c-2ncc13] |
| 130a | 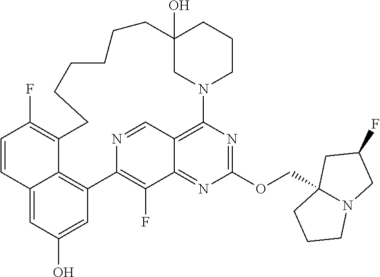 [SMILES: Oc1cc2c3c(c(F)ccc3c1)CCCCCCC1(O)CCCN(C1)c1nc(OC[C@@]34CCCN3C[C@H](F)C4)nc3c(F)c-2ncc13] |
| 131 | 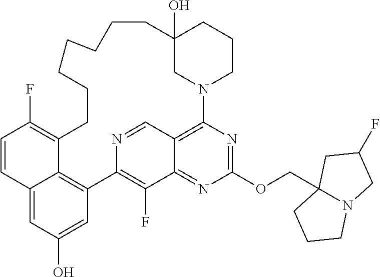 [SMILES: Oc1cc2c3c(c(F)ccc3c1)CCCCCCCC1(O)CCCN(C1)c1nc(OCC34CCCN3CC(F)C4)nc3c(F)c-2ncc13] |
| 131a | 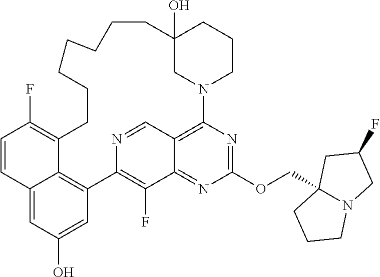 [SMILES: Oc1cc2c3c(c(F)ccc3c1)CCCCCCCC1(O)CCCN(C1)c1nc(OC[C@@]34CCCN3C[C@H](F)C4)nc3c(F)c-2ncc13] |
| 132 | 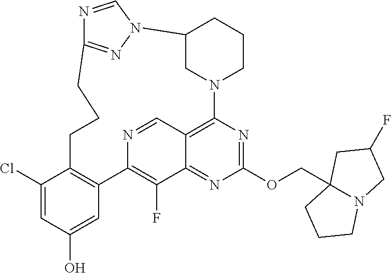 [SMILES: Oc1cc(Cl)c2c(c1)-c1ncc3c(nc(OCC45CCCN4CC(F)C5)nc3c1F)N1CCCC(C1)n1cnc(n1)CCC2] |
| 132a | 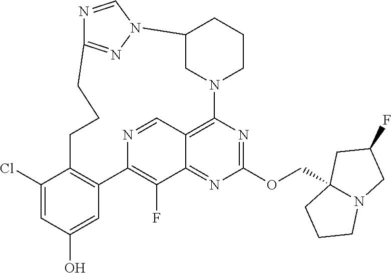 [SMILES: Oc1cc(Cl)c2c(c1)-c1ncc3c(nc(OC[C@@]45CCCN4C[C@H](F)C5)nc3c1F)N1CCCC(C1)n1cnc(n1)CCC2] |
| 133 | 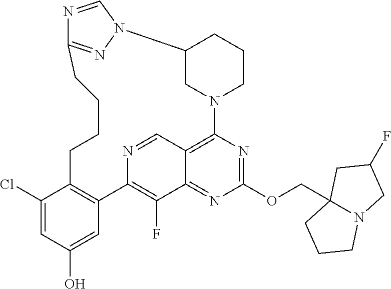 [SMILES: Oc1cc(Cl)c2c(c1)-c1ncc3c(nc(OCC45CCCN4CC(F)C5)nc3c1F)N1CCCC(C1)n1cnc(n1)CCCC2] |
| 133a | 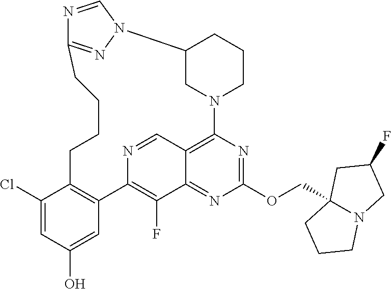 [SMILES: Oc1cc(Cl)c2c(c1)-c1ncc3c(nc(OC[C@@]45CCCN4C[C@H](F)C5)nc3c1F)N1CCCC(C1)n1cnc(n1)CCCC2] |
| 134 | 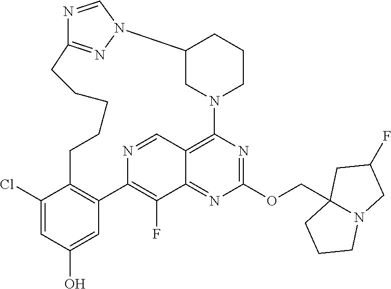 [SMILES: Oc1cc(Cl)c2c(c1)-c1ncc3c(nc(OCC45CCCN4CC(F)C5)nc3c1F)N1CCCC(C1)n1cnc(n1)CCCCC2] |
| 134a | 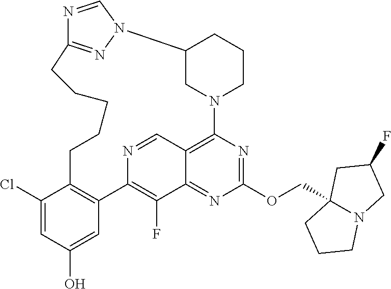 [SMILES: Oc1cc(Cl)c2c(c1)-c1ncc3c(nc(OC[C@@]45CCCN4C[C@H](F)C5)nc3c1F)N1CCCC(C1)n1cnc(n1)CCCCC2] |
| 135 | 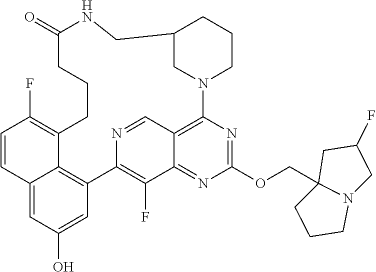 [SMILES: O=C1CCCc2c(F)ccc3cc(O)cc(c23)-c2ncc3c(nc(OCC45CCCN4CC(F)C5)nc3c2F)N2CCCC(CN1)C2] |
| 135a | 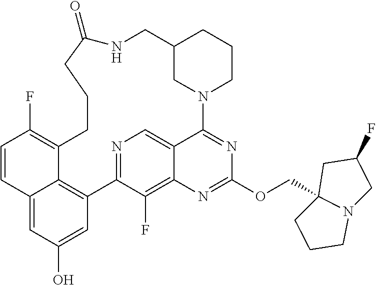 [SMILES: O=C1CCCc2c(F)ccc3cc(O)cc(c23)-c2ncc3c(nc(OC[C@@]45CCCN4C[C@H](F)C5)nc3c2F)N2CCCC(CN1)C2] |
| 136 | 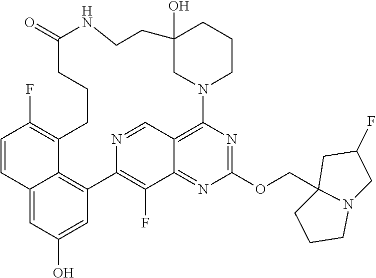 [SMILES: O=C1CCCc2c(F)ccc3cc(O)cc(c23)-c2ncc3c(nc(OCC45CCCN4CC(F)C5)nc3c2F)N2CCCC(O)(CCN1)C2] |
| 136a | 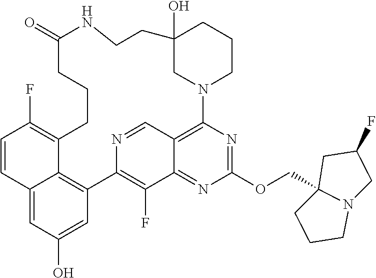 [SMILES: O=C1CCCc2c(F)ccc3cc(O)cc(c23)-c2ncc3c(nc(OC[C@@]45CCCN4C[C@H](F)C5)nc3c2F)N2CCCC(O)(CCN1)C2] |
| 137 | 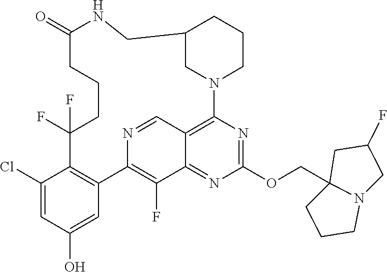 [SMILES: O=C1CCCC(F)(F)c2c(Cl)cc(O)cc2-c2ncc3c(nc(OCC45CCCN4CC(F)C5)nc3c2F)N2CCCC(CN1)C2] |
| 137a | 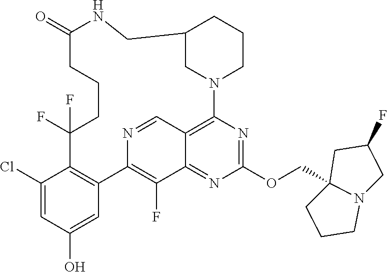 [SMILES: O=C1CCCC(F)(F)c2c(Cl)cc(O)cc2-c2ncc3c(nc(OC[C@@]45CCCN4C[C@H](F)C5)nc3c2F)N2CCCC(CN1)C2] |
| 138 | 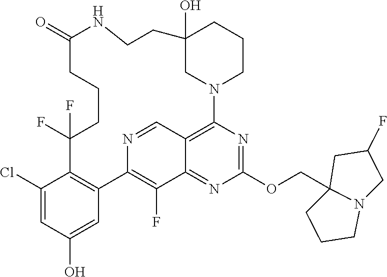 [SMILES: O=C1CCCC(F)(F)c2c(Cl)cc(O)cc2-c2ncc3c(nc(OCC45CCCN4CC(F)C5)nc3c2F)N2CCCC(O)(CCN1)C2] |
| 138a | 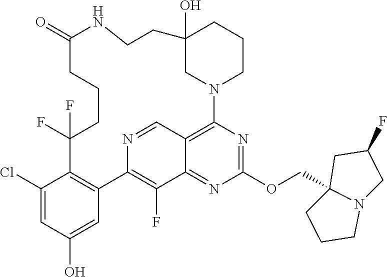 [SMILES: O=C1CCCC(F)(F)c2c(Cl)cc(O)cc2-c2ncc3c(nc(OC[C@@]45CCCN4C[C@H](F)C5)nc3c2F)N2CCCC(O)(CCN1)C2] |
| 139 | 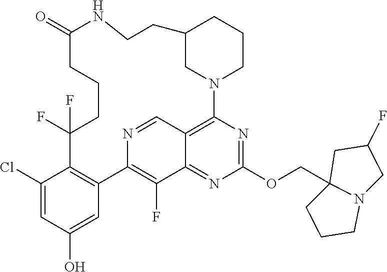 [SMILES: O=C1CCCC(F)(F)c2c(Cl)cc(O)cc2-c2ncc3c(nc(OCC45CCCN4CC(F)C5)nc3c2F)N2CCCC(CCN1)C2] |
| 139a | 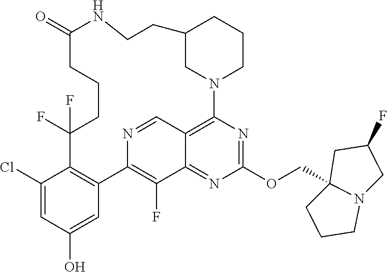 [SMILES: O=C1CCCC(F)(F)c2c(Cl)cc(O)cc2-c2ncc3c(nc(OC[C@@]45CCCN4C[C@H](F)C5)nc3c2F)N2CCCC(CCN1)C2] |
| 140 | 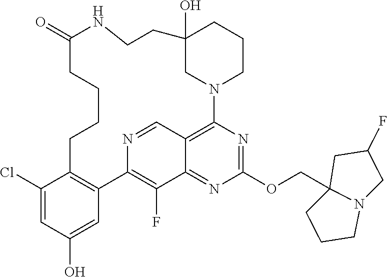 [SMILES: O=C1CCCCc2c(Cl)cc(O)cc2-c2ncc3c(nc(OCC45CCCN4CC(F)C5)nc3c2F)N2CCCC(O)(CCN1)C2] |
| 140a | 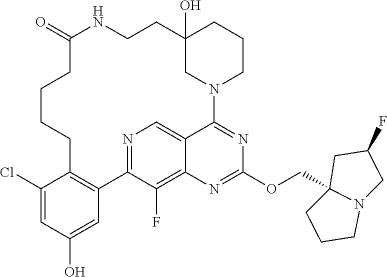 [SMILES: O=C1CCCCc2c(Cl)cc(O)cc2-c2ncc3c(nc(OC[C@@]45CCCN4C[C@H](F)C5)nc3c2F)N2CCCC(O)(CCN1)C2] |
| 140b | 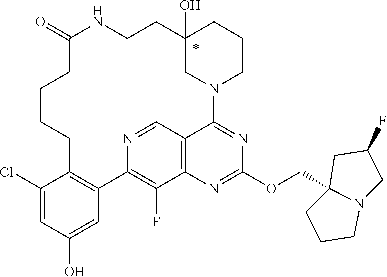 [SMILES: O=C1CCCCc2c(Cl)cc(O)cc2-c2ncc3c(nc(OC[C@@]45CCCN4C[C@H](F)C5)nc3c2F)N2CCCC(O)(CCN1)C2] |
| 140c | 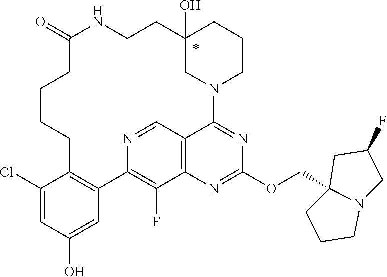 [SMILES: O=C1CCCCc2c(Cl)cc(O)cc2-c2ncc3c(nc(OC[C@@]45CCCN4C[C@H](F)C5)nc3c2F)N2CCCC(O)(CCN1)C2] |
| 141 | 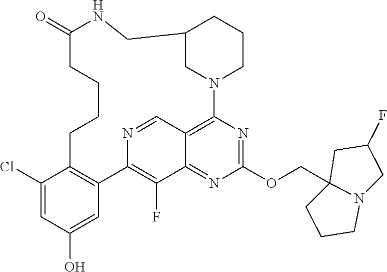 [SMILES: O=C1CCCCc2c(Cl)cc(O)cc2-c2ncc3c(nc(OCC45CCCN4CC(F)C5)nc3c2F)N2CCCC(CN1)C2] |
| 141a | 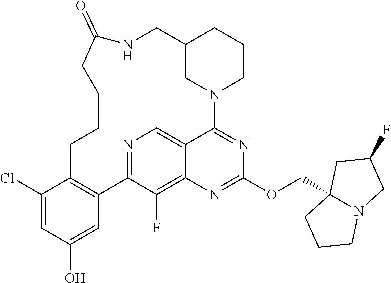 [SMILES: O=C1CCCCc2c(Cl)cc(O)cc2-c2ncc3c(nc(OC[C@@]45CCCN4C[C@H](F)C5)nc3c2F)N2CCCC(CN1)C2] |
| 143 | 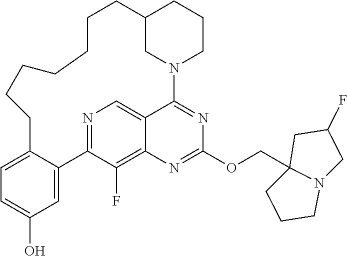 [SMILES: Oc1ccc2c(c1)-c1ncc3c(nc(OCC45CCCN4CC(F)C5)nc3c1F)N1CCCC(CCCCCCC2)C1] |
| 143a | 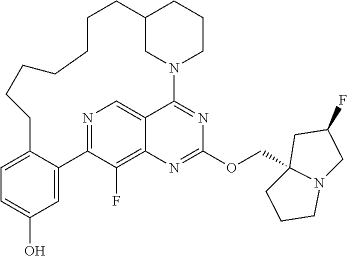 [SMILES: Oc1ccc2c(c1)-c1ncc3c(nc(OC[C@@]45CCCN4C[C@H](F)C5)nc3c1F)N1CCCC(CCCCCCC2)C1] |
| 151 | 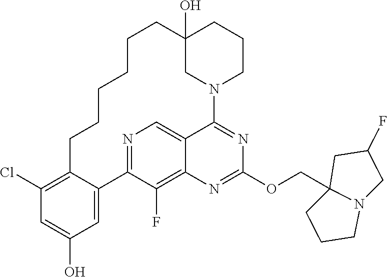 [SMILES: Oc1cc(Cl)c2c(c1)-c1ncc3c(nc(OCC45CCCN4CC(F)C5)nc3c1F)N1CCCC(O)(CCCCCC2)C1] |
| 151a | 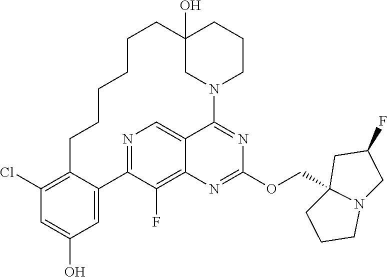 [SMILES: Oc1cc(Cl)c2c(c1)-c1ncc3c(nc(OC[C@@]45CCCN4C[C@H](F)C5)nc3c1F)N1CCCC(O)(CCCCCC2)C1] |
| 152 | 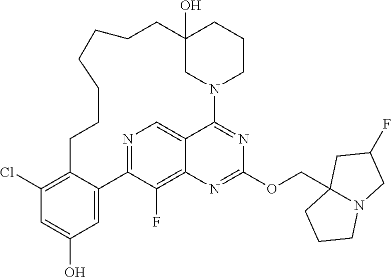 [SMILES: Oc1cc(Cl)c2c(c1)-c1ncc3c(nc(OCC45CCCN4CC(F)C5)nc3c1F)N1CCCC(O)(CCCCCCC2)C1] |
| 152a | 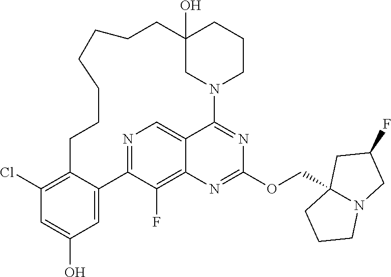 [SMILES: Oc1cc(Cl)c2c(c1)-c1ncc3c(nc(OC[C@@]45CCCN4C[C@H](F)C5)nc3c1F)N1CCCC(O)(CCCCCCC2)C1] |
| 153 | 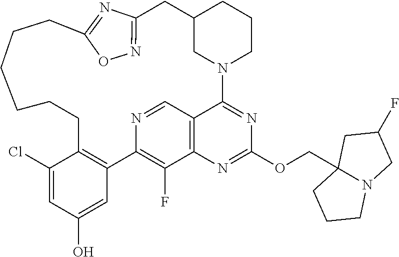 [SMILES: Oc1cc(Cl)c2c(c1)-c1ncc3c(nc(OCC45CCCN4CC(F)C5)nc3c1F)N1CCCC(Cc3noc(n3)CCCCCC2)C1] |
| 153a | 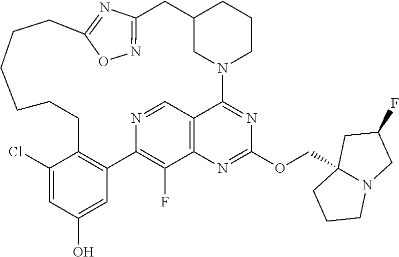 [SMILES: Oc1cc(Cl)c2c(c1)-c1ncc3c(nc(OC[C@@]45CCCN4C[C@H](F)C5)nc3c1F)N1CCCC(Cc3noc(n3)CCCCCC2)C1] |
| 154 | 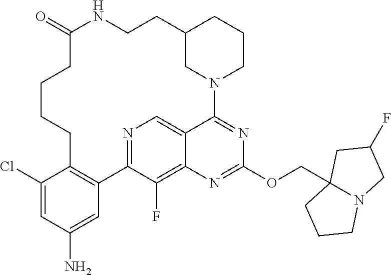 [SMILES: Nc1cc(Cl)c2c(c1)-c1ncc3c(nc(OCC45CCCN4CC(F)C5)nc3c1F)N1CCCC(CCNC(=O)CCCC2)C1] |
| 154a | 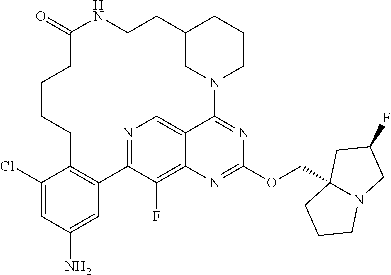 [SMILES: Nc1cc(Cl)c2c(c1)-c1ncc3c(nc(OC[C@@]45CCCN4C[C@H](F)C5)nc3c1F)N1CCCC(CCNC(=O)CCCC2)C1] |
| 155 | 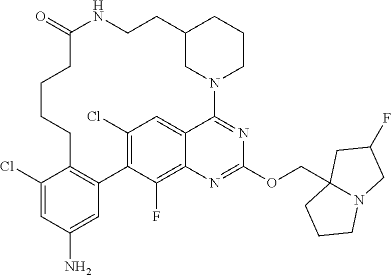 [SMILES: Nc1cc(Cl)c2c(c1)-c1c(Cl)cc3c(nc(OCC45CCCN4CC(F)C5)nc3c1F)N1CCCC(CCNC(=O)CCCC2)C1] |
| 155a | 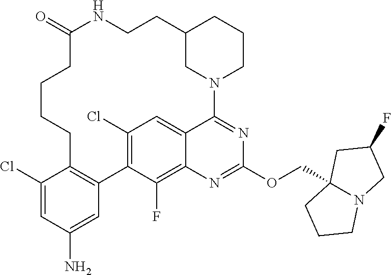 [SMILES: Nc1cc(Cl)c2c(c1)-c1c(Cl)cc3c(nc(OC[C@@]45CCCN4C[C@H](F)C5)nc3c1F)N1CCCC(CCNC(=O)CCCC2)C1] |
| 156 | 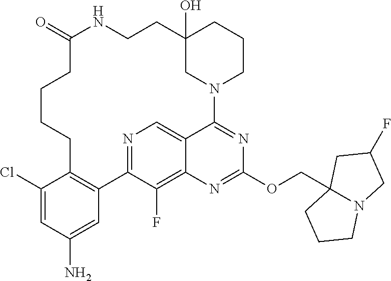 [SMILES: Nc1cc(Cl)c2c(c1)-c1ncc3c(nc(OCC45CCCN4CC(F)C5)nc3c1F)N1CCCC(O)(CCNC(=O)CCCC2)C1] |
| 156a | 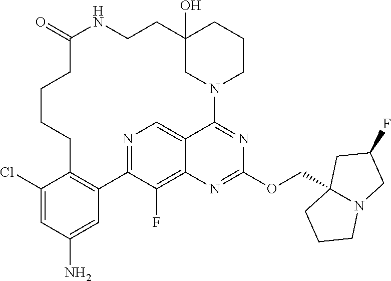 [SMILES: Nc1cc(Cl)c2c(c1)-c1ncc3c(nc(OC[C@@]45CCCN4C[C@H](F)C5)nc3c1F)N1CCCC(O)(CCNC(=O)CCCC2)C1] |
| 157 | 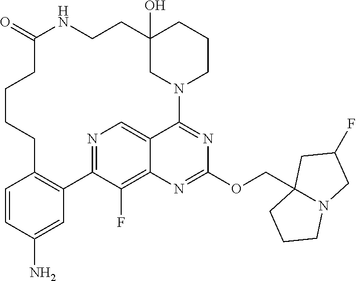 [SMILES: Nc1ccc2c(c1)-c1ncc3c(nc(OCC45CCCN4CC(F)C5)nc3c1F)N1CCCC(O)(CCNC(=O)CCCC2)C1] |
| 157a | 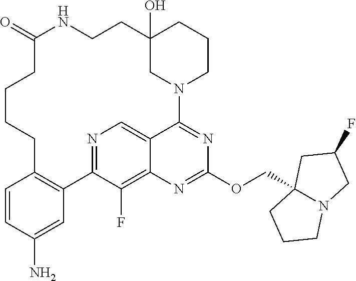 [SMILES: Nc1ccc2c(c1)-c1ncc3c(nc(OC[C@@]45CCCN4C[C@H](F)C5)nc3c1F)N1CCCC(O)(CCNC(=O)CCCC2)C1] |
| 158 | 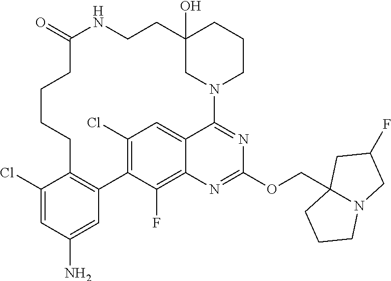 [SMILES: Nc1cc(Cl)c2c(c1)-c1c(Cl)cc3c(nc(OCC45CCCN4CC(F)C5)nc3c1F)N1CCCC(O)(CCNC(=O)CCCC2)C1] |
| 158a | 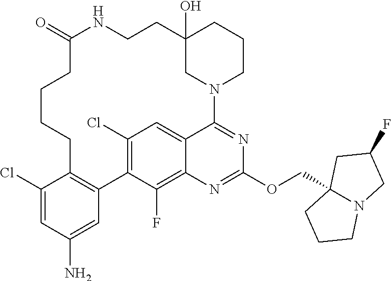 [SMILES: Nc1cc(Cl)c2c(c1)-c1c(Cl)cc3c(nc(OC[C@@]45CCCN4C[C@H](F)C5)nc3c1F)N1CCCC(O)(CCNC(=O)CCCC2)C1] |
| 159 | 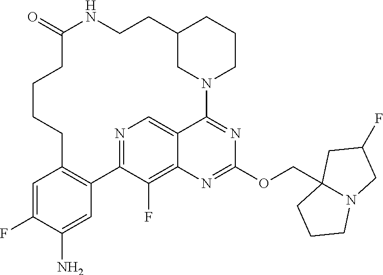 [SMILES: Nc1cc2c(cc1F)CCCCC(=O)NCCC1CCCN(C1)c1nc(OCC34CCCN3CC(F)C4)nc3c(F)c-2ncc13] |
| 159a | 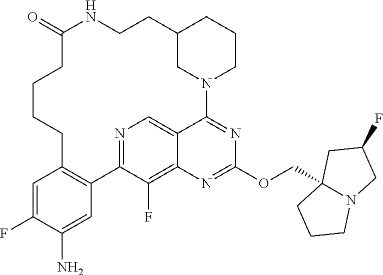 [SMILES: Nc1cc2c(cc1F)CCCCC(=O)NCCC1CCCN(C1)c1nc(OC[C@@]34CCCN3C[C@H](F)C4)nc3c(F)c-2ncc13] |
| 159b | 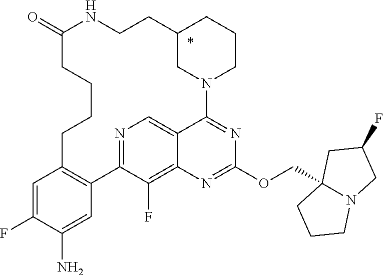 [SMILES: Nc1cc2c(cc1F)CCCCC(=O)NCCC1CCCN(C1)c1nc(OC[C@@]34CCCN3C[C@H](F)C4)nc3c(F)c-2ncc13] |
| 159c | 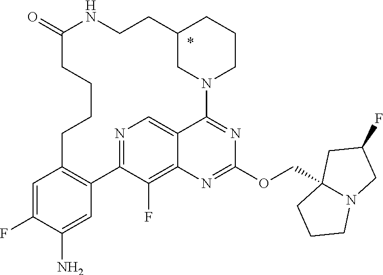 [SMILES: Nc1cc2c(cc1F)CCCCC(=O)NCCC1CCCN(C1)c1nc(OC[C@@]34CCCN3C[C@H](F)C4)nc3c(F)c-2ncc13] |
| 160 | 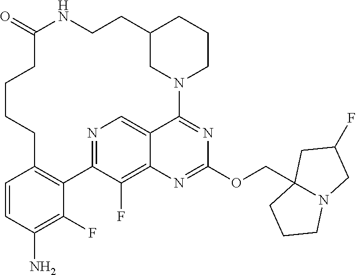 [SMILES: Nc1ccc2c(c1F)-c1ncc3c(nc(OCC45CCCN4CC(F)C5)nc3c1F)N1CCCC(CCNC(=O)CCCC2)C1] |
| 160a | 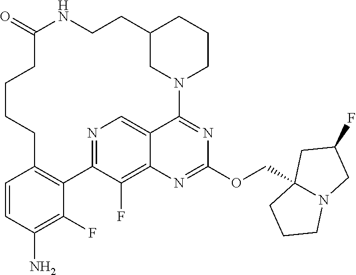 [SMILES: Nc1ccc2c(c1F)-c1ncc3c(nc(OC[C@@]45CCCN4C[C@H](F)C5)nc3c1F)N1CCCC(CCNC(=O)CCCC2)C1] |
| 161 | 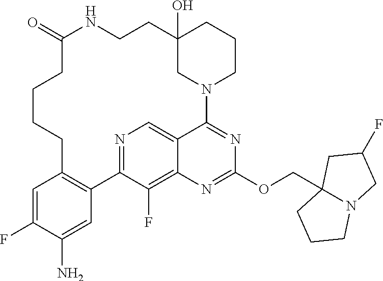 [SMILES: Nc1cc2c(cc1F)CCCCC(=O)NCCC1(O)CCCN(C1)c1nc(OCC34CCCN3CC(F)C4)nc3c(F)c-2ncc13] |
| 161a | 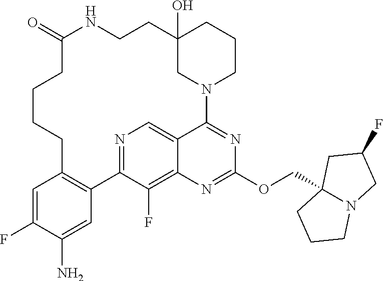 [SMILES: Nc1cc2c(cc1F)CCCCC(=O)NCCC1(O)CCCN(C1)c1nc(OC[C@@]34CCCN3C[C@H](F)C4)nc3c(F)c-2ncc13] |
| 162 | 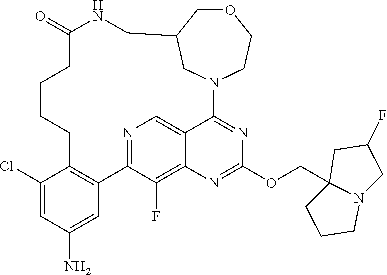 [SMILES: Nc1cc(Cl)c2c(c1)-c1ncc3c(nc(OCC45CCCN4CC(F)C5)nc3c1F)N1CCOCC(CNC(=O)CCCC2)C1] |
| 162a | 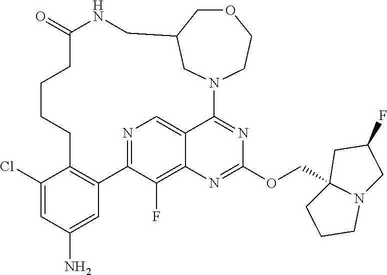 [SMILES: Nc1cc(Cl)c2c(c1)-c1ncc3c(nc(OC[C@@]45CCCN4C[C@H](F)C5)nc3c1F)N1CCOCC(CNC(=O)CCCC2)C1] |
| 163 | 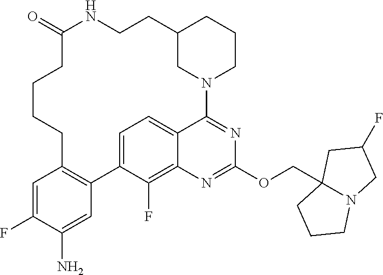 [SMILES: Nc1cc2c(cc1F)CCCCC(=O)NCCC1CCCN(C1)c1nc(OCC34CCCN3CC(F)C4)nc3c(F)c-2ccc13] |
| 163a | 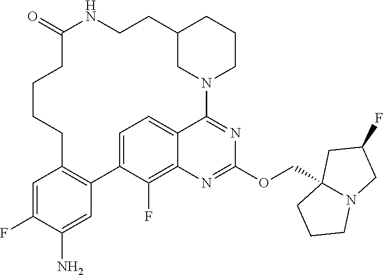 [SMILES: Nc1cc2c(cc1F)CCCCC(=O)NCCC1CCCN(C1)c1nc(OC[C@@]34CCCN3C[C@H](F)C4)nc3c(F)c-2ccc13] |
| 164 | 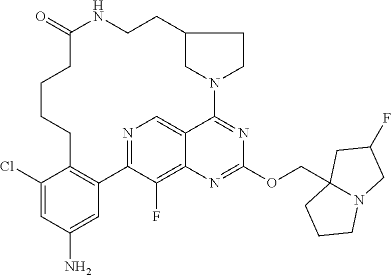 [SMILES: Nc1cc(Cl)c2c(c1)-c1ncc3c(nc(OCC45CCCN4CC(F)C5)nc3c1F)N1CCC(CCNC(=O)CCCC2)C1] |
| 164a | 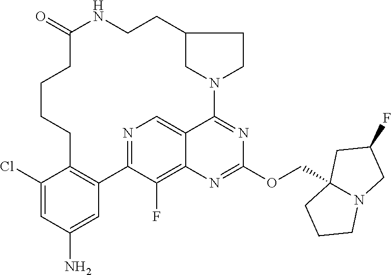 [SMILES: Nc1cc(Cl)c2c(c1)-c1ncc3c(nc(OC[C@@]45CCCN4C[C@H](F)C5)nc3c1F)N1CCC(CCNC(=O)CCCC2)C1] |
| 165 | 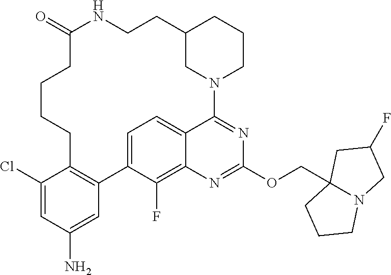 [SMILES: Nc1cc(Cl)c2c(c1)-c1ccc3c(nc(OCC45CCCN4CC(F)C5)nc3c1F)N1CCCC(CCNC(=O)CCCC2)C1] |
| 165a | 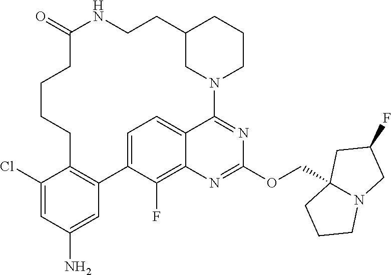 [SMILES: Nc1cc(Cl)c2c(c1)-c1ccc3c(nc(OC[C@@]45CCCN4C[C@H](F)C5)nc3c1F)N1CCCC(CCNC(=O)CCCC2)C1] |
| 166 | 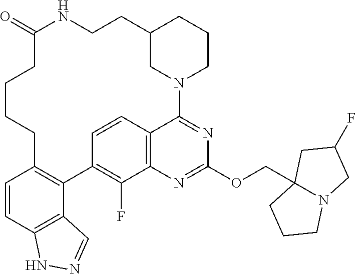 [SMILES: O=C1CCCCc2ccc3[nH]ncc3c2-c2ccc3c(nc(OCC45CCCN4CC(F)C5)nc3c2F)N2CCCC(CCN1)C2] |
| 166a | 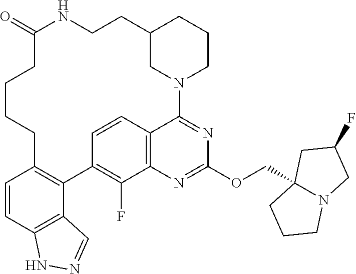 [SMILES: O=C1CCCCc2ccc3[nH]ncc3c2-c2ccc3c(nc(OC[C@@]45CCCN4C[C@H](F)C5)nc3c2F)N2CCCC(CCN1)C2] |
| 167 | 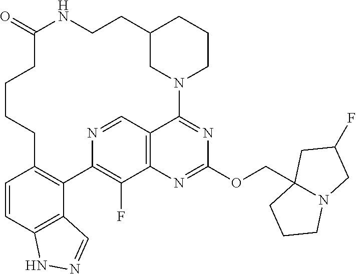 [SMILES: O=C1CCCCc2ccc3[nH]ncc3c2-c2ncc3c(nc(OCC45CCCN4CC(F)C5)nc3c2F)N2CCCC(CCN1)C2] |
| 167a | 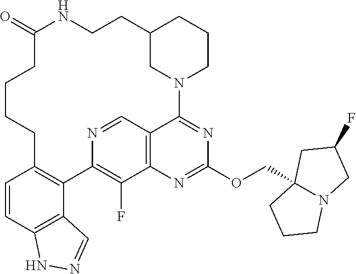 [SMILES: O=C1CCCCc2ccc3[nH]ncc3c2-c2ncc3c(nc(OC[C@@]45CCCN4C[C@H](F)C5)nc3c2F)N2CCCC(CCN1)C2] |
| 170 | 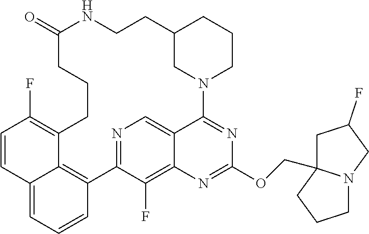 [SMILES: O=C1CCCc2c(F)ccc3cccc(c23)-c2ncc3c(nc(OCC45CCCN4CC(F)C5)nc3c2F)N2CCCC(CCN1)C2] |
| 170a | 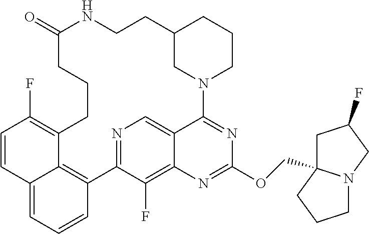 [SMILES: O=C1CCCc2c(F)ccc3cccc(c23)-c2ncc3c(nc(OC[C@@]45CCCN4C[C@H](F)C5)nc3c2F)N2CCCC(CCN1)C2] |
| 171 | 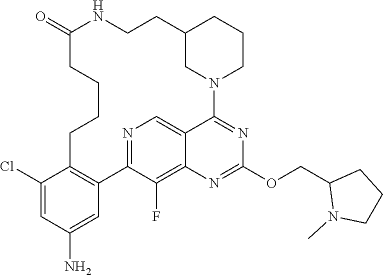 [SMILES: CN1CCCC1COc1nc2c3cnc(c(F)c3n1)-c1cc(N)cc(Cl)c1CCCCC(=O)NCCC1CCCN2C1] |
| 171a | 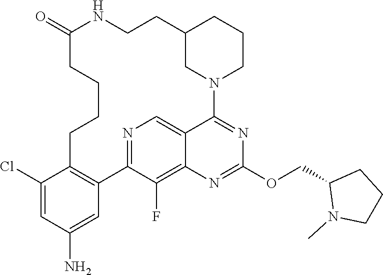 [SMILES: CN1CCC[C@H]1COc1nc2c3cnc(c(F)c3n1)-c1cc(N)cc(Cl)c1CCCCC(=O)NCCC1CCCN2C1] |
| 172 | 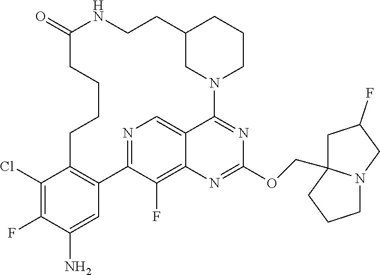 [SMILES: Nc1cc2c(c(Cl)c1F)CCCCC(=O)NCCC1CCCN(C1)c1nc(OCC34CCCN3CC(F)C4)nc3c(F)c-2ncc13] |
| 172a | 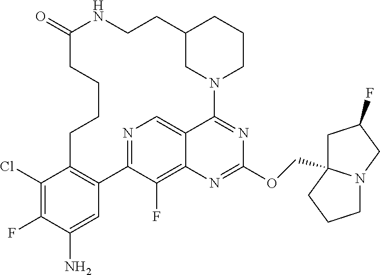 [SMILES: Nc1cc2c(c(Cl)c1F)CCCCC(=O)NCCC1CCCN(C1)c1nc(OC[C@@]34CCCN3C[C@H](F)C4)nc3c(F)c-2ncc13] |
| 173 | 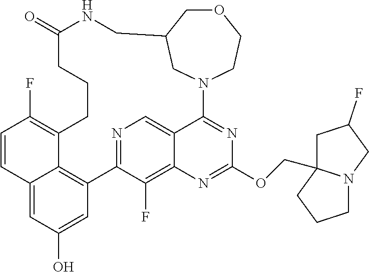 [SMILES: O=C1CCCc2c(F)ccc3cc(O)cc(c23)-c2ncc3c(nc(OCC45CCCN4CC(F)C5)nc3c2F)N2CCOCC(CN1)C2] |
| 173a | 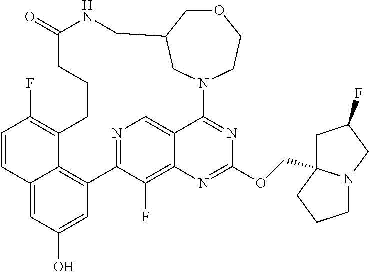 [SMILES: O=C1CCCc2c(F)ccc3cc(O)cc(c23)-c2ncc3c(nc(OC[C@@]45CCCN4C[C@H](F)C5)nc3c2F)N2CCOCC(CN1)C2] |
| 174 | 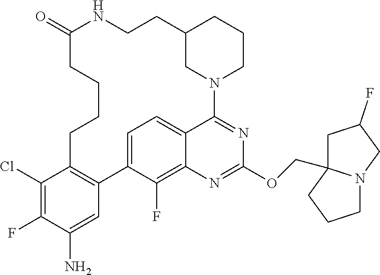 [SMILES: Nc1cc2c(c(Cl)c1F)CCCCC(=O)NCCC1CCCN(C1)c1nc(OCC34CCCN3CC(F)C4)nc3c(F)c-2ccc13] |
| 174a | 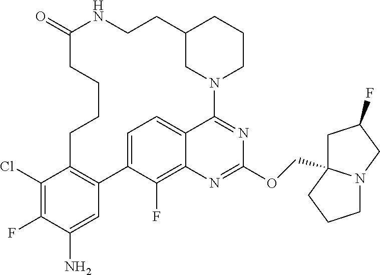 [SMILES: Nc1cc2c(c(Cl)c1F)CCCCC(=O)NCCC1CCCN(C1)c1nc(OC[C@@]34CCCN3C[C@H](F)C4)nc3c(F)c-2ccc13] |
| 175 | 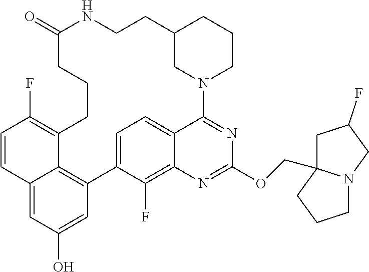 [SMILES: O=C1CCCc2c(F)ccc3cc(O)cc(c23)-c2ccc3c(nc(OCC45CCCN4CC(F)C5)nc3c2F)N2CCCC(CCN1)C2] |
| 175a | 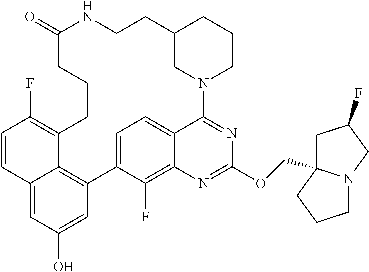 [SMILES: O=C1CCCc2c(F)ccc3cc(O)cc(c23)-c2ccc3c(nc(OC[C@@]45CCCN4C[C@H](F)C5)nc3c2F)N2CCCC(CCN1)C2] |
| 176 | 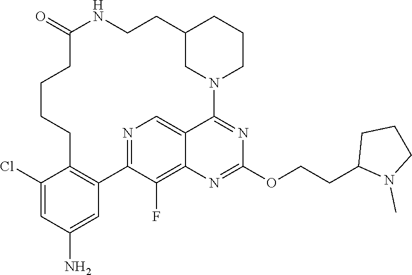 [SMILES: CN1CCCC1CCOc1nc2c3cnc(c(F)c3n1)-c1cc(N)cc(Cl)c1CCCCC(=O)NCCC1CCCN2C1] |
| 176a | 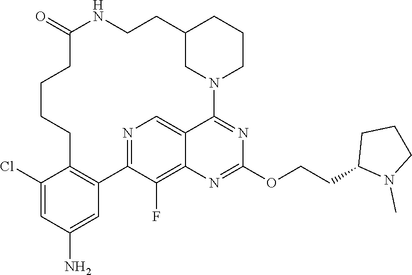 [SMILES: CN1CCC[C@H]1CCOc1nc2c3cnc(c(F)c3n1)-c1cc(N)cc(Cl)c1CCCCC(=O)NCCC1CCCN2C1] |
| 177 | 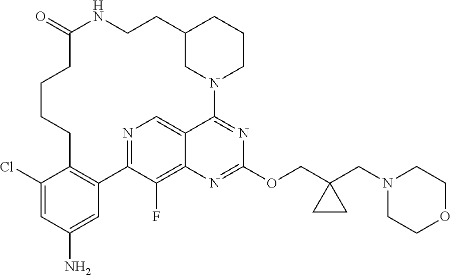 [SMILES: Nc1cc(Cl)c2c(c1)-c1ncc3c(nc(OCC4(CN5CCOCC5)CC4)nc3c1F)N1CCCC(CCNC(=O)CCCC2)C1] |
| 178 | 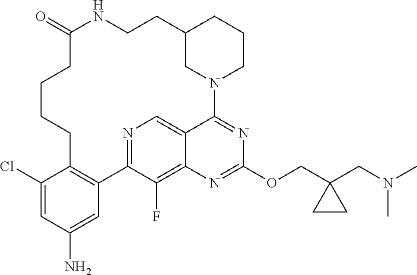 [SMILES: CN(C)CC1(COc2nc3c4cnc(c(F)c4n2)-c2cc(N)cc(Cl)c2CCCCC(=O)NCCC2CCCN3C2)CC1] |
| 179 | 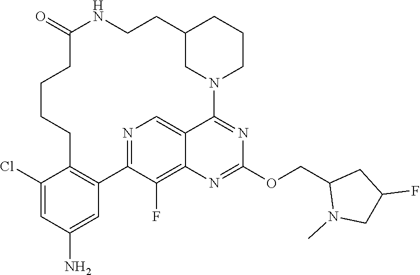 [SMILES: CN1CC(F)CC1COc1nc2c3cnc(c(F)c3n1)-c1cc(N)cc(Cl)c1CCCCC(=O)NCCC1CCCN2C1] |
| 179a | 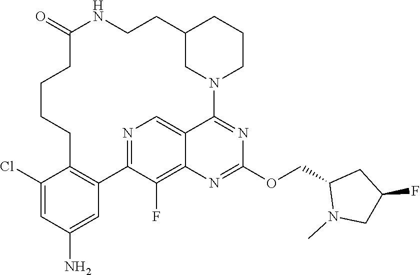 [SMILES: CN1C[C@H](F)C[C@H]1COc1nc2c3cnc(c(F)c3n1)-c1cc(N)cc(Cl)c1CCCCC(=O)NCCC1CCCN2C1] |
| 180 | 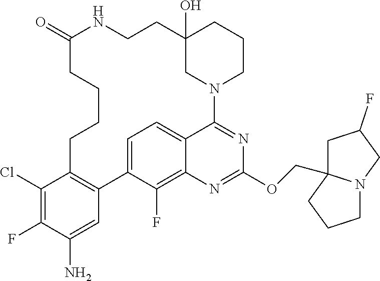 [SMILES: Nc1cc2c(c(Cl)c1F)CCCCC(=O)NCCC1(O)CCCN(C1)c1nc(OCC34CCCN3CC(F)C4)nc3c(F)c-2ccc13] |
| 180a | 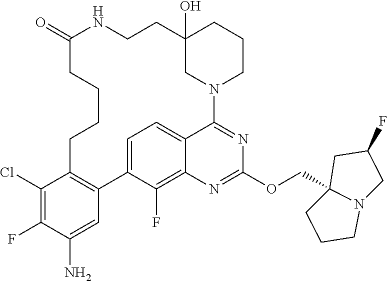 [SMILES: Nc1cc2c(c(Cl)c1F)CCCCC(=O)NCCC1(O)CCCN(C1)c1nc(OC[C@@]34CCCN3C[C@H](F)C4)nc3c(F)c-2ccc13] |
| 181 | 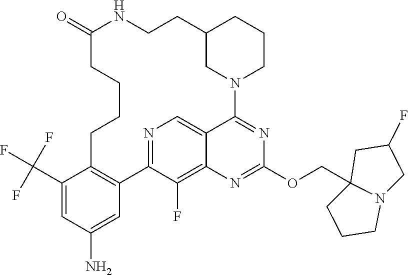 [SMILES: Nc1cc2c(c(C(F)(F)F)c1)CCCCC(=O)NCCC1CCCN(C1)c1nc(OCC34CCCN3CC(F)C4)nc3c(F)c-2ncc13] |
| 181a | 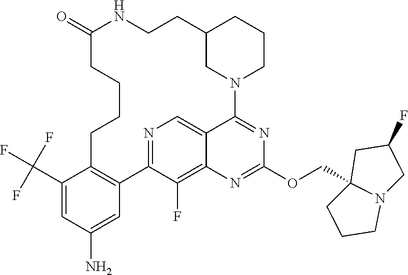 [SMILES: Nc1cc2c(c(C(F)(F)F)c1)CCCCC(=O)NCCC1CCCN(C1)c1nc(OC[C@@]34CCCN3C[C@H](F)C4)nc3c(F)c-2ncc13] |
| 182 | 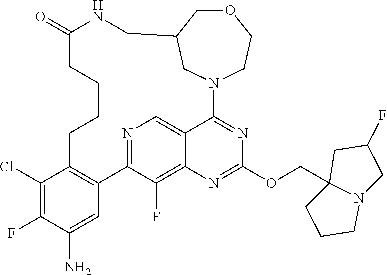 [SMILES: Nc1cc2c(c(Cl)c1F)CCCCC(=O)NCC1COCCN(C1)c1nc(OCC34CCCN3CC(F)C4)nc3c(F)c-2ncc13] |
| 182a | 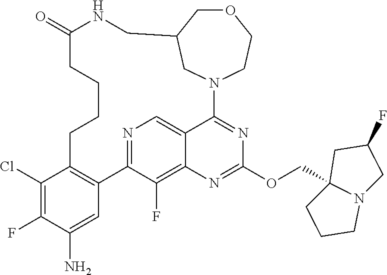 [SMILES: Nc1cc2c(c(Cl)c1F)CCCCC(=O)NCC1COCCN(C1)c1nc(OC[C@@]34CCCN3C[C@H](F)C4)nc3c(F)c-2ncc13] |
| 183 | 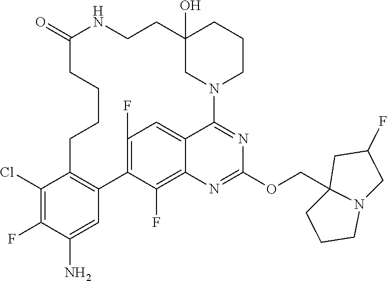 [SMILES: Nc1cc2c(c(Cl)c1F)CCCCC(=O)NCCC1(O)CCCN(C1)c1nc(OCC34CCCN3CC(F)C4)nc3c(F)c-2c(F)cc13] |
| 183a | 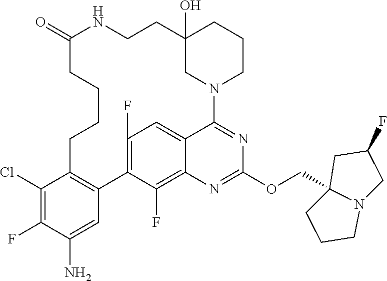 [SMILES: Nc1cc2c(c(Cl)c1F)CCCCC(=O)NCCC1(O)CCCN(C1)c1nc(OC[C@@]34CCCN3C[C@H](F)C4)nc3c(F)c-2c(F)cc13] |
| 184 | 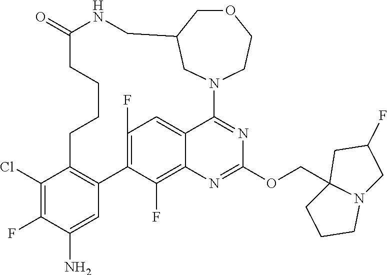 [SMILES: Nc1cc2c(c(Cl)c1F)CCCCC(=O)NCC1COCCN(C1)c1nc(OCC34CCCN3CC(F)C4)nc3c(F)c-2c(F)cc13] |
| 184a | 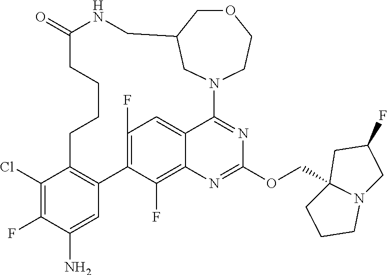 [SMILES: Nc1cc2c(c(Cl)c1F)CCCCC(=O)NCC1COCCN(C1)c1nc(OC[C@@]34CCCN3C[C@H](F)C4)nc3c(F)c-2c(F)cc13] |
| 185 | 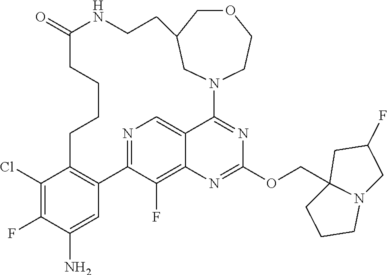 [SMILES: Nc1cc2c(c(Cl)c1F)CCCCC(=O)NCCC1COCCN(C1)c1nc(OCC34CCCN3CC(F)C4)nc3c(F)c-2ncc13] |
| 185a | 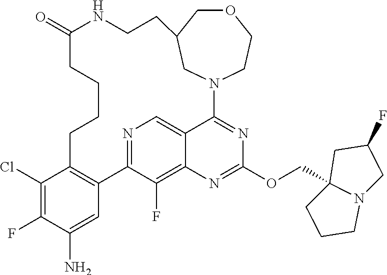 [SMILES: Nc1cc2c(c(Cl)c1F)CCCCC(=O)NCCC1COCCN(C1)c1nc(OC[C@@]34CCCN3C[C@H](F)C4)nc3c(F)c-2ncc13] |
| 186 | 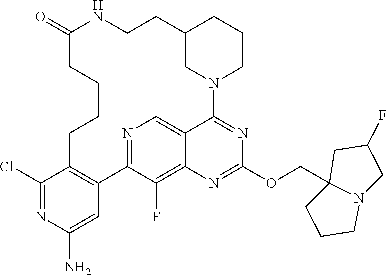 [SMILES: Nc1cc2c(c(Cl)n1)CCCCC(=O)NCCC1CCCN(C1)c1nc(OCC34CCCN3CC(F)C4)nc3c(F)c-2ncc13] |
| 186a | 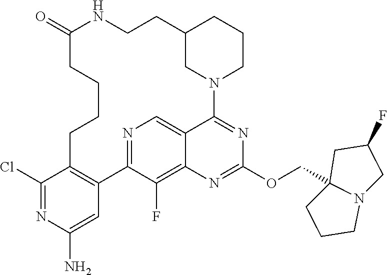 |
| 187 | 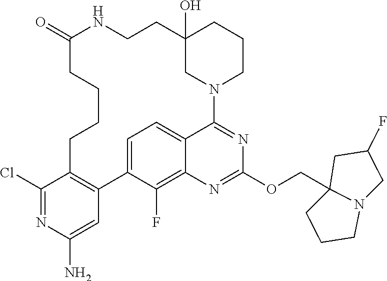 [SMILES: Nc1cc2c(c(Cl)n1)CCCCC(=O)NCCC1(O)CCCN(C1)c1nc(OCC34CCCN3CC(F)C4)nc3c(F)c-2ccc13] |
| 187a | 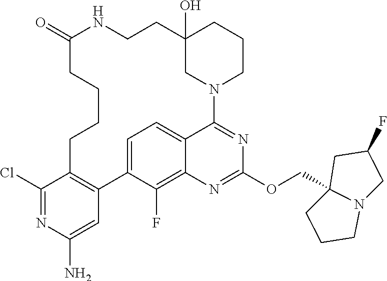 [SMILES: Nc1cc2c(c(Cl)n1)CCCCC(=O)NCCC1(O)CCCN(C1)c1nc(OC[C@@]34CCCN3C[C@H](F)C4)nc3c(F)c-2ccc13] |
| 188 | 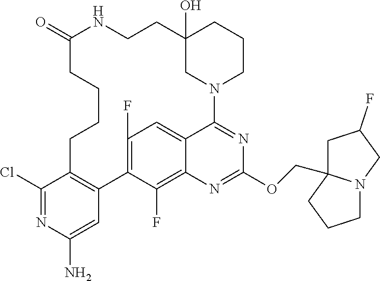 [SMILES: Nc1cc2c(c(Cl)n1)CCCCC(=O)NCCC1(O)CCCN(C1)c1nc(OCC34CCCN3CC(F)C4)nc3c(F)c-2c(F)cc13] |
| 188a | 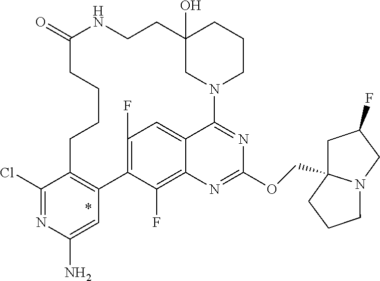 [SMILES: Nc1cc2c(c(Cl)n1)CCCCC(=O)NCCC1(O)CCCN(C1)c1nc(OC[C@@]34CCCN3C[C@H](F)C4)nc3c(F)c-2c(F)cc13] |
| 188b | 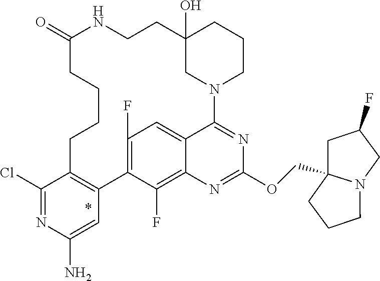 [SMILES: Nc1cc2c(c(Cl)n1)CCCCC(=O)NCCC1(O)CCCN(C1)c1nc(OC[C@@]34CCCN3C[C@H](F)C4)nc3c(F)c-2c(F)cc13] |
| 189 | 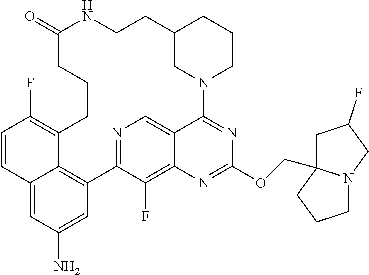 [SMILES: Nc1cc2c3c(c(F)ccc3c1)CCCC(=O)NCCC1CCCN(C1)c1nc(OCC34CCCN3CC(F)C4)nc3c(F)c-2ncc13] |
| 189a | 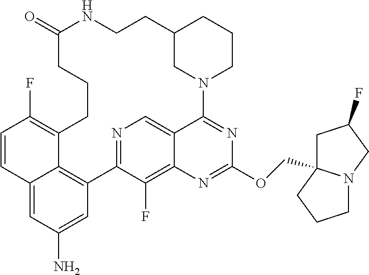 [SMILES: Nc1cc2c3c(c(F)ccc3c1)CCCC(=O)NCCC1CCCN(C1)c1nc(OC[C@@]34CCCN3C[C@H](F)C4)nc3c(F)c-2ncc13] |
| 190 | 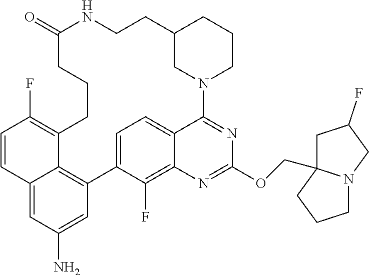 [SMILES: Nc1cc2c3c(c(F)ccc3c1)CCCC(=O)NCCC1CCCN(C1)c1nc(OCC34CCCN3CC(F)C4)nc3c(F)c-2ccc13] |
| 190a | 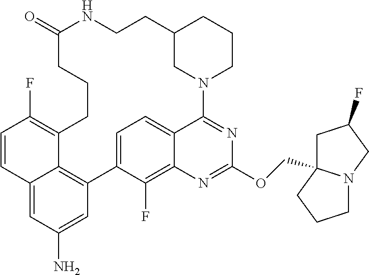 [SMILES: Nc1cc2c3c(c(F)ccc3c1)CCCC(=O)NCCC1CCCN(C1)c1nc(OC[C@@]34CCCN3C[C@H](F)C4)nc3c(F)c-2ccc13] |
| 191 | 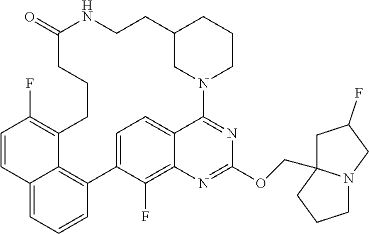 [SMILES: O=C1CCCc2c(F)ccc3cccc(c23)-c2ccc3c(nc(OCC45CCCN4CC(F)C5)nc3c2F)N2CCCC(CCN1)C2] |
| 191a | 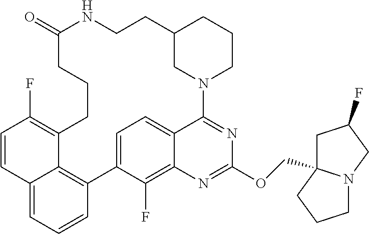 [SMILES: O=C1CCCc2c(F)ccc3cccc(c23)-c2ccc3c(nc(OC[C@@]45CCCN4C[C@H](F)C5)nc3c2F)N2CCCC(CCN1)C2] |
| 192 | 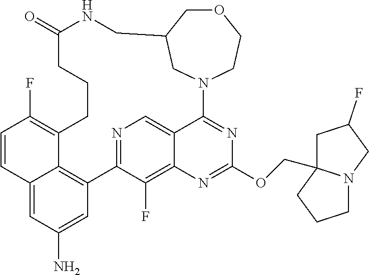 [SMILES: Nc1cc2c3c(c(F)ccc3c1)CCCC(=O)NCC1COCCN(C1)c1nc(OCC34CCCN3CC(F)C4)nc3c(F)c-2ncc13] |
| 192a | 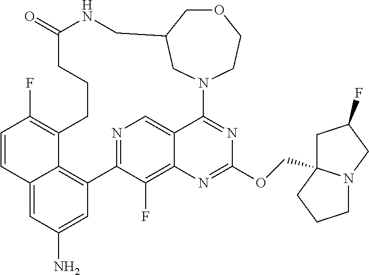 [SMILES: Nc1cc2c3c(c(F)ccc3c1)CCCC(=O)NCC1COCCN(C1)c1nc(OC[C@@]34CCCN3C[C@H](F)C4)nc3c(F)c-2ncc13] |
| 193 | 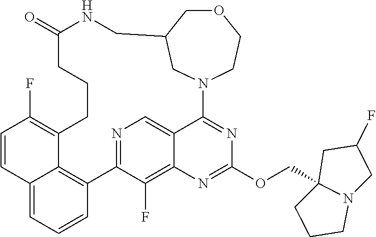 [SMILES: O=C1CCCc2c(F)ccc3cccc(c23)-c2ncc3c(nc(OC[C@@]45CCCN4CC(F)C5)nc3c2F)N2CCOCC(CN1)C2] |
| 193a | 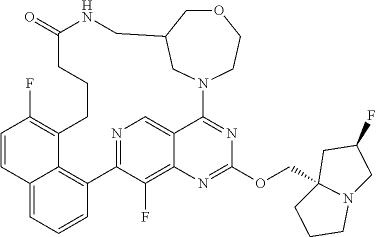 [SMILES: O=C1CCCc2c(F)ccc3cccc(c23)-c2ncc3c(nc(OC[C@@]45CCCN4C[C@H](F)C5)nc3c2F)N2CCOCC(CN1)C2] |
| 194 | 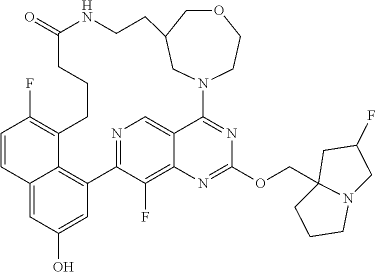 [SMILES: O=C1CCCc2c(F)ccc3cc(O)cc(c23)-c2ncc3c(nc(OCC45CCCN4CC(F)C5)nc3c2F)N2CCOCC(CCN1)C2] |
| 194a | 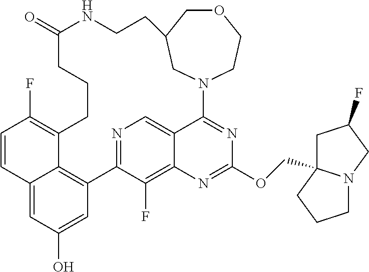 [SMILES: O=C1CCCc2c(F)ccc3cc(O)cc(c23)-c2ncc3c(nc(OC[C@@]45CCCN4C[C@H](F)C5)nc3c2F)N2CCOCC(CCN1)C2] |
| 195 | 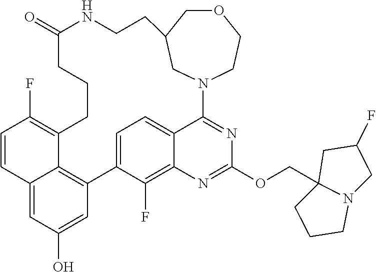 [SMILES: O=C1CCCc2c(F)ccc3cc(O)cc(c23)-c2ccc3c(nc(OCC45CCCN4CC(F)C5)nc3c2F)N2CCOCC(CCN1)C2] |
| 195a | 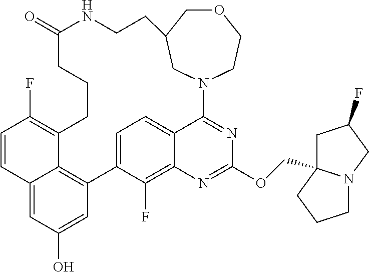 [SMILES: O=C1CCCc2c(F)ccc3cc(O)cc(c23)-c2ccc3c(nc(OC[C@@]45CCCN4C[C@H](F)C5)nc3c2F)N2CCOCC(CCN1)C2] |
| 196 | 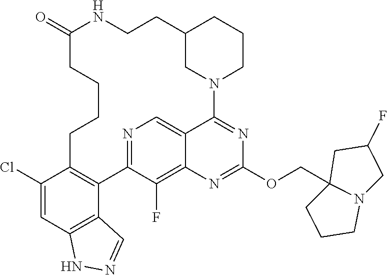 [SMILES: O=C1CCCCc2c(Cl)cc3[nH]ncc3c2-c2ncc3c(nc(OCC45CCCN4CC(F)C5)nc3c2F)N2CCCC(CCN1)C2] |
| 196a | 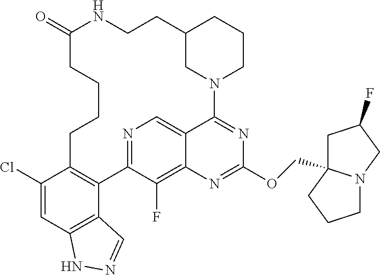 [SMILES: O=C1CCCCc2c(Cl)cc3[nH]ncc3c2-c2ncc3c(nc(OC[C@@]45CCCN4C[C@H](F)C5)nc3c2F)N2CCCC(CCN1)C2] |
| 197 | 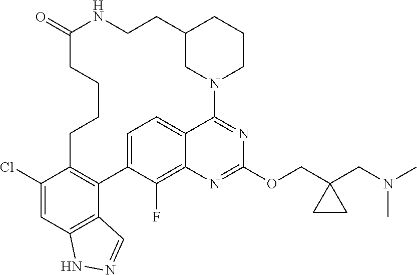 [SMILES: CN(C)CC1(COc2nc3c4ccc(c(F)c4n2)-c2c(c(Cl)cc4[nH]ncc24)CCCCC(=O)NCCC2CCCN3C2)CC1] |
| 197a | 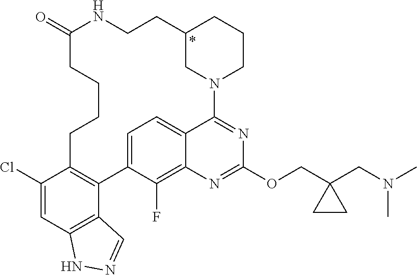 [SMILES: CN(C)CC1(COc2nc3c4ccc(c(F)c4n2)-c2c(c(Cl)cc4[nH]ncc24)CCCCC(=O)NCCC2CCCN3C2)CC1] |
| 197b | 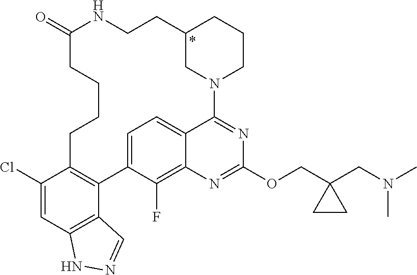 [SMILES: CN(C)CC1(COc2nc3c4ccc(c(F)c4n2)-c2c(c(Cl)cc4[nH]ncc24)CCCCC(=O)NCCC2CCCN3C2)CC1] |
| 198 | 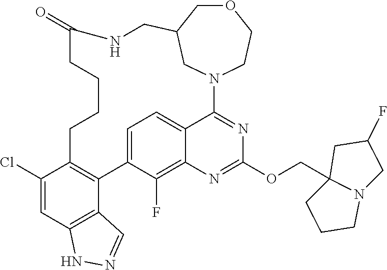 [SMILES: O=C1CCCCc2c(Cl)cc3[nH]ncc3c2-c2ccc3c(nc(OCC45CCCN4CC(F)C5)nc3c2F)N2CCOCC(CN1)C2] |
| 198a | 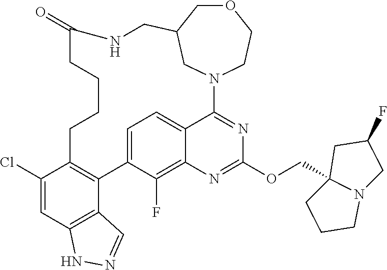 [SMILES: O=C1CCCCc2c(Cl)cc3[nH]ncc3c2-c2ccc3c(nc(OC[C@@]45CCCN4C[C@H](F)C5)nc3c2F)N2CCOCC(CN1)C2] |
| 199 | 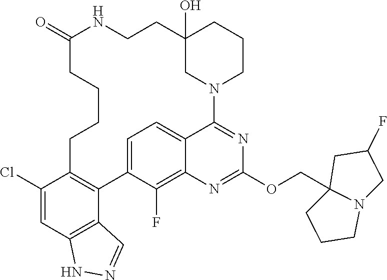 [SMILES: O=C1CCCCc2c(Cl)cc3[nH]ncc3c2-c2ccc3c(nc(OCC45CCCN4CC(F)C5)nc3c2F)N2CCCC(O)(CCN1)C2] |
| 199a | 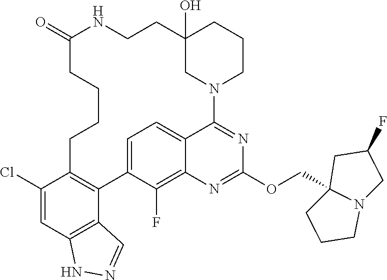 [SMILES: O=C1CCCCc2c(Cl)cc3[nH]ncc3c2-c2ccc3c(nc(OC[C@@]45CCCN4C[C@H](F)C5)nc3c2F)N2CCCC(O)(CCN1)C2] |
| 200 | 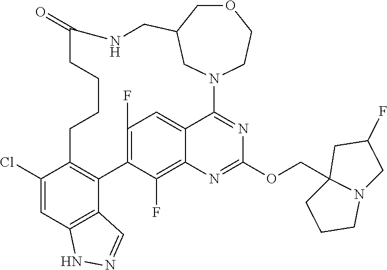 [SMILES: O=C1CCCCc2c(Cl)cc3[nH]ncc3c2-c2c(F)cc3c(nc(OCC45CCCN4CC(F)C5)nc3c2F)N2CCOCC(CN1)C2] |
| 200a | 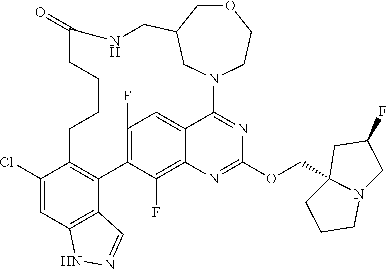 [SMILES: O=C1CCCCc2c(Cl)cc3[nH]ncc3c2-c2c(F)cc3c(nc(OC[C@@]45CCCN4C[C@H](F)C5)nc3c2F)N2CCOCC(CN1)C2] |
| 201 | 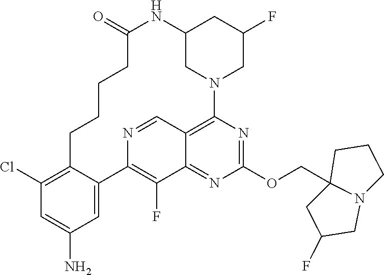 [SMILES: Nc1cc(Cl)c2c(c1)-c1ncc3c(nc(OCC45CCCN4CC(F)C5)nc3c1F)N1CC(F)CC(C1)NC(=O)CCCC2] |
| 201a | 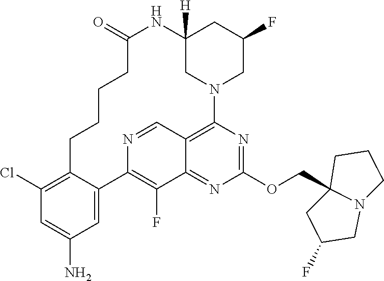 [SMILES: Nc1cc(Cl)c2c(c1)-c1ncc3c(nc(OC[C@@]45CCCN4C[C@H](F)C5)nc3c1F)N1C[C@H](F)C[C@H](C1)NC(=O)CCCC2] |
| 202 | 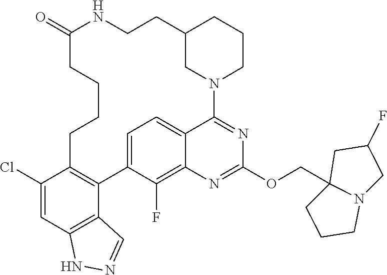 [SMILES: O=C1CCCCc2c(Cl)cc3[nH]ncc3c2-c2ccc3c(nc(OCC45CCCN4CC(F)C5)nc3c2F)N2CCCC(CCN1)C2] |
| 202a | 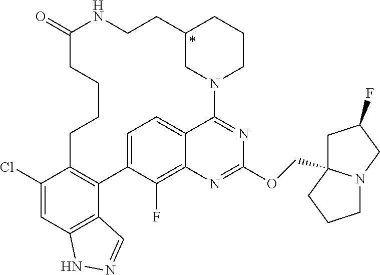 [SMILES: O=C1CCCCc2c(Cl)cc3[nH]ncc3c2-c2ccc3c(nc(OC[C@@]45CCCN4C[C@H](F)C5)nc3c2F)N2CCCC(CCN1)C2] |
| 202b | 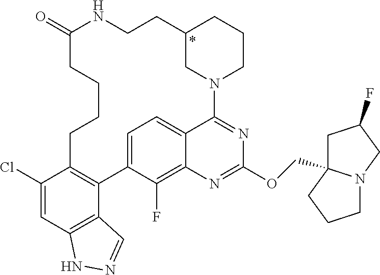 [SMILES: O=C1CCCCc2c(Cl)cc3[nH]ncc3c2-c2ccc3c(nc(OC[C@@]45CCCN4C[C@H](F)C5)nc3c2F)N2CCCC(CCN1)C2] |
| 203 | 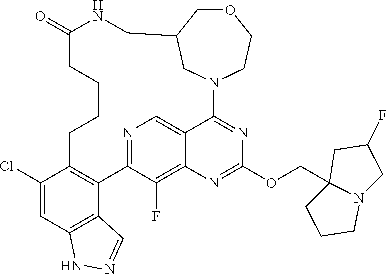 [SMILES: O=C1CCCCc2c(Cl)cc3[nH]ncc3c2-c2ncc3c(nc(OCC45CCCN4CC(F)C5)nc3c2F)N2CCOCC(CN1)C2] |
| 203a | 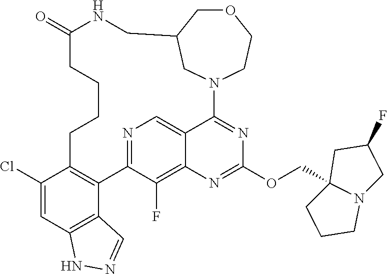 [SMILES: O=C1CCCCc2c(Cl)cc3[nH]ncc3c2-c2ncc3c(nc(OC[C@@]45CCCN4C[C@H](F)C5)nc3c2F)N2CCOCC(CN1)C2] |

Note:
When a stereogenic center (e.g., a chiral carbon or a center of axial chirality) is denoted with a ″*,″ this stereogenic center has been resolved, but its absolute configuration is not assigned.

Exemplary compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)) also include those depicted in Table C1 of U.S. Provisional Application Ser. No. 63/398,737, filed on Aug. 17, 2022; Table C1 of U.S. Provisional Application Ser. No. 63/426,885, filed on Nov. 21, 2022; Table C1 of U.S. Provisional Application Ser. No. 63/456,316, filed on Mar. 31, 2023; and Table C1 of U.S. Provisional Application Ser. No. 63/526,503, filed on Jul. 13, 2023; or pharmaceutically acceptable salts thereof, wherein each Table C1 is incorporated herein by reference in its entirety.

Exemplary compounds of Formula (A) include compounds: 101, 101a, 102, 102a, 103, 103a, 104, 104a, 105, 105a, 106, 106a, 107, 107a, 108, 108a, 109, 109a, 110, 110a, 111, 111a, 112,112a, 113,113a, 114,114a, 115,115a, 115b, 115c, 116,116a, 117,117a, 118,118a, 119, 119a, 120, 120a, 121, 121a, 122, 122a, 123, 123a, 124, 124a, 125, 125a, 126, 126a, 127, 127a, 128, 128a, 129, 129a, 130, 130a, 131, 131a, 132, 132a, 133, 133a, 134, 134a, 135, 135a, 136, 136a, 137, 137a, 138, 138a, 139, 139a, 140, 140a, 140b, 140c, 141, 141a, 143, 143a, 151, 151a, 152,152a, 153,153a, 154,154a, 155,155a, 156,156a, 157,157a, 158,158a, 159,159a, 159b, 159c, 160, 160a, 161, 161a, 162, 162a, 163, 163a, 164, 164a, 165, 165a, 166, 166a, 167, 167a, 170, 170a, 171, 171a, 172, 172a, 173, 173a, 174, 174a, 175, 175a, 176, 176a, 177, 178, 179, 179a, 180, 180a, 181, 181a, 182, 182a, 183, 183a, 184, 184a, 185, 185a, 186, 186a, 187, 187a, 188, 188a, 188b, 189, 189a, 190, 190a, 191, 191a, 192, 192a, 193, 193a, 194, 194a, 195, 195a, 196, 196a, 197, 197a, 197b, 198, 198a, 199, 199a, 200, 200a, 201, 201a, 202, 202a, 202b, 203, and 203a.

Exemplary compounds of Formula (Aa) include compounds: 113, 113a, 114, 114a, 115,115a, 115b, 115c, 117,117a, 118,118a, 135,135a, 136,136a, 137,137a, 138,138a, 139, 139a, 140, 140a, 140b, 140c, 141, 141a, 154, 154a, 155, 155a, 156, 156a, 158, 158a, 162, 162a, 165, 165a, 166, 166a, 167, 167a, 170, 170a, 171, 171a, 173, 173a, 175, 175a, 177, 179, 179a, 181, 181a, 189, 189a, 190, 190a, 191, 191a, 192, 192a, 193, 193a, 194, 194a, 195, 195a, 196, 196a, 197a, 197b, 198, 198a, 199, 199a, 200, 200a, 201, 201a, 202, 202a, 202b, 203, and 203a.

Exemplary compounds of Formula (I) include compounds: 101, 101a, 102, 102a, 103, 103a, 104,104a, 105,105a, 106,106a, 107,107a, 108,108a, 109,109a, 110, 110a, 111, 111a, 112,112a, 113,113a, 114,114a, 115,115a, 115b, 115c, 116,116a, 117,117a, 118,118a, 119, 119a, 120, 120a, 121, 121a, 122, 122a, 123, 123a, 124, 124a, 125, 125a, 126, 126a, 127, 127a, 128, 128a, 129, 129a, 130, 130a, 131, 131a, 132, 132a, 133, 133a, 134, 134a, 135, 135a, 136, 136a, 137, 137a, 138, 138a, 139, 139a, 140, 140a, 140b, 140c, 141, 141a, 143, 143a, 151, 151a, 152, 152a, 153, 153a, 154, 154a, 156, 156a, 157, 157a, 159, 159a, 159b, 159c, 160, 160a, 161, 161a, 162, 162a, 164, 164a, 167, 167a, 170, 170a, 171, 171a, 172, 172a, 173, 173a, 176, 176a, 177, 178, 179, 179a, 181, 181a, 182, 182a, 185, 185a, 186, 186a, 189, 189a, 192, 192a, 193, 193a, 194, 194a, 196, 196a, 201, 201a, 203, and 203a.

Exemplary compounds of Formula (I-a) include compounds: 126, 126a, 127, 127a, 128, and 128a.

Exemplary compounds of Formula (I-aa) include compounds: 101, 101a, 102, 102a, 103, 103a, 119, 119a, 120, and 120a.

Exemplary compounds of Formula (I-b) include compounds: 130, 130a, 131, and 131a.

Exemplary compounds of Formula (I-bb) include compounds: 111, 111a, 112, and 112a.

Exemplary compounds of Formula (I-c) include compounds: 136, and 136a.

Exemplary compounds of Formula (I-cc) include compounds: 117, 117a, 118, 118a, 135, 135a, 170, 170a, 189, and 189a.

Exemplary compounds of Formula (I-d) include compounds: 138, 138a, 140, 140a, 140b, 140c, 156, and 156a.

Exemplary compounds of Formula (I-dd) include compounds: 113, 113a, 114, 114a, 115,115a, 115b, 115c, 137,137a, 139,139a, 141,141a, 154,154a, 171,171a, 177,179,179a, 181, 181a, 201, and 201a.

Exemplary compounds of Formula (I-ee) include compounds: 167, 167a, 196, and 196a.

Exemplary compounds of Formula (I-f) include compounds: 162, and 162a.

Exemplary compounds of Formula (I-g) include compounds: 173, 173a, 192, 192a, 193, 193a, 194, and 194a.

Exemplary compounds of Formula (I-h) include compounds: 203, and 203a.

Exemplary compounds of Formula (II) include compounds: 155, 155a, 158, 158a, 163, 163a, 165, 165a, 166, 166a, 174, 174a, 175, 175a, 180, 180a, 183, 183a, 184, 184a, 187, 187a, 188, 188a, 188b, 190, 190a, 191, 191a, 195, 195a, 197, 197a, 197b, 198, 198a, 199, 199a, 200, 200a, 202, 202a, and 202b.

Exemplary compounds of Formula (II-c) include compounds: 175, 175a, 190, 190a, 191, and 191a.

Exemplary compounds of Formula (II-d) include compounds: 158, and 158a.

Exemplary compounds of Formula (II-dd) include compounds: 155, 155a, 165, and 165a.

Exemplary compounds of Formula (II-e) include compounds: 199, and 199a.

Exemplary compounds of Formula (II-ee) include compounds: 166, 166a, 197a, 197b, 202, 202a, and 202b.

Exemplary compounds of Formula (II-g) include compounds: 195, and 195a.

Exemplary compounds of Formula (II-h) include compounds: 198, 198a, 200, and 200a.

Certain examples of Formula (A) (e.g., Formula (I) or (II)) compounds were synthesized using methods involving resolution of stereoisomeric mixture(s) (e.g., SFC separation of stereoisomers). In Table C1, the resolved stereogenic centers in these compounds are labelled with an asterisk (i.e., “*”). In some instances, the stereoisomeric resolutions were performed during the last step of the synthesis, thereby providing the individual stereoisomers of the Formula (A) (e.g., Formula (I) or (II)) compounds. Alternatively, in some other instances, the resolutions were performed on an intermediate or starting material, wherein each of the constituent stereoisomers of the intermediate or starting material could be separately subjected to the subsequent steps of the synthesis to provide the respective Formula (A) (e.g., Formula (I) or (II)) compounds as separate stereoisomers. Methods of resolution and correlation between resolved intermediates and Formula (A) (e.g., Formula (I) or (II)) compounds are disclosed in the examples herein (e.g., in Examples 65, 66, 68, 69, 76, 77, 78, 79, 84, and 85). A person of ordinary skill in the art would understand that, under either approach for stereoisomeric resolution, stereoisomers having both (R)- and (S)-configurations at a resolved stereogenic center are provided. See Table C2, wherein Table C1 compounds whose stereoisomers contain the asterisk stereochemical notations are provided in non-stereogenic form, followed by the respective stereoisomers having the (R)- and (S)-configurations.

TABLE C2

| Compound | |
|---|---|
| No. | Structure |
| 140 | 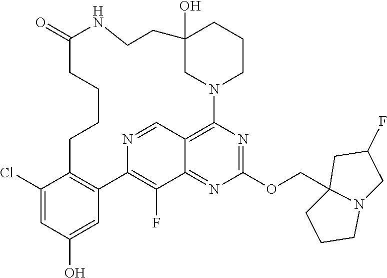 [SMILES: O=C1CCCCc2c(Cl)cc(O)cc2-c2ncc3c(nc(OCC45CCCN4CC(F)C5)nc3c2F)N2CCCC(O)(CCN1)C2] |
| | 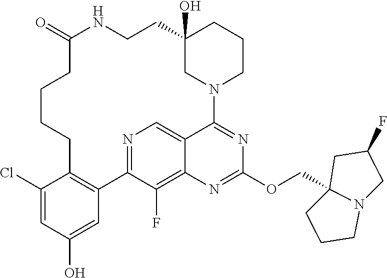 [SMILES: O=C1CCCCc2c(Cl)cc(O)cc2-c2ncc3c(nc(OC[C@@]45CCCN4C[C@H](F)C5)nc3c2F)N2CCC[C@@](O)(CCN1)C2] |
| | 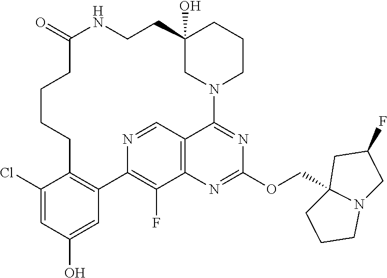 [SMILES: O=C1CCCCc2c(Cl)cc(O)cc2-c2ncc3c(nc(OC[C@@]45CCCN4C[C@H](F)C5)nc3c2F)N2CCC[C@](O)(CCN1)C2] |
| 159 | 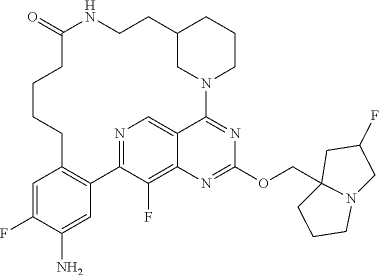 [SMILES: Nc1cc2c(cc1F)CCCCC(=O)NCCC1CCCN(C1)c1nc(OCC34CCCN3CC(F)C4)nc3c(F)c-2ncc13] |
| | 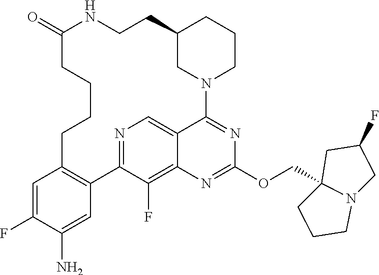 [SMILES: Nc1cc2c(cc1F)CCCCC(=O)NCC[C@H]1CCCN(C1)c1nc(OC[C@@]34CCCN3C[C@H](F)C4)nc3c(F)c-2ncc13] |
| | 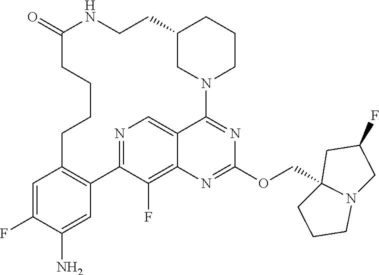 [SMILES: Nc1cc2c(cc1F)CCCCC(=O)NCC[C@@H]1CCCN(C1)c1nc(OC[C@@]34CCCN3C[C@H](F)C4)nc3c(F)c-2ncc13] |
| 188 | 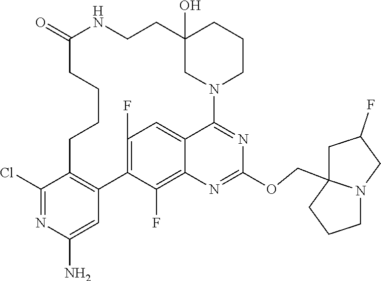 [SMILES: Nc1cc2c(c(Cl)n1)CCCCC(=O)NCCC1(O)CCCN(C1)c1nc(OCC34CCCN3CC(F)C4)nc3c(F)c-2c(F)cc13] |
| | 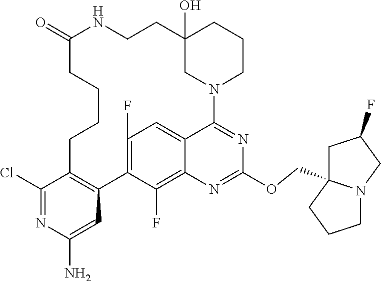 |
| | 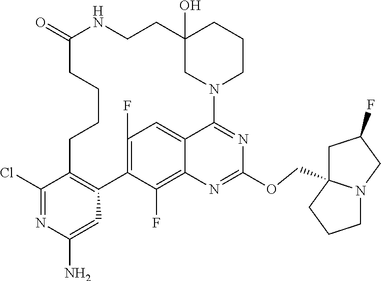 |
| 197 | 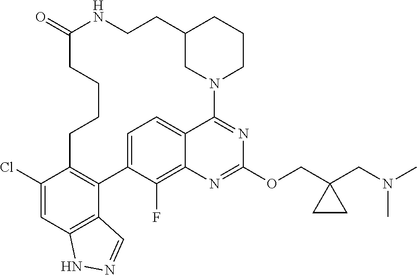 [SMILES: CN(C)CC1(COc2nc3c4ccc(c(F)c4n2)-c2c(c(Cl)cc4[nH]ncc24)CCCCC(=O)NCCC2CCCN3C2)CC1] |
| | 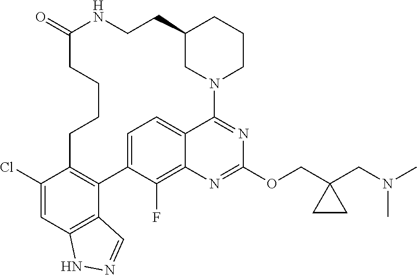 [SMILES: CN(C)CC1(COc2nc3c4ccc(c(F)c4n2)-c2c(c(Cl)cc4[nH]ncc24)CCCCC(=O)NCC[C@H]2CCCN3C2)CC1] |
| | 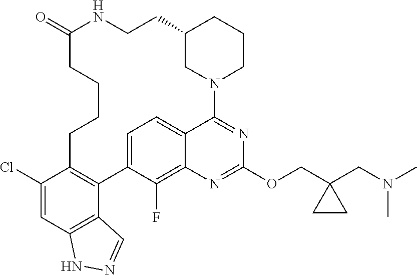 [SMILES: CN(C)CC1(COc2nc3c4ccc(c(F)c4n2)-c2c(c(Cl)cc4[nH]ncc24)CCCCC(=O)NCC[C@@H]2CCCN3C2)CC1] |
| 202 | 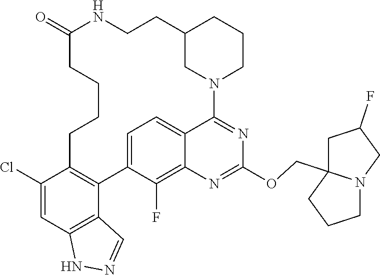 [SMILES: O=C1CCCCc2c(Cl)cc3[nH]ncc3c2-c2ccc3c(nc(OCC45CCCN4CC(F)C5)nc3c2F)N2CCCC(CCN1)C2] |
| | 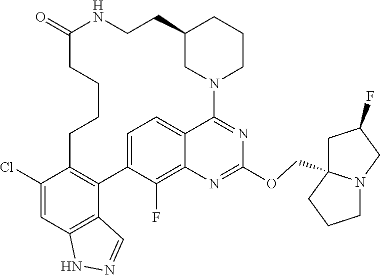 [SMILES: O=C1CCCCc2c(Cl)cc3[nH]ncc3c2-c2ccc3c(nc(OC[C@@]45CCCN4C[C@H](F)C5)nc3c2F)N2CCC[C@H](CCN1)C2] |
| | 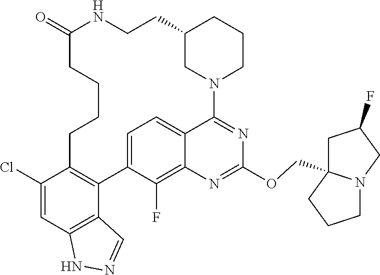 [SMILES: O=C1CCCCc2c(Cl)cc3[nH]ncc3c2-c2ccc3c(nc(OC[C@@]45CCCN4C[C@H](F)C5)nc3c2F)N2CCC[C@@H](CCN1)C2] |

Also provided herein is a KRas protein (e.g., a dysregulated KRas protein (e.g., a mutated KRas protein)) non-covalently bound with a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof. In some embodiments, Gly10 of the KRas protein interacts non-covalently with the Ring C of the compound of Formula (A) (e.g., Formula (I)), or a pharmaceutically acceptable salt thereof, (e.g., via a hydrogen bond between the carbonyl of Gly10 and an OH present on Ring C and/or via a water mediated interaction between the NH group of Gly10 and an OH present on Ring C). Without wishing to be bound by theory, in some embodiments, the interaction between Gly10 of the KRas protein and the Ring C of the compound of Formula (A) (e.g., Formula (I)), or a pharmaceutically acceptable salt thereof, facilitates the inhibition of interaction between the KRas protein and Raf-RBD. In some embodiments, Arg68 of the KRas protein interacts non-covalently with the Ring C of the compound of Formula (A) (e.g., Formula (I)), or a pharmaceutically acceptable salt thereof, (e.g., via a hydrogen bond between the guanidine of Arg68 and a CN group present on Ring C). Without wishing to be bound by theory, in some embodiments, the interaction between Arg68 of the KRas protein and the Ring C of the compound of Formula (A) (e.g., Formula (I)), or a pharmaceutically acceptable salt thereof, facilitates the inhibition of interaction between the KRas protein and Raf-RBD.

### Chemical Definitions

The term “halo” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo (e.g., —CF₃, —CHF₂, or —CH₂F).

The term “alkoxy” refers to an —O-alkyl radical (e.g., —OCH₃).

The term “alkylene” refers to a divalent alkyl (e.g., —CH₂—). Similarly, terms such as “cycloalkylene” and “heterocyclylene” refer to divalent cycloalkyl and heterocyclyl respectively. For avoidance of doubt, in “cycloalkylene” and “heterocyclylene”, the two radicals can be on the same ring carbon atom (e.g., a geminal diradical such as

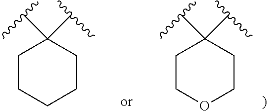 [SMILES: *C1(*)CCCCC1 ; *C1(*)CCOCC1]

or on different ring atoms (e.g., ring carbon and/or nitrogen atoms (e.g., vicinal ring carbon and/or nitrogen atoms))

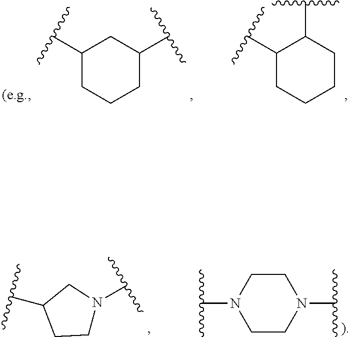 [SMILES: *C1CCCC(*)C1 ; *C1CCCCC1* ; *C1CCN(*)C1 ; *N1CCN(*)CC1]

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to mono-, bi-, tri-, or polycyclic saturated or partially unsaturated hydrocarbon groups having, e.g., 3 to 20 ring carbons, preferably 3 to 15 ring carbons, and more preferably 3 to 12 ring carbons or 3-10 ring carbons or 3-6 ring carbons, wherein the cycloalkyl group may be optionally substituted. The term “saturated” as used in this context means only single bonds present between constituent carbon atoms. Examples of saturated cycloalkyl groups include, without limitation, cyclopropyl, cyclobutyl, cyclopentyl, cyclohexyl, cycloheptyl, and cyclooctyl. Partially unsaturated cycloalkyl may have any degree of unsaturation provided that one or more double bonds is present in the cycloalkyl, none of the rings in the ring system are aromatic, and the partially unsaturated cycloalkyl group is not fully saturated overall. Examples of partially unsaturated cycloalkyl include, without limitation, cyclopentenyl, cyclohexenyl, cycloheptenyl, and cyclooctenyl. Cycloalkyl may include multiple fused and/or bridged rings. Non-limiting examples of fused/bridged cycloalkyl includes: bicyclo[1.1.0]butyl, bicyclo[2.1.0]pentyl, bicyclo[1.1.1]pentyl, bicyclo[3.1.0]hexyl, bicyclo[2.1.1]hexyl, bicyclo[3.2.0]heptyl, bicyclo[4.1.0]heptyl, bicyclo[2.2.1]heptyl, bicyclo[3.1.1]heptyl, bicyclo[4.2.0]octyl, bicyclo[3.2.1]octyl, bicyclo[2.2.2]octyl, and the like. Cycloalkyl also includes spirocyclic rings (e.g., spirocyclic bicycle wherein two rings are connected through just one atom). Non-limiting examples of spirocyclic cycloalkyls include spiro[2.2]pentyl, spiro[2.5]octyl, spiro[3.5]nonyl, spiro[3.5]nonyl, spiro[3.5]nonyl, spiro[4.4]nonyl, spiro[2.6]nonyl, spiro[4.5]decyl, spiro[3.6]decyl, spiro[5.5]undecyl, and the like.

The term “heteroaryl”, as used herein, means a mono-, bi-, tri- or polycyclic group having 5 to 20 ring atoms, alternatively 5, 6, 9, 10, or 15 ring atoms; wherein at least one ring in the system contains one or more heteroatoms independently selected from the group consisting of N, O, S (inclusive of oxidized forms such as:

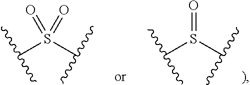

and P (inclusive of oxidized forms such as:

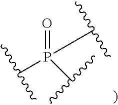 [SMILES: *P(*)(*)=O]

and at least one ring in the system is aromatic (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridinyl, pyrazolo[4,3-b]pyridinyl, tetrazolyl, chromanyl, 2,3-dihydrobenzo[b][1,4]dioxinyl, benzo[d][1,3]dioxolyl, 2,3-dihydrobenzofuranyl, tetrahydroquinolinyl, 2,3-dihydrobenzo[b][1,4]oxathiinyl, isoindolinyl, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridonyl

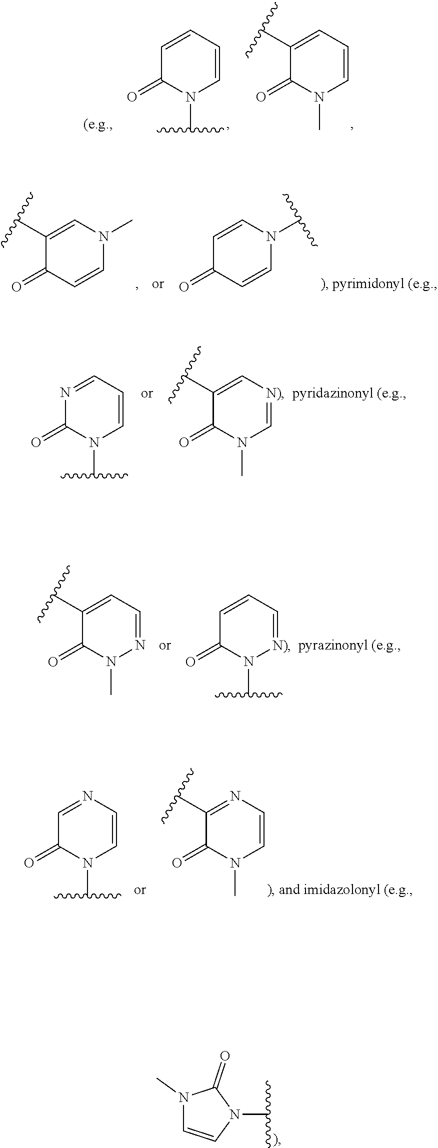

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic saturated or partially unsaturated ring system with 3-15 ring atoms (e.g., 5-8 membered monocyclic, 8-12 membered bicyclic, or 11-15 membered tricyclic ring system) having 1-3 heteroatoms if monocyclic, 1-6 heteroatoms if bicyclic, or 1-9 heteroatoms if tricyclic or polycyclic, the heteroatoms selected from O, N, S (inclusive of oxidized forms such as:

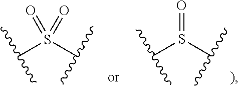

and P (inclusive of oxidized forms such as:

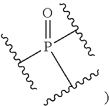 [SMILES: *P(*)(*)=O]

(e.g., carbon atoms and 1-3, 1-6, or 1-9 heteroatoms of N, O, S, or P if monocyclic, bicyclic, or tricyclic, respectively), wherein 0, 1, 2 or 3 atoms of each ring may be substituted by a substituent. The term “saturated” as used in this context means only single bonds present between constituent ring atoms and other available valences occupied by hydrogen and/or other substituents as defined herein. Examples of saturated heterocyclyl groups include piperazinyl, pyrrolidinyl, dioxanyl, morpholinyl, tetrahydrofuranyl, and the like. Partially unsaturated heterocyclyl groups may have any degree of unsaturation provided that one or more double bonds is present in the heterocyclyl, none of the rings in the ring system are aromatic, and the partially unsaturated heterocyclyl group is not fully saturated overall. Examples of partially unsaturated heterocyclyl groups include, without limitation, tetrahydropyridyl, dihydropyrazinyl, dihydropyridyl, dihydropyrrolyl, dihydrofuranyl, dihydrothiophenyl.

Heterocyclyl may include multiple fused and bridged rings. Non-limiting examples of fused/bridged heteorocyclyl includes: 2-azabicyclo[1.1.0]butyl, 2-azabicyclo[2.1.0]pentyl, 2-azabicyclo[1.1.1]pentyl, 3-azabicyclo[3.1.0]hexyl, 5-azabicyclo[2.1.1]hexyl, 3-azabicyclo[3.2.0]heptyl, octahydrocyclopenta[c]pyrrole, 3-azabicyclo[4.1.0]heptyl, 7-azabicyclo[2.2.1]heptyl, 6-azabicyclo[3.1.1]heptyl, 7-azabicyclo[4.2.0]octyl, 2-azabicyclo[2.2.2]octyl, 3-azabicyclo[3.2.1]octyl, 2-oxabicyclo[1.1.0]butyl, 2-oxabicyclo[2.1.0]pentyl, 2-oxabicyclo[1.1.1]pentyl, 3-oxabicyclo[3.1.0]hexyl, 5-oxabicyclo[2.1.1]hexyl, 3-oxabicyclo[3.2.0]heptyl, 3-oxabicyclo[4.1.0]heptyl, 7-oxabicyclo[2.2.1]heptyl, 6-oxabicyclo[3.1.1]heptyl, 7-oxabicyclo[4.2.0]octyl, 2-oxabicyclo[2.2.2]octyl, 3-oxabicyclo[3.2.1]octyl, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentyl, 4-azaspiro[2.5]octyl, 1-azaspiro[3.5]nonyl, 2-azaspiro[3.5]nonyl, 7-azaspiro[3.5]nonyl, 2-azaspiro[4.4]nonyl, 6-azaspiro[2.6]nonyl, 1,7-diazaspiro[4.5]decyl, 7-azaspiro[4.5]decyl 2,5-diazaspiro[3.6]decyl, 3-azaspiro[5.5]undecyl, 2-oxaspiro[2.2]pentyl, 4-oxaspiro[2.5]octyl, 1-oxaspiro[3.5]nonyl, 2-oxaspiro[3.5]nonyl, 7-oxaspiro[3.5]nonyl, 2-oxaspiro[4.4]nonyl, 6-oxaspiro[2.6]nonyl, 1,7-dioxaspiro[4.5]decyl, 2,5-dioxaspiro[3.6]decyl, 1-oxaspiro[5.5]undecyl, 3-oxaspiro[5.5]undecyl, 3-oxa-9-azaspiro[5.5]undecyl and the like.

As used herein, when a ring is described as being “partially unsaturated”, it means the ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

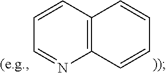

(ii) a single ring atom (spiro-fused ring systems)

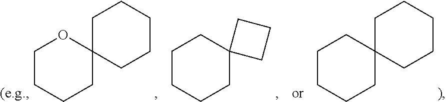

or (iii) a contiguous array of ring atoms (bridged ring systems having all bridge lengths >0)

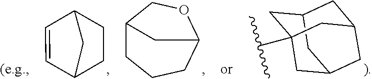

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

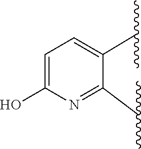 [SMILES: *c1ccc(O)nc1*]

encompasses the tautomeric form containing the moiety:

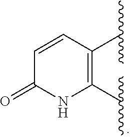 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Unless otherwise indicated, when a disclosed compound is named or depicted by a structure without specifying the stereochemistry and has one or more chiral centers, it is understood to represent all possible stereoisomers of the compound.

### Methods of Treatment

### Indications

Provided herein are methods for inhibiting a KRas protein. For example, provided herein are inhibitors of a KRas protein (e.g., a dysregulated KRas protein (e.g., a mutant KRas protein (e.g., a KRas G12D mutant protein, a KRas G12R mutant protein, or a KRas G12V mutant protein))) useful for treating or preventing diseases or disorders associated with the KRas dysregulation (i.e., a KRas-associated disease or disorder), such as a cardiovascular disease, an inflammatory and/or autoimmune disease, or a cancer (e.g., a KRas-associated cancer).

The term “KRas-associated disease or disorder” as used herein refers to diseases or disorders associated with or having a dysregulation of a KRAS gene, a KRas protein, or the expression or activity or level of any (e.g., one or more) of the same (e.g., any of the types of dysregulations of a KRAS gene, a KRas protein, or the expression or activity or level of any of the same described herein). Non-limiting examples of a KRas-associated disease or disorder include, for example, cancer, a cardiovascular disease (e.g., arteriovenous malformations), endometriosis, and an inflammatory and/or autoimmune disease (e.g., a nonmalignant syndrome of autoimmunity and abnormal leukocyte homeostasis). See, e.g., Adashek et al. Genome Med. 2020; 12: 16; Niemela et al. Blood. 2011; 117(10):2883-6; Nosan et al. Croat Med J. 2013; 54(6): 574-578; and Messina et al. Small GTPases 11.5 (2020): 312-319.

The term “mutant KRas-associated disease or disorder” as used herein refers to diseases or disorders associated with or having a KRas mutation (e.g., a KRAS gene having a mutation corresponding to a mutation in a KRas protein and/or a KRas protein having a mutation). Non-limiting examples of a mutant KRas-associated disease or disorder include, for example, cancer, a cardiovascular disease (e.g., arteriovenous malformations), endometriosis, and an inflammatory and/or autoimmune disease (e.g., a nonmalignant syndrome of autoimmunity and abnormal leukocyte homeostasis). See, e.g., Adashek et al. Genome Med. 2020; 12: 16; Niemela et al. Blood. 2011; 117(10):2883-6; Nosan et al. Croat Med J. 2013; 54(6): 574-578; and Messina et al. Small GTPases 11.5 (2020): 312-319.

The phrase “dysregulation of a KRAS gene, a KRas protein, or the expression or activity or level of any of the same” refers to a genetic mutation (e.g., a mutation in a KRAS gene that results in the expression of a KRas protein that includes a deletion of at least one amino acid as compared to a wild type KRas protein, a mutation in a KRAS gene that results in the expression of a KRas protein with one or more point mutations as compared to a wild type KRas protein, a mutation in a KRAS gene that results in the expression of a KRas protein with at least one inserted amino acid as compared to a wild type KRas protein, a gene duplication that results in an increased level of KRas protein in a cell, or a mutation in a regulatory sequence (e.g., a promoter and/or enhancer) that results in an increased level of KRas protein in a cell); an alternative spliced version of a KRas mRNA that results in a KRas protein having a deletion of at least one amino acid in the KRas protein as compared to the wild type KRas protein; or increased expression (e.g., increased levels) of a wild type KRas protein in a mammalian cell due to aberrant cell signaling and/or dysregulated autocrine/paracrine signaling (e.g., as compared to a control non-cancerous cell). As an example, a dysregulation of a KRAS gene, a KRas protein, or expression or activity, or level of any of the same, can be a mutation in a KRAS gene that encodes a KRas protein that has low GTPase activity and/or has increased signaling activity as compared to a protein encoded by a KRAS gene that does not include the mutation. As another example, a dysregulation of a KRAS gene, a KRas protein, or expression or activity, or level of any of the same, can be a KRas amplification. In some embodiments, a KRas amplification is an amplification of the wild type KRas. In some embodiments, a KRas amplification is an amplification of a mutant KRas.

A “dysregulated KRas protein” as used herein refers to (i) a KRas protein having a mutation (e.g., a deletion of at least one amino acid as compared to a wild type KRas protein, one or more point mutations as compared to a wild type KRas protein, an insertion of at least one amino acid as compared to a wild type KRas protein); (ii) a KRas protein resulting from a gene duplication event, e.g., of the gene encoding the KRas protein (e.g., the wild type KRas protein), thus resulting in an increased level and/or activity of the KRas protein (e.g., the wild type KRas protein) in a cell; (iii) a KRas protein resulting from a mutation in a regulatory sequence (e.g., a promoter and/or enhancer) that can also result in an increased level and/or activity of the KRas protein (e.g., the wild type KRas protein) in a cell); (iv) a KRas protein resulting from an alternative spliced version of a KRas mRNA that results in a KRas protein having a deletion of at least one amino acid in the KRas protein as compared to the wild type KRas protein); or (v) a KRas protein resulting from increased expression (e.g., increased levels) of a wild type KRas protein in a mammalian cell due to aberrant cell signaling and/or dysregulated autocrine/paracrine signaling (e.g., as compared to a control non-cancerous cell). In some embodiments, a dysregulated KRas protein is a dysregulated human KRas protein.

A “mutant KRas protein” as used herein refers to a KRas protein including a substitution, an insertion, a deletion, a truncation and/or a fusion relative to the wild type human KRas sequence shown in SEQ ID NO:1. For example, a mutant human KRas protein includes a substitution at any amino acid position (relative to SEQ ID NO: 1).

A “KRas G12X mutant protein” as used herein refers to a KRas protein including substitution of a glycine to any other amino acid at the twelfth amino acid position (relative to SEQ ID NO: 1).

A “KRas G12A mutant protein” as used herein refers to a KRas protein including a glycine to alanine substitution at the twelfth amino acid position (relative to SEQ ID NO: 1).

A “KRas G12C mutant protein” as used herein refers to a KRas protein including a glycine to cysteine substitution at the twelfth amino acid position (relative to SEQ ID NO: 1).

A “KRas G12D mutant protein” as used herein refers to a KRas protein including a glycine to aspartic acid substitution at the twelfth amino acid position (relative to SEQ ID NO: 1).

A “KRas G12R mutant protein” as used herein refers to a KRas protein including a glycine to arginine substitution at the twelfth amino acid position (relative to SEQ ID NO: 1).

A “KRas G12S mutant protein” as used herein refers to a KRas protein including a glycine to serine substitution at the twelfth amino acid position (relative to SEQ ID NO: 1).

A “KRas G12V mutant protein” as used herein refers to a KRas protein including a glycine to valine substitution at the twelfth amino acid position (relative to SEQ ID NO: 1).

A “KRas G13X mutant protein” as used herein refers to a KRas protein including substitution of a glycine to any other amino acid at the thirteenth amino acid position (relative to SEQ ID NO: 1).

A “KRas G13C mutant protein” as used herein refers to a KRas protein including a glycine to cysteine substitution at the thirteenth amino acid position (relative to SEQ ID NO: 1).

A “KRas G13D mutant protein” as used herein refers to a KRas protein including a glycine to aspartic acid substitution at the thirteenth amino acid position (relative to SEQ ID NO: 1).

A “KRas G13V mutant protein” as used herein refers to a KRas protein including a glycine to valine substitution at the thirteenth amino acid position (relative to SEQ ID NO: 1).

A “KRas Q61X mutant protein” as used herein refers to a KRas protein including substitution of a glutamine to any other amino acid at the sixty-first amino acid position (relative to SEQ ID NO: 1).

A “KRas Q61E mutant protein” as used herein refers to a KRas protein including a glutamine to glutamic acid substitution at the sixty-first amino acid position (relative to SEQ ID NO: 1).

A “KRas Q61H mutant protein” as used herein refers to a KRas protein including a glutamine to histidine substitution at the sixty-first amino acid position (relative to SEQ ID NO: 1).

A “KRas Q61K mutant protein” as used herein refers to a KRas protein including a glutamine to lysine substitution at the sixty-first amino acid position (relative to SEQ ID NO: 1).

A “KRas Q61L mutant protein” as used herein refers to a KRas protein including a glutamine to leucine substitution at the sixty-first amino acid position (relative to SEQ ID NO: 1).

A “KRas Q61P mutant protein” as used herein refers to a KRas protein including a glutamine to proline substitution at the sixty-first amino acid position (relative to SEQ ID NO: 1).

A “KRas Q61R mutant protein” as used herein refers to a KRas protein including a glutamine to arginine substitution at the sixty-first amino acid position (relative to SEQ ID NO: 1).

A “KRas inhibitor” as used herein includes any compound exhibiting KRas protein inactivation activity (e.g., inhibiting or decreasing KRas signaling activity). A KRas inhibitor as described herein has an IC₅₀ value of 1 μM or less in a nucleotide exchange assay as described herein, an IC₅₀ value of 1 μM or less in a Raf kinase interaction assay as described herein, or both. In some embodiments, a KRas inhibitor inhibits the signaling activity of a wild type KRas protein. In some embodiments, a KRas inhibitor inhibits the signaling activity of a dysregulated KRas protein, for example, resulting in a decrease in activated Raf or other downstream effectors, such as ERK. In some embodiments, a KRas inhibitor inhibits the signaling activity of a mutant KRas protein. In some embodiments, a KRas inhibitor inhibits both the signaling activity of a wild-type KRas protein and the signaling activity of one or more mutant KRas proteins and can be termed a “pan KRas inhibitor”. In some embodiments, a KRas inhibitor inhibits one or more mutant KRas proteins, and such a KRas inhibitor can be termed a “mutant KRas inhibitor”, and also termed by the mutant(s) it inhibits. For example, a KRas inhibitor that inhibits KRas G12R mutant protein could be termed a “KRas G12R inhibitor”. As another example, a KRas inhibitor that inhibits both KRas G12C mutant protein and KRas G12D mutant protein could be termed a “KRas G12C inhibitor” and/or a “KRas G12D inhibitor”. In some embodiments, a “mutant KRas inhibitor” inhibits two or more mutant KRas proteins and can be termed a “pan mutant KRas inhibitor”. In some embodiments, a pan mutant KRas inhibitor inhibits two or more mutant KRas proteins selected from the group consisting of: a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas G13D mutant protein, a KRas G13V mutant protein, a KRas Q61E mutant protein, a KRas Q61H mutant protein, a KRas Q61K mutant protein, a KRas Q61L mutant protein, a KRas Q61P mutant protein, and a KRas Q61R mutant protein. For example, a “KRas G12X inhibitor” can inhibit two or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. As yet another example, a KRas inhibitor that inhibits a KRas G13D mutant protein could be termed a “KRas G13D inhibitor”. In some embodiments, a KRas inhibitor can inhibit a KRas protein having one or more mutations, and such a KRas inhibitor can be termed a “mutant KRas inhibitor” whether or not the mutant KRas inhibitor also inhibits wild type KRas protein. In some embodiments, a KRas inhibitor is a mutant KRas inhibitor. In some embodiments, a KRas inhibitor is an allosteric inhibitor.

Compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are KRas inhibitors. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a mutant KRas inhibitor. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas G13D mutant protein, a KRas G13V mutant protein, a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12D mutant protein, a KRas G12R mutant protein, a KRas G12V mutant protein, or a combination thereof. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12D mutant protein, a KRas G12V mutant protein, or both. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12R mutant protein, a KRas G12V mutant protein, or both. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12D mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12V mutant protein.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12X inhibitor. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits four or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits five or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits four or more mutant KRas proteins selected from the group consisting of: a KRas G12A mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12A mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12A mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12C mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, does not inhibit a KRas G12C mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12D mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, does not inhibit a KRas G12D mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12S mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12V mutant protein.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (TT-f), (II-g), or (II-h)), or Formula (Aa)), or a pharmaceutically acceptable salt thereof, is a KRas G13X inhibitor. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G13C mutant protein, a KRas G13D mutant protein, and a KRas G13V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G13C mutant protein, a KRas G13D mutant protein, and a KRas G13V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G13C mutant protein, a KRas G13D mutant protein, and a KRas G13V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G13C mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G13D mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G13V mutant protein.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas Q61X inhibitor. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (TT-f), (II-g), or (II-h)), or Formula (Aa)), or a pharmaceutically acceptable salt thereof, inhibits one or more mutant KRas proteins selected from the group consisting of: a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas Q61E mutant protein, a KRas Q61H mutant protein, a KRas Q61K mutant protein, a KRas Q61L mutant protein, a KRas Q61P mutant protein, and a KRas mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (TT-f), (II-g), or (II-h)), or Formula (Aa)), or a pharmaceutically acceptable salt thereof, inhibits three or more mutant KRas proteins selected from the group consisting of: a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits four or more mutant KRas proteins selected from the group consisting of: a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits five or more mutant KRas proteins selected from the group consisting of: a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas Q61E mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas Q61H mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas Q61K mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas Q61L mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas Q61P mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas Q61R mutant protein.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12X mutant protein, a KRas G13X mutant protein, and a KRas Q61X mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (TT-f), (II-g), or (II-h)), or Formula (Aa)), or a pharmaceutically acceptable salt thereof, inhibits two or more mutant KRas proteins selected from the group consisting of: a KRas G12X mutant protein, a KRas G13X mutant protein, and a KRas Q61X mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant human KRas proteins selected from the group consisting of: a KRas G12X mutant protein, a KRas G13X mutant protein, and a KRas Q61X mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12X mutant protein, a KRas G13X mutant protein, and a KRas Q61X mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas G13D mutant protein, a KRas G13V mutant protein, a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas G13D mutant protein, a KRas G13V mutant protein, a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas G13D mutant protein, a KRas G13V mutant protein, a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits four or more mutant KRas proteins selected from the group consisting of: a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas G13D mutant protein, a KRas G13V mutant protein, a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, five or more mutant KRas proteins selected from the group consisting of: a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas G13D mutant protein, a KRas G13V mutant protein, a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas G13D mutant protein, a KRas G13V mutant protein, a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12A mutant protein, a KRas G12D mutant protein, a KRas G12R mutant protein, a KRas G12S mutant protein, a KRas G12V mutant protein, a KRas G13C mutant protein, a KRas G13D mutant protein, a KRas G13V mutant protein, a KRas Q61E mutant protein, a KRas Q61H mutant protein, a KRas Q61K mutant protein, a KRas Q61L mutant protein, a KRas Q61P mutant protein, and a KRas Q61R mutant protein.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12C mutant protein, a KRas G12D mutant protein, a KRas G12R mutant protein, a KRas G12V mutant protein, a KRas G13D mutant protein, and a KRas Q61H mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12C mutant protein, a KRas G12D mutant protein, a KRas G12R mutant protein, a KRas G12V mutant protein, a KRas G13D mutant protein, and a KRas Q61H mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12C mutant protein, a KRas G12D mutant protein, a KRas G12R mutant protein, a KRas G12V mutant protein, a KRas G13D mutant protein, and a KRas Q61H mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12C mutant protein, a KRas G12D mutant protein, a KRas G12R mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12C mutant protein, a KRas G12D mutant protein, a KRas G12R mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12C mutant protein, a KRas G12D mutant protein, a KRas G12R mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12D mutant protein, a KRas G12R mutant protein, and a KRas G12V mutant protein. In some embodiments, compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12D mutant protein, a KRas G12R mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12D mutant protein, a KRas G12R mutant protein, and a KRas G12V mutant protein. In some such embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are useful for treating a bladder cancer.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12C mutant protein, a KRas G12D mutant protein, a KRas G12V mutant protein, and a KRas G13D mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (TT-f), (II-g), or (II-h)), or Formula (Aa)), or a pharmaceutically acceptable salt thereof, inhibits two or more mutant KRas proteins selected from the group consisting of: a KRas G12C mutant protein, a KRas G12D mutant protein, a KRas G12V mutant protein, and a KRas G13D mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12C mutant protein, a KRas G12D mutant protein, a KRas G12V mutant protein, and a KRas G13D mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12C mutant protein, a KRas G12D mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12C mutant protein, a KRas G12D mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12C mutant protein, a KRas G12D mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12D mutant protein, a KRas G12V mutant protein, or both. In some such embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are useful for treating a cervical cancer.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (TT-f), (II-g), or (II-h)), or Formula (Aa)), or a pharmaceutically acceptable salt thereof, inhibits one or more mutant KRas proteins selected from the group consisting of: a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas G13D mutant protein, a KRas G13V mutant protein, a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12D mutant protein, a KRas G12R mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12D mutant protein, a KRas G12R mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12D mutant protein, a KRas G12R mutant protein, and a KRas G12V mutant protein. In some such embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are useful for treating a colorectal cancer.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12C mutant protein, a KRas G12D mutant protein, a KRas G12S mutant protein, a KRas G12V mutant protein, a KRas G13C mutant protein, a KRas G13D mutant protein, a KRas G13V mutant protein, a KRas Q61H mutant protein, and a KRas Q61L mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12C mutant protein, a KRas G12D mutant protein, a KRas G12S mutant protein, a KRas G12V mutant protein, a KRas G13C mutant protein, a KRas G13D mutant protein, a KRas G13V mutant protein, a KRas Q61H mutant protein, and a KRas Q61L mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12C mutant protein, a KRas G12D mutant protein, a KRas G12S mutant protein, a KRas G12V mutant protein, a KRas G13C mutant protein, a KRas G13D mutant protein, a KRas G13V mutant protein, a KRas Q61H mutant protein, and a KRas Q61L mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12C mutant protein, a KRas G12D mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12C mutant protein, a KRas G12D mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12C mutant protein, a KRas G12D mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12D mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12D mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12A mutant protein, a KRas G12D mutant protein, and a KRas G12V mutant protein. In some such embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are useful for treating an endometrial cancer.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12C mutant protein, a KRas G12D mutant protein, a KRas G12S mutant protein, a KRas G12V mutant protein, a KRas G13C mutant protein, a KRas G13D mutant protein, and a KRas Q61H mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12C mutant protein, a KRas G12D mutant protein, a KRas G12S mutant protein, a KRas G12V mutant protein, a KRas G13C mutant protein, a KRas G13D mutant protein, and a KRas Q61H mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12C mutant protein, a KRas G12D mutant protein, a KRas G12S mutant protein, a KRas G12V mutant protein, a KRas G13C mutant protein, a KRas G13D mutant protein, and a KRas Q61H mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12C mutant protein, a KRas G12D mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12C mutant protein, a KRas G12D mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12C mutant protein, a KRas G12D mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12D mutant protein, a KRas G12V mutant protein, or both. In some such embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are useful for treating an esophageal or stomach cancer.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas G13D mutant protein, a KRas G13V mutant protein, a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas G13D mutant protein, a KRas G13V mutant protein, a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas G13D mutant protein, a KRas G13V mutant protein, a KRas Q61E mutant protein, a KRas Q61H mutant protein, a KRas Q61K mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12D mutant protein, a KRas G12R mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12D mutant protein, a KRas G12R mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12D mutant protein, a KRas G12R mutant protein, and a KRas G12V mutant protein. In some such embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are useful for treating a leukemia.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (TT-f), (II-g), or (II-h)), or Formula (Aa)), or a pharmaceutically acceptable salt thereof, inhibits one or more mutant KRas proteins selected from the group consisting of: a KRas G12C mutant protein, a KRas G12D mutant protein, a KRas G12R mutant protein, a KRas G13D mutant protein, a KRas G13V mutant protein, a KRas a KRas Q61K mutant protein, a KRas Q61L mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12C mutant protein, a KRas G12D mutant protein, a KRas G12R mutant protein, a KRas G13D mutant protein, a KRas G13V mutant protein, a KRas a KRas Q61K mutant protein, a KRas Q61L mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12C mutant protein, a KRas G12D mutant protein, a KRas G12R mutant protein, a KRas G13D mutant protein, a KRas G13V mutant protein, a KRas a KRas Q61K mutant protein, a KRas Q61L mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12C mutant protein, a KRas G12D mutant protein, and a KRas G12R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12C mutant protein, a KRas G12D mutant protein, and a KRas G12R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12C mutant protein, a KRas G12D mutant protein, and a KRas G12R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12D mutant protein, and a KRas G12R mutant protein, or both. In some such embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are useful for treating a melanoma.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12C mutant protein, a KRas G12D mutant protein, a KRas G12S mutant protein, a KRas G12V mutant protein, a KRas G13C mutant protein, a KRas G13D mutant protein, a KRas Q61H mutant protein, and a KRas Q61L mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12C mutant protein, a KRas G12D mutant protein, a KRas G12S mutant protein, a KRas G12V mutant protein, a KRas G13C mutant protein, a KRas G13D mutant protein, a KRas Q61H mutant protein, and a KRas Q61L mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12C mutant protein, a KRas G12D mutant protein, a KRas G12S mutant protein, a KRas G12V mutant protein, a KRas G13C mutant protein, a KRas G13D mutant protein, a KRas Q61H mutant protein, and a KRas Q61L mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12C mutant protein, a KRas G12D mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12C mutant protein, a KRas G12D mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12C mutant protein, a KRas G12D mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12D mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12D mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12A mutant protein, a KRas G12D mutant protein, and a KRas G12V mutant protein. In some such embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are useful for treating a lung cancer (e.g., non-small cell lung cancer).

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas Q61H mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas Q61H mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12C mutant protein, a KRas G12D mutant protein, a KRas G12R mutant protein, a KRas G12S mutant protein, a KRas G12V mutant protein, a KRas G13C mutant protein, a KRas Q61H mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12C mutant protein, a KRas G12D mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12D mutant protein, a KRas G12R mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12D mutant protein, a KRas G12R mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12D mutant protein, a KRas G12R mutant protein, and a KRas G12V mutant protein. In some such embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are useful for treating a pancreatic cancer.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12R mutant protein, a KRas G12S mutant protein, a KRas G12V mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12R mutant protein, a KRas G12S mutant protein, a KRas G12V mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12R mutant protein, a KRas G12S mutant protein, a KRas G12V mutant protein, a KRas Q61L mutant protein, a KRas Q61P mutant protein, and a KRas Q61R mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits three or more mutant KRas proteins selected from the group consisting of: a KRas G12A mutant protein, a KRas G12R mutant protein, a KRas G12S mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits one or more mutant KRas proteins selected from the group consisting of: a KRas G12A mutant protein, a KRas G12R mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits two or more mutant KRas proteins selected from the group consisting of: a KRas G12A mutant protein, a KRas G12R mutant protein, and a KRas G12V mutant protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits a KRas G12A mutant protein, a KRas G12R mutant protein, and a KRas G12V mutant protein. In some such embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are useful for treating a testicular cancer (e.g., seminoma).

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (TT-f), (II-g), or (II-h)), or Formula (Aa)), or a pharmaceutically acceptable salt thereof, can bind to a KRas protein in the GTP-bound state. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can bind selectively to a KRas protein in the GTP-bound state.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (TT-f), (II-g), or (II-h)), or Formula (Aa)), or a pharmaceutically acceptable salt thereof, can bind to a KRas protein in the GDP-bound state. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can bind selectively to a KRas protein in the GDP-bound state.

An exemplary sequence of mature human KRas protein is shown below (UniProtKB entry P01116) (SEQ ID NO: 1)

MTEYKLVVVG AGGVGKSALT IQLIQNHFVD EYDPTIEDSY
RKQVVIDGET CLLDILDTAG QEEYSAMRDQ YMRTGEGFLC
VFAINNTKSF EDIHHYREQI KRVKDSEDVP MVLVGNKCDL
PSRTVDTKQA QDLARSYGIP FIETSAKTRQ RVEDAFYTLV
REIRQYRLKK ISKEEKTPGC VKIKKCIIM

As used herein, “selective” or “selectively”, when referring to an assayed compound, indicates at least a 5-fold (e.g., at least a 10-fold, at least a 25-fold, at least a 50-fold, or at least a 100-fold) superior performance in an assay (e.g., binding affinity and/or potency) for a specified condition with reference to a comparator protein variant in the assay. For example, if a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, binds “selectively” to a KRas G12X mutant protein over the wild type KRas protein as determined by a surface plasmon resonance (SPR) assay, then the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, has at least a 5-fold (e.g., at least a 10-fold, at least a 25-fold, at least a 50-fold, or at least a 100-fold) smaller K_{D} value for any one or more KRas mutant proteins selected from the group consisting of the KRas G12X mutant proteins than for the wild type KRas protein when measured by the SPR assay. As a further example, if a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, “selectively” reduces the viability the KRas G12V mutant protein-expressing cells over the cells expressing KRas G12C protein as determined by a cell proliferation assay, then the compound has at least a 5-fold (e.g., at least a 10-fold, at least a 25-fold, at least a 50-fold, or at least a 100-fold) EC₅₀ value for the KRas G12V mutant protein-expressing cells than for the KRas G12C protein-expressing cells when measured by the cell proliferation assay. In another example, if a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, “selectively” inhibits a KRas G13X mutant protein over the wild type KRas protein as determined by a Raf kinase interaction assay, then the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, has at least a 5-fold (e.g., at least a 10-fold, at least a 25-fold, at least a 50-fold, or at least a 100-fold) smaller IC₅₀ value for the KRas G13X protein than for the wild type KRas protein when measured by the Raf kinase interaction assay. As a further example, if a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, “selectively” inhibits the KRas G12R mutant protein over the wild type KRas protein as determined by a nucleotide exchange assay, then the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, has at least a 5-fold (e.g., at least a 10-fold, at least a 25-fold, at least a 50-fold, or at least a 100-fold) smaller IC₅₀ value for the KRas G12R mutant protein than for the wild type KRas protein when measured by the nucleotide exchange assay.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a pan mutant KRas inhibitor (i.e., can inhibit two or more mutant KRas proteins (e.g., two or more of a KRas G12A mutant protein, a KRas G12D mutant protein, a KRas G12R mutant protein, and a KRas G12V mutant protein)). For example, such a compound can inhibit each mutant KRas protein (e.g., two or more mutant KRas proteins) with an IC₅₀ of less than 1 μM (e.g., less than 750 nM, less than 500 nM, or less than 200 nM). As another example, such a compound can inhibit ERK phosphorylation in cell lines each expressing a mutant KRas protein with an independent IC₅₀ of less than 1 μM (e.g., less than 750 nM, less than 500 nM, or less than 200 nM) in at least of the two cell lines. For example, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can inhibit ERK phosphorylation in a cell line expressing a KRas G12R mutant protein with an IC₅₀ of less than 1 μM, and the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can inhibit ERK phosphorylation in a cell line expressing a KRas G12V mutant protein with an IC₅₀ of less than 1 μM. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a pan KRas inhibitor (i.e., the compound can inhibit wild type KRas and one or more mutant KRas proteins). In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, does not inhibit certain KRas proteins (e.g., wild type KRas or one or more dysregulated KRas proteins). For example, such a compound can inhibit the interaction between a KRas protein it does not inhibit (e.g., a dysregulated KRas protein) and one or more Raf proteins with an IC₅₀ of 1 μM or greater than 1 μM (e.g., greater than 2 μM, greater than 5 μM, greater than 10 μM, or greater than 30 μM). As another example, such a compound can inhibit ERK phosphorylation in cell lines expressing the KRas protein it does not inhibit (e.g., a dysregulated KRas protein) with an IC₅₀ of 1 μM or greater than 1 μM (e.g., greater than 2 μM, greater than 5 μM, greater than 10 μM, or greater than 30 μM).

The ability of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, to bind to a KRas protein can be measured, for example, by a direct determination method (e.g., surface plasmon resonance or isothermal titration calorimetry); by radio labelling the compound prior to binding, isolating the compound/protein complex, and determining the amount of radio label bound; or by running a competition experiment where new compounds are incubated with the protein bound to known radioligands. As another example, the occupancy of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can be determined using a proximity-based technique, such as time-resolved Fluorescence Resonance Energy Transfer (FRET); for instance, using a labeled probe that binds mutually exclusively with the inhibitor, and using an antibody that binds to a position on the protein separate from where the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, binds (for example, an antibody that binds to an N-terminal tag). It will be understood that the antibody and probe can be tagged with any appropriate FRET pair. See, e.g., International Publication Nos. WO 2021/041671, WO 2021/120890, and U.S. Publication No. US 2021/0179633.

In some cases, binding affinities (e.g., as measured by dissociation constant K_{D}) of the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof with a KRas protein (e.g., a wild type KRas protein or a mutant KRas protein) in the GDP-bound and/or GTP-bound state can be measured using methods known in the art (e.g., using SPR (e.g., using one or more methods described herein (e.g., using the methods described in Example B1 herein))). Binding affinity with the KRas protein in the GDP-bound state can be measured by loading the KRas protein with GDP (e.g., at the concentrations described in Example B1). Binding affinity with the KRas protein in the GTP-bound state can be measured by loading the KRas protein with GMPPNP (e.g., at the concentrations described in Example B1).

Another exemplary assay for determining the potency of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, includes measuring the effect of the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, on cell proliferation. Cell proliferation assays can be performed in a number of formats, including 2D and 3D. Similarly, a cell proliferation assay can be performed with any appropriate cell line, including, for example, PSN1, HUPT3, KP2, CAL62, TCCPAN2, PK8, PATC50, H358, MIAPaCa-2, CALU1, AGS, A427, ASPC1, HKA1, KMS20, A549, LS123, HTK, H727, H441, HCT116, H460, A375, NCI-H1993, PC9, MKN1, NCI-H211, NCI-H424, and/or NCI-H526. In some embodiments, the cell line can be AGS, A427, ASPC1, H727, and/or H441. As an illustrative example, a 3D cell proliferation assay can include growing cells in a 3D medium, contacting the cells with a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, measuring the cellular proliferation using an appropriate reagent (e.g., CELLTITERGLO® 3D), and then comparing the signal from the experiment with the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, to the signal from a control experiment (e.g., lacking a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa))). As another illustrative example, a 2D cell proliferation assay can include plating cells onto a growth surface, optionally letting the cells grow for a period of time, contacting the cells with a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, measuring the cellular proliferation using an appropriate reagent (e.g., CELLTITERGLO®), and then comparing the signal from the experiment with a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, to the signal from a control experiment (e.g., lacking a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof). In some embodiments, cellular proliferation can be assessed using a platform for live cell imaging (e.g., an INCUCYTE® SX5 Live-Cell Analysis Instrument). See also, e.g., U.S. Publication No. 2021/0179633, 2021/0230142, and 2019/0284144.

As another example, the potency and/or efficacy of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can be evaluated in an animal model, for example, a xenograft model (e.g., using an established cancer cell line such as H727, H441, AGS, A427 and/or ASPC1 or a patient-derived xenograft (PDX) model). See, e.g., U.S. Publication No. 2021/0179633.

Additional assays can include, for example, assays based on hydrogen exchange (HX) mass spectrometry. Such assays can be useful, for example, to evaluate whether a compound (e.g., a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof) stabilizes the GTP-bound state or GDP-bound state of a KRas protein (e.g., a dysregulated KRas protein, e.g., a mutant KRas protein (e.g., a KRas G12D mutant protein, a KRas G12R mutant protein, or a KRas G12V mutant protein)). In such assays, the rate of hydrogen exchange of the backbone amide hydrogens can be measured for a KRas protein (e.g., a dysregulated KRas protein, e.g., a mutant KRas protein (e.g., a KRas G12D mutant protein, a KRas G12R mutant protein, or a KRas G12V mutant protein)) bound to a non-hydrolyzable GTP mimic (GMPPNP), GDP, or a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof. See, e.g., Lim et al. Angew Chem Int Ed Engl. 2014; 53(1): 199-204.

In some embodiments, potency of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, as provided herein can be determined by EC₅₀ value. A compound with a lower EC₅₀ value, as determined under substantially similar conditions, is a more potent inhibitor relative to a compound with a higher EC₅₀ value. In some embodiments, an EC₅₀ value can be determined (e.g., using a KRas-dependent phosphorylation level (e.g., a phosphoERK level (sometimes called a “pERK” level)) or using a cell viability assay) in cells (e.g., in tumor cells, (e.g., cell lines such as PSN1, HUPT3, KP2, CAL62, TCCPAN2, PK8, PATC50, H358, MIAPaCa-2, CALU1 AGS, A427, ASPC1, HKA1, KMS20, A549, LS123, HTK, H727, HCT116, H460, A375, NCI-H1993, PC9, MKN1, NCI-H211, NCI-H424, and/or NCI-H526) expressing a KRas protein, such as a dysregulated KRas protein (e.g., a mutant KRas protein or an amplified KRas protein), or a fragment thereof).

In some embodiments, potency of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, as provided herein can also be determined by IC₅₀ value. A compound with a lower IC₅₀ value, as determined under substantially similar conditions, is a more potent inhibitor relative to a compound with a higher IC₅₀ value. In some embodiments, an IC₅₀ value can be determined (e.g., using a KRas-dependent phosphorylation level (e.g., a phosphoERK level) or using a cell viability assay), in cells (e.g., in tumor cells, (e.g., cell lines such as PSN1, HUPT3, KP2, CAL62, TCCPAN2, PK8, PATC50, H358, MIAPaCa-2, CALU1 AGS, A427, ASPC1, HKA1, KMS20, A549, LS123, HTK, H727, HCT116, H460, A375, NCI-H1993, PC9, MKN1, NCI-H211, NCI-H424, and/or NCI-H526) expressing a KRas protein, such as a dysregulated KRas protein (e.g., a mutant KRas protein or an amplified KRas protein), or a fragment thereof).

In some embodiments, measuring the potency of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, includes measuring the phosphorylation of a downstream kinase, such as ERK (e.g., ERK1 and/or ERK2) or MEK. Such assays can be used to measure the inhibition of KRas signaling activity, for instance, in a cell line (e.g., PSN1, HUPT3, KP2, CAL62, TCCPAN2, PK8, PATC50, H358, MIAPaCa-2, CALU1 AGS, A427, ASPC1, HKA1, KMS20, A549, LS123, HTK, H727, H441, HCT116, H460, A375, NCI-H1993, PC9, MKN1, NCI-H211, NCI-H424, and/or NCI-H526 (e.g., AGS, A427, ASPC1, H727, and/or H441)). For example, cells can be contacted with a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof for a period of time, then lysed or permeabilized, and total ERK or MEK and phosphoERK or phosphoMEK content can be determined (e.g., using antibodies, or a kit, such as Invitrogen InstantOne ERK1/ERK2 (Phospho) [pT202/pY204]/[pT185/pY187] ELISA, MesoScale Discovery p/t ERK1/2, AlphaScreen SUREFIRE® p-ERK1/2 (Thr202/Tyr204), or an HTRF® Phospho-ERK (Thr202/Tyr204) cellular kit (CisBio)). In some embodiments, multiple concentrations of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof can be used to construct a dose response curve. See, e.g., International Publication No. WO 2021/041671, U.S. Publication Nos. 2021/0122764, 2018/0334454, 2021/0179633, 2018/0334454, and 2019/0144444.

An exemplary ERK phosphorylation protocol follows. In some embodiments, an ERK phosphorylation assay can be carried out using the AlphaLisa SUREFIRE® Ultra Multiplex Phospho/Total ERK1/2 (Thr202/Tyr204) Assay Kit. In a plate (e.g., a white, opaque-bottom Perkin Elmer CulturPlate-384 (product number 6007680)), cells are seeded at the desired concentration one day prior to treatment with compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, and incubated overnight in a standard 37° C., 5% CO₂ humidified incubator. The cells can be any cells of interest, such as MIAPACA2 (KRas G12C), H358 (KRas G12C), AGS (KRas G12D), ASPC1 (KRas G12D), GP2D (KRas G12D), LS180 (KRas G12D), Panc04.03 (KRas G12D), HPAFII (KRas G12D), Panc02.03 (KRas G12D), A427 (KRas G12D), HPAC (KRas G12D), TCCPAN2 (KRas G12R), PSN1 (KRas G12R), KP2 (KRas G12R), LS123 (KRas G12S), SW620 (KRas G12V), H727 (KRas G12V), CFPAC1 (KRas G12V), CAPAN1 (KRas G12V), RKN (KRas G12V), H441 (KRas G12V), SW480 (KRas G12V), PACADD159 (KRas G12V/G12S), HS766T (KRas Q61H), H460 (KRas Q61H), PANC0213 (KRas Q61R), or A3735 (KRas WT). The day after seeding, compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are dispensed into the treatment plates (e.g., using a Tecan D300e compound printer in 9-point DRC format (1:3 dilution), 10-μM top concentration, in triplicate). Treatment plates are then returned to a standard 37° C., 5% CO₂ humidified incubator for the pre-determined treatment time. Following compound treatment, all media is removed from the treatment plate(s), and the cells are subsequently lysed (e.g., using 1× Lysis Buffer in accordance with manufacturer protocol). Next, the Acceptor Mix (prepared in accordance with manufacturer's protocol) is added to each well of the assay plate and incubated on an orbital shaker at room temperature for 2 hours. Following incubation with the Acceptor Mix, the Donor Mix (prepared in accordance with manufacturer protocol) is added to each well of the assay plate, covered to protect from light, and incubated on an orbital shaker at room temperature overnight. Assay plates are read the following day (e.g., on a BMG Labtech PHERAstar FSX microplate reader). Data are then analyzed by calculating the ratio of ERK1/2-phosphorylation relative to Total ERK1/2 for each individual well.

$$Ratio p ERK 1 / 2 = \frac{615 nm Signal ( pERK 1 / 2 )}{545 nm Signal ( Total ERK 1 / 2 )}$$

The replicate ratios for each concentration are averaged and normalized to a DMSO control or other corresponding co-treatment before performing a variable slope (4-parameter), non-linear regression curve fit for each compound of interest. Data can be reported as IC₅₀ values.

In some embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, inhibit ERK phosphorylation in a cell line expressing a KRas protein (e.g., a dysregulated KRas protein (e.g., a mutant KRas protein (e.g., a KRas G12D mutant protein, a KRas G12R mutant protein, or a KRas G12V mutant protein))) with an IC₅₀ of less than 1 μM (e.g., less than 750 nM, less than 500 nM, or less than 200 nM). In some embodiments, the compounds inhibit ERK phosphorylation in a cell line expressing the KRas protein (e.g., a dysregulated KRas protein (e.g., a mutant KRas protein (e.g., a KRas G12D mutant protein, a KRas G12R mutant protein, or a KRas G12V mutant protein))) with an IC₅₀ of less than 200 nM (e.g., less than 150 nM, less than 200 nM, less than 100 nM, less than 10 nM, less than 1 nM). For example, the compounds can inhibit ERK phosphorylation in a cell line expressing the KRas protein (e.g., a dysregulated KRas protein (e.g., a mutant KRas protein (e.g., a KRas G12D mutant protein, a KRas G12R mutant protein, or a KRas G12V mutant protein))) with an IC₅₀ of 0.1 nM to 100 nM, 0.1 nM to 50 nM, 1 nM to 50 nM, or 1 nM to 20 nM.

In some cases, a KRas A59G mutant protein (e.g., as a single mutant or as a double mutant with another mutation of interest, e.g., KRas G12X) can be used to “lock” the KRas protein in the GTP-bound state (e.g., by abrogating the GTPase activity of the protein); such an assay can be useful, for example, to determine the affinity of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof for the GTP-bound state and/or to determine the effect of the compound on downstream signaling (e.g., interaction with an RBD and/or the phosphorylation of a downstream kinase, such as ERK), potentially independent of the GTP cycling of the KRas protein. See, e.g., Hall, et al. Proceedings of the National Academy of Sciences 99.19 (2002): 12138-12142; Lu, et al. Biochemistry 57.3 (2018): 324-333; Lim, et al. bioRxiv (2021) (doi: 10.1101/2021.09.11.459913).

In some embodiments, the potency of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, as a KRas inhibitor can be evaluated by its effect on the nucleotide exchange of GDP for GTP. For example, nucleotide exchange can be measured via the increase in fluorescence of protein-bound N-methylanthraniloyl (MANT)-GDP upon the addition of an excess amount of a non-hydrolyzable GTP analog such as guanosine-5′-[(β,γ)-imido]triphosphate (GppNHp, sometimes also referred to as GMPPNP), when exchange is inhibited. See, e.g., Kanie and Jackson, Bio Protoc. 2018; 8(7): e2795. As another example, nucleotide exchange can be measured via the decrease in fluorescence of an incubated mixture of KRas protein-bound fluorophore-tagged GDP (e.g., Bodipy-GDP (e.g., EDA-GTP-DY-647P1)) and a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, followed by treatment with unlabeled GTP. In such an assay, an exchange of fluorophore-tagged GDP (e.g., Bodipy-GDP) for unlabeled GTP results in a reduced TR-FRET signal. As another example, nucleotide exchange can be measured via the increase in fluorescence of an incubated mixture of KRas protein-bound GDP and a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, followed by treatment with labeled GTP. In such an assay, an exchange of GDP for labeled GTP results in an increased FRET signal. See, e.g., International Publication No. WO 2020/085493 and U.S. Publication Nos. 2021/0122764, 2021/0269434, and 2018/0334454. In some embodiments of nucleotide exchange assays, a guanine nucleotide exchange factor (e.g., SOS1) can be added to accelerate nucleotide exchange.

Inhibition of SOS1-catalyzed exchange of GDP for GTP on the KRas protein by compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof can be measured using methods known in the art (e.g., using one or more methods described herein (e.g., using methods described in Example B2 herein)). Additional examples of in vitro assays include assays that determine inhibition of the GTPase activity of KRas protein. In some embodiments, the potency of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can be evaluated by its effect on GTPase activity (or lack thereof, as a decrease in GTPase activity is generally believed to be associated with aberrant signaling). For example, GTPase activity of a KRas protein can be measured using a phosphate assay system that continuously measures phosphate release. In some embodiments, a purine nucleoside phosphorylase-based (PNP) assay can be used to measure GTPase activity of a KRas protein. See, e.g., Hunter et al. Mol Cancer Res. 2015; 13(9):1325-35. In some embodiments, an enzyme-linked immunosorbent assay (ELISA) can be used to measure the effect of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, on the GTPase activity of a KRas protein (e.g., a dysregulated KRas protein, e.g., a mutant KRas protein (e.g., a KRas G12D mutant protein, a KRas G12R mutant protein, or a KRas G12V mutant protein)), for example, by detecting a change in the amount of GST-Ras-RBD that binds to the KRas protein following pull-down and antibody detection of the complex. See, e.g., US 2021/0179633.

An exemplary SOS1-catalyzed nucleotide exchange assay protocol follows. GST-KRas G12R (1-169) loaded with GDP nucleotide is mixed with Anti-GST (Cisbio) antibody in assay buffer (20 mM HEPES pH 7.4, 150 mM NaCl, 5 mM MgCl₂, 1 mM DTT, 0.005% NP40, 1% DMSO) to produce a 1.5× solution. 10 μL of the 1.5× KRas-Ab solution is added to wells of a black, low-volume 384-well assay plate. Compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are added to wells using acoustic transfer technology. A 10-point dose response of each compound is performed with a 30 μM top dose. The KRas/Ab-compound mixture is incubated 1 hour at room temperature. A 3× solution of SOS1 (564-1049) and EDA-GTP-DY-647P1 (Jena Bioscience) is prepared in assay buffer. 5 μL of the SOS1-labeled GTP solution is added to the wells to initiate the nucleotide exchange reaction. The final concentration of KRas G12R and SOS1 are 10 nM and 200 nM, respectively. Time resolved fluorescence is read on a PHERAstar plate reader equipped with a filter module with excitation=337 nm and emission 1=620 nm, emission 2=665 nm. The HTRF signal is calculated as the ratio of fluorescence intensity [emission 665 nm]/[emission 620 nm]. IC₅₀ values are calculated using a four-parameter, variable response sigmoidal dose response curve fit in Graphpad Prism software.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits SOS1-catalyzed exchange of GDP for GTP on the KRas protein with an IC₅₀ of less than 1 μM (e.g., less than 750 nM, less than 500 nM, or less than 200 nM). In some embodiments, the compounds inhibit SOS1-catalyzed exchange of GDP for GTP on the KRas protein with an IC₅₀ of less than 200 nM (e.g., less than 150 nM, less than 200 nM, less than 100 nM, less than 10 nM, less than 1 nM, less than 0.1 nM, or less than 0.01 nM). For example, the compounds can inhibit SOS1-catalyzed exchange of GDP for GTP on the KRas protein with an IC₅₀ of 0.001 nM to 500 nM, 0.005 nM to 100 nM, 0.025 nM to 100 nM, 0.1 nM to 50 nM, or 0.1 nM to 10 nM.

Additional assays for evaluating the potency of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can also include, for example, a RAF kinase interaction assay. Such assays can be used to measure the affinity of KRas:nucleotide complexes for the Ras Binding Domain (RBD) of a RAF protein kinase (e.g., as impacted by a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof). For example, FLAG tagged KRas protein can be preloaded with the GTP analogue GppNHp and then incubated with biotinylated Raf-RBD to form complexes. A competition assay can then be performed by adding untagged KRas protein preloaded with GppNHp, which had been preloaded with various test molecules, over a range of concentrations. The proximity-dependent signal after addition of streptavidin donor and anti-flag acceptor beads (e.g., ALPHASCREEN® beads) can be measured to determine the affinity of the KRas protein for the Raf kinase. See, e.g., Hunter et al. Mol Cancer Res. 2015; 13(9):1325-35; Lim et al. Angew Chem Int Ed Engl. 2014; 53(1): 199-204; and Durrant, et al. Molecular Cancer Therapeutics 20.9 (2021): 1743-1754. As another example, for compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, that may bind selectively to the GTP-state, His-tagged KRas protein can be preloaded with the GTP analogue GppNHp and then incubated with a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, to form complexes. A competition assay can then be performed by adding Raf-RBD. The proximity-dependent signal after addition of Alpha detection reagents, compared to the signal from the same experiment using GDP instead of GppNHP, can be used to determine an IC₅₀ value. See, e.g., International Publication No. WO 2021/085653. It will be understood that in many cases, tagging technologies (e.g., FLAG tag, His tag, biotinylation) may be altered in an assay by one of skill in the art. In some embodiments, a RAF kinase interaction assay can be coupled with a nucleotide exchange assay; for example, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can be incubated with a KRas protein (e.g., a dysregulated KRas protein, e.g., a mutant KRas protein (e.g., a KRas G12D mutant protein, a KRas G12R mutant protein, or a KRas G12V mutant protein)) and GDP, then GTP (and optionally, a GEF such as SOS1) can be introduced. Then, RAF (e.g., cRAF) acceptor beads (e.g., GST-tagged acceptor beads) can be incubated with the KRas mixture, followed by introduction of donor beads (e.g., glutathione donor beads) and measurement using ALPHASCREEN® technology. As an alternative to ALPHASCREEN® technology, any appropriate FRET pair can be used to perform homogenous time resolved fluorescence. See, e.g., U.S. Publication Nos. 2018/0334454 and 2021/0230142. Another exemplary assay to measure the affinity of KRas:nucleotide complex for a RBD is to incubate cells with a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, lyse the cells, then pull down non-RBD-bound KRas using an immobilized RBD. See, e.g., U.S. Publication No. 2019/0233440. As another example, the effect of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, on the interaction between KRas and Raf-RBD can be evaluated using HiBiT and/or NANOBIT™ technology, wherein two parts of an enzyme are fused to or inserted into two proteins of interest (e.g., KRas and Raf-RBD); when the two proteins of interest are in proximity, the two parts of the enzyme complement each other to complete an enzyme that has signaling activity (e.g., that produces luminescence). In some such assays, the affinity of the two parts of the enzyme can be tuned, for example, to reduce or eliminate signal based on proximity driven by the two parts of the enzyme. See, e.g., Schwinn, et al. ACS Chemical Biology 13.2 (2018): 467-474. Similarly, the effect of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, on the interaction between KRas and Raf-RBD can be evaluated using NANOBRET™ technology, wherein two parts of signaling system (e.g., a protein and a ligand) are fused to or inserted into two proteins of interest (e.g., KRas and Raf-RBD); when the two proteins of interest are in proximity, the two parts of the signaling system have signaling activity (e.g., producing fluorescence). See, e.g., Durrant, et al. Molecular Cancer Therapeutics 20.9 (2021): 1743-1754. In some embodiments, a RAF kinase interaction assay can be used to determine if a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is selective for a KRas protein (e.g., a dysregulated KRas protein, e.g., a mutant KRas protein (e.g., a KRas G12D mutant protein, a KRas G12R mutant protein, or a KRas G12V mutant protein)) in the GDP-bound state or the GTP-bound state.

Inhibition of the interaction between the KRas protein and Raf-RBD by compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, can be measured using methods known in the art (e.g., using one or more methods described herein (e.g., using methods described in Example B3 herein)).

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, modulates the interaction between the KRas protein and one or more Raf proteins. In some embodiments, the compounds inhibit the interaction between the KRas protein and Raf-RBD with an IC₅₀ of less than 1 μM (e.g., less than 750 nM, less than 500 nM, or less than 200 nM). In some embodiments, the compounds inhibit the interaction between the KRas protein and Raf-RBD with an IC₅₀ of less than 200 nM (e.g., e.g., less than 150 nM, less than 200 nM, less than 100 nM, less than 10 nM, less than 1 nM, less than 0.1 nM, or less than 0.01 nM). For example, the compounds inhibit the interaction between the KRas protein and Raf-RBD with an IC₅₀ from 0.001 nM to 500 nM, from 0.005 nM to 100 nM, from 0.025 nM to 100 nM, from 0.1 nM to 50 nM, or from 0.1 nM to 10 nM.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, inhibits the interaction between the KRas protein and Raf-RBD with an IC₅₀ of less than 1 μM in the absence of cyclophilin A (e.g., less than 750 nM, less than 500 nM, or less than 200 nM). In some embodiments, the compounds inhibit the interaction between the KRas protein and Raf-RBD with an IC₅₀ of less than 200 nM in the absence of cyclophilin A (e.g., e.g., less than 150 nM, less than 200 nM, less than 100 nM, less than 10 nM, less than 1 nM, less than 0.1 nM, or less than 0.01 nM). For example, the compounds inhibit the interaction between the KRas protein and Raf-RBD with an IC₅₀ from 0.001 nM to 500 nM, from 0.005 nM to 100 nM, from 0.025 nM to 100 nM, from 0.1 nM to 50 nM, or from 0.1 nM to 10 nM in the absence of cyclophilin A.

Another exemplary assay for evaluating the potency of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, includes measuring the phosphorylation of a downstream kinase, such as ERK (e.g., ERK1 and/or ERK2) or MEK. Such assays can be used to measure the inhibition of KRas signaling activity, for instance, in a cell line (e.g., PSN1, HUPT3, KP2, CAL62, TCCPAN2, PK8, PATC50, H358, MIAPaCa-2, CALU1 AGS, A427, ASPC1, HKA1, KMS20, A549, LS123, HTK, H727, HCT116, H460, A375, NCI-H1993, PC9, MKN1, NCI-H211, NCI-H424, and/or NCI-H526).

For example, cells can be contacted with a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, for a period of time, then lysed or permeabilized, and total ERK or MEK and phosphoERK or phosphoMEK content can be determined (e.g., using antibodies, or a kit, such as Invitrogen InstantOne ERK1/ERK2 (Phospho) [pT202/pY204]/[pT185/pY187] ELISA or MesoScale Discovery p/t ERK1/2). In some embodiments, multiple concentrations of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can be used to construct a dose response curve. In some embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, inhibit ERK phosphorylation in a cell line expressing a KRas protein (e.g., a mutant KRas protein (e.g., a KRas G12D mutant protein, a KRas G12R mutant protein, or a KRas G12V mutant protein)) with an IC₅₀ of less than 1 μM (e.g., less than 750 nM, less than 500 nM, or less than 200 nM). In some embodiments, the compounds inhibit ERK phosphorylation in a cell line expressing a KRas protein (e.g., a mutant KRas protein (e.g., a KRas G12D mutant protein, a KRas G12R mutant protein, or a KRas G12V mutant protein)) with an IC₅₀ of less than 200 nM (e.g., less than 150 nM, less than 200 nM, less than 100 nM, less than 10 nM, less than 1 nM). For example, the compounds can inhibit ERK phosphorylation in a cell line expressing a KRas protein (e.g., a mutant KRas protein (e.g., a KRas G12D mutant protein, a KRas G12R mutant protein, or a KRas G12V mutant protein)) with an IC₅₀ from 0.1 nM to 100 nM, from 0.1 nM to 50 nM, from 1 nM to 50 nM, or from 1 nM to 20 nM.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can selectively inhibit one or more mutant KRas proteins over wild type KRas protein. The selectivity between wild type KRas protein and a mutant KRas protein as described herein can be measured using cellular proliferation assays where cell proliferation is dependent on signaling activity. For example, HEK293 cells transfected with a suitable version of wild type KRas, or HEK293 cells transfected with KRas containing one or more mutations as described herein (e.g., a G12D mutation, a G12R mutation, or a G12V mutation) can be used. Proliferation assays are performed at a range of inhibitor concentrations (e.g., 10 μM, 3 μM, 1.1 μM, 330 nM, 110 nM, 33 nM, 11 nM, 3 nM, 1 nM) and an EC₅₀ is calculated.

See also the assays described in International Publication Nos. WO 2021/120890; WO 2021/041671; and U.S. Publication Nos. US 2021/0130369; US 2021/0179633; US 2018/0334454; and US 2021/0122764.

The pharmacokinetic parameters of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can be evaluated in an animal model, for instance, a mouse model, a rat model, a dog model, or a nonhuman primate (e.g., cynomolgus monkey) model. Pharmacokinetics parameters, including clearance (CL), volume of distribution (V_{d}), maximum plasma concentration (Cₘₐₓ), time of maximum plasma concentration (tₘₐₓ), half-life (t_{1/2}), area under the curve (AUC), and oral bioavailability (% F) can be calculated using, e.g., a non-compartmental model. In some embodiments, a reference compound (e.g., a first KRas inhibitor (e.g., MRTX1133)) may be used as a comparator. See, e.g., Example 3 (“Pharmacokinetic experiments in mice”) of International Publication No. WO 2023/098425.

Certain pharmacokinetic parameters of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can be evaluated in hepatocytes, such as in mouse, rat, dog, nonhuman primate (e.g., cynomolgus monkey), or human hepatocytes. Pharmacokinetics parameters, including clearance (CL) and half-life (t_{1/2}), can be calculated. In some embodiments, a reference compound (e.g., a first KRas inhibitor (e.g., MRTX1133)) may be used as a comparator. See, e.g., Example VI (“Liver microsomal metabolically stability”) of International Publication No. WO 2023/284881.

In some embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, can exhibit potent and selective inhibition of a dysregulated KRas protein (e.g., a wild-type KRas protein and/or a mutant KRas protein (e.g., a KRas G12D mutant protein, a KRas G12R mutant protein, and/or a KRas G12V mutant protein (e.g., a KRas G12D mutant protein and/or a KRas G12V mutant protein))). In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutically acceptable salt thereof, can selectively inhibit a dysregulated KRas protein (e.g., a wild-type KRas protein and/or a mutant KRas protein (e.g., a KRas G12D mutant protein, a KRas G12R mutant protein, and/or a KRas G12V mutant protein (e.g., a KRas G12D mutant protein and/or a KRas G12V mutant protein))) over another GTPase or non-GTPase target. In some embodiments, the compounds provided herein can exhibit nanomolar potency against a KRas protein (e.g., a wild-type KRas protein and/or a mutant KRas protein (e.g., a KRas G12D mutant protein, a KRas G12R mutant protein, and/or a KRas G12V mutant protein (e.g., a KRas G12D mutant protein and/or a KRas G12V mutant protein))) with minimal activity against related GTPases (e.g., wild type NRas protein, and/or wild type HRas protein).

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit greater inhibition of a KRas protein (e.g., a wild-type KRas protein and/or a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative to inhibition of a related GTPase (e.g., wild type NRas protein, and/or wild type HRas protein). In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit at least 2-fold, 3-fold, 5-fold, 10-fold, 25-fold, 50-fold, or 100-fold greater inhibition of a KRas protein (e.g., a wild-type KRas protein and/or a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative to inhibition of a related GTPase (e.g., wild type NRas protein, and/or wild type HRas protein). In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit up to 10000-fold greater inhibition of a KRas protein (e.g., a wild-type KRas protein and/or a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative to inhibition of a related GTPase (e.g., wild type NRas protein, and/or wild type HRas protein).

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit from about 2-fold to about 10-fold greater inhibition of a KRas protein (e.g., a wild-type KRas protein and/or a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative to inhibition of a related GTPase (e.g., wild type NRas protein, and/or wild type HRas protein). In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit from about 10-fold to about 100-fold greater inhibition of a KRas protein (e.g., a wild-type KRas protein and/or a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative to inhibition of a related GTPase (e.g., wild type NRas protein, and/or wild type HRas protein). In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit from about 100-fold to about 1000-fold greater inhibition of a KRas protein (e.g., a wild-type KRas protein and/or a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative inhibition of a related GTPase (e.g., wild type NRas protein, and/or wild type HRas protein). In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit from about 1000-fold to about 10000-fold greater inhibition of a KRas protein (e.g., a wild-type KRas protein and/or a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative to inhibition of a related GTPase (e.g., wild type NRas protein, and/or wild type HRas protein).

In some embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, can exhibit potent and selective inhibition of a dysregulated KRas protein (e.g., a mutant KRas protein (e.g., a KRas G12D mutant protein, a KRas G12R mutant protein, and/or a KRas G12V mutant protein (e.g., a KRas G12D mutant protein and/or a KRas G12V mutant protein))). In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutically acceptable salt thereof, can selectively inhibit a dysregulated KRas protein (e.g., a mutant KRas protein (e.g., a KRas G12D mutant protein, a KRas G12R mutant protein, and/or a KRas G12V mutant protein (e.g., a KRas G12D mutant protein and/or a KRas G12V mutant protein))) over another GTPase or non-GTPase target. In some embodiments, the compounds provided herein can exhibit nanomolar potency against a mutant KRas protein (e.g., a KRas G12D mutant protein, a KRas G12R mutant protein, and/or a KRas G12V mutant protein (e.g., a KRas G12D mutant protein and/or a KRas G12V mutant protein))) with minimal activity against related GTPases (e.g., wild type KRas protein, wild type NRas protein, and/or wild type HRas protein).

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit greater inhibition of a mutant KRas protein (e.g., a KRas G12D mutant protein, a KRas G12R mutant protein, and/or a KRas G12V mutant protein (e.g., a KRas G12D mutant protein and/or a KRas G12V mutant protein)) relative to inhibition of wild type KRas protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit at least 2-fold, 3-fold, 5-fold, 10-fold, 25-fold, 50-fold, or 100-fold greater inhibition of a mutant KRas protein (e.g., a KRas G12D mutant protein, a KRas G12R mutant protein, and/or a KRas G12V mutant protein (e.g., a KRas G12D mutant protein and/or a KRas G12V mutant protein)) relative to inhibition of wild type KRas protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit up to 10000-fold greater inhibition of a mutant KRas protein (e.g., a KRas G12D mutant protein, a KRas G12R mutant protein, and/or a KRas G12V mutant protein (e.g., a KRas G12D mutant protein and/or a KRas G12V mutant protein)) relative to inhibition of wild type KRas protein.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit from about 2-fold to about 10-fold greater inhibition of a mutant KRas protein (e.g., a KRas G12D mutant protein, a KRas G12R mutant protein, and/or a KRas G12V mutant protein (e.g., a KRas G12D mutant protein and/or a KRas G12V mutant protein)) relative to inhibition of wild type KRas protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit from about 10-fold to about 100-fold greater inhibition of a mutant KRas protein (e.g., a KRas G12D mutant protein, a KRas G12R mutant protein, and/or a KRas G12V mutant protein (e.g., a KRas G12D mutant protein and/or a KRas G12V mutant protein)) relative to inhibition of wild type KRas protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit from about 100-fold to about 1000-fold greater inhibition of a mutant KRas protein (e.g., a KRas G12D mutant protein, a KRas G12R mutant protein, and/or a KRas G12V mutant protein (e.g., a KRas G12D mutant protein and/or a KRas G12V mutant protein)) relative to inhibition of wild type KRas protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit from about 1000-fold to about 10000-fold greater inhibition of a mutant KRas protein (e.g a KRas G12D mutant protein, a KRas G12R mutant protein, and/or a KRas G12V mutant protein (e.g., a KRas G12D mutant protein and/or a KRas G12V mutant protein)) relative to inhibition of wild type KRas protein.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (TT-f), (II-g), or (II-h)), or Formula (Aa)), or a pharmaceutically acceptable salt thereof, can exhibit nanomolar potency against a dysregulated KRas protein (e.g., a mutant KRas protein (e.g., a KRas G12D mutant protein, a KRas G12R mutant protein, and/or a KRas G12V mutant protein (e.g., a KRas G12D mutant protein and/or a KRas G12V mutant protein))) with minimal activity against wild type NRas protein, and/or wild type HRas protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit greater inhibition of a dysregulated KRas protein (e.g., a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative to inhibition of wild type NRas protein, and/or wild type HRas protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit at least 2-fold, 3-fold, 5-fold, 10-fold, 25-fold, 50-fold or 100-fold greater inhibition of a dysregulated KRas protein (e.g., a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative to inhibition of wild type NRas protein, and/or wild type HRas protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit up to 1000-fold greater inhibition of a dysregulated KRas protein (e.g., a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative to inhibition of wild type NRas protein, and/or wild type HRas protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit up to 10000-fold greater inhibition of a dysregulated KRas protein (e.g., a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative to inhibition of wild type NRas protein, and/or wild type HRas protein.

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (TT-f), (II-g), or (II-h)), or Formula (Aa)), or a pharmaceutically acceptable salt thereof, can exhibit from about 2-fold to about 10-fold greater inhibition of a dysregulated KRas protein (e.g., a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative to inhibition of wild type NRas protein, and/or wild type HRas protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit from about 10-fold to about 100-fold greater inhibition of a dysregulated KRas protein (e.g., a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative to inhibition of wild type HRas protein and/or wild type NRas protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit from about 100-fold to about 1000-fold greater inhibition of a dysregulated KRas protein (e.g., a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative to inhibition of wild type NRas protein, and/or wild type HRas protein. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can exhibit from about 1000-fold to about 10000-fold greater inhibition of a dysregulated KRas protein (e.g., a mutant KRas protein (e.g., a KRas G12D mutant protein, a KRas G12R mutant protein, and/or a KRas G12V mutant protein (e.g., a KRas G12D mutant protein and/or a KRas G12V mutant protein))) relative to inhibition of wild type NRas protein, and/or wild type HRas protein.

Compounds Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are useful for treating diseases and disorders including cardiovascular disease (e.g., arteriovenous malformations or Noonan syndrome), endometriosis, an inflammatory and/or autoimmune disease (e.g., a nonmalignant syndrome of autoimmunity and abnormal leukocyte homeostasis), and proliferative disorders such as cancers, including hematological cancers and solid tumors (e.g., advanced solid tumors). In some embodiments, the diseases and disorders are KRas-associated diseases and disorders (e.g., mutant KRas-associated diseases or disorders (e.g., KRas G12D-, KRas G12R-, or G12V-associated diseases or disorders)).

In certain embodiments, compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are useful for preventing diseases and disorders as defined herein (for example, a cardiovascular disease, endometriosis, and an inflammatory and/or autoimmune disease, or cancer).

In some embodiments of any of the methods or uses described herein, the inflammatory and/or autoimmune disease is RAS-associated autoimmune leukoproliferative disease. See, e.g., Niemela et al. Blood. 2011; 117(10):2883-6.

In some embodiments, the subject has been identified or diagnosed as having a cancer with a KRas dysregulation (e.g., a KRas mutation or amplification) (e.g., as determined using a regulatory agency-approved, e.g., FDA-approved, assay or kit). In some embodiments, the subject has a cancer (e.g., a tumor sample) that has a KRas dysregulation (e.g., a KRas mutation or amplification) (e.g., as determined using a regulatory agency-approved assay or kit). The subject can be a subject with a cancer (e.g., one or more tumor samples) that is positive for a KRas dysregulation (e.g., a KRas mutation or amplification) (e.g., identified as positive using a regulatory agency-approved, e.g., FDA-approved, assay or kit). The subject can be a subject whose cancer (e.g., a tumor sample) has a KRas dysregulation (e.g., a KRas mutation or amplification) (e.g., where the cancer (e.g., tumor sample) is identified as such using a regulatory agency-approved, e.g., FDA-approved, kit or assay). In some embodiments, the subject is suspected of having a mutant KRas-associated cancer. In some embodiments, the subject has a clinical record indicating that the subject has a cancer (e.g., a tumor sample) that has a KRas dysregulation (e.g., a KRas mutation or amplification) (and optionally the clinical record indicates that the subject should be treated with any of the compounds and/or compositions provided herein).

In some such embodiments, the cancer (e.g., a tumor sample) has a KRas mutation selected from the group consisting of: a KRas G12X mutation, a KRas G13X mutation, and a KRas Q61X mutation. In some embodiments, a KRas mutation is selected from the group consisting of: a KRas G12A mutation, a KRas G12C mutation, a KRas G12D mutation, a KRas G12R mutation, a KRas G12S mutation, a KRas G12V mutation, a KRas G13C mutation, a KRas G13D mutation, a KRas G13V mutation, a KRas Q61E mutation, a KRas Q61H mutation, a KRas Q61K mutation, a KRas Q61L mutation, a KRas Q61P mutation, and a KRas Q61R mutation. In some embodiments, a KRas mutation is selected from the group consisting of: a KRas G12A mutation, a KRas G12C mutation, a KRas G12D mutation, a KRas G12R mutation, a KRas G12S mutation, and a KRas G12V mutation. In some embodiments, the cancer (e.g., a tumor sample) has a KRas mutation selected from the group consisting of: a KRas G12A mutation, a KRas G12D mutation, a KRas G12R mutation, and a KRas G12V mutation.

In some embodiments, the cancer (e.g., a tumor sample) has a KRas G12D mutation, a KRas G12R mutation, or KRas G12V mutation (e.g., a KRas G12D mutation or a KRas G12V mutation). In some embodiments, the cancer (e.g., a tumor sample) has a KRas G12D mutation or a KRas G12V mutation. In some embodiments, the cancer (e.g., a tumor sample) has a KRas G12D mutation. In some embodiments, the cancer (e.g., a tumor sample) has a KRas G12R mutation. In some embodiments, the cancer (e.g., a tumor sample) has a KRas G12V mutation.

The term “KRas-associated cancer” as used herein refers to cancers associated with or having a dysregulation of a KRAS gene, a KRas protein, or the expression or activity or level of any (e.g., one or more) of the same (e.g., any of the types of dysregulations of a KRAS gene, a KRas protein, or the expression or activity or level of any of the same described herein). Non-limiting examples of a KRas-associated cancer are described herein.

The term “mutant KRas-associated cancer” as used herein refers to cancers associated with or having a KRas mutation (e.g., a KRAS gene having a mutation corresponding to a mutation in a KRas protein and/or a KRas protein having a mutation). Non-limiting examples of a mutant KRas-associated cancer are described herein.

The term “KRas G12X-associated cancer” as used herein refers to cancers associated with or having a KRas G12X mutation (e.g., a KRAS gene having a mutation corresponding to a G12X mutation in a KRas protein and/or a KRas protein having a G12X mutation). Non-limiting examples of a KRas G12X-associated cancer are described herein.

The term “KRas G12A-associated cancer” as used herein refers to cancers associated with or having a KRas G12A mutation (e.g., a KRAS gene having a mutation corresponding to a G12A mutation in a KRas protein and/or a KRas protein having a G12A mutation). Non-limiting examples of a KRas G12A-associated cancer are described herein.

The term “KRas G12C-associated cancer” as used herein refers to cancers associated with or having a KRas G12C mutation (e.g., a KRAS gene having a mutation corresponding to a G12C mutation in a KRas protein and/or a KRas protein having a G12C mutation). Non-limiting examples of a KRas G12C-associated cancer are described herein.

The term “KRas G12D-associated cancer” as used herein refers to cancers associated with or having a KRas G12D mutation (e.g., a KRAS gene having a mutation corresponding to a G12D mutation in a KRas protein and/or a KRas protein having a G12D mutation). Non-limiting examples of a KRas G12D-associated cancer are described herein.

The term “KRas G12R-associated cancer” as used herein refers to cancers associated with or having a KRas G12R mutation (e.g., a KRAS gene having a mutation corresponding to a G12R mutation in a KRas protein and/or a KRas protein having a G12R mutation). Non-limiting examples of a KRas G12R-associated cancer are described herein.

The term “KRas G12S-associated cancer” as used herein refers to cancers associated with or having a KRas G12S mutation (e.g., a KRAS gene having a mutation corresponding to a G12S mutation in a KRas protein and/or a KRas protein having a G12S mutation). Non-limiting examples of a KRas G12S-associated cancer are described herein.

The term “KRas G12V-associated cancer” as used herein refers to cancers associated with or having a KRas G12V mutation (e.g., a KRAS gene having a mutation corresponding to a G12V mutation in a KRas protein and/or a KRas protein having a G12V mutation). Non-limiting examples of a KRas G12V-associated cancer are described herein.

The term “KRas G13X-associated cancer” as used herein refers to cancers associated with or having a KRas G13X mutation (e.g., a KRAS gene having a mutation corresponding to a G13X mutation in a KRas protein and/or a KRas protein having a G13X mutation). Non-limiting examples of a KRas G13X-associated cancer are described herein.

The term “KRas G13C-associated cancer” as used herein refers to cancers associated with or having a KRas G13C mutation (e.g., a KRAS gene having a mutation corresponding to a G13C mutation in a KRas protein and/or a KRas protein having a G13C mutation). Non-limiting examples of a KRas G13C-associated cancer are described herein.

The term “KRas G13D-associated cancer” as used herein refers to cancers associated with or having a KRas G13D mutation (e.g., a KRAS gene having a mutation corresponding to a G13D mutation in a KRas protein and/or a KRas protein having a G13D mutation). Non-limiting examples of a KRas G13D-associated cancer are described herein.

The term “KRas G13V-associated cancer” as used herein refers to cancers associated with or having a KRas G13V mutation (e.g., a KRAS gene having a mutation corresponding to a G13V mutation in a KRas protein and/or a KRas protein having a G13V mutation). Non-limiting examples of a KRas G13V-associated cancer are described herein.

The term “KRas Q61X-associated cancer” as used herein refers to cancers associated with or having a KRas Q61X mutation (e.g., a KRAS gene having a mutation corresponding to a Q61X mutation in a KRas protein and/or a KRas protein having a Q61X mutation). Non-limiting examples of a KRas Q61X-associated cancer are described herein.

The term “KRas Q61E-associated cancer” as used herein refers to cancers associated with or having a KRas Q61E mutation (e.g., a KRAS gene having a mutation corresponding to a Q61E mutation in a KRas protein and/or a KRas protein having a Q61E mutation). Non-limiting examples of a KRas Q61E-associated cancer are described herein.

The term “KRas Q61H-associated cancer” as used herein refers to cancers associated with or having a KRas Q61H mutation (e.g., a KRAS gene having a mutation corresponding to a Q61H mutation in a KRas protein and/or a KRas protein having a Q61H mutation). Non-limiting examples of a KRas Q61H-associated cancer are described herein.

The term “KRas Q61K-associated cancer” as used herein refers to cancers associated with or having a KRas Q61K mutation (e.g., a KRAS gene having a mutation corresponding to a Q61K mutation in a KRas protein and/or a KRas protein having a Q61K mutation). Non-limiting examples of a KRas Q61K-associated cancer are described herein.

The term “KRas Q61L-associated cancer” as used herein refers to cancers associated with or having a KRas Q61L mutation (e.g., a KRAS gene having a mutation corresponding to a Q61L mutation in a KRas protein and/or a KRas protein having a Q61L mutation). Non-limiting examples of a KRas Q61L-associated cancer are described herein.

The term “KRas Q61P-associated cancer” as used herein refers to cancers associated with or having a KRas Q61P mutation (e.g., a KRAS gene having a mutation corresponding to a Q61P mutation in a KRas protein and/or a KRas protein having a Q61P mutation). Non-limiting examples of a KRas Q61P-associated cancer are described herein.

The term “KRas Q61R-associated cancer” as used herein refers to cancers associated with or having a KRas Q61R mutation (e.g., a KRAS gene having a mutation corresponding to a Q61R mutation in a KRas protein and/or a KRas protein having a Q61R mutation). Non-limiting examples of a KRas Q61R-associated cancer are described herein.

Such mutations can be associated with the development of a variety of cancers. See, e.g., Hunter et al. Mol Cancer Res. 2015; 13(9):1325-35.

Provided herein are methods of treating a cancer in a subject in need of such treatment, the methods comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition thereof. In some embodiments, the subject is treatment naïve with respect to the cancer. In some embodiments, the subject has received one or more lines of previous therapy for the cancer.

Also provided herein are methods of treating a cancer in a subject in need thereof, the methods comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition thereof, as a monotherapy. In some embodiments, the subject is treatment naïve with respect to the cancer. In some embodiments, the subject has received one or more lines of previous therapy for the cancer.

Provided herein is use of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition thereof, for the treatment of cancer, for example, any of the cancers provided herein.

Provided herein is use of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition thereof, as a medicament for the treatment of cancer, for example, any of the cancers provided herein.

Provided herein is use of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, in the manufacture of a medicament for the treatment of cancer, for example, any of the cancers provided herein.

Provided herein is a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition thereof, for use as a medicament. Also provided herein is a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition thereof, for use as a medicament for the treatment of cancer, for example, any of the cancers provided herein.

Provided herein is a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition thereof, for use in treating a cancer, for example, any of the cancers provided herein.

As used herein, “monotherapy”, when referring to a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, means that the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is the only therapeutic agent or therapy (e.g., anticancer agent or therapy) administered to the subject during the treatment cycle (e.g., no additional targeted therapeutics, anticancer agents, chemotherapeutics, or checkpoint inhibitors are administered to the subject during the treatment cycle). As a person of ordinary skill in the art would understand, monotherapy does not exclude the co-administration of medicaments for the treatment of side effects or general symptoms associated with the cancer or treatment, such as pain, rash, edema, photosensitivity, pruritis, skin discoloration, hair brittleness, hair loss, brittle nails, cracked nails, discolored nails, swollen cuticles, fatigue, weight loss, general malaise, shortness of breath, infection, anemia, or gastrointestinal symptoms, including nausea, diarrhea, and lack of appetite.

As used herein, “the subject has previously received one or more therapeutic agents or therapies for the cancer” means that the subject has been previously administered one or more therapeutic agents or therapies (e.g., anticancer agent or therapy) for the cancer other than a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, during a prior treatment cycle. In some embodiments, the subject cannot tolerate the one or more therapeutic agents or therapies previously administered for the cancer. In some embodiments, the subject did not respond to the one or more therapeutic agents or therapies previously administered for the cancer. In some embodiments, the subject did not adequately respond to one or more therapeutic agents or therapies previously administered for the cancer. In some embodiments, the subject has stopped responding to the one or more therapeutic agents or therapies previously administered for the cancer. In some embodiments, a lack of response, an inadequate response, or a discontinued response can be determined by objective criteria (e.g., tumor volume, or by criteria such as RECIST 1.1). In some embodiments, a lack of response, an inadequate response, or a discontinued response can be determined by the subject's physician.

As used herein, “the subject is treatment naïve with respect to the cancer” means that the subject has not been previously administered one or more therapeutic agents or therapies for the cancer.

For any of the solid tumors described herein, the solid tumors can be primary tumors or metastatic (or secondary) tumors. As used herein, “primary” tumors are those located at the site where the tumor began to grow (i.e., where it originated). As used herein, “metastatic” (or “secondary”) tumors are those that have spread to other parts of body from the original tumor site. In some embodiments, the metastatic or secondary tumors are the same type of cancer as the primary tumor. In some embodiments, the metastatic or secondary tumors are not genetically identical to the primary tumor.

Provided herein is a method of treating a cancer in a in a subject in need of such treatment, the method comprising a) detecting a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification) in a sample from the subject (e.g., detecting a KRAS gene having a mutation corresponding to a mutation in KRas protein and/or detecting a KRas protein having a mutation, a KRAS gene copy number increase, and/or an increase in KRas mRNA or protein expression); and b) administering a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof. Also provided herein is a method of treating a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. For example, provided herein are methods for treating a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))) in a subject in need of such treatment, the methods comprising a) detecting a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification) in a sample from the subject (e.g., detecting a KRAS gene having a mutation corresponding to a mutation in KRas protein and/or detecting a KRas protein having a mutation, a KRAS gene copy number increase, and/or an increase in KRas mRNA or protein expression); and b) administering a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof.

In some embodiments of any of the methods or uses described herein, the cancer (e.g., KRas-associated cancer (e.g., mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer)))) is breast cancer (e.g., breast invasive carcinoma, breast invasive ductal carcinoma), central or peripheral nervous system tissue cancer (e.g., brain cancer (e.g., astrocytoma, glioblastoma, glioma, oligoastrocytoma)), endocrine or neuroendocrine cancer (e.g., adrenal cancer (e.g., adrenocortical carcinoma, pheochromocytoma, paraganglioma), multiple neuroendocrine type I and type II tumors, parathyroid cancer, pituitary tumors, thyroid cancer (e.g., papillary thyroid cancer)), eye cancer (e.g., uveal cancer (e.g., uveal melanoma)), gastrointestinal cancer (e.g., anal cancer, bile duct cancer (e.g., cholangiocarcinoma), colorectal cancer (e.g., colon adenocarcinoma, rectal adenocarcinoma, mucinous adenocarcinoma, mucinous carcinoma), esophageal cancer (e.g., esophageal adenocarcinoma), gallbladder cancer, gastrointestinal carcinoid tumor, gastrointestinal stromal tumor, liver cancer (e.g., hepatocellular carcinoma, intrahepatic bile duct cancer), pancreatic cancer (e.g., pancreatic adenocarcinoma, pancreatic islet cell cancer), small intestine cancer, or stomach cancer (e.g., stomach adenocarcinoma, signet ring cell carcinoma of the stomach)), genitourinary cancer (e.g., bladder cancer (e.g., bladder urothelial carcinoma), kidney cancer (e.g., renal clear cell carcinoma, renal papillary cell carcinoma, kidney chromophobe), prostate cancer (e.g., prostate adenocarcinoma), testicular cancer (e.g., testicular germ cell tumors, seminoma), or ureter cancer), gynecologic cancer (e.g., cervical cancer (e.g., cervical squamous cell carcinoma, endocervical adenocarcinoma, mucinous carcinoma), ovarian cancer (e.g., serous ovarian cancer, ovarian serous cystadenocarcinoma), uterine cancer (e.g., uterine carcinosarcoma, uterine endometrioid carcinoma, uterine serous carcinoma, uterine papillary serous carcinoma, uterine corpus endometrial carcinoma), or vulvar cancer), head and neck cancer (e.g., ear cancer (e.g., middle ear cancer), head and neck squamous cell carcinoma, nasal cavity cancer, oral cancer, pharynx cancer (e.g., hypopharynx cancer, nasopharynx cancer, oropharyngeal cancer), hematological cancer (e.g., leukemia (e.g., chronic lymphocytic leukemia (CLL), acute lymphocytic leukemia (ALL) (e.g., Philadelphia chromosome positive ALL), acute myeloid leukemia (AML) (e.g., acute promyelocytic leukemia (APL)), chronic myeloid leukemia (CML)), lymphoma (e.g., Hodgkin lymphoma (e.g., nodular lymphocyte predominant Hodgkin lymphoma (NLPHL)), non-Hodgkin lymphoma (e.g., Burkitt lymphoma (BL), diffuse large B-cell lymphoma (DLBCL), diffuse histiocytic lymphoma (DHL), follicular lymphoma (FL), intravascular large B-cell lymphoma (IVLBCL), mantle cell lymphoma (MCL), small lymphocytic lymphoma (SLL))), or myeloma (e.g., multiple myeloma)), Li-Fraumeni tumors, mesentery cancer (e.g., omentum cancer, peritoneal cancer), pleural cancer, respiratory cancer (e.g., larynx cancer, lung cancer (e.g., lung squamous cell carcinoma, lung adenocarcinoma, mesothelioma, non-small cell lung cancer (NSCLC)), tracheal cancer), sarcoma (e.g., bone cancer (e.g., osteosarcoma, chondrosarcoma) or soft tissue sarcoma (Ewing sarcoma, leiomyosarcoma, myxofibrosarcoma, rhabdomyosarcoma)), skin cancer (e.g., melanoma), thymus cancer (e.g., thymoma), or a combination thereof.

In some embodiments, the cancer (e.g., KRas-associated cancer (e.g., mutant KRas-associated cancer)) is a hematological cancer, a soft tissue cancer, bile duct cancer, bladder cancer, brain cancer, breast cancer, cervical cancer, colon cancer, endometrial cancer, esophageal cancer, kidney cancer, liver cancer, lung cancer, mucinous carcinoma, rectal cancer, ovarian cancer, pancreatic cancer, prostate cancer, rectal cancer, skin cancer, stomach cancer, testicular cancer, thymus cancer, thyroid cancer, urothelial cancer, or uterine cancer. In some embodiments, the cancer (e.g., a KRas-associated cancer (e.g., a mutant KRas-associated cancer (e.g., a KRas G12X-associated cancer))) is a hematological cancer, bile duct cancer, brain cancer, breast cancer, cervical cancer, colon cancer, endometrial cancer, esophageal cancer, kidney cancer, liver cancer, lung cancer, mucinous carcinoma, pancreatic cancer, prostate cancer, rectal cancer, testicular cancer (e.g., seminoma), skin cancer, stomach cancer, thymus cancer, thyroid cancer, urothelial cancer, or uterine cancer.

In some embodiments, the cancer is a hematological cancer, brain cancer, cervical cancer, colon cancer, endometrial cancer, esophageal cancer, kidney cancer, liver cancer, lung cancer, mucinous carcinoma, pancreatic cancer, prostate cancer, rectal cancer, skin cancer, stomach cancer, thymus cancer, urothelial cancer, or uterine cancer. In some embodiments, the cancer is a KRas G12D-associated cancer.

In some embodiments, the cancer is a hematological cancer, bladder cancer, bile duct cancer, colon cancer, ovarian cancer, pancreatic cancer, prostate cancer, rectal cancer, skin cancer, or testicular cancer. In some embodiments, the cancer is a KRas G12R-associated cancer.

In some embodiments, the cancer is a hematological cancer, bladder cancer, bile duct cancer, brain cancer, breast cancer, cervical cancer, colon cancer, endometrial cancer, esophageal cancer, kidney cancer, liver cancer, lung cancer, mucinous carcinoma, ovarian cancer, pancreatic cancer, rectal cancer, skin cancer, stomach cancer, testicular cancer (e.g., seminoma), thymus cancer, or uterine cancer. In some embodiments, the cancer is a G12V-associated cancer.

In some embodiments, the cancer (e.g., a KRas-associated cancer (e.g., a mutant KRas-associated cancer (e.g., a KRas G13X-associated cancer))) is a hematological cancer, a soft tissue cancer, cervical cancer, colon cancer, endometrial cancer, liver cancer, lung cancer, pancreatic cancer, rectal cancer, skin cancer, stomach cancer, or urothelial cancer. In some embodiments, the cancer (e.g., a KRas-associated cancer (e.g., a mutant KRas-associated cancer (e.g., a KRas Q61X-associated cancer)) is bladder cancer, colon cancer, lung cancer, ovarian cancer, rectal cancer, thyroid cancer, or uterine cancer. In some embodiments, the cancer (e.g., a KRas-associated cancer (e.g., a cancer associated with KRas amplification (e.g., a cancer associated with wild-type KRas amplification)) is colorectal cancer, gastric cancer, gastroesophageal cancer, head and neck squamous carcinoma, or lung cancer (e.g., NSCLC). See, e.g., the public database cBioPortal.

In some embodiments, the cancer (e.g., a KRas-associated cancer (e.g., mutant KRas-associated cancer)) is pancreatic cancer or metastatic pancreatic cancer. In some embodiments, the cancer (e.g., a KRas-associated cancer (e.g., mutant KRas-associated cancer)) is pancreatic ductal adenocarcinoma (PDAC). In some such embodiments, the pancreatic cancer is a KRas G12R-associated cancer.

In some embodiments, the cancer (e.g., a KRas-associated cancer (e.g., mutant KRas-associated cancer)) is advanced-stage lung adenocarcinoma.

In some embodiments, the cancer is a solid tumor. In some embodiments, the solid tumor has a KRas dysregulation (e.g., a KRas mutation or amplification). For example, the solid tumor has a KRas mutation selected from the group consisting of: a KRas G12A mutation, a KRas G12C mutation, a KRas G12D mutation, a KRas G12R mutation, a KRas G12S mutation, and a KRas G12V mutation. In some embodiments, the solid tumor has a KRas G12D mutation, a KRas G12R mutation, or a KRas G12V mutation. In some embodiments, the solid tumor has a KRas G12D mutation or a KRas G12V mutation. In some embodiments, the solid tumor has a KRas G12D mutation. In some embodiments, the solid tumor has a KRas G12R mutation. In some embodiments, the solid tumor has a KRas G12V mutation.

Also provided herein is a method of treating a solid tumor in a subject in need thereof, the method comprising: (a) detecting a KRas dysregulation (e.g., a KRas mutation or amplification) in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Also provided herein is a method of treating a solid tumor in a subject in need thereof, the method comprising: (a) detecting a a KRas G12A mutation, a KRas G12C mutation, a KRas G12D mutation, a KRas G12R mutation, a KRas G12S mutation, or a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a solid tumor in a subject in need thereof, the method comprising: (a) detecting a KRas G12D or a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a solid tumor in a subject in need thereof, the method comprising: (a) detecting a KRas G12D mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a solid tumor in a subject in need thereof, the method comprising: (a) detecting a KRas G12R mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a solid tumor in a subject in need thereof, the method comprising: (a) detecting a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein.

In some embodiments, the cancer is a bladder cancer. In some embodiments, the bladder cancer has a KRas dysregulation (e.g., a KRas mutation or amplification). For example, the bladder cancer has a KRas mutation selected from the group consisting of: a KRas G12C mutation, a KRas G12D mutation, a KRas G12R mutation, a KRas G12V mutation, a KRas G13D mutation, and a KRas Q61H mutation. In some embodiments, the bladder cancer has a KRas G12D mutation, a KRas G12R mutation, or a KRas G12V mutation. In some embodiments, the bladder cancer has a KRas G12D mutation or a KRas G12V mutation. In some embodiments, the bladder cancer has a KRas G12D mutation. In some embodiments, the bladder cancer has a KRas G12R mutation. In some embodiments, the bladder cancer has a KRas G12V mutation.

Also provided herein is a method of treating a bladder cancer in a subject in need thereof, the method comprising: (a) detecting a KRas dysregulation (e.g., a KRas mutation or amplification) in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Also provided herein is a method of treating a bladder cancer in a subject in need thereof, the method comprising: (a) detecting a KRas G12C mutation, a KRas G12D mutation, a KRas G12R mutation, a KRas G12V mutation, a KRas G13D mutation, or a KRas Q61H mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a bladder cancer in a subject in need thereof, the method comprising: (a) detecting a KRas G12D or a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a bladder cancer in a subject in need thereof, the method comprising: (a) detecting a KRas G12D mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a bladder cancer in a subject in need thereof, the method comprising: (a) detecting a KRas G12R mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a bladder cancer in a subject in need thereof, the method comprising: (a) detecting a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein.

In some embodiments, the cancer is a cervical cancer. In some embodiments, the cervical cancer has a KRas dysregulation (e.g., a KRas mutation or amplification). For example, the cervical cancer has a KRas mutation selected from the group consisting of: a KRas G12C mutation, a KRas G12D mutation, a KRas G12V mutation, and a KRas G13D mutation. In some embodiments, the cervical cancer has a KRas G12D mutation or a KRas G12V mutation. In some embodiments, the cervical cancer has a KRas G12D mutation. In some embodiments, the cervical cancer has a KRas G12V mutation.

Also provided herein is a method of treating a cervical cancer in a subject in need thereof, the method comprising: (a) detecting a KRas dysregulation (e.g., a KRas mutation or amplification) in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Also provided herein is a method of treating a cervical cancer in a subject in need thereof, the method comprising: (a) detecting a KRas G12C mutation, a KRas G12D mutation, a KRas G12V mutation, or a KRas G13D mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a cervical cancer in a subject in need thereof, the method comprising: (a) detecting a KRas G12D mutation or a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a cervical cancer in a subject in need thereof, the method comprising: (a) detecting a KRas G12D mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a cervical cancer in a subject in need thereof, the method comprising: (a) detecting a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein.

In some embodiments, the cancer is a colorectal cancer. In some embodiments, the colorectal cancer has a KRas dysregulation (e.g., a KRas mutation or amplification). For example, the colorectal cancer has a KRas mutation selected from the group consisting of: a KRas G12A mutation, a KRas G12C mutation, a KRas G12D mutation, a KRas G12R mutation, a KRas G12S mutation, a KRas G12V mutation, a KRas G13C mutation, a KRas G13D mutation, a KRas G13V mutation, a KRas Q61E mutation, a KRas Q61H mutation, a KRas Q61K mutation, a KRas Q61L mutation, a KRas Q61P mutation, and a KRas Q61R mutation. In some embodiments, the colorectal cancer has a KRas G12D mutation, a KRas G12R mutation, or a KRas G12V mutation. In some embodiments, the colorectal cancer has a KRas G12D mutation or a KRas G12V mutation. In some embodiments, the colorectal cancer has a KRas G12D mutation. In some embodiments, the colorectal cancer has a KRas G12V mutation.

Also provided herein is a method of treating a colorectal cancer in a subject in need thereof, the method comprising: (a) detecting a KRas dysregulation (e.g., a KRas mutation or amplification) in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Also provided herein is a method of treating a colorectal cancer in a subject in need thereof, the method comprising: (a) detecting a KRas G12A mutation, a KRas G12C mutation, a KRas G12D mutation, a KRas G12R mutation, a KRas G12S mutation, a KRas G12V mutation, a KRas G13C mutation, a KRas G13D mutation, a KRas G13V mutation, a KRas Q61E mutation, a KRas Q61H mutation, a KRas Q61K mutation, a KRas Q61L mutation, a KRas Q61P mutation, or a KRas Q61R mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a colorectal cancer in a subject in need thereof, the method comprising: (a) detecting a KRas G12D mutation or a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a colorectal cancer in a subject in need thereof, the method comprising: (a) detecting a KRas G12D mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a colorectal cancer in a subject in need thereof, the method comprising: (a) detecting a KRas G12R mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a colorectal cancer in a subject in need thereof, the method comprising: (a) detecting a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein.

In some embodiments, the cancer is an endometrial cancer. In some embodiments, the endometrial cancer has a KRas dysregulation (e.g., a KRas mutation or amplification). For example, the endometrial cancer has a KRas mutation selected from the group consisting of: a KRas G12A mutation, a KRas G12C mutation, a KRas G12D mutation, a KRas G12S mutation, a KRas G12V mutation, a KRas G13C mutation, a KRas G13D mutation, a KRas G13V mutation, a KRas Q61H mutation, and a KRas Q61L mutation. In some embodiments, the endometrial cancer has a KRas G12D mutation or a KRas G12V mutation. In some embodiments, the endometrial cancer has a KRas G12D mutation. In some embodiments, the endometrial cancer has a KRas G12V mutation.

Also provided herein is a method of treating an endometrial cancer in a subject in need thereof, the method comprising: (a) detecting a KRas dysregulation (e.g., a KRas mutation or amplification) in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Also provided herein is a method of treating an endometrial cancer in a subject in need thereof, the method comprising: (a) detecting a KRas G12A mutation, a KRas G12C mutation, a KRas G12D mutation, a KRas G12S mutation, a KRas G12V mutation, a KRas G13C mutation, a KRas G13D mutation, a KRas G13V mutation, a KRas Q61H mutation, or a KRas Q61L mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating an endometrial cancer in a subject in need thereof, the method comprising: (a) detecting a KRas G12D mutation or a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating an endometrial cancer in a subject in need thereof, the method comprising: (a) detecting a KRas G12D mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating an endometrial cancer in a subject in need thereof, the method comprising: (a) detecting a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein.

In some embodiments, the cancer is an esophageal or stomach cancer. In some embodiments, the esophageal or stomach cancer has a KRas dysregulation (e.g., a KRas mutation or amplification). For example, the esophageal or stomach cancer has a KRas mutation selected from the group consisting of: a KRas G12C mutation, a KRas G12D mutation, a KRas G12S mutation, a KRas G12V mutation, a KRas G13C mutation, a KRas G13D mutation, and a KRas Q61H mutation. In some embodiments, the esophageal or stomach cancer has a KRas G12D mutation or a KRas G12V mutation. In some embodiments, the esophageal or stomach cancer has a KRas G12D mutation. In some embodiments, the esophageal or stomach cancer has a KRas G12V mutation.

Also provided herein is a method of treating an esophageal or stomach cancer in a subject in need thereof, the method comprising: (a) detecting a KRas dysregulation (e.g., a KRas mutation or amplification) in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Also provided herein is a method of treating an esophageal or stomach cancer in a subject in need thereof, the method comprising: (a) detecting a KRas G12C mutation, a KRas G12D mutation, a KRas G12S mutation, a KRas G12V mutation, a KRas G13C mutation, a KRas G13D mutation, or a KRas Q61H mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating an esophageal or stomach cancer in a subject in need thereof, the method comprising: (a) detecting a KRas G12D mutation or a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating an esophageal or stomach cancer in a subject in need thereof, the method comprising: (a) detecting a KRas G12D mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating an esophageal or stomach cancer in a subject in need thereof, the method comprising: (a) detecting a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein.

In some embodiments, the cancer is a leukemia. In some embodiments, the leukemia has a KRas dysregulation (e.g., a KRas mutation or amplification). For example, the leukemia has a KRas mutation selected from the group consisting of: a KRas G12A mutation, a KRas G12C mutation, a KRas G12D mutation, a KRas G12R mutation, a KRas G12S mutation, a KRas G12V mutation, a KRas G13C mutation, a KRas G13D mutation, a KRas G13V mutation, a KRas Q61E mutation, a KRas Q61H mutation, a KRas Q61K mutation, a KRas Q61L mutation, a KRas Q61P mutation, and a KRas Q61R mutation. In some embodiments, the leukemia has a KRas G12D mutation, a KRas G12R mutation, or a KRas G12V mutation. In some embodiments, the leukemia has a KRas G12D mutation or a KRas G12V mutation. In some embodiments, the leukemia has a KRas G12D mutation. In some embodiments, the leukemia has a KRas G12R mutation. In some embodiments, the leukemia has a KRas G12V mutation.

Also provided herein is a method of treating a leukemia in a subject in need thereof, the method comprising: (a) detecting a KRas dysregulation (e.g., a KRas mutation or amplification) in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Also provided herein is a method of treating a leukemia in a subject in need thereof, the method comprising: (a) detecting a KRas G12A mutation, a KRas G12C mutation, a KRas G12D mutation, a KRas G12R mutation, a KRas G12S mutation, a KRas G12V mutation, a KRas G13C mutation, a KRas G13D mutation, a KRas G13V mutation, a KRas Q61E mutation, a KRas Q61H mutation, a KRas Q61K mutation, a KRas Q61L mutation, a KRas Q61P mutation, or a KRas Q61R mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a leukemia in a subject in need thereof, the method comprising: (a) detecting a KRas G12D mutation or a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a leukemia in a subject in need thereof, the method comprising: (a) detecting a KRas G12D mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a leukemia in a subject in need thereof, the method comprising: (a) detecting a KRas G12R mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a leukemia in a subject in need thereof, the method comprising: (a) detecting a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein.

In some embodiments, the cancer is a melanoma. In some embodiments, the melanoma has a KRas dysregulation (e.g., a KRas mutation or amplification). For example, the melanoma has a KRas mutation selected from the group consisting of: a KRas G12C mutation, a KRas G12D mutation, a KRas G12R mutation, a KRas G13D mutation, a KRas G13V mutation, a KRas a KRas Q61K mutation, a KRas Q61L mutation, and a KRas Q61R mutation. In some embodiments, the melanoma has a KRas G12D mutation or a KRas G12R mutation. In some embodiments, the melanoma has a KRas G12D mutation. In some embodiments, the melanoma has a KRas G12R mutation.

Also provided herein is a method of treating a melanoma in a subject in need thereof, the method comprising: (a) detecting a KRas dysregulation (e.g., a KRas mutation or amplification) in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Also provided herein is a method of treating a melanoma in a subject in need thereof, the method comprising: (a) detecting a KRas G12C mutation, a KRas G12D mutation, a KRas G12R mutation, a KRas G13D mutation, a KRas G13V mutation, a KRas a KRas Q61K mutation, a KRas Q61L mutation, or a KRas Q61R mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a melanoma in a subject in need thereof, the method comprising: (a) detecting a KRas G12D mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a melanoma in a subject in need thereof, the method comprising: (a) detecting a KRas G12R mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein.

In some embodiments, the cancer is a lung cancer (e.g., non-small cell lung cancer). In some embodiments, the lung cancer (e.g., non-small cell lung cancer) has a KRas dysregulation (e.g., a KRas mutation or amplification). For example, the lung cancer (e.g., non-small cell lung cancer) has a KRas mutation selected from the group consisting of: a KRas G12A mutation, a KRas G12C mutation, a KRas G12D mutation, a KRas G12S mutation, a KRas G12V mutation, a KRas G13C mutation, a KRas G13D mutation, a KRas Q61H mutation, and a KRas Q61L mutation. In some embodiments, the lung cancer has a KRas G12D mutation or a KRas G12V mutation. In some embodiments, the lung cancer has a KRas G12D mutation. In some embodiments, the lung cancer has a KRas G12V mutation.

Also provided herein is a method of treating a lung cancer (e.g., non-small cell lung cancer) in a subject in need thereof, the method comprising: (a) detecting a KRas dysregulation (e.g., a KRas mutation or amplification) in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Also provided herein is a method of treating a lung cancer (e.g., non-small cell lung cancer) in a subject in need thereof, the method comprising: (a) detecting a KRas G12A mutation, a KRas G12C mutation, a KRas G12D mutation, a KRas G12S mutation, a KRas G12V mutation, a KRas G13C mutation, a KRas G13D mutation, a KRas Q61H mutation, or a KRas Q61L mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a lung cancer in a subject in need thereof, the method comprising: (a) detecting a KRas G12D mutation or a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a lung cancer in a subject in need thereof, the method comprising: (a) detecting a KRas G12D mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a lung cancer in a subject in need thereof, the method comprising: (a) detecting a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein.

In some embodiments, the cancer is a pancreatic cancer. In some embodiments, the pancreatic cancer has a KRas dysregulation (e.g., a KRas mutation or amplification). For example, the pancreatic cancer has a KRas mutation selected from the group consisting of: a KRas G12A mutation, a KRas G12C mutation, a KRas G12D mutation, a KRas G12R mutation, a KRas G12S mutation, a KRas G12V mutation, a KRas G13C mutation, a KRas Q61H mutation, and a KRas Q61R mutation. In some embodiments, the pancreatic cancer has a KRas G12D mutation, a KRas G12R mutation, or a KRas G12V mutation. In some embodiments, the pancreatic cancer has a KRas G12D mutation or a KRas G12V mutation. In some embodiments, the pancreatic cancer has a KRas G12D mutation. In some embodiments, the pancreatic cancer has a KRas G12R mutation. In some embodiments, the pancreatic cancer has a KRas G12V mutation.

Also provided herein is a method of treating a pancreatic cancer in a subject in need thereof, the method comprising: (a) detecting a KRas dysregulation (e.g., a KRas mutation or amplification) in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Also provided herein is a method of treating a pancreatic cancer in a subject in need thereof, the method comprising: (a) detecting a KRas G12A mutation, a KRas G12C mutation, a KRas G12D mutation, a KRas G12R mutation, a KRas G12S mutation, a KRas G12V mutation, a KRas G13C mutation, a KRas Q61H mutation, or a KRas Q61R mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a pancreatic cancer in a subject in need thereof, the method comprising: (a) detecting a KRas G12D mutation, a KRas G12R mutation, or a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a pancreatic cancer in a subject in need thereof, the method comprising: (a) detecting a KRas G12D mutation or a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a pancreatic cancer in a subject in need thereof, the method comprising: (a) detecting a KRas G12D mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a pancreatic cancer in a subject in need thereof, the method comprising: (a) detecting a KRas G12R mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a pancreatic cancer in a subject in need thereof, the method comprising: (a) detecting a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein.

In some embodiments, the cancer is a testicular cancer (e.g., seminoma). In some embodiments, the testicular cancer (e.g., seminoma) has a KRas dysregulation (e.g., a KRas mutation or amplification). For example, the testicular cancer (e.g., seminoma) has a KRas mutation selected from the group consisting of: a KRas G12A mutation, a KRas G12R mutation, a KRas G12S mutation, a KRas G12V mutation, a KRas Q61L mutation, a KRas Q61P mutation, and a KRas Q61R mutation. In some embodiments, the testicular cancer (e.g., seminoma) cancer has a KRas G12R mutation or a KRas G12V mutation. In some embodiments, the testicular cancer (e.g., seminoma) cancer has a KRas G12R mutation. In some embodiments, the testicular cancer (e.g., seminoma) cancer has a KRas G12V mutation.

Also provided herein is a method of treating a testicular cancer (e.g., seminoma) in a subject in need thereof, the method comprising: (a) detecting a KRas dysregulation (e.g., a KRas mutation or amplification) in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Also provided herein is a method of treating a testicular cancer (e.g., seminoma) in a subject in need thereof, the method comprising: (a) detecting a KRas G12A mutation, a KRas G12R mutation, a KRas G12S mutation, a KRas G12V mutation, a KRas Q61L mutation, a KRas Q61P mutation, or a KRas Q61R mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a testicular cancer (e.g., seminoma) in a subject in need thereof, the method comprising: (a) detecting a KRas G12R mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. Additionally provided herein is a method of treating a testicular cancer (e.g., seminoma) in a subject in need thereof, the method comprising: (a) detecting a KRas G12V mutation in a sample from the subject, and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein.

Also provided herein is a method of treating a bladder cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some embodiments, the method comprises determining that the bladder cancer has a KRas mutation selected from the group consisting of: a KRas G12C mutation, a KRas G12D mutation, a KRas G12R mutation, a KRas G12V mutation, a KRas G13D mutation, and a KRas Q61H mutation. In some such embodiments, the cancer is a KRas G12C-associated cancer, a KRas G12D-associated cancer, a KRas G12R-associated cancer, a KRas G12V-associated cancer, a KRas G13D-associated cancer, or a KRas Q61H-associated cancer. In some aspects of this embodiment, the cancer is a KRas G12D-associated cancer, a KRas G12R-associated cancer, or a KRas G12V-associated cancer. In some embodiments, the cancer is a KRas G12D-associated cancer. In some embodiments, the cancer is a KRas G12R-associated cancer. In some embodiments, the cancer is a KRas G12V-associated cancer. In some embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12D inhibitor, a KRas G12R inhibitor, a KRas G12V inhibitor, a KRas G13D inhibitor, a KRas Q61H inhibitor, or two or more thereof. In some aspects of this embodiment, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12R inhibitor, and/or a KRas G12V inhibitor. In some aspects of this embodiment, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12V inhibitor, or both.

Also provided herein is a method of treating a cervical cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some embodiments, the method comprises determining that the cervical cancer has a KRas mutation selected from the group consisting of: a KRas G12C mutation, a KRas G12D mutation, a KRas G12V mutation, and a KRas G13D mutation. In some embodiments, the cancer is a KRas G12C-associated cancer, a KRas G12D-associated cancer, a KRas G12V-associated cancer, or a KRas G13D-associated cancer. In some embodiments, the cancer is a KRas G12D-associated cancer or a KRas G12V-associated cancer. In some embodiments, the cancer is a KRas G12D-associated cancer. In some embodiments, the cancer is a KRas G12V-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12D inhibitor, a KRas G12V inhibitor, a KRas G13D inhibitor, or two or more thereof. In some aspects of this embodiment, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12V inhibitor, or both.

Also provided herein is a method of treating a colorectal cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some embodiments, the method comprises determining that the colorectal cancer has a KRas mutation selected from the group consisting of: a KRas G12A mutation, a KRas G12C mutation, a KRas G12D mutation, a KRas G12R mutation, a KRas G12S mutation, a KRas G12V mutation, a KRas G13C mutation, a KRas G13D mutation, a KRas G13V mutation, a KRas Q61E mutation, a KRas Q61H mutation, a KRas Q61K mutation, a KRas Q61L mutation, a KRas Q61P mutation, and a KRas Q61R mutation. In some embodiments, the cancer is a KRas G12A-associated cancer, a KRas G12C-associated cancer, a KRas G12D-associated cancer, a KRas G12R-associated cancer, a KRas G12S-associated cancer, a KRas G12V-associated cancer, a KRas G13C-associated cancer, a KRas G13D-associated cancer, a KRas G13V-associated cancer, a KRas Q61E-associated cancer, a KRas Q61H-associated cancer, a KRas Q61K-associated cancer, a KRas Q61L-associated cancer, a KRas Q61P-associated cancer, or a KRas Q61R-associated cancer. In some aspects of this embodiment, the cancer is a KRas G12D-associated cancer, a KRas G12R-associated cancer, or a KRas G12V-associated cancer. In some embodiments, the cancer is a KRas G12D-associated cancer or a KRas G12V-associated cancer. In some embodiments, the cancer is a KRas G12D-associated cancer. In some embodiments, the cancer is a KRas G12R-associated cancer. In some embodiments, the cancer is a KRas G12V-associated cancer. In some embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12C inhibitor, a KRas G12D inhibitor, a KRas G12R inhibitor, a KRas G12S inhibitor, a KRas G12V inhibitor, a KRas G13C inhibitor, a KRas G13D inhibitor, a KRas G13V inhibitor, a KRas Q61E inhibitor, a KRas Q61H inhibitor, a KRas Q61K inhibitor, a KRas Q61L inhibitor, a KRas Q61P inhibitor, a KRas Q61R inhibitor, or two or more thereof. In some aspects of this embodiment, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12R inhibitor, and/or a KRas G12V inhibitor. In some aspects of this embodiment, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12V inhibitor, or both.

Also provided herein is a method of treating an endometrial cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some embodiments, the method comprises determining that the endometrial cancer has a KRas mutation selected from the group consisting of: a KRas G12A mutation, a KRas G12C mutation, a KRas G12D mutation, a KRas G12S mutation, a KRas G12V mutation, a KRas G13C mutation, a KRas G13D mutation, a KRas G13V mutation, a KRas Q61H mutation, and a KRas Q61L mutation. In some embodiments, the cancer is a KRas G12A-associated cancer, a KRas G12C-associated cancer, a KRas G12D-associated cancer, a KRas G12S-associated cancer, a KRas G12V-associated cancer, a KRas G13C-associated cancer, a KRas G13D-associated cancer, a KRas G13V-associated cancer, a KRas Q61H-associated cancer, or a KRas Q61L-associated cancer. In some embodiments, the cancer is a KRas G12D-associated cancer or a KRas G12V-associated cancer. In some embodiments, the cancer is a KRas G12D-associated cancer. In some embodiments, the cancer is a KRas G12V-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12C inhibitor, a KRas G12D inhibitor, a KRas G12S inhibitor, a KRas G12V inhibitor, a KRas G13C inhibitor, a KRas G13D inhibitor, a KRas G13V inhibitor, a KRas Q61H inhibitor, a KRas Q61L inhibitor, or two or more thereof. In some aspects of this embodiment, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12V inhibitor, or both.

Also provided herein is a method of treating an esophageal or stomach cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some embodiments, the method comprises determining that the esophageal or stomach cancer has a KRas mutation selected from the group consisting of: a KRas G12C mutation, a KRas G12D mutation, a KRas G12S mutation, a KRas G12V mutation, a KRas G13C mutation, a KRas G13D mutation, and a KRas Q61H mutation. In some embodiments, the cancer is a KRas G12C-associated cancer, a KRas G12D-associated cancer, a KRas G12S-associated cancer, a KRas G12V-associated cancer, a KRas G13C-associated cancer, a KRas G13D-associated cancer, or a KRas Q61H-associated cancer. In some embodiments, the cancer is a KRas G12D-associated cancer or a KRas G12V-associated cancer. In some embodiments, the cancer is a KRas G12D-associated cancer. In some embodiments, the cancer is a KRas G12V-associated cancer. In some embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12D inhibitor, a KRas G12S inhibitor, a KRas G12V inhibitor, a KRas G13C inhibitor, a KRas G13D inhibitor, a KRas Q61H inhibitor, or two or more thereof. In some aspects of this embodiment, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12V inhibitor, or both.

Also provided herein is a method of treating a leukemia in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some embodiments, the method comprises determining that the leukemia has a KRas mutation selected from the group consisting of: a KRas G12A mutation, a KRas G12C mutation, a KRas G12D mutation, a KRas G12R mutation, a KRas G12S mutation, a KRas G12V mutation, a KRas G13C mutation, a KRas G13D mutation, a KRas G13V mutation, a KRas Q61E mutation, a KRas Q61H mutation, a KRas Q61K mutation, a KRas Q61L mutation, a KRas Q61P mutation, and a KRas Q61R mutation. In some embodiments, the cancer is a KRas G12A-associated cancer, a KRas G12C-associated cancer, a KRas G12D-associated cancer, a KRas G12R-associated cancer, a KRas G12S-associated cancer, a KRas G12V-associated cancer, a KRas G13C-associated cancer, a KRas G13D-associated cancer, a KRas G13V-associated cancer, a KRas Q61E-associated cancer, a KRas Q61H-associated cancer, a KRas Q61K-associated cancer, a KRas Q61L-associated cancer, a KRas Q61P-associated cancer, or a KRas Q61R-associated cancer. In some aspects of this embodiment, the cancer is a KRas G12D-associated cancer, a KRas G12R-associated cancer, or a KRas G12V-associated cancer. In some embodiments, the cancer is a KRas G12D-associated cancer or a KRas G12V-associated cancer. In some embodiments, the cancer is a KRas G12D-associated cancer. In some embodiments, the cancer is a KRas G12R-associated cancer. In some embodiments, the cancer is a KRas G12V-associated cancer. In some embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12C inhibitor, a KRas G12D inhibitor, a KRas G12R inhibitor, a KRas G12S inhibitor, a KRas G12V inhibitor, a KRas G13C inhibitor, a KRas G13D inhibitor, a KRas G13V inhibitor, a KRas Q61E inhibitor, a KRas Q61H inhibitor, a KRas Q61K inhibitor, a KRas Q61L inhibitor, a KRas Q61P inhibitor, a KRas Q61R inhibitor, or two or more thereof. In some aspects of this embodiment, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12R inhibitor, and/or a KRas G12V inhibitor. In some aspects of this embodiment, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12V inhibitor, or both.

Also provided herein is a method of treating a melanoma in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some embodiments, the method comprises determining that the melanoma has a KRas mutation selected from the group consisting of: a KRas G12C mutation, a KRas G12D mutation, a KRas G12R mutation, a KRas G13D mutation, a KRas G13V mutation, a KRas Q61K mutation, a KRas Q61L mutation, and a KRas Q61R mutation. In some embodiments, the cancer is a KRas G12C-associated cancer, a KRas G12D-associated cancer, a KRas G12R-associated cancer, a KRas G13D-associated cancer, a KRas G13V-associated cancer, a KRas Q61K-associated cancer, a KRas Q61L-associated cancer, or a KRas Q61R-associated cancer. In some embodiments, the cancer is a KRas G12D-associated cancer or a KRas G12R-associated cancer. In some embodiments, the cancer is a KRas G12D-associated cancer. In some embodiments, the cancer is a KRas G12R-associated cancer. In some embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12D inhibitor, a KRas G12R inhibitor, a KRas G13D inhibitor, a KRas G13V inhibitor, a KRas Q61K inhibitor, a KRas Q61L inhibitor, a KRas Q61R inhibitor, or two or more thereof. In some aspects of this embodiment, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12R inhibitor, or both.

Also provided herein is a method of treating a lung cancer (e.g., NSCLC) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some embodiments, the method comprises determining that the lung cancer (e.g., NSCLC) has a KRas mutation selected from the group consisting of: KRas G12A mutation, a KRas G12C mutation, a KRas G12D mutation, a KRas G12S mutation, a KRas G12V mutation, a KRas G13C-associated cancer, a KRas G13D mutation, a KRas Q61H mutation, and a KRas Q61L mutation. In some embodiments, the cancer is a KRas G12A-associated cancer, a KRas G12C-associated cancer, a KRas G12D-associated cancer, a KRas G12S-associated cancer, a KRas G12V-associated cancer, a KRas G13C-associated cancer, a KRas G13D-associated cancer, a KRas Q61H-associated cancer, or a KRas Q61L-associated cancer. In some embodiments, the cancer is a KRas G12D-associated cancer or a KRas G12V-associated cancer. In some embodiments, the cancer is a KRas G12D-associated cancer. In some embodiments, the cancer is a KRas G12V-associated cancer. In some embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12C inhibitor, a KRas G12D inhibitor, a KRas G12S inhibitor, a KRas G12V inhibitor, a KRas G13C-associated cancer, a KRas G13D inhibitor, a KRas Q61H inhibitor, a KRas Q61L inhibitor, or two or more thereof. In some aspects of this embodiment, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, a KRas G12D inhibitor, a KRas G12V inhibitor, or both.

Also provided herein is a method of treating a pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some embodiments, the method comprises determining that the pancreatic cancer has a KRas mutation selected from the group consisting of: a KRas G12A mutation, a KRas G12C mutation, a KRas G12D mutation, a KRas G12R mutation, a KRas G12S mutation, a KRas G12V mutation, a KRas G13C-associated cancer, and a KRas Q61H mutation. In some embodiments, the cancer is a KRas G12A-associated cancer, a KRas G12C-associated cancer, a KRas G12D-associated cancer, a KRas G12R-associated cancer, a KRas G12S-associated cancer, a KRas G12V-associated cancer, a KRas G13C-associated cancer, or a KRas Q61H-associated cancer. In some aspects of this embodiment, the cancer is a KRas G12D-associated cancer, a KRas G12R-associated cancer, or a KRas G12V-associated cancer. In some embodiments, the cancer is a KRas G12D-associated cancer or a KRas G12V-associated cancer. In some embodiments, the cancer is a KRas G12D-associated cancer. In some embodiments, the cancer is a KRas G12R-associated cancer. In some embodiments, the cancer is a KRas G12V-associated cancer. In some embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12C inhibitor, a KRas G12D inhibitor, a KRas G12R inhibitor, a KRas G12S inhibitor, a KRas G12V inhibitor, a KRas G13C inhibitor, a KRas Q61H inhibitor, or two or more thereof. In some aspects of this embodiment, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12R inhibitor, and/or a KRas G12V inhibitor. In some aspects of this embodiment, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12V inhibitor, or both.

Also provided herein is a method of treating a testicular cancer (e.g., seminoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some embodiments, the method comprises determining that the testicular cancer (e.g., seminoma) has a KRas mutation selected from the group consisting of: a KRas G12A mutation, a KRas G12R mutation, a KRas G12S mutation, a KRas G12V mutation, a KRas Q61L mutation, a KRas Q61P mutation, and a KRas Q61R mutation. In some embodiments, the cancer is a KRas G12A-associated cancer, a KRas G12R-associated cancer, a KRas G12S-associated cancer, a KRas G12V-associated cancer, a KRas Q61L-associated cancer, a KRas Q61P-associated cancer, or a KRas Q61R-associated cancer. In some aspects of this embodiment, the cancer is a KRas G12R-associated cancer or a KRas G12V-associated cancer. In some embodiments, the cancer is a KRas G12R-associated cancer. In some embodiments, the cancer is a KRas G12V-associated cancer. In some embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12R inhibitor, a KRas G12S inhibitor, a KRas G12V inhibitor, a KRas Q61L inhibitor, a KRas Q61P inhibitor, a KRas Q61R inhibitor, or two or more thereof. In some aspects of this embodiment, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas G12V inhibitor, or both.

In some embodiments, the KRas-associated cancer is bladder cancer, breast cancer, cervical cancer, colorectal cancer, endometrial cancer, esophageal or stomach cancer, liver cancer, skin cancer (e.g., melanoma), lung cancer (e.g., NSCLC), or pancreatic cancer. In some embodiments, the cancer has a KRas G12C mutation or a KRas G12D mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12D inhibitor, or both.

Also provided herein is a method of treating a KRas G12C-associated cancer or a KRas G12D-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bladder cancer, breast cancer, cervical cancer, colorectal cancer, endometrial cancer, esophageal or stomach cancer, liver cancer, skin cancer (e.g., melanoma), lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12D inhibitor, or both.

Also provided herein is a method of treating bladder cancer, breast cancer, cervical cancer, colorectal cancer, endometrial cancer, esophageal or stomach cancer, liver cancer, skin cancer (e.g., melanoma), lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12C-associated cancer or a KRas G12D-associated cancer, or both. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12D inhibitor, or both.

In some embodiments, the KRas-associated cancer is bladder cancer, breast cancer, cervical cancer, colorectal cancer, endometrial cancer, esophageal or stomach cancer, leukemia, lung cancer (e.g., NSCLC), pancreatic cancer, or kidney cancer. In some embodiments, the cancer has a KRas G12D mutation or a KRas G12V mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12V inhibitor, or both.

Also provided herein is a method of treating a KRas G12D-associated cancer or a KRas G12V-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bladder cancer, breast cancer, cervical cancer, colorectal cancer, endometrial cancer, esophageal or stomach cancer, leukemia, lung cancer (e.g., NSCLC), pancreatic cancer, or kidney cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12V inhibitor, or both.

Also provided herein is a method of treating bladder cancer, breast cancer, cervical cancer, colorectal cancer, endometrial cancer, esophageal or stomach cancer, leukemia, lung cancer (e.g., NSCLC), pancreatic cancer, or kidney cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12D-associated cancer or a KRas G12V-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12V inhibitor, or both.

In some embodiments, the KRas-associated cancer is bladder cancer, cervical cancer, colorectal cancer, endometrial cancer, esophageal or stomach cancer, liver cancer, leukemia, skin cancer (e.g., melanoma), or lung cancer (e.g., NSCLC). In some embodiments, the cancer has a KRas G12D mutation or a KRas G13D mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G13D inhibitor, or both.

Also provided herein is a method of treating a KRas G12D-associated cancer or a KRas G13D-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bladder cancer, cervical cancer, colorectal cancer, endometrial cancer, esophageal or stomach cancer, liver cancer, leukemia, skin cancer (e.g., melanoma), or lung cancer (e.g., NSCLC). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G13D inhibitor, or both.

Also provided herein is a method of treating bladder cancer, cervical cancer, colorectal cancer, endometrial cancer, esophageal or stomach cancer, liver cancer, leukemia, skin cancer (e.g., melanoma), or lung cancer (e.g., NSCLC) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12D-associated cancer or a KRas G13D-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G13D inhibitor, or both.

In some embodiments, the KRas-associated cancer is bladder cancer, breast cancer, cervical cancer, colorectal cancer, endometrial cancer, esophageal or stomach cancer, lung cancer (e.g., NSCLC), or pancreatic cancer. In some embodiments, the cancer has a KRas G12C mutation or a KRas G12V mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12V inhibitor, or both.

Also provided herein is a method of treating a KRas G12C-associated cancer or a KRas G12V-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bladder cancer, breast cancer, cervical cancer, colorectal cancer, endometrial cancer, esophageal or stomach cancer, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12V inhibitor, or both.

Also provided herein is a method of treating bladder cancer, breast cancer, cervical cancer, colorectal cancer, endometrial cancer, esophageal or stomach cancer, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12C-associated cancer or a KRas G12V-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12V inhibitor, or both.

In some embodiments, the KRas-associated cancer is bladder cancer, cervical cancer, colorectal cancer, endometrial cancer, esophageal or stomach cancer, liver cancer, skin cancer (e.g., melanoma), or lung cancer (e.g., NSCLC). In some embodiments, the cancer has a KRas G12C mutation or a KRas G13D mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G13D inhibitor, or both.

Also provided herein is a method of treating a KRas G12C-associated cancer or a KRas G13D-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bladder cancer, cervical cancer, colorectal cancer, endometrial cancer, esophageal or stomach cancer, liver cancer, skin cancer (e.g., melanoma), or lung cancer (e.g., NSCLC). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G13D inhibitor, or both.

Also provided herein is a method of treating bladder cancer, cervical cancer, colorectal cancer, endometrial cancer, esophageal or stomach cancer, liver cancer, skin cancer (e.g., melanoma), or lung cancer (e.g., NSCLC) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (TT-f), (II-g), or (II-h)), or Formula (Aa)), or a pharmaceutically acceptable salt thereof, or a pharmaceutical composition as described herein. In some such embodiments, the cancer is a KRas G12C-associated cancer or a KRas G13D-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G13D inhibitor, or both.

In some embodiments, the KRas-associated cancer is bladder cancer, colorectal cancer, endometrial cancer, esophageal or stomach cancer, leukemia, lung cancer (e.g., NSCLC), or pancreatic cancer. In some embodiments, the cancer has a KRas G12D mutation or a KRas Q61H mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas Q61H inhibitor, or both.

Also provided herein is a method of treating a KRas G12D-associated cancer or a KRas Q61H-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bladder cancer, colorectal cancer, endometrial cancer, esophageal or stomach cancer, leukemia, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas Q61H inhibitor, or both.

Also provided herein is a method of treating bladder cancer, colorectal cancer, endometrial cancer, esophageal or stomach cancer, leukemia, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12D-associated cancer or a KRas Q61H-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas Q61H inhibitor, or both.

In some embodiments, the KRas-associated cancer is bladder cancer, cervical cancer, colorectal cancer, endometrial cancer, esophageal or stomach cancer, leukemia, or lung cancer (e.g., NSCLC). In some embodiments, the cancer has a KRas G12V mutation or a KRas G13D mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas G13D inhibitor, or both.

Also provided herein is a method of treating a KRas G12V-associated cancer or a KRas G13D-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bladder cancer, cervical cancer, colorectal cancer, endometrial cancer, esophageal or stomach cancer, leukemia, or lung cancer (e.g., NSCLC). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas G13D inhibitor, or both.

Also provided herein is a method of treating bladder cancer, cervical cancer, colorectal cancer, endometrial cancer, esophageal or stomach cancer, leukemia, or lung cancer (e.g., NSCLC) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12V-associated cancer or a KRas G13V-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas G13D inhibitor, or both.

In some embodiments, the KRas-associated cancer is bladder cancer, colorectal cancer, endometrial cancer, esophageal or stomach cancer, leukemia, lung cancer (e.g., NSCLC), or pancreatic cancer. In some embodiments, the cancer has a KRas G12V mutation or a KRas Q61H mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas Q61H inhibitor, or both.

Also provided herein is a method of treating a KRas G12V-associated cancer or a KRas Q61H-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bladder cancer, colorectal cancer, endometrial cancer, esophageal or stomach cancer, leukemia, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas Q61H inhibitor, or both.

Also provided herein is a method of treating bladder cancer, colorectal cancer, endometrial cancer, esophageal or stomach cancer, leukemia, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12V-associated cancer or a KRas Q61H-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas Q61H inhibitor, or both.

In some embodiments, the KRas-associated cancer is bladder cancer, colorectal cancer, endometrial cancer, esophageal or stomach cancer, lung cancer (e.g., NSCLC), or pancreatic cancer. In some embodiments, the cancer has a KRas G12C mutation or a KRas Q61H mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas Q61H inhibitor, or both.

Also provided herein is a method of treating a KRas G12C-associated cancer or a KRas Q61H-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bladder cancer, colorectal cancer, endometrial cancer, esophageal or stomach cancer, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas Q61H inhibitor, or both.

Also provided herein is a method of treating bladder cancer, colorectal cancer, endometrial cancer, esophageal or stomach cancer, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12C-associated cancer or a KRas Q61H-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas Q61H inhibitor, or both.

In some embodiments, the KRas-associated cancer is colorectal cancer, endometrial cancer, esophageal or stomach cancer, lung cancer (e.g., NSCLC), pancreatic cancer, or testicular cancer (e.g., seminoma). In some embodiments, the cancer has a KRas G12S mutation or a KRas G12V mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas G12V inhibitor, or both.

Also provided herein is a method of treating a KRas G12S-associated cancer or a KRas G12V-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, esophageal or stomach cancer, lung cancer (e.g., NSCLC), pancreatic cancer, or testicular cancer (e.g., seminoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas G12V inhibitor, or both.

Also provided herein is a method of treating colorectal cancer, endometrial cancer, esophageal or stomach cancer, lung cancer (e.g., NSCLC), pancreatic cancer, or testicular cancer (e.g., seminoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12S-associated cancer or a KRas G12V-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas G12V inhibitor, or both.

In some embodiments, the KRas-associated cancer is colorectal cancer, endometrial cancer, lung cancer (e.g., NSCLC), pancreatic cancer, or testicular cancer (e.g., seminoma). In some embodiments, the cancer has a KRas G12A mutation or a KRas G12S mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12S inhibitor, or both.

Also provided herein is a method of treating a KRas G12A-associated cancer or a KRas G12S-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, lung cancer (e.g., NSCLC), pancreatic cancer, or testicular cancer (e.g., seminoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12S inhibitor, or both.

Also provided herein is a method of treating colorectal cancer, endometrial cancer, lung cancer (e.g., NSCLC), pancreatic cancer, or testicular cancer (e.g., seminoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12A-associated cancer or a KRas G12S-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12S inhibitor, or both.

In some embodiments, the KRas-associated cancer is colorectal cancer, endometrial cancer, lung cancer (e.g., NSCLC), pancreatic cancer, or testicular cancer (e.g., seminoma). In some embodiments, the cancer has a KRas G12A mutation or a KRas G12V mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12V inhibitor, or both.

Also provided herein is a method of treating a KRas G12A-associated cancer or a KRas G12V-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, lung cancer (e.g., NSCLC), pancreatic cancer, or testicular cancer (e.g., seminoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12V inhibitor, or both.

Also provided herein is a method of treating colorectal cancer, endometrial cancer, lung cancer (e.g., NSCLC), pancreatic cancer, or testicular cancer (e.g., seminoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12A-associated cancer or a KRas G12V-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12V inhibitor, or both.

In some embodiments, the KRas-associated cancer is colorectal cancer, endometrial cancer, esophageal or stomach cancer, lung cancer (e.g., NSCLC), or pancreatic cancer. In some embodiments, the cancer has a KRas G12C mutation or a KRas G12S mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12S inhibitor, or both.

Also provided herein is a method of treating a KRas G12C-associated cancer or a KRas G12S-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, esophageal or stomach cancer, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12S inhibitor, or both.

Also provided herein is a method of treating colorectal cancer, endometrial cancer, esophageal or stomach cancer, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12C-associated cancer or a KRas G12S-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12S inhibitor, or both.

In some embodiments, the KRas-associated cancer is bladder cancer, colorectal cancer, skin cancer (e.g., melanoma), pancreatic cancer, or prostate cancer. In some embodiments, the cancer has a KRas G12D mutation or a KRas G12R mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12R inhibitor, or both.

Also provided herein is a method of treating a KRas G12D-associated cancer or a KRas G12R-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bladder cancer, colorectal cancer, skin cancer (e.g., melanoma), pancreatic cancer, or prostate cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12R inhibitor, or both.

Also provided herein is a method of treating bladder cancer, colorectal cancer, skin cancer (e.g., melanoma), pancreatic cancer, or prostate cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12D-associated cancer or a KRas G12R-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12R inhibitor, or both.

In some embodiments, the KRas-associated cancer is colorectal cancer, endometrial cancer, esophageal or stomach cancer, lung cancer (e.g., NSCLC), or pancreatic cancer. In some embodiments, the cancer has a KRas G12D mutation or a KRas G12S mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12S inhibitor, or both.

Also provided herein is a method of treating a KRas G12D-associated cancer or a KRas G12S-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, esophageal or stomach cancer, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12S inhibitor, or both.

Also provided herein is a method of treating colorectal cancer, endometrial cancer, esophageal or stomach cancer, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12D-associated cancer or a KRas G12S-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G12S inhibitor, or both.

In some embodiments, the KRas-associated cancer is bladder cancer, colorectal cancer, ovarian cancer, pancreatic cancer, or testicular cancer (e.g., seminoma). In some embodiments, the cancer has a KRas G12R mutation or a KRas G12V mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas G12V inhibitor, or both.

Also provided herein is a method of treating a KRas G12R-associated cancer or a KRas G12V-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bladder cancer, colorectal cancer, ovarian cancer, pancreatic cancer, or testicular cancer (e.g., seminoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas G12V inhibitor, or both.

Also provided herein is a method of treating bladder cancer, colorectal cancer, ovarian cancer, pancreatic cancer, or testicular cancer (e.g., seminoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12R-associated cancer or a KRas G12V-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas G12V inhibitor, or both.

In some embodiments, the KRas-associated cancer is colorectal cancer, endometrial cancer, esophageal or stomach cancer, lung cancer (e.g., NSCLC), or pancreatic cancer. In some embodiments, the cancer has a KRas G12S mutation or a KRas Q61H mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas Q61H inhibitor, or both.

Also provided herein is a method of treating a KRas G12S-associated cancer or a KRas Q61H-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, esophageal or stomach cancer, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas Q61H inhibitor, or both.

Also provided herein is a method of treating colorectal cancer, endometrial cancer, esophageal or stomach cancer, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12S-associated cancer or a KRas Q61H-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas Q61H inhibitor, or both.

In some embodiments, the KRas-associated cancer is colorectal cancer, endometrial cancer, lung cancer (e.g., NSCLC), ovarian cancer, or testicular cancer (e.g., seminoma). In some embodiments, the cancer has a KRas G12V mutation or a KRas Q61L mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas Q61L inhibitor, or both.

Also provided herein is a method of treating a KRas G12V-associated cancer or a KRas Q61L-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, lung cancer (e.g., NSCLC), ovarian cancer, or testicular cancer (e.g., seminoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas Q61L inhibitor, or both.

Also provided herein is a method of treating colorectal cancer, endometrial cancer, lung cancer (e.g., NSCLC), ovarian cancer, or testicular cancer (e.g., seminoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12V-associated cancer or a KRas Q61L-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas Q61L inhibitor, or both.

In some embodiments, the KRas-associated cancer is colorectal cancer, endometrial cancer, lung cancer (e.g., NSCLC), or pancreatic cancer. In some embodiments, the cancer has a KRas G12A mutation or a KRas G12C mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12C inhibitor, or both.

Also provided herein is a method of treating a KRas G12A-associated cancer or a KRas G12C-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12C inhibitor, or both.

Also provided herein is a method of treating colorectal cancer, endometrial cancer, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12A-associated cancer or a KRas G12C-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12C inhibitor, or both.

In some embodiments, the KRas-associated cancer is colorectal cancer, endometrial cancer, lung cancer (e.g., NSCLC), or pancreatic cancer. In some embodiments, the cancer has a KRas G12A mutation or a KRas G12D mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12D inhibitor, or both.

Also provided herein is a method of treating a KRas G12A-associated cancer or a KRas G12D-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12D inhibitor, or both.

Also provided herein is a method of treating colorectal cancer, endometrial cancer, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12A-associated cancer or a KRas G12D-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12D inhibitor, or both.

In some embodiments, the KRas-associated cancer is colorectal cancer, endometrial cancer, lung cancer (e.g., NSCLC), or pancreatic cancer. In some embodiments, the cancer has a KRas G12A mutation or a KRas G13C mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G13C inhibitor, or both.

Also provided herein is a method of treating a KRas G12A-associated cancer or a KRas G13C-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G13C inhibitor, or both.

Also provided herein is a method of treating colorectal cancer, endometrial cancer, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12A-associated cancer or a KRas G13C-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G13C inhibitor, or both.

In some embodiments, the KRas-associated cancer is colorectal cancer, endometrial cancer, lung cancer (e.g., NSCLC), or pancreatic cancer. In some embodiments, the cancer has a KRas G12A mutation or a KRas Q61H mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas Q61H inhibitor, or both.

Also provided herein is a method of treating a KRas G12A-associated cancer or a KRas Q61H-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas Q61H inhibitor, or both.

Also provided herein is a method of treating colorectal cancer, endometrial cancer, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12A-associated cancer or a KRas Q61H-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas Q61H inhibitor, or both.

In some embodiments, the KRas-associated cancer is colorectal cancer, endometrial cancer, lung cancer (e.g., NSCLC), or testicular cancer (e.g., seminoma). In some embodiments, the cancer has a KRas G12A mutation or a KRas Q61L mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas Q61L inhibitor, or both.

Also provided herein is a method of treating a KRas G12A-associated cancer or a KRas Q61L-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, lung cancer (e.g., NSCLC), or testicular cancer (e.g., seminoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas Q61L inhibitor, or both.

Also provided herein is a method of treating colorectal cancer, endometrial cancer, lung cancer (e.g., NSCLC), or testicular cancer (e.g., seminoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12A-associated cancer or a KRas Q61L-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas Q61L inhibitor, or both.

In some embodiments, the KRas-associated cancer is bladder cancer, colorectal cancer, skin cancer (e.g., melanoma), or pancreatic cancer. In some embodiments, the cancer has a KRas G12C mutation or a KRas G12R mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salt thereof, is a KRas G12C inhibitor, a KRas G12R inhibitor, or both.

Also provided herein is a method of treating a KRas G12C-associated cancer or a KRas G12R-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bladder cancer, colorectal cancer, skin cancer (e.g., melanoma), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12R inhibitor, or both.

Also provided herein is a method of treating bladder cancer, colorectal cancer, skin cancer (e.g., melanoma), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12C-associated cancer or a KRas G12R-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G12R inhibitor, or both.

In some embodiments, the KRas-associated cancer is colorectal cancer, endometrial cancer, lung cancer (e.g., NSCLC), or pancreatic cancer. In some embodiments, the cancer has a KRas G12C mutation or a KRas G13C mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G13C inhibitor, or both.

Also provided herein is a method of treating a KRas G12C-associated cancer or a KRas G13C-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G13C inhibitor, or both.

Also provided herein is a method of treating colorectal cancer, endometrial cancer, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12C-associated cancer or a KRas G13C-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas G13C inhibitor, or both.

In some embodiments, the KRas-associated cancer is colorectal cancer, endometrial cancer, skin cancer (e.g., melanoma), or lung cancer (e.g., NSCLC). In some embodiments, the cancer has a KRas G12C mutation or a KRas Q61L mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas Q61L inhibitor, or both.

Also provided herein is a method of treating a KRas G12C-associated cancer or a KRas Q61L-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, skin cancer (e.g., melanoma), or lung cancer (e.g., NSCLC). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas Q61L inhibitor, or both.

Also provided herein is a method of treating colorectal cancer, endometrial cancer, skin cancer (e.g., melanoma), or lung cancer (e.g., NSCLC) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12C-associated cancer or a KRas Q61L-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas Q61L inhibitor, or both.

In some embodiments, the KRas-associated cancer is colorectal cancer, endometrial cancer, lung cancer (e.g., NSCLC), or pancreatic cancer. In some embodiments, the cancer has a KRas G12D mutation or a KRas G13C mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G13C inhibitor, or both.

Also provided herein is a method of treating a KRas G12D-associated cancer or a KRas G13C-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G13C inhibitor, or both.

Also provided herein is a method of treating colorectal cancer, endometrial cancer, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12D-associated cancer or a KRas G13C-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas G13C inhibitor, or both.

In some embodiments, the KRas-associated cancer is colorectal cancer, endometrial cancer, skin cancer (e.g., melanoma), or lung cancer (e.g., NSCLC). In some embodiments, the cancer has a KRas G12D mutation or a KRas Q61L mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas Q61L inhibitor, or both.

Also provided herein is a method of treating a KRas G12D-associated cancer or a KRas Q61L-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, skin cancer (e.g., melanoma), or lung cancer (e.g., NSCLC). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas Q61L inhibitor, or both.

Also provided herein is a method of treating colorectal cancer, endometrial cancer, skin cancer (e.g., melanoma), or lung cancer (e.g., NSCLC) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12D-associated cancer or a KRas Q61L-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas Q61L inhibitor, or both.

In some embodiments, the KRas-associated cancer is colorectal cancer, skin cancer (e.g., melanoma), ovarian cancer, or testicular cancer (e.g., seminoma). In some embodiments, the cancer has a KRas G12R mutation or a KRas Q61L mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas Q61L inhibitor, or both.

Also provided herein is a method of treating a KRas G12R-associated cancer or a KRas Q61L-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, skin cancer (e.g., melanoma), ovarian cancer, or testicular cancer (e.g., seminoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas Q61L inhibitor, or both.

Also provided herein is a method of treating colorectal cancer, skin cancer (e.g., melanoma), ovarian cancer, or testicular cancer (e.g., seminoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12R-associated cancer or a KRas Q61L-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas Q61L inhibitor, or both.

In some embodiments, the KRas-associated cancer is colorectal cancer, skin cancer (e.g., melanoma), pancreatic cancer, or testicular cancer (e.g., seminoma). In some embodiments, the cancer has a KRas G12R mutation or a KRas Q61R mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas Q61R inhibitor, or both.

Also provided herein is a method of treating a KRas G12R-associated cancer or a KRas Q61R-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, skin cancer (e.g., melanoma), pancreatic cancer, or testicular cancer (e.g., seminoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas Q61R inhibitor, or both.

Also provided herein is a method of treating colorectal cancer, skin cancer (e.g., melanoma), pancreatic cancer, or testicular cancer (e.g., seminoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12R-associated cancer or a KRas Q61R-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas Q61R inhibitor, or both.

In some embodiments, the KRas-associated cancer is colorectal cancer, endometrial cancer, lung cancer (e.g., NSCLC), or pancreatic cancer. In some embodiments, the cancer has a KRas G12S mutation or a KRas G13C mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas G13C inhibitor, or both.

Also provided herein is a method of treating a KRas G12S-associated cancer or a KRas G13C-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas G13C inhibitor, or both.

Also provided herein is a method of treating colorectal cancer, endometrial cancer, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12S-associated cancer or a KRas G13C-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas G13C inhibitor, or both.

In some embodiments, the KRas-associated cancer is colorectal cancer, endometrial cancer, esophageal or stomach cancer, or lung cancer (e.g., NSCLC). In some embodiments, the cancer has a KRas G12S mutation or a KRas G13D mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas G13D inhibitor, or both.

Also provided herein is a method of treating a KRas G12S-associated cancer or a KRas G13D-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, esophageal or stomach cancer, or lung cancer (e.g., NSCLC). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas G13D inhibitor, or both.

Also provided herein is a method of treating colorectal cancer, endometrial cancer, esophageal or stomach cancer, or lung cancer (e.g., NSCLC) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12S-associated cancer or a KRas G13D-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas G13D inhibitor, or both.

In some embodiments, the KRas-associated cancer is colorectal cancer, endometrial cancer, lung cancer (e.g., NSCLC), or testicular cancer (e.g., seminoma). In some embodiments, the cancer has a KRas G12S mutation or a KRas Q61L mutation. In some such embodiments, compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas Q61L inhibitor, or both.

Also provided herein is a method of treating a KRas G12S-associated cancer or a KRas Q61L-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, lung cancer (e.g., NSCLC), or testicular cancer (e.g., seminoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas Q61L inhibitor, or both.

Also provided herein is a method of treating colorectal cancer, endometrial cancer, lung cancer (e.g., NSCLC), or testicular cancer (e.g., seminoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12S-associated cancer or a KRas Q61L-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas Q61L inhibitor, or both.

In some embodiments, the KRas-associated cancer is colorectal cancer, endometrial cancer, lung cancer (e.g., NSCLC), or pancreatic cancer. In some embodiments, the cancer has a KRas G12V mutation or a KRas G13C mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas G13C inhibitor, or both.

Also provided herein is a method of treating a KRas G12V-associated cancer or a KRas G13C-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, lung cancer (e.g., NSCLC), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas G13C inhibitor, or both.

Also provided herein is a method of treating colorectal cancer, endometrial cancer, lung cancer (e.g., NSCLC), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12V-associated cancer or a KRas G13C-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas G13C inhibitor, or both.

In some embodiments, the KRas-associated cancer is colorectal cancer, pancreatic cancer, testicular cancer (e.g., seminoma), or thyroid cancer. In some embodiments, the cancer has a KRas G12V mutation or a KRas Q61R mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas Q61R inhibitor, or both.

Also provided herein is a method of treating a KRas G12V-associated cancer or a KRas Q61R-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, pancreatic cancer, testicular cancer (e.g., seminoma), or thyroid cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas Q61R inhibitor, or both.

Also provided herein is a method of treating colorectal cancer, pancreatic cancer, testicular cancer (e.g., seminoma), or thyroid cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12V-associated cancer or a KRas Q61R-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12V inhibitor, a KRas Q61R inhibitor, or both.

In some embodiments, the KRas-associated cancer is colorectal cancer, pancreatic cancer, or testicular cancer (e.g., seminoma). In some embodiments, the cancer has a KRas G12A mutation or a KRas G12R mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12R inhibitor, or both.

Also provided herein is a method of treating a KRas G12A-associated cancer or a KRas G12R-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, pancreatic cancer, or testicular cancer (e.g., seminoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12R inhibitor, or both.

Also provided herein is a method of treating colorectal cancer, pancreatic cancer, or testicular cancer (e.g., seminoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12A-associated cancer or a KRas G12R-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G12R inhibitor, or both.

In some embodiments, the KRas-associated cancer is colorectal cancer, endometrial cancer, or lung cancer (e.g., NSCLC). In some embodiments, the cancer has a KRas G12A mutation or a KRas G13D mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G13D inhibitor, or both.

Also provided herein is a method of treating a KRas G12A-associated cancer or a KRas G13D-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, endometrial cancer, or lung cancer (e.g., NSCLC). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G13D inhibitor, or both.

Also provided herein is a method of treating colorectal cancer, endometrial cancer, or lung cancer (e.g., NSCLC) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12A-associated cancer or a KRas G13D-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas G13D inhibitor, or both.

In some embodiments, the KRas-associated cancer is colorectal cancer, pancreatic cancer, or testicular cancer (e.g., seminoma). In some embodiments, the cancer has a KRas G12A mutation or a KRas Q61R mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (TT-f), (II-g), or (II-h)), or Formula (Aa)), or a pharmaceutically acceptable salt thereof, is a KRas G12A inhibitor, a KRas Q61R inhibitor, or both.

Also provided herein is a method of treating a KRas G12A-associated cancer or a KRas Q61R-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, pancreatic cancer, or testicular cancer (e.g., seminoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas Q61R inhibitor, or both.

Also provided herein is a method of treating colorectal cancer, pancreatic cancer, or testicular cancer (e.g., seminoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12A-associated cancer or a KRas Q61R-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12A inhibitor, a KRas Q61R inhibitor, or both.

In some embodiments, the KRas-associated cancer is colorectal cancer, skin cancer (e.g., melanoma), or pancreatic cancer. In some embodiments, the cancer has a KRas G12C mutation or a KRas Q61R mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas Q61R inhibitor, or both.

Also provided herein is a method of treating a KRas G12C-associated cancer or a KRas Q61R-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, skin cancer (e.g., melanoma), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas Q61R inhibitor, or both.

Also provided herein is a method of treating colorectal cancer, skin cancer (e.g., melanoma), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12C-associated cancer or a KRas Q61R-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12C inhibitor, a KRas Q61R inhibitor, or both.

In some embodiments, the KRas-associated cancer is colorectal cancer, skin cancer (e.g., melanoma), or pancreatic cancer. In some embodiments, the cancer has a KRas G12D mutation or a KRas Q61R mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas Q61R inhibitor, or both.

Also provided herein is a method of treating a KRas G12D-associated cancer or a KRas Q61R-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, skin cancer (e.g., melanoma), or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas Q61R inhibitor, or both.

Also provided herein is a method of treating colorectal cancer, skin cancer (e.g., melanoma), or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12D-associated cancer or a KRas Q61R-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12D inhibitor, a KRas Q61R inhibitor, or both.

In some embodiments, the KRas-associated cancer is colorectal cancer, pancreatic cancer, or testicular cancer (e.g., seminoma). In some embodiments, the cancer has a KRas G12R mutation or a KRas G12S mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas G12S inhibitor, or both.

Also provided herein is a method of treating a KRas G12R-associated cancer or a KRas G12S-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, pancreatic cancer, or testicular cancer (e.g., seminoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas G12S inhibitor, or both.

Also provided herein is a method of treating colorectal cancer, pancreatic cancer, or testicular cancer (e.g., seminoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12R-associated cancer or a KRas G12S-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas G12S inhibitor, or both.

In some embodiments, the KRas-associated cancer is bladder cancer, colorectal cancer, or skin cancer (e.g., melanoma). In some embodiments, the cancer has a KRas G12R mutation or a KRas G13D mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas G13D inhibitor, or both.

Also provided herein is a method of treating a KRas G12R-associated cancer or a KRas G13D-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bladder cancer, colorectal cancer, or skin cancer (e.g., melanoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas G13D inhibitor, or both.

Also provided herein is a method of treating bladder cancer, colorectal cancer, or skin cancer (e.g., melanoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12R-associated cancer or a KRas G13D-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas G13D inhibitor, or both.

In some embodiments, the KRas-associated cancer is bladder cancer, colorectal cancer, or pancreatic cancer. In some embodiments, the cancer has a KRas G12R mutation or a KRas Q61H mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas Q61H inhibitor, or both.

Also provided herein is a method of treating a KRas G12R-associated cancer or a KRas Q61H-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bladder cancer, colorectal cancer, or pancreatic cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas Q61H inhibitor, or both.

Also provided herein is a method of treating bladder cancer, colorectal cancer, or pancreatic cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12R-associated cancer or a KRas Q61H-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas Q61H inhibitor, or both.

In some embodiments, the KRas-associated cancer is bile duct cancer (e.g., cholangiocarcinoma), colorectal cancer, or skin cancer (e.g., melanoma). In some embodiments, the cancer has a KRas G12R mutation or a KRas Q61K mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas Q61K inhibitor, or both.

Also provided herein is a method of treating a KRas G12R-associated cancer or a KRas Q61K-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is bile duct cancer (e.g., cholangiocarcinoma), colorectal cancer, or skin cancer (e.g., melanoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas Q61K inhibitor, or both.

Also provided herein is a method of treating bile duct cancer (e.g., cholangiocarcinoma), colorectal cancer, or skin cancer (e.g., melanoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12R-associated cancer or a KRas Q61K-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12R inhibitor, a KRas Q61K inhibitor, or both.

In some embodiments, the KRas-associated cancer is colorectal cancer, pancreatic cancer, or testicular cancer (e.g., seminoma). In some embodiments, the cancer has a KRas G12S mutation or a KRas Q61R mutation. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas Q61R inhibitor, or both.

Also provided herein is a method of treating a KRas G12S-associated cancer or a KRas Q61R-associated cancer in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is colorectal cancer, pancreatic cancer, or testicular cancer (e.g., seminoma). In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas Q61R inhibitor, or both.

Also provided herein is a method of treating colorectal cancer, pancreatic cancer, or testicular cancer (e.g., seminoma) in a subject in need of such treatment, the method comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition as described herein. In some such embodiments, the cancer is a KRas G12S-associated cancer or a KRas Q61R-associated cancer. In some such embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is a KRas G12S inhibitor, a KRas Q61R inhibitor, or both.

Also provided herein is a method for treating a subject diagnosed with or identified as having a KRas-associated cancer, e.g., any of the exemplary mutant KRas-associated cancers disclosed herein, comprising administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)) or a pharmaceutically acceptable salt thereof, or a pharmaceutical composition thereof, as defined herein.

Accordingly, provided herein are methods for treating a subject diagnosed with (or identified as having) a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))) that include administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof. In some embodiments, the subject that has been identified or diagnosed as having a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))) through the use of a regulatory agency-approved, e.g., FDA-approved test or assay for identifying a dysregulation associated with KRas (e.g., a KRAS gene having a mutation corresponding to a mutation in a KRas protein and/or a KRas protein having a mutation, a KRAS gene copy number increase, and/or an increase in KRas mRNA or protein expression), in a subject or a biopsy sample from the subject or by performing any of the non-limiting examples of assays described herein. In some embodiments, the subject that has been identified or diagnosed as having a KRas-associated cancer through the use of a regulatory agency-approved, e.g., FDA-approved test or assay for identifying a KRas dysregulation (e.g., KRas mutation) in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))) in the subject; and (b) administering to the subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition thereof. Some embodiments of these methods further include administering to the subject another anticancer agent (e.g., a second compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, an immunotherapy, or any of the other anticancer agents described herein). In some embodiments, the subject was previously treated with another anticancer treatment, e.g., a kinase inhibitor (e.g., an EGFR inhibitor), at least partial resection of the tumor, radiation therapy, or a combination thereof. In some embodiments, the cancer in the subject is determined to have a KRas dysregulation (e.g., a KRas mutation (e.g., a KRas G12D mutation, a KRas G12R mutation, or a KRas G12V mutation (e.g., a KRas G12D mutation or a KRas G12V mutation))) through the use of a regulatory agency-approved, e.g., FDA-approved test or assay for identifying a KRas mutation (e.g., a KRAS gene having a mutation corresponding to a mutation in a KRas protein and/or a KRas protein having a mutation, a KRAS gene copy number increase, and/or an increase in KRas mRNA or protein expression), in a subject or a sample (e.g., a tumor sample or blood sample) from the subject or by performing any of the non-limiting examples of assays described herein. In some embodiments, the subject that has been identified or diagnosed as having a cancer with a KRas dysregulation (e.g., a KRas mutation (e.g., a KRas G12D mutation, a KRas G12R mutation, or a KRas G12V mutation (e.g., a KRas G12D mutation or a KRas G12V mutation))) through the use of a regulatory agency-approved, e.g., FDA-approved test or assay for identifying a KRas dysregulation in a subject or a biopsy sample from the subject or by performing any of the non-limiting examples of assays described herein. In some embodiments, the test or assay is provided as a kit.

Also provided are methods of treating a subject that include performing an assay on a sample (e.g., a tumor sample or a blood sample) obtained from the subject to determine whether the cancer in the subject has a KRas dysregulation (e.g., a KRas mutation (e.g., a KRas G12D mutation, a KRas G12R mutation, or a KRas G12V mutation (e.g., a KRas G12D mutation or a KRas G12V mutation))), and administering (e.g., specifically or selectively administering) a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition thereof, to the subject determined a cancer having a KRas dysregulation (e.g., a KRas mutation (e.g., a KRas G12D mutation, a KRas G12R mutation, or a KRas G12V mutation (e.g., a KRas G12D mutation or a KRas G12V mutation))). In some embodiments, provided are methods of treating a subject that include performing an assay on a sample (e.g., a tumor sample or a blood sample) obtained from the subject to determine whether the cancer in the subject has a KRas dysregulation (e.g., a KRAS gene having a mutation corresponding to a mutation in a KRas protein and/or a KRas protein having a mutation, a KRAS gene copy number increase, and/or an increase in KRas mRNA or protein expression), and administering (e.g., specifically or selectively administering) a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition thereof, to the subject having a cancer determined to have a KRas dysregulation. Some embodiments of these methods further include administering to the subject another anticancer agent (e.g., a second compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or immunotherapy). In some embodiments of these methods, the subject was previously treated with another anticancer treatment, e.g., a first KRas inhibitor, a kinase inhibitor (e.g., an EGFR inhibitor), at least partial resection of a tumor, radiation therapy, or a combination thereof. In some embodiments, the subject is a subject suspected of having a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))), a subject presenting with one or more symptoms of a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))), or a subject having an elevated risk of developing a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))). In some embodiments, the assay utilizes next generation sequencing, pyrosequencing, or immunohistochemistry. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art. Also provided is a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition thereof, for use in treating a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))) in a subject identified or diagnosed as having a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))) through a step of performing an assay (e.g., an in vitro assay) on a sample (e.g., a tumor sample or a blood sample) obtained from the subject to determine whether the cancer in the subject has a KRas dysregulation, where the presence of a KRas dysregulation (e.g., a KRAS gene having a mutation corresponding to a mutation in a KRas protein and/or a KRas protein having a mutation, a KRAS gene copy number increase, and/or an increase in KRas mRNA or protein expression), identifies that the cancer in the subject has a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))).

Also provided is the use of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, for the manufacture of a medicament for treating a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))) in a subject identified or diagnosed as having a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))) through a step of performing an assay on a sample obtained from the subject to determine whether the cancer in the subject has a KRas dysregulation (e.g., a KRAS gene having a mutation corresponding to a mutation in a KRas protein and/or a KRas protein having a mutation, a KRAS gene copy number increase, and/or an increase in KRas mRNA or protein expression) where the presence of a KRas dysregulation (e.g., a KRAS gene having a mutation corresponding to a mutation in a KRas protein and/or a KRas protein having a mutation, a KRAS gene copy number increase, and/or an increase in KRas mRNA or protein expression), identifies that the subject has a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))).

Some embodiments of any of the methods or uses described herein further include recording in the subject's clinical record (e.g., a computer readable medium) that the subject is determined to have a KRas dysregulation (e.g., a KRAS gene having a mutation corresponding to a mutation in a KRas protein and/or a KRas protein having a mutation, a KRAS gene copy number increase, and/or an increase in KRas mRNA or protein expression), through the performance of the assay, should be administered a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition thereof. In some embodiments, the assay utilizes next generation sequencing, pyrosequencing, or immunohistochemistry. In some embodiments, the assay is a regulatory agency-approved assay, e.g., FDA-approved kit. In some embodiments, the assay is a liquid biopsy.

Also provided is a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, for use in the treatment of a cancer in a subject in need thereof, or a subject identified or diagnosed as having a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))) (e.g., any of the KRas-associated cancers described herein). Also provided is the use of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, for the manufacture of a medicament for treating a cancer in a subject identified or diagnosed as having a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))) (e.g., any of the KRas-associated cancers described herein). In some embodiments, a mutant KRas-associated cancer is a cancer that was previously identified as having no KRas mutation (e.g., KRas wild type), for example, in a cancer that was previously identified as having no KRas mutation and then, later, a KRas mutation (e.g., a resistance mutation) was identified. In some embodiments, a subject is identified or diagnosed as having a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))) through the use of a regulatory agency-approved, e.g., FDA-approved, kit for identifying a KRas dysregulation, in a subject or a biopsy sample from the subject. As provided herein, a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))) includes those described herein and known in the art.

In some embodiments of any of the methods or uses described herein, the subject has been identified or diagnosed as having a cancer with a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification). In some embodiments of any of the methods or uses described herein, the subject has a cancer (e.g., a tumor sample) that is positive for a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification). In some embodiments of any of the methods or uses described herein, the subject can be a subject with a cancer (e.g., one or more tumor samples) that is positive for a KRas dysregulation (e.g., KRas mutation (e.g., a KRas G12D mutation, a KRas G12R mutation, or a KRas G12V mutation (e.g., a KRas G12D mutation or a KRas G12V mutation))). In some embodiments of any of the methods or uses described herein, the subject is suspected of having a mutant KRas-associated cancer (e.g., a cancer that was previously identified a cancer having no KRas mutation (e.g., KRas wild type)). In some embodiments, provided herein are methods for treating a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))) in a subject in need of such treatment, the method comprising a) detecting a KRas dysregulation (e.g., a KRAS gene having a mutation corresponding to a mutation in a KRas protein and/or a KRas protein having a mutation, a KRAS gene copy number increase, and/or an increase in KRas mRNA or protein expression) in a sample from the subject; and b) administering a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof.

In some embodiments, a mutant KRas-associated cancer is characterized by a mutation that arises from treatment with a first KRas inhibitor; for example, a mutant KRas-associated cancer as described herein can include one or more KRas mutations that confer resistance to treatment with a first KRas inhibitor. For example, a subject can acquire one or more of the following KRas mutations as a resistance mutation to a KRas G12C inhibitor: G12D, G12R, G12V, G12W, G13D, Q61H, R68S, H95D, H95Q, H95R, or Y96C. See, e.g., Awad et al. N Engl J Med. 2021 Jun. 24; 384(25):2382-2393.

As used herein, a “first inhibitor of KRas” or “first KRas inhibitor” is a KRas inhibitor as defined herein, but which does not include a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, as defined herein. As used herein, a “second inhibitor of KRas” or a “second KRas inhibitor” is a KRas inhibitor as defined herein, but which does not include a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof. When both a first and a second inhibitor of KRas are present in a method provided herein, the first and second inhibitors of KRas are different. In some embodiments, the first and/or second inhibitor of KRas bind in a different location than a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof.

Exemplary first and second inhibitors of KRas are described herein. In some embodiments, a first or a second inhibitor of KRas can be a KRas G12C inhibitor. In some embodiments, a first or second inhibitor of KRas can be selected from the group consisting of sotorasib, adragrasib, ARS-853, ARS-1620, ARS-3248, ATG-012, BI 1823911, D-1553, ERAS-3490, GDC-6036, GFH925, JAB-21822, JDQ-443, LY3537982, MRTX-1257, RMC-6291, and combinations thereof. In some embodiments, a first or second inhibitor of KRas can be selected from the group consisting of sotorasib, adragrasib, ARS-853, ARS-1620, ARS-3248, ATG-012, BI 1823911, D-1553, ERAS-3490, GDC-6036, GFH925, JAB-21822, JDQ-443, LY3537982, MRTX-1133, MRTX-1257, RMC-6291, RMC-6236, and combinations thereof.

In some embodiments, the methods provided herein include performing an assay on a sample (e.g., a tumor sample or a blood sample) obtained from the subject to determine whether the cancer in the subject has a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification). In some such embodiments, the method also includes administering to a subject determined to have a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification) a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof. In some embodiments, the method includes determining that a cancer in a subject has a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification) via an assay performed on a sample obtained from the subject. In such embodiments, the method also includes administering to a subject a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof. Some embodiments of these methods further include administering to the subject another anticancer agent (e.g., a second compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or immunotherapy).

In some embodiments of any of the methods or uses described herein, an assay is used to determine whether the cancer in the subject has a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification), using a sample (e.g., a tumor sample or a blood sample) from a subject can include, for example, next generation sequencing, immunohistochemistry, fluorescence microscopy, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof. Assays can utilize other detection methods known in the art for detecting a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification) (see, e.g., the references cited herein). In some embodiments, the sample is tumor biopsy sample (e.g., a paraffin-embedded biopsy sample) from the subject. In some embodiments, the subject is a subject suspected of having a KRas-associated cancer (e.g., mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))), a subject having one or more symptoms of a KRas-associated cancer (e.g., mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))), and/or a subject that has an increased risk of developing a KRas-associated cancer (e.g., mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))).

In some embodiments, a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification) can be identified using a liquid biopsy (variously referred to as a fluid biopsy or fluid phase biopsy). See, e.g., Karachialiou et al., “Real-time liquid biopsies become a reality in cancer treatment”, Ann. Transl. Med., 3(3):36, 2016. Liquid biopsy methods can be used to detect total tumor burden and/or the KRas dysregulation (e.g., the KRas mutation or amplification). Liquid biopsies can be performed on biological samples obtained relatively easily from a subject (e.g., via a simple blood draw) and are generally less invasive than traditional methods used to detect tumor burden and/or KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification). In some embodiments, liquid biopsies can be used to detect the presence of a KRas dysregulation (e.g., a KRas mutation or amplification) at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify a KRas dysregulation (e.g., KRas mutation (e.g., a KRas G12D mutation, a KRas G12R mutation, or a KRas G12V mutation (e.g., a KRas G12D mutation or a KRas G12V mutation))).

Also provided is a method for modulating (e.g., decreasing) KRas protein activity (e.g., dysregulated KRas protein activity (e.g., mutant KRas protein activity (e.g., KRas G12R mutant protein activity or G12V mutant protein activity))) in a cell, comprising contacting the cell with a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof. In some embodiments, the contacting is in vitro. In some embodiments, the contacting is in vivo. In some embodiments, the contacting is ex vivo. In some embodiments, the contacting is in vivo, wherein the method comprises administering an effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, to a subject having a cell having a KRas protein (e.g., a dysregulated KRas protein (e.g., a mutant KRas protein (e.g., a KRas G12D mutant protein, a KRas G12R mutant protein, or a KRas G12V mutant protein (e.g., a KRas G12D mutant protein or a KRas G12V mutant protein)))). In some embodiments, the contacting is ex vivo, wherein the method comprises contacting a cell from a subject having a KRas protein (e.g., a dysregulated KRas protein (e.g., a mutant KRas protein (e.g., a KRas G12D mutant protein, a KRas G12R mutant protein, or a KRas G12V mutant protein (e.g., a KRas G12D mutant protein or a KRas G12V mutant protein)))) with a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof. In some embodiments, the cell is a cancer cell. In some embodiments, the cell is a mammalian cell. In some embodiments, the cell is a mammalian cancer cell. In some embodiments, the cancer cell is any cancer as described herein. In some embodiments, the cancer cell is a KRas-associated cancer cell (e.g., a mutant KRas-associated cancer cell (e.g., a KRas G12D-associated cancer cell, a KRas G12R-associated cancer cell, or a KRas G12V-associated cancer cell (e.g., a KRas G12D-associated cancer cell or a KRas G12V-associated cancer cell))). As used herein, the term “contacting” refers to the bringing together of indicated moieties in an in vitro system, an in vivo system, or an ex vivo system. For example, “contacting” a KRas protein with a compound provided herein includes the administration of a compound provided herein to an individual or subject, such as a human, having a KRas protein, as well as, for example, introducing a compound provided herein into a sample containing a cellular or purified preparation containing the KRas protein.

Also provided herein is a method of inhibiting cell proliferation, in vitro, in vivo, or ex vivo, the method comprising contacting a cell with an effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition thereof as defined herein. In some embodiments, the cell has a KRas dysregulation. In some embodiments, the cell has a KRas mutation. In some embodiments, the cell has a KRas G12D mutation or a KRas G12V mutation. In some embodiments, the cell has a KRas G12D mutation. In some embodiments, the cell has a KRas G12R mutation. In some embodiments, the cell has a KRas G12V mutation. In some embodiments, the cell has a KRas amplification.

Further provided herein is a method of increasing cell death, in vitro, in vivo, or ex vivo, the method comprising contacting a cell with an effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or a pharmaceutical composition thereof as defined herein. Also provided herein is a method of increasing tumor cell death in a subject. The method comprises administering to the subject a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, in an amount effective to increase tumor cell death. In some embodiments, the cell has a KRas dysregulation. In some embodiments, the cell has a KRas mutation. In some embodiments, the cell has a KRas G12D mutation or a KRas G12V mutation. In some embodiments, the cell has a KRas G12D mutation. In some embodiments, the cell has a KRas G12R mutation. In some embodiments, the cell has a KRas G12V mutation. In some embodiments, the cell has a KRas amplification.

When employed as pharmaceuticals, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, can be administered in the form of pharmaceutical compositions as described herein.

Also provided herein is a method for inhibiting a KRas protein in a mammalian cell, the method comprising contacting the mammalian cell with an effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof. Also provided herein is a method for inhibiting a dysregulated KRas protein (e.g., a mutant KRas protein (e.g., a KRas G12D mutant protein, a KRas G12R mutant protein, or a KRas G12V mutant protein (e.g., a KRas G12D mutant protein or a KRas G12V mutant protein))) in a mammalian cell, the method comprising contacting the mammalian cell with an effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof. In some embodiments, the mammalian cell is ex vivo.

Also provided herein is a method of treating a subject having a cancer, wherein the method comprises:
administering a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, as a monotherapy or in conjunction with a first anticancer agent to the subject who has been administered one or more doses of the first anticancer agent to the subject for a period of time.

Also provided herein is a method of treating a subject having a cancer, wherein the method comprises:
(a) administering one or more doses of a first anticancer agent to the subject for a period of time;
(b) after (a), determining whether a cancer cell in a sample (e.g., a tumor sample or a blood sample) obtained from the subject has a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification); and
(c) administering a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, as a monotherapy or in conjunction with a second anticancer agent to the subject if the subject has been determined to have a cancer cell that has a KRas dysregulation (e.g., a KRas mutation or amplification); or
(d) administering additional doses of the first anticancer agent of step (a) to the subject if the subject has not been determined to have a cancer cell that has a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification).

Further provided herein is a method of treating a subject having a cancer, wherein the method comprises:
(a) determining whether a cancer cell in a sample (e.g., a tumor sample or a blood sample) obtained from a subject having a cancer and previously administered one or more doses of a first anticancer agent has a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification); and
(b) administering a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, as a monotherapy or in conjunction with a second anticancer agent to the subject if the subject has been determined to have a cancer cell that has a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification); or
(c) administering additional doses of the first anticancer agent to the subject if the subject has not been determined to have a cancer cell that has a KRas dysregulation (e.g., KRas mutation (e.g., a KRas G12D mutation, a KRas G12R mutation, or a KRas G12V mutation (e.g., a KRas G12D mutation or a KRas G12V mutation)) or amplification).

Also provided herein is a method of treating a subject having a cancer, wherein the method comprises:
(a) determining that a cancer cell in a sample (e.g., a tumor sample or a blood sample) obtained from a subject having a cancer and previously administered one or more doses of a first anticancer agent has a KRas dysregulation (e.g., a KRas mutation (e.g., a KRas G12D mutation, a KRas G12R mutation, or a KRas G12V mutation (e.g., a KRas G12D mutation or a KRas G12V mutation)) or amplification); and
(b) administering a therapeutically effective amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, as a monotherapy or in conjunction with a second anticancer agent to the subject.

In some embodiments of any of the methods described herein, the first anticancer agent can be a first KRas inhibitor.

When employed as pharmaceuticals, the compounds Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, can be administered in the form of pharmaceutical compositions as described herein.

### Combinations

In any of the indications described herein, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can be used as a monotherapy. In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, can be used prior to administration of an additional therapeutic agent or additional therapy. For example, a subject in need thereof can be administered one or more doses of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, for a period of time and then undergo at least partial resection of the tumor. In some embodiments, the treatment with one or more doses of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, reduces the size of the tumor (e.g., the tumor burden) prior to the at least partial resection of the tumor.

In some embodiments, a subject in need thereof can be administered one or more doses of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, for a period of time and under one or more rounds of radiation therapy. In some embodiments, the treatment with one or more doses of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, reduces the size of the tumor (e.g., the tumor burden) prior to the one or more rounds of radiation therapy.

In some embodiments, a subject has a cancer (e.g., a locally advanced or metastatic tumor) that is refractory or intolerant to standard therapy (e.g., administration of a chemotherapeutic agent, such as a first KRas inhibitor, a kinase inhibitor, immunotherapy, or radiation. In some embodiments, a subject has a cancer (e.g., a locally advanced or metastatic tumor) that is refractory or intolerant to prior therapy (e.g., administration of a chemotherapeutic agent, such as a first KRas inhibitor, a kinase inhibitor, immunotherapy, or radiation). In some embodiments, a subject has a cancer (e.g., a locally advanced or metastatic tumor) that has no standard therapy. In some embodiments, a subject is KRas inhibitor naïve. In some embodiments, a subject is not KRas inhibitor naïve. In some embodiments, a subject has undergone prior therapy. For example, treatment with surgery, radiation, a chemotherapeutic agent, an immunotherapy, a multi-kinase inhibitor (MKI), a KRas inhibitor, a RAF/MEK/PI3K pathway inhibitor, a MEK inhibitor, a Raf inhibitor, a YAP inhibitor, a proteasome inhibitor, a PI3K-AKT-mTOR pathway inhibitor, an ERK inhibitor, a pan-ErbB inhibitor, a MET inhibitor, a farnesyl transferase inhibitor, a FAK inhibitor, a HSP90 inhibitor, or a combination thereof.

In some embodiments of any the methods described herein, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is administered in combination with a therapeutically effective amount of at least one additional therapeutic agent selected from one or more additional therapies or therapeutic (e.g., chemotherapeutic) agents.

Non-limiting examples of additional therapeutic agents include: RAS pathway targeted therapeutic agents (e.g., Ras/RAF/MEK/PI3K pathway inhibitors or degraders, (e.g., Ras inhibitors or degraders, KRas-targeted therapeutic agents, SOS1 inhibitors or degraders, SOS1/Ras protein-protein interaction inhibitors, SHP2 inhibitors or degraders, PI3K-AKT-mTOR pathway inhibitors or degraders)), kinase-targeted therapeutics (e.g., MEK inhibitors or degraders, ERK inhibitors or degraders, Raf inhibitors or degraders (e.g., BRaf inhibitors or degraders), PI3K inhibitors or degraders, AKT inhibitors or degraders, mTOR inhibitors or degraders, CDK4/5 inhibitors or degraders, CDK4/6 inhibitors or degraders, MET inhibitors or degraders, FAK inhibitors or degraders, ErbB family inhibitors or degraders (e.g., EGFR inhibitors or degraders, Her2 inhibitors or degraders), Src inhibitors or degraders), mTORC1 inhibitors or degraders, YAP inhibitors or degraders, proteasome inhibitors or degraders, farnesyl transferase inhibitors or degraders, HSP90 inhibitors or degraders, PTEN inhibitors or degraders, signal transduction pathway inhibitors or degraders, checkpoint inhibitors, modulators of the apoptosis pathway (e.g., venetoclax, navitoclax, obataclax), chemotherapeutics, angiogenesis-targeted therapies, immune-targeted agents including immunomodulatory imide drugs (sometimes called “IMiDs” or “CELMoDs”), immunotherapy (e.g., anti-PD1, anti-PD-L1, anti-CTLA4, anti-LAG3, anti-TIM3, anti-B7-H3, anti-VISTA therapies, including antibodies (e.g., single-targeted antibodies targeting one or more of PD1, PD-L1, CTLA4, LAG3, TIM3, B7-H3, or VISTA; bispecific antibodies (including bispecific T cell engagers (BiTEs)) targeting one or more of PD1, PD-L1, CTLA4, LAG3, TIM3, B7-H3, or VISTA; and antibody-drug conjugates (ADCs) incorporating one or more of PD1, PD-L1, CTLA4, LAG3, TIM3, B7-H3, or VISTA) or antigen-binding fragments thereof, a PD-1 inhibitor, a PD-L1 inhibitor, or an ADOR2A inhibitor), cell-based therapeutics (e.g., adoptive cell therapy (e.g., CAR T therapy, cytokine-induced killer cells (CIKs), natural killer cells (e.g., CAR-modified NK cells)) or antibody-armed cell therapy), and radiotherapy. See also, e.g., the therapeutic agents listed in U.S. Publication No. 2021/0130303.

A “degrader” as used herein is a heterobifunctional molecule that induces degradation of a target protein, the degrader including a moiety that binds to the target protein and a moiety that binds to a ubiquitin E3 ligase (sometimes referred to as an E3 ligase or simply an E3), these two moieties being optionally separated by a linker. Such degraders are sometimes known as “PROTACs”.

A “Ras pathway targeted therapeutic agent” as used herein includes any compound exhibiting inactivation activity of any protein in a Ras pathway (e.g., kinase inhibition, allosteric inhibition, inhibition of dimerization, and/or induction of degradation). Non-limiting examples of a protein in a Ras pathway include any one of the proteins in the Ras-RAF-MAPK pathway or PI3K/AKT pathway such as Ras (e.g., KRas, HRas, and NRas), RAF, BRAF, MEK, ERK, PI3K, AKT, and mTOR. In some embodiments, a Ras pathway modulator can be selective for a protein in a Ras pathway, e.g., the Ras pathway modulator can be selective for Ras (also referred to as a Ras modulator). In some embodiments, a Ras modulator is a covalent inhibitor. In some embodiments, a Ras pathway targeted therapeutic agent is a “KRas pathway modulator.” A KRas pathway modulator includes any compound exhibiting inactivation activity of any protein in a KRas pathway (e.g., kinase inhibition, allosteric inhibition, inhibition of dimerization, and/or induction of degradation). Non-limiting examples of a protein in a KRas pathway include any one of the proteins in the KRas-RAF-MAPK pathway or PI3K/AKT pathway such as KRas, RAF, BRAF, MEK, ERK, PI3K, AKT, and mTOR. In some embodiments, a KRas pathway modulator is a KRas-targeted therapeutic agent. In some embodiments, the Ras pathway targeted therapeutic agent is a SOS1 inhibitor or a SHP2 inhibitor. Non-limiting examples of SOS1 inhibitors include MRTX-0902, and RMC-5845. Non-limiting examples of SHP2 inhibitors include batoprotafib (TNO-155), vociprotafib (RMC-4630), ARRY-558, BBP-398, ENT-03, ERAS-601, ET-0038, GDC-1971 (RLY-1971), GH-21, HS-10381, ICP-189, JAB-3068, JAB-3312, and SH-3809.

Non-limiting examples of KRas-targeted therapeutic agents (e.g., a first KRas inhibitor or a second KRas inhibitor) include a KRas-selective inhibitor, a Ras inhibitor, and an anti-KRas antibody. In some embodiments, the KRas inhibitor is a covalent inhibitor. In some embodiments, the KRas-targeted therapeutic agent is adagrasib, divarasib (GDC-6036), sotorasib, ARS-1620, ARS-3248, ARS-853, ASP-3082, ATG-012, BI-1701963, BI-1823911, BPI-421286, D-1553, ERAS-3490, GFH-925, JAB-21822, JDQ-443, LY-3537982, MRTX-1133, MRTX-1257, RMC-6236, RMC-6291, RSC-1255, or a combination thereof.

In some embodiments, the KRas-targeted therapeutic agent is an agent that inhibits the interaction between KRas and SOS1 or SHP2. Non-limiting examples of an agent that inhibits the interaction between SOS1 and KRas include BI-3406, BI-1701963, and BAY 293.

Additional KRas-targeted therapeutic agents (e.g., a first KRas inhibitor or a second KRas inhibitor) include those disclosed in International Publication Nos. WO 2021/104431; WO WO2021/119343; WO2021/113595; WO 2021/107160; WO 2016/161361; WO 2016/17262; WO 2020/035031; WO 2021/041671; WO 2016/077793; WO 2020/180768; WO 2021/092115; WO 2020/180770; U.S. Pat. Nos. 10,898,487; 10,829,487; 10,858,359; 10,561,655; 10,532,042; U.S. Publication Nos. 2021/0101870; 2019/0231805; 2020/0017517; 2020/0017511; 2020/0147058; 2021/0009577; and Hillig et al. PNAS. 2019; 116(7): 2551-2560.

Further non-limiting examples of Ras pathway-targeted therapeutic agents include BRAF inhibitors, MEK inhibitors, ERK inhibitors, PI3K inhibitors, AKT inhibitors, and mTOR inhibitors.

In some embodiments, the BRAF inhibitor is avutometinib, dabrafenib (e.g., dabrafenib mesylate, TAFINLAR®), encorafenib (BRAFTOVI™), naporafenib, sorafenib (e.g., sorafenib tosylate), vemurafenib (ZELBORAF®), ARQ 736, AZ304, BMS-908662 (XL281), C17071479-F, CHIR-265, FORE-8394, GDC-0879, GSK2118436, HLX-208, HM95573, LGX818, LXH254, PLX-3603, PLX-4720, PLX-8394, RAF265, R05126766, R05185426, or a combination thereof. In some embodiments, the BRAF inhibitor is avutometinib, dabrafenib (e.g., dabrafenib mesylate), encorafenib, naporafenib, sorafenib (e.g., sorafenib tosylate), vemurafenib, C17071479-F, CHIR-265, FORE-8394, HLX-208, or a combination thereof.

In some embodiments, the MEK inhibitor is avutometinib, binimetinib (MEKTOVI®, MEK162), cobimetinib (e.g., cobimetinib fumarate, COTELLIC®), mirdametinib, pimasertib, refametinib, selumetinib (e.g., selumetinib sulfate, AZD6244), trametinib (e.g., trametinib dimethyl sulfoxide, GSK-1120212 MEKINIST®), zapnometinib, hypothemycin, CI1040 (PD184352), CS3006, FCN-159, MSC1936369B, NFX-179, PD0325901, PD98059, RO5126766, SHR7390, TAK-733, WX-554, or a combination thereof. In some embodiments, the MEK inhibitor is avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide, GSK-1120212), zapnometinib, FCN-159, NFX-179, TAK-733, or a combination thereof.

In some embodiments, the ERK inhibitor is 25-OH-D3-3-BE (B3CD, bromoacetoxycalcidiol), 5-7-Oxozeaenol, 5-iodotubercidin, AEZ-131 (AEZS-131), AEZS-136, ASN007, AZ-13767370, BL-EI-001, CC-90003, FR148083, FR-180204, FRI-20 (ON-01060), GDC0994, GDC-0994 (RG-7482), KO-947, KO-947, LTT-462, LY-3214996, MK-8353 (SCH900353), ONC201SCH772984, ulixertinib (BVD-523), VTX-Ile, or a combination thereof. In some embodiments, the ERK inhibitor is rineterkib, ulixertinib, or a combination thereof.

In some embodiments, PI3K inhibitor is alpelisib (BYL719), apitolisib (GDC-0980), buparlisib (BKM120), copanlisib (ALIQOPA™, BAY80-6946), dactolisib (NVP-BEZ235, BEZ-235), gedatolisib (PF-05212384, PKI-587), omipalisib (GSK2126458, GSK458), pictilisib (GDC-0941), pilaralisib (XL147, SAR245408), rigosertib, serabelisib (TAK-117, MLN1117, INK 1117), sonolisib (PX-866), taselisib (GDC-0032, RG7604), voxtalisib (XL756, SAR245409), wortmannin, AMG 511, AMG319, ASN003, AZD8835, BGT-226 (NVP-BGT226), CH₅₁₃₂₇₉₉, CUDC-907, GDC-0077, GDC-0084 (RG7666), GS-9820, GSK1059615, GSK2636771, KIN-193 (AZD-6428), LY2023414, LY294002, PF-04691502, PI-103, PKI-402, PQR309, SAR260301, SF1126, VS-5584 (SB2343), WX-037, XL-765, ZSTK474, or a combination thereof. In some embodiments, the PI3K inhibitor is alpelisib, amdizalisib, apitolisib, bimiralisib, buparlisib, copanlisib (e.g., copanlisib dihydrochloride or a hydrate of copanlisib dihydrochloride), dactolisib, dezapelisib, dordaviprone, duvelisib (e.g., a hydrate of duvelisib), eganelisib, fimepinostat, gedatolisib, idelalisib, inavolisib, leniolisib (e.g., leniolisib phosphate), linperlisib, parsaclisib, paxalisib, risovalisib, seletalisib, serabelisib, sonolisib, tenalisib, umbralisib (e.g., umbralisib tosylate), zandelisib, PF-04691502, SHC-014748-M, TQ-B-3525, or a combination thereof.

In some embodiments, the AKT inhibitor is 2-[4-(2-aminoprop-2-yl)phenyl]-3-phenylquinoxaline, 3-oxo-tirucallic acid, A-443654, A-674563, afuresertib, API-1, ARQ092, AT13148, AT7867, AZD5363, BAY 1125976, boc-Phe-vinyl ketone, CCT128930, DC120, DM-PIT-1, edelfosine, erucylphophocholine, erufosine, GSK2141795, GSK690693, H-89, ipatasertib (GDC-0068, RG7440), lactoquinomycin, miltefosine (IMPADIVO®), MK-2206, N-(4-(5-(3-acetamidophenyl)-2-(2-aminopyridin-3-yl)-3H-imidazo[4,5-b]pyridin-3-yl)benzyl)-3-fluorobenzamide, NL-71-101, ONC201, OSU-A9, Perifosine (D-21266), PH-316, PHT-427, PIT-1, SR13668, TCN, TCN-P, triciribine (Triciribine Phosphate Monohydrate), uprosertib, wortmannin, or a combination thereof. In some embodiments, the AKT inhibitor is capivasertib (AZD-5363), miransertib (e.g., miransertib mesylate), pifusertib, uprosertib, BXT-10, or a combination thereof.

In some embodiments, the mTOR inhibitor is MLN0128, AZD-2014, CC-223, AZD2014, CC-115, everolimus (RAD001), temsirolimus (CCI-779), ridaforolimus (AP-23573), sirolimus (rapamycin), or a combination thereof. In some embodiments, the mTOR inhibitor is apitolisib, bimiralisib, dactolisib, everolimus, fosciclopirox (e.g., fosciclopirox sodium), gedatolisib, onatasertib, paxalisib, sapanisertib, sirolimus, sodium 2-hydroxylinoleate, temsirolimus, umirolimus, zandelisib, zotarolimus, BI-860585, CC-115, PF-04691502, or a combination thereof.

In some embodiments, the farnesyl transferase inhibitor is lonafarnib, tipifarnib, BMS-214662, L778123, L744832, and FTI-277. In some embodiments, the farnesyl transferase inhibitor is lonafarnib, tipifarnib, BMS-214662, or a combination thereof.

In some embodiments, a chemotherapeutic agent includes a DNA replication inhibitor (e.g., a DNA intercalator (e.g., an anthracycline)), a DNA crosslinker (e.g., cyclophosphamide, a mitomycin (e.g., mitomycin C), a platinum complex), a ribonucleotide-diphosphate reductase inhibitor (e.g., gemcitabine), or a topoisomerase inhibitor), an anti-microtubule agent (e.g., a taxane a vinca alkaloid, or eribulin), or a combination thereof.

Non-limiting examples of a taxane include paclitaxel, docetaxel, abraxane, and taxotere.

In some embodiments, the anthracycline is selected from daunorubicin, doxorubicin, epirubicin, idarubicin, and combinations thereof.

In some embodiments, the platinum-based agent is selected from carboplatin, cisplatin, oxaliplatin, nedplatin, triplatin tetranitrate, phenanthriplatin, picoplatin, satraplatin and combinations thereof.

In some embodiments, the chemotherapy is a platinum complex, a microtubule inhibitor (e.g., a microtubule destabilizer or a microtubule stabilizer), a topoisomerase inhibitor, or an antibody-drug conjugate including any thereof. In some embodiments, the platinum complex is carboplatin, cisplatin, lobaplatin, miriplatin, oxaliplatin, or a combination thereof. In some embodiments, the microtubule inhibitor is cabazitaxel, colchicine, desoxyepothilone B, docetaxel, eribulin, ixabepilone, nab-paclitaxel, paclitaxel, plinabulin, sabizabulin, tirbanibulin, vinblastine, vinflunine, vinorelbine, or a combination thereof. In some embodiments, the microtubule inhibitor is cabazitaxel, docetaxel, nab-paclitaxel, paclitaxel, or a combination thereof. In some embodiments, the topoisomerase inhibitor is aclarubicin, amsacrine, belotecan, camptothecin, daunorubicin, dexrazoxane, elliptinium, epirubicin, etoposide, gepotidacin, idarubicin, mitoxantrone, nemonoxacin, pirarubicin, pixantrone, razoxane, rubitecan, sobuzoxane, temozolomide, teniposide, topotecan, SN-38, or a combination thereof. In some embodiments, the hypomethylating agent is azacitidine, decitabine, or a combination thereof. In some embodiments, the chemotherapy is a platinum complex and a topoisomerase inhibitor (e.g., cisplatin and etoposide). In some embodiments, the antibody-drug conjugate including the microtubule inhibitor is belantamab mafodotin, brentuximab vedotin, cofetuzumab pelidotin, disitamab vedotin, enfortumab vedotin (e.g., enfortumab vedotin-ejfv, or a biosimilar thereof), mirvetuximab soravtansine (e.g., mirvetuximab soravtansine-gynx, or a biosimilar thereof), polatuzumab vedotin, telisotuzumab vedotin, tisotumab vedotin, trastuzumab emtansine (e.g., ado-trastuzumab emtansine, or a biosimilar thereof), tusamitamab ravtansine, upifitamab rilsodotin, zilovertamab vedotin, Alpha-Her2-pAF1-AS-269, BAT-8001, TAA-013, biosimilars thereof, or a combination thereof. In some embodiments, the antibody-drug conjugate including the microtubule inhibitor is enfortumab vedotin (e.g., enfortumab vedotin-ejfv, or a biosimilar thereof). In some embodiments, the antibody-drug conjugate including the microtubule inhibitor is mirvetuximab soravtansine (e.g., mirvetuximab soravtansine-gynx, or a biosimilar thereof). In some embodiments, the antibody-drug conjugate including the microtubule inhibitor is trastuzumab emtansine (e.g., ado-trastuzumab emtansine, or a biosimilar thereof). In some embodiments, the antibody-drug conjugate including the topoisomerase inhibitor is datopotamab deruxtecan, patritumab deruxtecan, sacituzumab govitecan (e.g., sacituzumab govitecan-hziy, or a biosimilar thereof), trastuzumab deruxtecan (fam-trastuzumab deruxtecan-nxki, or a biosimilar thereof), or a combination thereof. In some embodiments, the antibody-drug conjugate including the topoisomerase inhibitor is sacituzumab govitecan (e.g., sacituzumab govitecan-hziy, or a biosimilar thereof). In some embodiments, the antibody-drug conjugate including the topoisomerase inhibitor is trastuzumab deruxtecan (e.g., fam-trastuzumab deruxtecan-nxki, or a biosimilar thereof).

In some embodiments, the EGFR inhibitor is abivertinib, afatinib, alflutinib, almonertinib, amivantamab, befotertinib, bleomycetin, brigatinib, canertinib, cetuximab, dacomitinib, delphinidin, depatuxizumab, dovitinib, duligotumab, erlotinib, furmonertinib, futuximab, gefitinib, icotinib, imgatuzumab, lapatinib, lazertinib, lisocabtagene, mereletinib, mobocertinib, modotuximab, nazartinib, necitumumab, neratinib, nimotuzumab, olmutinib, osimertinib, panitumumab, pelitinib, pingyangmycin, poziotinib, pyrotinib, quercetin, sapitinib, tarloxotinib, tesevatinib, tomuzotuximab, vandetanib, varlitinib, zalutumumab, and zorifertinib. In some embodiments, the EGFR inhibitor is abivertinib, afatinib (e.g., afatinib dimaleate), alflutinib (e.g., alflutinib mesylate), almonertinib (e.g., almonertinib mesylate), befotertinib, brigatinib, canertinib, dacomitinib (e.g., dacomitinib monohydrate), dovitinib, erlotinib (e.g., erlotinib hydrochloride), gefitinib, icotinib, lapatinib (e.g., lapatinib ditosylate monohydrate), larotinib, lazertinib, limertinib, mobocertinib (e.g., mobocertinib succinate), nazartinib, neratinib (e.g., neratinib maleate), olmutinib, osimertinib (e.g., osimertinib mesylate), pelitinib, poziotinib, pyrotinib (e.g., pyrotinib maleate), ruserontinib (SKLB-1028), sapitinib, sunvozertinib, tesevatinib, vandetanib, varlitinib, zorifertinib, BIBW-2948, BPI-7711, HA-121-28, SH-1028, or a combination thereof.

In some embodiments, the PARP inhibitor is iniparib, niraparib, olaparib (LYNPARZA®), pamiparib (BGB-290), rucaparib, talazoparib, veliparib, 2X-121, ABT-767, BMN 673, BSI-201, CEP 9722, E7016, IMP4297, INO-1001, JPI-289, KU-0059436 (AZD2281), NOV1401, PF-01367338, and RBN-2397. In some embodiments, the PARP inhibitor is fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, NMS-03305293, or a combination thereof. In some embodiments, the PARP inhibitor is a PARP1 inhibitor. In some embodiments, the PARP1 inhibitor is saruparib (AZD5305), NMS-03305293, or a combination thereof.

Non-limiting examples of immunotherapy include immune checkpoint therapies. Non-limiting examples of immune checkpoint therapies include antibodies and/or inhibitors that target CTLA-4, PD-1, PD-L1, BTLA, LAG-3, ADORA2A, TIM-3, B7-H3, VISTA, IDO, and combinations thereof.

In some embodiments, the anti-CTLA4 therapy is abatacept (e.g., ORENCIA® (abatacept), or a biosimilar thereof), botensilimab, cadonilimab, erfonrilimab, gotistobart, ipilimumab (e.g., YERVOY® (ipilimumab), or a biosimilar thereof), nurulimab, quavonlimab, tremelimumab (ticilimumab) (e.g., IMIUDO® (tremelimumab), or a biosimilar thereof), volrustomig, vudalimab, zalifrelimab, BMS-986218, PSB-205, biosimilars thereof, or a combination thereof.

In some embodiments, the anti-PD1 therapy is balstilimab, budigalimab, cadonilimab, camrelizumab, cemiplimab (e.g., cemiplimab-rwlc, or a biosimilar thereof), cetrelimab, dostarlimab (e.g., dostarlimab-gxly, or a biosimilar thereof), ezabenlimab, geptanolimab, ivonescimab, nivolumab (e.g., OPDIVO® (nivolumab), or a biosimilar thereof), nofazinlimab, pembrolizumab (e.g., KEYTRUDA® (pembrolizumab), or a biosimilar thereof), penpulimab, pidilizumab, pimivalimab, prolgolimab, pucotenlimab, retifanlimab (e.g., retifanlimab-dlwr, or a biosimilar thereof), rilvegostomig, rosnilimab, rulonilimab, sasanlimab, serplulimab, sintilimab (e.g., TYVYT® (sintilimab), or a biosimilar thereof), spartalizumab, tebotelimab, tislelizumab, toripalimab, volrustomig, vudalimab, zimberelimab, QL-1604, HX-009, INCB-086550, RG-6139, BAT-1306, SG-001, biosimilars thereof, or a combination thereof.

In some embodiments, the anti-PD-L1 therapy is adebrelimab, atezolizumab (e.g., TECENTRIQ® (atezolizumab), or a biosimilar thereof), avelumab (e.g., BAVENCIO® (avelumab), or a biosimilar thereof), bintrafusp alfa, cosibelimab, danburstotug, durvalumab (e.g., TIFINZI® (durvalumab), or a biosimilar thereof), envafolimab (e.g., ENWEIDA® (envafolimab), or a biosimilar thereof), erfonrilimab, pacmilimab, socazolimab, sugemalimab (e.g., CEJEMLY® (sugemalimab), or a biosimilar thereof), A-167, APL-502, AUPM-170, BNT-311, SHR-1701, biosimilars thereof, or a combination thereof.

In some embodiments, the PD-L1 inhibitor is INCB-086550.

In some embodiments, the anti-LAG3 therapy is eftilagimod alfa, favezelimab, fianlimab, ieramilimab, INCAGN-02385, miptenalimab, relatlimab (e.g., relatlimab-rmbw, or a biosimilar thereof), tebotelimab, IBI-110, LBL-007, RG-6139, biosimilars thereof, or a combination thereof.

In some embodiments, the ADOR2A inhibitor is etrumadenant, inupadenant, istradefylline, mefloquine (e.g., mefloquine), taminadenant, CPI-444, PBF-999, or a combination thereof. In some embodiments, the ADOR2A inhibitor is etrumadenant, inupadenant, istradefylline, mefloquine (e.g., mefloquine), taminadenant, PBF-999, or a combination thereof.

In some embodiments, the anti-TIM3 therapy is cobolimab, sabatolimab (MBG-453), AZD-7789, INCAGN-02390, TQB-2618, or a combination thereof.

In some embodiments, the anti-B7-H3 therapy is omburtamab, enoblituzumab, or a combination thereof.

In some embodiments, the anti-VISTA therapy is onvatilimab (JNJ-61610588), HMBD-002, K01401-020, KVA-12.1, SNS-101, or a combination thereof.

In some embodiments, the IDO inhibitor (e.g., IDO1 and/or IDO2 inhibitor) is 3-deazaguanine, beta-lapachone, diindolylmethane, epacadostat, indole-3-carbinol, indoximod, sertaconazole (e.g., sertaconazole nitrate), or a combination thereof.

See, for example, Marin-Acevedo, et al., J Hematol Oncol. 11: 39 (2018).

In some embodiments, the additional therapy or therapeutic agent is a combination of atezolizumab and nab-paclitaxel.

Accordingly, also provided herein is a method of treating cancer, comprising administering to a subject in need thereof (a) a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, (b) an additional therapeutic agent, and (c) optionally at least one pharmaceutically acceptable carrier for simultaneous, separate or sequential use for the treatment of cancer, wherein the amounts of the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, and the additional therapeutic agent are together effective in treating the cancer.

In some embodiments, the additional therapeutic agent(s) includes any one of the above listed therapies or therapeutic agents which are standards of care in cancers wherein the cancer has a KRas dysregulation (e.g., a KRas mutation or amplification). In some embodiments, the additional therapeutic agent(s) includes any one of the above listed therapies or therapeutic agents which are standards of care in a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))).

These additional therapeutic agents may be administered with one or more doses of the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, or pharmaceutical composition thereof, as part of the same or separate dosage forms, via the same or different routes of administration, and/or on the same or different administration schedules according to standard pharmaceutical practice known to one skilled in the art.

Also provided herein is (i) a pharmaceutical composition for treating a cancer in a subject in need thereof, which comprises (a) a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, (b) at least one additional therapeutic agent (e.g., any of the exemplary additional therapeutic agents described herein or known in the art), and (c) optionally at least one pharmaceutically acceptable carrier for simultaneous, separate or sequential use for the treatment of cancer, wherein the amounts of the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, and of the additional therapeutic agent are together effective in treating the cancer; (ii) the use of such a composition for the preparation of a medicament for the treatment of cancer; and (iii) a commercial package or product comprising such a composition for simultaneous, separate or sequential use. In some embodiments, the cancer is a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))).

Accordingly, also provided herein is a method of treating a cancer, comprising administering to a subject in need thereof (a) a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, and (b) an additional therapeutic agent, wherein the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, and the additional therapeutic agent are administered simultaneously, separately or sequentially, wherein the amounts of the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, and the additional therapeutic agent are together effective in treating the cancer. In some embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, and the additional therapeutic agent are administered simultaneously as separate dosages. In some embodiments, compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, and the additional therapeutic agent are administered as separate dosages sequentially in any order, in jointly therapeutically effective amounts, e.g., in daily or intermittently dosages. In some embodiments, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, and the additional therapeutic agent are administered simultaneously as a combined dosage. In some embodiments, the cancer is a KRas-associated cancer (e.g., a mutant KRas-associated cancer (e.g., a KRas G12D-associated cancer, a KRas G12R-associated cancer, or a KRas G12V-associated cancer (e.g., a KRas G12D-associated cancer or a KRas G12V-associated cancer))).

The term “wild type” or “wild-type” describes a nucleic acid (e.g., a KRAS gene or a KRas mRNA) or protein (e.g., a KRas protein) sequence that is typically found in a subject that does not have a disease or disorder related to the reference nucleic acid or protein. Although a wild type nucleic acid or protein sequence is the sequence that is typically found in a subject that does not have a disease or disorder related to the reference nucleic acid or protein, it is not necessarily the case that a subject that has a disease or disorder related to the reference nucleic acid or protein lacks wild type sequence. For example, a subject with a gene duplication of the reference gene may have the wild type sequence but could still have a disease or disorder related to the reference nucleic acid or protein due to the duplication event. As another example, a subject with a disease or disorder related to the reference nucleic acid or protein may have one allele that encodes wild type protein, and another allele that encodes a mutant protein.

The term “wild type KRas” or “wild-type KRas” describes a KRas nucleic acid (e.g., a KRAS gene or a KRas mRNA) or protein (e.g., a KRas protein) that is found in a subject that does not have a KRas-associated disease, e.g., a KRas-associated cancer (and optionally also does not have an increased risk of developing a KRas-associated disease and/or is not suspected of having a KRas-associated disease), or is found in a cell or tissue from a subject that does not have a KRas-associated disease, e.g., a KRas-associated cancer (and optionally also does not have an increased risk of developing a KRas-associated disease and/or is not suspected of having a KRas-associated disease). Although a wild type KRas nucleic acid or protein sequence is the sequence that is typically found in a subject that does not have a KRas-associated disease or disorder, it is not necessarily the case that a subject that has a KRas-associated disease or disorder lacks the wild type KRas sequence. For example, a subject with a KRas gene duplication may have the wild type sequence but could still have a KRas-associated disease or disorder due to the duplication event. As another example, a subject with a KRas-associated disease or disorder may have one allele that encodes wild type KRas protein, and another allele that encodes a mutant KRas protein.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease or disorder to be treated and/or prevented.

In some embodiments, the subject is a pediatric subject.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

The term “activating mutation” in reference to KRas describes a mutation in a KRas gene that results in the expression of a KRas protein that has decreased GTPase activity and/or increased effector activation activity, e.g., as compared to a wild type KRas protein, e.g., when assayed under identical conditions. For example, an activating mutation can be a mutation in a KRAS gene that results in the expression of a KRas protein that has one or more (e.g., two, three, four, five, six, seven, eight, nine, or ten) amino acid substitutions (e.g., any combination of any of the amino acid substitutions described herein) that has decreased GTPase activity, e.g., as compared to a wild type KRas protein, e.g., when assayed under identical conditions. In another example, an activating mutation can be a mutation in a KRAS gene that results in the expression of a KRas protein that has one or more (e.g., two, three, four, five, six, seven, eight, nine, or ten) amino acids deleted, e.g., as compared to a wild type KRas protein, e.g., when assayed under identical conditions. In another example, an activating mutation can be a mutation in a KRAS gene that results in the expression of a KRas protein that has at least one (e.g., at least 2, at least 3, at least 4, at least 5, at least 6, at least 7, at least 8, at least 9, at least 10, at least 12, at least 14, at least 16, at least 18, or at least 20) amino acid inserted as compared to a wild type KRas protein, e.g., the exemplary wild type KRas protein described herein, e.g., when assayed under identical conditions. Additional examples of activating mutations are known in the art.

The term “preventing” as used herein means to delay the onset, recurrence or spread, in whole or in part, of the disease or condition as described herein, or a symptom thereof.

The term “regulatory agency” refers to a country's agency for the approval of the medical use of pharmaceutical agents with the country. For example, a non-limiting example of a regulatory agency is the U.S. Food and Drug Administration (FDA).

The phrase “therapeutically effective amount” means an amount of compound that, when administered to a subject in need of such treatment, is sufficient to (i) treat a KRas-associated disease or disorder (e.g., a mutant KRas-associated disease or disorder (e.g., a KRas G12D-associated cancer, a KRas G12R-associated cancer, or a KRas G12V-associated cancer (e.g., a KRas G12D-associated cancer or a KRas G12V-associated cancer)), (ii) attenuate, ameliorate, or eliminate one or more symptoms of the particular disease, disorder, or condition, or (iii) delay the onset of one or more symptoms of the particular disease, disorder, or condition described herein. The amount of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, that will correspond to such an amount will vary depending upon factors such as the particular compound, disease condition and its severity, the identity (e.g., weight) of the subject in need of treatment, but can nevertheless be routinely determined by one skilled in the art.

### Pharmaceutical Compositions and Administration

### General

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is administered as a pharmaceutical composition that includes the compound, or a pharmaceutically acceptable salt thereof, and one or more pharmaceutically acceptable excipients, and optionally one or more additional therapeutic agents as described herein.

In some embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, as described herein in the range of 0.005% to 100% with the balance made up from non-toxic excipient may be prepared. The contemplated compositions may contain 0.001%-100% of a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

### Routes of Administration and Composition Components

In some embodiments, the compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, or a pharmaceutical composition comprising a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salts thereof, can be administered to a subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

In some embodiments, a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, as described herein, or a pharmaceutical composition thereof, can be administered orally to a subject in need thereof. Without being bound by any particular theory, it is believed that oral dosing (e.g., versus IV dosing) can be preferred by patients for convenience, perception of efficacy, and/or past experience.

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, in the required amount in the appropriate solvent with various of the other ingredients enumerated above, as required, followed by filtered sterilization. Generally, dispersions are prepared by incorporating the various sterilized compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, are suitable for local delivery to the digestive or GI tract by way of oral administration (e.g., solid or liquid dosage forms).

Solid dosage forms for oral administration include capsules, tablets, pills, powders, and granules. In such solid dosage forms, the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives, or the like. In another solid dosage form, a powder, marume, solution or suspension (e.g., in propylene carbonate, vegetable oils, PEGs, poloxamer 124 or triglycerides) is encapsulated in a capsule (gelatin or cellulose base capsule). Unit dosage forms in which one or more compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or pharmaceutically acceptable salts thereof, provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients, such as tablets and capsules, sterility is not required. The USP/NF standard is usually sufficient.

In certain embodiments, solid oral dosage forms can further include one or more components that chemically and/or structurally predispose the composition for delivery of the compound of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)), or a pharmaceutically acceptable salt thereof, to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier, and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic, or amphoteric surfactant. As with other carriers or vehicles, an ointment base should be inert, stable, nonirritating, and non-sensitizing.

In any of the foregoing embodiments, pharmaceutical compositions described herein can include one or more one or more of the following: lipids, interbilayer crosslinked multilamellar vesicles, biodegradable poly(D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treated, and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 1 mg/kg; from about 0.1 mg/kg to about 0.5 mg/kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

The term “acceptable” with respect to a formulation, composition, or ingredient, as used herein, means having no persistent detrimental effect on the general health of the subject being treated.

“API” refers to an active pharmaceutical ingredient.

The term “excipient” or “pharmaceutically acceptable excipient” means a pharmaceutically acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid, and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The term “pharmacologically acceptable salts” is not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine, and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to a subject. Multiple techniques of administering a compound exist in the art including, but not limited to: rectal, oral, intravenous, aerosol, parenteral, ophthalmic, pulmonary, and topical administration.

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

Also provided herein are compounds useful as starting materials or intermediates in the preparation of compounds of Formula (A) (e.g., Formula (I) (e.g., Formula (I-a), (I-aa), (I-b), (I-bb), (I-c), (I-cc), (I-d), (I-dd), (I-e), (I-ee), (I-f), (I-g), or (I-h)), Formula (II) (e.g., (II-c), (II-cc), (II-d), (II-dd), (II-e), (II-ee), (II-f), (II-g), or (II-h)), or Formula (Aa)).

Provided herein are compounds of Formula (SI-Aa):

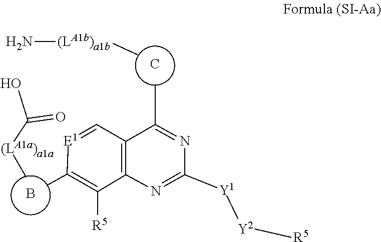

or salts thereof, wherein:
Ring C is a 4-10 membered heterocyclylene optionally substituted with 1-4 R⁷, wherein Ring C has one ring nitrogen atom, 0-1 ring oxygen atom, and no additional ring heteroatoms, wherein:
each R⁷ is independently selected from the group consisting of R^{a} and R^{b};
Ring B is selected from the group consisting of: C₆₋₁₀ arylene and 5-10 membered heteroarylene, each of which is optionally substituted with 1-3 substituents independently selected from the group consisting of: R^{a} and R^{b};
a1a and a1b are independently integers from 0 to 5, provided that a1a+a1b is 4, 5, or 6;
each L^{A1a} and L^{A1b} is an independently selected L^{A1};
each L^{A1} is independently selected from the group consisting of: —CH₂—, —CHR^{L}—, and —C(R^{L})₂—, wherein
each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, and C₁₋₆ alkyl optionally substituted with 1-6 R^{c};
R³ is:
a 5-8 membered heterocyclyl optionally substituted with 1-3 R^{a}, or
—NR^{d}R^{e} (e.g., —NMe₂); and
Y¹ is selected from the group consisting of: a bond, —N(H)—, —N(C₁₋₃ alkyl)-, —N(C₃₋₆ cycloalkyl)-, —O—, and —S(O)₀₋₂—;
Y² is a straight-chain C₁₋₆ alkylene optionally substituted with 1-6 R^{Y};
each R^{Y} is independently selected from the group consisting of: halo, cyano, —OH, oxo, C₁₋₆ alkoxy, C₁₋₆ haloalkoxy, C₁₋₆ alkyl, and C₁₋₆ haloalkyl, or
a pair of R^{Y} on the same or different carbon atom(s) taken together with the atom(s) connecting them form a C₃₋₆ cycloalkyl ring or 4-6 membered heterocyclyl ring, each of which is optionally substituted with 1-3 independently selected C₁₋₃ alkyl;
E¹ is N, CH, or CR⁶;
R⁵ is H or halo;
R⁶ are independently selected from the group consisting of:
(a) —H;
(b) halo;
(c) cyano;
(d) C₁₋₆ alkyl;
(e) C₁₋₆ haloalkyl;
(f) C₁₋₆ alkoxy;
(g) C₁₋₆ haloalkoxy;
(h) —(C₀₋₃ alkylene)-C₃₋₆ cycloalkyl, wherein the C₃₋₆ cycloalkyl portion is optionally substituted with 1-3 R^{g}; and
(i) —O—(C₀₋₃ alkylene)-C₃₋₆ cycloalkyl, wherein the C₃₋₆ cycloalkyl portion is optionally substituted with 1-3 R^{g};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
b is 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, cyano, —OH, —C₁₋₆ alkoxy, —C₁₋₆ haloalkoxy, —NH₂, —N(H)(C₁₋₃ alkyl), and —N(C₁₋₃ alkyl)₂.

In some embodiments of Formula (SI-Aa), Y¹ is —O—; Y² is —CH₂— or

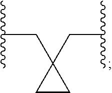 [SMILES: *CC1(C*)CC1]

R³ is a 5-8 membered heterocyclyl optionally substituted with 1-3 R^{a}; and R⁵ is halo. For example, Y² can be CH₂; R³ can be

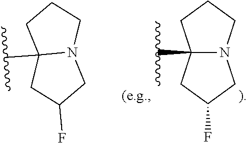 [SMILES: *C12CCCN1CC(F)C2 ; *[C@@]12CCCN1C[C@H](F)C2]

### and R⁵ can be —F.

In some embodiments of Formula (SI-Aa), E¹ is N, CH, or C-halo;
Ring C is selected from the group consisting of:

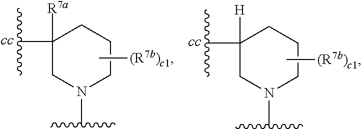

and

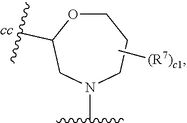

wherein:
c1 is 0, 1, or 2 (e.g., c1 is 0);
cc represents the point of attachment to -(L^{A1b})_{a1b}-;
each R^{7a} and R^{7b} is an independently selected R⁷ (e.g., R^{7a} is —OH); and
Ring B is selected from the group consisting of:

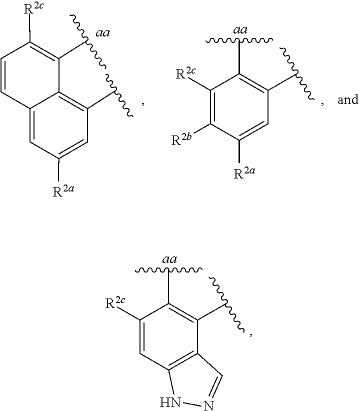

wherein R^{2a} is —OH or —NH₂; R^{2b} and R^{2c} are independently H or halo (e.g., —Cl); and aa represents the point of attachment to -(L^{A1a})ₐ₁ₐ-.

In some embodiments of Formula (SI-Aa), a1b is 2; and a1a is 2, 3, or 4. In some embodiments of Formula (SI-Aa), a1b is 1; and a1a is 3, 4, or 5.

In some embodiments of Formula (SI-Aa), each of L^{A1a} and L^{A1b} is CH₂.

Also provided herein is a method of preparing a compound of Formula (Aa) as depicted in Scheme 1:

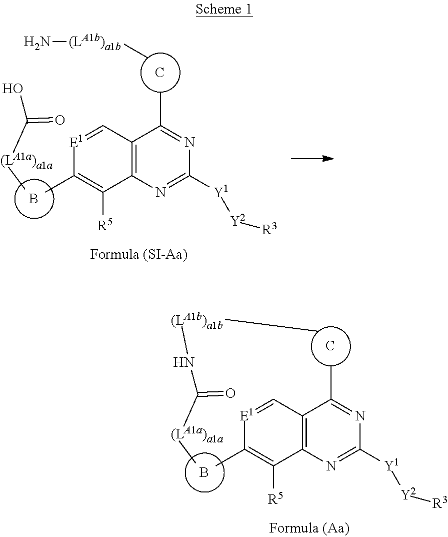

wherein the method comprises reacting a compound of Formula (SI-Aa), wherein Ring B, Ring C, L^{A1a}, a1a, L^{A1b}, a1b, E¹, R⁵, Y¹, Y², and R³ are as defined herein,
to form a compound of Formula (Aa), wherein Ring B, Ring C, L^{A1a}, a1a, L^{A1b} a1b, E¹, R⁵, Y¹, Y², and R³ are as defined herein.

In some embodiments, the method comprises reacting a compound of Formula (SI-Aa) under standard conditions for amide bond formation. Exemplary conditions for amide bond formation include those described in Chem. Soc. Rev., 2009, 38, 606-631. In some embodiments, the method comprises reacting a compound of Formula (SI-Aa) with a carboxylate activating agent (e.g., HATU) in presence of a base (e.g., NN-disopropylethylamine) and an appropriate solvent (e.g., DMF).

Provided herein are compounds of Formula (SII-Aa):

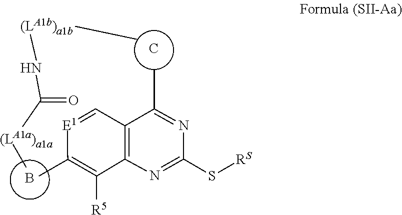

or salts thereof, wherein:
R^{S} is C₁₋₆ alkyl optionally substituted with 1-3 R^{c};
Ring C is a 4-10 membered heterocyclylene optionally substituted with 1-4 R⁷, wherein Ring C has one ring nitrogen atom, 0-1 ring oxygen atom, and no additional ring heteroatoms, wherein:
each R⁷ is independently selected from the group consisting of R^{a} and R^{b};
Ring B is selected from the group consisting of: C₆₋₁₀ arylene and 5-10 membered heteroarylene, each of which is optionally substituted with 1-3 substituents independently selected from the group consisting of: R^{a} and R^{b};
a1a and a1b are independently integers from 0 to 5, provided that a1a+a1b is 4, 5, or 6;
each L^{A1a} and L^{A1b} is an independently selected L^{A1};
each L^{A1} is independently selected from the group consisting of: —CH₂—, —CHR^{L}—, and —C(R^{L})₂—, wherein
each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, and C₁₋₆ alkyl optionally substituted with 1-6 R^{c};
E¹ is N, CH, or CR⁶;
R⁵ is H or halo;
R⁶ is selected from the group consisting of:
(a) —H;
(b) halo;
(c) cyano;
(d) C₁₋₆ alkyl;
(e) C₁₋₆ haloalkyl;
(f) C₁₋₆ alkoxy;
(g) C₁₋₆ haloalkoxy;
(h) —(C₀₋₃ alkylene)-C₃₋₆ cycloalkyl, wherein the C₃₋₆ cycloalkyl portion is optionally substituted with 1-3 R^{g}; and
(i) —O—(C₀₋₃ alkylene)-C₃₋₆ cycloalkyl, wherein the C₃₋₆ cycloalkyl portion is optionally substituted with 1-3 R^{g};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
b is 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, cyano, —OH, —C₁₋₆ alkoxy, —C₁₋₆ haloalkoxy, —NH₂, —N(H)(C₁₋₃ alkyl), and —N(C₁₋₃ alkyl)₂.

In some embodiments of Formula (SII-Aa), E¹ is N, CH, or C-halo;
Ring C is selected from the group consisting of:

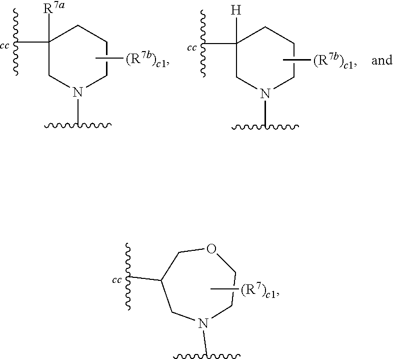

wherein:
c1 is 0, 1, or 2 (e.g., c1 is 0);
cc represents the point of attachment to -(L^{A1b})_{a1b}-;
each R^{7a} and R^{7b} is an independently selected R⁷ (e.g., R^{7a} is —OH); and
Ring B is selected from the group consisting of:

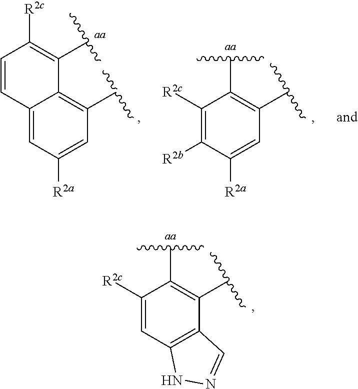

wherein R^{2a} is —OH or —NH₂; R^{2b} and R^{2c} are independently H or halo (e.g., —Cl); and aa represents the point of attachment to -(L^{A1a})ₐ₁ₐ-.

In some embodiments of Formula (SII-Aa), a1b is 2; and a1a is 2, 3, or 4. In some embodiments of Formula (SI-Aa), a1b is 1; and a1a is 3, 4, or 5.

In some embodiments of Formula (SII-Aa), each of L^{A1a} and L^{A1b} is CH₂.

Also provided herein is a method of preparing a compound of Formula (Aa) as depicted in Scheme 2:

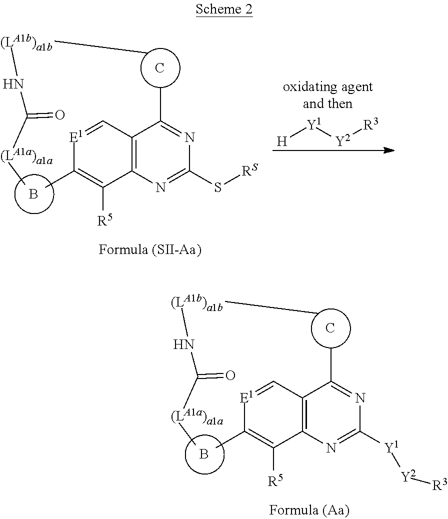

wherein the method comprises reacting a compound of Formula (SII-Aa), wherein Ring B, Ring C, L^{A1a}, a1a, L^{A1b}, a1b, E¹, R⁵, and R^{S} are as defined herein,
with an oxidating agent, followed by a compound of Formula R³—Y²—Y¹—H, wherein R³, Y², and Y¹ are as defined for Formula (Aa) (e.g., Y¹ is —O—),
to form a compound of Formula (Aa), wherein Ring B, Ring C, L^{A1a}, a1a, L^{A1b} a1b, E¹, R⁵, Y¹, Y², and R³ are as defined herein.

In some embodiments, the method comprises reacting the compound of Formula (SII-Aa) with an oxidating agent capable of oxidizing the sulfur atom bonded to R^{S}. For example, the oxidating agent can be mCPBA or Oxone. In some embodiments, the method comprises reacting the compound of Formula (SII-Aa) with an oxidating agent followed by a compound of Formula R³—Y²—OH in the presence of a base (e.g., NaH or NaHMDS).

Variations to the reaction sequences (e.g., introduction and/or removal of protecting groups, functional group transformations of intermediates and/or final products) in Schemes 1 and 2 are also contemplated. For example, in the preparation of Formula (Aa) compounds having an NH₂ group, this NH₂ group can be protected with one or two nitrogen protecting groups (e.g., the NH₂ can be protected as NBn₂ or NHBoc) during the reaction sequence, followed by the appropriate deprotection steps (e.g., HCl in 1,4-dioxane for NHBoc group; or Pd(OAc)₂/H₂ for NBn₂). As another example, in the preparation of Formula (Aa) compounds having a ring NH group, this ring NH group can be protected with a nitrogen protecting group (e.g., with a tetrahydropyran-2-yl) during the reaction sequence, followed by the appropriate deprotection steps (e.g., aqueous acidic conditions to remove the tetrahydropyran-2-yl group). As another example, in the preparation of Formula (Aa) compounds having an OH group, this OH group can be protected with an oxygen protecting group (e.g., methoxymethyl (MOM)) during the reaction sequence, followed by the appropriate deprotection steps.

Representative examples of nitrogen protecting groups include: acetyl (Ac), benzoyl (Bz), benzyl (Bn), benzyloxycarbonyl (Cbz), formyl, phenylsulfonyl, pivaloyl, tert-butoxycarbonyl (Boc), tert-butyl acetyl, ethyloxycarbonyl, trifluoroacetyl, triphenylmethyl (trityl), and triisopropylsilane. See: T. W. Greene and P. G. M. Wuts in “Protective groups in organic chemistry” John Wiley and Sons, 4th Edition, 2006.

Representative examples of oxygen protecting groups include: methoxymethyl (MOM), 2-methoxyethoxymethyl (MEM), benzyl (Bn), 4-methoxybenzyl (PMB), triethylsilyl TES), triisopropylsilyl (TIPS), trimethylsilyl (TMS), tert-butyldimethylsilyl (TBS), or tert-butyldiphenylsilyl TBDPS). See: T. W. Greene and P. G. M. Wuts in “Protective groups in organic chemistry” John Wiley and Sons, 4th Edition, 2006.

A person of ordinary skill in the art would understand that the final product and/or intermediates in Schemes 1 and/or 2 can be subjected to standard functional group transformation(s) to provide additional examples of compounds of Formula (Aa). For example, a compound of Formula (Aa) having an OH group can be converted into a second compound of Formula (Aa) wherein the corresponding OH group is replaced with an NH₂ group (e.g., by converting the OH group to OTf first).

### EXAMPLES

### General Analytical Methods:

Method A: Gemini NX-C18, 30×250 mm, Mobile phase A: 0.10% formic acid in H₂O (v/v), Mobile phase B: 0.1% formic acid in MeCN (v/v), Gradient: 10-100% Mobile phase B over 15 minutes.

LCMS Methods: Measurements were conducted on an Agilent single quadrupole mass spectrometer (MSD) coupled with an Agilent 1290 infinity UPLC system and ELSD (Agilent, Waldbronn, Germany). Sample were loaded and eluted from a Gemini NX-C18 column (100×2 mm, 3 μm pore size, 100 Å, Phenomenex, Torrance, CA, USA) at a flow rate of 0.7 mL/min. Column temperature was maintained at 40° C. Using 0.1% formic acid in water and 0.1% formic acid in acetonitrile as mobile phase A and B respectively, the compound was resolved using the gradient of 5% B for 0.3 min, 5-100% B in 2.6 min, and 100% B for 3.1 min. The ELSD parameters consisted of an evaporator temperature of 70° C., nebulizer temperature of 70° C., and gas flow of 1.6 SLM. The sing quadrupole mass spectrometer operated in the positive and negative ion mode with spray, fragmentor, and skimmer voltages of 3.5 kV, 135 V, and 35 V, respectively. The source gas temperature was maintained at 300° C. with a gas flow of 13 L/min.

### Example P. Preparation of Intermediates

### Synthesis of ethyl 3-(2-chloro-4-(methoxymethoxy)-6-(4,4,5,5-tetramethyl-1,3,2-dioxaborolan-2-yl)phenyl)propanoate

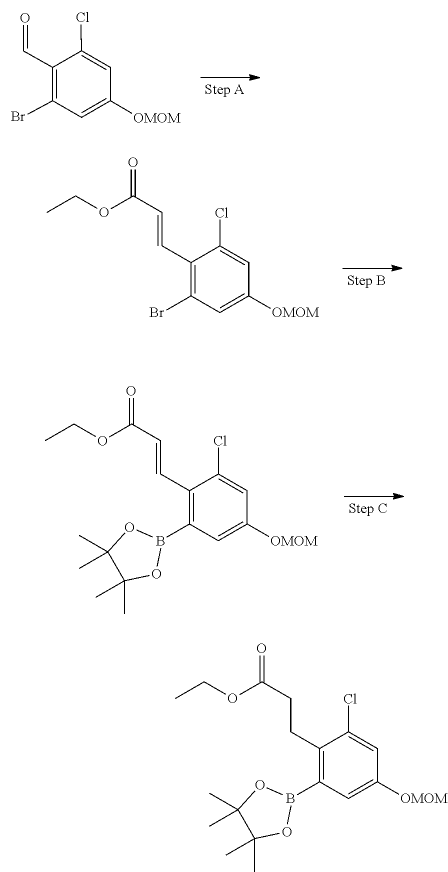

### Step A: Synthesis of ethyl (E)-3-(2-bromo-6-chloro-4-(methoxymethoxy)phenyl)acrylate

A solution of 2-bromo-6-chloro-4-(methoxymethoxy)benzaldehyde (2.00 g, 1.00 equiv., 7.16 mmol) and ethyl 2-(diethoxyphosphoryl)acetate (1.76 g, 1.10 equiv., 7.87 mmol) in THF (20 mL) was treated portion-wise with NaH (372 mg, 60 wt %, 1.30 equiv., 9.30 mmol). The mixture was stirred for 1 hour. The reaction mixture was diluted with EtOAc, washed with water and brine, dried (Na₂SO₄), filtered, and concentrated to afford ethyl (E)-3-(2-bromo-6-chloro-4-(methoxymethoxy)phenyl)acrylate (2.45 g, 98%).

¹H NMR (400 MHz, DMSO-d6) δ 7.60 (d, J=16.3 Hz, 1H), 7.42 (d, J=2.5 Hz, 1H), 7.29 (d, J=2.5 Hz, 1H), 6.48 (d, J=16.3 Hz, 1H), 5.29 (s, 2H), 4.22 (q, J=7.1 Hz, 2H), 3.38 (s, 3H), 1.27 (t, J=7.1 Hz, 3H)

### Step B: Synthesis of ethyl (E)-3-(2-chloro-4-(methoxymethoxy)-6-(4,4,5,5-tetramethyl-1,3,2-dioxaborolan-2-yl)phenyl)acrylate

A solution of ethyl (E)-3-(2-bromo-6-chloro-4-(methoxymethoxy)phenyl)acrylate (13.8 g, 1.00 equiv., 39.3 mmol), bis(pinacolato)diborane (20.0 g, 2.00 equiv., 78.7 mmol), potassium acetate (11.6 g, 3.00 equiv., 118 mmol), and Pd(dppf)Cl₂·CH₂Cl₂ (3.21 g, 0.100 equiv., 3.93 mmol) in 1,4-dioxane (100 mL) was warmed at 100° C. for 2 hours. The reaction mixture was diluted with ethyl acetate, washed with water and brine, dried (MgSO₄), filtered, and concentrated. The resulting residue was purified by flash column chromatography (ethyl acetate/heptane) to afford ethyl (E)-3-(2-chloro-4-(methoxymethoxy)-6-(4,4,5,5-tetramethyl-1,3,2-dioxaborolan-2-yl)phenyl)acrylate (15.0 g, 96%).

MS (ESI): m/z=397.2 [M+H]⁺.

### Step C: Synthesis of ethyl 3-(2-chloro-4-(methoxymethoxy)-6-(4,4,5,5-tetramethyl-1,3,2-dioxaborolan-2-yl)phenyl)propanoate

To a solution of ethyl (E)-3-(2-chloro-4-(methoxymethoxy)-6-(4,4,5,5-tetramethyl-1,3,2-dioxaborolan-2-yl)phenyl)acrylate (1.54 g, 1.00 equiv., 3.88 mmol) in ethanol (30 mL) was added Pd/C (155 mg, 0.375 equiv., 1.46 mmol). The reaction flask was evacuated (3×) and placed under hydrogen. The reaction mixture was stirred at ambient temperature for 4 hours. The reaction mixture was filtered through celite, and the filter cake was washed with ethanol. The filtrate was concentrated and purified by flash column chromatography (ethyl acetate/heptane) to afford ethyl 3-(2-chloro-4-(methoxymethoxy)-6-(4,4,5,5-tetramethyl-1,3,2-dioxaborolan-2-yl)phenyl)propanoate (250 mg, 627 μmol, 16.2%) as a colorless oil.

MS (ESI): m/z=399.2 [M+H]⁺.

### Synthesis of methyl 5-(2-chloro-4-(methoxymethoxy)-6-(4,4,5,5-tetramethyl-1,3,2-dioxaborolan-2-yl)phenyl)pentanoate

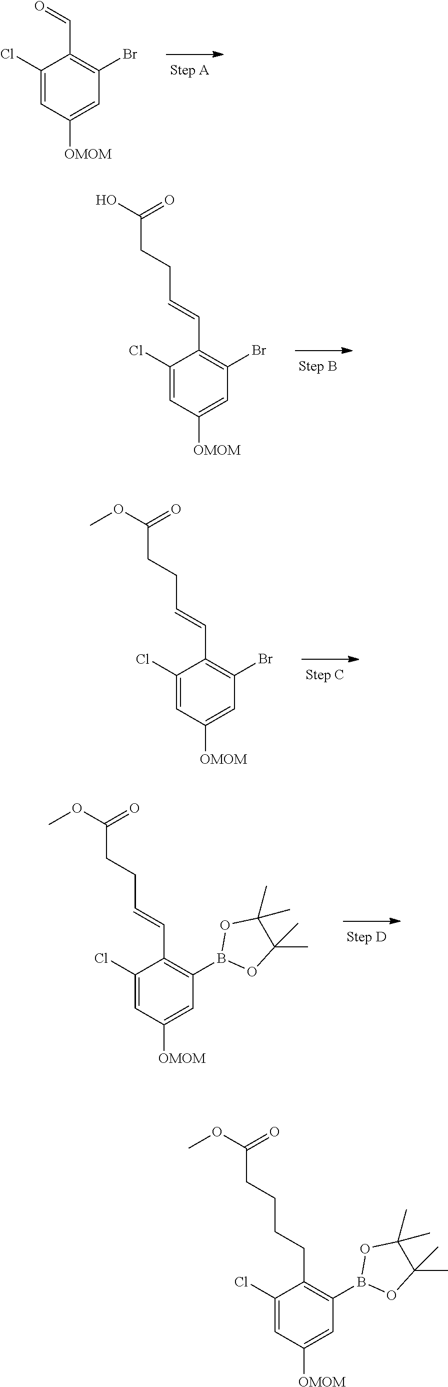

### Step A: Synthesis of (E)-5-(2-bromo-6-chloro-4-(methoxymethoxy)phenyl)pent-4-enoic acid

To a solution of (3-carboxypropyl)triphenylphosphonium bromide (5.8 g, 1.25 equiv., 13 mmol) and 2-bromo-6-chloro-4-(methoxymethoxy)benzaldehyde (3.0 g, 1.0 equiv., 11 mmol) in tetrahydrofuran (40 mL) at 0° C. was added potassium tert-butoxide (3.0 g, 27 mL, 1.0 M, 2.5 equiv., 27 mmol) dropwise over 10 minutes. The reaction mixture was then allowed to warm to room temperature. After 16 hours, the reaction mixture was diluted with water and extracted into ethyl acetate. The aqueous layer was adjusted to pH 3 by the addition of HCl (1 M) and extracted with ethyl acetate once more. The combined organic layers were further washed with brine, dried over sodium sulfate, filtered, and concentrated. Purification via flash column chromatography using a gradient of 0-100% B (A: heptane; B: EtOAc) gave (E)-5-(2-bromo-6-chloro-4-(methoxymethoxy)phenyl)pent-4-enoic acid (2.42 g, 64% yield).

MS (ESI): m/z=349.0 [M+H]⁺.

### Step B: Synthesis of methyl (E)-5-(2-bromo-6-chloro-4-(methoxymethoxy)phenyl)pent-4-enoate

To a solution of (E)-5-(2-bromo-6-chloro-4-(methoxymethoxy)phenyl)pent-4-enoic acid (3.21 g, 1 equiv., 9.18 mmol) and cesium carbonate (7.48 g, 2.5 equiv., 23.0 mmol) in acetone (30 mL) was added iodomethane (1.95 g, 861 μL, 1.5 equiv., 13.8 mmol). The reaction mixture was heated to 60° C. After 1 hour, the reaction mixture was filtered through celite and concentrated. Purification via flash column chromatography using a gradient of 0-80% B (A: heptane; B: EtOAc) gave methyl (E)-5-(2-bromo-6-chloro-4-(methoxymethoxy)phenyl)pent-4-enoate (3.22 g, 96% yield).

MS (ESI): m/z=363.1 [M+H]⁺.

### Step C: Synthesis of methyl (E)-5-(2-chloro-4-(methoxymethoxy)-6-(4,4,5,5-tetramethyl-1,3,2-dioxaborolan-2-yl)phenyl)pent-4-enoate

To a solution of methyl (E)-5-(2-bromo-6-chloro-4-(methoxymethoxy)phenyl)pent-4-enoate (3.11 g, 1 equiv., 8.55 mmol) in 1,4-dioxane (50 mL) was added potassium acetate (2.52 g, 3 equiv., 25.7 mmol), bis(pinacolato)diboron (4.34 g, 2 equiv., 17.1 mmol), and Pd(dppf)Cl₂·CH₂Cl₂ (698 mg, 0.1 equiv., 855 μmol). The reaction mixture was heated to 100° C. After 1 hour, the reaction mixture was filtered through celite and concentrated. Purification via flash column chromatography using a gradient of 0-25% B (A: heptane; B: EtOAc) gave methyl (E)-5-(2-chloro-4-(methoxymethoxy)-6-(4,4,5,5-tetramethyl-1,3,2-dioxaborolan-2-yl)phenyl)pent-4-enoate (2.84 g, 81% yield).

MS (ESI): m/z=411.5 [M+H]⁺.

### Step D: Synthesis of methyl 5-(2-chloro-4-(methoxymethoxy)-6-(4,4,5,5-tetramethyl-1,3,2-dioxaborolan-2-yl)phenyl)pentanoate

To a solution of methyl (E)-5-(2-chloro-4-(methoxymethoxy)-6-(4,4,5,5-tetramethyl-1,3,2-dioxaborolan-2-yl)phenyl)pent-4-enoate (1050 mg, 1 equiv., 2.557 mmol) in methanol (15 mL) was added Pd/C (105 mg, 0.386 equiv., 987 μmol). The flask was evacuated (3×) and placed under hydrogen (balloon). The reaction mixture was stirred at room temperature. After 90 minutes, the reaction mixture was filtered through celite, and the filter pad was rinsed with methanol. The filtrate was concentrated to give methyl 5-(2-chloro-4-(methoxymethoxy)-6-(4,4,5,5-tetramethyl-1,3,2-dioxaborolan-2-yl)phenyl)pentanoate (717 mg, 68%), which was immediately used in the following step.

MS (ESI): m/z=435.3 [M+Na]⁺.

### Synthesis of methyl 5-(4-(bis(4-methoxybenzyl)amino)-2-chloro-6-(4,4,5,5-tetramethyl-1,3,2-dioxaborolan-2-yl)phenyl)pentanoate

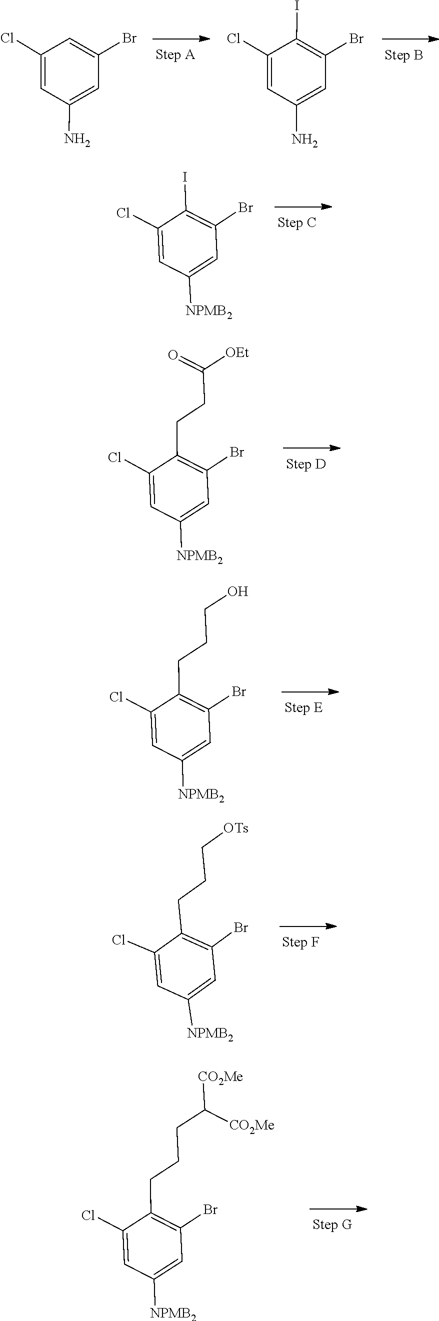

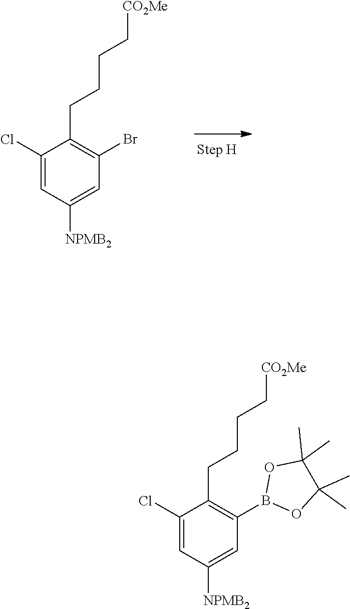

### Step A: Synthesis of 3-bromo-5-chloro-4-iodoaniline

To a solution of 3-bromo-5-chloro-aniline (40 g, 194 mmol, 1 equiv.) in DMF (450 mL) was added 1-iodopyrrolidine-2,5-dione (48.0 g, 213 mmol, 1.10 equiv.). The reaction mixture was stirred at room temperature for 2 hours. The reaction mixture was partitioned between H₂O (450 mL) and ethyl acetate (1350 mL). The organic phase was separated, washed with NaCl (300 mL×3), dried over Na₂SO₄, filtered, and concentrated under reduced pressure to give a residue. The residue was purified by prep-HPLC (basic) to afford 3-bromo-5-chloro-4-iodoaniline (40 g, 114 mmol, 59% yield) as a brown solid.

MS (ESI): m/z=331.9 [M+H]⁺.

### Step B: Synthesis of 3-bromo-5-chloro-4-iodo-N,N-bis(4-methoxybenzyl)aniline

To a solution of 3-bromo-5-chloro-4-iodoaniline (40 g, 120 mmol, 1 equiv.) in DMF (450 mL) was added NaH (14.4 g, 361 mmol, 60% purity, 3 equiv.) and PMBCl (47.1 g, 301 mmol, 2.5 equiv.). The reaction mixture was stirred at room temperature for 2 hours. The reaction mixture was quenched by the addition of NH₄Cl (450) mL at 0° C. and then was diluted with H₂O (450 mL) and extracted with ethyl acetate (450 mL×3). The combined organic layers were washed with NaCl (300 mL×3), dried over Na₂SO₄, filtered, and concentrated under reduced pressure to give a residue. The residue was purified by flash column chromatography (ethyl acetate/petroleum ether) to afford 3-bromo-5-chloro-4-iodo-N,N-bis(4-methoxybenzyl)aniline (52 g, 87.2 mmol, 72% yield) as a white solid.

MS (ESI): m/z=571.1 [M+H]⁺.

### Step C: Synthesis of ethyl 3-(4-(bis(4-methoxybenzyl)amino)-2-bromo-6-chlorophenyl)propanoate

A mixture of 3-bromo-5-chloro-4-iodo-N,N-bis(4-methoxybenzyl)aniline (36 g, 62.9 mmol, 1 equiv.), 3,3-diethoxyprop-1-ene (9.82 g, 75.4 mmol, 11.5 mL, 1.2 equiv.), tetrabutylammonium chloride (17.5 g, 62.9 mmol, 17.6 mL, 1 equiv.), DIPEA (16.3 g, 126 mmol, 21.9 mL, 2 equiv.), and Pd(OAc)₂ (706 mg, 3.14 mmol, 0.05 equiv.) in DMF (400 mL) was degassed and purged with N₂ three times. The mixture was stirred at 90° C. for 4 hours under N₂ atmosphere. The reaction mixture was partitioned between H₂O (1000 mL) and ethyl acetate (3000 mL). The organic phase was separated, washed with NaCl (500 mL×3), dried over Na₂SO₄, filtered, and concentrated under reduced pressure to give a residue. The residue was purified by flash column chromatography (ethyl acetate/petroleum ether) to afford ethyl 3-(4-(bis(4-methoxybenzyl)amino)-2-bromo-6-chlorophenyl)propanoate (32 g, 52.1 mmol, 82.8% yield) as a brown solid.

MS (ESI): m/z=546.2 [M+H]⁺.

### Step D: Synthesis of 3-(4-(bis(4-methoxybenzyl)amino)-2-bromo-6-chlorophenyl)propan-1-ol

A solution of ethyl 3-(4-(bis(4-methoxybenzyl)amino)-2-bromo-6-chlorophenyl)propanoate (22 g, 1 equiv., 40 mmol) in THF (250 mL) under nitrogen was treated with DIBAL-H (11 g, 80 mL, 1 M, 2 equiv., 80 mmol). The mixture was stirred at room temperature for 2 hours. The reaction mixture was slowly quenched by the addition of H₂O (30 mL), diluted with H₂O, and extracted with ethyl acetate (50 mL×3). The organic extracts were dried over Na₂SO₄, filtered, and concentrated under reduced pressure to give a residue. The residue was purified by flash column chromatography (ethyl acetate/petroleum ether) to afford 3-(4-(bis(4-methoxybenzyl)amino)-2-bromo-6-chlorophenyl)propan-1-ol (13 g, 18 mmol, 46%) as a yellow oil.

MS (ESI): m/z=504.0 [M+H]⁺.

### Step E: Synthesis of 3-(4-(bis(4-methoxybenzyl)amino)-2-bromo-6-chlorophenyl)propyl 4-methylbenzenesulfonate

A solution of 3-(4-(bis(4-methoxybenzyl)amino)-2-bromo-6-chlorophenyl)propan-1-ol (10.8 g, 1 equiv., 20.2 mmol) in pyridine (100 mL) under nitrogen at room temperature was treated with 4-methylbenzenesulfonyl chloride (4.62 g, 1.2 equiv., 24.3 mmol). The mixture was stirred for 2 hours. The reaction mixture was quenched by HCl (1 M, 30 mL), diluted with H₂O (50 mL), and extracted with ethyl acetate (50 mL×3). The organic extracts were dried over Na₂SO₄, filtered, and concentrated under reduced pressure to give a residue. The residue was purified by flash column chromatography (ethyl acetate/petroleum ether) to afford 3-(4-(bis(4-methoxybenzyl)amino)-2-bromo-6-chlorophenyl)propyl 4-methylbenzenesulfonate (7.4 g, 10 mmol, 51%) as a yellow oil.

MS (ESI): m/z=657.9 [M+H]⁺.

### Step F: Synthesis of dimethyl 2-(3-(4-(bis(4-methoxybenzyl)amino)-2-bromo-6-chlorophenyl)propyl)malonate

A solution of 3-(4-(bis(4-methoxybenzyl)amino)-2-bromo-6-chlorophenyl)propyl 4-methylbenzenesulfonate (7.40 g, 1 equiv., 11.2 mmol), dimethyl malonate (1.93 g, 1.3 equiv., 14.6 mmol), and sodium iodide (337 mg, 0.2 equiv., 2.25 mmol) in THF (30 mL) under nitrogen was treated with NaH (350 mg, 1.3 equiv., 14.6 mmol). The mixture was stirred at 65° C. for 5 hours. The reaction mixture was quenched by saturated aqueous NH₄Cl (50 mL), diluted with H₂O (50 mL), and extracted with ethyl acetate (50 mL×3). The organic extracts were dried over Na₂SO₄, filtered, and concentrated under reduced pressure to give a residue. The residue was purified by flash column chromatography (ethyl acetate/petroleum ether) to afford dimethyl 2-(3-(4-(bis(4-methoxybenzyl)amino)-2-bromo-6-chlorophenyl)propyl)malonate (6.8 g, 1.9 mmol, 45%) as a yellow oil.

MS (ESI): m/z=618.0 [M+H]⁺.

### Step G: Synthesis of methyl 5-(4-(bis(4-methoxybenzyl)amino)-2-bromo-6-chlorophenyl)pentanoate

A solution of dimethyl 2-(3-(4-(bis(4-methoxybenzyl)amino)-2-bromo-6-chlorophenyl)propyl)malonate (6.80 g, 1 equiv., 11.1 mmol), lithium chloride (921 mg, 2 equiv., 22 mmol), and water (2.0 mg, 0.01 equiv., 111 μmol) in DMSO (40 mL) was degassed and purged with N₂ three times. The mixture was stirred at 160° C. for 3 hours under N₂ atmosphere. The reaction mixture was diluted with H₂O (50 mL) and extracted with ethyl acetate (50 mL×3), dried over Na₂SO₄, filtered and concentrated under reduced pressure to give a residue. The residue was purified by flash column chromatography (ethyl acetate/petroleum ether) to afford methyl 5-(4-(bis(4-methoxybenzyl)amino)-2-bromo-6-chlorophenyl)pentanoate (3.53 g, 4.6 mmol, 81%) as a yellow oil.

MS (ESI): m/z=560.1 [M+H]⁺.

### Step H: Synthesis of methyl 5-(4-(bis(4-methoxybenzyl)amino)-2-chloro-6-(4,4,5,5-tetramethyl-1,3,2-dioxaborolan-2-yl)phenyl)pentanoate

A mixture of methyl 5-(4-(bis(4-methoxybenzyl)amino)-2-bromo-6-chlorophenyl)pentanoate (3.32 g, 1 equiv., 5.92 mmol), bis(pinacolato)diborane (1.80 g, 1.2 equiv., 7.10 mmol), Pd(dppf)Cl₂·CH₂Cl₂ (483 mg, 0.1 equiv., 592 μmol), and potassium acetate (1.74 g, 3 equiv., 17.8 mmol) in 1,4-dioxane (30 mL) was degassed and purged with N₂ three times. The mixture was stirred at 100° C. for 2 hours under N₂ atmosphere. The reaction mixture was diluted with ethyl acetate, filtered, and concentrated under reduced pressure to give a residue. The residue was purified by flash column chromatography (ethyl acetate/petroleum ether) to afford methyl 5-(4-(bis(4-methoxybenzyl)amino)-2-chloro-6-(4,4,5,5-tetramethyl-1,3,2-dioxaborolan-2-yl)phenyl)pentanoate (2.16 g, 3.0 mmol, 51%) as a yellow solid.

MS (ESI): m/z=608.2 [M+H]⁺.

### Synthesis of 4-(3-azidopropyl)-3-chloro-5-(4,4,5,5-tetramethyl-1,3,2-dioxaborolan-2-yl)phenol

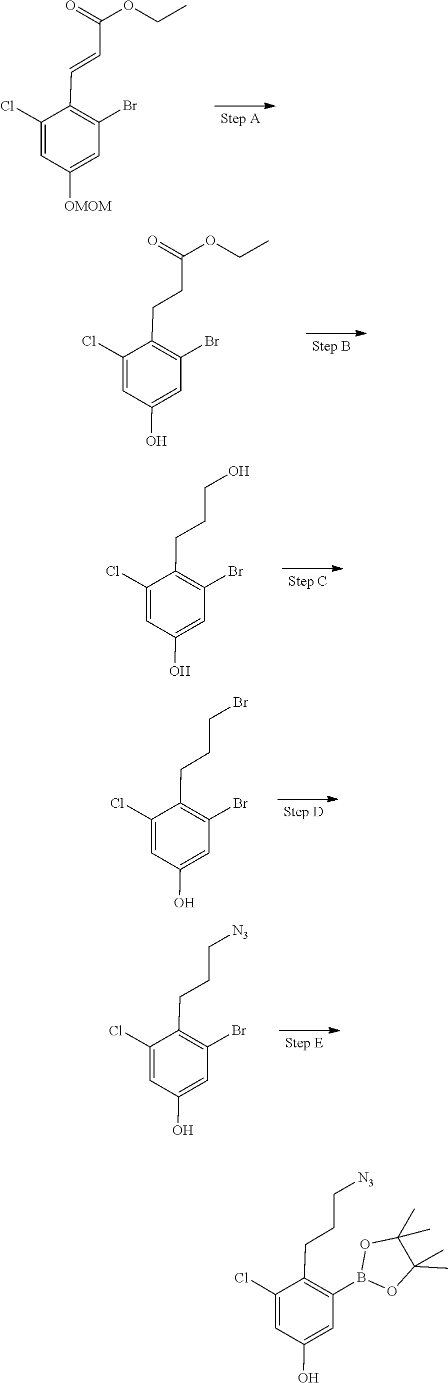

### Step A: Synthesis of ethyl 3-(2-bromo-6-chloro-4-hydroxyphenyl)propanoate

A solution of ethyl (E)-3-(2-bromo-6-chloro-4-(methoxymethoxy)phenyl)acrylate (5000 mg, 1 equiv., 14.3 mmol) and platinum(IV) oxide (650 mg, 0.2 equiv., 2.86 mmol) in EtOH (50 mL) was evacuated and back-filled with hydrogen two times. The mixture was stirred at room temperature under a hydrogen atmosphere overnight. The reaction mixture was filtered through a pad of celite, and the filtrate was concentrated to afford ethyl 3-(2-bromo-6-chloro-4-hydroxyphenyl)propanoate (4.39 g, 14.3 mmol, 100%), which was used directly in the next step without purification.

MS (ESI): m/z=305.1 [M−H]⁻.

### Step B: Synthesis of 3-bromo-5-chloro-4-(3-hydroxypropyl)phenol

A solution of ethyl 3-(2-bromo-6-chloro-4-hydroxyphenyl)propanoate (4399 mg, 1 equiv., 14.3 mmol) in THF (100 mL) at room temperature was treated dropwise with lithium borohydride in THF (467 mg, 10.7 mL, 2.0 M, 1.5 equiv., 21.5 mmol) followed by methanol (917 mg, 1.16 mL, 2 equiv., 28.6 mmol) over 5 minutes. The reaction mixture was stirred at room temperature for 18 hours. Additional lithium borohydride in THF (467 mg, 10.7 mL, 2.0 M, 1.5 equiv., 21.5 mmol) and methanol (917 mg, 1.16 mL, 2 equiv., 28.6 mmol) was added, and the reaction mixture was stirred at room temperature for 2 hours. Additional lithium borohydride in THF (467 mg, 10.7 mL, 2.0 M, 1.5 equiv., 21.5 mmol) and methanol (917 mg, 1.16 mL, 2 equiv., 28.6 mmol) were added, and the reaction mixture was stirred at room temperature for 3 days. The reaction mixture was diluted with ethyl acetate (250 mL), washed with water (50 mL) and saturated potassium phosphate monobasic (50 mL), dried (MgSO₄), filtered, and concentrated. The resulting residue was purified by flash column chromatography (ethyl acetate/heptane) to afford 3-bromo-5-chloro-4-(3-hydroxypropyl)phenol (1.52 g, 5.72 mmol, 40%).

MS (ESI): m/z=262.9 [M−H]⁻.

### Step C: Synthesis of 3-bromo-4-(3-bromopropyl)-5-chlorophenol

A solution of 3-bromo-5-chloro-4-(3-hydroxypropyl)phenol (1.12 g, 1 equiv., 4.21 mmol) in DCM (20 mL) at room temperature was treated with triphenylphosphine (1.22 g, 1.1 equiv., 4.63 mmol) and carbon tetrabromide (1.54 g, 1.1 equiv., 4.63 mmol). The mixture was stirred for 18 hours. Triphenylphosphine (1.22 g, 1.1 equiv., 4.63 mmol) and carbon tetrabromide (1.54 g, 1.1 equiv., 4.63 mmol) was added, and the reaction mixture was stirred for 24 hours. The reaction mixture was concentrated and purified by flash column chromatography (ethyl acetate/heptane) to afford 3-bromo-4-(3-bromopropyl)-5-chlorophenol (0.427 g, 1.30 mmol, 31%).

MS (ESI): m/z=325.0 [M−H]⁻.

### Step D: Synthesis of 4-(3-azidopropyl)-3-bromo-5-chlorophenol

A solution of 3-bromo-4-(3-bromopropyl)-5-chlorophenol (425 mg, 1 equiv., 1.29 mmol) in DMF (10 mL) was treated with sodium azide (421 mg, 5 equiv., 6.47 mmol). The mixture was stirred at 70° C. for 40 hours. The reaction mixture was concentrated, and the resulting residue was purified by flash column chromatography (ethyl acetate/heptane) to afford 4-(3-azidopropyl)-3-bromo-5-chlorophenol (335 mg, 1.15 mmol, 89.1%).

MS (ESI): m/z=287.9 [M−H]⁻.

### Step E: Synthesis of 4-(3-azidopropyl)-3-chloro-5-(4,4,5,5-tetramethyl-1,3,2-dioxaborolan-2-yl)phenol

A solution of 4-(3-azidopropyl)-3-bromo-5-chlorophenol (130 mg, 1 equiv., 447 μmol) in 1,4-dioxane (4 mL) was treated with bis(pinacolato)diborane (170 mg, 1.5 equiv., 671 μmol), PdCl₂(dppf)·CH₂Cl₂ (36.5 mg, 0.1 equiv., 44.7 μmol) and potassium acetate (132 mg, 3 equiv., 1.34 mmol), degassed with nitrogen, and warmed at 50° C. for 3 days. The reaction mixture was filtered and purified by HPLC (Method A) to afford 4-(3-azidopropyl)-3-chloro-5-(4,4,5,5-tetramethyl-1,3,2-dioxaborolan-2-yl)phenol (33 mg, 98 μmol, 22%) as a brown oil.

MS (ESI): m/z=338.1 [M+H]⁺.

### Synthesis of methyl 5-(4-amino-5-fluoro-2-(4,4,5,5-tetramethyl-1,3,2-dioxaborolan-2-yl)phenyl)pentanoate

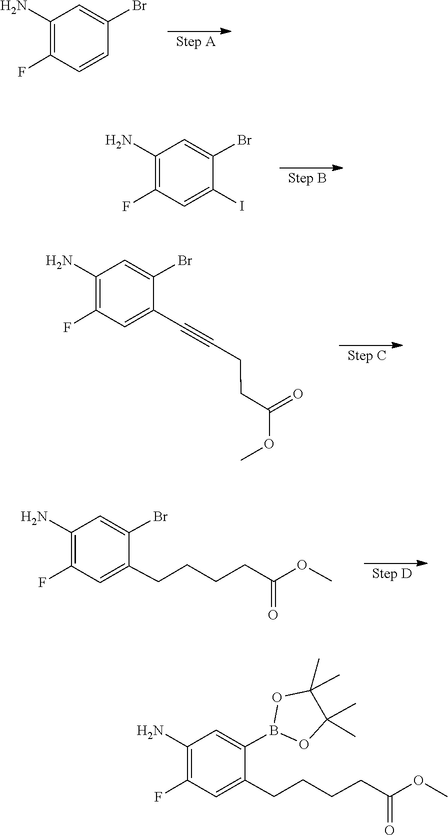 [SMILES: Nc1cc(Br)ccc1F ; Nc1cc(Br)c(I)cc1F ; COC(=O)CCC#Cc1cc(F)c(N)cc1Br ; COC(=O)CCCCc1cc(F)c(N)cc1Br ; COC(=O)CCCCc1cc(F)c(N)cc1B1OC(C)(C)C(C)(C)O1]

### Step A: Synthesis of 5-bromo-2-fluoro-4-iodoaniline

NIS (12.4 g, 1.05 equiv., 55.3 mmol) was added in portions to a solution of 5-bromo-2-fluoroaniline (10.0 g, 1 equiv., 52.6 mmol) in acetonitrile (200 mL) at room temperature over 1 hour under nitrogen gas. The resulting mixture was stirred at room temperature for 16 hours. The reaction mixture was concentrated, taken up in water (100 mL), and extracted with ethyl acetate (150 mL×3). The organic extracts were washed with brine (50 mL), dried over anhydrous Na₂SO₄, filtered, and concentrated. The resulting residue was purified by flash column chromatography (ethyl acetate/heptane) to afford 5-bromo-2-fluoro-4-iodoaniline (10.4 g, 32.9 mmol, 62.6%) as a dark solid.

¹H NMR (CDCl₃, 400 MHz) δ 7.37 (d, J=10.3 Hz, 1H), 7.03 (t, J=8.4 Hz, 1H), 3.79 (s, 2H).

MS (ESI): m/z=313.8 [M−H]⁻.

### Step B: Synthesis of methyl 5-(4-amino-2-bromo-5-fluorophenyl)pent-4-ynoate

A solution of PdCl₂(PPh₃)₂ (0.71 g, 0.1 equiv., 1.0 mmol), 5-bromo-2-fluoro-4-iodoaniline (3.2 g, 1 equiv., 10 mmol), copper (I) iodide (0.19 g, 0.1 equiv., 1.0 mmol), and triethylamine (3.1 g, 3 equiv., 30 mmol) in THF (40 mL) was purged with N₂. Methyl pent-4-ynoate (1.1 g, 1 equiv., 10 mmol) was added, and the resulting mixture was heated at reflux for 3 hours. The reaction mixture was concentrated. The resulting residue was taken up in water (50 mL), extracted with ethyl acetate (50 mL×3), washed with brine (20 mL), dried over anhydrous Na₂SO₄, filtered, and concentrated. The resulting residue was purified by flash column chromatography (ethyl acetate/heptane) to afford methyl 5-(4-amino-2-bromo-5-fluorophenyl)pent-4-ynoate (1.92 g, 6.40 mmol, 63%) as a brown solid.

¹H NMR (CDCl₃, 400 MHz) δ 7.01 (d, J=11.4 Hz, 1H), 6.91 (d, J=8.4 Hz, 1H), 3.85 (s, 2H), 3.70 (s, 3H), 2.75-2.71 (m, 2H), 2.65-2.61 (m, 2H).

MS (ESI): m/z=297.8 [M−H]⁻.

### Step C: Synthesis of methyl 5-(4-amino-2-bromo-5-fluorophenyl)pentanoate

A solution of methyl 5-(4-amino-2-bromo-5-fluorophenyl)pent-4-ynoate (500 mg, 1 equiv., 1.67 mmol) in MeOH (10 mL) was treated with platinum(IV) oxide (100 mg, 0.264 equiv., 440 μmol). The mixture was stirred under a H₂ balloon at room temperature for 3 hours. The reaction mixture was filtered, and the filtrate was concentrated and purified by flash column chromatography (ethyl acetate/heptane) to afford methyl 5-(4-amino-2-bromo-5-fluorophenyl)pentanoate (350 mg, 1.15 mmol, 69.1%) as a light brown oil.

¹H NMR (CDCl₃, 400 MHz) δ 6.93 (d, J=8.5 Hz, 1H), 6.81 (d, J=11.7 Hz, 1H), 3.65-3.63 (m, 4H), 2.58 (t, J=7.6 Hz, 2H), 2.33 (t, J=7.4 Hz, 2H), 1.70-1.63 (m, 2H), 1.60-1.55 (m, 2H).

### Step D: Synthesis of methyl 5-(4-amino-5-fluoro-2-(4,4,5,5-tetramethyl-1,3,2-dioxaborolan-2-yl)phenyl)pentanoate

A solution of Pd(dppf)Cl₂ (0.48 g, 0.1 equiv., 0.66 mmol), methyl 5-(4-amino-2-bromo-5-fluorophenyl)pentanoate (2.0 g, 1 equiv., 6.6 mmol), potassium acetate (1.9 g, 3 equiv., 20 mmol), and 4,4,4′,4′,5,5,5′,5′-octamethyl-2,2′-bi(1,3,2-dioxaborolane) (3.3 g, 2 equiv., 13 mmol) in 1,4-dioxane (30 mL) was degassed with argon. The reaction mixture was warmed at 80° C. for 18 hours. Afterward, the reaction mixture was diluted with water (50 mL), extracted with ethyl acetate (50 mL×3), washed with brine (50 mL), dried over anhydrous Na₂SO₄, filtered and concentrated. The resulting residue was purified by flash column chromatography (ethyl acetate/heptane) to afford methyl 5-(4-amino-5-fluoro-2-(4,4,5,5-tetramethyl-1,3,2-dioxaborolan-2-yl)phenyl)pentanoate (1.89 g, 5.38 mmol, 82%) as a brown oil.

¹H NMR (CDCl₃, 400 MHz) δ 7.22 (d, J=10.1 Hz, 1H), 6.79 (d, J=12.3 Hz, 1H), 3.66 (s, 3H), 3.57 (br s, 2H), 2.77 (t, J=7.7 Hz, 2H), 2.33 (t, J=7.6 Hz, 2H), 1.63-1.71 (m, 2H), 1.47-1.57 (m, 2H), 1.32 (s, 12H).

MS (ESI): m/z=352.3 [M+H]⁺.

### Synthesis of methyl 5-(4-amino-3-fluoro-2-(4,4,5,5-tetramethyl-1,3,2-dioxaborolan-2-yl)phenyl)pentanoate

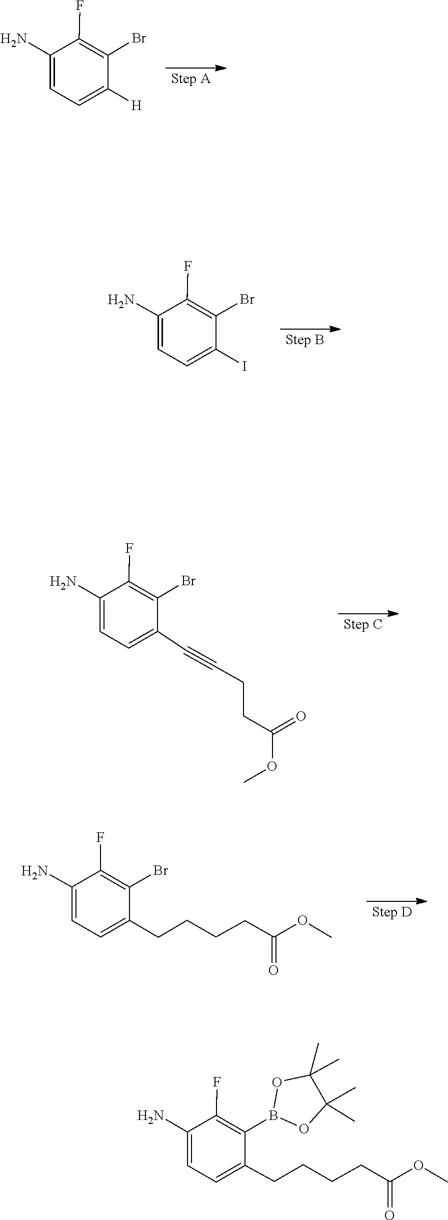 [SMILES: Nc1cccc(Br)c1F ; Nc1ccc(I)c(Br)c1F ; COC(=O)CCC#Cc1ccc(N)c(F)c1Br ; COC(=O)CCCCc1ccc(N)c(F)c1Br ; COC(=O)CCCCc1ccc(N)c(F)c1B1OC(C)(C)C(C)(C)O1]

### Step A: Synthesis of 3-bromo-2-fluoro-4-iodoaniline

NIS (6.2 g, 1.05 equiv., 28 mmol) was added portion wise to a solution of 3-bromo-2-fluoroaniline (5.0 g, 1 equiv., 26 mmol) in MeCN (20 mL) at room temperature. The mixture was stirred for 16 hours. The reaction mixture was concentrated and was purified by flash column chromatography (ethyl acetate/heptane) to afford 3-bromo-2-fluoro-4-iodoaniline (7.5 g, 24 mmol, 90%) as a brown solid.

¹H NMR (CDCl₃, 400 MHz) δ 7.38 (dd, J=8.5, 1.7 Hz, 1H), 6.52 (t, J=8.5 Hz, 1H), 3.85 (s, 2H).

MS (ESI): m/z=315.8 [M+H]⁺.

### Step B: Synthesis of methyl 5-(4-amino-2-bromo-3-fluorophenyl)pent-4-ynoate

A solution of methyl pent-4-ynoate (1.2 g, 1 equiv., 11 mmol), 3-bromo-2-fluoro-4-iodoaniline (3.5 g, 1 equiv., 11 mmol), CuI (0.42 g, 0.20 equiv., 2.2 mmol), and PdCl₂(PPh₃)₂ (0.78 g, 0.10 equiv., 1.1 mmol) in THF (100 mL) under nitrogen was treated with triethylamine (3.4 g, 4.6 mL, 3 equiv., 33 mmol). The reaction mixture was heated at 73° C. for 16 hours. The reaction mixture was concentrated, and the resulting residue was purified by flash column chromatography (ethyl acetate/heptane) to afford methyl 5-(4-amino-2-bromo-3-fluorophenyl)pent-4-ynoate (1.72 g, 5.73 mmol, 52%) as a light brown solid.

¹H NMR (CDCl₃, 400 MHz) δ 7.02 (dd, J=8.4, 1.5 Hz, 1H), 6.61 (t, J=8.4 Hz, 1H), 3.90 (s, 2H), 3.72 (s, 3H), 2.75-2.79 (m, 2H), 2.63-2.67 (m, 2H).

MS (ESI): m/z=300.0 [M+H]⁺.

### Step C: Synthesis of methyl 5-(4-amino-2-bromo-3-fluorophenyl)pentanoate

A solution of methyl 5-(4-amino-2-bromo-3-fluorophenyl)pent-4-ynoate (1.72 g, 1 equiv., 5.73 mmol) in MeOH (20 mL) was treated with PtO₂ (226 mg, 0.15 equiv., 860 μmol) and purged with hydrogen. The reaction mixture was stirred under hydrogen balloon for 16 hours at room temperature. The reaction mixture was filtered, and the filtrate was concentrated and purified by flash column chromatography (ethyl acetate/heptane) to afford the methyl 5-(4-amino-2-bromo-3-fluorophenyl)pentanoate (1.3 g, 4.3 mmol, 75%) as a brown solid.

¹H NMR (CDCl₃, 400 MHz) δ 6.78 (dd, J=8.2, 1.6 Hz, 1H), 6.65 (t, J=8.4 Hz, 1H), 3.69 (s, 2H), 3.67 (s, 3H), 2.66 (t, J=7.6 Hz, 2H), 2.32-2.36 (m, 2H), 1.56-1.71 (m, 4H).

MS (ESI): m/z=304.0 [M+H]⁺.

### Step D: Synthesis of methyl 5-(4-amino-3-fluoro-2-(4,4,5,5-tetramethyl-1,3,2-dioxaborolan-2-yl)phenyl)pentanoate

A solution of bis(pinacolato)diborane (3.01 g, 2 equiv., 11.8 mmol), methyl 5-(4-amino-2-bromo-3-fluorophenyl)pentanoate (1.80 g, 1 equiv., 5.92 mmol), potassium acetate (1.74 g, 3 equiv., 17.8 mmol), and Pd(dppf)Cl₂·CH₂Cl₂ (483 mg, 0.10 equiv., 592 μmol) in t-BuOH (30 mL) was purged with nitrogen and warmed at 90° C. for 20 hours. The reaction mixture was diluted with EtOAc (40 mL) and water (40 mL). The organic layers were separated, and the aqueous layer was extracted with EtOAc (40 mL×3). The combined organic layers were washed with brine (20 mL×3), dried over anhydrous Na₂SO₄, filtered, and concentrated. The resulting residue was purified by flash column chromatography (ethyl acetate/heptane) to afford methyl 5-(4-amino-3-fluoro-2-(4,4,5,5-tetramethyl-1,3,2-dioxaborolan-2-yl)phenyl)pentanoate (1.73 g, 4.93 mmol, 83.2%).

¹H NMR (CDCl₃, 400 MHz) δ 6.69-6.74 (m, 2H), 3.65 (s, 3H), 3.46-3.57 (br m, 2H), 2.62 (t, J=7.7 Hz, 2H), 2.31 (t, J=7.4 Hz, 2H), 1.61-1.68 (m, 2H), 1.50-1.58 (m, 2H), 1.37 (s, 12H).

MS (ESI): m/z=352.2 [M+H]⁺.

### Synthesis of ethyl 4-(2-fluoro-6-(methoxymethoxy)-8-(4,4,5,5-tetramethyl-1,3,2-dioxaborolan-2-yl)naphthalen-1-yl)butanoate

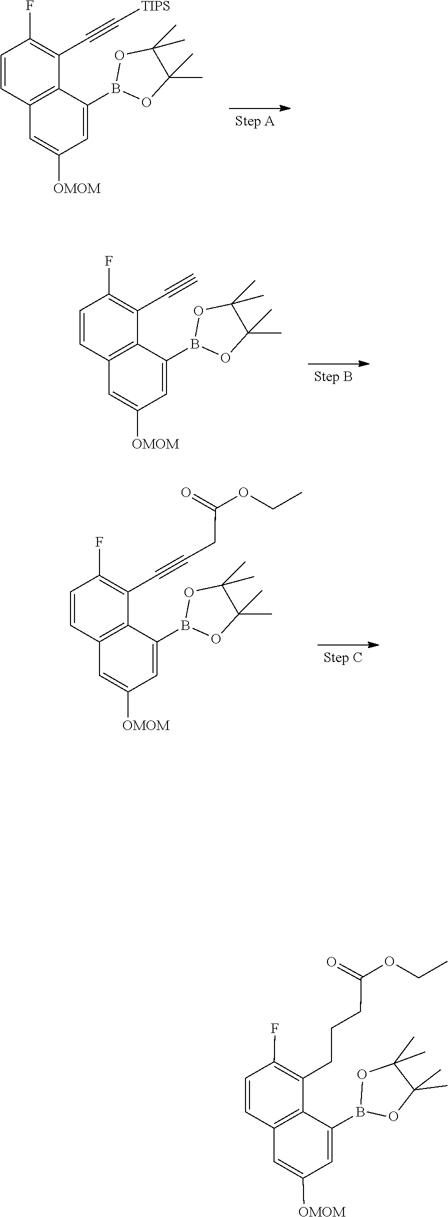

### Step A: Synthesis of 2-(8-ethynyl-7-fluoro-3-(methoxymethoxy)naphthalen-1-yl)-4,4,5,5-tetramethyl-1,3,2-dioxaborolane

A solution of ((2-fluoro-6-(methoxymethoxy)-8-(4,4,5,5-tetramethyl-1,3,2-dioxaborolan-2-yl)naphthalen-1-yl)ethynyl)triisopropylsilane (5000 mg, 1 equiv., 9.76 mmol) in DMSO (50 mL) was treated with cesium fluoride (2.96 g, 2 equiv., 19.5 mmol). The mixture was stirred at room temperature for 1 hour. The resulting residue was diluted with water (100 mL) and extracted with ethyl acetate (100 mL×3). The combined organic layers were washed with brine (80 mL×2), dried over Na₂SO₄, filtered, and concentrated under reduced pressure to give a residue. The residue was purified by flash column chromatography (ethyl acetate/heptane) to afford 2-(8-ethynyl-7-fluoro-3-(methoxymethoxy)naphthalen-1-yl)-4,4,5,5-tetramethyl-1,3,2-dioxaborolane (2.7 g, 7.6 mmol, 78%) as a yellow solid.

MS (ESI): m/z=357.0 [M+H]⁺.

### Step B: Synthesis of ethyl 4-(2-fluoro-6-(methoxymethoxy)-8-(4,4,5,5-tetramethyl-1,3,2-dioxaborolan-2-yl)naphthalen-1-yl)but-3-ynoate

A solution of 2-(8-ethynyl-7-fluoro-3-(methoxymethoxy)naphthalen-1-yl)-4,4,5,5-tetramethyl-1,3,2-dioxaborolane (2700 mg, 1 equiv., 7.58 mmol) and ethyl 2-diazoacetate (951 mg, 1.1 equiv., 8.34 mmol) in MeCN (30 mL) under nitrogen was treated with copper (I) iodide (72.2 mg, 0.05 equiv., 379 μmol). The mixture was stirred at room temperature for 10 hours. The reaction mixture was diluted with water (20 mL) and extracted with ethyl acetate (30 mL×3). The combined organic layers were washed with brine (40 mL×2), dried over Na₂SO₄, filtered, and concentrated under reduced pressure to give a residue. The residue was purified by flash column chromatography (ethyl acetate/heptane) to afford ethyl 4-(2-fluoro-6-(methoxymethoxy)-8-(4,4,5,5-tetramethyl-1,3,2-dioxaborolan-2-yl)naphthalen-1-yl)but-3-ynoate (950 mg, 2.15 mmol, 28.3%) as a yellow oil.

MS (ESI): m/z=443.2 [M+H]⁺.

### Step C: Synthesis of ethyl 4-(2-fluoro-6-(methoxymethoxy)-8-(4,4,5,5-tetramethyl-1,3,2-dioxaborolan-2-yl)naphthalen-1-yl)butanoate

A solution of ethyl 4-(2-fluoro-6-(methoxymethoxy)-8-(4,4,5,5-tetramethyl-1,3,2-dioxaborolan-2-yl)naphthalen-1-yl)but-3-ynoate (1300 mg, 1 equiv., 2.94 mmol) in MeOH (30 mL) was treated with Rh/C (605 mg). The mixture was stirred under a hydrogen atmosphere at room temperature for 24 hours. The reaction mixture was filtered, and the filtrate was concentrated to give a residue. The residue was purified by flash column chromatography (ethyl acetate/heptane) to afford ethyl 4-(2-fluoro-6-(methoxymethoxy)-8-(4,4,5,5-tetramethyl-1,3,2-dioxaborolan-2-yl)naphthalen-1-yl)butanoate (837 mg, 1.88 mmol, 63.8%) as a colorless oil.

¹H NMR (400 MHz, CDCl₃) δ=7.59 (dd, J=5.9, 8.7 Hz, 1H), 7.43-7.39 (m, 2H), 7.24-7.17 (m, 1H), 5.28 (s, 2H), 4.08 (q, J=7.2 Hz, 2H), 3.52 (s, 3H), 3.24 (t, J=6.8 Hz, 2H), 2.28-2.16 (m, 2H), 2.01-1.97 (m, 2H), 1.48-1.40 (m, 12H), 1.21 (t, J=7.2 Hz, 3H).

### Synthesis of diisopropyl (2-(but-3-en-1-yl)-3-chloro-5-(methoxymethoxy)phenyl)boronate

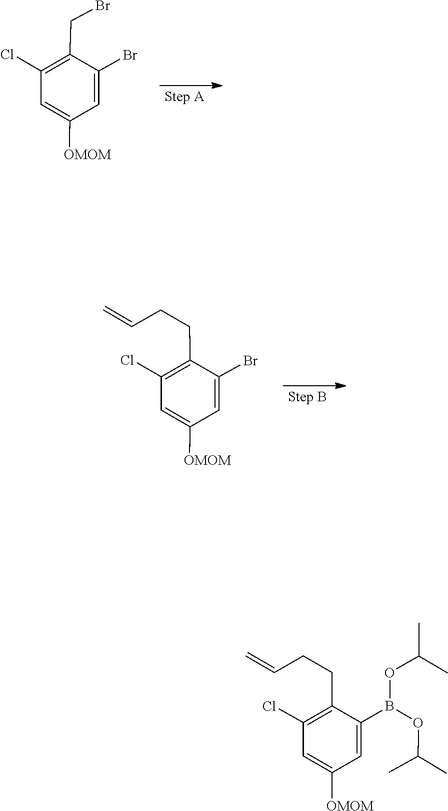

### Step A: Synthesis of 1-bromo-2-(but-3-en-1-yl)-3-chloro-5-(methoxymethoxy)benzene

A solution of 1-bromo-2-(bromomethyl)-3-chloro-5-(methoxymethoxy)benzene (300 mg, 871 μmol) in anhydrous THF (5 mL) under nitrogen at −78° C. was treated dropwise with allylmagnesium bromide in diethyl ether (190 mg, 1.31 mL, 1 M, 1.31 mmol) and the reaction mixture was stirred at −78° C. for 10 minutes then was warmed to room temperature and was stirred for 16 hours. The reaction mixture was quenched with water and was diluted with ethyl acetate, washed with water and brine, dried (Na₂SO₄), filtered, and concentrated. The resulting residue was purified by flash column chromatography (ethyl acetate/heptane) to afford 1-bromo-2-(but-3-en-1-yl)-3-chloro-5-(methoxymethoxy)benzene (167 mg, 546 μmol, 62.7%) as a colorless oil.

¹H NMR (400 MHz, DMSO) δ 7.29 (d, J=2.5 Hz, 1H), 7.18 (d, J=2.5 Hz, 1H), 5.97-5.83 (m, 1H), 5.22 (s, 2H), 5.10-5.03 (m, 1H), 5.03-4.96 (m, 1H), 3.37 (s, 3H), 2.93-2.86 (m, 2H), 2.27-2.19 (m, 2H).

### Step B: Synthesis of diisopropyl (2-(but-3-en-1-yl)-3-chloro-5-(methoxymethoxy)phenyl)boronate

A solution of 1-bromo-2-(but-3-en-1-yl)-3-chloro-5-(methoxymethoxy)benzene (110 mg, 360 μmol) in THF (6 mL) at −78° C. under nitrogen was treated with n-butyllithium in hexanes (46.1 mg, 288 μL, 2.5 molar, 720 μmol) and was stirred at −78° C. for 1 hour. Triisopropyl borate (135 mg, 166 μL, 720 μmol) was added and the reaction mixture was stirred at −78° C. for 2 hours. The reaction mixture was quenched with saturated aqueous ammonium chloride, diluted with ethyl acetate, washed with brine, dried (Na₂SO₄), filtered, and concentrated to afford crude diisopropyl (2-(but-3-en-1-yl)-3-chloro-5-(methoxymethoxy)phenyl)boronate which was used directly in the next step without purification.

### Synthesis of 2-(3-chloro-5-(methoxymethoxy)-2-vinylphenyl)-4,4,5,5-tetramethyl-1,3,2-dioxaborolane

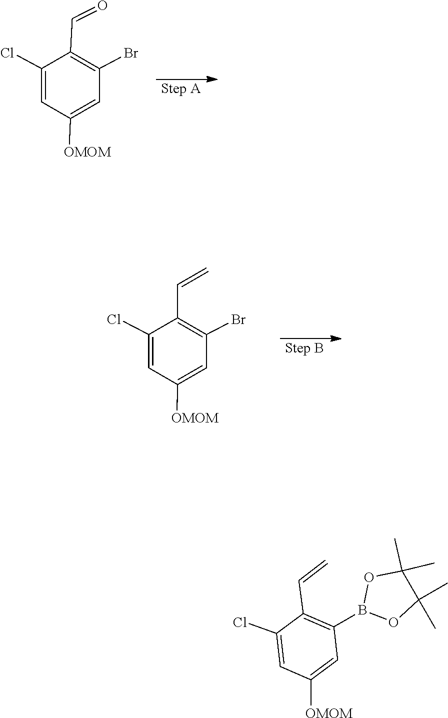

### Step A: Synthesis of 1-bromo-3-chloro-5-(methoxymethoxy)-2-vinylbenzene

A solution of 2-bromo-6-chloro-4-(methoxymethoxy)benzaldehyde (1.5 g, 1 equiv., 5.4 mmol) and methyltriphenylphosphonium bromide (2.3 g, 1.2 equiv., 6.4 mmol) in 1,4-dioxane (20 mL) under nitrogen was treated with potassium carbonate (1.5 g, 2 equiv., 11 mmol). The reaction mixture was warmed at 100° C. for 16 hours. The mixture was concentrated, and the resulting residue was purified by flash column chromatography (ethyl acetate/heptane) to afford 1-bromo-3-chloro-5-(methoxymethoxy)-2-vinylbenzene (0.64 g, 2.3 mmol, 43%) as a colorless oil.

¹H NMR (400 MHz, DMSO) δ 7.34 (d, J=2.5 Hz, 1H), 7.22 (d, J=2.5 Hz, 1H), 6.58 (dd, J=17.8, 11.6 Hz, 1H), 5.66 (dd, J=11.6, 1.4 Hz, 1H), 5.60 (dd, J=17.8, 1.4 Hz, 1H), 5.25 (s, 2H), 3.38 (s, 3H).

### Step B: Synthesis of 2-(3-chloro-5-(methoxymethoxy)-2-vinylphenyl)-4,4,5,5-tetramethyl-1,3,2-dioxaborolane

A solution of 1-bromo-3-chloro-5-(methoxymethoxy)-2-vinylbenzene (0.63 g, 1 equiv., 2.3 mmol), 4,4,4′,4′,5,5,5′,5′-octamethyl-2,2′-bi(1,3,2-dioxaborolane) (0.86 g, 1.5 equiv., 3.4 mmol), PdCl₂(dppf)·CH₂Cl₂ (0.19 g, 0.1 equiv., 0.23 mmol), and potassium acetate (0.67 g, 3 equiv., 6.8 mmol) in 1,4-dioxane (20 mL) was purged with nitrogen and warmed at 80° C. for 16 hours. The reaction mixture was concentrated, and the resulting residue was purified by flash column chromatography (ethyl acetate/heptane) to afford 2-(3-chloro-5-(methoxymethoxy)-2-vinylphenyl)-4,4,5,5-tetramethyl-1,3,2-dioxaborolane (0.47 g, 1.4 mmol, 64%) as a yellow oil.

¹H NMR (400 MHz, DMSO) δ 7.19 (d, J=2.6 Hz, 1H), 7.08 (d, J=2.6 Hz, 1H), 6.91 (dd, J=17.5, 11.1 Hz, 1H), 5.42 (dd, J=11.1, 1.4 Hz, 1H), 5.35 (dd, J=17.5, 1.4 Hz, 1H), 5.22 (s, 2H), 3.37 (s, 3H), 1.27 (s, 12H).

### Synthesis of diisopropyl (3-chloro-2-(hex-5-en-1-yl)-5-(methoxymethoxy)phenyl)boronate

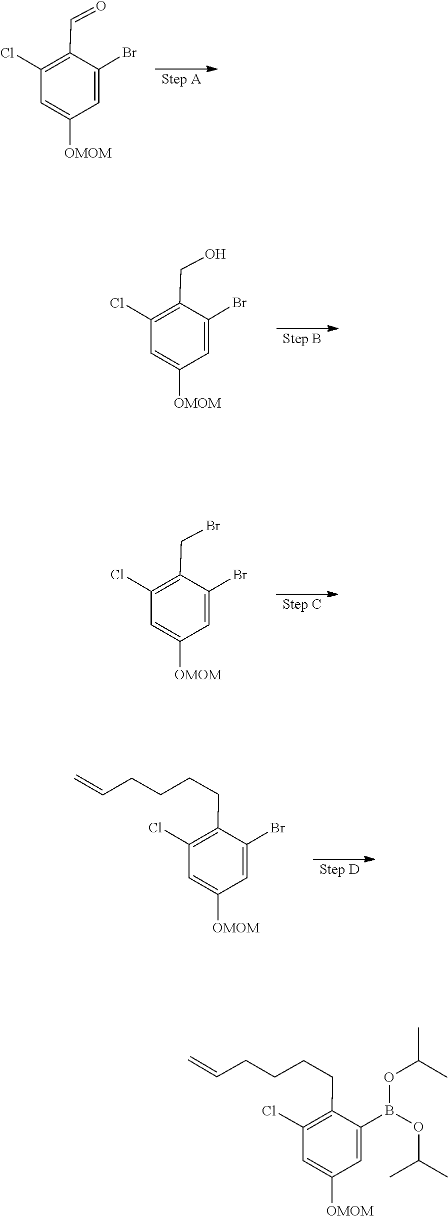

### Step A: Synthesis of (2-bromo-6-chloro-4-(methoxymethoxy)phenyl)methanol

A solution of 2-bromo-6-chloro-4-(methoxymethoxy)benzaldehyde (3.60 g, 1 equiv., 12.9 mmol) in methanol (20 mL) was chilled to 0° C. and treated portion-wise with NaBH₄ (487 mg, 1 equiv., 12.9 mmol). The reaction mixture was stirred at 0° C. for 30 minutes and then allowed to warm to room temperature. After 18 hours, the reaction mixture was diluted with water and extracted into ethyl acetate. The organic layer was further washed with brine, dried over sodium sulfate, filtered, and concentrated onto silica. Purification via flash column chromatography using a gradient of 0-100% B (A: heptane; B: EtOAc) gave (2-bromo-6-chloro-4-(methoxymethoxy)phenyl)methanol (2.54 g, 87% yield).

¹H NMR (400 MHz, CDCl₃) δ 7.22 (d, J=2.4 Hz, 1H), 7.08 (d, J=2.5 Hz, 1H), 5.14 (s, 2H), 4.91 (s, 2H), 3.46 (s, 3H), 1.99 (s, 1H).

### Step B: Synthesis of 1-bromo-2-(bromomethyl)-3-chloro-5-(methoxymethoxy)benzene

A solution of (2-bromo-6-chloro-4-(methoxymethoxy)phenyl)methanol (2.54 g, 1 equiv., 9.02 mmol) and carbon tetrabromide (3.29 g, 1.1 equiv., 9.92 mmol) in DCM (50 mL) was treated with triphenylphosphine (2.60 g, 1.1 equiv., 9.92 mmol). The reaction mixture was stirred at room temperature. After 1 hour, the reaction mixture was concentrated onto silica. Purification via flash column chromatography using a gradient of 0-100% B (A: heptane; B: EtOAc) gave 1-bromo-2-(bromomethyl)-3-chloro-5-(methoxymethoxy)benzene (1.00 g, 31% yield).

¹H NMR (400 MHz, CDCl₃) δ 7.22 (d, J=2.5 Hz, 1H), 7.09 (d, J=2.5 Hz, 1H), 5.14 (s, 2H), 4.77 (s, 2H), 3.47 (s, 3H).

### Step C: Synthesis of 1-bromo-3-chloro-2-(hex-5-en-1-yl)-5-(methoxymethoxy)benzene

A 250 mL 3-neck round-bottom flask was first washed with THF (6.0 mL), equipped with a stirrer bar, and blown dry with a steady flow of nitrogen gas. Magnesium (175 mg, 2.48 equiv., 7.22 mmol) was then added, covered in THF (6.0 mL) under N₂, and chilled to −78° C. Then, 5-bromopent-1-ene (730 mg, 1.68 equiv., 4.90 mmol) was added. After 30 minutes, the reaction mixture was allowed to warm to room temperature. After 16 hours, the reaction mixture was warmed to 45° C. for 1 hour until most of the magnesium was dissolved and then cooled to room temperature. The Grignard solution was then added dropwise under nitrogen to a solution of 1-bromo-2-(bromomethyl)-3-chloro-5-(methoxymethoxy)benzene (1.00 g, 1 equiv., 2.91 mmol) in THF (6.0 mL) at −78° C. Following the addition, the reaction mixture was allowed to warm to room temperature. After 3 hours, the reaction mixture was diluted with ethyl acetate, washed with saturated aqueous NH₄Cl and brine, dried (Na₂SO₄), filtered, and concentrated onto celite. Purification via reverse phase flash column chromatography using a gradient of 0-100% B (A: water+0.1% formic acid; B: acetonitrile+0.1% formic acid) provided 1-bromo-3-chloro-2-(hex-5-en-1-yl)-5-(methoxymethoxy)benzene (206 mg, 21% yield).

¹H NMR (400 MHz, CDCl₃) δ 7.18 (d, J=2.5 Hz, 1H), 7.04 (d, J=2.5 Hz, 1H), 5.91-5.76 (m, 1H), 5.11 (s, 2H), 5.07-4.97 (m, 1H), 5.00-4.91 (m, 1H), 3.46 (s, 3H), 2.91-2.82 (m, 2H), 2.17-2.06 (m, 2H), 1.62-1.45 (m, 4H).

### Step D: Synthesis of diisopropyl (3-chloro-2-(hex-5-en-1-yl)-5-(methoxymethoxy)phenyl)boronate

1-Bromo-3-chloro-2-(hex-5-en-1-yl)-5-(methoxymethoxy)benzene (157 mg, 1.0 equiv., 469 μmol) was dissolved in THF (8.00 mL). The mixture was chilled to −78° C. followed by the addition of N-butyllithium (60.1 mg, 375 μL, 2.5 M, 2.0 equiv., 938 μmol). The reaction mixture was stirred at −78° C. for 1 hour. Triisopropyl borate (176 mg, 128 μL, 2.0 equiv., 938 μmol) was added, and the reaction mixture was stirred at −78° C. for 2 hours. The reaction mixture was quenched by addition of saturated aqueous NH₄Cl (2 mL). The reaction mixture was diluted with EtOAc, washed with water and brine, dried (Na₂SO₄), filtered, and evaporated to dryness. The crude residue was used in the following step without purification.

¹H NMR (400 MHz, CDCl₃) δ 7.15-7.10 (m, 1H), 7.08-7.03 (m, 1H), 5.88-5.74 (m, 1H), 5.18 (s, 1H), 5.14 (s, 2H), 5.05-4.91 (m, 3H), 3.49 (s, 2H), 2.89-2.81 (m, 2H), 2.13-2.06 (m, 2H), 2.04 (s, 4H), 1.43 (s, 2H), 1.41-1.30 (m, 3H), 0.94-0.87 (m, 4H), 0.87-0.77 (m, 4H).

### Synthesis of methyl 5-(4-amino-2-chloro-3-fluoro-6-(4,4,5,5-tetramethyl-1,3,2-dioxaborolan-2-yl)phenyl)pentanoate

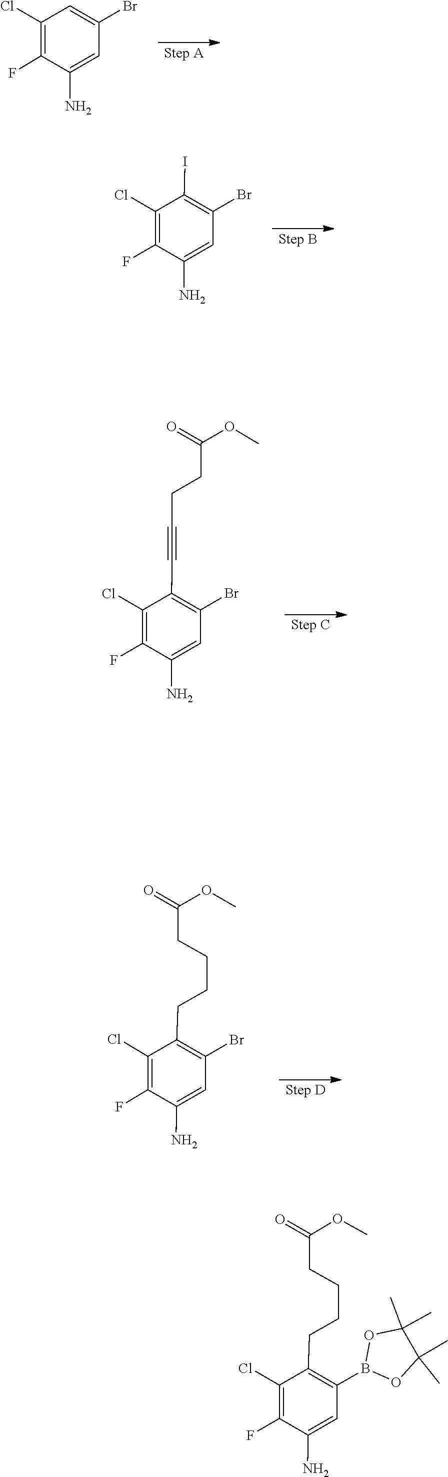 [SMILES: Nc1cc(Br)cc(Cl)c1F ; Nc1cc(Br)c(I)c(Cl)c1F ; COC(=O)CCC#Cc1c(Br)cc(N)c(F)c1Cl ; COC(=O)CCCCc1c(Br)cc(N)c(F)c1Cl ; COC(=O)CCCCc1c(B2OC(C)(C)C(C)(C)O2)cc(N)c(F)c1Cl]

### Step A: Synthesis of 5-bromo-3-chloro-2-fluoro-4-iodoaniline

A mixture of 5-bromo-3-chloro-2-fluoroaniline (43.3 g, 193 mmol) and NIS (43.4 g, 193 mmol) in AcOH (200 mL) was degassed and purged with N₂ three times. The mixture was stirred at 40° C. for 1 hour. The reaction mixture was quenched by saturated aqueous NaHCO₃ (400 mL), diluted with H₂O (200 mL), extracted with ethyl acetate (300 mL×3), dried over Na₂SO₄, filtered, and concentrated under reduced pressure to give a residue. The residue was purified by column chromatography (petroleum ether/ethyl acetate=100/1 to 95/5) to afford 5-bromo-3-chloro-2-fluoro-4-iodoaniline (17.5 g, 45 mmol, 23%) as a yellow solid.

MS (ESI): m/z=349.9 [M+H]⁺.

### Step B: Synthesis of methyl 5-(4-amino-6-bromo-2-chloro-3-fluorophenyl)pent-4-ynoate

A mixture of 5-bromo-3-chloro-2-fluoro-4-iodoaniline (15.5 g, 44.2 mmol), Pd(dppf)Cl₂ (9.71 g, 13.3 mmol), CuI (2.53 g, 13.3 mmol), and methyl pent-4-ynoate (14.9 g, 133 mmol) in triethylamine (200 mL) was degassed and purged with N₂ three times. The mixture was stirred at 100° C. for 12 hours. The reaction mixture was diluted with H₂O (300 mL), extracted with ethyl acetate (300 mL×3), dried over Na₂SO₄, filtered, and concentrated under reduced pressure to give a residue. The residue was purified by column chromatography (petroleum ether/ethyl acetate=100/1 to 85/15) to afford methyl 5-(4-amino-6-bromo-2-chloro-3-fluorophenyl)pent-4-ynoate (4.0 g, 10 mmol, 24%) as a white solid.

MS (ESI): m/z=334.1 [M+H]⁺.

### Step C: Synthesis of methyl 5-(4-amino-6-bromo-2-chloro-3-fluorophenyl)pentanoate

A mixture of PtO₂ (2.7 g, 12 mmol) and methyl 5-(4-amino-6-bromo-2-chloro-3-fluorophenyl)pent-4-ynoate (4.0 g, 12 mmol) in MeOH (20 mL) was degassed and purged with N₂ three times. The mixture was stirred at 25° C. for 2 hours under H₂ atmosphere. The reaction mixture was filtered and concentrated under reduced pressure to give a residue. The residue was purified by column chromatography (petroleum ether/ethyl acetate=100/1 to 92/8) to afford methyl 5-(4-amino-6-bromo-2-chloro-3-fluorophenyl)pentanoate (2.0 g, 5.7 mmol, 48%) as a yellow oil.

MS (ESI): m/z=338.0 [M+H]⁺.

### Step D: Synthesis of methyl 5-(4-amino-2-chloro-3-fluoro-6-(4,4,5,5-tetramethyl-1,3,2-dioxaborolan-2-yl)phenyl)pentanoate

A mixture of methyl 5-(4-amino-6-bromo-2-chloro-3-fluorophenyl)pentanoate (2.00 g, 5.91 mmol), potassium acetate (1.74 g, 17.7 mmol), bis(pinacolato)diborane (3.00 g, 11.8 mmol), and Pd(dppf)Cl₂·CH₂Cl₂ (482 mg, 591 μmol) in 1,4-dioxane (20 mL) was degassed and purged with N₂ three times. The mixture was stirred at 85° C. for 3 hours. The reaction mixture was diluted with H₂O (50 mL), extracted with ethyl acetate (50 mL×3), dried over Na₂SO₄, filtered, and concentrated under reduced pressure to give a residue. The residue was purified by column chromatography (petroleum ether/ethyl acetate=100/1 to 96/4) to afford methyl 5-(4-amino-2-chloro-3-fluoro-6-(4,4,5,5-tetramethyl-1,3,2-dioxaborolan-2-yl)phenyl)pentanoate (1.72 g, 4.09 mmol, 69.3%) as a yellow oil.

MS (ESI): m/z=386.2 [M+H]⁺.

¹H NMR (400 MHz, DMSO-d₆) δ 7.11 (d, J=9.2 Hz, 1H), 5.25 (s, 2H), 3.56 (s, 3H), 2.89-2.79 (m, 2H), 2.33-2.29 (m, 2H), 1.62-1.53 (m, 2H), 1.44-1.36 (m, 2H), 1.28 (s, 12H)

### Synthesis of methyl 5-(4-amino-2-(4,4,5,5-tetramethyl-1,3,2-dioxaborolan-2-yl)-6-(trifluoromethyl)phenyl)pentanoate

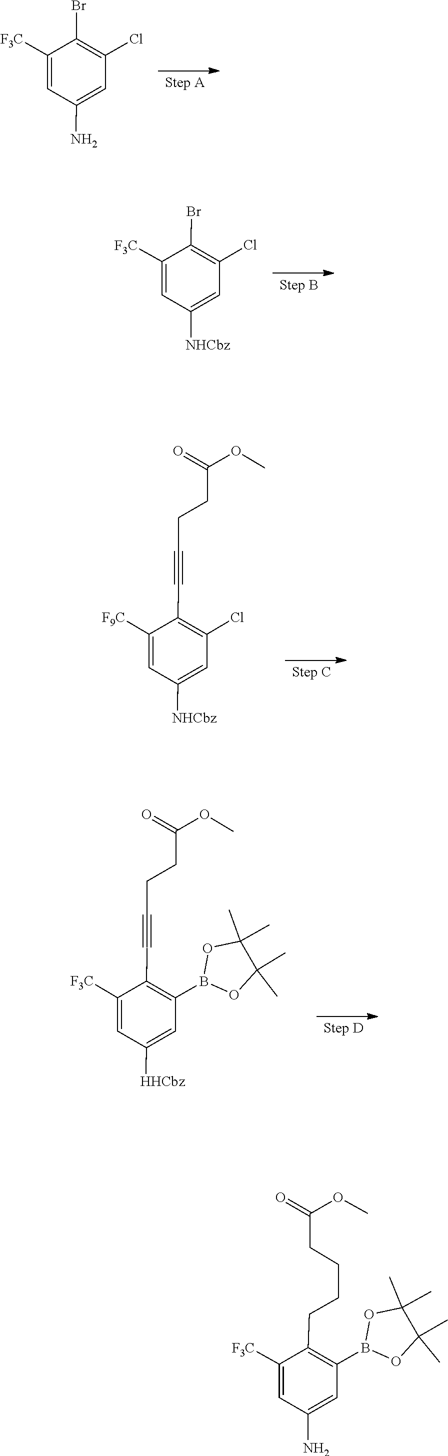

### Step A: Synthesis of benzyl (4-bromo-3-chloro-5-(trifluoromethyl)phenyl)carbamate

A solution of 4-bromo-3-chloro-5-(trifluoromethyl) aniline (13.0 g, 47.4 mmol) and DIPEA (18.4 g, 24.8 mL, 142 mmol) in DCM (100 mL) at 0° C. was treated with Cbz-Cl (16.16 g, 13.5 mL, 94.7 mmol). The reaction mixture was stirred at room temperature for 16 hours. The reaction mixture was diluted with H₂O and ethyl acetate, washed with brine, dried (Na₂SO₄), filtered, and concentrated under reduced pressure to give a residue. The residue was purified by column chromatography (petroleum ether/ethyl acetate=I/O to 5/1) to afford benzyl (4-bromo-3-chloro-5-(trifluoromethyl)phenyl)carbamate (15.0 g, 31 mmol, 66%) as a white solid.

MS (ESI): m/z=408.1 [M+H]⁺.

### Step B: Synthesis of methyl 5-(4-(((benzyloxy)carbonyl)amino)-2-chloro-6-(trifluoromethyl)phenyl)pent-4-ynoate

A solution of benzyl (4-bromo-3-chloro-5-(trifluoromethyl)phenyl)carbamate (15.0 g, 36.7 mmol), methyl pent-4-ynoate (4.53 g, 40.4 mmol) and Cs₂CO₃ (23.9 g, 73.4 mmol) in THE (90 mL) and MeCN (90 mL) was degassed and purged with N₂ three times. XPhos-Pd-G3 (3.11 g, 3.67 mmol) was added, and the reaction mixture was stirred at 60° C. for 2 hours. The reaction mixture was diluted with water and ethyl acetate, washed with brine, dried (Na₂SO₄), filtered, and concentrated under reduced pressure to give a residue. The residue was purified by column chromatography (petroleum ether/ethyl acetate=1/0 to 5/1) to afford methyl 5-(4-(((benzyloxy)carbonyl)amino)-2-chloro-6-(trifluoromethyl)phenyl)pent-4-ynoate (5.4 g, 11 mmol, 29%) as a white solid.

MS (ESI): m/z=440.2 [M+H]⁺.

### Step C: Synthesis of methyl 5-(4-(((benzyloxy)carbonyl)amino)-2-(4,4,5,5-tetramethyl-1,3,2-dioxaborolan-2-yl)-6-(trifluoromethyl)phenyl)pent-4-ynoate

A solution of methyl 5-(4-(((benzyloxy)carbonyl)amino)-2-chloro-6-(trifluoromethyl)phenyl)pent-4-ynoate (6.00 g, 13.6 mmol), XPhos (1.30 g, 2.73 mmol), bis(pinacolato)diboron (8.66 g, 34.1 mmol), and potassium acetate (4.02 g, 40.9 mmol) in 1,4-dioxane (60 mL) was degassed and purged with N₂ three times. Pd₂dba₃ (1.25 g, 1.36 mmol) was added, and the reaction mixture was stirred at 90° C. for 1 hour. The reaction mixture was diluted with water and ethyl acetate, washed with brine, dried (Na₂SO₄), filtered, and concentrated under reduced pressure to give a residue. The residue was purified by column chromatography (petroleum ether/ethyl acetate=1/0 to 5/1) to afford methyl 5-(4-(((benzyloxy)carbonyl)amino)-2-(4,4,5,5-tetramethyl-1,3,2-dioxaborolan-2-yl)-6-(trifluoromethyl)phenyl)pent-4-ynoate (2.5 g, 4.5 mmol, 33%) as a white solid.

MS (ESI): m/z=532.2 [M+H]⁺.

### Step D: Synthesis of methyl 5-(4-amino-2-(4,4,5,5-tetramethyl-1,3,2-dioxaborolan-2-yl)-6-(trifluoromethyl)phenyl)pentanoate

A solution of methyl 5-(4-(((benzyloxy)carbonyl)amino)-2-(4,4,5,5-tetramethyl-1,3,2-dioxaborolan-2-yl)-6-(trifluoromethyl)phenyl)pent-4-ynoate (2.00 g, 3.75 mmol) in THF (30 mL) was degassed and purged with N₂ three times. To this solution was added Pd/C (853 mg, 8.02 mmol) and Pd(OH)₂ (844 mg, 6.01 mmol). The reaction mixture was purged with H₂ three times. The mixture was stirred at room temperature for 96 hours under H₂ atmosphere. The reaction mixture was filtered through celite and was concentrated under reduced pressure to give a residue. The residue was purified by column chromatography (petroleum ether/ethyl acetate=1/0 to 3/1) to afford methyl 5-(4-amino-2-(4,4,5,5-tetramethyl-1,3,2-dioxaborolan-2-yl)-6-(trifluoromethyl)phenyl)pentanoate (1.49 g, 3.7 mmol, 73%) as a white solid.

MS (ESI): m/z=402.2 [M+H]⁺.

### Synthesis of methyl 5-(6-(bis(4-methoxybenzyl)amino)-2-chloro-4-(4,4,5,5-tetramethyl-1,3,2-dioxaborolan-2-yl)pyridin-3-yl)pentanoate

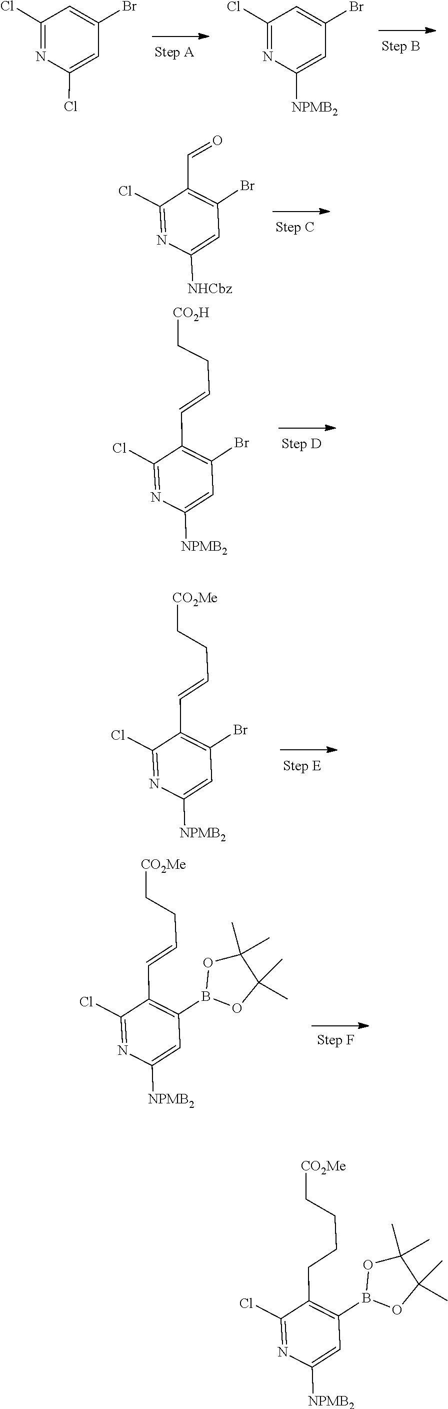

### Step A: Synthesis of 4-bromo-6-chloro-N,N-bis(4-methoxybenzyl)pyridin-2-amine

A solution of 4-bromo-2,6-dichloropyridine (50.0 g, 220 mmol) in DMSO (500 mL) was treated with bis(4-methoxybenzyl)amine (62.4 g, 242 mmol), potassium fluoride (12.8 g, 220 mmol), and DIPEA (85.5 g, 661 mmol). The reaction mixture was stirred at 120° C. for 48 hours. The reaction mixture was diluted with water and ethyl acetate, washed with brine, dried (Na₂SO₄), filtered, and concentrated under reduced pressure to give a residue. The residue was purified by column chromatography (petroleum ether/ethyl acetate=100/1 to 8/1) to afford 4-bromo-6-chloro-N,N-bis(4-methoxybenzyl)pyridin-2-amine (31 g, 65 mmol, 30%) as a yellow solid.

MS (ESI): m/z=447.0 [M+H]⁺.

### Step B: Synthesis of 6-(bis(4-methoxybenzyl)amino)-4-bromo-2-chloronicotinaldehyde

A solution of 4-bromo-6-chloro-N,N-bis(4-methoxybenzyl)pyridin-2-amine (24.0 g, 53.6 mmol) in THF (240 mL) at −78° C. was treated dropwise with LDA in THF/heptane/ethylbenzene (8.61 g, 40.2 mL, 2.0 M, 80.4 mmol). The mixture was stirred at this temperature for 30 min. DMF (19.6 g, 20.8 mL, 268 mmol) was added dropwise at −78° C., and the reaction mixture was stirred at −78° C. for 1 hour. The reaction mixture was quenched by the addition of saturated aqueous NH₄Cl (200 mL) at −78° C. and diluted with DCM. The organic layer was separated, dried (Na₂SO₄), filtered, and concentrated under reduced pressure to give a residue. The residue was purified by column chromatography (petroleum ether/DCM=100/1 to 100/1) to afford 6-(bis(4-methoxybenzyl)amino)-4-bromo-2-chloronicotinaldehyde (20 g, 42 mmol, 78%) as a yellow solid.

¹H NMR (400 MHz, CDCl₃) δ 10.24 (s, 1H), 7.15 (d, J=8.0 Hz, 4H), 6.92-6.85 (m, 4H), 6.69 (s, 1H), 4.90-4.50 (m, 4H), 3.85-3.78 (m, 6H).

### Step C: Synthesis of (E)-5-(6-(bis(4-methoxybenzyl)amino)-4-bromo-2-chloropyridin-3-yl)pent-4-enoic acid

A solution of 6-(bis(4-methoxybenzyl)amino)-4-bromo-2-chloronicotinaldehyde (14.4 g, 30.3 mmol) and (3-carboxypropyl)triphenylphosphonium bromide (14.3 g, 33.3 mmol) in dry THF (140 mL) at 0° C. was treated dropwise with KO^{t}Bu in THF (7.47 g, 66.6 mL, 1.0 M, 66.6 mmol) over 30 min. The reaction mixture was allowed to warm to room temperature. The mixture was stirred for 12 hours. The reaction mixture was diluted with water (100 mL) and HCl (1 M, 15 mL) was added to adjust the pH to 3. The reaction mixture was diluted with EtOAc, washed with brine, dried (Na₂SO₄), filtered and concentrated under reduced pressure to afford crude product (E)-5-(6-(bis(4-methoxybenzyl)amino)-4-bromo-2-chloropyridin-3-yl)pent-4-enoic acid (11.5 g, 21 mmol, 68%) which was used into the next step without further purification.

MS (ESI): m/z=545.0 [M+H]⁺.

### Step D: Synthesis of methyl (E)-5-(6-(bis(4-methoxybenzyl)amino)-4-bromo-2-chloropyridin-3-yl)pent-4-enoate

A solution of (E)-5-(6-(bis(4-methoxybenzyl)amino)-4-bromo-2-chloropyridin-3-yl)pent-4-enoic acid (12.6 g, 23.1 mmol) and Cs₂CO₃ (9.03 g, 27.7 mmol) in acetone (130 mL) was treated with Mel (7.21 g, 3.18 mL, 50.8 mmol). The resulting mixture was stirred for 1 hour at 60° C. under N₂ atmosphere. The reaction mixture was concentrated under reduced pressure to give a residue. The residue was purified by column chromatography (petroleum ether/ethyl acetate=100/1 to 15/1) to afford methyl (E)-5-(6-(bis(4-methoxybenzyl)amino)-4-bromo-2-chloropyridin-3-yl)pent-4-enoate (12 g, 21 mmol, 92%) as a yellow oil.

MS (ESI): m/z=559.1 [M+H]⁺.

### Step E: Synthesis of methyl (E)-5-(6-(bis(4-methoxybenzyl)amino)-2-chloro-4-(4,4,5,5-tetramethyl-1,3,2-dioxaborolan-2-yl)pyridin-3-yl)pent-4-enoate

A mixture of methyl (E)-5-(6-(bis(4-methoxybenzyl)amino)-4-bromo-2-chloropyridin-3-yl)pent-4-enoate (12.0 g, 21.4 mmol), Potassium acetate (7.36 g, 4.69 mL, 75.0 mmol), Bis(pinacolato)diborane (6.53 g, 25.7 mmol) and Pd(dppf)Cl₂·CH₂Cl₂ (1.75 g, 2.14 mmol) in 1,4-dioxane (120 mL) was degassed and purged with N₂ three times. The mixture was stirred at 80° C. for 2 hours under N₂ atmosphere. The reaction mixture was filtered through celite and concentrated under reduced pressure to give a residue. The residue was purified by column chromatography (petroleum ether/ethyl acetate=100/1 to 15/1) to afford methyl (E)-5-(6-(bis(4-methoxybenzyl)amino)-2-chloro-4-(4,4,5,5-tetramethyl-1,3,2-dioxaborolan-2-yl)pyridin-3-yl)pent-4-enoate (8.6 g, 14 mmol, 63%) as a white solid.

MS (ESI): m/z=607.3 [M+H]⁺.

### Step F: Synthesis of methyl 5-(6-(bis(4-methoxybenzyl)amino)-2-chloro-4-(4,4,5,5-tetramethyl-1,3,2-dioxaborolan-2-yl)pyridin-3-yl)pentanoate

A solution of methyl (E)-5-(6-(bis(4-methoxybenzyl)amino)-2-chloro-4-(4,4,5,5-tetramethyl-1,3,2-dioxaborolan-2-yl)pyridin-3-yl)pent-4-enoate (9.2 g, 15 mmol) in MeOH (100 mL) under nitrogen was treated with rhodium on activated carbon (5 g, 5 wt %, 2 mmol) and the suspension was degassed and purged with H₂ three times. The reaction mixture was stirred under H₂ (15 psi) at room temperature for 4 hours. The reaction mixture was filtered and concentrated and the resulting residue was purified by column chromatography (petroleum ether/ethyl acetate=100/1 to 20/1) to afford methyl 5-(6-(bis(4-methoxybenzyl)amino)-2-chloro-4-(4,4,5,5-tetramethyl-1,3,2-dioxaborolan-2-yl)pyridin-3-yl)pentanoate (4 g, 6 mmol, 40%) as a colorless oil.

MS (ESI): m/z=609.4 [M+H]⁺.

### Synthesis of tert-butyl 5-(6-chloro-1-(tetrahydro-2H-pyran-2-yl)-4-(4,4,5,5-tetramethyl-1,3,2-dioxaborolan-2-yl)-1H-indazol-5-yl)pentanoate

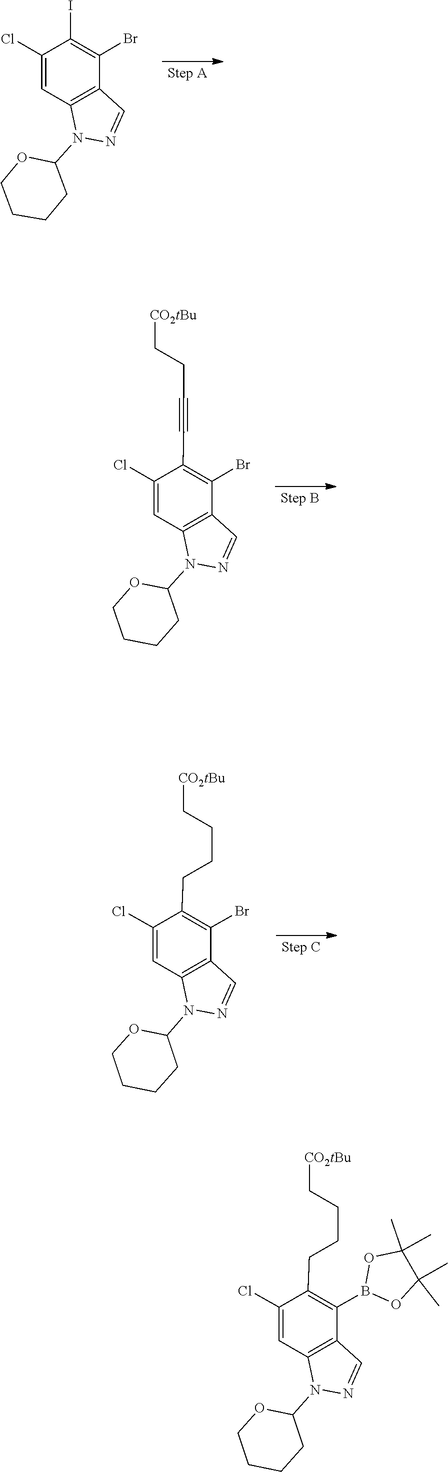 [SMILES: Clc1cc2c(cnn2C2CCCCO2)c(Br)c1I ; CC(C)(C)OC(=O)CCC#Cc1c(Cl)cc2c(cnn2C2CCCCO2)c1Br ; CC(C)(C)OC(=O)CCCCc1c(Cl)cc2c(cnn2C2CCCCO2)c1Br ; CC(C)(C)OC(=O)CCCCc1c(Cl)cc2c(cnn2C2CCCCO2)c1B1OC(C)(C)C(C)(C)O1]

### Step A: Synthesis of tert-butyl 5-(4-bromo-6-chloro-1-(tetrahydro-2H-pyran-2-yl)-1H-indazol-5-yl)pent-4-ynoate

A solution of 4-bromo-6-chloro-5-iodo-1-(tetrahydro-2H-pyran-2-yl)-1H-indazole (5.3 g, 12 mmol), tert-butyl pent-4-ynoate (3.7 g, 24 mmol), and CuI (0.69 g, 3.6 mmol) in Et₃N (50 mL) under nitrogen was treated with Pd(PPh₃)₂Cl₂ (0.84 g, 1.2 mmol) and warmed at 60° C. for 16 hours. The reaction mixture was filtered and concentrated. The resulting residue was purified by flash column chromatography (ethyl acetate/heptane) to afford tert-butyl 5-(4-bromo-6-chloro-1-(tetrahydro-2H-pyran-2-yl)-1H-indazol-5-yl)pent-4-ynoate (4 g, 9 mmol, 70%) as a colorless oil.

¹H NMR (400 MHz, CDCl₃-d) δ 7.95 (s, 1H), 7.64 (d, J=0.8 Hz, 1H), 5.63-5.60 (m, 1H), 4.04-3.94 (m, 1H), 3.78-3.68 (m, 1H), 2.86-2.79 m, 2H), 2.69-2.60 (m, 2H), 2.52-2.41 (m, 1H), 2.14-2.03 (m, 2H), 1.77-1.65 (m, 3H), 1.47 (s, 9H).

### Step B: Synthesis of tert-butyl 5-(4-bromo-6-chloro-1-(tetrahydro-2H-pyran-2-yl)-1H-indazol-5-yl)pentanoate

A solution of tert-butyl 5-(4-bromo-6-chloro-1-(tetrahydro-2H-pyran-2-yl)-1H-indazol-5-yl)pent-4-ynoate (6.5 g, 14 mmol) in MeOH (60 mL) was treated with PtO₂ (0.63 g, 2.8 mmol). The reaction mixture was stirred at room temperature for 12 hours under H₂ atmosphere (15 psi). The mixture was filtered, washed with MeOH, and concentrated under reduced pressure to give a residue. The residue was purified by flash column chromatography (ethyl acetate/heptane) to afford tert-butyl 5-(4-bromo-6-chloro-1-(tetrahydro-2H-pyran-2-yl)-1H-indazol-5-yl)pentanoate (2.5 g, 5.3 mmol, 38%) as a yellow solid.

¹H NMR (400 MHz, CDCl₃-d) δ 7.94 (s, 1H), 7.62 (s, 1H), 5.63-5.61 (m, 1H), 4.06-3.97 (m, 1H), 3.78-3.64 (m, 1H), 3.12-2.98 (m, 2H), 2.48-2.46 (m, 1H), 2.30 (t, J=7.2 Hz, 2H), 2.13-2.05 (m, 2H), 1.78-1.70 (m, 4H), 1.68-1.59 (m, 3H), 1.44 (s, 9H).

### Step C: Synthesis of tert-butyl 5-(6-chloro-1-(tetrahydro-2H-pyran-2-yl)-4-(4,4,5,5-tetramethyl-1,3,2-dioxaborolan-2-yl)-1H-indazol-5-yl)pentanoate

A solution of tert-butyl 5-(4-bromo-6-chloro-1-(tetrahydro-2H-pyran-2-yl)-1H-indazol-5-yl)pentanoate (3.8 g, 8.1 mmol), potassium acetate (2.4 g, 24 mmol), and bis(pinacolato)diborane (4.1 g, 16 mmol) in 1,4-dioxane (40 mL) under nitrogen was treated with PdCl₂(dppf)·CH₂Cl₂ (0.66 g, 0.81 mmol). The reaction mixture was stirred at 100° C. for 16 hours. The reaction mixture was diluted with water, extracted with ethyl acetate, washed with brine, dried (Na₂SO₄), filtered, and concentrated under reduced pressure to give a residue. The resulting residue was purified by flash column chromatography (ethyl acetate/heptane) to afford tert-butyl 5-(6-chloro-1-(tetrahydro-2H-pyran-2-yl)-4-(4,4,5,5-tetramethyl-1,3,2-dioxaborolan-2-yl)-1H-indazol-5-yl)pentanoate (3.5 g, 3.8 mmol, 48%) as a colorless oil which was used as is without further purification.

MS (ESI): m/z=519.3 [M+H]⁺.

### Synthesis of ethyl 5-(1-(tetrahydro-2H-pyran-2-yl)-4-(4,4,5,5-tetramethyl-1,3,2-dioxaborolan-2-yl)-1H-indazol-5-yl)pentanoate

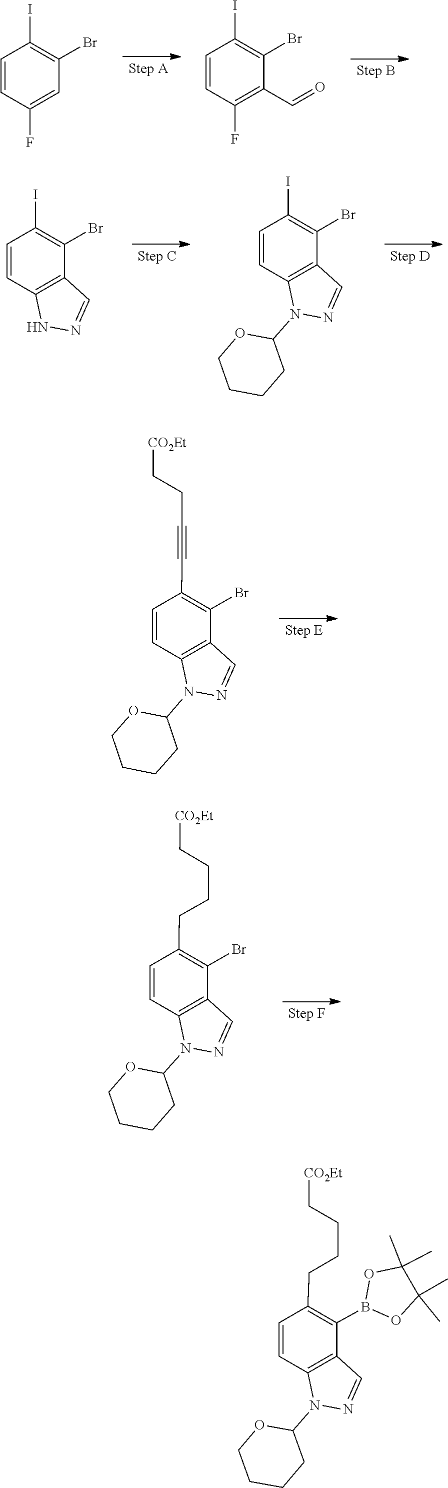

### Step A: Synthesis of 2-bromo-6-fluoro-3-iodobenzaldehyde

A solution of 2-bromo-4-fluoro-1-iodobenzene (500 mg, 1.66 mmol) in THF (5 mL) at −78° C. was treated dropwise with LDA (267 mg, 2.49 mL, 1.0 M, 2.49 mmol). The mixture was stirred for 30 minutes. DMF (182 mg, 193 μL, 2.49 mmol) was added and the reaction was stirred at −78° C. for 1 hour. Saturated aqueous NH₄Cl was added. The reaction mixture was diluted with EtOAc, washed with brine, dried (Na₂SO₄), filtered, and concentrated. The resulting residue was purified by flash column chromatography (ethyl acetate/heptane) to afford 2-bromo-6-fluoro-3-iodobenzaldehyde (331 mg, 1.01 mmol, 60.6%).

¹H NMR (400 MHz, CDCl₃) δ 10.21 (s, 1H), 8.04 (dd, J=8.8, 5.5 Hz, 1H), 6.96 (t, J=9.2 Hz, 1H).

### Step B: Synthesis of 4-bromo-5-iodo-1H-indazole

A solution of 2-bromo-6-fluoro-3-iodobenzaldehyde (330 mg, 1.00 mmol) in DME (8 mL) was treated with N₂H₄·H₂O (502 mg, 487 μL, 10.0 mmol) and warmed at 95° C. for 6 hours. The reaction mixture was concentrated to afford 4-bromo-5-iodo-1H-indazole (324 mg, 1.00 mmol, 100%), which was used directly in the next step without purification. MS (ESI): m/z (+)=322.8.

### Step C: Synthesis of 4-bromo-5-iodo-1-(tetrahydro-2H-pyran-2-yl)-1H-indazole

A solution of 4-bromo-5-iodo-1H-indazole (1580 mg, 4.89 mmol) in THF (8 mL) was treated with PPTS (123 mg, 489 μmol) and 3,4-dihydro-2H-pyran (1.24 g, 1.34 mL, 14.7 mmol). The mixture was stirred at 60° C. for 5 hours. The reaction mixture was concentrated and purified by flash column chromatography (ethyl acetate/heptane) to afford 4-bromo-5-iodo-1-(tetrahydro-2H-pyran-2-yl)-1H-indazole (1.21 g, 2.97 mmol, 60.8%).

MS (ESI): m/z (+)=406.9.

### Step D: Synthesis of ethyl 5-(4-bromo-1-(tetrahydro-2H-pyran-2-yl)-1H-indazol-5-yl)pent-4-ynoate

A solution of 4-bromo-5-iodo-1-(tetrahydro-2H-pyran-2-yl)-1H-indazole (1100 mg, 2.70 mmol), ethyl pent-4-ynoate (375 mg, 2.97 mmol), CuI (51.5 mg, 270 μmol), Et₃N (820 mg, 1.13 mL, 8.11 mmol), and Pd(dppf)Cl₂·CH₂Cl₂ (221 mg, 270 μmol) in MeCN (20 mL) was degassed with nitrogen and warmed at 60° C. for 4 hours. The reaction mixture was concentrated and purified by flash column chromatography to (ethyl acetate/heptane) afford ethyl 5-(4-bromo-1-(tetrahydro-2H-pyran-2-yl)-1H-indazol-5-yl)pent-4-ynoate (716 mg, 1.77 mmol, 65.4%).

MS (ESI): m/z (+)=405.2.

### Step E: Synthesis of ethyl 5-(4-bromo-1-(tetrahydro-2H-pyran-2-yl)-1H-indazol-5-yl)pentanoate

A solution of ethyl 5-(4-bromo-1-(tetrahydro-2H-pyran-2-yl)-1H-indazol-5-yl)pent-4-ynoate (716 mg, 1.77 mmol) and PtO₂ (80.2 mg, 353 μmol) in MeOH (30 mL) was bubbled with hydrogen for 20 minutes. The mixture was stirred under a hydrogen atmosphere for 1 hour. The reaction mixture was filtered and concentrated and purified by flash column chromatography (ethyl acetate/heptane) to afford ethyl 5-(4-bromo-1-(tetrahydro-2H-pyran-2-yl)-1H-indazol-5-yl)pentanoate (560 mg, 1.37 mmol, 77.4%).

MS (ESI): m/z (+)=409.2.

### Step F: Synthesis of ethyl 5-(1-(tetrahydro-2H-pyran-2-yl)-4-(4,4,5,5-tetramethyl-1,3,2-dioxaborolan-2-yl)-1H-indazol-5-yl)pentanoate

A solution of ethyl 5-(4-bromo-1-(tetrahydro-2H-pyran-2-yl)-1H-indazol-5-yl)pentanoate (560 mg, 1.37 mmol), bis(pinacolato)diboron (695 mg, 2.74 mmol), potassium acetate (269 mg, 2.74 mmol), and Pd(dppf)Cl₂·CH₂Cl₂ (112 mg, 137 μmol) in 1,4-dioxane (20 mL) was degassed with nitrogen and warmed at 100° C. for 16 hours. The reaction mixture was concentrated onto celite and purified by flash column chromatography (ethyl acetate/heptane) to afford ethyl 5-(1-(tetrahydro-2H-pyran-2-yl)-4-(4,4,5,5-tetramethyl-1,3,2-dioxaborolan-2-yl)-1H-indazol-5-yl)pentanoate (728 mg, 0.64 mmol, 47%).

MS (ESI): m/z (+)=457.4.

### Example 10. Synthesis of 3³-chloro-2⁸-fluoro-2²-(((2R,7aS)-2-fluorotetrahydro-1H-pyrrolizin-7a(5H)-yl)methoxy)-3⁵-hydroxy-7-aza-2(4,7)-pyrido[4,3-d]pyrimidina-1(1,3)-piperidina-3(1,2)-benzenacyclononaphan-6-one (Compound 113a)

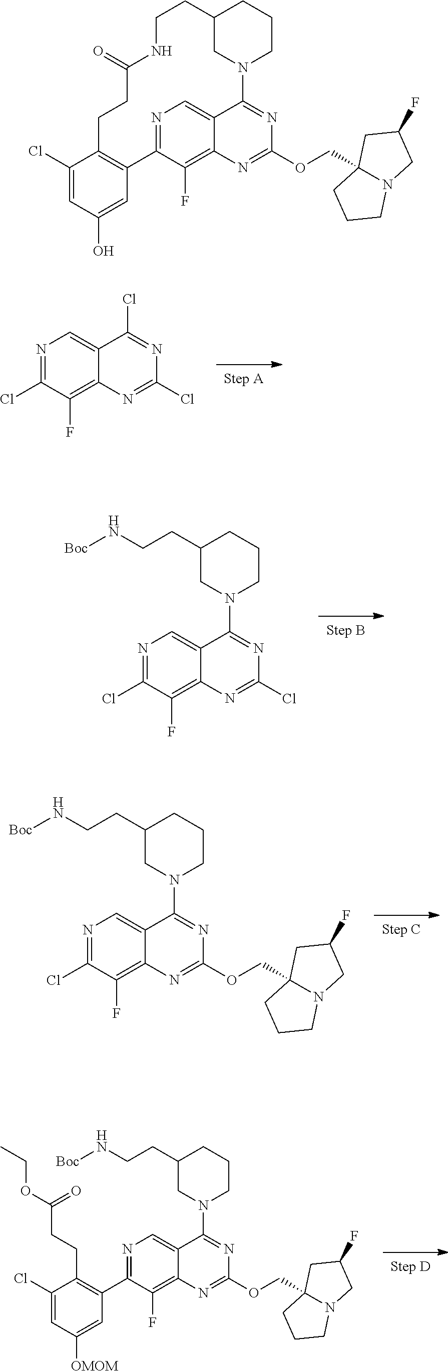

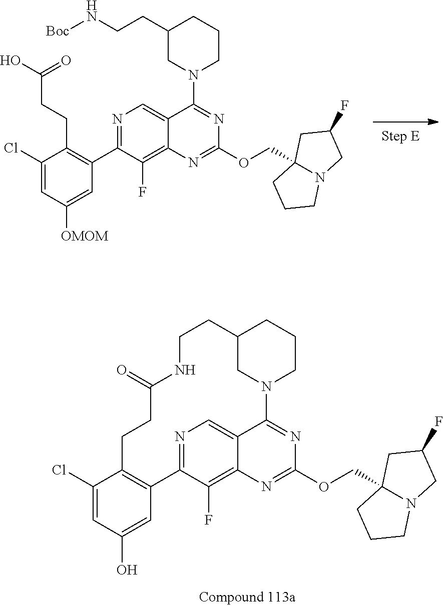

### Step A: Synthesis of tert-butyl (2-(1-(2,7-dichloro-8-fluoropyrido[4,3-d]pyrimidin-4-yl)piperidin-3-yl)ethyl)carbamate

A solution of 2,4,7-trichloro-8-fluoropyrido[4,3-d]pyrimidine (553 mg, 1 equiv., 2.19 mmol) in dichloromethane (10 mL) was cooled to −78° C. A solution of tert-butyl (2-(piperidin-3-yl)ethyl)carbamate (500 mg, 1 equiv., 2.19 mmol) and DIPEA (1.13 g, 1.53 mL, 4 equiv., 8.76 mmol) in dichloromethane (5 mL) was added dropwise. The reaction mixture was stirred at −78° C. for 1 hour. The reaction mixture was diluted with dichloromethane and washed with water and brine, dried (Na₂SO₄), filtered, and concentrated. The resulting residue was purified by flash column chromatography (ethyl acetate/heptane) to afford tert-butyl (2-(1-(2,7-dichloro-8-fluoropyrido[4,3-d]pyrimidin-4-yl)piperidin-3-yl)ethyl)carbamate (437 mg, 984 μmol, 44.9%) as a yellow solid.

MS (ESI): m/z (+)=444.4

### Step B: Synthesis of tert-butyl (2-(1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)ethyl)carbamate

A solution of tert-butyl (2-(1-(2,7-dichloro-8-fluoropyrido[4,3-d]pyrimidin-4-yl)piperidin-3-yl)ethyl)carbamate (1.78 g, 1 equiv., 4.01 mmol), ((2R,7aS)-2-fluorotetrahydro-1H-pyrrolizin-7a(5H)-yl)methanol (765 mg, 1.2 equiv., 4.81 mmol), cataCXium A Pd G3 (292 mg, 0.1 equiv., 401 μmol), and cesium carbonate (3.92 g, 3.0 equiv., 12.0 mmol) in 1,4-dioxane (15 mL) was degassed with nitrogen and warmed at 70° C. for 8 hours. The reaction mixture was filtered through celite, and the filter cake was washed with ethyl acetate. The filtrate was concentrated, and the resulting residue was purified by flash column chromatography using a gradient of 5-100% B (A: heptane; B: ethyl acetate+10% ammonia in methanol) to afford tert-butyl (2-(1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)ethyl)carbamate (2.17 g, 3.83 mmol, 95.5%) as a yellow solid.

MS (ESI): m/z (+)=567.2

### Step C: Synthesis of ethyl 3-(2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-(methoxymethoxy)phenyl)propanoate

A solution of tert-butyl (2-(1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)ethyl)carbamate (140 mg, 1 equiv., 247 μmol), ethyl 3-(2-chloro-4-(methoxymethoxy)-6-(4,4,5,5-tetramethyl-1,3,2-dioxaborolan-2-yl)phenyl)propanoate (108 mg, 1.1 equiv., 272 μmol), cesium carbonate (241 mg, 3.0 equiv., 741 μmol), and cataCXium A Pd G3 (18.0 mg, 0.1 equiv., 24.7 μmol) in 1,4-dioxane (2.0 mL) was degassed with nitrogen and warmed at 100° C. for 4 hours. Ethyl 3-(2-chloro-4-(methoxymethoxy)-6-(4,4,5,5-tetramethyl-1,3,2-dioxaborolan-2-yl)phenyl)propanoate (108 mg, 1.1 equiv., 272 μmol) and cataCXium A Pd G3 (18.0 mg, 0.1 equiv., 24.7 μmol) were added, and the reaction was warmed at 100° C. for 1 hour. The reaction mixture was filtered through celite, and the filter cake was washed with ethyl acetate. The filtrate was concentrated and purified by flash column chromatography using a gradient of 5-90% B (A: heptane; B: ethyl acetate+10% ammonia in methanol) followed by HPLC (Method A) to afford ethyl 3-(2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-(methoxymethoxy)phenyl)propanoate (65 mg, 81 μmol, 33%) as a yellow solid.

MS (ESI): m/z (+)=803.6

### Step D: Synthesis of 3-(2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-(methoxymethoxy)phenyl)propanoic acid

A solution of ethyl 3-(2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-(methoxymethoxy)phenyl)propanoate (65 mg, 1 equiv., 81 μmol) in 1,4-dioxane (1.0 mL) was treated with sodium hydroxide (4.9 mg, 0.12 mL, 1.0 M, 1.5 equiv., 0.12 mmol). The mixture was stirred at room temperature for 1 hour. Sodium hydroxide (4.9 mg, 0.12 mL, 1.0 M, 1.5 equiv., 0.12 mmol) was added, and the reaction mixture was stirred at room temperature for 2 hours. The reaction mixture was brought to pH~7 by the addition of 1M HCl (5 equiv.). The resulting mixture was extracted with ethyl acetate, washed with brine, dried over Na₂SO₄, filtered, and concentrated to afford 3-(2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-(methoxymethoxy)phenyl)propanoic acid (45 mg, 58 μmol, 72%) as a yellow solid.

MS (ESI): m/z (+)=775.5

### Step E: Synthesis of 3³-chloro-2⁸-fluoro-2²-(((2R,7aS)-2-fluorotetrahydro-1H-pyrrolizin-7a(5H)-yl)methoxy)-3⁵-hydroxy-7-aza-2(4,7)-pyrido[4,3-d]pyrimidina-1(1,3)-piperidina-3(1,2)-benzenacyclononaphan-6-one

A solution of 3-(2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-(methoxymethoxy)phenyl)propanoic acid (45 mg, 1 equiv., 58 μmol) in 1,4-dioxane (0.5 mL) was treated with HCl (72.9 mg, 500 μL, 4.0 M, 34 equiv., 2.00 mmol) (4.0 M in 1,4-dioxane). The mixture was stirred at room temperature for 1 hour. The volatiles were removed under reduced pressure. The resulting residue was taken up in DMF (4.0 mL), DIPEA (22 mg, 30 μL, 3.0 equiv., 0.17 mmol), and HATU (24 mg, 1.1 equiv., 63 μmol). The reaction mixture was stirred at room temperature for 30 min. The reaction mixture was purified by HPLC (Method A) to afford 3³-chloro-2⁸-fluoro-2²-(((2R,7aS)-2-fluorotetrahydro-1H-pyrrolizin-7a(5H)-yl)methoxy)-3⁵-hydroxy-7-aza-2(4,7)-pyrido[4,3-d]pyrimidina-1(1,3)-piperidina-3(1,2)-benzenacyclononaphan-6-one (7 mg, 0.01 mmol, 20%) as a white solid.

MS (ESI): m/z (+)=613.4.

¹H NMR (400 MHz, DMSO) δ 10.03 (s, 1H), 9.05 (s, 1H), 7.60-7.53 (m, 1H), 6.98 (d, J=2.5 Hz, 1H), 6.93 (d, J=2.5 Hz, 1H), 5.38-5.17 (m, 1H), 4.56-4.46 (m, 1H), 4.46-4.38 (m, 1H), 4.19-4.09 (m, 1H), 4.09-3.97 (m, 1H), 3.31 (s, 10H), 2.46-1.46 (m, 11H), 1.44-1.13 (m, 4H).

### Example 21. Synthesis of 3³-chloro-2⁸-fluoro-2²-(((2R,7aS)-2-fluorotetrahydro-1H-pyrrolizin-7a(5H)-yl)methoxy)-3⁵-hydroxy-9-aza-2(4,7)-pyrido[4,3-d]pyrimidina-1(1,3)-piperidina-3(1,2)-benzenacycloundecaphan-8-one (Compound 115a)

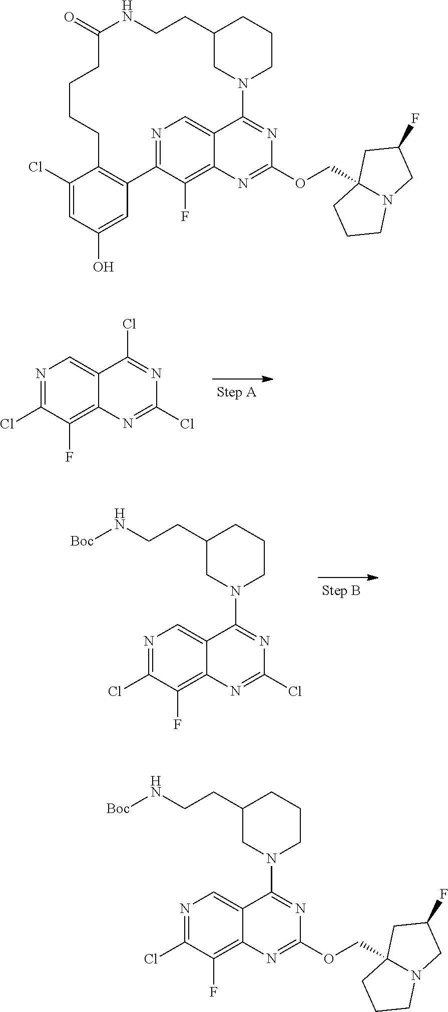 [SMILES: O=C1CCCCc2c(Cl)cc(O)cc2-c2ncc3c(nc(OC[C@@]45CCCN4C[C@H](F)C5)nc3c2F)N2CCCC(CCN1)C2 ; Fc1c(Cl)ncc2c(Cl)nc(Cl)nc12 ; CC(C)(C)OC(=O)NCCC1CCCN(c2nc(Cl)nc3c(F)c(Cl)ncc23)C1 ; CC(C)(C)OC(=O)NCCC1CCCN(c2nc(OC[C@@]34CCCN3C[C@H](F)C4)nc3c(F)c(Cl)ncc23)C1]

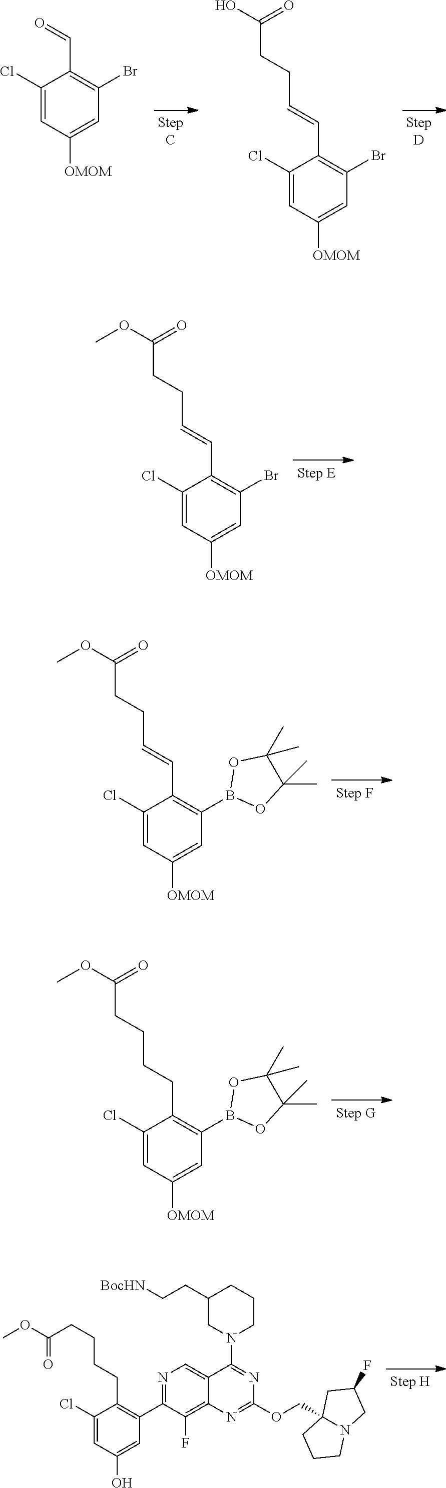

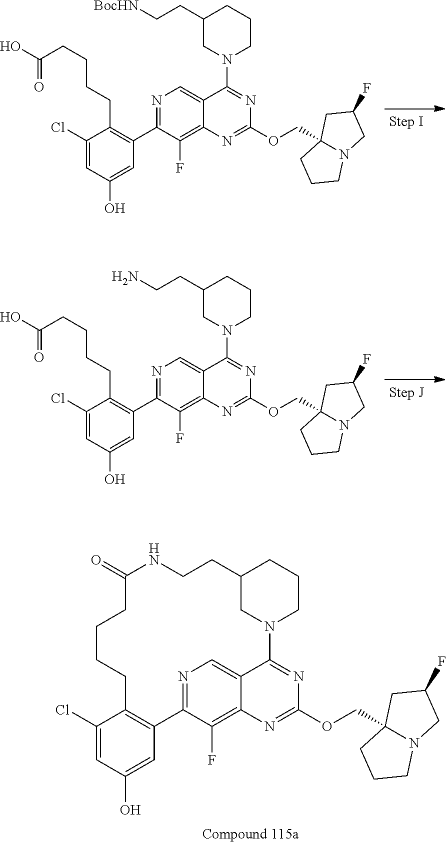 [SMILES: CC(C)(C)OC(=O)NCCC1CCCN(c2nc(OC[C@@]34CCCN3C[C@H](F)C4)nc3c(F)c(-c4cc(O)cc(Cl)c4CCCCC(=O)O)ncc23)C1 ; NCCC1CCCN(c2nc(OC[C@@]34CCCN3C[C@H](F)C4)nc3c(F)c(-c4cc(O)cc(Cl)c4CCCCC(=O)O)ncc23)C1 ; O=C1CCCCc2c(Cl)cc(O)cc2-c2ncc3c(nc(OC[C@@]45CCCN4C[C@H](F)C5)nc3c2F)N2CCCC(CCN1)C2]

### Step A: Synthesis of tert-butyl (2-(1-(2,7-dichloro-8-fluoropyrido[4,3-d]pyrimidin-4-yl)piperidin-3-yl)ethyl)carbamate

A solution of 2,4,7-trichloro-8-fluoropyrido[4,3-d]pyrimidine (1.00 g, 1 equiv., 3.96 mmol) in dichloromethane (15 mL) was cooled to −78° C. A solution of tert-butyl (2-(piperidin-3-yl)ethyl)carbamate (904 mg, 1 equiv., 3.96 mmol) and DIPEA (2.05 g, 2.76 mL, 4 equiv., 15.8 mmol) in dichloromethane (5 mL) was added dropwise. The reaction mixture was stirred at −78° C. After 1 hour, the reaction was partitioned between dichloromethane and water. The organic layer was then washed with brine, dried over sodium sulfate, filtered, and concentrated. Purification via flash column chromatography using a gradient of 0-75% B (A: heptane; B: EtOAc) gave tert-butyl (2-(1-(2,7-dichloro-8-fluoropyrido[4,3-d]pyrimidin-4-yl)piperidin-3-yl)ethyl)carbamate (1.51 g, 86% yield).

MS (ESI): m/z=444.2 [M+H]⁺.

### Step B: Synthesis of tert-butyl (2-(1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)ethyl)carbamate

A solution of tert-butyl (2-(1-(2,7-dichloro-8-fluoropyrido[4,3-d]pyrimidin-4-yl)piperidin-3-yl)ethyl)carbamate (1.78 g, 1 equiv., 4.01 mmol), ((2R,7aS)-2-fluorotetrahydro-1H-pyrrolizin-7a(5H)-yl)methanol (765 mg, 1.2 equiv., 4.81 mmol), cataCXium A Pd G3 (292 mg, 0.1 equiv., 401 μmol), and cesium carbonate (3.92 g, 3.0 equiv., 12.0 mmol) in 1,4-dioxane (15 mL) was degassed with nitrogen. The reaction mixture was heated to 70° C. After 8 hours, the reaction mixture was filtered through celite and concentrated. Purification via flash column chromatography using a gradient of 5-100% (A: heptane; B: EtOAc+10% 7N ammonia in methanol) gave tert-butyl (2-(1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)ethyl)carbamate (2.17 g, 96% yield).

MS (ESI): m/z=567.4 [M+H]⁺.

### Step C: Synthesis of (E)-5-(2-bromo-6-chloro-4-(methoxymethoxy)phenyl)pent-4-enoic acid

To a solution of (3-carboxypropyl)triphenylphosphonium bromide (5.8 g, 1.25 equiv., 13 mmol) and 2-bromo-6-chloro-4-(methoxymethoxy)benzaldehyde (3.0 g, 1 equiv., 11 mmol) in tetrahydrofuran (40 mL) at 0° C. was added potassium tert-butoxide (3.0 g, 27 mL, 1.0 M, 2.5 equiv., 27 mmol) dropwise over 10 minutes. The mixture was allowed to warm to room temperature. After 16 hours, the reaction mixture was diluted with water and extracted into ethyl acetate. The aqueous layer was adjusted to pH=3 by the addition of HCl (1 M) and extracted with ethyl acetate once more. The combined organic layers were further washed with brine, dried over sodium sulfate, filtered, and concentrated. Purification via flash column chromatography using a gradient of 0-100% B (A: heptane; B: EtOAc) gave (E)-5-(2-bromo-6-chloro-4-(methoxymethoxy)phenyl)pent-4-enoic acid (2.42 g, 64% yield).

MS (ESI): m/z=351.0 [M+H]⁺.

### Step D: Synthesis of methyl (E)-5-(2-bromo-6-chloro-4-(methoxymethoxy)phenyl)pent-4-enoate

To a solution of (E)-5-(2-bromo-6-chloro-4-(methoxymethoxy)phenyl)pent-4-enoic acid (3.21 g, 1 equiv., 9.18 mmol) and cesium carbonate (7.48 g, 2.5 equiv., 23.0 mmol) in acetone (30 mL) was added iodomethane (1.95 g, 861 μL, 1.5 equiv., 13.8 mmol). The reaction mixture was heated to 60° C. After 1 hour, the reaction mixture was filtered through celite and concentrated. Purification via flash column chromatography using a gradient of 0-80% B (A: heptane; B: EtOAc) gave methyl (E)-5-(2-bromo-6-chloro-4-(methoxymethoxy)phenyl)pent-4-enoate (3.22 g, 96% yield).

MS (ESI): m/z=363.1 [M+H]⁺.

### Step E: Synthesis of methyl (E)-5-(2-chloro-4-(methoxymethoxy)-6-(4,4,5,5-tetramethyl-1,3,2-dioxaborolan-2-yl)phenyl)pent-4-enoate

To a solution of methyl (E)-5-(2-bromo-6-chloro-4-(methoxymethoxy)phenyl)pent-4-enoate (3.11 g, 1 equiv., 8.55 mmol) in 1,4-dioxane (50 mL) was added potassium acetate (2.52 g, 3 equiv., 25.7 mmol), bis(pinacolato)diboron (4.34 g, 2 equiv., 17.1 mmol), and Pd(dppf)Cl₂·CH₂Cl₂ (698 mg, 0.1 equiv., 855 μmol). The reaction mixture was heated to 100° C. After 1 hour, the reaction mixture was filtered through celite and concentrated. Purification via flash column chromatography using a gradient of 0-25% B (A: heptane; B: EtOAc) gave methyl (E)-5-(2-chloro-4-(methoxymethoxy)-6-(4,4,5,5-tetramethyl-1,3,2-dioxaborolan-2-yl)phenyl)pent-4-enoate (2.84 g, 81% yield).

MS (ESI): m/z=411.5 [M+H]⁺.

### Step F: Synthesis of methyl 5-(2-chloro-4-(methoxymethoxy)-6-(4,4,5,5-tetramethyl-1,3,2-dioxaborolan-2-yl)phenyl)pentanoate

To a solution of methyl (E)-5-(2-chloro-4-(methoxymethoxy)-6-(4,4,5,5-tetramethyl-1,3,2-dioxaborolan-2-yl)phenyl)pent-4-enoate (1050 mg, 1 equiv., 2.557 mmol) in methanol (15 mL) was added Pd/C (105 mg, 0.386 equiv., 987 μmol). The flask was evacuated (3×) and placed under hydrogen (balloon). The reaction mixture was stirred at room temperature. After 90 minutes, the reaction mixture was filtered through celite, and the filter pad was rinsed with methanol. The filtrate was concentrated to give methyl 5-(2-chloro-4-(methoxymethoxy)-6-(4,4,5,5-tetramethyl-1,3,2-dioxaborolan-2-yl)phenyl)pentanoate (717 mg, 68%), which was immediately used in the following step.

MS (ESI): m/z=435.3 [M+Na]⁺.

### Step G: Synthesis of methyl 5-(2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-hydroxyphenyl)pentanoate

To a solution of tert-butyl (2-(1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)ethyl)carbamate (590 mg, 1 equiv., 1.04 mmol) in 1,4-dioxane (10 mL) was added methyl 5-(2-chloro-4-(methoxymethoxy)-6-(4,4,5,5-tetramethyl-1,3,2-dioxaborolan-2-yl)phenyl)pentanoate (644 mg, 1.5 equiv., 1.56 mmol), cesium carbonate (1.02 g, 3.0 equiv., 3.12 mmol), and cataCXium A Pd G3 (75.8 mg, 0.1 equiv., 104 μmol). The reaction mixture was degassed with nitrogen and heated to 100° C. After 2 hours, the reaction mixture was filtered through celite and concentrated. The resulting residue was purified by HPLC (Method A) to afford methyl 5-(2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-hydroxyphenyl)pentanoate (320 mg, 40% yield).

MS (ESI): m/z=773.8 [M+H]⁺.

### Step H: Synthesis of 5-(2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-hydroxyphenyl)pentanoic acid

To a solution of methyl 5-(2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-hydroxyphenyl)pentanoate (55 mg, 1 equiv., 71 μmol) in 1,4-dioxane (1.0 mL) was added sodium hydroxide (8.5 mg, 0.21 mL, 1.0 M, 3 equiv., 0.21 mmol). The reaction mixture was stirred at room temperature. After 6 hours, the reaction mixture was adjusted to pH~7 by addition of 1.0 M HCl (3 equiv.) and partitioned between ethyl acetate and water. The organic layer was further washed with brine, dried over sodium sulfate, filtered, and evaporated to dryness. The crude 5-(2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-hydroxyphenyl)pentanoic acid (168 mg, 52% yield) was used as is in the next step without further purification.

MS (ESI): m/z=760.3 [M+H]⁺.

### Step I: Synthesis of 5-(2-(4-(3-(2-aminoethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-hydroxyphenyl)pentanoic acid

To a solution of 5-(2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-hydroxyphenyl)pentanoic acid (195 mg, 1 equiv., 257 μmol) in 1,4-dioxane (2.0 mL) was added HCl in 1,4-dioxane (0.15 g, 1.0 mL, 4.0 M, 16 equiv., 4.0 mmol). The reaction mixture was stirred at room temperature. After 1 hour, the reaction mixture was evaporated to dryness to give 5-(2-(4-(3-(2-aminoethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-hydroxyphenyl)pentanoic acid as a crude residue (212 mg, 114% yield), which was used in the next step without further purification MS (ESI): m/z=659.9 [M+H]⁺.

### Step J: Synthesis of 3³-chloro-2⁸-fluoro-2²-(((2R,7aS)-2-fluorotetrahydro-1H-pyrrolizin-7a(5H)-yl)methoxy)-3⁵-hydroxy-9-aza-2(4,7)-pyrido[4,3-d]pyrimidina-1(1,3)-piperidina-3(1,2)-benzenacycloundecaphan-8-one

To a solution of 5-(2-(4-(3-(2-aminoethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chloro-4-hydroxyphenyl)pentanoic acid, HCl (173 mg, 1 equiv., 249 μmol) in DMF (13 mL) was added DIPEA (129 mg, 173 μL, 4.0 equiv., 995 μmol) and HATU (104 mg, 1.1 equiv., 274 μmol). The reaction mixture was stirred at room temperature. After 36 hours, the reaction mixture was filtered and concentrated. The resulting residue was purified by HPLC (Method A) to afford 3³-chloro-2⁸-fluoro-2²-(((2R,7aS)-2-fluorotetrahydro-1H-pyrrolizin-7a(5H)-yl)methoxy)-3⁵-hydroxy-9-aza-2(4,7)-pyrido[4,3-d]pyrimidina-1(1,3)-piperidina-3(1,2)-benzenacycloundecaphan-8-one (61 mg, 38% yield).

MS (ESI): m/z=641.8 [M+H]⁺.

¹H NMR (400 MHz, DMSO) δ 9.92 (s, 1H), 9.06 (s, 1H), 7.40-7.35 (m, 1H), 6.98-6.93 (m, 1H), 6.83-6.78 (m, 1H), 5.37-5.18 (m, 1H), 4.66-4.40 (m, 2H), 4.18-4.08 (m, 1H), 4.08-4.00 (m, 1H), 3.40-3.33 (m, 1H), 3.24-2.96 (m, 6H), 2.92-2.77 (m, 2H), 2.64-2.53 (m, 1H), 2.35-2.24 (m, 1H), 2.15-1.94 (m, 4H), 1.88-1.64 (m, 7H), 1.50-1.20 (m, 4H), 1.20-1.07 (m, 3H).

### Example 22. Synthesis of (1³R)-3³-chloro-2⁸-fluoro-2²-(((2R,7aS)-2-fluorotetrahydro-1H-pyrrolizin-7a(5H)-yl)methoxy)-3⁵-hydroxy-9-aza-2(4,7)-pyrido[4,3-d]pyrimidina-1(1,3)-piperidina-3(1,2)-benzenacycloundecaphan-8-one (Compound 115b)

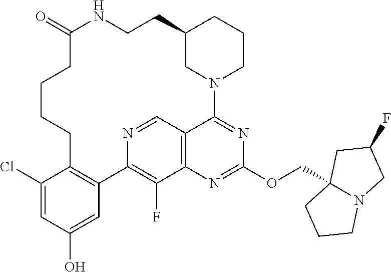 [SMILES: O=C1CCCCc2c(Cl)cc(O)cc2-c2ncc3c(nc(OC[C@@]45CCCN4C[C@H](F)C5)nc3c2F)N2CCC[C@H](CCN1)C2]

The diastereomers of Compound 115a (prepared according to Example 21) were separated using Prep-HPLC with the following conditions: Column: Chiralpak IC 20×250 mm, 5 μM; Mobile Phase A: water (0.1% formic acid), Mobile Phase B: acetonitrile (0.1% formic acid); Flow rate: 18.9 mL/min; Gradient: 10% B to 42% B in 16.9 min; Wavelength: 214 nm; Retention time of the first eluting peak was 8.9 min (Compound 115b; MS (ESI): m/z=641.4 [M+H]⁺.

### Example 23. Synthesis of (1³S)-3³-chloro-2⁸-fluoro-2²-(((2R,7aS)-2-fluorotetrahydro-1H-pyrrolizin-7a(5H)-yl)methoxy)-3⁵-hydroxy-9-aza-2(4,7)-pyrido[4,3-d]pyrimidina-1(1,3)-piperidina-3(1,2)-benzenacycloundecaphan-8-one (Compound 115c)

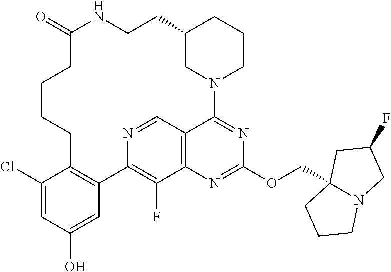 [SMILES: O=C1CCCCc2c(Cl)cc(O)cc2-c2ncc3c(nc(OC[C@@]45CCCN4C[C@H](F)C5)nc3c2F)N2CCC[C@@H](CCN1)C2]

The diastereomers of Compound 115a (prepared according to Example 21) were separated using Prep-HPLC with the following conditions: Column: Chiralpak IC 20×250 mm, 5 μM; Mobile Phase A: water (0.1% formic acid), Mobile Phase B: acetonitrile (0.1% formic acid); Flow rate: 18.9 mL/min; Gradient: 10% B to 42% B in 16.9 min; Wavelength: 214 nm; Retention time of the second eluting peak was 10.6 min (Compound 115c; MS (ESI): m/z=641.4.

### Example 24. (Z)-1³-chloro-2⁸-fluoro-2²-(((2R,7aS)-2-fluorotetrahydro-1H-pyrrolizin-7a(5H)-yl)methoxy)-5¹H-2(7,4)-pyrido[4,3-d]pyrimidina-3(1,3)-piperidina-5(4,1)-triazola-1(1,2)-benzenacyclooctaphan-1⁵-ol (Compound 121a)

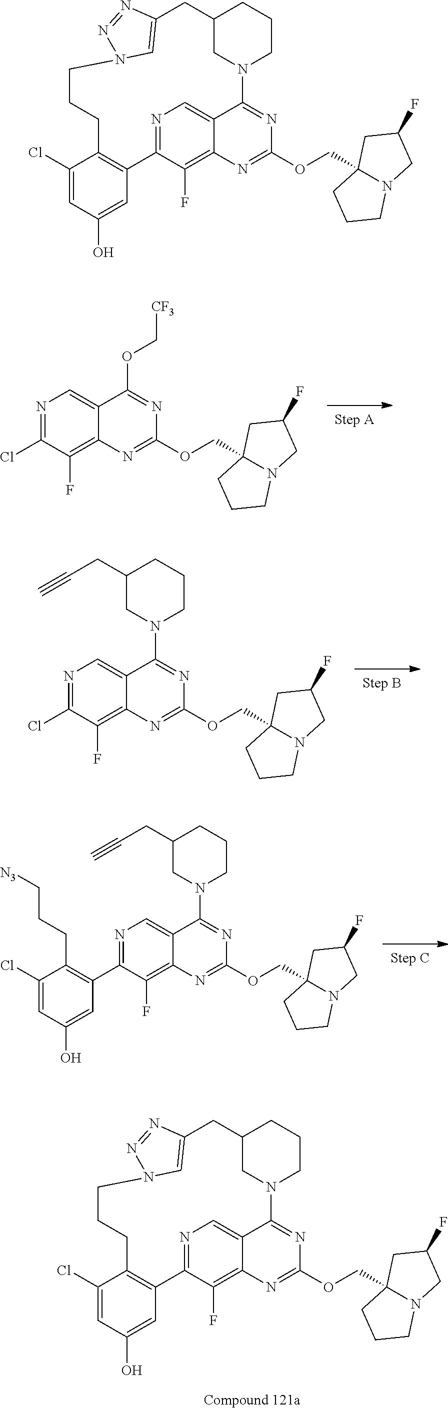 [SMILES: Oc1cc(Cl)c2c(c1)-c1ncc3c(nc(OC[C@@]45CCCN4C[C@H](F)C5)nc3c1F)N1CCCC(Cc3cn(nn3)CCC2)C1 ; Fc1c(Cl)ncc2c(OCC(F)(F)F)nc(OC[C@@]34CCCN3C[C@H](F)C4)nc12 ; C#CCC1CCCN(c2nc(OC[C@@]34CCCN3C[C@H](F)C4)nc3c(F)c(Cl)ncc23)C1 ; C#CCC1CCCN(c2nc(OC[C@@]34CCCN3C[C@H](F)C4)nc3c(F)c(-c4cc(O)cc(Cl)c4CCCN=[N+]=[N-])ncc23)C1 ; Oc1cc(Cl)c2c(c1)-c1ncc3c(nc(OC[C@@]45CCCN4C[C@H](F)C5)nc3c1F)N1CCCC(Cc3cn(nn3)CCC2)C1]

### Step A: Synthesis of 7-chloro-8-fluoro-2-(((2R,7aS)-2-fluorotetrahydro-1H-pyrrolizin-7a(5H)-yl)methoxy)-4-(3-(prop-2-yn-1-yl)piperidin-1-yl)pyrido[4,3-d]pyrimidine

A solution of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (300 mg, 1 equiv., 684 μmol), 3-(prop-2-yn-1-yl)piperidine, HCl (109 mg, 1 equiv., 684 μmol) and DIPEA (884 mg, 1.19 mL, 10 equiv., 6.84 mmol) in DMSO (3 mL) under nitrogen was warmed at 50° C. for 3 days. The reaction mixture was partially concentrated and purified by HPLC (Method A) to afford 7-chloro-8-fluoro-2-(((2R,7aS)-2-fluorotetrahydro-1H-pyrrolizin-7a(5H)-yl)methoxy)-4-(3-(prop-2-yn-1-yl)piperidin-1-yl)pyrido[4,3-d]pyrimidine (214 mg, 463 μmol, 67.8%) as an off-white solid.

MS (ESI): m/z (+)=462.3.

### Step B: Synthesis of 4-(3-azidopropyl)-3-chloro-5-(8-fluoro-2-(((2R,7aS)-2-fluorotetrahydro-1H-pyrrolizin-7a(5H)-yl)methoxy)-4-(3-(prop-2-yn-1-yl)piperidin-1-yl)pyrido[4,3-d]pyrimidin-7-yl)phenol

A solution of 7-chloro-8-fluoro-2-(((2R,7aS)-2-fluorotetrahydro-1H-pyrrolizin-7a(5H)-yl)methoxy)-4-(3-(prop-2-yn-1-yl)piperidin-1-yl)pyrido[4,3-d]pyrimidine (45 mg, 1 equiv., 98 μmol), 4-(3-azidopropyl)-3-chloro-5-(4,4,5,5-tetramethyl-1,3,2-dioxaborolan-2-yl)phenol (33 mg, 1 equiv., 98 μmol), cataCXium A Pd G3 (7.1 mg, 0.1 equiv., 9.8 μmol), and potassium phosphate (62 mg, 0.20 mL, 1.5 M in water, 3 equiv., 0.29 mmol) in 1,4-dioxane (3 mL) was degassed with nitrogen and warmed at 80° C. for 30 min in a microwave reactor. The reaction mixture was filtered, and the filtrate was concentrated and purified by HPLC (Method A) to afford 4-(3-azidopropyl)-3-chloro-5-(8-fluoro-2-(((2R,7aS)-2-fluorotetrahydro-1H-pyrrolizin-7a(5H)-yl)methoxy)-4-(3-(prop-2-yn-1-yl)piperidin-1-yl)pyrido[4,3-d]pyrimidin-7-yl)phenol (21 mg, 33 μmol, 34%) as an off-white solid.

MS (ESI): m/z (+)=637.3.

### Step C: Synthesis of (Z)-13-chloro-28-fluoro-22-(((2R,7aS)-2-fluorotetrahydro-1H-pyrrolizin-7a(5H)-yl)methoxy)-51H-2(7,4)-pyrido[4,3-d]pyrimidina-3(1,3)-piperidina-5(4,1)-triazola-1(1,2)-benzenacyclooctaphan-15-ol

A suspension of 4-(3-azidopropyl)-3-chloro-5-(8-fluoro-2-(((2R,7aS)-2-fluorotetrahydro-1H-pyrrolizin-7a(5H)-yl)methoxy)-4-(3-(prop-2-yn-1-yl)piperidin-1-yl)pyrido[4,3-d]pyrimidin-7-yl)phenol (21 mg, 1 equiv., 33 μmol), copper (II) sulfate (1.1 mg, 0.2 equiv., 6.6 μmol), and sodium 2-(1,2-dihydroxy-ethyl)-4-hydroxy-5-oxo-2,5-dihydro-furan-3-olate (1.3 mg, 0.2 equiv., 6.6 μmol) in tert-Amyl alcohol (1 mL), THF (2 mL), and H₂O (0.5 mL) was degassed with nitrogen. The mixture was warmed at 50° C. for 20 min. The reaction mixture was concentrated and purified by HPLC (Method A) to afford (Z)-13-chloro-28-fluoro-22-(((2R,7aS)-2-fluorotetrahydro-1H-pyrrolizin-7a(5H)-yl)methoxy)-51H-2(7,4)-pyrido[4,3-d]pyrimidina-3(1,3)-piperidina-5(4,1)-triazola-1(1,2)-benzenacyclooctaphan-15-ol (11 mg, 17 μmol, 52%) as a white solid.

MS (ESI): m/z (+)=637.2.

¹H NMR (400 MHz, DMSO) δ 10.02 (s, 1H), 8.64 (s, 1H), 7.74 (s, 1H), 6.98 (d, J=2.5 Hz, 1H), 6.88-6.81 (m, 1H), 5.38-5.13 (m, 1H), 4.41-4.29 (m, 2H), 4.22-4.09 (m, 2H), 4.07-4.01 (m, 1H), 4.01-3.93 (m, 1H), 3.62-3.43 (m, 2H), 3.13-2.98 (m, 3H), 2.87-2.77 (m, 1H), 2.67-2.57 (m, 1H), 2.49-2.30 (m, 2H), 2.23-2.10 (m, 2H), 2.10-1.50 (m, 12H).

### Example 25. (Z)-2⁸,3⁷-difluoro-2²-(((2R,7aS)-2-fluorotetrahydro-1H-pyrrolizin-7a(5H)-yl)methoxy)-4¹H-2(4,7)-pyrido[4,3-d]pyrimidina-1(1,3)-piperidina-4(4,1)-triazola-3(1,8)-naphthalenacyclooctaphan-3³-ol (Compound 125a)

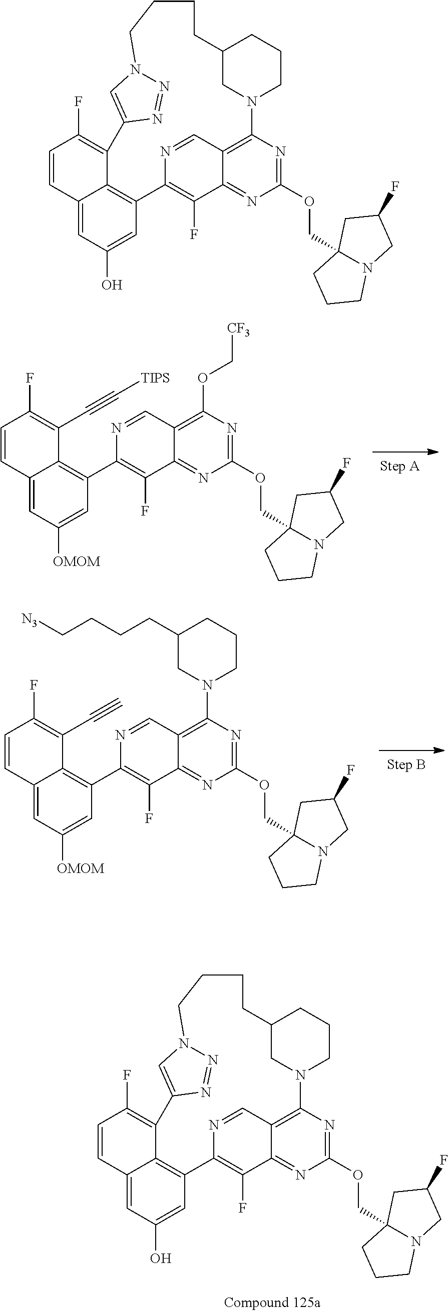

### Step A: Synthesis of 4-(3-(4-azidobutyl)piperidin-1-yl)-7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidine

A solution of 8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine (100 mg, 1.0 equiv., 114 μmol), 3-(4-azidobutyl)piperidine, HCl (37.4 mg, 1.5 equiv., 171 μmol), and CsF (86.6 mg, 5 equiv., 570 μmol) in DMSO (1.0 mL) was warmed at 50° C. for 18 hours. The reaction mixture was purified by HPLC (Method A) to afford 4-(3-(4-azidobutyl)piperidin-1-yl)-7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidine (65 mg, 91 μmol, 80%).

MS (ESI): m/z (+)=715.5.

### Step B: Synthesis of (Z)-2⁸,3⁷-difluoro-2²-(((2R,7aS)-2-fluorotetrahydro-1H-pyrrolizin-7a(5H)-yl)methoxy)-4¹H-2(4,7)-pyrido[4,3-d]pyrimidina-1(1,3)-piperidina-4(4,1)-triazola-3(1,8)-naphthalenacyclooctaphan-3³-ol

A solution of 4-(3-(4-azidobutyl)piperidin-1-yl)-7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidine (65 mg, 1 equiv., 91 μmol) in tert-Amyl alcohol (1.0 mL) and THF (2.0 mL) at room temperature was treated with a mixture of sodium 2-(1,2-dihydroxy-ethyl)-4-hydroxy-5-oxo-2,5-dihydro-furan-3-olate (3.6 mg, 0.2 equiv., 18 μmol) and copper (II) sulfate (2.9 mg, 1.3 μL, 0.2 equiv., 18 μmol) in water (0.5 mL). The reaction mixture was warmed at 50° C. for 1 hour and then cooled to room temperature. The mixture was stirred for 3 days. The reaction mixture was treated with HCl in 1,4-dioxane (66 mg, 0.45 mL, 4.0 M, 20 equiv., 1.8 mmol). The mixture was stirred at room temperature for 1 hour. Additional HCl in 1,4-dioxane (66 mg, 0.45 mL, 4.0 M, 20 equiv., 1.8 mmol) was added, and the reaction mixture was stirred at room temperature for 18 hours. The reaction mixture was concentrated and was purified by HPLC (Method A) to afford (Z)-2⁸,3⁷-difluoro-2²-(((2R,7aS)-2-fluorotetrahydro-1H-pyrrolizin-7a(5H)-yl)methoxy)-4¹H-2(4,7)-pyrido[4,3-d]pyrimidina-1(1,3)-piperidina-4(4,1)-triazola-3(1,8)-naphthalenacyclooctaphan-3³-ol (14.1 mg, 21.0 μmol, 230%).

MS (ESI): m/z (+)=671.5.

¹H NMR (400 MHz, DMSO) δ 10.00 (s, 1H), 8.85 (s, 1H), 8.11 (s, 1H), 8.01-7.95 (m, 1H), 7.51-7.44 (m, 1H), 7.40-7.34 (m, 1H), 7.19-7.13 (m, 1H), 5.35-5.17 (m, 1H), 4.56-4.42 (m, 1H), 4.33-4.22 (m, 2H), 4.17-3.99 (m, 3H), 3.85-3.76 (m, 1H), 3.17-2.95 (m, 4H), 2.91-2.74 (m, 1H), 2.15-0.65 (m, 17H).

### Example 26. 2⁸,3⁷-difluoro-2²-(((2R,7aS)-2-fluorotetrahydro-1H-pyrrolizin-7a(5H)-yl)methoxy)-3³-hydroxy-8-aza-2(4,7)-pyrido[4,3-d]pyrimidina-1(1,3)-piperidina-3(1,8)-naphthalenacyclononaphan-7-one (Compound 135a)

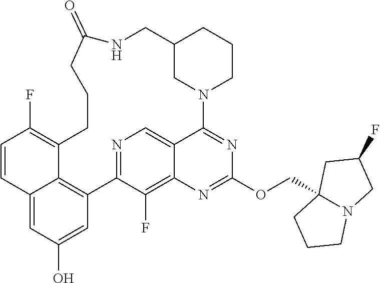 [SMILES: O=C1CCCc2c(F)ccc3cc(O)cc(c23)-c2ncc3c(nc(OC[C@@]45CCCN4C[C@H](F)C5)nc3c2F)N2CCCC(CN1)C2]

2⁸,3⁷-difluoro-2²-(((2R,7aS)-2-fluorotetrahydro-1H-pyrrolizin-7a(5H)-yl)methoxy)-3³-hydroxy-8-aza-2(4,7)-pyrido[4,3-d]pyrimidina-1(1,3)-piperidina-3(1,8)-naphthalenacyclononaphan-7-one was prepared in an analogous manner to Example 10 by using tert-butyl (piperidin-3-ylmethyl)carbamate in Step A and ethyl 4-(2-fluoro-6-(methoxymethoxy)-8-(4,4,5,5-tetramethyl-1,3,2-dioxaborolan-2-yl)naphthalen-1-yl)butanoate in Step C.

MS (ESI): m/z (+)=647.5.

¹H NMR (400 MHz, MeOD) δ 8.93 (s, 1H), 7.74-7.66 (m, 1H), 7.33-7.22 (m, 2H), 7.14-7.09 (m, 1H), 5.47-5.22 (m, 1H), 4.99-4.90 (m, 2H), 4.54-4.27 (m, 4H), 3.99-3.91 (m, 1H), 3.68-3.57 (m, 1H), 3.48-3.30 (m, 2H), 3.10-3.06 (m, 1H), 2.68-2.64 (m, 2H), 2.50-1.33 (m, 16H).

### Example 27. 3³-chloro-2⁸-fluoro-2²-(((2R,7aS)-2-fluorotetrahydro-1H-pyrrolizin-7a(5H)-yl)methoxy)-1³,3⁵-dihydroxy-9-aza-2(4,7)-pyrido[4,3-d]pyrimidina-1(1,3)-piperidina-3(1,2)-benzenacycloundecaphan-8-one (Compound 140a)

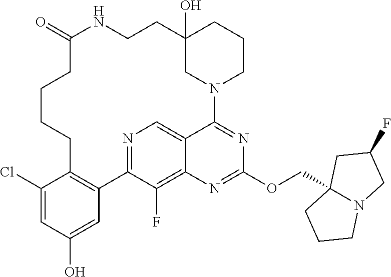 [SMILES: O=C1CCCCc2c(Cl)cc(O)cc2-c2ncc3c(nc(OC[C@@]45CCCN4C[C@H](F)C5)nc3c2F)N2CCCC(O)(CCN1)C2]

3³-chloro-2⁸-fluoro-2²-(((2R,7aS)-2-fluorotetrahydro-1H-pyrrolizin-7a(5H)-yl)methoxy)-1³,3⁵-dihydroxy-9-aza-2(4,7)-pyrido[4,3-d]pyrimidina-1(1,3)-piperidina-3(1,2)-benzenacycloundecaphan-8-one was prepared in an analogous manner to Example 10 by using tert-butyl (2-(3-hydroxypiperidin-3-yl)ethyl)carbamate in Step A and methyl 5-(2-chloro-4-(methoxymethoxy)-6-(4,4,5,5-tetramethyl-1,3,2-dioxaborolan-2-yl)phenyl)pentanoate in Step C.

MS (ESI): m/z (+)=657.5.

### Example 28. 3³-chloro-2⁸-fluoro-2²-(((2R,7aS)-2-fluorotetrahydro-1H-pyrrolizin-7a(5H)-yl)methoxy)-3⁵-hydroxy-9-aza-2(4,7)-pyrido[4,3-d]pyrimidina-1(1,3)-piperidina-3(1,2)-benzenacyclodecaphan-8-one (Compound 141a)

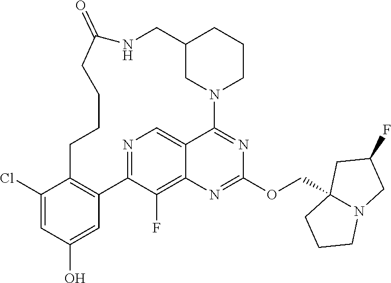 [SMILES: O=C1CCCCc2c(Cl)cc(O)cc2-c2ncc3c(nc(OC[C@@]45CCCN4C[C@H](F)C5)nc3c2F)N2CCCC(CN1)C2]

3³-chloro-2⁸-fluoro-2²-(((2R,7aS)-2-fluorotetrahydro-1H-pyrrolizin-7a(5H)-yl)methoxy)-3⁵-hydroxy-9-aza-2(4,7)-pyrido[4,3-d]pyrimidina-1(1,3)-piperidina-3(1,2)-benzenacyclodecaphan-8-one was prepared in an analogous manner to Example 10 by using tert-butyl (piperidin-3-ylmethyl)carbamate in Step A and methyl 5-(2-chloro-4-(methoxymethoxy)-6-(4,4,5,5-tetramethyl-1,3,2-dioxaborolan-2-yl)phenyl)pentanoate in Step C.

MS (ESI): m/z (+)=627.5.

¹H NMR (400 MHz, MeOD) δ 8.95 (s, 1H), 6.95 (d, J=2.6 Hz, 1H), 6.87-6.82 (m, 1H), 5.40-5.17 (m, 1H), 4.39-4.19 (m, 4H), 3.76-3.56 (m, 2H), 3.28-2.79 (m, 5H), 2.59-2.07 (m, 5H), 2.06-1.70 (m, 9H), 1.58-1.12 (m, 6H).

### Example 29. 3⁵-amino-3³-chloro-2⁸-fluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacycloundecaphan-8-one (Compound 154a)

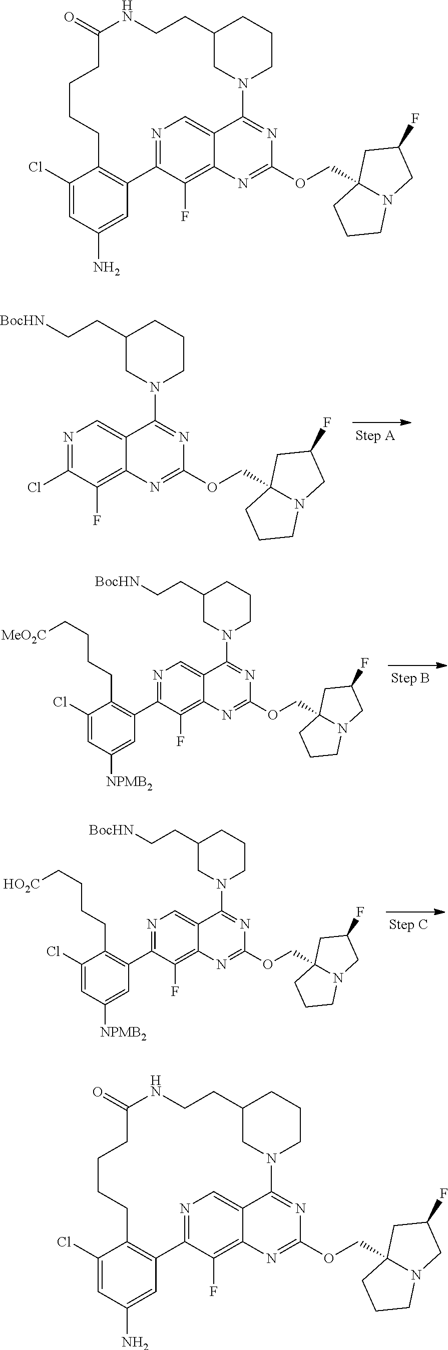

### Step A: Synthesis of methyl 5-(4-(bis(4-methoxybenzyl)amino)-2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chlorophenyl)pentanoate

A solution of tert-butyl (2-(1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)ethyl)carbamate (100 mg, 1 equiv., 176 μmol), methyl 5-(4-(bis(4-methoxybenzyl)amino)-2-chloro-6-(4,4,5,5-tetramethyl-1,3,2-dioxaborolan-2-yl)phenyl)pentanoate (107 mg, 1.0 equiv., 176 μmol), Cs₂CO₃ (172 mg, 3 equiv., 529 μmol), and cataCXium A Pd G3 (12.8 mg, 0.1 equiv., 17.6 μmol) in 1,4-dioxane (4 mL) and water (0.5 mL) was warmed at 100° C. for 1 hour. The reaction mixture was concentrated and was purified by reverse-phase flash column chromatography using a gradient of 10-100% B (A: water+0.1% formic acid; B: acetonitrile+0.1% formic acid) to afford methyl 5-(4-(bis(4-methoxybenzyl)amino)-2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chlorophenyl)pentanoate (69 mg, 68 μmol, 39%).

MS (ESI): m/z (+)=1012.7.

### Step B: Synthesis of 5-(4-(bis(4-methoxybenzyl)amino)-2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chlorophenyl)pentanoic acid

A solution of methyl 5-(4-(bis(4-methoxybenzyl)amino)-2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chlorophenyl)pentanoate (69 mg, 1 equiv., 68 μmol) in THF (4 mL) and water (1 mL) was treated with LiOH·H₂O (14 mg, 5 equiv., 0.34 mmol). The reaction mixture was stirred for 1 hour. LiOH·H₂O (14 mg, 5 equiv., 0.34 mmol) was added and the reaction mixture was stirred at room temperature for 4 hours. The reaction mixture was concentrated and was purified by reverse-phase flash column chromatography using a gradient of 10-100% B (A: water+0.1% formic acid; B: acetonitrile+0.1% formic acid) to afford 5-(4-(bis(4-methoxybenzyl)amino)-2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chlorophenyl)pentanoic acid (54 mg, 54 μmol, 79%).

MS (ESI): m/z (+)=998.7.

### Step C: Synthesis of 3⁵-amino-3³-chloro-2⁸-fluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacycloundecaphan-8-one

A solution of 5-(4-(bis(4-methoxybenzyl)amino)-2-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chlorophenyl)pentanoic acid (54 mg, 1 equiv., 54 μmol) in DCM (1 M) was treated with HCl in 1,4-dioxane (0.20 g, 1.4 mL, 4 M, 100 equiv., 5.4 mmol). The mixture was stirred at room temperature for 60 minutes. The volatiles were removed under a stream of nitrogen, and the residue was further dried under vacuum. The resulting residue was taken up in DMF (4 mL) and treated with HATU (25 mg, 1.2 equiv., 65 μmol) and DIPEA (35 mg, 47 μL, 5 equiv., 0.27 mmol). The mixture was stirred at room temperature for 30 minutes. The reaction mixture was diluted with EtOAc, washed with water and brine, dried (Na₂SO₄), filtered, and concentrated. The resulting residue was treated with TFA (0.62 g, 0.42 mL, 100 equiv., 5.4 mmol). The mixture was stirred at 50° C. for 30 minutes. The volatiles were removed under a stream of nitrogen and the resulting residue was purified by HPLC (Method A) to afford 3⁵-amino-3³-chloro-2⁸-fluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacycloundecaphan-8-one (15 mg, 23 μmol, 43%).

MS (ESI): m/z (+)=640.6.

¹H NMR (400 MHz, MeOD) δ 9.06 (s, 1H), 6.87 (d, J=2.4 Hz, 1H), 6.71 (d, J=2.4 Hz, 1H), 5.51-5.30 (m, 1H), 4.86-4.75 (m, 1H), 4.67-4.58 (m, 1H), 4.51-4.32 (m, 2H), 3.66-3.32 (m, 6H), 3.28-2.99 (m, 3H), 2.71-2.60 (m, 1H), 2.53-2.06 (m, 7H), 2.06-1.64 (m, 6H), 1.64-1.16 (m, 6H).

### Example 30. 3⁵-amino-2⁶,3³-dichloro-2⁸-fluoro-2²-(((2R,7aS)-2-fluorotetrahydro-1H-pyrrolizin-7a(5H)-yl)methoxy)-9-aza-2(4,7)-quinazolina-1(1,3)-piperidina-3(1,2)-benzenacycloundecaphan-8-one (Compound 155a)

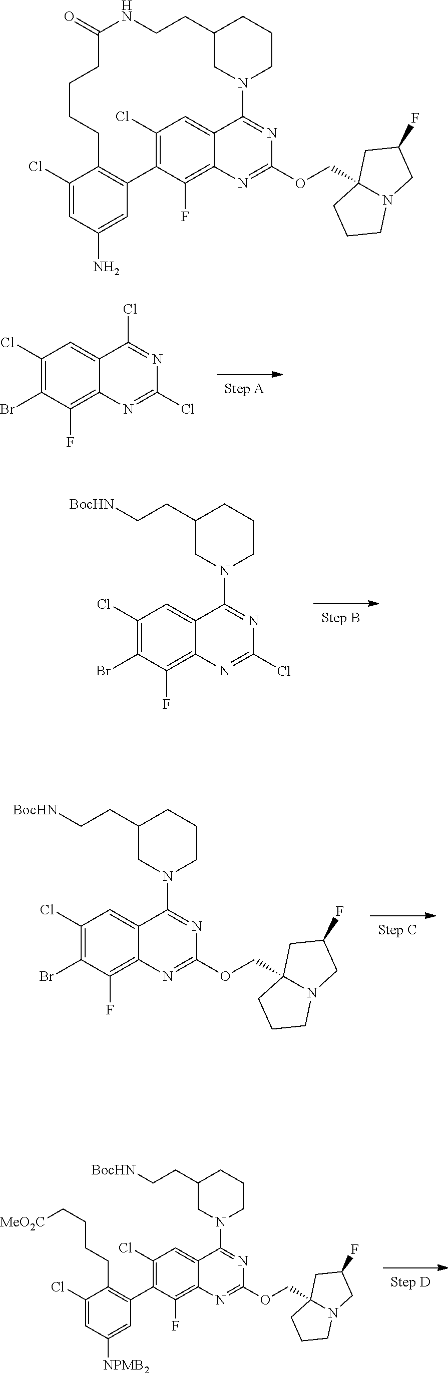

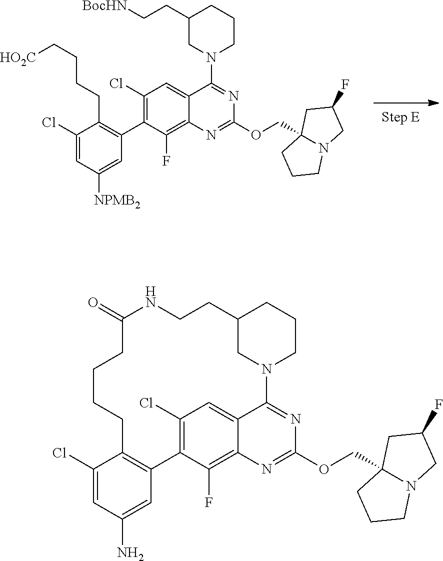

### Step A: Synthesis of tert-butyl (2-(1-(7-bromo-2,6-dichloro-8-fluoroquinazolin-4-yl)piperidin-3-yl)ethyl)carbamate

A solution of 7-bromo-2,4,6-trichloro-8-fluoroquinazoline (1500 mg, 1 equiv., 4.541 mmol) and DIPEA (2.347 g, 3.16 mL, 4 equiv., 18.16 mmol) in THF (20 mL) at room temperature was treated with tert-butyl (2-(piperidin-3-yl)ethyl)carbamate (1.037 g, 1 equiv., 4.541 mmol). The mixture was stirred at 50° C. for 16 hours. The reaction mixture was concentrated onto celite and was purified by flash column chromatography (ethyl acetate/heptane) to afford tert-butyl (2-(1-(7-bromo-2,6-dichloro-8-fluoroquinazolin-4-yl)piperidin-3-yl)ethyl)carbamate (3.87 g, 7.41 mmol, 81.6%).

MS (ESI): m/z (+)=521.3

### Step B: Synthesis of tert-butyl (2-(1-(7-bromo-6-chloro-8-fluoro-2-(((2R,7aS)-2-fluorotetrahydro-1H-pyrrolizin-7a(5H)-yl)methoxy)quinazolin-4-yl)piperidin-3-yl)ethyl)carbamate

A solution of tert-butyl (2-(1-(7-bromo-2,6-dichloro-8-fluoroquinazolin-4-yl)piperidin-3-yl)ethyl)carbamate (500 mg, 1 equiv., 957 μmol), ((2R,7aS)-2-fluorotetrahydro-1H-pyrrolizin-7a(5H)-yl)methanol (305 mg, 2 equiv., 1.91 mmol), cataCXium A Pd G3 (69.7 mg, 0.1 equiv., 95.7 μmol), and Cs₂CO₃ (936 mg, 3 equiv., 2.87 mmol) in 1,4-dioxane (5 mL) was degassed with nitrogen and was warmed at 80° C. for 3 hours. The reaction mixture was concentrated and was purified by reverse-phase flash column chromatography using a gradient of 10-100% B (A: water+0.1% formic acid; B: acetonitrile+0.1% formic acid) to afford tert-butyl (2-(1-(7-bromo-6-chloro-8-fluoro-2-(((2R,7aS)-2-fluorotetrahydro-1H-pyrrolizin-7a(5H)-yl)methoxy)quinazolin-4-yl)piperidin-3-yl)ethyl)carbamate (618 mg, 957 μmol, 100%).

MS (ESI): m/z (+)=644.3

### Step C: Synthesis of methyl 5-(4-(bis(4-methoxybenzyl)amino)-2-(4-(3-(2-((tert-butoxycarbonyl)amino)ethyl)piperidin-1-yl)-6-chloro-8-fluoro-2-(((2R,7aS)-2-fluorotetrahydro-1H-pyrrolizin-7a(5H)-yl)methoxy)quinazolin-7-yl)-6-chlorophenyl)pentanoate

A solution of tert-butyl (2-(1-(7-bromo-6-chloro-8-fluoro-2-(((2R,7aS)-2-fluorotetrahydro-1H-pyrrolizin-7a(5H)-yl)methoxy)quinazolin-4-yl)piperidin-3-yl)ethyl)carbamate (387 mg, 1 equiv., 600 μmol), methyl 5-(4-(bis(4-methoxybenzyl)amino)-2-chloro-6-(4,4,5,5-tetramethyl-1,3,2-dioxaborolan-2-yl)phenyl)pentanoate (365 mg, 1 equiv., 600 μmol), XPhos Pd G3 (50.8 mg, 0.1 equiv., 60.0 μmol), and Potassium phosphate, tribasic (382 mg, 1.20 mL, 1.5 M in water, 3 equiv., 1.80 mmol) in THF (10 mL) was degassed with nitrogen and was warmed at 80° C. for 1 hour. The reaction mixture was concentrated onto celite and was purified by reverse-phase flash column chromatography using a gradient of 10-100% B (A: water+0.1% formic acid; B: acetonitrile+0.1% formic acid) to afford methyl 5-(4-(bis(4-methoxybenzyl)amino)-2-(4-(3-(2-((tert-butoxycarbonyl)amino)ethyl)piperidin-1-yl)-6-chloro-8-fluoro-2-(((2R,7aS)-2-fluorotetrahydro-1H-pyrrolizin-7a(5H)-yl)methoxy)quinazolin-7-yl)-6-chlorophenyl)pentanoate (413 mg, 395 μmol, 65.8%).

MS (ESI): m/z (+)=1045.7

### Step D: Synthesis of 5-(4-(bis(4-methoxybenzyl)amino)-2-(4-(3-(2-((tert-butoxycarbonyl)amino)ethyl)piperidin-1-yl)-6-chloro-8-fluoro-2-(((2R,7aS)-2-fluorotetrahydro-1H-pyrrolizin-7a(5H)-yl)methoxy)quinazolin-7-yl)-6-chlorophenyl)pentanoic acid

A solution of methyl 5-(4-(bis(4-methoxybenzyl)amino)-2-(4-(3-(2-((tert-butoxycarbonyl)amino)ethyl)piperidin-1-yl)-6-chloro-8-fluoro-2-(((2R,7aS)-2-fluorotetrahydro-1H-pyrrolizin-7a(5H)-yl)methoxy)quinazolin-7-yl)-6-chlorophenyl)pentanoate (413 mg, 1 equiv., 395 μmol) in THF (8 mL), MeOH (2 mL), and water (2 mL) was treated with LiOH·H₂O (166 mg, 10 equiv., 3.95 mmol). The mixture was stirred for 4 hours. The reaction mixture was concentrated and was purified by reverse-phase flash column chromatography using a gradient of 10-100% B (A: water+0.1% formic acid; B: acetonitrile+0.1% formic acid) to afford 5-(4-(bis(4-methoxybenzyl)amino)-2-(4-(3-(2-((tert-butoxycarbonyl)amino)ethyl)piperidin-1-yl)-6-chloro-8-fluoro-2-(((2R,7aS)-2-fluorotetrahydro-1H-pyrrolizin-7a(5H)-yl)methoxy)quinazolin-7-yl)-6-chlorophenyl)pentanoic acid (379 mg, 367 μmol, 93.0%).

MS (ESI): m/z (+)=1031.7

### Step E: Synthesis of 3⁵-amino-2⁶,3³-dichloro-2⁸-fluoro-2²-(((2R,7aS)-2-fluorotetrahydro-1H-pyrrolizin-7a(5H)-yl)methoxy)-9-aza-2(4,7)-quinazolina-1(1,3)-piperidina-3(1,2)-benzenacycloundecaphan-8-one

A solution of 5-(4-(bis(4-methoxybenzyl)amino)-2-(4-(3-(2-((tert-butoxycarbonyl)amino)ethyl)piperidin-1-yl)-6-chloro-8-fluoro-2-(((2R,7aS)-2-fluorotetrahydro-1H-pyrrolizin-7a(5H)-yl)methoxy)quinazolin-7-yl)-6-chlorophenyl)pentanoic acid (379 mg, 1 equiv., 367 μmol) in DCM (1 mL) was treated with HCl (669 mg, 4.59 mL, 4 M, 50 equiv., 18.4 mmol). The mixture was stirred at room temperature for 10 minutes. The volatiles were removed under a stream of nitrogen and the residue was further dried under vacuum. The resulting residue was taken up in DMF (4 mL) and was treated with HATU (168 mg, 1.2 equiv., 441 μmol) and DIPEA (237 mg, 320 μL, 5 equiv., 1.84 mmol). The mixture was stirred at room temperature for 20 minutes. The reaction mixture was diluted with EtOAc, washed with water and brine, dried (Na₂SO₄), filtered, and concentrated. The resulting residue was treated with TFA (4.19 g, 2.83 mL, 100 equiv., 36.7 mmol). The mixture was stirred at 50° C. for 60 minutes. The volatiles were removed under a stream of nitrogen and the resulting residue was purified by HPLC (Method A) to afford 3⁵-amino-2⁶,3³-dichloro-2⁸-fluoro-2²-(((2R,7aS)-2-fluorotetrahydro-1H-pyrrolizin-7a(5H)-yl)methoxy)-9-aza-2(4,7)-quinazolina-1(1,3)-piperidina-3(1,2)-benzenacycloundecaphan-8-one (92 mg, 0.14 mmol, 37%).

MS (ESI): m/z (+)=673.1

¹H NMR (400 MHz, MeOD) δ 7.91 (s, 1H), 6.84 (d, J=2.3 Hz, 1H), 6.50 (d, J=2.3 Hz, 1H), 5.46-5.22 (m, 1H), 4.80-4.68 (m, 1H), 4.57-4.47 (m, 1H), 4.40-4.19 (m, 3H), 3.49-3.32 (m, 4H), 3.17-2.92 (m, 3H), 2.66-2.55 (m, 1H), 2.45-2.09 (m, 4H), 2.11-1.67 (m, 5H), 1.67-1.13 (m, 12H).

### Example 31. 3⁵-amino-3³-chloro-2⁸-fluoro-2²-(((2R,7aS)-2-fluorotetrahydro-1H-pyrrolizin-7a(5H)-yl)methoxy)-13-hydroxy-9-aza-2(4,7)-pyrido[4,3-d]pyrimidina-1(1,3)-piperidina-3(1,2)-benzenacycloundecaphan-8-one (Compound 156a)

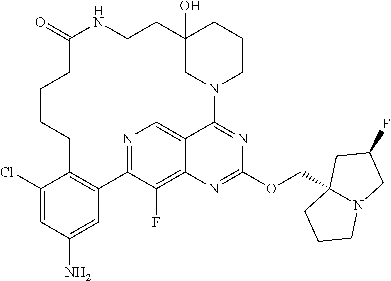 [SMILES: Nc1cc(Cl)c2c(c1)-c1ncc3c(nc(OC[C@@]45CCCN4C[C@H](F)C5)nc3c1F)N1CCCC(O)(CCNC(=O)CCCC2)C1]

3⁵-amino-3³-chloro-2⁸-fluoro-2²-(((2R,7aS)-2-fluorotetrahydro-1H-pyrrolizin-7a(5H)-yl)methoxy)-13-hydroxy-9-aza-2(4,7)-pyrido[4,3-d]pyrimidina-1(1,3)-piperidina-3(1,2)-benzenacycloundecaphan-8-one was prepared in an analogous manner to Example 29 by using tert-butyl (2-(1-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3-hydroxypiperidin-3-yl)ethyl)carbamate in Step A.

MS (ESI): m/z (+)=656.5

¹H NMR (400 MHz, MeOD) δ 9.22 (s, 1H), 6.86 (d, J=2.4 Hz, 1H), 6.68 (d, J=2.4 Hz, 1H), 5.65-5.42 (m, 1H), 4.79-4.46 (m, 3H), 4.01-3.60 (m, 4H), 3.57-3.35 (m, 2H), 3.28-3.13 (m, 1H), 2.72-1.63 (m, 17H), 1.54-1.05 (m, 6H).

### Example 32. 3⁵-amino-2⁸-fluoro-2²-(((2R,7aS)-2-fluorotetrahydro-1H-pyrrolizin-7a(5H)-yl)methoxy)-1³-hydroxy-9-aza-2(4,7)-pyrido[4,3-d]pyrimidina-1(1,3)-piperidina-3(1,2)-benzenacycloundecaphan-8-one (Compound 157a)

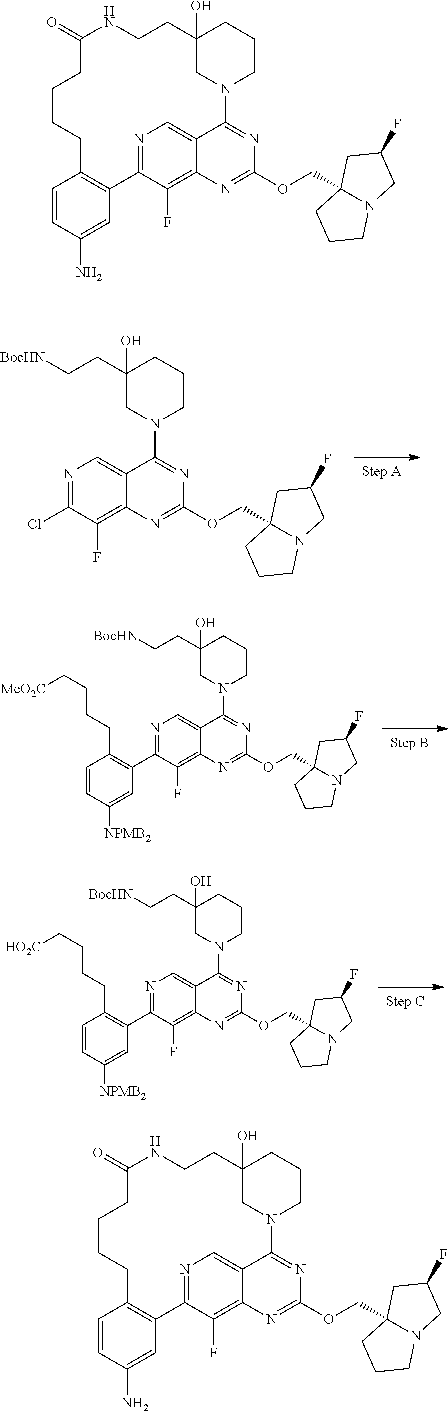

### Step A: Synthesis of methyl 5-(4-(bis(4-methoxybenzyl)amino)-2-(4-(3-(2-((tert-butoxycarbonyl)amino)ethyl)-3-hydroxypiperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)phenyl)pentanoate

A solution of tert-butyl (2-(1-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3-hydroxypiperidin-3-yl)ethyl)carbamate (200 mg, 1 equiv., 343 μmol), methyl 5-(4-(bis(4-methoxybenzyl)amino)-2-chloro-6-(4,4,5,5-tetramethyl-1,3,2-dioxaborolan-2-yl)phenyl)pentanoate (209 mg, 1.0 equiv., 343 μmol), Cs₂CO₃ (335 mg, 3 equiv., 1.03 mmol), and cataCXium A Pd G3 (25.0 mg, 0.1 equiv., 34.3 μmol) in 1,4-dioxane (8 mL) and water (1 mL) was warmed at 80° C. for 30 min. The reaction mixture was concentrated onto celite and purified by flash column chromatography (0-100% 3:1 EtOAc:EtOH/DCM) to afford methyl 5-(4-(bis(4-methoxybenzyl)amino)-2-(4-(3-(2-((tert-butoxycarbonyl)amino)ethyl)-3-hydroxypiperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)phenyl)pentanoate (47 mg, 47 μmol, 14%).

MS (ESI): m/z (+)=994.7.

### Step B: Synthesis of 5-(4-(bis(4-methoxybenzyl)amino)-2-(4-(3-(2-((tert-butoxycarbonyl)amino)ethyl)-3-hydroxypiperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)phenyl)pentanoic acid

A solution of methyl 5-(4-(bis(4-methoxybenzyl)amino)-2-(4-(3-(2-((tert-butoxycarbonyl)amino)ethyl)-3-hydroxypiperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)phenyl)pentanoate (47 mg, 1 equiv., 47 μmol) in THF (2 mL) and water (0.5 mL) was treated with LiOH·H₂O (20 mg, 10 equiv., 0.47 mmol). The mixture was stirred for 1 hour. The reaction mixture was warmed at 40° C. for 2 hours, concentrated, and purified by reverse-phase flash column chromatography using a gradient of 10-100% B (A: water+0.1% formic acid; B: acetonitrile+0.1% formic acid) to afford 5-(4-(bis(4-methoxybenzyl)amino)-2-(4-(3-(2-((tert-butoxycarbonyl)amino)ethyl)-3-hydroxypiperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)phenyl)pentanoic acid (33 mg, 34 μmol, 71%).

MS (ESI): m/z (+)=980.7.

### Step C: Synthesis of 3⁵-amino-2⁸-fluoro-2²-(((2R,7aS)-2-fluorotetrahydro-1H-pyrrolizin-7a(5H)-yl)methoxy)-1³-hydroxy-9-aza-2(4,7)-pyrido[4,3-d]pyrimidina-1(1,3)-piperidina-3(1,2)-benzenacycloundecaphan-8-one

A solution of 5-(4-(bis(4-methoxybenzyl)amino)-2-(4-(3-(2-((tert-butoxycarbonyl)amino)ethyl)-3-hydroxypiperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)phenyl)pentanoic acid (33 mg, 1 equiv., 34 μmol) in DCM (1 mL) was treated with HCl (0.12 g, 0.84 mL, 4 M, 100 equiv., 3.4 mmol). The mixture was stirred at room temperature for 20 minutes. The volatiles were removed under a stream of nitrogen, and the residue was further dried under vacuum. The resulting residue was taken up in DMF (4 mL) and treated with HATU (15 mg, 1.2 equiv., 40 μmol) and DIPEA (22 mg, 29 μL, 5 equiv., 0.17 mmol). The mixture was stirred at room temperature for 15 minutes. The reaction mixture was diluted with EtOAc, washed with water and brine, dried (Na₂SO₄), filtered, and concentrated. The resulting residue was treated with TFA (0.38 g, 0.26 mL, 100 equiv., 3.4 mmol). The mixture was stirred at 50° C. for 30 minutes followed by warming at 65° C. for 16 hours. The volatiles were removed under a stream of nitrogen, and the resulting residue was purified by HPLC (Method A) to afford 3⁵-amino-2⁸-fluoro-2²-(((2R,7aS)-2-fluorotetrahydro-1H-pyrrolizin-7a(5H)-yl)methoxy)-1³-hydroxy-9-aza-2(4,7)-pyrido[4,3-d]pyrimidina-1(1,3)-piperidina-3(1,2)-benzenacycloundecaphan-8-one (7.9 mg, 13 μmol, 38%).

MS (ESI): m/z (+)=622.1.

¹H NMR (400 MHz, MeOD) δ 9.19 (d, J=3.9 Hz, 1H), 7.09 (d, J=8.0 Hz, 1H), 6.85-6.80 (m, 2H), 5.67-5.45 (m, 1H), 4.72-4.58 (m, 3H), 4.56-4.45 (m, 1H), 4.06-3.69 (m, 4H), 3.50-3.38 (m, 2H), 3.23-3.10 (m, 1H), 2.76-2.03 (m, 12H), 1.92-1.64 (m, 5H), 1.47-1.06 (m, 5H).

### Example 33. 3⁵-amino-2⁶,3³-dichloro-2⁸-fluoro-2²-(((2R,7aS)-2-fluorotetrahydro-1H-pyrrolizin-7a(5H)-yl)methoxy)-1³-hydroxy-9-aza-2(4,7)-quinazolina-1(1,3)-piperidina-3(1,2)-benzenacycloundecaphan-8-one (Compound 158a)

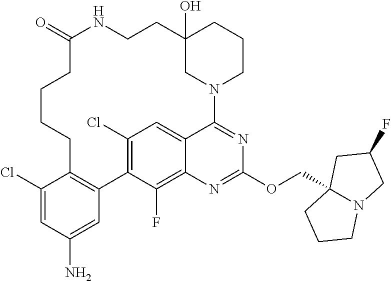 [SMILES: Nc1cc(Cl)c2c(c1)-c1c(Cl)cc3c(nc(OC[C@@]45CCCN4C[C@H](F)C5)nc3c1F)N1CCCC(O)(CCNC(=O)CCCC2)C1]

3⁵-amino-2⁶,3³-dichloro-2⁸-fluoro-2²-(((2R,7aS)-2-fluorotetrahydro-1H-pyrrolizin-7a(5H)-yl)methoxy)-1³-hydroxy-9-aza-2(4,7)-quinazolina-1(1,3)-piperidina-3(1,2)-benzenacycloundecaphan-8-one was prepared in an analogous manner to Example 30 by using tert-butyl (2-(3-hydroxypiperidin-3-yl)ethyl)carbamate in Step A.

LCMS: Retention time=2.08 min, m/z (+)=689.1.

### Example 34. 3⁵-amino-2⁸,3⁴-difluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacycloundecaphan-8-one (Compound 159a)

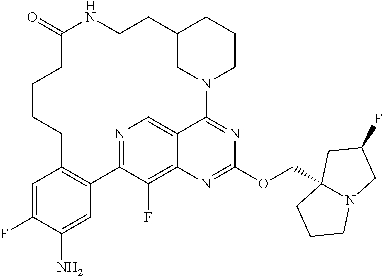 [SMILES: Nc1cc2c(cc1F)CCCCC(=O)NCCC1CCCN(C1)c1nc(OC[C@@]34CCCN3C[C@H](F)C4)nc3c(F)c-2ncc13]

3⁵-amino-2⁸,3⁴-difluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacycloundecaphan-8-one was prepared in an analogous manner to Example 29 by using methyl 5-(4-amino-5-fluoro-2-(4,4,5,5-tetramethyl-1,3,2-dioxaborolan-2-yl)phenyl)pentanoate in Step A.

MS (ESI): m/z (+)=624.1.

¹H NMR (400 MHz, MeOD) δ 9.05 (s, 1H), 6.98-6.92 (m, 2H), 5.54-5.34 (m, 1H), 4.83-4.73 (m, 1H), 4.64-4.55 (m, 1H), 4.53-4.36 (m, 2H), 3.69-3.34 (m, 4H), 3.28-3.16 (m, 1H), 3.08-2.96 (m, 1H), 2.56-1.10 (m, 23H).

### Example 35. 3⁵-amino-2⁸,3⁶-difluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacycloundecaphan-8-one (Compound 160a)

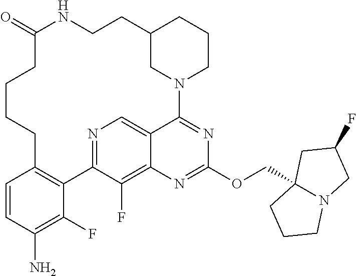 [SMILES: Nc1ccc2c(c1F)-c1ncc3c(nc(OC[C@@]45CCCN4C[C@H](F)C5)nc3c1F)N1CCCC(CCNC(=O)CCCC2)C1]

3⁵-amino-2⁸,3⁶-difluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacycloundecaphan-8-one was prepared in an analogous manner to Example 29 by using methyl 5-(4-amino-3-fluoro-2-(4,4,5,5-tetramethyl-1,3,2-dioxaborolan-2-yl)phenyl)pentanoate in Step A.

MS (ESI): m/z (+)=624.2.

¹H NMR (400 MHz, MeOD) δ 9.13 (s, 1H), 6.99-6.85 (m, 2H), 5.53-5.31 (m, 1H), 5.05-4.94 (m, 1H), 4.73-4.64 (m, 1H), 4.56-4.34 (m, 3H), 3.69-3.34 (m, 5H), 3.28-2.99 (m, 2H), 2.58-1.74 (m, 13H), 1.73-1.18 (m, 8H).

### Example 36. 3⁵-amino-2⁸,3⁴-difluoro-2²-(((2R,7aS)-2-fluorotetrahydro-1H-pyrrolizin-7a(5H)-yl)methoxy)-1³-hydroxy-9-aza-2(4,7)-pyrido[4,3-d]pyrimidina-1(1,3)-piperidina-3(1,2)-benzenacycloundecaphan-8-one (Compound 161a)

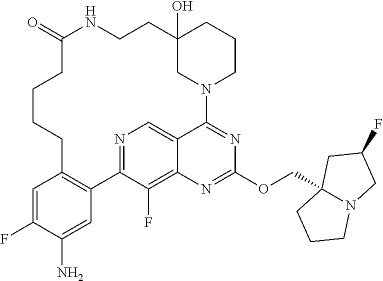 [SMILES: Nc1cc2c(cc1F)CCCCC(=O)NCCC1(O)CCCN(C1)c1nc(OC[C@@]34CCCN3C[C@H](F)C4)nc3c(F)c-2ncc13]

3⁵-amino-2⁸,3⁴-difluoro-2²-(((2R,7aS)-2-fluorotetrahydro-1H-pyrrolizin-7a(5H)-yl)methoxy)-1³-hydroxy-9-aza-2(4,7)-pyrido[4,3-d]pyrimidina-1(1,3)-piperidina-3(1,2)-benzenacycloundecaphan-8-one was prepared in an analogous manner to Example 32 by using methyl 5-(4-amino-5-fluoro-2-(4,4,5,5-tetramethyl-1,3,2-dioxaborolan-2-yl)phenyl)pentanoate in Step A.

MS (ESI): m/z (+)=640.1.

¹H NMR (400 MHz, MeOD) δ 9.19 (d, J=2.9 Hz, 1H), 6.96 (d, J=12.1 Hz, 1H), 6.92 (d, J=9.0 Hz, 1H), 5.59-5.41 (m, 1H), 4.73-4.46 (m, 4H), 3.87-3.63 (m, 5H), 3.49-3.33 (m, 1H), 3.25-3.10 (m, 1H), 2.65-1.98 (m, 10H), 1.98-1.63 (m, 8H), 1.48-1.08 (m, 4H).

### Example 37. Synthesis of 2⁸,3⁷-difluoro-2²-(((2R,7aS)-2-fluorotetrahydro-1H-pyrrolizin-7a(5H)-yl)methoxy)-8-aza-2(4,7)-pyrido[4,3-d]pyrimidina-1(1,3)-piperidina-3(1,8)-naphthalenacyclodecaphan-7-one (Compound 170a)

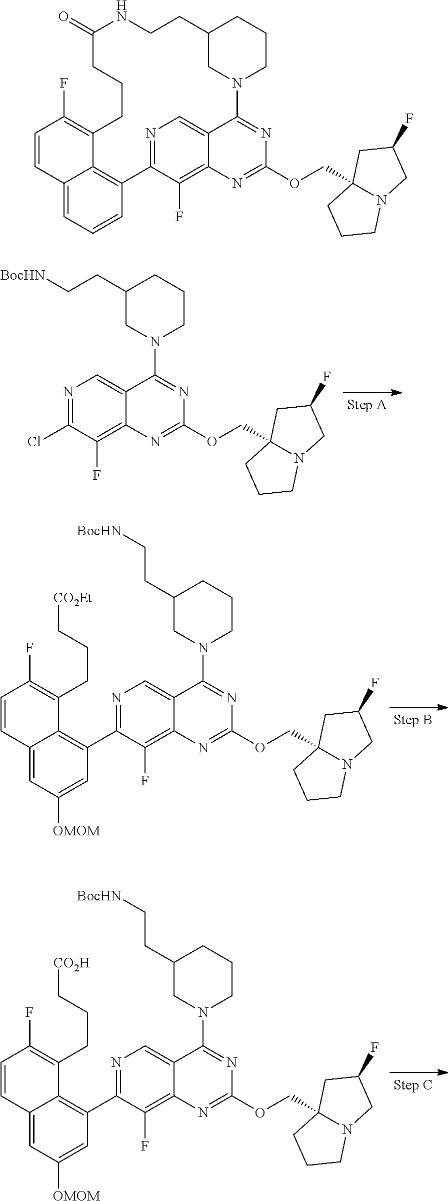

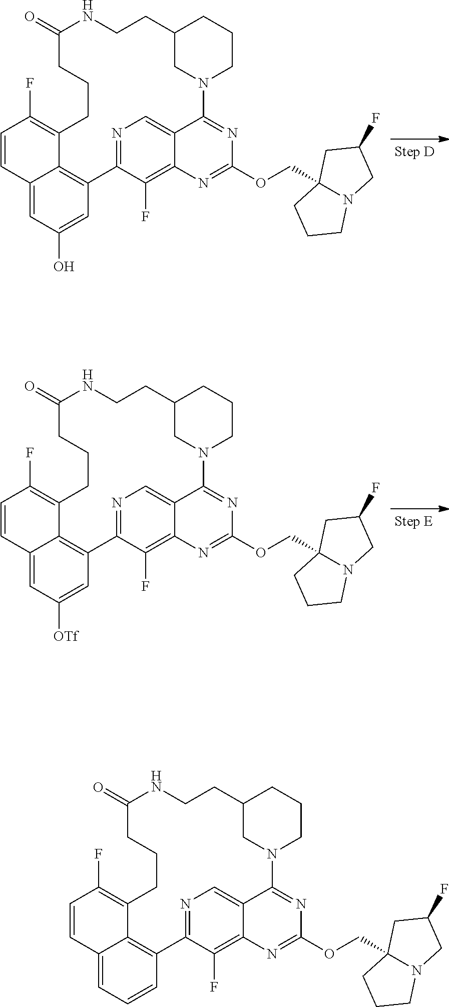 [SMILES: O=C1CCCc2c(F)ccc3cc(O)cc(c23)-c2ncc3c(nc(OC[C@@]45CCCN4C[C@H](F)C5)nc3c2F)N2CCCC(CCN1)C2 ; O=C1CCCc2c(F)ccc3cc(OS(=O)(=O)C(F)(F)F)cc(c23)-c2ncc3c(nc(OC[C@@]45CCCN4C[C@H](F)C5)nc3c2F)N2CCCC(CCN1)C2 ; O=C1CCCc2c(F)ccc3cccc(c23)-c2ncc3c(nc(OC[C@@]45CCCN4C[C@H](F)C5)nc3c2F)N2CCCC(CCN1)C2]

### Step A: Synthesis of ethyl 4-(8-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoate

A solution of tert-butyl (2-(1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)ethyl)carbamate (200 mg, 353 μmol), ethyl 4-(2-fluoro-6-(methoxymethoxy)-8-(4,4,5,5-tetramethyl-1,3,2-dioxaborolan-2-yl)naphthalen-1-yl)butanoate (157 mg, 353 μmol), Cs₂CO₃ (345 mg, 1.06 mmol), and cataCXium A Pd G3 (25.7 mg, 35.3 μmol) in 1,4-dioxane (4 mL) and water (0.5 mL) was warmed at 80° C. for 2 hours. The reaction mixture was concentrated onto celite and purified by reverse-phase flash column chromatography using a gradient of 10-100% B (A: water+0.1% formic acid; B: acetonitrile+0.1% formic acid) to afford ethyl 4-(8-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoate (122 mg, 143 μmol, 40.6%).

MS (ESI): m/z (+)=851.5.

### Step B: Synthesis of 4-(8-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoic acid

A solution of ethyl 4-(8-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoate (175 mg, 206 μmol) in THF (5 mL) and water (1.5 mL) was treated with LiOH·H₂O (129 mg, 3.08 mmol). The mixture was stirred at room temperature for 20 hours. The reaction mixture was concentrated and purified by reverse-phase flash column chromatography using a gradient of 10-100% B (A: water+0.1% formic acid; B: acetonitrile+0.1% formic acid) to afford 4-(8-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoic acid (123 mg, 149 μmol, 72.7%).

MS (ESI): m/z (+)=823.5.

### Step C: Synthesis of 2⁸,3⁷-difluoro-2²-(((2R,7aS)-2-fluorotetrahydro-1H-pyrrolizin-7a(5H)-yl)methoxy)-3³-hydroxy-8-aza-2(4,7)-pyrido[4,3-d]pyrimidina-1(1,3)-piperidina-3(1,8)-naphthalenacyclodecaphan-7-one

A solution of 4-(8-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoic acid (55 mg, 67 μmol) in DCM (1 mL) was treated with hydrogen chloride (0.84 mL, 4 M, 3.3 mmol). The mixture was stirred at room temperature for 30 minutes. The volatiles were removed under a stream of nitrogen, and the residue was further dried under vacuum. The resulting residue was taken up in DMF (5 mL) and treated with HATU (30 mg, 80 μmol) and DIPEA (43 mg, 58 μL, 0.33 mmol). The mixture was stirred at room temperature for 30 minutes. The reaction mixture was purified by reverse-phase flash column chromatography using a gradient of 10-100% B (A: water+0.1% formic acid; B: acetonitrile+0.1% formic acid) to afford 2⁸,3⁷-difluoro-2²-(((2R,7aS)-2-fluorotetrahydro-1H-pyrrolizin-7a(5H)-yl)methoxy)-3³-hydroxy-8-aza-2(4,7)-pyrido[4,3-d]pyrimidina-1(1,3)-piperidina-3(1,8)-naphthalenacyclodecaphan-7-one (25 mg, 38 μmol, 57%).

MS (ESI): m/z (+)=661.3.

### Step D: Synthesis of 2⁸,3⁷-difluoro-2²-(((2R,7aS)-2-fluorotetrahydro-1H-pyrrolizin-7a(5H)-yl)methoxy)-7-oxo-8-aza-2(4,7)-pyrido[4,3-d]pyrimidina-1(1,3)-piperidina-3(1,8)-naphthalenacyclodecaphane-3³-yl trifluoromethanesulfonate

A solution of 2⁸,3⁷-difluoro-2²-(((2R,7aS)-2-fluorotetrahydro-1H-pyrrolizin-7a(5H)-yl)methoxy)-3³-hydroxy-8-aza-2(4,7)-pyrido[4,3-d]pyrimidina-1(1,3)-piperidina-3(1,8)-naphthalenacyclodecaphan-7-one (25 mg, 38 μmol) and DIPEA (15 mg, 20 μL, 0.11 mmol) in DCM (1 mL) at room temperature was treated with trifluoromethanesulfonic anhydride (16 mg, 9.6 μL, 57 μmol). The mixture was stirred at room temperature for 30 min. The reaction mixture was concentrated and was purified by reverse-phase flash column chromatography using a gradient of 10-100% B (A: water+0.1% formic acid; B: acetonitrile+0.1% formic acid) to afford 2⁸,3⁷-difluoro-2²-(((2R,7aS)-2-fluorotetrahydro-1H-pyrrolizin-7a(5H)-yl)methoxy)-7-oxo-8-aza-2(4,7)-pyrido[4,3-d]pyrimidina-1(1,3)-piperidina-3(1,8)-naphthalenacyclodecaphane-3³-yl trifluoromethanesulfonate (8.7 mg, 11 μmol, 29%).

MS (ESI): m/z (+)=793.4.

### Step E: Synthesis of 2⁸,3⁷-difluoro-2²-(((2R,7aS)-2-fluorotetrahydro-1H-pyrrolizin-7a(5H)-yl)methoxy)-8-aza-2(4,7)-pyrido[4,3-d]pyrimidina-1(1,3)-piperidina-3(1,8)-naphthalenacyclodecaphan-7-one

A solution of 2⁸,3⁷-difluoro-2²-(((2R,7aS)-2-fluorotetrahydro-1H-pyrrolizin-7a(5H)-yl)methoxy)-7-oxo-8-aza-2(4,7)-pyrido[4,3-d]pyrimidina-1(1,3)-piperidina-3(1,8)-naphthalenacyclodecaphane-3³-yl trifluoromethanesulfonate (14.5 mg, 18.3 μmol), tert-butyl carbamate (5.36 mg, 45.7 μmol), BrettPhos-Pd-G3 (3.32 mg, 3.66 μmol), and Cs₂CO₃ (17.9 mg, 54.9 μmol) in DMF (1 mL) was degassed with nitrogen. The mixture was stirred at 80° C. for 7 hours. The reaction mixture was filtered and purified by HPLC (Method A) to afford 2⁸,3⁷-difluoro-2²-(((2R,7aS)-2-fluorotetrahydro-1H-pyrrolizin-7a(5H)-yl)methoxy)-8-aza-2(4,7)-pyrido[4,3-d]pyrimidina-1(1,3)-piperidina-3(1,8)-naphthalenacyclodecaphan-7-one (3.6 mg, 5.6 μmol, 31%).

MS (ESI): m/z (+)=645.4.

¹H NMR (400 MHz, MeOD) δ 9.02 (s, 1H), 8.03-7.95 (m, 1H), 7.91-7.85 (m, 1H), 7.54-7.45 (m, 1H), 7.45-7.40 (m, 1H), 7.35-7.28 (m, 1H), 5.43-5.23 (m, 1H), 4.83-4.78 (m, 1H), 4.49-4.24 (m, 2H), 3.56-3.30 (m, 4H), 3.18-3.05 (m, 2H), 2.81-2.65 (m, 2H), 2.41-1.22 (m, 20H).

### Example 38. Synthesis of 3⁵-amino-3³-chloro-2⁸-fluoro-2²-(((S)-1-methylpyrrolidin-2-yl)methoxy)-9-aza-2(4,7)-pyrido[4,3-d]pyrimidina-1(1,3)-piperidina-3(1,2)-benzenacycloundecaphan-8-one (Compound 171a)

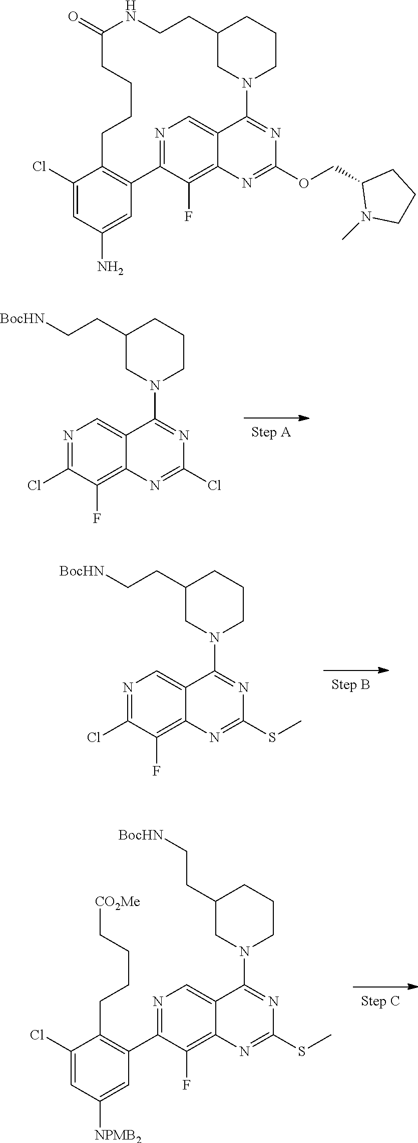

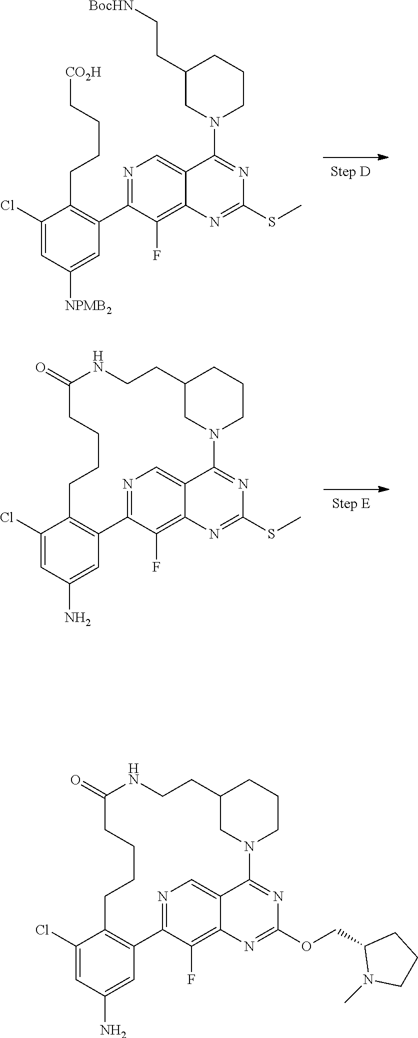

### Step A: Synthesis of tert-butyl (2-(1-(7-chloro-8-fluoro-2-(methylthio)pyrido[4,3-d]pyrimidin-4-yl)piperidin-3-yl)ethyl)carbamate

A solution of tert-butyl (2-(1-(2,7-dichloro-8-fluoropyrido[4,3-d]pyrimidin-4-yl)piperidin-3-yl)ethyl)carbamate (670 mg, 1.51 mmol) in THF (7.5 mL) was cooled to 0° C. Sodium methanethiolate (148 mg, 2.11 mmol) was added in one portion, and the mixture was warmed to room temperature. The mixture was stirred for 2 hours. The reaction mixture was diluted with water and EtOAc, washed with brine, dried (Na₂SO₄), filtered, and concentrated in vacuo. The resulting residue was purified by flash column chromatography (ethyl acetate/heptane) to afford tert-butyl (2-(1-(7-chloro-8-fluoro-2-(methylthio)pyrido[4,3-d]pyrimidin-4-yl)piperidin-3-yl)ethyl)carbamate (596 mg, 1.31 mmol, 86.7%).

MS (ESI): m/z (+)=456.2.

### Step B: Synthesis of methyl 5-(4-(bis(4-methoxybenzyl)amino)-2-(4-(3-(2-((tert-butoxycarbonyl)amino)ethyl)piperidin-1-yl)-8-fluoro-2-(methylthio)pyrido[4,3-d]pyrimidin-7-yl)-6-chlorophenyl)pentanoate

A solution of tert-butyl (2-(1-(7-chloro-8-fluoro-2-(methylthio)pyrido[4,3-d]pyrimidin-4-yl)piperidin-3-yl)ethyl)carbamate (327 mg, 717 μmol), methyl 5-(4-(bis(4-methoxybenzyl)amino)-2-chloro-6-(4,4,5,5-tetramethyl-1,3,2-dioxaborolan-2-yl)phenyl)pentanoate (523 mg, 861 μmol), Cs₂CO₃ (701 mg, 2.15 mmol), and cataCXium A Pd G3 (52.2 mg, 71.7 μmol) in 1,4-dioxane (6 mL) and water (1 mL) was degassed with nitrogen and warmed at 80° C. for 2 hours. The reaction mixture was concentrated onto celite and purified by flash column chromatography (ethyl acetate/heptane) to afford methyl 5-(4-(bis(4-methoxybenzyl)amino)-2-(4-(3-(2-((tert-butoxycarbonyl)amino)ethyl)piperidin-1-yl)-8-fluoro-2-(methylthio)pyrido[4,3-d]pyrimidin-7-yl)-6-chlorophenyl)pentanoate (421 mg, 467 μmol, 65.1%).

MS (ESI): m/z (+)=901.4.

### Step C: Synthesis of 5-(4-(bis(4-methoxybenzyl)amino)-2-(4-(3-(2-((tert-butoxycarbonyl)amino)ethyl)piperidin-1-yl)-8-fluoro-2-(methylthio)pyrido[4,3-d]pyrimidin-7-yl)-6-chlorophenyl)pentanoic acid

A solution of methyl 5-(4-(bis(4-methoxybenzyl)amino)-2-(4-(3-(2-((tert-butoxycarbonyl)amino)ethyl)piperidin-1-yl)-8-fluoro-2-(methylthio)pyrido[4,3-d]pyrimidin-7-yl)-6-chlorophenyl)pentanoate (421 mg, 467 μmol) in THF (6 mL) and water (1.5 mL) was treated with LiOH·H₂O (196 mg, 4.67 mmol). The mixture was stirred at room temperature for 16 hours. LiOH·H₂O (196 mg, 4.67 mmol) was added, and the reaction mixture was stirred at room temperature for 24 hours. The reaction mixture was diluted with EtOAc, washed with brine, dried (Na₂SO₄), filtered, and concentrated to afford crude 5-(4-(bis(4-methoxybenzyl)amino)-2-(4-(3-(2-((tert-butoxycarbonyl)amino)ethyl)piperidin-1-yl)-8-fluoro-2-(methylthio)pyrido[4,3-d]pyrimidin-7-yl)-6-chlorophenyl)pentanoic acid (410 mg, 0.37 mmol, 79%), which was used directly in the next step without purification.

MS (ESI): m/z (+)=887.6.

### Step D: Synthesis of 3⁵-(bis(4-methoxybenzyl)amino)-3³-chloro-2⁸-fluoro-2²-(methylthio)-9-aza-2(4,7)-pyrido[4,3-d]pyrimidina-1(1,3)-piperidina-3(1,2)-benzenacycloundecaphan-8-one

A solution of 5-(4-(bis(4-methoxybenzyl)amino)-2-(4-(3-(2-((tert-butoxycarbonyl)amino)ethyl)piperidin-1-yl)-8-fluoro-2-(methylthio)pyrido[4,3-d]pyrimidin-7-yl)-6-chlorophenyl)pentanoic acid (410 mg, 462 μmol) in DCM (1 mL) was treated with hydrogen chloride in 1,4-dioxane (842 mg, 5.77 mL, 4 M, 23.1 mmol). The mixture was stirred at room temperature for 30 minutes. The volatiles were removed under a stream of nitrogen, and the residue was further dried under vacuum. The resulting residue was taken up in DMF (6 mL) and treated with HATU (211 mg, 554 μmol) and DIPEA (299 mg, 0.40 mL, 2.31 mmol). The mixture was stirred at room temperature for 30 minutes. The reaction mixture was concentrated and purified by flash column chromatography (ethyl acetate/heptane) to afford 3⁵-(bis(4-methoxybenzyl)amino)-3³-chloro-2⁸-fluoro-2²-(methylthio)-9-aza-2(4,7)-pyrido[4,3-d]pyrimidina-1(1,3)-piperidina-3(1,2)-benzenacycloundecaphan-8-one (207 mg, 269 μmol, 58.2%).

MS (ESI): m/z (+)=769.5.

### Step E: Synthesis of 3⁵-amino-3³-chloro-2⁸-fluoro-2²-(((S)-1-methylpyrrolidin-2-yl)methoxy)-9-aza-2(4,7)-pyrido[4,3-d]pyrimidina-1(1,3)-piperidina-3(1,2)-benzenacycloundecaphan-8-one

A vial containing 3⁵-(bis(4-methoxybenzyl)amino)-3³-chloro-2⁸-fluoro-2²-(methylthio)-9-aza-2(4,7)-pyrido[4,3-d]pyrimidina-1(1,3)-piperidina-3(1,2)-benzenacycloundecaphan-8-one (27 mg, 35 μmol) under nitrogen was treated with a stock solution of mCPBA (17 mg, 75 wt %, 74 μmol) in anhydrous DMF (0.25 mL). The mixture was allowed to stir at room temperature for 4 hours. The reaction mixture was treated with (S)-(1-methylpyrrolidin-2-yl)methanol (4.0 mg, 35 μmol) and NaHMDS (29 mg, 0.16 mL, 1 M in THF, 0.16 mmol). The reaction mixture was stirred at room temperature for 2 hours. The reaction mixture was quenched with TFA (0.1 mL) and concentrated. The resulting residue was treated with TFA (0.27 mL, 3.5 mmol) and warmed at 60° C. for 1 hour. The reaction mixture was concentrated under a stream of nitrogen and was purified by HPLC (Method A) to afford 3⁵-amino-3³-chloro-2⁸-fluoro-2²-(((S)-1-methylpyrrolidin-2-yl)methoxy)-9-aza-2(4,7)-pyrido[4,3-d]pyrimidina-1(1,3)-piperidina-3(1,2)-benzenacycloundecaphan-8-one (1.0 mg, 1.7 μmol, 4.8%).

MS (ESI): m/z (+)=596.3.

¹H NMR (400 MHz, MeOD) δ 9.00 (s, 1H), 6.81 (d, J=2.4 Hz, 1H), 6.65 (d, J=2.4 Hz, 1H), 4.79-4.70 (m, 1H), 4.59-4.43 (m, 3H), 3.45-2.93 (m, 10H), 2.68-2.56 (m, 5H), 2.36-2.07 (m, 2H), 1.97-1.04 (m, 12H).

### Example 39. Synthesis of 3⁵-amino-3³-chloro-2⁸,3⁴-difluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacycloundecaphan-8-one (Compound 172a)

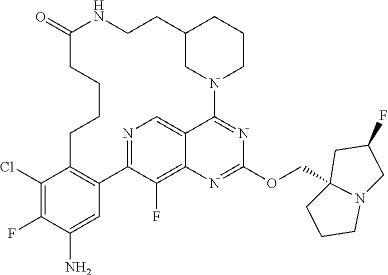 [SMILES: Nc1cc2c(c(Cl)c1F)CCCCC(=O)NCCC1CCCN(C1)c1nc(OC[C@@]34CCCN3C[C@H](F)C4)nc3c(F)c-2ncc13]

3⁵-amino-3³-chloro-2⁸,3⁴-difluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacycloundecaphan-8-one was prepared in an analogous manner to Example 29 by using methyl 5-(4-amino-2-chloro-3-fluoro-6-(4,4,5,5-tetramethyl-1,3,2-dioxaborolan-2-yl)phenyl)pentanoate in Step A.

MS (ESI): m/z (+)=658.4.

¹H NMR (400 MHz, MeOD) δ 8.99 (s, 1H), 6.81 (d, J=8.5 Hz, 1H), 5.40-5.20 (m, 1H), 4.78-4.67 (m, 1H), 4.59-4.51 (m, 1H), 4.39-4.22 (m, 2H), 3.48-3.11 (m, 8H), 3.11-2.92 (m, 2H), 2.69-2.56 (m, 1H), 2.42-1.58 (m, 12H), 1.58-1.10 (m, 6H).

### Example 40. Synthesis of 2⁸,3⁷-difluoro-2²-(((2R,7aS)-2-fluorotetrahydro-1H-pyrrolizin-7a(5H)-yl)methoxy)-3³-hydroxy-8-aza-1(4,6)-oxazepana-2(4,7)-pyrido[4,3-d]pyrimidina-3(1,8)-naphthalenacyclononaphan-7-one (Compound 173a)

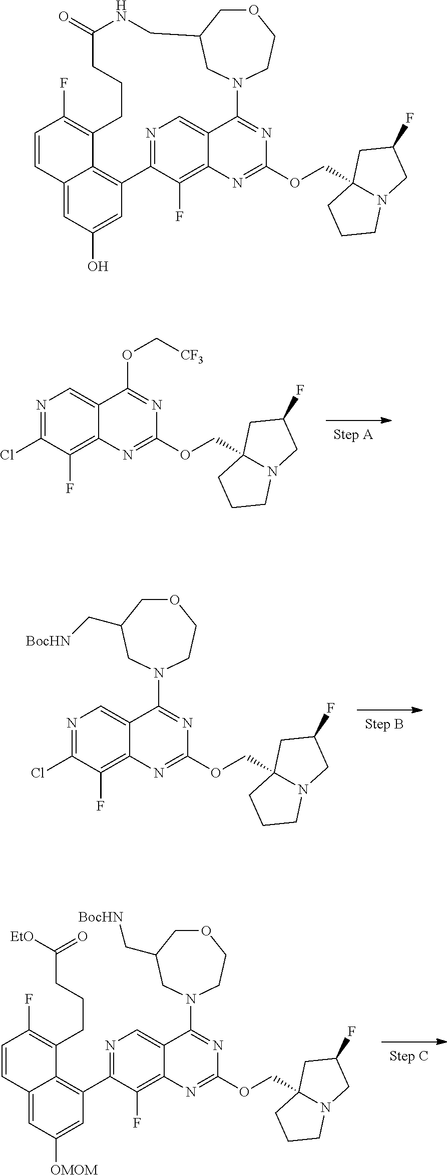

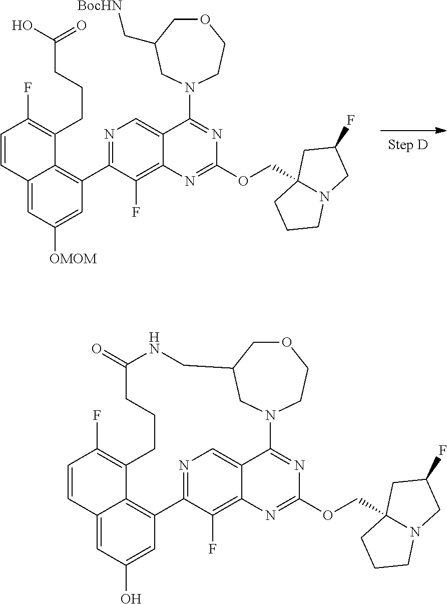

### Step A: Synthesis of tert-butyl ((4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)methyl)carbamate

A solution of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (700 mg, 1.60 mmol) and tert-butyl ((1,4-oxazepan-6-yl)methyl)carbamate (367 mg, 1.60 mmol) in DMSO (4 mL) was treated with DIPEA (1.03 g, 1.39 mL, 7.98 mmol) and warmed at 60° C. for 2 hours. The reaction mixture was purified by reverse-phase flash column chromatography using a gradient of 10-100% B (A: water+0.1% formic acid; B: acetonitrile+0.1% formic acid) to afford tert-butyl ((4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)methyl)carbamate (643 mg, 1.13 mmol, 70.8%).

MS (ESI): m/z (+)=569.2.

### Step B: Synthesis of ethyl 4-(8-(4-(6-(((tert-butoxycarbonyl)amino)methyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoate

A solution of tert-butyl ((4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)methyl)carbamate (180 mg, 316 μmol), ethyl 4-(2-fluoro-6-(methoxymethoxy)-8-(4,4,5,5-tetramethyl-1,3,2-dioxaborolan-2-yl)naphthalen-1-yl)butanoate (169 mg, 380 μmol), Cs₂CO₃ (309 mg, 949 μmol), and cataCXium A Pd G3 (23.0 mg, 31.6 μmol) in 1,4-dioxane (6 mL) and water (1 mL) was degassed with nitrogen. The mixture was warmed at 80° C. for 16 hours. The reaction mixture was concentrated and purified by reverse-phase flash column chromatography using a gradient of 10-100% B (A: water+0.1% formic acid; B: acetonitrile+0.1% formic acid) to afford ethyl 4-(8-(4-(6-(((tert-butoxycarbonyl)amino)methyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoate (154 mg, 181 μmol, 57.1%).

MS (ESI): m/z (+)=853.5.

### Step C: Synthesis of 4-(8-(4-(6-(((tert-butoxycarbonyl)amino)methyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoic acid

A solution of ethyl 4-(8-(4-(6-(((tert-butoxycarbonyl)amino)methyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoate (154 mg, 181 μmol) in THF (6 mL) and water (2 mL) was treated with LiOH·H₂O (75.8 mg, 1.81 mmol). The mixture was stirred at room temperature for 18 hours. The reaction mixture was concentrated under a stream of nitrogen and was purified by reverse-phase flash column chromatography using a gradient of 10-100% B (A: water+0.1% formic acid; B: acetonitrile+0.1% formic acid) to afford 4-(8-(4-(6-(((tert-butoxycarbonyl)amino)methyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoic acid (104 mg, 126 μmol, 69.8%).

MS (ESI): m/z (+)=825.5.

### Step D: Synthesis of 2⁸,3⁷-difluoro-2²-(((2R,7aS)-2-fluorotetrahydro-1H-pyrrolizin-7a(5H)-yl)methoxy)-3³-hydroxy-8-aza-1(4,6)-oxazepana-2(4,7)-pyrido[4,3-d]pyrimidina-3(1,8)-naphthalenacyclononaphan-7-one

A solution of 4-(8-(4-(6-(((tert-butoxycarbonyl)amino)methyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoic acid (104 mg, 126 μmol) in DCM (1 mL) was treated with HCl in 1,4-dioxane (230 mg, 1.58 mL, 4 M, 6.30 mmol). The mixture was stirred at room temperature for 30 minutes. The volatiles were removed under a stream of nitrogen and the residue was further dried under vacuum. The resulting residue was taken up in DMF (5 mL) and was treated with HATU (57.5 mg, 151 μmol) and DIPEA (81.5 mg, 0.11 mL, 630 μmol). The mixture was stirred at room temperature for 60 minutes. The reaction mixture was purified by HPLC (Method A) to afford 2⁸,3⁷-difluoro-2²-(((2R,7aS)-2-fluorotetrahydro-1H-pyrrolizin-7a(5H)-yl)methoxy)-3³-hydroxy-8-aza-1(4,6)-oxazepana-2(4,7)-pyrido[4,3-d]pyrimidina-3(1,8)-naphthalenacyclononaphan-7-one (30.8 mg, 46.5 μmol, 36.9%).

MS (ESI): m/z (+)=663.4.

¹H NMR (400 MHz, MeOD) δ 9.04 (s, 1H), 7.68-7.60 (m, 1H), 7.29-7.17 (m, 2H), 7.03-6.97 (m, 1H), 5.47-5.23 (m, 1H), 4.68-4.58 (m, 1H), 4.47-4.22 (m, 4H), 4.15-3.79 (m, 4H), 3.76-3.65 (m, 1H), 3.54-3.31 (m, 2H), 3.20-2.96 (m, 2H), 2.91-2.81 (m, 1H), 2.51-1.32 (m, 14H).

### Example 41. Synthesis of 3⁵-amino-3³-chloro-2⁸,3⁴-difluoro-2²-(((2R,7aS)-2-fluorotetrahydro-1H-pyrrolizin-7a(5H)-yl)methoxy)-9-aza-2(4,7)-quinazolina-1(1,3)-piperidina-3(1,2)-benzenacycloundecaphan-8-one (Compound 174a)

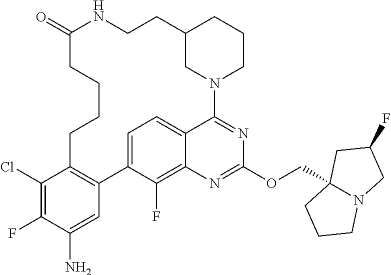 [SMILES: Nc1cc2c(c(Cl)c1F)CCCCC(=O)NCCC1CCCN(C1)c1nc(OC[C@@]34CCCN3C[C@H](F)C4)nc3c(F)c-2ccc13]

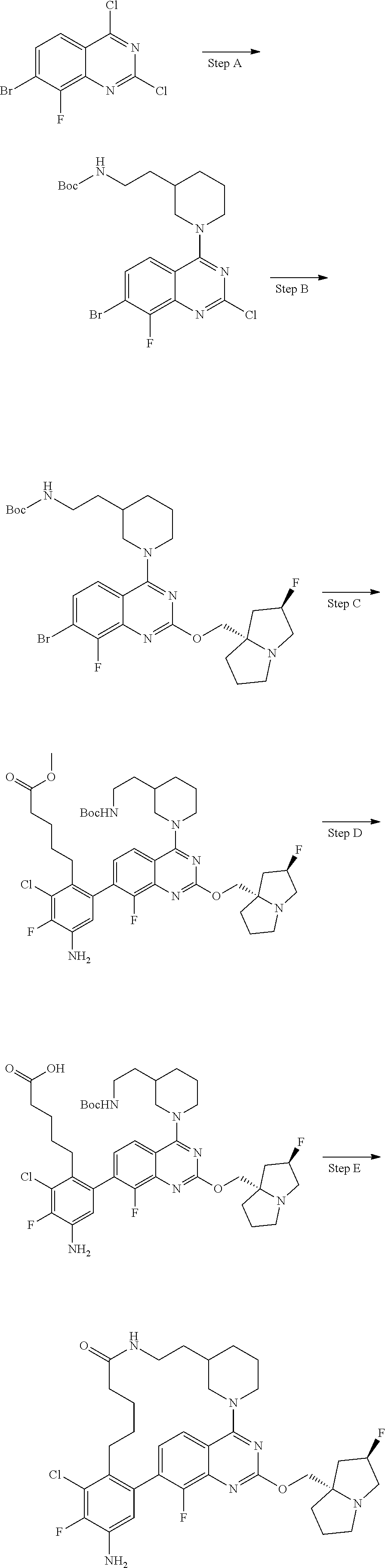 [SMILES: Fc1c(Br)ccc2c(Cl)nc(Cl)nc12 ; CC(C)(C)OC(=O)NCCC1CCCN(c2nc(Cl)nc3c(F)c(Br)ccc23)C1 ; CC(C)(C)OC(=O)NCCC1CCCN(c2nc(OC[C@@]34CCCN3C[C@H](F)C4)nc3c(F)c(Br)ccc23)C1 ; COC(=O)CCCCc1c(-c2ccc3c(N4CCCC(CCNC(=O)OC(C)(C)C)C4)nc(OC[C@@]45CCCN4C[C@H](F)C5)nc3c2F)cc(N)c(F)c1Cl ; CC(C)(C)OC(=O)NCCC1CCCN(c2nc(OC[C@@]34CCCN3C[C@H](F)C4)nc3c(F)c(-c4cc(N)c(F)c(Cl)c4CCCCC(=O)O)ccc23)C1 ; Nc1cc2c(c(Cl)c1F)CCCCC(=O)NCCC1CCCN(C1)c1nc(OC[C@@]34CCCN3C[C@H](F)C4)nc3c(F)c-2ccc13]

### Step A: Synthesis of tert-butyl (2-(1-(7-bromo-2-chloro-8-fluoroquinazolin-4-yl)piperidin-3-yl)ethyl)carbamate

A solution of 7-bromo-2,4-dichloro-8-fluoroquinazoline (500 mg, 1.69 mmol) in DCM (10 mL) under nitrogen was cooled to −78° C. Tert-butyl (2-(piperidin-3-yl)ethyl)carbamate (386 mg, 1.69 mmol) was added and the reaction was stirred for 5 minutes. DIPEA (1.09 g, 1.47 mL, 8.45 mmol) was added dropwise and the resulting reaction mixture was stirred at −78 C for 2 hours. The reaction mixture was allowed to slowly warm to room temperature, diluted with DCM, washed with water and brine, dried (Na₂SO₄), filtered, and concentrated to afford tert-butyl (2-(1-(7-bromo-2-chloro-8-fluoroquinazolin-4-yl)piperidin-3-yl)ethyl)carbamate (824 mg, 1.69 mmol, 100%) which was used directly in the next step without purification.

MS (ESI): m/z (+)=487.2.

### Step B: Synthesis of tert-butyl (2-(1-(7-bromo-8-fluoro-2-(((2R,7aS)-2-fluorotetrahydro-1H-pyrrolizin-7a(5H)-yl)methoxy)quinazolin-4-yl)piperidin-3-yl)ethyl)carbamate

A solution of ((2R,7aS)-2-fluorotetrahydro-1H-pyrrolizin-7a(5H)-yl)methanol (538 mg, 3.38 mmol), tert-butyl (2-(1-(7-bromo-2-chloro-8-fluoroquinazolin-4-yl)piperidin-3-yl)ethyl)carbamate (824 mg, 1.69 mmol), cataCXium A Pd G3 (185 mg, 253 μmol), and Cs₂CO₃ (1.65 g, 5.07 mmol) in 1,4-dioxane (10 mL) was degassed with nitrogen. The mixture was warmed at 70° C. for 2 hours. The reaction mixture was concentrated onto celite and was purified by reverse-phase flash column chromatography using a gradient of 10-100% B (A: water+0.1% formic acid; B: acetonitrile+0.1% formic acid) to afford tert-butyl (2-(1-(7-bromo-8-fluoro-2-(((2R,7aS)-2-fluorotetrahydro-1H-pyrrolizin-7a(5H)-yl)methoxy)quinazolin-4-yl)piperidin-3-yl)ethyl)carbamate (786 mg, 1.29 mmol, 76.2%).

MS (ESI): m/z (+)=610.2.

### Step C: Synthesis of methyl 5-(4-amino-6-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)quinazolin-7-yl)-2-chloro-3-fluorophenyl)pentanoate

A solution of tert-butyl (2-(1-(7-bromo-8-fluoro-2-(((2R,7aS)-2-fluorotetrahydro-1H-pyrrolizin-7a(5H)-yl)methoxy)quinazolin-4-yl)piperidin-3-yl)ethyl)carbamate (160 mg, 262 μmol), methyl 5-(4-amino-2-chloro-3-fluoro-6-(4,4,5,5-tetramethyl-1,3,2-dioxaborolan-2-yl)phenyl)pentanoate (101 mg, 262 μmol), Cs₂CO₃ (256 mg, 786 μmol), and cataCXium A Pd G3 (19.1 mg, 26.2 μmol) in 1,4-dioxane (4 mL) and water (0.5 mL) was warmed at 80° C. for 16 hours. The reaction mixture was concentrated onto celite and purified by reverse-phase flash column chromatography using a gradient of 10-100% B (A: water+0.1% formic acid; B: acetonitrile+0.1% formic acid) to afford methyl 5-(4-amino-6-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)quinazolin-7-yl)-2-chloro-3-fluorophenyl)pentanoate (27 mg, 34 μmol, 13%).

MS (ESI): m/z (+)=789.5.

### Step D: Synthesis of 5-(4-amino-6-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)quinazolin-7-yl)-2-chloro-3-fluorophenyl)pentanoic acid

A solution of methyl 5-(4-amino-6-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)quinazolin-7-yl)-2-chloro-3-fluorophenyl)pentanoate (27 mg, 34 μmol) in THF (2 mL), water (0.5 mL), and MeOH (0.5 mL) was treated with LiOH·H₂O (14 mg, 0.34 mmol). The mixture was stirred at room temperature for 16 hours. The reaction mixture was concentrated and purified by reverse-phase flash column chromatography using a gradient of 10-100% B (A: water+0.1% formic acid; B: acetonitrile+0.1% formic acid) to afford 5-(4-amino-6-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)quinazolin-7-yl)-2-chloro-3-fluorophenyl)pentanoic acid (6.6 mg, 8.5 μmol, 25%).

MS (ESI): m/z (+)=775.5.

### Step E: Synthesis of 3⁵-amino-3³-chloro-2⁸,3⁴-difluoro-2²-(((2R,7aS)-2-fluorotetrahydro-1H-pyrrolizin-7a(5H)-yl)methoxy)-9-aza-2(4,7)-quinazolina-1(1,3)-piperidina-3(1,2)-benzenacycloundecaphan-8-one

A solution of 5-(4-amino-6-(4-(3-(2-((tert-butoxycarbonyl)amino)ethyl)piperidin-1-yl)-8-fluoro-2-(((2R,7aS)-2-fluorotetrahydro-1H-pyrrolizin-7a(5H)-yl)methoxy)quinazolin-7-yl)-2-chloro-3-fluorophenyl)pentanoic acid (6.6 mg, 8.5 μmol) in DCM (1 mL) was treated with HCl in 1,4-dioxane (0.11 mL, 4 M, 0.43 mmol). The mixture was stirred at room temperature for 30 minutes. The volatiles were removed under a stream of nitrogen and the residue was further dried under vacuum. The resulting residue was taken up in DMF (1 mL) and was treated with HATU (3.9 mg, 10 μmol) and DIPEA (5.5 mg, 7.4 μL, 43 μmol). The mixture was stirred at room temperature for 30 minutes. The reaction mixture was purified by HPLC (Method A) to afford 3⁵-amino-3³-chloro-2⁸,3⁴-difluoro-2²-(((2R,7aS)-2-fluorotetrahydro-1H-pyrrolizin-7a(5H)-yl)methoxy)-9-aza-2(4,7)-quinazolina-1(1,3)-piperidina-3(1,2)-benzenacycloundecaphan-8-one (4.9 mg, 7.5 μmol, 88%).

MS (ESI): m/z (+)=657.4.

¹H NMR (400 MHz, MeOD) δ 7.79-7.69 (m, 1H), 7.15-7.07 (m, 1H), 6.68-6.61 (m, 1H), 5.49-5.26 (m, 1H), 4.61-4.30 (m, 3H), 3.72-3.28 (m, 4H), 3.20-2.94 (m, 2H), 2.89-0.97 (m, 24H).

### Example 42. Synthesis of 2⁸,3⁷-difluoro-2²-(((2R,7aS)-2-fluorotetrahydro-1H-pyrrolizin-7a(5H)-yl)methoxy)-3³-hydroxy-8-aza-2(4,7)-quinazolina-1(1,3)-piperidina-3(1,8)-naphthalenacyclodecaphan-7-one (Compound 175a)

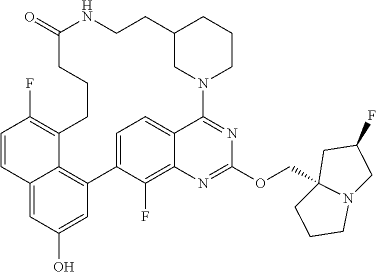 [SMILES: O=C1CCCc2c(F)ccc3cc(O)cc(c23)-c2ccc3c(nc(OC[C@@]45CCCN4C[C@H](F)C5)nc3c2F)N2CCCC(CCN1)C2]

2⁸,3⁷-difluoro-2²-(((2R,7aS)-2-fluorotetrahydro-1H-pyrrolizin-7a(5H)-yl)methoxy)-3³-hydroxy-8-aza-2(4,7)-quinazolina-1(1,3)-piperidina-3(1,8)-naphthalenacyclodecaphan-7-one was prepared in an analogous manner to Example 41 by using ethyl 4-(2-fluoro-6-(methoxymethoxy)-8-(4,4,5,5-tetramethyl-1,3,2-dioxaborolan-2-yl)naphthalen-1-yl)butanoate in Step C.

MS (ESI): m/z (+)=660.4.

¹H NMR (400 MHz, MeOD) δ 7.78-7.69 (m, 1H), 7.70-7.55 (m, 1H), 7.30-7.11 (m, 3H), 6.90-6.82 (m, 1H), 5.49-5.24 (m, 1H), 4.78-4.68 (m, 1H), 4.50-4.23 (m, 2H), 3.68-2.80 (m, 11H), 2.80-2.64 (m, 1H), 2.52-0.93 (m, 16H).

### Example 43. Synthesis of 3⁵-amino-3³-chloro-2⁸-fluoro-2²-(2-((S)-1-methylpyrrolidin-2-yl)ethoxy)-9-aza-2(4,7)-pyrido[4,3-d]pyrimidina-1(1,3)-piperidina-3(1,2)-benzenacycloundecaphan-8-one (Compound 176a)

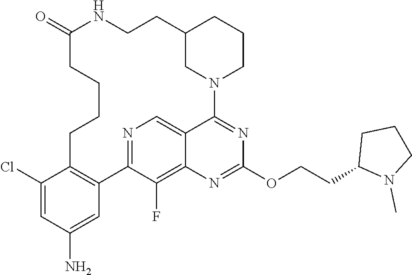 [SMILES: CN1CCC[C@H]1CCOc1nc2c3cnc(c(F)c3n1)-c1cc(N)cc(Cl)c1CCCCC(=O)NCCC1CCCN2C1]

3⁵-amino-3³-chloro-2⁸-fluoro-2²-(2-((S)-1-methylpyrrolidin-2-yl)ethoxy)-9-aza-2(4,7)-pyrido[4,3-d]pyrimidina-1(1,3)-piperidina-3(1,2)-benzenacycloundecaphan-8-one was prepared in an analogous manner to Example 38 by using (S)-2-(1-methylpyrrolidin-2-yl)ethan-1-ol in Step E.

MS (ESI): m/z (+)=610.4.

¹H NMR (400 MHz, MeOD) δ 8.97 (s, 1H), 6.80 (d, J=2.4 Hz, 1H), 6.65 (d, J=2.4 Hz, 1H), 4.76-4.68 (m, 1H), 4.58-4.37 (m, 3H), 3.43-2.92 (m, 6H), 2.66-2.55 (m, 1H), 2.49-2.19 (m, 7H), 2.15-2.06 (m, 1H), 1.92-1.08 (m, 18H).

### Example 44. Synthesis of 3⁵-amino-3³-chloro-2⁸-fluoro-2²-((1-(morpholinomethyl)cyclopropyl)methoxy)-9-aza-2(4,7)-pyrido[4,3-d]pyrimidina-1(1,3)-piperidina-3(1,2)-benzenacycloundecaphan-8-one (Compound 177)

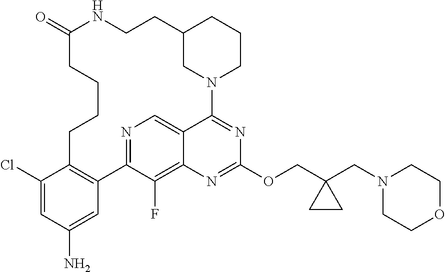 [SMILES: Nc1cc(Cl)c2c(c1)-c1ncc3c(nc(OCC4(CN5CCOCC5)CC4)nc3c1F)N1CCCC(CCNC(=O)CCCC2)C1]

3⁵-amino-3³-chloro-2⁸-fluoro-2²-((1-(morpholinomethyl)cyclopropyl)methoxy)-9-aza-2(4,7)-pyrido[4,3-d]pyrimidina-1(1,3)-piperidina-3(1,2)-benzenacycloundecaphan-8-one was prepared in an analogous manner to Example 38 by using (1-(morpholinomethyl)cyclopropyl)methanol in Step E.

LCMS: Retention time=2.06 min, m/z (+)=652.4.

### Example 45. Synthesis of 3⁵-amino-3³-chloro-2²-((1-((dimethylamino)methyl)cyclopropyl)methoxy)-2⁸-fluoro-9-aza-2(4,7)-pyrido[4,3-d]pyrimidina-1(1,3)-piperidina-3(1,2)-benzenacycloundecaphan-8-one (Compound 178)

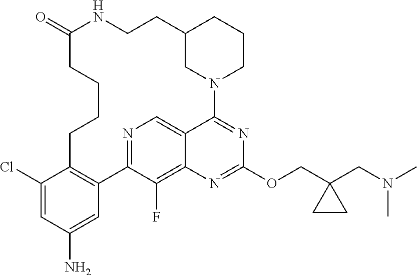 [SMILES: CN(C)CC1(COc2nc3c4cnc(c(F)c4n2)-c2cc(N)cc(Cl)c2CCCCC(=O)NCCC2CCCN3C2)CC1]

3⁵-amino-3³-chloro-2²-((1-((dimethylamino)methyl)cyclopropyl)methoxy)-2⁸-fluoro-9-aza-2(4,7)-pyrido[4,3-d]pyrimidina-1(1,3)-piperidina-3(1,2)-benzenacycloundecaphan-8-one was prepared in an analogous manner to Example 38 by using (1-((dimethylamino)methyl)cyclopropyl)methanol in Step E.

MS (ESI): m/z (+)=610.4.

¹H NMR (400 MHz, MeOD) δ 8.97 (s, 1H), 6.81 (d, J=2.4 Hz, 1H), 6.65 (d, J=2.4 Hz, 1H), 4.75-4.67 (m, 1H), 4.58-4.50 (m, 1H), 4.38-4.26 (m, 2H), 3.43-2.93 (m, 6H), 2.66-2.51 (m, 3H), 2.45-2.25 (m, 8H), 1.92-1.30 (m, 8H), 1.26-1.08 (m, 2H), 0.74-0.69 (m, 2H), 0.60-0.51 (m, 2H).

### Example 46. Synthesis of 3⁵-amino-3³-chloro-2⁸-fluoro-2²-(((2S,4R)-4-fluoro-1-methylpyrrolidin-2-yl)methoxy)-9-aza-2(4,7)-pyrido[4,3-d]pyrimidina-1(1,3)-piperidina-3(1,2)-benzenacycloundecaphan-8-one (Compound 179a)

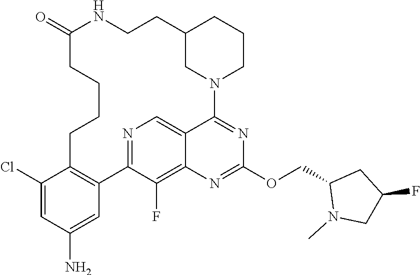 [SMILES: CN1C[C@H](F)C[C@H]1COc1nc2c3cnc(c(F)c3n1)-c1cc(N)cc(Cl)c1CCCCC(=O)NCCC1CCCN2C1]

3⁵-amino-3³-chloro-2⁸-fluoro-2²-(((2S,4R)-4-fluoro-1-methylpyrrolidin-2-yl)methoxy)-9-aza-2(4,7)-pyrido[4,3-d]pyrimidina-1(1,3)-piperidina-3(1,2)-benzenacycloundecaphan-8-one was prepared in an analogous manner to Example 38 by using ((2S,4R)-4-fluoro-1-methylpyrrolidin-2-yl)methanol in Step E.

MS (ESI): m/z (+)=614.4.

¹H NMR (400 MHz, MeOD) δ 8.98 (s, 1H), 6.80 (d, J=2.4 Hz, 1H), 6.65 (d, J=2.4 Hz, 1H), 5.22-5.03 (m, 1H), 4.75-4.67 (m, 1H), 4.57-4.40 (m, 3H), 3.52-3.06 (m, 6H), 3.02-2.93 (m, 1H), 2.68-2.55 (m, 2H), 2.51 (s, 3H), 2.36-2.16 (m, 2H), 2.03-1.58 (m, 7H), 1.58-1.33 (m, 5H), 1.27-1.08 (m, 2H).

### Example 47. Synthesis of 3⁵-amino-3³-chloro-2⁸,3⁴-difluoro-2²-(((2R,7aS)-2-fluorotetrahydro-1H-pyrrolizin-7a(5H)-yl)methoxy)-13-hydroxy-9-aza-2(4,7)-quinazolina-1(1,3)-piperidina-3(1,2)-benzenacycloundecaphan-8-one (Compound 180a)

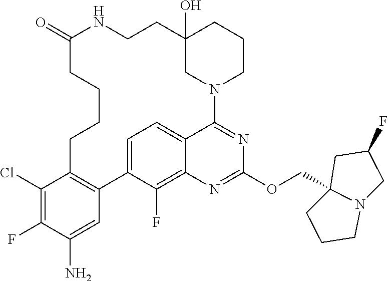 [SMILES: Nc1cc2c(c(Cl)c1F)CCCCC(=O)NCCC1(O)CCCN(C1)c1nc(OC[C@@]34CCCN3C[C@H](F)C4)nc3c(F)c-2ccc13]

3⁵-amino-3³-chloro-2⁸,3⁴-difluoro-2²-(((2R,7aS)-2-fluorotetrahydro-1H-pyrrolizin-7a(5H)-yl)methoxy)-13-hydroxy-9-aza-2(4,7)-quinazolina-1(1,3)-piperidina-3(1,2)-benzenacycloundecaphan-8-one was prepared in an analogous manner to Example 41 by using tert-butyl (2-(3-hydroxypiperidin-3-yl)ethyl)carbamate in Step A. MS (ESI): m/z (+)=673.4.

¹H NMR (400 MHz, MeOD) δ 7.81-7.66 (m, 1H), 7.16-7.05 (m, 1H), 6.64 (d, J=8.4 Hz, 1H), 5.51-5.27 (m, 1H), 4.62-4.49 (m, 1H), 4.49-4.30 (m, 3H), 3.72-3.14 (m, 6H), 3.09-2.96 (m, 1H), 2.88-2.68 (m, 2H), 2.56-0.96 (m, 19H).

### Example 48. Synthesis of 3⁵-amino-2⁸-fluoro-2²-(((2R,7aS)-2-fluorotetrahydro-1H-pyrrolizin-7a(5H)-yl)methoxy)-3³-(trifluoromethyl)-9-aza-2(4,7)-pyrido[4,3-d]pyrimidina-1(1,3)-piperidina-3(1,2)-benzenacycloundecaphan-8-one (Compound 181a)

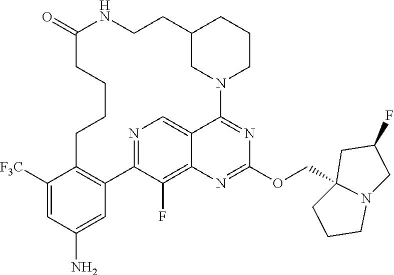 [SMILES: Nc1cc2c(c(C(F)(F)F)c1)CCCCC(=O)NCCC1CCCN(C1)c1nc(OC[C@@]34CCCN3C[C@H](F)C4)nc3c(F)c-2ncc13]

3⁵-amino-2⁸-fluoro-2²-(((2R,7aS)-2-fluorotetrahydro-1H-pyrrolizin-7a(5H)-yl)methoxy)-3³-(trifluoromethyl)-9-aza-2(4,7)-pyrido[4,3-d]pyrimidina-1(1,3)-piperidina-3(1,2)-benzenacycloundecaphan-8-one was prepared in an analogous manner to Example 29 by using methyl 5-(4-amino-2-(4,4,5,5-tetramethyl-1,3,2-dioxaborolan-2-yl)-6-(trifluoromethyl)phenyl)pentanoate in Step A.

MS (ESI): m/z (+)=674.4.

¹H NMR (400 MHz, MeOD) δ 9.01 (s, 1H), 7.02 (d, J=2.5 Hz, 1H), 6.83 (d, J=2.4 Hz, 1H), 5.43-5.20 (m, 1H), 4.75-4.62 (m, 1H), 4.61-4.53 (m, 1H), 4.46-4.27 (m, 2H), 3.59-3.34 (m, 4H), 3.20-3.07 (m, 2H), 3.05-2.92 (m, 1H), 2.61-0.93 (m, 22H).

### Example 49. Synthesis of 3⁵-amino-3³-chloro-2⁸,3⁴-difluoro-2²-(((2R,7aS)-2-fluorotetrahydro-1H-pyrrolizin-7a(5H)-yl)methoxy)-9-aza-1(4,6)-oxazepana-2(4,7)-pyrido[4,3-d]pyrimidina-3(1,2)-benzenacyclodecaphan-8-one (Compound 182a)

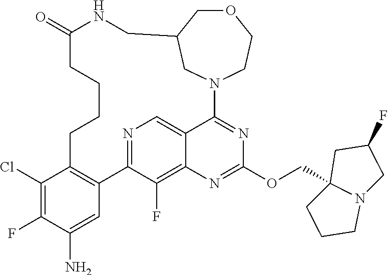 [SMILES: Nc1cc2c(c(Cl)c1F)CCCCC(=O)NCC1COCCN(C1)c1nc(OC[C@@]34CCCN3C[C@H](F)C4)nc3c(F)c-2ncc13]

3⁵-amino-3³-chloro-2⁸,3⁴-difluoro-2²-(((2R,7aS)-2-fluorotetrahydro-1H-pyrrolizin-7a(5H)-yl)methoxy)-9-aza-1(4,6)-oxazepana-2(4,7)-pyrido[4,3-d]pyrimidina-3(1,2)-benzenacyclodecaphan-8-one was prepared in an analogous manner to Example 29 by using tert-butyl ((4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)methyl)carbamate and methyl 5-(4-amino-2-chloro-3-fluoro-6-(4,4,5,5-tetramethyl-1,3,2-dioxaborolan-2-yl)phenyl)pentanoate in Step A.

MS (ESI): m/z (+)=660.4.

¹H NMR (400 MHz, MeOD) δ 8.98 (s, 1H), 6.84 (d, J=8.5 Hz, 1H), 5.43-5.23 (m, 1H), 4.41-4.27 (m, 2H), 4.23-4.12 (m, 2H), 4.09-3.81 (m, 5H), 3.52-3.32 (m, 4H), 3.17-2.98 (m, 2H), 2.58-1.77 (m, 12H), 1.50-1.28 (m, 2H), 1.19-1.04 (m, 2H).

### Example 50. Synthesis of 3⁵-amino-3³-chloro-2⁶,2⁸,3⁴-trifluoro-2²-(((2R,7aS)-2-fluorotetrahydro-1H-pyrrolizin-7a(5H)-yl)methoxy)-1³-hydroxy-9-aza-2(4,7)-quinazolina-1(1,3)-piperidina-3(1,2)-benzenacycloundecaphan-8-one (Compound 183a)

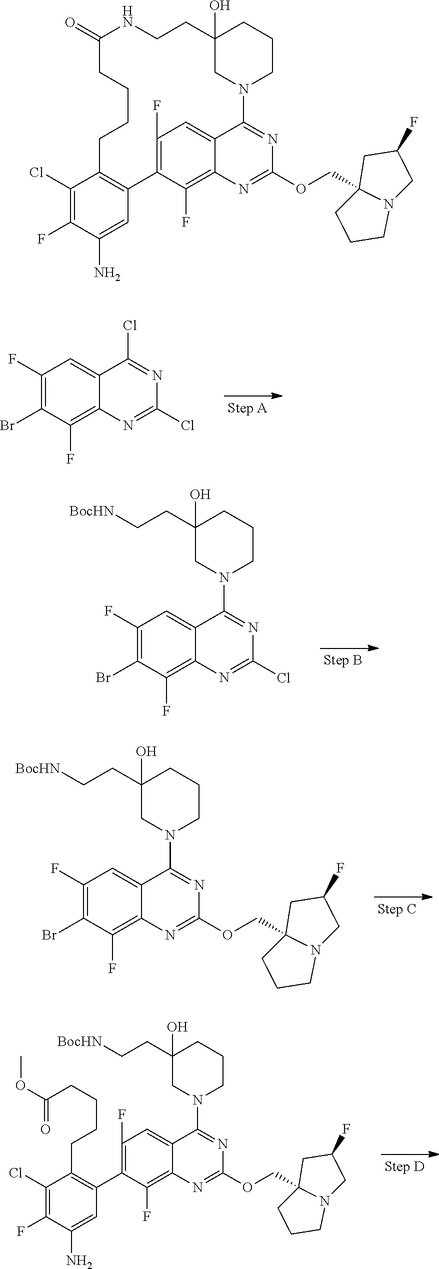 [SMILES: Nc1cc2c(c(Cl)c1F)CCCCC(=O)NCCC1(O)CCCN(C1)c1nc(OC[C@@]34CCCN3C[C@H](F)C4)nc3c(F)c-2c(F)cc13 ; Fc1cc2c(Cl)nc(Cl)nc2c(F)c1Br ; CC(C)(C)OC(=O)NCCC1(O)CCCN(c2nc(Cl)nc3c(F)c(Br)c(F)cc23)C1 ; CC(C)(C)OC(=O)NCCC1(O)CCCN(c2nc(OC[C@@]34CCCN3C[C@H](F)C4)nc3c(F)c(Br)c(F)cc23)C1 ; COC(=O)CCCCc1c(-c2c(F)cc3c(N4CCCC(O)(CCNC(=O)OC(C)(C)C)C4)nc(OC[C@@]45CCCN4C[C@H](F)C5)nc3c2F)cc(N)c(F)c1Cl]

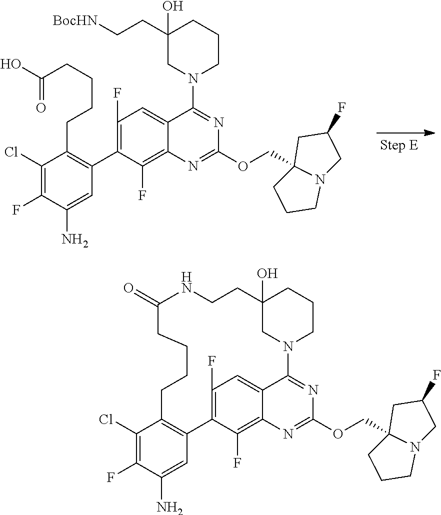 [SMILES: CC(C)(C)OC(=O)NCCC1(O)CCCN(c2nc(OC[C@@]34CCCN3C[C@H](F)C4)nc3c(F)c(-c4cc(N)c(F)c(Cl)c4CCCCC(=O)O)c(F)cc23)C1 ; Nc1cc2c(c(Cl)c1F)CCCCC(=O)NCCC1(O)CCCN(C1)c1nc(OC[C@@]34CCCN3C[C@H](F)C4)nc3c(F)c-2c(F)cc13]

### Step A: Synthesis of tert-butyl (2-(1-(7-bromo-2-chloro-6,8-difluoroquinazolin-4-yl)-3-hydroxypiperidin-3-yl)ethyl)carbamate

A solution of 7-bromo-2,4-dichloro-6,8-difluoroquinazoline (333 mg, 1.06 mmol) in DCM (5 mL) under nitrogen was cooled to −78° C. Tert-butyl (2-(3-hydroxypiperidin-3-yl)ethyl)carbamate (259 mg, 1.06 mmol) was added and the reaction was stirred for 5 minutes. DIPEA (686 mg, 924 μL, 5.30 mmol) was added dropwise. The resulting reaction mixture was stirred at −78° C. for 10 minutes and then allowed to warm to room temperature. The mixture was stirred for 3 hours. The reaction mixture was purified directly by flash column chromatography (ethyl acetate/heptane) to afford tert-butyl (2-(1-(7-bromo-2-chloro-6,8-difluoroquinazolin-4-yl)-3-hydroxypiperidin-3-yl)ethyl)carbamate (561 mg, 1.0 mmol, 96%).

MS (ESI): m/z (+)=521.2.

### Step B: Synthesis of tert-butyl (2-(1-(7-bromo-6,8-difluoro-2-(((2R,7aS)-2-fluorotetrahydro-1H-pyrrolizin-7a(5H)-yl)methoxy)quinazolin-4-yl)-3-hydroxypiperidin-3-yl)ethyl)carbamate

A solution of ((2R,7aS)-2-fluorotetrahydro-1H-pyrrolizin-7a(5H)-yl)methanol (342 mg, 2.15 mmol), tert-butyl (2-(1-(7-bromo-2-chloro-6,8-difluoroquinazolin-4-yl)-3-hydroxypiperidin-3-yl)ethyl)carbamate (561 mg, 1.08 mmol), cataCXium A Pd G3 (117 mg, 161 μmol), and Cs₂CO₃ (1.05 g, 3.23 mmol) in 1,4-dioxane (10 mL) was degassed with nitrogen and was warmed at 70° C. for 2 hours. The reaction mixture was concentrated onto celite and was purified by reverse-phase flash column chromatography using a gradient of 10-100% B (A: water+0.1% formic acid; B: acetonitrile+0.1% formic acid) to afford tert-butyl (2-(1-(7-bromo-6,8-difluoro-2-(((2R,7aS)-2-fluorotetrahydro-1H-pyrrolizin-7a(5H)-yl)methoxy)quinazolin-4-yl)-3-hydroxypiperidin-3-yl)ethyl)carbamate (569 mg, 883 μmol, 82.1%).

MS (ESI): m/z (+)=644.3.

### Step C: Synthesis of methyl 5-(4-amino-6-(4-(3-(2-((tert-butoxycarbonyl)amino)ethyl)-3-hydroxypiperidin-1-yl)-6,8-difluoro-2-(((2R,7aS)-2-fluorotetrahydro-1H-pyrrolizin-7a(5H)-yl)methoxy)quinazolin-7-yl)-2-chloro-3-fluorophenyl)pentanoate

A solution of tert-butyl (2-(1-(7-bromo-6,8-difluoro-2-(((2R,7aS)-2-fluorotetrahydro-1H-pyrrolizin-7a(5H)-yl)methoxy)quinazolin-4-yl)-3-hydroxypiperidin-3-yl)ethyl)carbamate (150 mg, 233 μmol), methyl 5-(4-amino-2-chloro-3-fluoro-6-(4,4,5,5-tetramethyl-1,3,2-dioxaborolan-2-yl)phenyl)pentanoate (98.7 mg, 256 μmol), Cs₂CO₃ (227 mg, 698 μmol), and cataCXium A Pd G3 (16.9 mg, 23.3 μmol) in 1,4-dioxane (4 mL) and water (0.5 mL) was warmed at 80° C. for 2 hours. The reaction mixture was concentrated onto celite and was purified by reverse-phase flash column chromatography using a gradient of 10-100% B (A: water+0.1% formic acid; B: acetonitrile+0.1% formic acid) to afford methyl 5-(4-amino-6-(4-(3-(2-((tert-butoxycarbonyl)amino)ethyl)-3-hydroxypiperidin-1-yl)-6,8-difluoro-2-(((2R,7aS)-2-fluorotetrahydro-1H-pyrrolizin-7a(5H)-yl)methoxy)quinazolin-7-yl)-2-chloro-3-fluorophenyl)pentanoate (88 mg, 0.11 mmol, 46%).

MS (ESI): m/z (+)=823.4.

### Step D: Synthesis of 5-(4-amino-6-(4-(3-(2-((tert-butoxycarbonyl)amino)ethyl)-3-hydroxypiperidin-1-yl)-6,8-difluoro-2-(((2R,7aS)-2-fluorotetrahydro-1H-pyrrolizin-7a(5H)-yl)methoxy)quinazolin-7-yl)-2-chloro-3-fluorophenyl)pentanoic acid

A solution of methyl 5-(4-amino-6-(4-(3-(2-((tert-butoxycarbonyl)amino)ethyl)-3-hydroxypiperidin-1-yl)-6,8-difluoro-2-(((2R,7aS)-2-fluorotetrahydro-1H-pyrrolizin-7a(5H)-yl)methoxy)quinazolin-7-yl)-2-chloro-3-fluorophenyl)pentanoate (88 mg, 0.11 mmol) in THE (4 mL) and water (1 mL)/MeOH (1 mL) was treated with LiOH·H₂O (45 mg, 1.1 mmol). The mixture was stirred at room temperature for 3 hours. The reaction mixture was concentrated and was purified by reverse-phase flash column chromatography using a gradient of 10-100% B (A: water+0.1% formic acid; B: acetonitrile+0.1% formic acid) to afford 5-(4-amino-6-(4-(3-(2-((tert-butoxycarbonyl)amino)ethyl)-3-hydroxypiperidin-1-yl)-6,8-difluoro-2-(((2R,7aS)-2-fluorotetrahydro-1H-pyrrolizin-7a(5H)-yl)methoxy)quinazolin-7-yl)-2-chloro-3-fluorophenyl)pentanoic acid (69 mg, 85 μmol, 80%).

MS (ESI): m/z (+)=809.4.

### Step E: Synthesis of 3⁵-amino-3³-chloro-2⁶,2⁸,3⁴-trifluoro-2²-(((2R,7aS)-2-fluorotetrahydro-1H-pyrrolizin-7a(5H)-yl)methoxy)-1³-hydroxy-9-aza-2(4,7)-quinazolina-1(1,3)-piperidina-3(1,2)-benzenacycloundecaphan-8-one

A solution of 5-(4-amino-6-(4-(3-(2-((tert-butoxycarbonyl)amino)ethyl)-3-hydroxypiperidin-1-yl)-6,8-difluoro-2-(((2R,7aS)-2-fluorotetrahydro-1H-pyrrolizin-7a(5H)-yl)methoxy)quinazolin-7-yl)-2-chloro-3-fluorophenyl)pentanoic acid (69 mg, 85 μmol) in DCM (1 mL) was treated with hydrogen chloride (0.16 g, 1.1 mL, 4 M, 4.3 mmol). The mixture was stirred at room temperature for 30 minutes. The volatiles were removed under a stream of nitrogen and the residue was further dried under vacuum. The resulting residue was taken up in DMF (5 mL) and was treated with HATU (39 mg, 0.10 mmol) and DIPEA (55 mg, 74 μL, 0.43 mmol). The mixture was stirred at room temperature for 30 minutes. The reaction mixture was purified by HPLC (Method A) to afford 3⁵-amino-3³-chloro-2⁶,2⁸,3⁴-trifluoro-2²-(((2R,7aS)-2-fluorotetrahydro-1H-pyrrolizin-7a(5H)-yl)methoxy)-1³-hydroxy-9-aza-2(4,7)-quinazolina-1(1,3)-piperidina-3(1,2)-benzenacycloundecaphan-8-one (37.0 mg, 53.5 μmol, 63%).

MS (ESI): m/z (+)=691.3.

### Example 51. Synthesis of 3⁵-amino-3³-chloro-2⁶,2⁸,3⁴-trifluoro-2²-(((2R,7aS)-2-fluorotetrahydro-1H-pyrrolizin-7a(5H)-yl)methoxy)-9-aza-1(4,6)-oxazepana-2(4,7)-quinazolina-3(1,2)-benzenacyclodecaphan-8-one (Compound 184a)

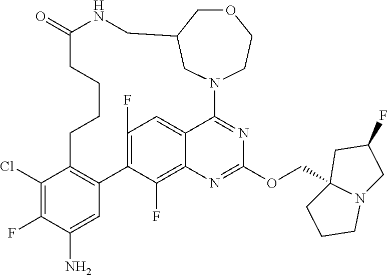 [SMILES: Nc1cc2c(c(Cl)c1F)CCCCC(=O)NCC1COCCN(C1)c1nc(OC[C@@]34CCCN3C[C@H](F)C4)nc3c(F)c-2c(F)cc13]

3⁵-amino-3³-chloro-2⁶,2⁸,3⁴-trifluoro-2²-(((2R,7aS)-2-fluorotetrahydro-1H-pyrrolizin-7a(5H)-yl)methoxy)-9-aza-1(4,6)-oxazepana-2(4,7)-quinazolina-3(1,2)-benzenacyclodecaphan-8-one was prepared in an analogous manner to Example 50 by using tert-butyl ((1,4-oxazepan-6-yl)methyl)carbamate in Step A.

LCMS: Retention time=2.11 min, m/z (+)=677.4.

### Example 52. Synthesis of 3⁵-amino-3³-chloro-2⁸,3⁴-difluoro-2²-(((2R,7aS)-2-fluorotetrahydro-1H-pyrrolizin-7a(5H)-yl)methoxy)-9-aza-1(4,6)-oxazepana-2(4,7)-pyrido[4,3-d]pyrimidina-3(1,2)-benzenacycloundecaphan-8-one (Compound 185a)

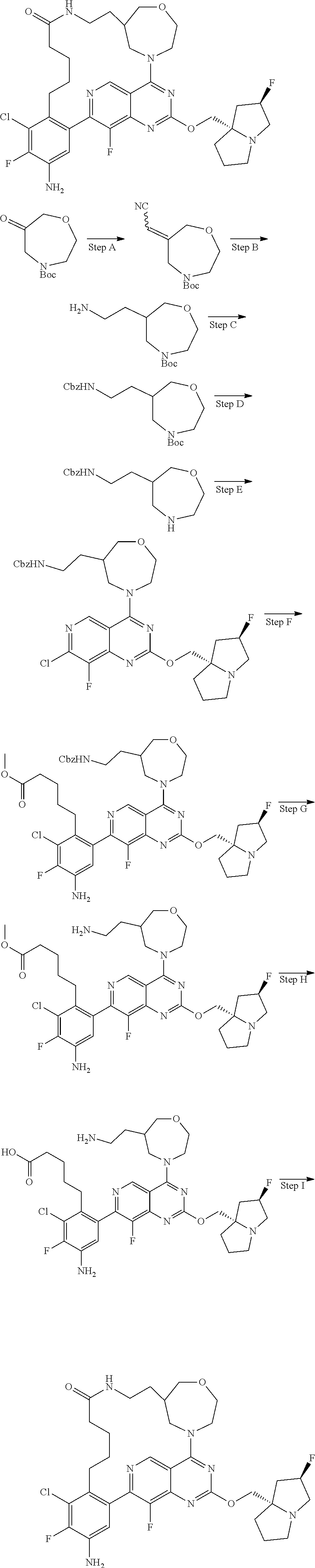

### Step A: Synthesis of tert-butyl 6-(cyanomethylene)-1,4-oxazepane-4-carboxylate

A solution of NaH (1.07 g, 60 wt %, 26.8 mmol) in THF (40 mL) at 0° C. under nitrogen was treated dropwise with diethyl cyanomethylphosphonate (4.74 g, 4.33 mL, 26.8 mmol). The mixture was stirred at room temperature for 15 minutes. Tert-butyl 6-oxo-1,4-oxazepane-4-carboxylate (4800 mg, 22.3 mmol) was added, and the resulting reaction mixture was stirred at room temperature for 4 hours. The reaction mixture was quenched with the addition of saturated aqueous NaHCO₃. The mixture was extracted with EtOAc, dried (Na₂SO₄), filtered, and concentrated. The resulting residue was purified by flash column chromatography (0-80% ethyl acetate/heptane) to afford tert-butyl 6-(cyanomethylene)-1,4-oxazepane-4-carboxylate (5.01 g, 21.0 mmol, 94.3%).

¹H NMR (400 MHz, CDCl₃) δ 5.43-5.19 (m, 1H), 4.59-4.35 (m, 2H), 4.30-4.07 (m, 2H), 3.85-3.67 (m, 2H), 3.63-3.45 (m, 2H), 1.52-1.38 (m, 9H).

### Step B: Synthesis of tert-butyl 6-(2-aminoethyl)-1,4-oxazepane-4-carboxylate

A solution of tert-butyl 6-(cyanomethylene)-1,4-oxazepane-4-carboxylate (520 mg, 2.18 mmol) and ammonium hydroxide (306 mg, 348 μL, 8.73 mmol) in MeOH (10 mL) at 0° C. was treated with Raney Nickel (64.0 mg, 8.71 μL, 1.09 mmol). The mixture was stirred at room temperature for 3 hours. The reaction mixture was filtered through celite and concentrated to afford tert-butyl 6-(2-aminoethyl)-1,4-oxazepane-4-carboxylate (533 mg, 2.18 mmol, 100%), which was used directly in the next step without purification.

MS (ESI): m/z (+)=245.2.

### Step C: Synthesis of tert-butyl 6-(2-(((benzyloxy)carbonyl)amino)ethyl)-1,4-oxazepane-4-carboxylate

A solution of tert-butyl 6-(2-aminoethyl)-1,4-oxazepane-4-carboxylate (4.8 g, 20 mmol) and Et₃N (6.0 g, 8.2 mL, 59 mmol) in THF (60 mL) at 0° C. was treated dropwise with benzyl carbonochloridate in toluene (8.7 g, 50 wt %, 26 mmol). The mixture was allowed to warm to room temperature and stirred for 3 hours. The reaction mixture was diluted with EtOAc, washed with water and brine, dried (Na₂SO₄), filtered, and concentrated. The resulting residue was purified by flash column chromatography (ethyl acetate/heptane) to afford tert-butyl 6-(2-(((benzyloxy)carbonyl)amino)ethyl)-1,4-oxazepane-4-carboxylate (1.06 g, 2.80 mmol, 14%).

MS (ESI): m/z (+)=379.3.

### Step D: Synthesis of benzyl (2-(1,4-oxazepan-6-yl)ethyl)carbamate

Tert-butyl 6-(2-(((benzyloxy)carbonyl)amino)ethyl)-1,4-oxazepane-4-carboxylate (1.06 g, 2.80 mmol) was treated with HCl in 1,4-dioxane (7.00 mL, 4 M, 28.0 mmol). The mixture was stirred at room temperature for 30 min. The reaction mixture was concentrated and dried under vacuum to afford benzyl (2-(1,4-oxazepan-6-yl)ethyl)carbamate (780 mg, 2.80 mmol, 100%) which was used directly in the next step without purification.

MS (ESI): m/z (+)=279.1.

### Step E: Synthesis of benzyl (2-(4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)ethyl)carbamate

A solution of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (500 mg, 1.14 mmol) and benzyl (2-(1,4-oxazepan-6-yl)ethyl)carbamate (317 mg, 1.14 mmol) in DMSO (4 mL) was treated with DIPEA (736 mg, 992 μL, 5.70 mmol) and warmed at 60° C. for 20 hours. The reaction mixture was purified directly by reverse-phase flash column chromatography using a gradient of 10-100% B (A: water+0.1% formic acid; B: acetonitrile+0.1% formic acid) to afford benzyl (2-(4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)ethyl)carbamate (469 mg, 760 μmol, 66.7%).

MS (ESI): m/z (+)=617.3.

### Step F: Synthesis of methyl 5-(4-amino-6-(4-(6-(2-(((benzyloxy)carbonyl)amino)ethyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-chloro-3-fluorophenyl)pentanoate

A solution of benzyl (2-(4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)ethyl)carbamate (100 mg, 162 μmol), methyl 5-(4-amino-2-chloro-3-fluoro-6-(4,4,5,5-tetramethyl-1,3,2-dioxaborolan-2-yl)phenyl)pentanoate (68.7 mg, 178 μmol), Cs₂CO₃ (158 mg, 486 μmol), and cataCXium A Pd G3 (11.8 mg, 16.2 μmol) in 1,4-dioxane (4 mL) and water (0.5 mL) was warmed at 80° C. for 3 hours. The reaction mixture was concentrated onto celite and purified by reverse-phase flash column chromatography using a gradient of 10-100% B (A: water+0.1% formic acid; B: acetonitrile+0.1% formic acid) to afford methyl 5-(4-amino-6-(4-(6-(2-(((benzyloxy)carbonyl)amino)ethyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-chloro-3-fluorophenyl)pentanoate (54 mg, 64 μmol, 40%).

MS (ESI): m/z (+)=840.4.

### Step G: Synthesis of methyl 5-(4-amino-6-(4-(6-(2-aminoethyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-chloro-3-fluorophenyl)pentanoate

A solution of methyl 5-(4-amino-6-(4-(6-(2-(((benzyloxy)carbonyl)amino)ethyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-chloro-3-fluorophenyl)pentanoate (49 mg, 58 μmol) in THF (4.5 mL) at room temperature was treated with Pd/C (20 mg, 5 wt %, 9.4 μmol) and bubbled with hydrogen gas for 30 minutes. The mixture was stirred under a hydrogen atmosphere at room temperature for 40 hours. The reaction mixture was filtered and concentrated to afford crude methyl 5-(4-amino-6-(4-(6-(2-aminoethyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-chloro-3-fluorophenyl)pentanoate (41 mg, 35 μmol, 60%) which was used directly in the next step without purification.

MS (ESI): m/z (+)=706.5.

### Step H: Synthesis of 5-(4-amino-6-(4-(6-(2-aminoethyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-chloro-3-fluorophenyl)pentanoic acid

A solution of methyl 5-(4-amino-6-(4-(6-(2-aminoethyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-chloro-3-fluorophenyl)pentanoate (41 mg, 58 μmol) in THF (4 mL) and water (1 mL) was treated with LiOH·H₂O (24 mg, 0.58 mmol). The mixture was stirred at room temperature for 2 hours. The reaction mixture was concentrated and purified by reverse-phase flash column chromatography using a gradient of 10-100% B (A: water+0.1% formic acid; B: acetonitrile+0.1% formic acid to afford 5-(4-amino-6-(4-(6-(2-aminoethyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-chloro-3-fluorophenyl)pentanoic acid (12.2 mg, 17.6 μmol, 30%).

MS (ESI): m/z (+)=692.5.

### Step I: Synthesis of 3⁵-amino-3³-chloro-2⁸,3⁴-difluoro-2²-(((2R,7aS)-2-fluorotetrahydro-1H-pyrrolizin-7a(5H)-yl)methoxy)-9-aza-1(4,6)-oxazepana-2(4,7)-pyrido[4,3-d]pyrimidina-3(1,2)-benzenacycloundecaphan-8-one

A solution of 5-(4-amino-6-(4-(6-(2-aminoethyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-chloro-3-fluorophenyl)pentanoic acid (12.3 mg, 17.8 μmol) in DMF (2 mL) was treated with HATU (8.11 mg, 21.3 μmol) and DIPEA (11.5 mg, 16 μL, 88.8 μmol). The mixture was stirred at room temperature for 30 minutes. The reaction mixture was purified by HPLC (Method A) to afford 3⁵-amino-3³-chloro-2⁸,3⁴-difluoro-2²-(((2R,7aS)-2-fluorotetrahydro-1H-pyrrolizin-7a(5H)-yl)methoxy)-9-aza-1(4,6)-oxazepana-2(4,7)-pyrido[4,3-d]pyrimidina-3(1,2)-benzenacycloundecaphan-8-one (1.5 mg, 2.2 μmol, 13%).

MS (ESI): m/z (+)=674.4.

¹H NMR (400 MHz, MeOD) δ 9.17 (s, 1H), 6.77 (d, J=8.4 Hz, 1H), 5.40-5.18 (m, 1H), 4.67-4.57 (m, 1H), 4.57-4.46 (m, 1H), 4.36-4.22 (m, 2H), 4.04-3.86 (m, 2H), 3.76-3.53 (m, 4H), 3.50-3.28 (m, 4H), 3.18-3.00 (m, 4H), 2.66-2.53 (m, 1H), 2.47-1.83 (m, 8H), 1.61-1.15 (m, 6H).

### Example 53. Synthesis of 1⁶-amino-1²-chloro-2⁸-fluoro-2²-(((2R,7aS)-2-fluorotetrahydro-1H-pyrrolizin-7a(5H)-yl)methoxy)-6-aza-2(7,4)-pyrido[4,3-d]pyrimidina-1(4,3)-pyridina-3(1,3)-piperidinacycloundecaphan-7-one (Compound 186a)

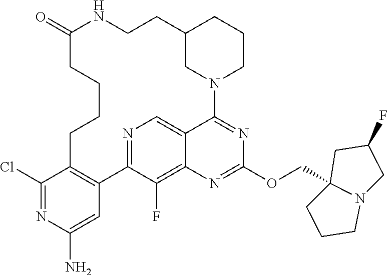 [SMILES: Nc1cc2c(c(Cl)n1)CCCCC(=O)NCCC1CCCN(C1)c1nc(OC[C@@]34CCCN3C[C@H](F)C4)nc3c(F)c-2ncc13]

1⁶-amino-1²-chloro-2⁸-fluoro-2²-(((2R,7aS)-2-fluorotetrahydro-1H-pyrrolizin-7a(5H)-yl)methoxy)-6-aza-2(7,4)-pyrido[4,3-d]pyrimidina-1(4,3)-pyridina-3(1,3)-piperidinacycloundecaphan-7-one was prepared in an analogous manner to Example 29 by using methyl 5-(6-(bis(4-methoxybenzyl)amino)-2-chloro-4-(4,4,5,5-tetramethyl-1,3,2-dioxaborolan-2-yl)pyridin-3-yl)pentanoate in Step A.

MS (ESI): m/z (+)=641.3.

¹H NMR (400 MHz, MeOD) δ 9.01 (s, 1H), 6.54 (s, 1H), 5.39-5.21 (m, 1H), 4.80-4.71 (m, 1H), 4.60-4.52 (m, 1H), 4.37-4.21 (m, 2H), 3.45-3.11 (m, 8H), 3.09-2.93 (m, 2H), 2.59-2.50 (m, 1H), 2.39-2.07 (m, 4H), 2.07-1.94 (m, 2H), 1.94-1.62 (m, 6H), 1.61-1.33 (m, 4H), 1.29-1.13 (m, 2H).

### Example 54. Synthesis of 1⁶-amino-1²-chloro-2⁸-fluoro-2²-(((2R,7aS)-2-fluorotetrahydro-1H-pyrrolizin-7a(5H)-yl)methoxy)-3³-hydroxy-6-aza-2(7,4)-quinazolina-1(4,3)-pyridina-3(1,3)-piperidinacycloundecaphan-7-one (Compound 187a)

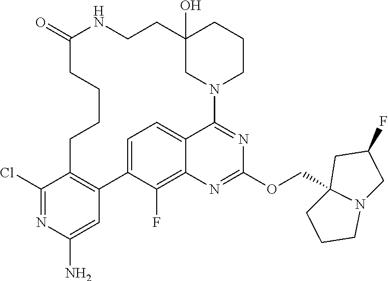 [SMILES: Nc1cc2c(c(Cl)n1)CCCCC(=O)NCCC1(O)CCCN(C1)c1nc(OC[C@@]34CCCN3C[C@H](F)C4)nc3c(F)c-2ccc13]

1⁶-amino-1²-chloro-2⁸-fluoro-2²-(((2R,7aS)-2-fluorotetrahydro-1H-pyrrolizin-7a(5H)-yl)methoxy)-3³-hydroxy-6-aza-2(7,4)-quinazolina-1(4,3)-pyridina-3(1,3)-piperidinacycloundecaphan-7-one was prepared in an analogous manner to Example 41 by using tert-butyl (2-(3-hydroxypiperidin-3-yl)ethyl)carbamate in Step A and methyl 5-(6-(bis(4-methoxybenzyl)amino)-2-chloro-4-(4,4,5,5-tetramethyl-1,3,2-dioxaborolan-2-yl)pyridin-3-yl)pentanoate in Step C.

LCMS: Retention time=1.94 min, m/z (+)=656.4.

### Example 55. Synthesis of 3³-amino-2⁸,3⁷-difluoro-2²-(((2R,7aS)-2-fluorotetrahydro-1H-pyrrolizin-7a(5H)-yl)methoxy)-8-aza-2(4,7)-pyrido[4,3-d]pyrimidina-1(1,3)-piperidina-3(1,8)-naphthalenacyclodecaphan-7-one (Compound 189a)

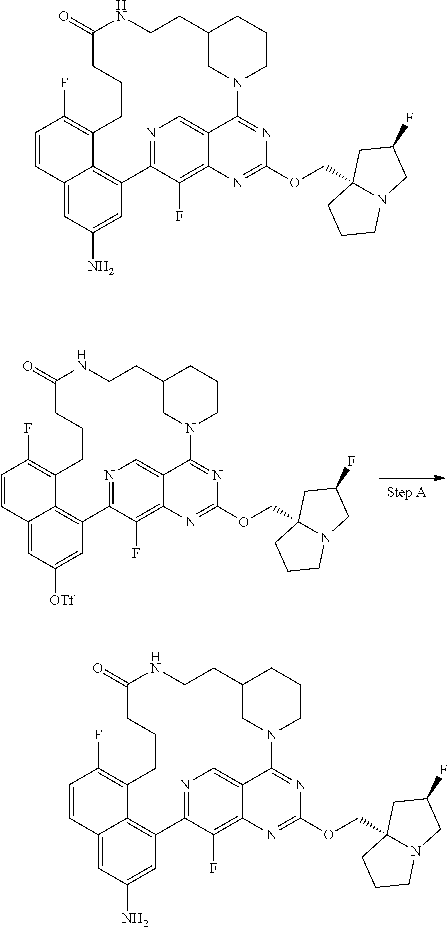 [SMILES: Nc1cc2c3c(c(F)ccc3c1)CCCC(=O)NCCC1CCCN(C1)c1nc(OC[C@@]34CCCN3C[C@H](F)C4)nc3c(F)c-2ncc13 ; O=C1CCCc2c(F)ccc3cc(OS(=O)(=O)C(F)(F)F)cc(c23)-c2ncc3c(nc(OC[C@@]45CCCN4C[C@H](F)C5)nc3c2F)N2CCCC(CCN1)C2 ; Nc1cc2c3c(c(F)ccc3c1)CCCC(=O)NCCC1CCCN(C1)c1nc(OC[C@@]34CCCN3C[C@H](F)C4)nc3c(F)c-2ncc13]

### Step A: Synthesis of 3³-amino-2⁸,3⁷-difluoro-2²-(((2R,7aS)-2-fluorotetrahydro-1H-pyrrolizin-7a(5H)-yl)methoxy)-8-aza-2(4,7)-pyrido[4,3-d]pyrimidina-1(1,3)-piperidina-3(1,8)-naphthalenacyclodecaphan-7-one

A solution of 2⁸,3⁷-difluoro-2²-(((2R,7aS)-2-fluorotetrahydro-1H-pyrrolizin-7a(5H)-yl)methoxy)-7-oxo-8-aza-2(4,7)-pyrido[4,3-d]pyrimidina-1(1,3)-piperidina-3(1,8)-naphthalenacyclodecaphane-3³-yl trifluoromethanesulfonate (7.7 mg, 9.7 μmol), tert-butyl carbamate (2.8 mg, 24 μmol), tBuBrettPhosPdG3 (1.6 mg, 1.9 μmol), and Cs₂CO₃ (9.5 mg, 29 μmol) in tert-Amyl alcohol (1 mL) was degassed with nitrogen. The mixture was stirred at 110° C. for 3 hours. The reaction mixture was filtered and purified by HPLC (Method A) to afford tert-butyl (2⁸,3⁷-difluoro-2²-(((2R,7aS)-2-fluorotetrahydro-1H-pyrrolizin-7a(5H)-yl)methoxy)-7-oxo-8-aza-2(4,7)-pyrido[4,3-d]pyrimidina-1(1,3)-piperidina-3(1,8)-naphthalenacyclodecaphane-3³-yl)carbamate (1.3 mg, 1.7 μmol, 18%). The residue was taken up in DCM (0.5 mL) and was treated with HCl in 1,4-dioxane (0.24 mL, 4 M, 0.97 mmol). The mixture was stirred at room temperature for 3 hours. The volatiles were removed under a stream of nitrogen and was further dried under vacuum to afford 3³-amino-2⁸,3⁷-difluoro-2²-(((2R,7aS)-2-fluorotetrahydro-1H-pyrrolizin-7a(5H)-yl)methoxy)-8-aza-2(4,7)-pyrido[4,3-d]pyrimidina-1(1,3)-piperidina-3(1,8)-naphthalenacyclodecaphan-7-one (0.8 mg, 1 μmol, 10%).

MS (ESI): m/z (+)=660.4.

### Example 56. Synthesis of 3³-amino-2⁸,3⁷-difluoro-2²-(((2R,7aS)-2-fluorotetrahydro-1H-pyrrolizin-7a(5H)-yl)methoxy)-8-aza-2(4,7)-quinazolina-1(1,3)-piperidina-3(1,8)-naphthalenacyclodecaphan-7-one (Compound 190a)

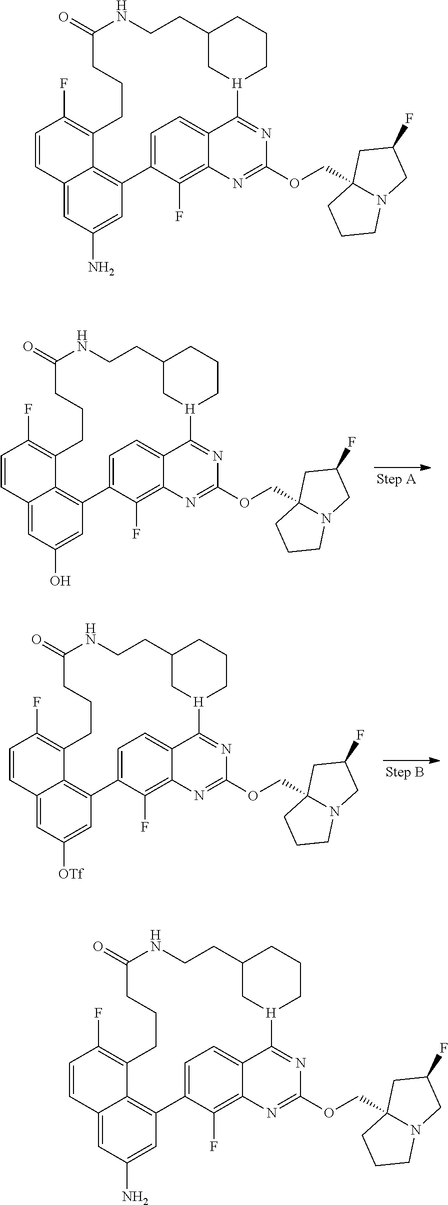

Step A: Synthesis of 2⁸,3⁷-difluoro-2²-(((2R,7aS)-2-fluorotetrahydro-1H-pyrrolizin-7a(5H)-yl)methoxy)-7-oxo-8-aza-2(4,7)-quinazolina-1(1,3)-piperidina-3(1,8)-naphthalenacyclodecaphane-3³-yl trifluoromethanesulfonate

A solution of 2⁸,3⁷-difluoro-2²-(((2R,7aS)-2-fluorotetrahydro-1H-pyrrolizin-7a(5H)-yl)methoxy)-3³-hydroxy-8-aza-2(4,7)-quinazolina-1(1,3)-piperidina-3(1,8)-naphthalenacyclodecaphan-7-one (32 mg, 49 μmol) and DIPEA (19 mg, 25 μL, 0.15 mmol) in DCM (1 mL) was treated with trifluoromethanosulfonic anhydride (21 mg, 12 μL, 73 μmol). The mixture was stirred for 30 minutes. The reaction mixture was concentrated and purified by reverse-phase flash column chromatography using a gradient of 10-1⁰⁰% B (A: water+0.1% formic acid; B: acetonitrile+0.1% formic acid) to afford 2⁸,3⁷-difluoro-2²-(((2R,7aS)-2-fluorotetrahydro-1H-pyrrolizin-7a(5H)-yl)methoxy)-7-oxo-8-aza-2(4,7)-quinazolina-1(1,3)-piperidina-3(1,8)-naphthalenacyclodecaphane-3³-yl trifluoromethanesulfonate (14.3 mg, 18.1 μmol, 37%).

MS (ESI): m/z (+)=792.3.

### Step B: Synthesis of 3³-amino-2⁸,3⁷-difluoro-2²-(((2R,7aS)-2-fluorotetrahydro-1H-pyrrolizin-7a(5H)-yl)methoxy)-8-aza-2(4,7)-quinazolina-1(1,3)-piperidina-3(1,8)-naphthalenacyclodecaphan-7-one

A solution of 2⁸,3⁷-difluoro-2²-(((2R,7aS)-2-fluorotetrahydro-1H-pyrrolizin-7a(5H)-yl)methoxy)-7-oxo-8-aza-2(4,7)-quinazolina-1(1,3)-piperidina-3(1,8)-naphthalenacyclodecaphane-3³-yl trifluoromethanesulfonate (14.3 mg, 18.1 μmol), tert-butyl carbamate (5.29 mg, 45.1 μmol), tBuBrettPhosPdG3 (2.92 mg, 3.61 μmol), and Cs₂CO₃ (17.7 mg, 54.2 μmol) in tert-Amyl alcohol (2 mL) was degassed with nitrogen. The mixture was stirred at 110° C. for 3 hours. The reaction mixture was filtered and purified by HPLC to afford tert-butyl (2⁸,3⁷-difluoro-2²-(((2R,7aS)-2-fluorotetrahydro-1H-pyrrolizin-7a(5H)-yl)methoxy)-7-oxo-8-aza-2(4,7)-quinazolina-1(1,3)-piperidina-3(1,8)-naphthalenacyclodecaphane-3³-yl)carbamate (1.5 mg, 2.0 μmol, 11%). The resulting residue was taken up in DCM (0.5 mL) and was treated with HCl in 1,4-dioxane (65.8 mg, 451 μL, 4 M, 1.81 mmol). The mixture was stirred at room temperature for 3 hours. The volatiles were removed under a stream of nitrogen, and the resulting residue was purified by HPLC (Method A) to afford 3³-amino-2⁸,3⁷-difluoro-2²-(((2R,7aS)-2-fluorotetrahydro-1H-pyrrolizin-7a(5H)-yl)methoxy)-8-aza-2(4,7)-quinazolina-1(1,3)-piperidina-3(1,8)-naphthalenacyclodecaphan-7-one (1.0 mg, 1.5 μmol, 8.4%).

LCMS: Retention time=2.15 min, m/z (+)=659.4.

### Example 57. Synthesis of 2⁸,3⁷-difluoro-2²-(((2R,7aS)-2-fluorotetrahydro-1H-pyrrolizin-7a(5H)-yl)methoxy)-8-aza-2(4,7)-quinazolina-1(1,3)-piperidina-3(1,8)-naphthalenacyclodecaphan-7-one (Compound 191a)

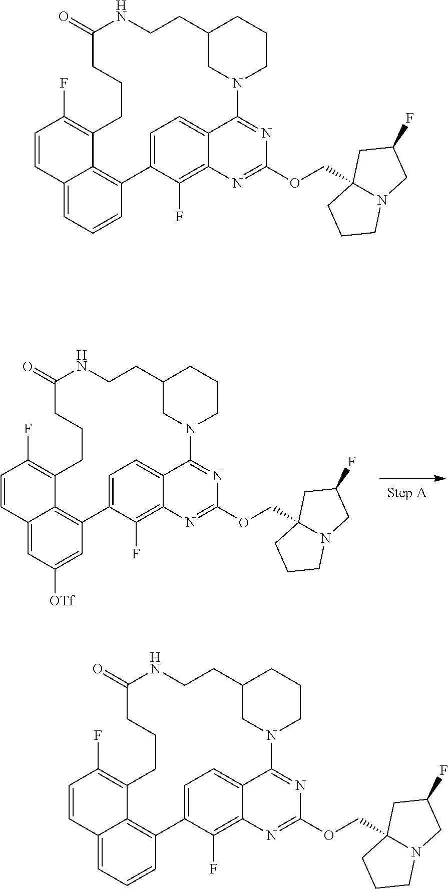 [SMILES: O=C1CCCc2c(F)ccc3cccc(c23)-c2ccc3c(nc(OC[C@@]45CCCN4C[C@H](F)C5)nc3c2F)N2CCCC(CCN1)C2 ; O=C1CCCc2c(F)ccc3cc(OS(=O)(=O)C(F)(F)F)cc(c23)-c2ccc3c(nc(OC[C@@]45CCCN4C[C@H](F)C5)nc3c2F)N2CCCC(CCN1)C2 ; O=C1CCCc2c(F)ccc3cccc(c23)-c2ccc3c(nc(OC[C@@]45CCCN4C[C@H](F)C5)nc3c2F)N2CCCC(CCN1)C2]

### Step A: Synthesis of 2⁸,3⁷-difluoro-2²-(((2R,7aS)-2-fluorotetrahydro-1H-pyrrolizin-7a(5H)-yl)methoxy)-8-aza-2(4,7)-quinazolina-1(1,3)-piperidina-3(1,8)-naphthalenacyclodecaphan-7-one

A solution of 2⁸,3⁷-difluoro-2²-(((2R,7aS)-2-fluorotetrahydro-1H-pyrrolizin-7a(5H)-yl)methoxy)-7-oxo-8-aza-2(4,7)-quinazolina-1(1,3)-piperidina-3(1,8)-naphthalenacyclodecaphane-3³-yl trifluoromethanesulfonate (14.3 mg, 18.1 μmol), tert-butyl carbamate (5.29 mg, 45.1 μmol), tBuBrettPhosPdG3 (2.92 mg, 3.61 μmol), and Cs₂CO₃ (17.7 mg, 54.2 μmol) in tert-Amyl alcohol (2 mL) was degassed with nitrogen. The mixture was stirred at 110° C. for 3 hours. The reaction mixture was filtered and purified by HPLC (Method A) to afford 2⁸,3⁷-difluoro-2²-(((2R,7aS)-2-fluorotetrahydro-1H-pyrrolizin-7a(5H)-yl)methoxy)-8-aza-2(4,7)-quinazolina-1(1,3)-piperidina-3(1,8)-naphthalenacyclodecaphan-7-one (2.0 mg, 3.1 μmol, 17%).

MS (ESI): m/z (+)=644.4.

¹H NMR (400 MHz, MeOD) δ 7.96-7.91 (m, 1H), 7.90-7.82 (m, 1H), 7.76-7.69 (m, 1H), 7.47-7.41 (m, 1H), 7.32-7.20 (m, 3H), 5.39-5.20 (m, 1H), 4.76-4.67 (m, 1H), 4.52-4.39 (m, 1H), 4.37-4.17 (m, 1H), 3.58-3.46 (m, 1H), 3.43-3.16 (m, 5H), 3.12-2.96 (m, 2H), 2.79-2.67 (m, 2H), 2.43-1.67 (m, 12H), 1.65-1.21 (m, 4H), 1.13-0.96 (m, 2H).

### Example 58. Synthesis of 3³-amino-2⁸,3⁷-difluoro-2²-(((2R,7aS)-2-fluorotetrahydro-1H-pyrrolizin-7a(5H)-yl)methoxy)-8-aza-1(4,6)-oxazepana-2(4,7)-pyrido[4,3-d]pyrimidina-3(1,8)-naphthalenacyclononaphan-7-one (Compound 192a)

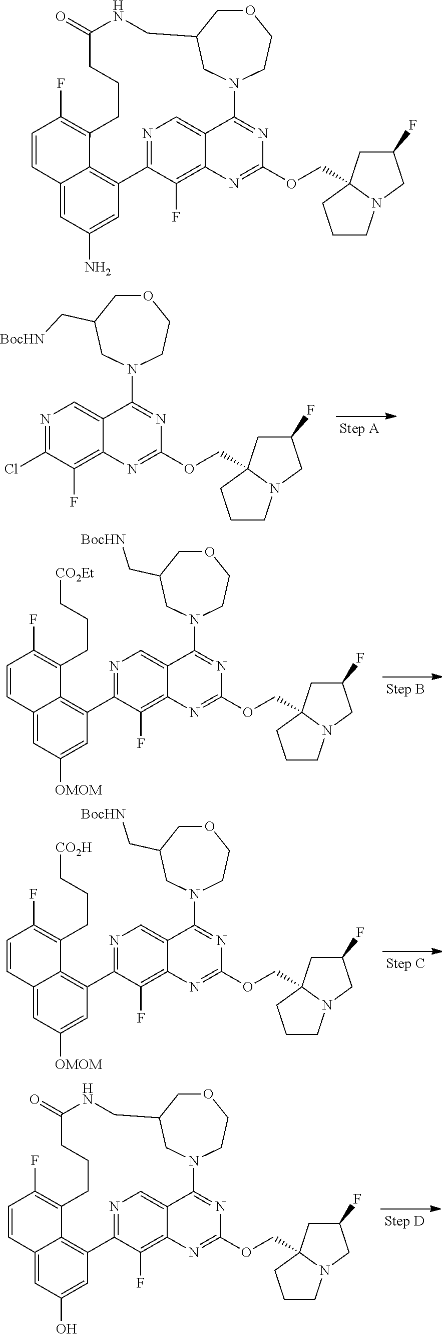

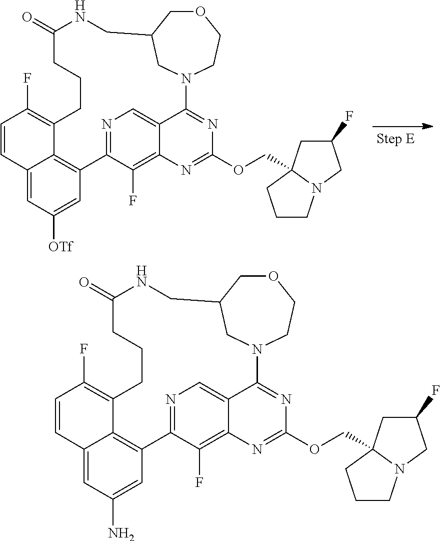 [SMILES: O=C1CCCc2c(F)ccc3cc(OS(=O)(=O)C(F)(F)F)cc(c23)-c2ncc3c(nc(OC[C@@]45CCCN4C[C@H](F)C5)nc3c2F)N2CCOCC(CN1)C2 ; Nc1cc2c3c(c(F)ccc3c1)CCCC(=O)NCC1COCCN(C1)c1nc(OC[C@@]34CCCN3C[C@H](F)C4)nc3c(F)c-2ncc13]

### Step A: Synthesis of ethyl 4-(8-(4-(6-(((tert-butoxycarbonyl)amino)methyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoate

A solution of tert-butyl ((4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)methyl)carbamate (180 mg, 316 μmol), ethyl 4-(2-fluoro-6-(methoxymethoxy)-8-(4,4,5,5-tetramethyl-1,3,2-dioxaborolan-2-yl)naphthalen-1-yl)butanoate (169 mg, 380 μmol), Cs₂CO₃ (309 mg, 949 μmol), and cataCXium A Pd G3 (23.0 mg, 31.6 μmol) in 1,4-dioxane (6 mL) and water (1 mL) was degassed with nitrogen and was warmed at 80° C. for 16 hours. The reaction mixture was concentrated and purified by reverse-phase flash column chromatography using a gradient of 10-100% B (A: water+0.1% formic acid; B: acetonitrile+0.1% formic acid) to afford ethyl 4-(8-(4-(6-(((tert-butoxycarbonyl)amino)methyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoate (154 mg, 181 μmol, 57.1%).

MS (ESI): m/z (+)=853.5.

### Step B: Synthesis of 4-(8-(4-(6-(((tert-butoxycarbonyl)amino)methyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoic acid

A solution of ethyl 4-(8-(4-(6-(((tert-butoxycarbonyl)amino)methyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoate (154 mg, 181 μmol) in THF (6 mL) and water (2 mL) was treated with LiOH·H₂O (75.8 mg, 1.81 mmol). The mixture was stirred at room temperature for 18 hours. The reaction mixture was concentrated and purified by reverse-phase flash column chromatography using a gradient of 10-100% B (A: water+0.1% formic acid; B: acetonitrile+0.1% formic acid) to afford 4-(8-(4-(6-(((tert-butoxycarbonyl)amino)methyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoic acid (104 mg, 126 μmol, 69.8%).

MS (ESI): m/z (+)=825.5.

### Step C: Synthesis of 2⁸,3⁷-difluoro-2²-(((2R,7aS)-2-fluorotetrahydro-1H-pyrrolizin-7a(5H)-yl)methoxy)-3³-hydroxy-8-aza-1(4,6)-oxazepana-2(4,7)-pyrido[4,3-d]pyrimidina-3(1,8)-naphthalenacyclononaphan-7-one

A solution of 4-(8-(4-(6-(((tert-butoxycarbonyl)amino)methyl)-1,4-oxazepan-4-yl)-8-fluoro-2-(((2R,7aS)-2-fluorotetrahydro-1H-pyrrolizin-7a(5H)-yl)methoxy)pyrido[4,3-d]pyrimidin-7-yl)-2-fluoro-6-(methoxymethoxy)naphthalen-1-yl)butanoic acid (104 mg, 126 μmol) in DCM (1 mL) was treated with HCl in 1,4-dioxane (230 mg, 1.58 mL, 4 M, 6.30 mmol). The mixture was stirred at room temperature for 30 minutes. The volatiles were removed under a stream of nitrogen and the residue was further dried under vacuum. The resulting residue was taken up in DMF (5 mL) and treated with HATU (57.5 mg, 151 μmol) and DIPEA (81.5 mg, 0.11 mL, 630 μmol). The mixture was stirred at room temperature for 60 minutes. The reaction mixture was purified by HPLC (Method A) to afford 2⁸,3⁷-difluoro-2²-(((2R,7aS)-2-fluorotetrahydro-1H-pyrrolizin-7a(5H)-yl)methoxy)-3³-hydroxy-8-aza-1(4,6)-oxazepana-2(4,7)-pyrido[4,3-d]pyrimidina-3(1,8)-naphthalenacyclononaphan-7-one (30.8 mg, 46.5 μmol, 36.9%).

MS (ESI) m/z (+)=663.4.

### Step D: Synthesis of 2⁸,3⁷-difluoro-2²-(((2R,7aS)-2-fluorotetrahydro-1H-pyrrolizin-7a(5H)-yl)methoxy)-7-oxo-8-aza-1(4,6)-oxazepana-2(4,7)-pyrido[4,3-d]pyrimidina-3(1,8)-naphthalenacyclononaphane-3³-yl trifluoromethanesulfonate

A solution of 2⁸,3⁷-difluoro-2²-(((2R,7aS)-2-fluorotetrahydro-1H-pyrrolizin-7a(5H)-yl)methoxy)-3³-hydroxy-8-aza-1(4,6)-oxazepana-2(4,7)-pyrido[4,3-d]pyrimidina-3(1,8)-naphthalenacyclononaphan-7-one (28 mg, 42 μmol) and DIPEA (16 mg, 22 μL, 0.13 mmol) in DCM (1 mL) was treated with trifluoromethanosulfonic anhydride (18 mg, 11 μL, 63 μmol). The mixture was stirred for 30 minutes. The reaction mixture was concentrated and purified by reverse-phase flash column chromatography using a gradient of 10-100% B (A: water+0.1% formic acid; B: acetonitrile+0.1% formic acid) to afford 2⁸,3⁷-difluoro-2²-(((2R,7aS)-2-fluorotetrahydro-1H-pyrrolizin-7a(5H)-yl)methoxy)-7-oxo-8-aza-1(4,6)-oxazepana-2(4,7)-pyrido[4,3-d]pyrimidina-3(1,8)-naphthalenacyclononaphane-3³-yl trifluoromethanesulfonate (17.5 mg, 22.0 μmol, 52%).

MS (ESI): m/z (+)=795.4.

### Step E: Synthesis of 3³-amino-2⁸,3⁷-difluoro-2²-(((2R,7aS)-2-fluorotetrahydro-1H-pyrrolizin-7a(5H)-yl)methoxy)-8-aza-1(4,6)-oxazepana-2(4,7)-pyrido[4,3-d]pyrimidina-3(1,8)-naphthalenacyclononaphan-7-one

A solution of 2⁸,3⁷-difluoro-2²-(((2R,7aS)-2-fluorotetrahydro-1H-pyrrolizin-7a(5H)-yl)methoxy)-7-oxo-8-aza-1(4,6)-oxazepana-2(4,7)-pyrido[4,3-d]pyrimidina-3(1,8)-naphthalenacyclononaphane-3³-yl trifluoromethanesulfonate (17.5 mg, 22.0 μmol), tert-butyl carbamate (6.45 mg, 55.0 μmol), tBuBrettPhosG3 (3.56 mg, 4.40 μmol), and Cs₂CO₃ (21.5 mg, 66.1 μmol) in tert-Amyl alcohol (2 mL) was degassed with nitrogen. The mixture was stirred at 110° C. for 3 hours. The reaction mixture was filtered and purified by HPLC to afford tert-butyl (2⁸,3⁷-difluoro-2²-(((2R,7aS)-2-fluorotetrahydro-1H-pyrrolizin-7a(5H)-yl)methoxy)-7-oxo-8-aza-1(4,6)-oxazepana-2(4,7)-pyrido[4,3-d]pyrimidina-3(1,8)-naphthalenacyclononaphane-3³-yl)carbamate (8.4 mg, 11 μmol, 50%). The material was taken up in DCM (0.5 mL) and treated with HCl in 1,4-dioxane (80.3 mg, 550 μL, 4 M, 2.20 mmol). The mixture was stirred at room temperature for 3 hours. The volatiles were removed under a stream of nitrogen, and the resulting residue was purified by HPLC (Method A) to afford 3³-amino-2⁸,3⁷-difluoro-2²-(((2R,7aS)-2-fluorotetrahydro-1H-pyrrolizin-7a(5H)-yl)methoxy)-8-aza-1(4,6)-oxazepana-2(4,7)-pyrido[4,3-d]pyrimidina-3(1,8)-naphthalenacyclononaphan-7-one (2.2 mg, 3.3 μmol, 15%).

MS (ESI): m/z (+)=662.4.

¹H NMR (400 MHz, MeOD) δ 9.04 (s, 1H), 7.57-7.51 (m, 1H), 7.18-7.10 (m, 2H), 6.98-6.94 (m, 1H), 5.53-5.31 (m, 1H), 4.71-4.63 (m, 1H), 4.58-4.43 (m, 2H), 4.37-4.25 (m, 2H), 4.17-3.48 (m, 8H), 3.36-3.25 (m, 2H), 2.85-2.74 (m, 1H), 2.66-1.79 (m, 9H), 1.57-1.18 (m, 4H).

### Example 59. Synthesis of 2⁸,3⁷-difluoro-2²-(((2R,7aS)-2-fluorotetrahydro-1H-pyrrolizin-7a(5H)-yl)methoxy)-8-aza-1(4,6)-oxazepana-2(4,7)-pyrido[4,3-d]pyrimidina-3(1,8)-naphthalenacyclononaphan-7-one (Compound 193a)

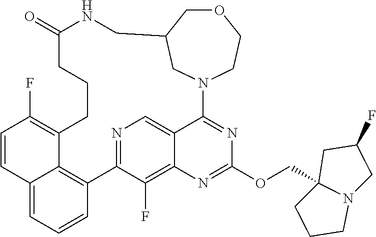 [SMILES: O=C1CCCc2c(F)ccc3cccc(c23)-c2ncc3c(nc(OC[C@@]45CCCN4C[C@H](F)C5)nc3c2F)N2CCOCC(CN1)C2]

2⁸,3⁷-difluoro-2²-(((2R,7aS)-2-fluorotetrahydro-1H-pyrrolizin-7a(5H)-yl)methoxy)-8-aza-1(4,6)-oxazepana-2(4,7)-pyrido[4,3-d]pyrimidina-3(1,8)-naphthalenacyclononaphan-7-one was prepared in an analogous manner to Example 37 by using tert-butyl ((4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)methyl)carbamate in Step A.

MS (ESI): m/z (+)=647.4.

¹H NMR (400 MHz, MeOD) δ 9.08 (s, 1H), 8.03-7.98 (m, 1H), 7.93-7.85 (m, 1H), 7.62-7.40 (m, 2H), 7.36-7.28 (m, 1H), 5.53-5.35 (m, 1H), 4.71-4.63 (m, 1H), 4.60-4.47 (m, 2H), 4.38-4.27 (m, 2H), 4.10-3.57 (m, 8H), 3.35-3.27 (m, 2H), 2.83-2.73 (m, 1H), 2.69-1.84 (m, 9H), 1.60-1.40 (m, 4H).

### Example 60. Synthesis of 2⁸,3⁷-difluoro-2²-(((2R,7aS)-2-fluorotetrahydro-1H-pyrrolizin-7a(5H)-yl)methoxy)-3³-hydroxy-8-aza-1(4,6)-oxazepana-2(4,7)-pyrido[4,3-d]pyrimidina-3(1,8)-naphthalenacyclodecaphan-7-one (Compound 194a)

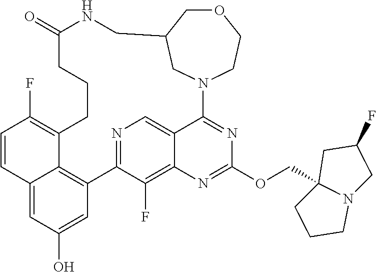 [SMILES: O=C1CCCc2c(F)ccc3cc(O)cc(c23)-c2ncc3c(nc(OC[C@@]45CCCN4C[C@H](F)C5)nc3c2F)N2CCOCC(CN1)C2]

2⁸,3⁷-difluoro-2²-(((2R,7aS)-2-fluorotetrahydro-1H-pyrrolizin-7a(5H)-yl)methoxy)-3³-hydroxy-8-aza-1(4,6)-oxazepana-2(4,7)-pyrido[4,3-d]pyrimidina-3(1,8)-naphthalenacyclodecaphan-7-one was prepared in an analogous manner to Example 52 by using ethyl 4-(2-fluoro-6-(methoxymethoxy)-8-(4,4,5,5-tetramethyl-1,3,2-dioxaborolan-2-yl)naphthalen-1-yl)butanoate in Step F.

LCMS: Retention time=2.00 min, m/z (+)=677.5.

### Example 61. Synthesis of 2⁸,3⁷-difluoro-2²-(((2R,7aS)-2-fluorotetrahydro-1H-pyrrolizin-7a(5H)-yl)methoxy)-3³-hydroxy-8-aza-1(4,6)-oxazepana-2(4,7)-quinazolina-3(1,8)-naphthalenacyclodecaphan-7-one (Compound 195a)

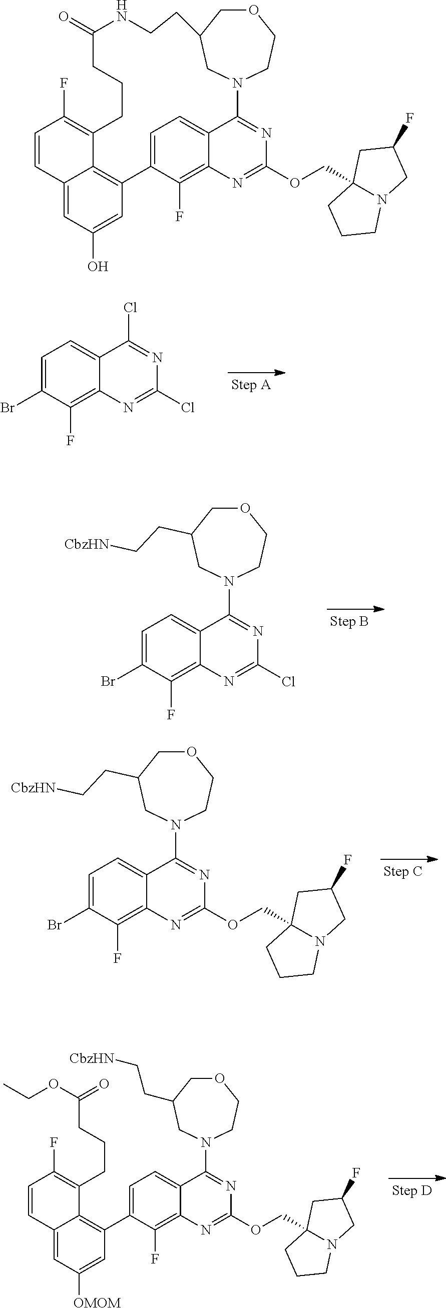

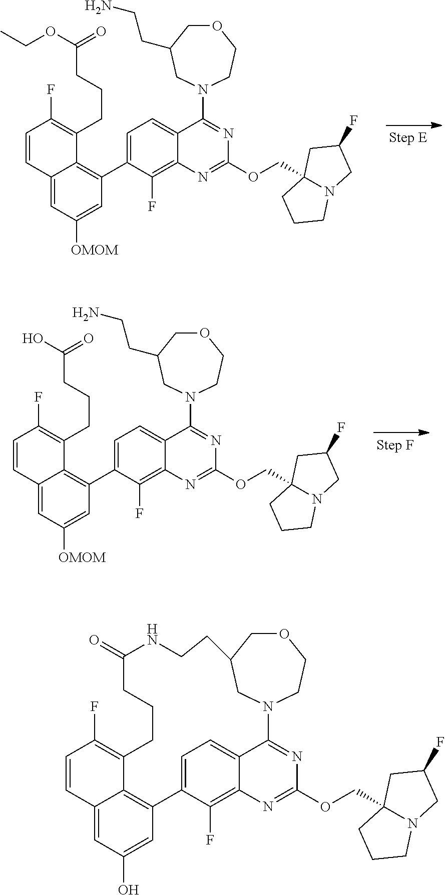

### Step A: Synthesis of benzyl (2-(4-(7-bromo-2-chloro-8-fluoroquinazolin-4-yl)-1,4-oxazepan-6-yl)ethyl)carbamate

A solution of 7-bromo-2,4-dichloro-8-fluoroquinazoline (275 mg, 929 μmol) in DCM (5 mL) under nitrogen was cooled to −78° C. Benzyl (2-(1,4-oxazepan-6-yl)ethyl)carbamate (259 mg, 929 μmol) was added, and the reaction was stirred for 5 minutes. DIPEA (601 mg, 809 μL, 4.65 mmol) was added dropwise. The resulting reaction mixture was stirred at −78 C for 10 minutes and then allowed to warm to room temperature. The mixture was stirred for 1 hour. The reaction mixture was purified directly by flash column chromatography (ethyl acetate/heptane) to afford benzyl (2-(4-(7-bromo-2-chloro-8-fluoroquinazolin-4-yl)-1,4-oxazepan-6-yl)ethyl)carbamate (445 mg, 827 μmol, 89.0%).

MS (ESI): m/z (+)=537.2.

### Step B: Synthesis of benzyl (2-(4-(7-bromo-8-fluoro-2-(((2R,7aS)-2-fluorotetrahydro-1H-pyrrolizin-7a(5H)-yl)methoxy)quinazolin-4-yl)-1,4-oxazepan-6-yl)ethyl)carbamate

A solution of ((2R,7aS)-2-fluorotetrahydro-1H-pyrrolizin-7a(5H)-yl)methanol (263 mg, 1.65 mmol), benzyl (2-(4-(7-bromo-2-chloro-8-fluoroquinazolin-4-yl)-1,4-oxazepan-6-yl)ethyl)carbamate (445 mg, 827 μmol), cataCXium A Pd G3 (90.4 mg, 124 μmol), and Cs₂CO₃ (809 mg, 2.48 mmol) in 1,4-dioxane (10 mL) was degassed with nitrogen and warmed at 70° C. for 2 hours. ((2R,7aS)-2-fluorotetrahydro-1H-pyrrolizin-7a(5H)-yl)methanol (263 mg, 2 equiv., 1.65 mmol) was added and the reaction mixture was stirred at 70° C. for 2 hours. The reaction mixture was concentrated onto celite and purified by reverse-phase flash column chromatography using a gradient of 10-100% B (A: water+0.1% formic acid; B: acetonitrile+0.1% formic acid) to afford benzyl (2-(4-(7-bromo-8-fluoro-2-(((2R,7aS)-2-fluorotetrahydro-1H-pyrrolizin-7a(5H)-yl)methoxy)quinazolin-4-yl)-1,4-oxazepan-6-yl)ethyl)carbamate (93 mg, 0.14 mmol, 17%).

MS (ESI): m/z (+)=660.2.

### Step C: Synthesis of ethyl 4-(8-(4-(6-(2-(((benzyloxy)carbonyl)amino)ethyl)-1,4-oxazepan-4-yl)-8-fluoro-2-(((2R,7aS)-2-fluorotetrahydro-1H-pyrrolizin-7a(5H)-yl)methoxy)quinazolin-7-yl)-2-fluoro-6-(methoxymethoxy)naphthalen-1-yl)butanoate

A solution of benzyl (2-(4-(7-bromo-8-fluoro-2-(((2R,7aS)-2-fluorotetrahydro-1H-pyrrolizin-7a(5H)-yl)methoxy)quinazolin-4-yl)-1,4-oxazepan-6-yl)ethyl)carbamate (92 mg, 0.14 mmol), ethyl 4-(2-fluoro-6-(methoxymethoxy)-8-(4,4,5,5-tetramethyl-1,3,2-dioxaborolan-2-yl)naphthalen-1-yl)butanoate (62 mg, 0.14 mmol), Cs₂CO₃ (0.14 g, 0.42 mmol), and cataCXium A Pd G3 (10 mg, 14 μmol) in 1,4-dioxane (4 mL) and water (0.5 mL) was warmed at 80° C. for 2 hours. The reaction mixture was concentrated and was purified by reverse-phase flash column chromatography using a gradient of 10-100% B (A: water+0.1% formic acid; B: acetonitrile+0.1% formic acid) to afford ethyl 4-(8-(4-(6-(2-(((benzyloxy)carbonyl)amino)ethyl)-1,4-oxazepan-4-yl)-8-fluoro-2-(((2R,7aS)-2-fluorotetrahydro-1H-pyrrolizin-7a(5H)-yl)methoxy)quinazolin-7-yl)-2-fluoro-6-(methoxymethoxy)naphthalen-1-yl)butanoate (98 mg, 0.11 mmol, 78%).

MS (ESI): m/z (+)=900.5.

### Step D: Synthesis of ethyl 4-(8-(4-(6-(2-aminoethyl)-1,4-oxazepan-4-yl)-8-fluoro-2-(((2R,7aS)-2-fluorotetrahydro-1H-pyrrolizin-7a(5H)-yl)methoxy)quinazolin-7-yl)-2-fluoro-6-(methoxymethoxy)naphthalen-1-yl)butanoate

A solution of ethyl 4-(8-(4-(6-(2-(((benzyloxy)carbonyl)amino)ethyl)-1,4-oxazepan-4-yl)-8-fluoro-2-(((2R,7aS)-2-fluorotetrahydro-1H-pyrrolizin-7a(5H)-yl)methoxy)quinazolin-7-yl)-2-fluoro-6-(methoxymethoxy)naphthalen-1-yl)butanoate (98 mg, 0.11 mmol) in THF (5 mL) was treated with Pd/C (93 mg, 5 wt %, 44 μmol) and bubbled with hydrogen. The mixture was stirred under a hydrogen atmosphere at room temperature for 18 hours. The reaction mixture was filtered and concentrated to afford ethyl 4-(8-(4-(6-(2-aminoethyl)-1,4-oxazepan-4-yl)-8-fluoro-2-(((2R,7aS)-2-fluorotetrahydro-1H-pyrrolizin-7a(5H)-yl)methoxy)quinazolin-7-yl)-2-fluoro-6-(methoxymethoxy)naphthalen-1-yl)butanoate (83 mg, 0.11 mmol, 100%) MS (ESI): m/z (+)=766.5.

### Step E: Synthesis of 4-(8-(4-(6-(2-aminoethyl)-1,4-oxazepan-4-yl)-8-fluoro-2-(((2R,7aS)-2-fluorotetrahydro-1H-pyrrolizin-7a(5H)-yl)methoxy)quinazolin-7-yl)-2-fluoro-6-(methoxymethoxy)naphthalen-1-yl)butanoic acid

A solution of ethyl 4-(8-(4-(6-(2-aminoethyl)-1,4-oxazepan-4-yl)-8-fluoro-2-(((2R,7aS)-2-fluorotetrahydro-1H-pyrrolizin-7a(5H)-yl)methoxy)quinazolin-7-yl)-2-fluoro-6-(methoxymethoxy)naphthalen-1-yl)butanoate (80 mg, 0.10 mmol) in THF (5 mL) and water (1.5 mL) was treated with LiOH·H₂O (44 mg, 1.0 mmol). The mixture was stirred at room temperature for 20 hours. The reaction mixture was concentrated and purified by reverse-phase flash column chromatography using a gradient of 10-100% B (A: water+0.1% formic acid; B: acetonitrile+0.1% formic acid to afford 4-(8-(4-(6-(2-aminoethyl)-1,4-oxazepan-4-yl)-8-fluoro-2-(((2R,7aS)-2-fluorotetrahydro-1H-pyrrolizin-7a(5H)-yl)methoxy)quinazolin-7-yl)-2-fluoro-6-(methoxymethoxy)naphthalen-1-yl)butanoic acid (45 mg, 61 μmol, 58%).

MS (ESI): m/z (+)=738.4.

### Step F: Synthesis of 2⁸,3⁷-difluoro-2²-(((2R,7aS)-2-fluorotetrahydro-1H-pyrrolizin-7a(5H)-yl)methoxy)-3³-hydroxy-8-aza-1(4,6)-oxazepana-2(4,7)-quinazolina-3(1,8)-naphthalenacyclodecaphan-7-one

A solution of 4-(8-(4-(6-(2-aminoethyl)-1,4-oxazepan-4-yl)-8-fluoro-2-(((2R,7aS)-2-fluorotetrahydro-1H-pyrrolizin-7a(5H)-yl)methoxy)quinazolin-7-yl)-2-fluoro-6-(methoxymethoxy)naphthalen-1-yl)butanoic acid (45 mg, 61 μmol) in DCM (1 mL) was treated with HCl in 1,4-dioxane (0.76 mL, 4 M, 3.0 mmol). The mixture was stirred at room temperature for 30 minutes. The volatiles were removed under a stream of nitrogen, and the residue was further dried under vacuum. The resulting residue was taken up in DMF (5 mL) and treated with HATU (28 mg, 73 μmol) and DIPEA (39 mg, 53 μL, 0.30 mmol). The mixture was stirred at room temperature for 30 minutes. The reaction mixture was purified by reverse-phase flash column chromatography using a gradient of 10-100% B (A: water+0.1% formic acid; B: acetonitrile+0.1% formic acid to afford 2⁸,3⁷-difluoro-2²-(((2R,7aS)-2-fluorotetrahydro-1H-pyrrolizin-7a(5H)-yl)methoxy)-3³-hydroxy-8-aza-1(4,6)-oxazepana-2(4,7)-quinazolina-3(1,8)-naphthalenacyclodecaphan-7-one (15 mg, 22 μmol, 36%).

MS (ESI): m/z (+)=676.5.

¹H NMR (400 MHz, MeOD) δ 7.96-7.88 (m, 1H), 7.65-7.58 (m, 1H), 7.32-7.23 (m, 1H), 7.22-7.16 (m, 2H), 6.89-6.83 (m, 1H), 5.53-5.28 (m, 1H), 4.68-4.59 (m, 1H), 4.56-4.05 (m, 4H), 3.95-3.39 (m, 8H), 3.33-3.24 (m, 2H), 3.12-3.02 (m, 1H), 2.87-1.77 (m, 9H), 1.65-1.08 (m, 6H).

### Example 62. Synthesis of 1⁶-chloro-2⁸-fluoro-2²-(((2R,7aS)-2-fluorotetrahydro-1H-pyrrolizin-7a(5H)-yl)methoxy)-1¹H-6-aza-2(7,4)-pyrido[4,3-d]pyrimidina-1(4,5)-indazola-3(1,3)-piperidinacycloundecaphan-7-one (Compound 196a)

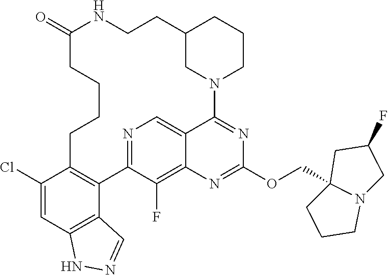 [SMILES: O=C1CCCCc2c(Cl)cc3[nH]ncc3c2-c2ncc3c(nc(OC[C@@]45CCCN4C[C@H](F)C5)nc3c2F)N2CCCC(CCN1)C2]

1⁶-chloro-2⁸-fluoro-2²-(((2R,7aS)-2-fluorotetrahydro-1H-pyrrolizin-7a(5H)-yl)methoxy)-1¹H-6-aza-2(7,4)-pyrido[4,3-d]pyrimidina-1(4,5)-indazola-3(1,3)-piperidinacycloundecaphan-7-one was prepared in an analogous manner to Example 29 by using tert-butyl 5-(6-chloro-1-(tetrahydro-2H-pyran-2-yl)-4-(4,4,5,5-tetramethyl-1,3,2-dioxaborolan-2-yl)-1H-indazol-5-yl)pentanoate in Step A.

MS (ESI): m/z (+)=665.4.

¹H NMR (400 MHz, MeOD) δ 9.13 (s, 1H), 7.71 (s, 1H), 7.63 (s, 1H), 5.47-5.25 (m, 2H), 5.01-4.91 (m, 1H), 4.73-4.65 (m, 1H), 4.52-4.30 (m, 2H), 3.70-2.84 (m, 10H), 2.56-1.13 (m, 18H).

### Example 63. (2⁸,3⁷-difluoro-2²-(((2R,7aS)-2-fluorotetrahydro-1H-pyrrolizin-7a(5H)-yl)methoxy)-3³-hydroxy-8-aza-2(4,7)-pyrido[4,3-d]pyrimidina-1(1,3)-piperidina-3(1,8)-naphthalenacyclodecaphan-7-one) (Compound 118a)

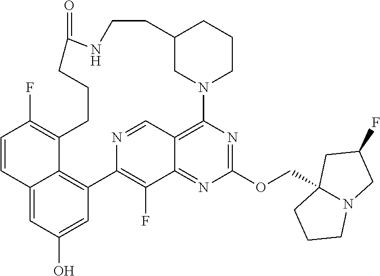 [SMILES: O=C1CCCc2c(F)ccc3cc(O)cc(c23)-c2ncc3c(nc(OC[C@@]45CCCN4C[C@H](F)C5)nc3c2F)N2CCCC(CCN1)C2]

(2⁸,3⁷-difluoro-2²-(((2R,7aS)-2-fluorotetrahydro-1H-pyrrolizin-7a(5H)-yl)methoxy)-3³-hydroxy-8-aza-2(4,7)-pyrido[4,3-d]pyrimidina-1(1,3)-piperidina-3(1,8)-naphthalenacyclodecaphan-7-one) was prepared in an analogous manner to Example 29 by using ethyl 4-(2-fluoro-6-(methoxymethoxy)-8-(4,4,5,5-tetramethyl-1,3,2-dioxaborolan-2-yl)naphthalen-1-yl)butanoate in Step A.

MS (ESI): m/z (+)=661.4.

¹H NMR (400 MHz, MeOD) δ 9.04-8.99 (m, 1H), 7.67-7.58 (m, 1H), 7.26-7.22 (m, 1H), 7.22-7.16 (m, 1H), 7.03-6.98 (m, 1H), 5.50-5.23 (m, 1H), 4.85-4.75 (m, 1H), 4.53-4.34 (m, 3H), 3.70-3.37 (m, 5H), 3.23-3.01 (m, 2H), 2.83-2.61 (m, 2H), 2.55-1.19 (m, 16H), 1.02-0.82 (m, 2H).

### Example 64. (2⁸,3⁷-difluoro-2²-(((2R,7aS)-2-fluorotetrahydro-1H-pyrrolizin-7a(5H)-yl)methoxy)-1³,3³-dihydroxy-8-aza-2(4,7)-pyrido[4,3-d]pyrimidina-1(1,3)-piperidina-3(1,8)-naphthalenacyclodecaphan-7-one) (Compound 136a)

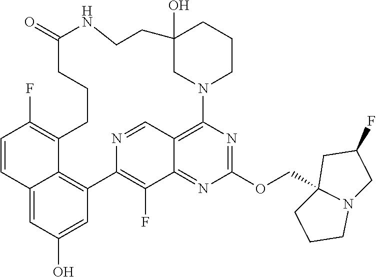 [SMILES: O=C1CCCc2c(F)ccc3cc(O)cc(c23)-c2ncc3c(nc(OC[C@@]45CCCN4C[C@H](F)C5)nc3c2F)N2CCCC(O)(CCN1)C2]

(2⁸,3⁷-difluoro-2²-(((2R,7aS)-2-fluorotetrahydro-1H-pyrrolizin-7a(5H)-yl)methoxy)-1³,3³-dihydroxy-8-aza-2(4,7)-pyrido[4,3-d]pyrimidina-1(1,3)-piperidina-3(1,8)-naphthalenacyclodecaphan-7-one) was prepared in an analogous manner to Example 32 by using ethyl 4-(2-fluoro-6-(methoxymethoxy)-8-(4,4,5,5-tetramethyl-1,3,2-dioxaborolan-2-yl)naphthalen-1-yl)butanoate in Step A.

MS (ESI): m/z (+)=677.4.

¹H NMR (400 MHz, MeOD) δ 9.01-8.96 (m, 1H), 7.67-7.60 (m, 1H), 7.25-7.23 (m, 1H), 7.23-7.16 (m, 1H), 6.99-6.92 (m, 1H), 5.44-5.24 (m, 1H), 4.66-4.04 (m, 4H), 3.86-2.94 (m, 7H), 2.78-2.59 (m, 1H), 2.47-1.21 (m, 18H).

### Example 65. 3³-chloro-2⁸-fluoro-2²-(((2R,7aS)-2-fluorotetrahydro-1H-pyrrolizin-7a(5H)-yl)methoxy)-1³,3⁵-dihydroxy-9-aza-2(4,7)-pyrido[4,3-d]pyrimidina-1(1,3)-piperidina-3(1,2)-benzenacycloundecaphan-8-one—Isomer 1 (Compound 140b)

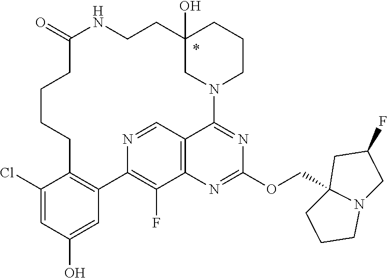 [SMILES: O=C1CCCCc2c(Cl)cc(O)cc2-c2ncc3c(nc(OC[C@@]45CCCN4C[C@H](F)C5)nc3c2F)N2CCCC(O)(CCN1)C2]

The diastereomers of Compound 140a (prepared according to Example 27) were separated using Prep-HPLC with the following conditions: Column: ChiralCel OD-H 21×250 mm; Mobile Phase: 35% Methanol+0.25% diethylamine in CO₂; Flow Rate: 70 mL/min; Sample: 23.2 mg of sample was dissolved in 2 mL methanol+2 mL dichloromethane; Injection: 2.0 mL; Detection: 220 nm. 3³-chloro-2⁸-fluoro-2²-(((2R,7aS)-2-fluorotetrahydro-1H-pyrrolizin-7a(5H)-yl)methoxy)-1³,3⁵-dihydroxy-9-aza-2(4,7)-pyrido[4,3-d]pyrimidina-1(1,3)-piperidina-3(1,2)-benzenacycloundecaphan-8-one (Isomer 1) was provided as the first eluting peak.

LCMS: Retention time=1.85 min, m/z (+)=657.5.

### Example 66. 3³-chloro-2⁸-fluoro-2²-(((2R,7aS)-2-fluorotetrahydro-1H-pyrrolizin-7a(5H)-yl)methoxy)-1³,3⁵-dihydroxy-9-aza-2(4,7)-pyrido[4,3-d]pyrimidina-1(1,3)-piperidina-3(1,2)-benzenacycloundecaphan-8-one—Isomer 2 (Compound 140c)

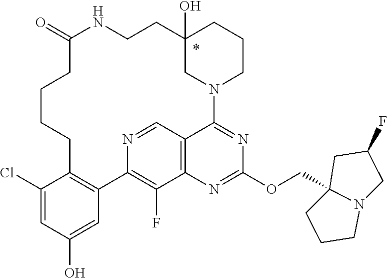 [SMILES: O=C1CCCCc2c(Cl)cc(O)cc2-c2ncc3c(nc(OC[C@@]45CCCN4C[C@H](F)C5)nc3c2F)N2CCCC(O)(CCN1)C2]

The diastereomers of Compound 140a (prepared according to Example 27) were separated using Prep-HPLC with the following conditions: Column: ChiralCel OD-H 21×250 mm; Mobile Phase: 35% Methanol+0.25% diethylamine in CO₂; Flow Rate: 70 mL/min; Sample: 23.2 mg of sample was dissolved in 2 mL methanol+2 mL dichloromethane; Injection: 2.0 mL; Detection: 220 nm. 3³-chloro-2⁸-fluoro-2²-(((2R,7aS)-2-fluorotetrahydro-1H-pyrrolizin-7a(5H)-yl)methoxy)-1³,3⁵-dihydroxy-9-aza-2(4,7)-pyrido[4,3-d]pyrimidina-1(1,3)-piperidina-3(1,2)-benzenacycloundecaphan-8-one (Isomer 2) was provided as the second eluting peak.

LCMS: Retention time=1.87 min, m/z (+)=657.5.

### Example 68. 3⁵-amino-2⁸,3⁴-difluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacycloundecaphan-8-one—Isomer 2 (Compound 159b)

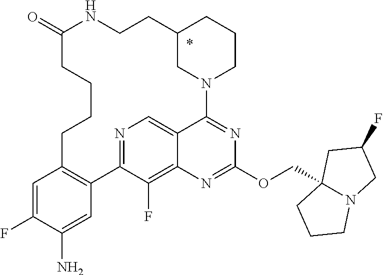 [SMILES: Nc1cc2c(cc1F)CCCCC(=O)NCCC1CCCN(C1)c1nc(OC[C@@]34CCCN3C[C@H](F)C4)nc3c(F)c-2ncc13]

The diastereomers of Compound 159a (prepared according to Example 34) was separated using Prep-HPLC with the following conditions: Column: ChiralCel OJ-3 4.6×50 mm; Mobile Phase: 5% Methanol+0.05% diethylamine in CO₂ to 40% Methanol+0.05% diethylamine in CO₂; Flow Rate: 3 mL/min; Detection: 220 nm. 3⁵-amino-2⁸,3⁴-difluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacycloundecaphan-8-one (Isomer 2) was provided as the second eluting peak.

LCMS: Retention time=1.96 min, m/z (+)=624.1.

### Example 69. 3⁵-amino-2⁸,3⁴-difluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacycloundecaphan-8-one—Isomer 1 (Compound 159c)

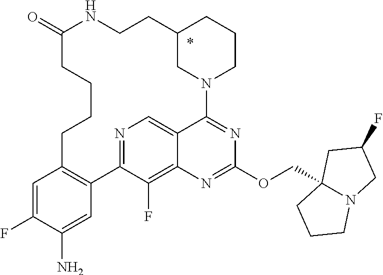 [SMILES: Nc1cc2c(cc1F)CCCCC(=O)NCCC1CCCN(C1)c1nc(OC[C@@]34CCCN3C[C@H](F)C4)nc3c(F)c-2ncc13]

The diastereomers of Compound 159a (prepared according to Example 34) was separated using Prep-HPLC with the following conditions: Column: ChiralCel OJ-3 4.6×50 mm; Mobile Phase: 5% Methanol+0.05% diethylamine in CO₂ to 40% Methanol+0.05% diethylamine in CO₂; Flow Rate: 3 mL/min; Detection: 220 nm. 3⁵-amino-2⁸,3⁴-difluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacycloundecaphan-8-one (Isomer 1) was provided as the first eluting peak.

LCMS: Retention time=1.96 min, m/z (+)=624.1.

### Example 70. (3⁵-amino-3³-chloro-2⁸-fluoro-2²-(((2R,7aS)-2-fluorotetrahydro-1H-pyrrolizin-7a(5H)-yl)methoxy)-9-aza-1(4,6)-oxazepana-2(4,7)-pyrido[4,3-d]pyrimidina-3(1,2)-benzenacyclodecaphan-8-one) (Compound 162a)

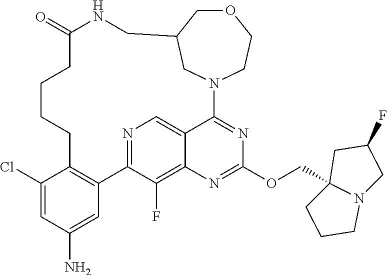 [SMILES: Nc1cc(Cl)c2c(c1)-c1ncc3c(nc(OC[C@@]45CCCN4C[C@H](F)C5)nc3c1F)N1CCOCC(CNC(=O)CCCC2)C1]

(3⁵-amino-3³-chloro-2⁸-fluoro-2²-(((2R,7aS)-2-fluorotetrahydro-1H-pyrrolizin-7a(5H)-yl)methoxy)-9-aza-1(4,6)-oxazepana-2(4,7)-pyrido[4,3-d]pyrimidina-3(1,2)-benzenacyclodecaphan-8-one) was prepared in an analogous manner to Example 29 by using tert-butyl ((4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)methyl)carbamate and methyl 5-(4-(bis(4-methoxybenzyl)amino)-2-chloro-6-(4,4,5,5-tetramethyl-1,3,2-dioxaborolan-2-yl)phenyl)pentanoate in Step A.

MS (ESI): m/z (+)=642.5.

¹H NMR (400 MHz, MeOD) δ 8.97 (s, 1H), 6.81 (d, J=2.4 Hz, 1H), 6.68 (d, J=2.4 Hz, 1H), 5.38-5.18 (m, 1H), 4.35-4.22 (m, 2H), 4.22-4.13 (m, 2H), 4.08-3.82 (m, 6H), 3.43-3.25 (m, 5H), 3.13-2.99 (m, 2H), 2.52-1.78 (m, 10H), 1.49-1.25 (m, 2H), 1.18-1.04 (m, 2H).

### Example 71. (3⁵-amino-2⁸,3⁴-difluoro-2²-(((2R,7aS)-2-fluorotetrahydro-1H-pyrrolizin-7a(5H)-yl)methoxy)-9-aza-2(4,7)-quinazolina-1(1,3)-piperidina-3(1,2)-benzenacycloundecaphan-8-one) (Compound 163a)

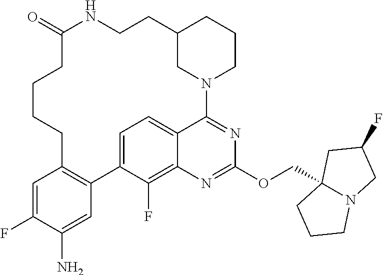 [SMILES: Nc1cc2c(cc1F)CCCCC(=O)NCCC1CCCN(C1)c1nc(OC[C@@]34CCCN3C[C@H](F)C4)nc3c(F)c-2ccc13]

(3⁵-amino-2⁸,3⁴-difluoro-2²-(((2R,7aS)-2-fluorotetrahydro-1H-pyrrolizin-7a(5H)-yl)methoxy)-9-aza-2(4,7)-quinazolina-1(1,3)-piperidina-3(1,2)-benzenacycloundecaphan-8-one) was prepared in an analogous manner to Example 41 by using methyl 5-(4-amino-5-fluoro-2-(4,4,5,5-tetramethyl-1,3,2-dioxaborolan-2-yl)phenyl)pentanoate in Step C.

MS (ESI): m/z (+)=623.4.

¹H NMR (400 MHz, MeOD) δ 7.74-7.63 (m, 1H), 7.16-7.09 (m, 1H), 6.89 (d, J=12.1 Hz, 1H), 6.70 (d, J=9.0 Hz, 1H), 5.57-5.33 (m, 1H), 4.62-4.42 (m, 3H), 3.89-3.63 (m, 2H), 3.39-3.05 (m, 6H), 2.63-1.00 (m, 22H).

### Example 72. (3⁵-amino-3³-chloro-2⁸-fluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-pyrrolidina-3(1,2)-benzenacycloundecaphan-8-one) (Compound 164a)

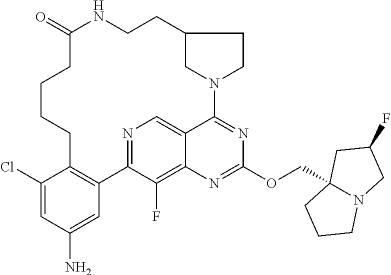 [SMILES: Nc1cc(Cl)c2c(c1)-c1ncc3c(nc(OC[C@@]45CCCN4C[C@H](F)C5)nc3c1F)N1CCC(CCNC(=O)CCCC2)C1]

(3⁵-amino-3³-chloro-2⁸-fluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-pyrrolidina-3(1,2)-benzenacycloundecaphan-8-one) was prepared in an analogous manner to Example 40 by using tert-butyl (2-(pyrrolidin-3-yl)ethyl)carbamate in Step A and methyl 5-(4-(bis(4-methoxybenzyl)amino)-2-chloro-6-(4,4,5,5-tetramethyl-1,3,2-dioxaborolan-2-yl)phenyl)pentanoate in Step C.

MS (ESI): m/z (+)=626.4.

¹H NMR (400 MHz, MeOD) δ 9.04 (s, 1H), 6.78 (d, J=2.3 Hz, 1H), 6.70 (d, J=2.3 Hz, 1H), 5.45-5.27 (m, 1H), 4.52-4.26 (m, 3H), 4.04-3.98 (m, 1H), 3.89-3.79 (m, 2H), 3.75-3.31 (m, 6H), 3.23-3.05 (m, 2H), 2.54-0.82 (m, 17H).

### Example 73. (3⁵-amino-3³-chloro-2⁸-fluoro-2²-(((2R,7aS)-2-fluorotetrahydro-1H-pyrrolizin-7a(5H)-yl)methoxy)-9-aza-2(4,7)-quinazolina-1(1,3)-piperidina-3(1,2)-benzenacycloundecaphan-8-one) (Compound 165a)

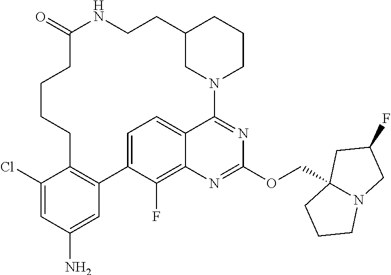 [SMILES: Nc1cc(Cl)c2c(c1)-c1ccc3c(nc(OC[C@@]45CCCN4C[C@H](F)C5)nc3c1F)N1CCCC(CCNC(=O)CCCC2)C1]

(3⁵-amino-3³-chloro-2⁸-fluoro-2²-(((2R,7aS)-2-fluorotetrahydro-1H-pyrrolizin-7a(5H)-yl)methoxy)-9-aza-2(4,7)-quinazolina-1(1,3)-piperidina-3(1,2)-benzenacycloundecaphan-8-one) was prepared in an analogous manner to Example 41 by using methyl 5-(4-(bis(4-methoxybenzyl)amino)-2-chloro-6-(4,4,5,5-tetramethyl-1,3,2-dioxaborolan-2-yl)phenyl)pentanoate in Step C.

MS (ESI): m/z (+)=639.4.

¹H NMR (400 MHz, MeOD) δ 7.76-7.67 (m, 1H), 7.12-7.04 (m, 1H), 6.79-6.75 (m, 1H), 6.52-6.47 (m, 1H), 5.42-5.17 (m, 1H), 4.80-4.72 (m, 1H), 4.60-4.52 (m, 1H), 4.38-4.17 (m, 3H), 3.46-3.15 (m, 4H), 3.11-2.93 (m, 2H), 2.88-2.66 (m, 2H), 2.42-0.98 (m, 20H).

### Example 74. (2⁸-fluoro-2²-(((2R,7aS)-2-fluorotetrahydro-1H-pyrrolizin-7a(5H)-yl)methoxy)-1¹H-6-aza-2(7,4)-quinazolina-1(4,5)-indazola-3(1,3)-piperidinacycloundecaphan-7-one) (Compound 166a)

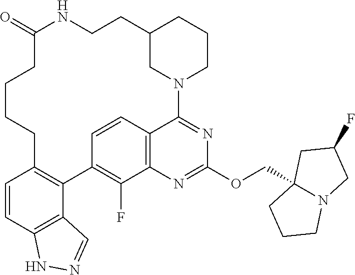 [SMILES: O=C1CCCCc2ccc3[nH]ncc3c2-c2ccc3c(nc(OC[C@@]45CCCN4C[C@H](F)C5)nc3c2F)N2CCCC(CCN1)C2]

(2⁸-fluoro-2²-(((2R,7aS)-2-fluorotetrahydro-1H-pyrrolizin-7a(5H)-yl)methoxy)-1¹H-6-aza-2(7,4)-quinazolina-1(4,5)-indazola-3(1,3)-piperidinacycloundecaphan-7-one) was prepared in an analogous manner to Example 41 by using ethyl 5-(1-(tetrahydro-2H-pyran-2-yl)-4-(4,4,5,5-tetramethyl-1,3,2-dioxaborolan-2-yl)-1H-indazol-5-yl)pentanoate in Step C.

MS (ESI): m/z (+)=630.4.

¹H NMR (400 MHz, MeOD) δ 7.82-7.76 (m, 1H), 7.60-7.57 (m, 1H), 7.53-7.50 (m, 1H), 7.37-7.32 (m, 1H), 7.26-7.18 (m, 1H), 5.38-5.20 (m, 1H), 4.61-4.54 (m, 1H), 4.39-4.08 (m, 2H), 3.44-1.05 (m, 30H).

### Example 75. (2⁸-fluoro-2²-(((2R,7aS)-2-fluorotetrahydro-1H-pyrrolizin-7a(5H)-yl)methoxy)-1¹H-6-aza-2(7,4)-pyrido[4,3-d]pyrimidina-1(4,5)-indazola-3(1,3)-piperidinacycloundecaphan-7-one) (Compound 167a)

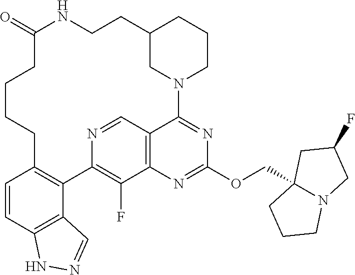 [SMILES: O=C1CCCCc2ccc3[nH]ncc3c2-c2ncc3c(nc(OC[C@@]45CCCN4C[C@H](F)C5)nc3c2F)N2CCCC(CCN1)C2]

(2⁸-fluoro-2²-(((2R,7aS)-2-fluorotetrahydro-1H-pyrrolizin-7a(5H)-yl)methoxy)-11H-6-aza-2(7,4)-pyrido[4,3-d]pyrimidina-1(4,5)-indazola-3(1,3)-piperidinacycloundecaphan-7-one) was prepared in an analogous manner to Example 29 by using ethyl 5-(1-(tetrahydro-2H-pyran-2-yl)-4-(4,4,5,5-tetramethyl-1,3,2-dioxaborolan-2-yl)-1H-indazol-5-yl)pentanoate in Step A.

MS (ESI): m/z (+)=631.4.

¹H NMR (400 MHz, MeOD) δ 9.12 (s, 1H), 7.66 (s, 1H), 7.56 (d, J=8.6 Hz, 1H), 7.36 (d, J=8.6 Hz, 1H), 5.53-5.31 (m, 1H), 5.00-4.88 (m, 1H), 4.71-4.60 (m, 1H), 4.58-4.33 (m, 2H), 3.76-3.51 (m, 4H), 3.42-3.02 (m, 4H), 2.99-2.85 (m, 1H), 2.73-1.09 (m, 20H).

### Example 76. (1⁶-amino-1²-chloro-2⁶,2⁸-difluoro-2²-(((2R,7aS)-2-fluorotetrahydro-1H-pyrrolizin-7a(5H)-yl)methoxy)-3³-hydroxy-6-aza-2(7,4)-quinazolina-1(4,3)-pyridina-3(1,3)-piperidinacycloundecaphan-7-one)—Atropisomer 1 (Compound 188a)

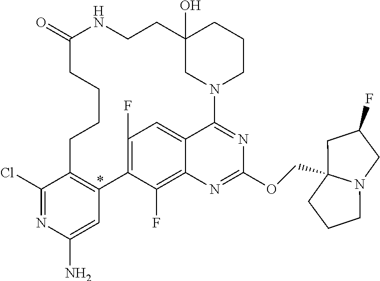 [SMILES: Nc1cc2c(c(Cl)n1)CCCCC(=O)NCCC1(O)CCCN(C1)c1nc(OC[C@@]34CCCN3C[C@H](F)C4)nc3c(F)c-2c(F)cc13]

(1⁶-amino-1²-chloro-2⁶,2⁸-difluoro-2²-(((2R,7aS)-2-fluorotetrahydro-1H-pyrrolizin-7a(5H)-yl)methoxy)-3³-hydroxy-6-aza-2(7,4)-quinazolina-1(4,3)-pyridina-3(1,3)-piperidinacycloundecaphan-7-one) (atropisomer 1) was prepared in an analogous manner to Example 50 by using methyl 5-(6-(bis(4-methoxybenzyl)amino)-2-chloro-4-(4,4,5,5-tetramethyl-1,3,2-dioxaborolan-2-yl)pyridin-3-yl)pentanoate in Step C as the second eluting peak after HPLC purification (Retention time=8.1 min, Method A).

MS (ESI): m/z (+)=674.4.

¹H NMR (400 MHz, MeOD) δ 8.03 (d, J=9.7 Hz, 1H), 6.41 (s, 1H), 5.42-5.19 (m, 1H), 4.36-4.29 (m, 1H), 4.29-4.22 (m, 1H), 4.22-4.07 (m, 2H), 3.88-3.75 (m, 1H), 3.45-3.16 (m, 3H), 3.12-3.02 (m, 3H), 2.97-2.89 (m, 1H), 2.58-2.47 (m, 1H), 2.45-1.00 (m, 19H).

### Example 77. (1⁶-amino-1²-chloro-2⁶,2⁸-difluoro-2²-(((2R,7aS)-2-fluorotetrahydro-1H-pyrrolizin-7a(5H)-yl)methoxy)-3³-hydroxy-6-aza-2(7,4)-quinazolina-1(4,3)-pyridina-3(1,3)-piperidinacycloundecaphan-7-one)—Atropisomer 2 (Compound 188b)

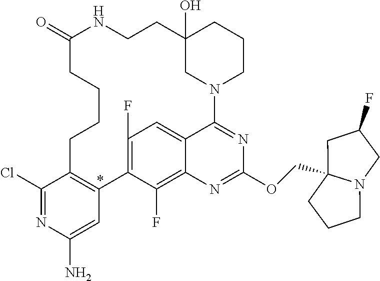 [SMILES: Nc1cc2c(c(Cl)n1)CCCCC(=O)NCCC1(O)CCCN(C1)c1nc(OC[C@@]34CCCN3C[C@H](F)C4)nc3c(F)c-2c(F)cc13]

(1⁶-amino-1²-chloro-2⁶,2⁸-difluoro-2²-(((2R,7aS)-2-fluorotetrahydro-1H-pyrrolizin-7a(5H)-yl)methoxy)-3³-hydroxy-6-aza-2(7,4)-quinazolina-1(4,3)-pyridina-3(1,3)-piperidinacycloundecaphan-7-one) (atropisomer 2) was prepared in an analogous manner to Example 50 by using methyl 5-(6-(bis(4-methoxybenzyl)amino)-2-chloro-4-(4,4,5,5-tetramethyl-1,3,2-dioxaborolan-2-yl)pyridin-3-yl)pentanoate in Step C as the first eluting peak after HPLC purification (Retention time=7.8 min, Method A).

MS (ESI): m/z (+)=674.4.

¹H NMR (400 MHz, MeOD) δ 7.59 (d, J=9.8 Hz, 1H), 6.37 (s, 1H), 5.42-5.13 (m, 1H), 4.69-4.60 (m, 1H), 4.53-4.39 (m, 1H), 4.42-4.20 (m, 2H), 3.58-3.51 (m, 1H), 3.48-3.16 (m, 4H), 3.14-3.03 (m, 2H), 2.61-2.49 (m, 1H), 2.48-1.13 (m, 20H).

### Example 78. 1⁶-chloro-2²-((1-((dimethylamino)methyl)cyclopropyl)methoxy)-2⁸-fluoro-1¹H-6-aza-2(7,4)-quinazolina-1(4,5)-indazola-3(1,3)-piperidinacycloundecaphan-7-one—Isomer 1 (Compound 197a)

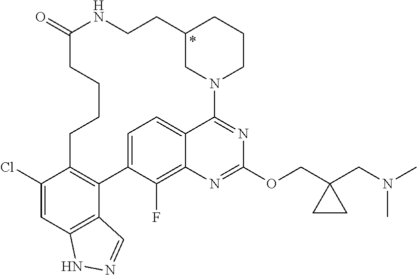 [SMILES: CN(C)CC1(COc2nc3c4ccc(c(F)c4n2)-c2c(c(Cl)cc4[nH]ncc24)CCCCC(=O)NCCC2CCCN3C2)CC1]

1⁶-chloro-2²-((1-((dimethylamino)methyl)cyclopropyl)methoxy)-2⁸-fluoro-1¹H-6-aza-2(7,4)-quinazolina-1(4,5)-indazola-3(1,3)-piperidinacycloundecaphan-7-one (Isomer 1) was prepared in an analogous manner to Example 41 by using (1-((dimethylamino)methyl)cyclopropyl)methanol in Step B and tert-butyl 5-(6-chloro-1-(tetrahydro-2H-pyran-2-yl)-4-(4,4,5,5-tetramethyl-1,3,2-dioxaborolan-2-yl)-1H-indazol-5-yl)pentanoate in Step C as the first eluting peak after HPLC purification (Retention time=10.2 min, Method A).

LCMS: Retention time=2.14 min, m/z (+)=634.4.

¹H NMR (400 MHz, MeOD) δ 7.86 (d, J=8.6 Hz, 1H), 7.69 (s, 1H), 7.55 (s, 1H), 7.27-7.20 (m, 1H), 4.67-4.56 (m, 1H), 4.43-4.25 (m, 2H), 3.44-3.20 (m, 2H), 3.12-2.82 (m, 3H), 2.81-2.66 (m, 7H), 2.57-2.46 (m, 1H), 2.04-0.66 (m, 19H).

### Example 79. 1⁶-chloro-2²-((1-((dimethylamino)methyl)cyclopropyl)methoxy)-2⁸-fluoro-1¹H-6-aza-2(7,4)-quinazolina-1(4,5)-indazola-3(1,3)-piperidinacycloundecaphan-7-one—Isomer 2 (Compound 197b)

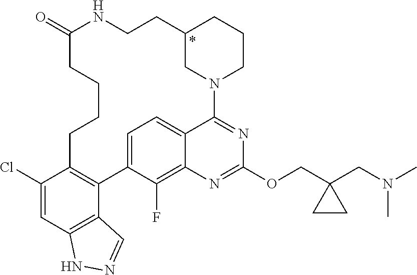 [SMILES: CN(C)CC1(COc2nc3c4ccc(c(F)c4n2)-c2c(c(Cl)cc4[nH]ncc24)CCCCC(=O)NCCC2CCCN3C2)CC1]

1⁶-chloro-2²-((1-((dimethylamino)methyl)cyclopropyl)methoxy)-2⁸-fluoro-1¹H-6-aza-2(7,4)-quinazolina-1(4,5)-indazola-3(1,3)-piperidinacycloundecaphan-7-one (Isomer 2) was prepared in an analogous manner to Example 41 by using (1-((dimethylamino)methyl)cyclopropyl)methanol in Step B and tert-butyl 5-(6-chloro-1-(tetrahydro-2H-pyran-2-yl)-4-(4,4,5,5-tetramethyl-1,3,2-dioxaborolan-2-yl)-1H-indazol-5-yl)pentanoate in Step C as the second eluting peak after HPLC purification (Retention time=11.0 min, Method A).

LCMS: Retention time=2.17 min, m/z (+)=634.5.

### Example 80. (3⁶-chloro-2⁸-fluoro-2²-(((2R,7aS)-2-fluorotetrahydro-1H-pyrrolizin-7a(5H)-yl)methoxy)-3¹H-9-aza-1(4,6)-oxazepana-2(4,7)-quinazolina-3(4,5)-indazolacyclodecaphan-8-one) (Compound 198a)

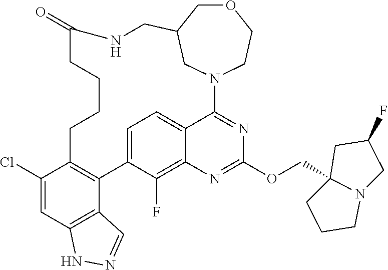 [SMILES: O=C1CCCCc2c(Cl)cc3[nH]ncc3c2-c2ccc3c(nc(OC[C@@]45CCCN4C[C@H](F)C5)nc3c2F)N2CCOCC(CN1)C2]

(3⁶-chloro-2⁸-fluoro-2²-(((2R,7aS)-2-fluorotetrahydro-1H-pyrrolizin-7a(5H)-yl)methoxy)-3¹H-9-aza-1(4,6)-oxazepana-2(4,7)-quinazolina-3(4,5)-indazolacyclodecaphan-8-one) was prepared in an analogous manner to Example 41 by using tert-butyl ((1,4-oxazepan-6-yl)methyl)carbamate in Step A and tert-butyl 5-(6-chloro-1-(tetrahydro-2H-pyran-2-yl)-4-(4,4,5,5-tetramethyl-1,3,2-dioxaborolan-2-yl)-1H-indazol-5-yl)pentanoate in Step C.

MS (ESI): m/z (+)=666.3.

¹H NMR (400 MHz, MeOD) δ 7.90-7.80 (m, 1H), 7.71-7.66 (m, 1H), 7.58-7.54 (m, 1H), 7.26-7.19 (m, 1H), 5.55-5.30 (m, 1H), 4.59-4.40 (m, 3H), 4.35-4.09 (m, 2H), 4.05-3.56 (m, 7H), 3.44-3.26 (m, 3H), 2.98-2.78 (m, 2H), 2.65-1.08 (m, 14H).

### Example 81. (1⁶-chloro-2⁸-fluoro-2²-(((2R,7aS)-2-fluorotetrahydro-1H-pyrrolizin-7a(5H)-yl)methoxy)-3³-hydroxy-1¹H-6-aza-2(7,4)-quinazolina-1(4,5)-indazola-3(1,3)-piperidinacycloundecaphan-7-one) (Compound 199a)

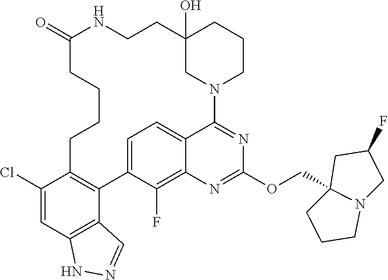 [SMILES: O=C1CCCCc2c(Cl)cc3[nH]ncc3c2-c2ccc3c(nc(OC[C@@]45CCCN4C[C@H](F)C5)nc3c2F)N2CCCC(O)(CCN1)C2]

(1⁶-chloro-2⁸-fluoro-2²-(((2R,7aS)-2-fluorotetrahydro-1H-pyrrolizin-7a(5H)-yl)methoxy)-3³-hydroxy-1¹H-6-aza-2(7,4)-quinazolina-1(4,5)-indazola-3(1,3)-piperidinacycloundecaphan-7-one) was prepared in an analogous manner to Example 41 by using tert-butyl (2-(3-hydroxypiperidin-3-yl)ethyl)carbamate in Step A and tert-butyl 5-(6-chloro-1-(tetrahydro-2H-pyran-2-yl)-4-(4,4,5,5-tetramethyl-1,3,2-dioxaborolan-2-yl)-1H-indazol-5-yl)pentanoate in Step C.

MS (ESI): m/z (+)=680.4.

¹H NMR (400 MHz, MeOD) δ 7.89-7.83 (m, 1H), 7.71-7.66 (m, 1H), 7.53-7.48 (m, 1H), 7.26-7.18 (m, 1H), 5.56-5.33 (m, 1H), 4.80-4.71 (m, 1H), 4.68-4.31 (m, 3H), 4.03-3.56 (m, 4H), 3.47-3.05 (m, 4H), 2.94-1.11 (m, 20H).

### Example 82. 3⁶-chloro-2⁶,2⁸-difluoro-2²-(((2R,7aS)-2-fluorotetrahydro-1H-pyrrolizin-7a(5H)-yl)methoxy)-3¹H-9-aza-1(4,6)-oxazepana-2(4,7)-quinazolina-3(4,5)-indazolacyclodecaphan-8-one (Compound 200a)

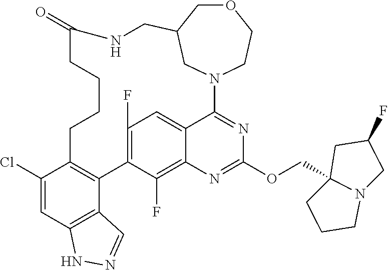 [SMILES: O=C1CCCCc2c(Cl)cc3[nH]ncc3c2-c2c(F)cc3c(nc(OC[C@@]45CCCN4C[C@H](F)C5)nc3c2F)N2CCOCC(CN1)C2]

3⁶-chloro-2⁶,2⁸-difluoro-2²-(((2R,7aS)-2-fluorotetrahydro-1H-pyrrolizin-7a(5H)-yl)methoxy)-3¹H-9-aza-1(4,6)-oxazepana-2(4,7)-quinazolina-3(4,5)-indazolacyclodecaphan-8-one was prepared in an analogous manner to Example 50 by using tert-butyl ((1,4-oxazepan-6-yl)methyl)carbamate in Step A and tert-butyl 5-(6-chloro-1-(tetrahydro-2H-pyran-2-yl)-4-(4,4,5,5-tetramethyl-1,3,2-dioxaborolan-2-yl)-1H-indazol-5-yl)pentanoate in Step C.

MS (ESI): m/z (+)=684.3.

¹H NMR (400 MHz, MeOD) δ 7.77-7.63 (m, 2H), 7.62-7.56 (m, 1H), 5.48-5.27 (m, 1H), 4.49-4.07 (m, 3H), 4.03-3.28 (m, 8H), 3.24-3.07 (m, 2H), 2.96-2.77 (m, 2H), 2.61-1.14 (m, 16H).

### Example 83. (1³R,1⁵R)-3⁵-amino-3³-chloro-1⁵,2⁸-difluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacyclononaphan-8-one (Compound 201a)

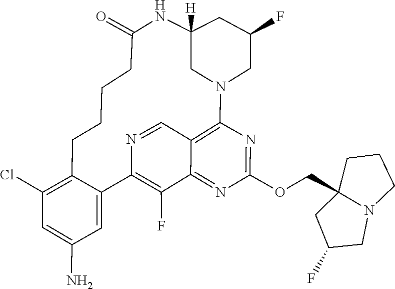 [SMILES: Nc1cc(Cl)c2c(c1)-c1ncc3c(nc(OC[C@@]45CCCN4C[C@H](F)C5)nc3c1F)N1C[C@H](F)C[C@H](C1)NC(=O)CCCC2]

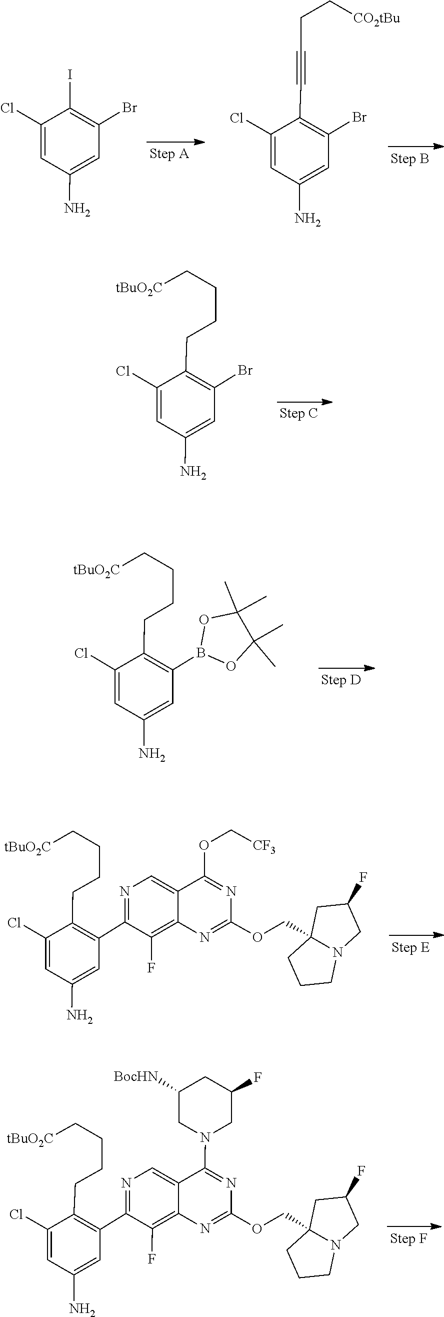 [SMILES: Nc1cc(Cl)c(I)c(Br)c1 ; CC(C)(C)OC(=O)CCC#Cc1c(Cl)cc(N)cc1Br ; CC(C)(C)OC(=O)CCCCc1c(Cl)cc(N)cc1Br ; CC(C)(C)OC(=O)CCCCc1c(Cl)cc(N)cc1B1OC(C)(C)C(C)(C)O1 ; CC(C)(C)OC(=O)CCCCc1c(Cl)cc(N)cc1-c1ncc2c(OCC(F)(F)F)nc(OC[C@@]34CCCN3C[C@H](F)C4)nc2c1F ; CC(C)(C)OC(=O)CCCCc1c(Cl)cc(N)cc1-c1ncc2c(N3C[C@H](F)C[C@@H](NC(=O)OC(C)(C)C)C3)nc(OC[C@@]34CCCN3C[C@H](F)C4)nc2c1F]

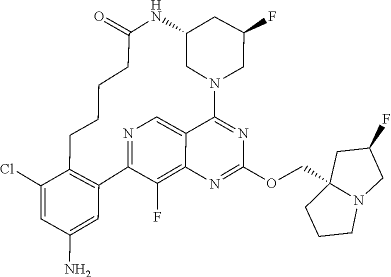 [SMILES: Nc1cc(Cl)c2c(c1)-c1ncc3c(nc(OC[C@@]45CCCN4C[C@H](F)C5)nc3c1F)N1C[C@H](F)C[C@H](C1)NC(=O)CCCC2]

### Step A: Synthesis of tert-butyl 5-(4-amino-2-bromo-6-chlorophenyl)pent-4-ynoate

A solution of 3-bromo-5-chloro-4-iodoaniline (21 g, 63 mmol), tert-butyl pent-4-ynoate (24 g, 0.16 μmol), PdCl₂PPh₃ (13 g, 19 mmol), and CuI (3.6 g, 19 mmol) in a mixture of Et₃N and DCM (1:1, 250 mL) was degassed and purged with N₂. The resulting mixture was warmed at 60° C. for 3 hours. The reaction mixture was cooled to room temperature, diluted with water and EtOAc, washed with brine, dried (Na₂SO₄), filtered, and concentrated. The resulting residue was purified by flash column chromatography (ethyl acetate/petroleum ether) to afford tert-butyl 5-(4-amino-2-bromo-6-chlorophenyl)pent-4-ynoate (16 g, 45 mmol, 71%) as a yellow solid.

MS (ESI): m/z (+)=357.9.

### Step B: Synthesis of tert-butyl 5-(4-amino-2-bromo-6-chlorophenyl)pentanoate

A solution of tert-butyl 5-(4-amino-2-bromo-6-chlorophenyl)pent-4-ynoate (200 mg, 558 μmol) in MeOH (1.5 mL) and EtOAc (1.5 mL) was treated with PtO₂ (25.3 mg, 112 μmol). The reaction mixture was stirred under a hydrogen atmosphere at room temperature for 4 hours. The reaction mixture was filtered through celite, concentrated, and purified by prep-TLC to afford tert-butyl 5-(4-amino-2-bromo-6-chlorophenyl)pentanoate (170 mg, 469 μmol, 84.1%) as a yellow oil.

MS (ESI): m/z (+)=305.9 [M+H-tBu].

### Step C: Synthesis of tert-butyl 5-(4-amino-2-chloro-6-(4,4,5,5-tetramethyl-1,3,2-dioxaborolan-2-yl)phenyl)pentanoate

A solution of tert-butyl 5-(4-amino-2-bromo-6-chlorophenyl)pentanoate (3500 mg, 9.65 mmol), 4,4,4′,4′,5,5,5′,5′-octamethyl-2,2′-bi(1,3,2-dioxaborolane) (4.90 g, 19.3 mmol), potassium acetate (2.84 g, 1.81 mL, 29.0 mmol) and Pd(dppf)Cl₂·CH₂Cl₂ (788 mg, 965 μmol) in 1,4-dioxane (35 mL) was degassed and purged with N₂ and warmed at 80° C. for 12 hour. The reaction mixture was diluted with EtOAc and water, washed with brine, dried (Na₂SO₄), filtered, and concentrated. The resulting residue was purified by flash column chromatography (ethyl acetate/petroleum ether) to afford tert-butyl 5-(4-amino-2-chloro-6-(4,4,5,5-tetramethyl-1,3,2-dioxaborolan-2-yl)phenyl)pentanoate (3.5 g, 7.7 mmol, 80%) as a yellow solid.

MS (ESI): m/z (+)=410.1.

### Step D: Synthesis of tert-butyl 5-(4-amino-2-chloro-6-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)phenyl)pentanoate

A solution of tert-butyl 5-(4-amino-2-chloro-6-(4,4,5,5-tetramethyl-1,3,2-dioxaborolan-2-yl)phenyl)pentanoate (500 mg, 1.22 mmol), 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (642 mg, 1.46 mmol), potassium phosphate, tribasic (777 mg, 2.44 mL, 1.5 M, 3.66 mmol) and PdCl₂(dtbpf) (79.5 mg, 122 μmol) in 1,4-dioxane (5 mL) was degassed, purged with N₂, and warmed at 40° C. for 12 hours. The reaction mixture was diluted with EtOAc and water, washed with brine, dried (Na₂SO₄), filtered, and concentrated. The resulting residue was purified by flash column chromatography (ethyl acetate/petroleum ether) to afford tert-butyl 5-(4-amino-2-chloro-6-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)phenyl)pentanoate (250 mg, 364 μmol, 29.9%) as a yellow solid.

MS (ESI): m/z (+)=686.1.

### Step E: Synthesis of tert-butyl 5-(4-amino-2-(4-((3R,5R)-3-((tert-butoxycarbonyl)amino)-5-fluoropiperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chlorophenyl)pentanoate

A solution of tert-butyl 5-(4-amino-2-chloro-6-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)phenyl)pentanoate (150 mg, 219 μmol) and tert-butyl ((3R,5R)-5-fluoropiperidin-3-yl)carbamate (57.3 mg, 262 μmol) in DMSO (1.5 mL) was treated with K₂CO₃ (90.6 mg, 656 μmol). The mixture was stirred at 40° C. for 2 hours. The reaction mixture was diluted with EtOAc and water, washed with brine, dried (Na₂SO₄), filtered, and concentrated. The resulting residue was purified by prep-TLC (10:1 DCM:MeOH) to afford tert-butyl 5-(4-amino-2-(4-((3R,5R)-3-((tert-butoxycarbonyl)amino)-5-fluoropiperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chlorophenyl)pentanoate (100 mg, 124 μmol, 56.9%) as a yellow solid.

MS (ESI): m/z (+)=804.2.

### Step F: Synthesis of (1³R,1⁵R)-3⁵-amino-3³-chloro-1⁵,2⁸-difluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacyclononaphan-8-one

A solution of tert-butyl 5-(4-amino-2-(4-((3R,5R)-3-((tert-butoxycarbonyl)amino)-5-fluoropiperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-chlorophenyl)pentanoate (30 mg, 37 μmol) in DCM (0.1 mL) at room temperature was treated with HCl in 1,4-dioxane (0.2 g, 0.2 mL, 4 M, 0.8 mmol). The reaction mixture was stirred for 30 minutes. The reaction mixture was concentrated and dried under vacuum to provide a residue. The residue was taken up in MeCN (1.8 mL) and treated with 1-methyl-1H-imidazole (27 mg, 0.32 mmol) and N-(chloro(dimethylamino)methylene)-N-methylmethanaminium hexafluorophosphate(V) (22 mg, 79 μmol). The reaction mixture was stirred at room temperature for 12 hours. The reaction mixture was filtered and was purified by HPLC (column: Phenomenex luna C18, 150×25 mm, Mobile phase A: water+0.1% NH₄HCO₃ (v/v), Mobile phase B: MeCN, 25%-55% B over 10 min) to afford (1³R,1⁵R)-3⁵-amino-3³-chloro-1⁵,2⁸-difluoro-2²-(((2R,7aS)-2-fluorotetrahydro-1H-pyrrolizin-7a(5H)-yl)methoxy)-9-aza-2(4,7)-pyrido[4,3-d]pyrimidina-1(1,3)-piperidina-3(1,2)-benzenacyclononaphan-8-one (0.72 mg, 1.0 μmol, 2.3%) as a yellow solid.

MS (ESI): m/z (+)=630.1.

### Example 84. 1⁶-chloro-2⁸-fluoro-2²-(((2R,7aS)-2-fluorotetrahydro-1H-pyrrolizin-7a(5H)-yl)methoxy)-1¹H-6-aza-2(7,4)-quinazolina-1(4,5)-indazola-3(1,3)-piperidinacycloundecaphan-7-one—Isomer 1 (Compound 202a)

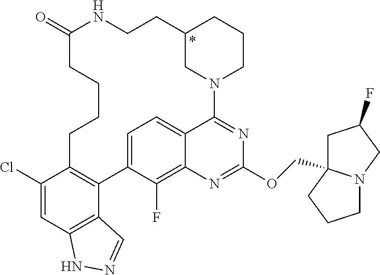 [SMILES: O=C1CCCCc2c(Cl)cc3[nH]ncc3c2-c2ccc3c(nc(OC[C@@]45CCCN4C[C@H](F)C5)nc3c2F)N2CCCC(CCN1)C2]

1⁶-chloro-2⁸-fluoro-2²-(((2R,7aS)-2-fluorotetrahydro-1H-pyrrolizin-7a(5H)-yl)methoxy)-1¹H-6-aza-2(7,4)-quinazolina-1(4,5)-indazola-3(1,3)-piperidinacycloundecaphan-7-one (Isomer 1) was prepared in an analogous manner to Example 41 by using tert-butyl 5-(6-chloro-1-(tetrahydro-2H-pyran-2-yl)-4-(4,4,5,5-tetramethyl-1,3,2-dioxaborolan-2-yl)-1H-indazol-5-yl)pentanoate in Step C as the first eluting peak after HPLC purification (Retention time=10.9 min, Method A).

LCMS: Retention time=2.17 min, m/z (+)=664.5.

¹H NMR (400 MHz, MeOD) δ 7.86-7.80 (m, 1H), 7.67 (s, 1H), 7.56-7.53 (m, 1H), 7.25-7.17 (m, 1H), 5.41-5.20 (m, 1H), 4.83-4.74 (m, 1H), 4.64-4.54 (m, 1H), 4.41-4.22 (m, 2H), 3.52-3.18 (m, 6H), 3.13-2.78 (m, 4H), 2.58-2.47 (m, 1H), 2.43-1.08 (m, 18H).

### Example 85. 1⁶-chloro-2⁸-fluoro-2²-(((2R,7aS)-2-fluorotetrahydro-1H-pyrrolizin-7a(5H)-yl)methoxy)-1¹H-6-aza-2(7,4)-quinazolina-1(4,5)-indazola-3(1,3)-piperidinacycloundecaphan-7-one—Isomer 2 (Compound 202b)

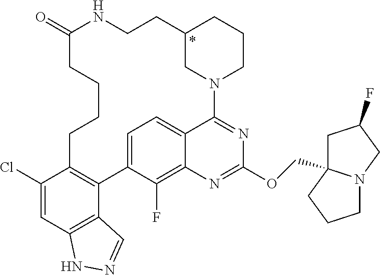 [SMILES: O=C1CCCCc2c(Cl)cc3[nH]ncc3c2-c2ccc3c(nc(OC[C@@]45CCCN4C[C@H](F)C5)nc3c2F)N2CCCC(CCN1)C2]

1⁶-Chloro-2⁸-fluoro-2²-(((2R,7aS)-2-fluorotetrahydro-1H-pyrrolizin-7a(5H)-yl)methoxy)-1¹H-6-aza-2(7,4)-quinazolina-1(4,5)-indazola-3(1,3)-piperidinacycloundecaphan-7-one (Isomer 2) was prepared in an analogous manner to Example 41 by using tert-butyl 5-(6-chloro-1-(tetrahydro-2H-pyran-2-yl)-4-(4,4,5,5-tetramethyl-1,3,2-dioxaborolan-2-yl)-1H-indazol-5-yl)pentanoate in Step C as the second eluting peak after HPLC purification (Retention time=11.6 min, Method A).

LCMS: Retention time=2.19 min, m/z (+)=664.4.

¹H NMR (400 MHz, MeOD) δ 7.89-7.84 (m, 1H), 7.68 (s, 1H), 7.56-7.48 (m, 1H), 7.29-7.23 (m, 1H), 5.54-5.31 (m, 1H), 4.87-4.66 (m, 1H), 4.65-4.34 (m, 3H), 3.84-3.19 (m, 6H), 3.15-1.09 (m, 23H).

### Example 86. 3⁶-chloro-2⁸-fluoro-2²-(((2R,7aS)-2-fluorotetrahydro-1H-pyrrolizin-7a(5H)-yl)methoxy)-3¹H-9-aza-1(4,6)-oxazepana-2(4,7)-pyrido[4,3-d]pyrimidina-3(4,5)-indazolacyclodecaphan-8-one (Compound 203a)

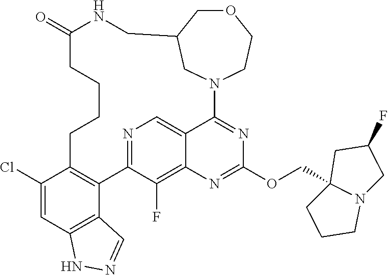 [SMILES: O=C1CCCCc2c(Cl)cc3[nH]ncc3c2-c2ncc3c(nc(OC[C@@]45CCCN4C[C@H](F)C5)nc3c2F)N2CCOCC(CN1)C2]

3⁶-chloro-2⁸-fluoro-2²-(((2R,7aS)-2-fluorotetrahydro-1H-pyrrolizin-7a(5H)-yl)methoxy)-3¹H-9-aza-1(4,6)-oxazepana-2(4,7)-pyrido[4,3-d]pyrimidina-3(4,5)-indazolacyclodecaphan-8-one was prepared in an analogous manner to Example 40 by using tert-butyl 5-(6-chloro-1-(tetrahydro-2H-pyran-2-yl)-4-(4,4,5,5-tetramethyl-1,3,2-dioxaborolan-2-yl)-1H-indazol-5-yl)pentanoate in Step B.

MS (ESI): m/z (+)=667.3.

¹H NMR (400 MHz, MeOD) δ 9.11 (s, 1H), 7.73 (s, 1H), 7.66 (s, 1H), 5.57-5.35 (m, 1H), 4.63-4.28 (m, 4H), 4.19-3.57 (m, 8H), 3.46-3.16 (m, 4H), 3.03-1.11 (m, 15H).

### Example 87. 3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclodecaphane-1³,3⁵-diol (Compound 152a)

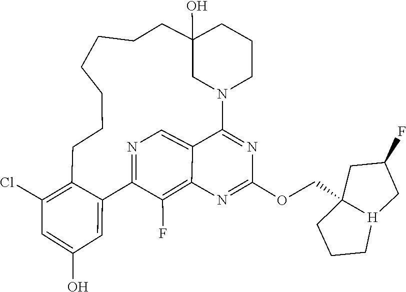

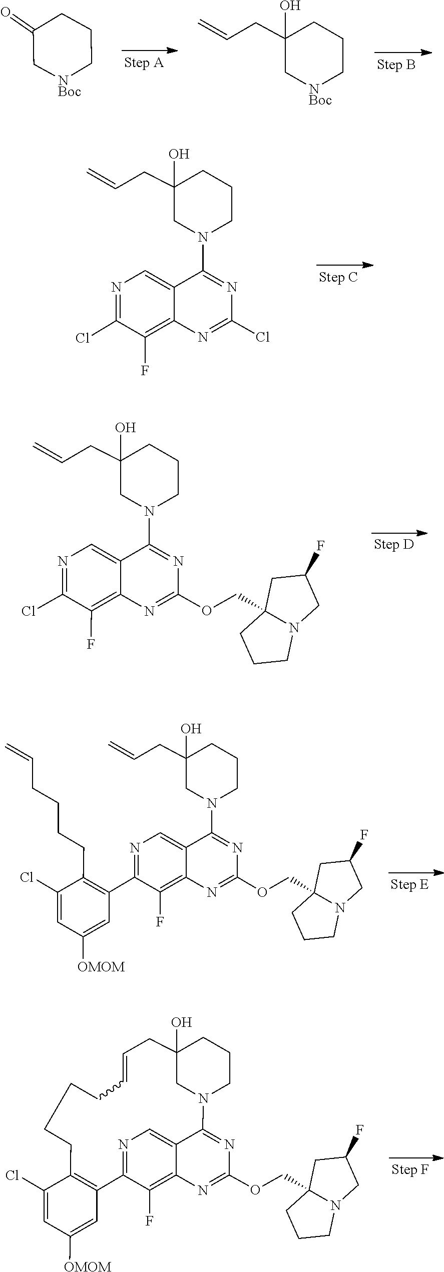

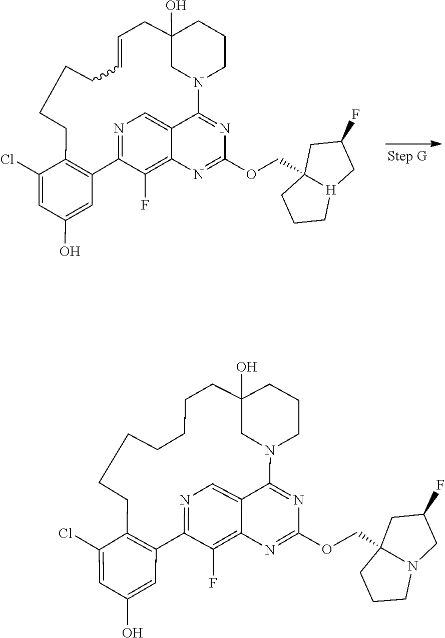

### Step A: Synthesis of tert-butyl 3-allyl-3-hydroxypiperidine-1-carboxylate

A solution of tert-butyl 3-oxopiperidine-1-carboxylate (1.32 g, 6.64 mmol) and molecular sieves in diethyl ether (13 mL) under nitrogen was cooled to −78° C. and allylmagnesium bromide (965 mg, 6.64 mL, 1.0 M, 6.64 mmol) was added dropwise. The reaction mixture was stirred at −78° C. for 3 hours. The reaction mixture was quenched with saturated aqueous NH₄Cl and diluted with water and diethyl ether, washed with brine, dried (MgSO₄), filtered, and concentrated to afford a residue. The residue was taken up in EtOAc and washed with saturated aqueous NH₄Cl, dried (MgSO₄), filtered, and concentrated to afford tert-butyl 3-allyl-3-hydroxypiperidine-1-carboxylate (1.13 g, 4.69 mmol, 71%) as a yellow oil.

¹H NMR (400 MHz, CDCl₃) δ 5.93-5.80 (m, 1H), 5.19-5.04 (m, 2H), 3.80-3.27 (m, 2H), 3.27-2.89 (m, 2H), 2.32-2.12 (m, 2H), 1.79-1.55 (m, 2H), 1.54-1.32 (m, 11H).

### Step B: Synthesis of 3-allyl-1-(2,7-dichloro-8-fluoropyrido[4,3-d]pyrimidin-4-yl)piperidin-3-ol

A solution of tert-butyl 3-allyl-3-hydroxypiperidine-1-carboxylate (177 mg, 733 μmol) in DCM (5 mL) at room temperature was treated with TFA (836 mg, 565 μL, 7.33 mmol). The mixture was stirred for 15 minutes. The reaction mixture was concentrated and dried under vacuum. The resulting residue was taken up in DCM (2 mL) and added dropwise into a solution of 2,4,7-trichloro-8-fluoropyrido[4,3-d]pyrimidine (150.0 mg, 594.2 μmol) and DIPEA (1.54 g, 2.07 mL, 11.9 mmol) in DCM (6 mL) under nitrogen at 0° C. The reaction mixture was stirred for 1 hour. The reaction mixture was concentrated and was purified by flash column chromatography (ethyl acetate/heptane) to afford 3-allyl-1-(2,7-dichloro-8-fluoropyrido[4,3-d]pyrimidin-4-yl)piperidin-3-ol (208 mg, 582 μmol, 98.0%).

MS (ESI): m/z (+)=357.0.

### Step C: Synthesis of 3-allyl-1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-ol

A solution of ((2R,7aS)-2-fluorotetrahydro-1H-pyrrolizin-7a(5H)-yl)methanol (111 mg, 699 μmol) in DCM (12 mL) at −78° C. was treated with LHMDS in THF (117 mg, 699 μL, 1.0 M, 699 μmol). The mixture was stirred for 30 minutes at −78° C. and was then allowed to warm to room temperature. The mixture was stirred for 10 minutes. This solution was then added dropwise into a solution of 3-allyl-1-(2,7-dichloro-8-fluoropyrido[4,3-d]pyrimidin-4-yl)piperidin-3-ol (208 mg, 582 μmol) in DCM (2 mL). The mixture was stirred for 2 hours. A separate solution of ((2R,7aS)-2-fluorotetrahydro-1H-pyrrolizin-7a(5H)-yl)methanol (111 mg, 699 μmol) in DCM (6 mL) was treated with LHMDS in THF (117 mg, 699 μL, 1.0 M, 699 μmol). The mixture was stirred at room temperature for 15 minutes and was then added dropwise in the reaction mixture containing 3-allyl-1-(2,7-dichloro-8-fluoropyrido[4,3-d]pyrimidin-4-yl)piperidin-3-ol. The reaction mixture was stirred at room temperature for 4 hours. The reaction mixture was concentrated and purified by reverse-phase flash column chromatography using a gradient of 10-100% B (A: water+0.1% formic acid; B: acetonitrile+0.1% formic acid) to afford 3-allyl-1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-ol (168 mg, 350 μmol, 60%).

MS (ESI): m/z (+)=480.3.

### Step D: Synthesis of 3-allyl-1-(7-(3-chloro-2-(hex-5-en-1-yl)-5-(methoxymethoxy)phenyl)-8-fluoro-2-(((2R,7aS)-2-fluorotetrahydro-1H-pyrrolizin-7a(5H)-yl)methoxy)pyrido[4,3-d]pyrimidin-4-yl)piperidin-3-ol

A solution of 3-allyl-1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-ol (150 mg, 313 μmol), diisopropyl (3-chloro-2-(hex-5-en-1-yl)-5-(methoxymethoxy)phenyl)boronate (171 mg, 447 μmol) cataCXium A Pd G3 (45.5 mg, 62.5 μmol), and potassium phosphate, tribasic (199 mg, 938 μmol) in 1,4-dioxane (7.0 mL) and water (1.8 mL) was degassed with nitrogen and warmed at 80° C. for 1 hour. The reaction mixture was concentrated onto celite and was purified by reverse-phase flash column chromatography using a gradient of 10-100% B (A: water+0.1% formic acid; B: acetonitrile+0.1% formic acid) to afford 3-allyl-1-(7-(3-chloro-2-(hex-5-en-1-yl)-5-(methoxymethoxy)phenyl)-8-fluoro-2-(((2R,7aS)-2-fluorotetrahydro-1H-pyrrolizin-7a(5H)-yl)methoxy)pyrido[4,3-d]pyrimidin-4-yl)piperidin-3-ol (107.9 mg, 154.5 mol, 49.4%).

MS (ESI): m/z (+)=698.5.

### Step E: Synthesis of 3³-chloro-2⁸-fluoro-2²-(((2R,7aS)-2-fluorotetrahydro-1H-pyrrolizin-7a(5H)-yl)methoxy)-3⁵-(methoxymethoxy)-2(4,7)-pyrido[4,3-d]pyrimidina-1(1,3)-piperidina-3(1,2)-benzenacyclodecaphan-8-en-1³-ol

A solution of 3-allyl-1-(7-(3-chloro-2-(hex-5-en-1-yl)-5-(methoxymethoxy)phenyl)-8-fluoro-2-(((2R,7aS)-2-fluorotetrahydro-1H-pyrrolizin-7a(5H)-yl)methoxy)pyrido[4,3-d]pyrimidin-4-yl)piperidin-3-ol (108 mg, 155 μmol) in DCM (30 mL) under nitrogen was treated with benzylidene(dichloro)(1,3-dimesityl-2-imidazolidinylidene)ruthenium-tricyclohexylphosphine (1:1) (19.7 mg, 23.2 μmol). The mixture was stirred at room temperature for 24 hours. Benzylidene(dichloro)(1,3-dimesityl-2-imidazolidinylidene)ruthenium-tricyclohexylphosphine (1:1) (19.7 mg, 23.2 μmol) was added, and the reaction mixture was stirred at 40° C. for 1 hour. The reaction mixture was concentrated to afford the crude product 3³-chloro-2⁸-fluoro-2²-(((2R,7aS)-2-fluorotetrahydro-1H-pyrrolizin-7a(5H)-yl)methoxy)-3⁵-(methoxymethoxy)-2(4,7)-pyrido[4,3-d]pyrimidina-1(1,3)-piperidina-3(1,2)-benzenacyclodecaphan-8-en-1³-ol (137 mg, 132%) which was carried forward into the next step without purification.

MS (ESI): m/z (+)=670.5.

### Step F: Synthesis of 3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclodecaphan-8-ene-1³,3⁵-diol

Crude 3³-chloro-2⁸-fluoro-2²-(((2R,7aS)-2-fluorotetrahydro-1H-pyrrolizin-7a(5H)-yl)methoxy)-3⁵-(methoxymethoxy)-2(4,7)-pyrido[4,3-d]pyrimidina-1(1,3)-piperidina-3(1,2)-benzenacyclodecaphan-8-en-1³-ol (137 mg, 206 μmol) was treated with HCl in 1,4-dioxane (225 mg, 1.54 mL, 4.0 M, 6.17 mmol). The mixture was stirred at room temperature for 1 hour. The reaction mixture was concentrated and was purified by HPLC (Method A) to afford 3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclodecaphan-8-ene-1³,3⁵-diol (7.8 mg, 12 μmol, 6.1%).

MS (ESI): m/z (+)=626.8.

### Step G: Synthesis of 3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclodecaphane-1³,3⁵-diol

A solution of 3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclodecaphan-8-ene-1³,3⁵-diol (1.2 mg, 1.9 μmol) in methanol (12 mL) was degassed with nitrogen. Pd/C (1.0 mg, 5 wt %, 0.48 μmol) was added and the reaction mixture was bubbled with hydrogen. The mixture was stirred under a hydrogen atmosphere for 18 hours.

The reaction mixture was filtered through celite and was concentrated to afford 3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclodecaphane-1³,3⁵-diol (1.2 mg, 1.9 μmol, 95%).

LCMS: Retention time=2.23 min, m/z (+)=626.8.

### Example 88. 3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclononaphane-1³,3⁵-diol (Compound 151a)

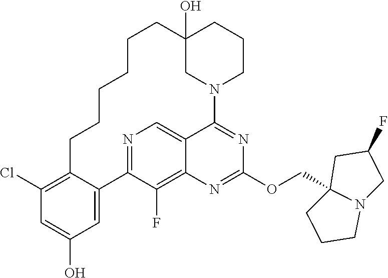 [SMILES: Oc1cc(Cl)c2c(c1)-c1ncc3c(nc(OC[C@@]45CCCN4C[C@H](F)C5)nc3c1F)N1CCCC(O)(CCCCCC2)C1]

3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclononaphane-1³,3⁵-diol was prepared in an analogous manner to Example 87.

LCMS: Retention time=2.12 min, m/z (+)=614.4.

### Example 89. 2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclodecaphan-3⁵-ol (Compound 143a)

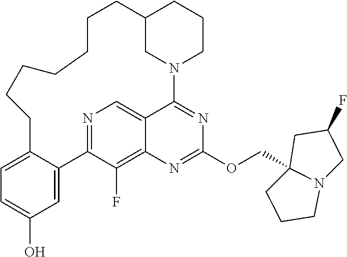 [SMILES: Oc1ccc2c(c1)-c1ncc3c(nc(OC[C@@]45CCCN4C[C@H](F)C5)nc3c1F)N1CCCC(CCCCCCC2)C1]

2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclodecaphan-35-ol was prepared in an analogous manner to Example 87.

LCMS: Retention time=2.36 min, m/z (+)=578.4.

### Example 90. (Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacycloundecaphan-1⁵-ol (Compound 153a)

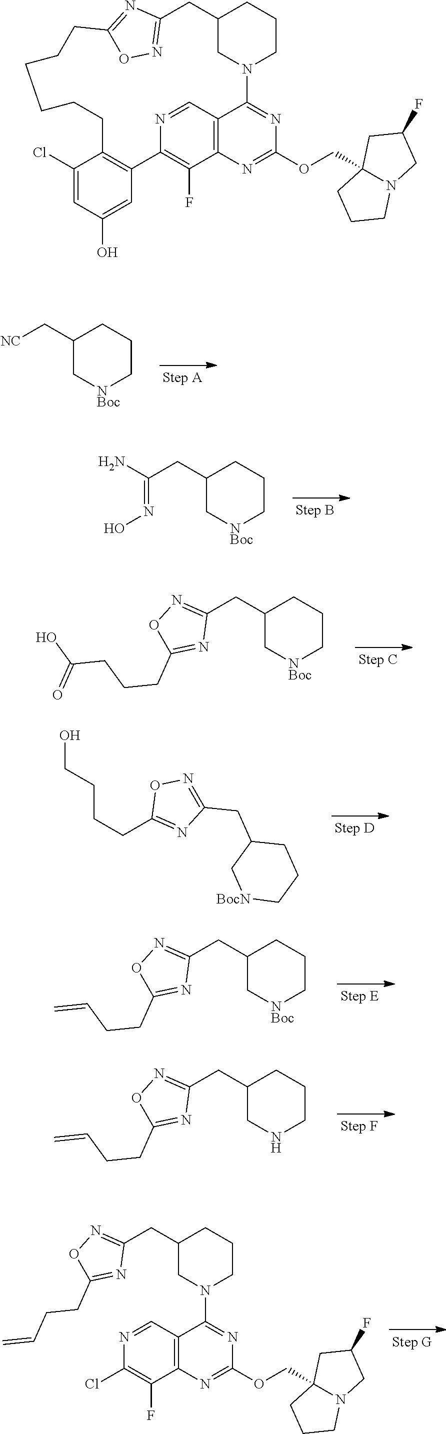

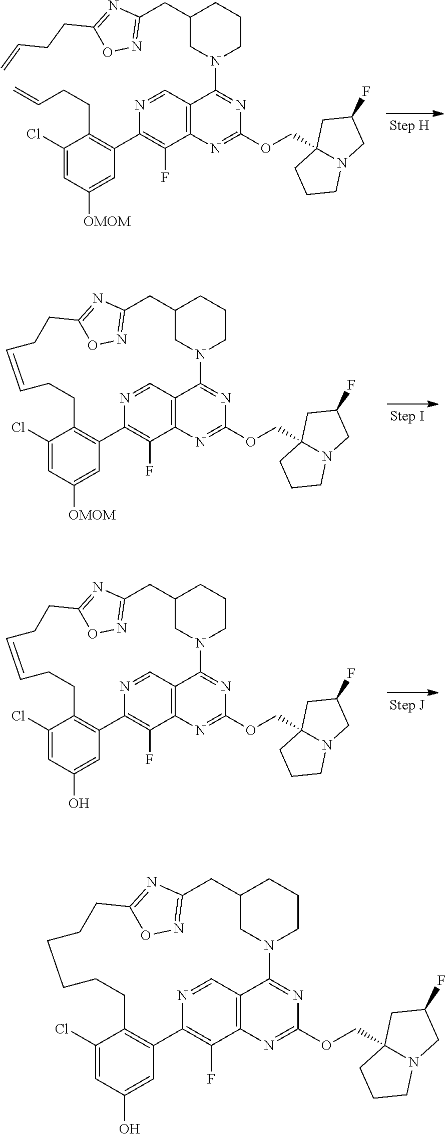

### Step A: Synthesis of tert-butyl 3-(2-amino-2-(hydroxyimino)ethyl)piperidine-1-carboxylate

A solution of tert-butyl 3-(cyanomethyl)piperidine-1-carboxylate (3.00 g, 13.4 mmol) in ethanol (5 mL) was treated with hydroxylamine hydrochloride (1.86 g, 26.7 mmol) and Cs₂CO₃ (8.72 g, 26.7 mmol) and was warmed at 70° C. for 5 days. The reaction mixture was diluted with ethyl acetate, washed with water and brine, dried (Na₂SO₄), filtered and concentrated to afford tert-butyl 3-(2-amino-2-(hydroxyimino)ethyl)piperidine-1-carboxylate (3.37 g, 13.1 mmol, 97.9%) MS (ESI): m/z (+)=258.3.

### Step B: Synthesis of 4-(3-((1-(tert-butoxycarbonyl)piperidin-3-yl)methyl)-1,2,4-oxadiazol-5-yl)butanoic acid

A solution of tert-butyl 3-(2-amino-2-(hydroxyimino)ethyl)piperidine-1-carboxylate (3.35 g, 13.0 mmol) and dihydro-2H-pyran-2,6(3H)-dione (1.49 g, 13.0 mmol) in Acetonitrile (40 mL) was treated with triethylamine (3.95 g, 5.44 mL, 39.1 mmol) and was stirred at 80° C. for 60 hours. The reaction mixture was concentrated and was purified by flash column chromatography (dichloromethane/methanol) to afford 4-(3-((1-(tert-butoxycarbonyl)piperidin-3-yl)methyl)-1,2,4-oxadiazol-5-yl)butanoic acid (3.03 g, 8.57 mmol, 65.9%).

MS (ESI): m/z (+)=354.3.

### Step C: Synthesis of tert-butyl 3-((5-(4-hydroxybutyl)-1,2,4-oxadiazol-3-yl)methyl)piperidine-1-carboxylate

A solution of 4-(3-((1-(tert-butoxycarbonyl)piperidin-3-yl)methyl)-1,2,4-oxadiazol-5-yl)butanoic acid (3030 mg, 8.57 mmol) in THF (10 mL) at room temperature was treated with borane tetrahydrofuran complex (811 mg, 9.43 mL, 1 molar, 9.43 mmol) and was stirred for 1 hour. Additional borane tetrahydrofuran complex (811 mg, 9.43 mL, 1 molar, 9.43 mmol) was added and the reaction mixture was stirred for 16 hours. The reaction mixture was diluted with ethyl acetate, washed with water and brine, dried (Na₂SO₄), filtered, and concentrated. The resulting residue was purified by reverse-phase flash column chromatography using a gradient of 10-100% B (A: water+0.1% formic acid; B: acetonitrile+0.1% formic acid) to afford tert-butyl 3-((5-(4-hydroxybutyl)-1,2,4-oxadiazol-3-yl)methyl)piperidine-1-carboxylate (1.94 g, 5.72 mmol, 66.7%).

MS (ESI): m/z (+)=340.2.

### Step D: Synthesis of tert-butyl 3-((5-(but-3-en-1-yl)-1,2,4-oxadiazol-3-yl)methyl)piperidine-1-carboxylate

A solution of tert-butyl 3-((5-(4-hydroxybutyl)-1,2,4-oxadiazol-3-yl)methyl)piperidine-1-carboxylate (970 mg, 2.86 mmol) and 1-nitro-2-selenocyanatobenzene (1.30 g, 5.72 mmol) in THF (15 mL) at room temperature was treated with Bu₃P (2.02 g, 2.47 mL, 10.0 mmol) and was stirred at room temperature for 3 hours. Hydrogen peroxide (3.24 g, 2.92 mL, 30% Wt, 28.6 mmol) was added and the reaction mixture was stirred at room temperature for 18 hours. The reaction mixture was diluted with ethyl acetate, washed with saturated aqueous NaHCO₃ and brine, dried (Na₂SO₄), filtered, and concentrated. The resulting residue was purified by flash column chromatography (ethyl acetate/heptane) to afford tert-butyl 3-((5-(but-3-en-1-yl)-1,2,4-oxadiazol-3-yl)methyl)piperidine-1-carboxylate (685 mg, 2.13 mmol, 74.6%).

MS (ESI): m/z (+)=322.2.

### Step E: Synthesis of 5-(but-3-en-1-yl)-3-(piperidin-3-ylmethyl)-1,2,4-oxadiazole

A solution of tert-butyl 3-((5-(but-3-en-1-yl)-1,2,4-oxadiazol-3-yl)methyl)piperidine-1-carboxylate (461 mg, 1.43 mmol) in dichloromethane (2 mL) at room temperature was treated with TFA (818 mg, 552 μL, 7.17 mmol) and was stirred at room temperature for 1 hour. The volatiles were removed under a stream of nitrogen and the resulting residue was dried under vacuum to afford 5-(but-3-en-1-yl)-3-(piperidin-3-ylmethyl)-1,2,4-oxadiazole, trifluoroacetic acid (481 mg, 1.43 mmol, 100%) which was used directly in the next step without purification.

MS (ESI): m/z (+)=222.2.

### Step F: Synthesis of 5-(but-3-en-1-yl)-3-((1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)methyl)-1,2,4-oxadiazole

A solution of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (276 mg, 628 μmol) and 5-(but-3-en-1-yl)-3-(piperidin-3-ylmethyl)-1,2,4-oxadiazole, trifluoroacetic acid (211 mg, 628 μmol) in DMSO (2 mL) was treated with DIEA (812 mg, 1.09 mL, 6.28 mmol) and was warmed at 50° C. for 16 hours. The mixture was concentrated and was purified by HPLC (Method A) to afford 5-(but-3-en-1-yl)-3-((1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)methyl)-1,2,4-oxadiazole (174 mg, 311 μmol, 49.5%) as an off-white solid.

MS (ESI): m/z (+)=560.5.

### Step G: Synthesis of 5-(but-3-en-1-yl)-3-((1-(7-(2-(but-3-en-1-yl)-3-chloro-5-(methoxymethoxy)phenyl)-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)methyl)-1,2,4-oxadiazole

A solution of 5-(but-3-en-1-yl)-3-((1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)methyl)-1,2,4-oxadiazole (130 mg, 232 μmol) and crude diisopropyl (2-(but-3-en-1-yl)-3-chloro-5-(methoxymethoxy)phenyl)boronate (123 mg, 348 μmol) in 1,4-Dioxane (3 mL) and Water (0.5 mL) was treated with Mesylate[(di(1-adamantyl)-n-butylphosphine)-2-(2′-amino-1,1′-biphenyl)]palladium(II) (16.9 mg, 23.2 μmol) and tripotassium phosphate (148 mg, 464 μL, 1.5 molar, 696 μmol) and the reaction mixture was warmed at 100° C. for 30 min in a microwave reactor. The reaction mixture was filtered and purified by HPLC (Method A) to afford 5-(but-3-en-1-yl)-3-((1-(7-(2-(but-3-en-1-yl)-3-chloro-5-(methoxymethoxy)phenyl)-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)methyl)-1,2,4-oxadiazole (52 mg, 69 μmol, 30%) as a white solid.

MS (ESI): m/z (+)=750.5.

### Step H: Synthesis of (5⁴Z,8Z)-1³-chloro-2⁸-fluoro-2²-(((2R,7aS)-2-fluorotetrahydro-1H-pyrrolizin-7a(5H)-yl)methoxy)-1⁵-(methoxymethoxy)-5(3,5)-oxadiazola-2(7,4)-pyrido[4,3-d]pyrimidina-3(1,3)-piperidina-1(1,2)-benzenacycloundecaphan-8-ene

A solution of 5-(but-3-en-1-yl)-3-((1-(7-(2-(but-3-en-1-yl)-3-chloro-5-(methoxymethoxy)phenyl)-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)methyl)-1,2,4-oxadiazole (52 mg, 69 μmol) in DCM (40 mL) was treated Dichloro(1,3-dimesityl-2-imidazolidinylidene)(2-isopropoxybenzylidene)ruthenium (17 mg, 28 μmol) and was degassed with nitrogen and was warmed at 40° C. for 16 hours. The reaction mixture was concentrated to afford crude (5⁴Z,8Z)-1³-chloro-2⁸-fluoro-2²-(((2R,7aS)-2-fluorotetrahydro-1H-pyrrolizin-7a(5H)-yl)methoxy)-15-(methoxymethoxy)-5(3,5)-oxadiazola-2(7,4)-pyrido[4,3-d]pyrimidina-3(1,3)-piperidina-1(1,2)-benzenacycloundecaphan-8-ene (50 mg, crude) which was used directly in the next step without purification.

MS (ESI): m/z (+)=722.6.

### Step I: Synthesis of (5⁴Z,8Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacycloundecaphan-8-en-1⁵-ol

A solution of crude (5⁴Z,8Z)-1³-chloro-2⁸-fluoro-2²-(((2R,7aS)-2-fluorotetrahydro-1H-pyrrolizin-7a(5H)-yl)methoxy)-1⁵-(methoxymethoxy)-5(3,5)-oxadiazola-2(7,4)-pyrido[4,3-d]pyrimidina-3(1,3)-piperidina-1(1,2)-benzenacycloundecaphan-8-ene (50 mg, 69 μmol) was dissolved in 2 mL 20% TFA in dichloromethane and was stirred at room temperature for 2 hours. The reaction mixture was concentrated and was purified by HPLC (Method A) to afford (5⁴Z,8Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacycloundecaphan-8-en-1⁵-ol (8.5 mg, 18%).

MS (ESI): m/z (+)=678.8.

### Step J: Synthesis of (Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacycloundecaphan-1⁵-ol

A solution of (5⁴Z,8Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacycloundecaphan-8-en-1⁵-ol (5.6 mg, 8.3 μmol) and Pd/C (1.8 mg, 10% Wt, 1.7 μmol) in ethyl acetate (2 mL) was bubbled with hydrogen for 30 minutes and was stirred under a hydrogen atmosphere for 20 hours. The reaction mixture was filtered and was purified by HPLC (Method A) to afford (Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacycloundecaphan-1⁵-ol (0.4 mg, 0.6 μmol, 7%).

LCMS: Retention time=2.27 min, m/z (+)=680.5.

### ADDITIONAL EXAMPLES

### Example 1. Synthesis of 3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclodecaphan-3⁵-ol (Compound 108a)

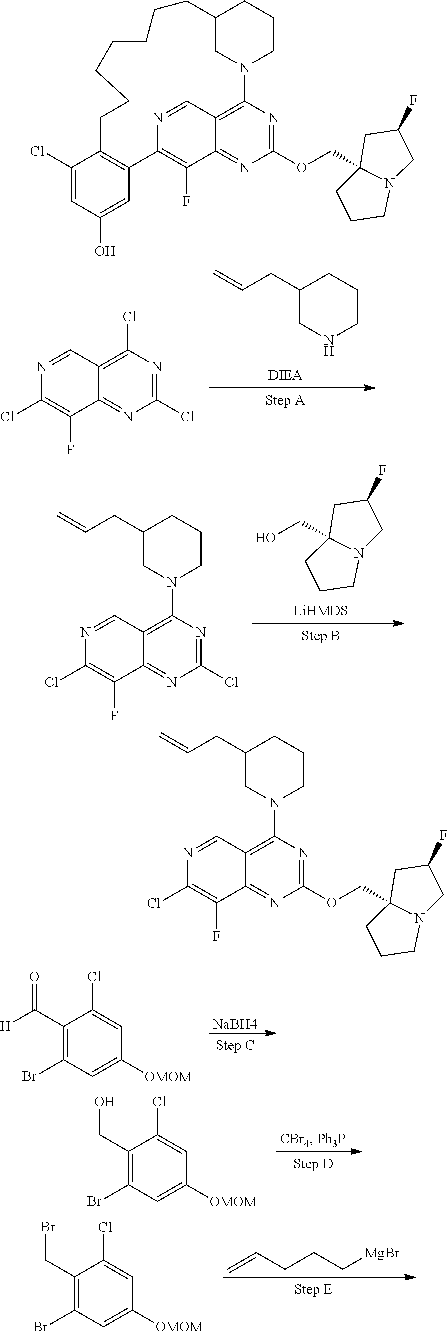

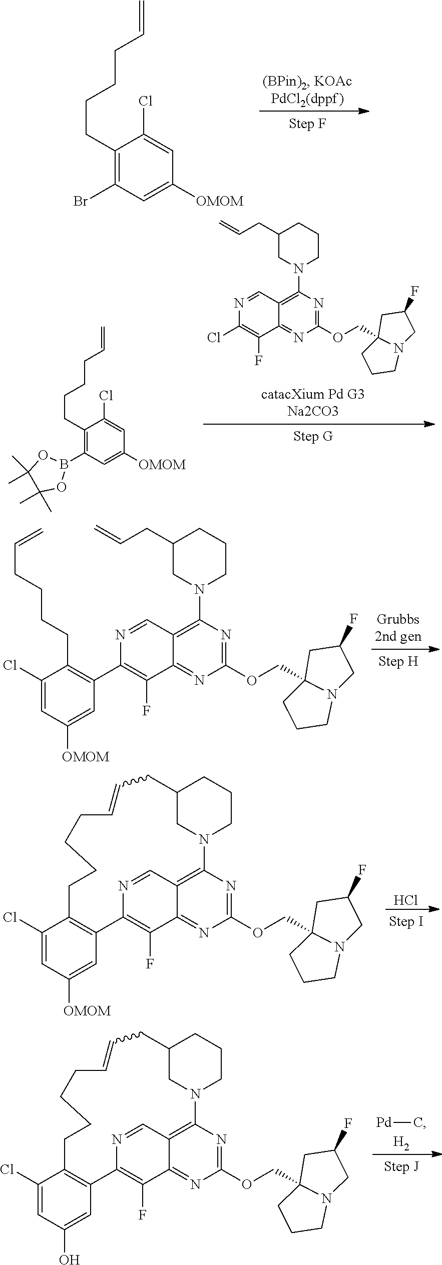

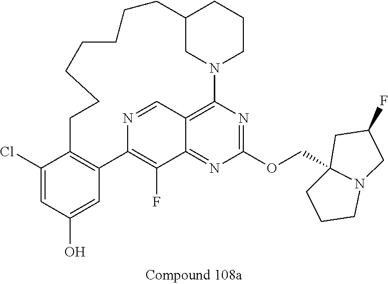 [SMILES: Oc1cc(Cl)c2c(c1)-c1ncc3c(nc(OC[C@@]45CCCN4C[C@H](F)C5)nc3c1F)N1CCCC(CCCCCCC2)C1]

### Step A: Synthesis of 4-(3-allylpiperidin-1-yl)-2,7-dichloro-8-fluoropyrido[4,3-d]pyrimidine

A solution of 2,4,7-trichloro-8-fluoropyrido[4,3-d]pyrimidine (400 mg, 1 equiv., 1.58 mmol) in DCM (6.00 mL) under nitrogen was cooled to −78° C. 3-Allylpiperidine (198 mg, 1 equiv., 1.58 mmol) was added and the reaction was stirred for 5 minutes. DIPEA (410 mg, 552 μL, 2.0 equiv., 3.17 mmol) was added dropwise. The reaction mixture was stirred at −78° C. for 1 hour and was allowed to slowly warm to room temperature overnight. The reaction mixture was concentrated and was purified by flash column chromatography (ethyl acetate/heptane) to afford 4-(3-allylpiperidin-1-yl)-2,7-dichloro-8-fluoropyrido[4,3-d]pyrimidine (496 mg, 91.7%).

MS (ESI): m/z (+)=341.1

### Step B: Synthesis of 4-(3-allylpiperidin-1-yl)-7-chloro-8-fluoro-2-(((2R,7aS)-2-fluorotetrahydro-1H-pyrrolizin-7a(5H)-yl)methoxy)pyrido[4,3-d]pyrimidine

A solution of ((2R,7aS)-2-fluorotetrahydro-1H-pyrrolizin-7a(5H)-yl)methanol (255 mg, 1.1 equiv., 1.60 mmol) in 1,4-dioxane (10.0 mL) was treated with LiHMDS (365 mg, 2.18 mL, 1.5 equiv., 2.18 mmol). The mixture was stirred for 5 minutes. 4-(3-Allylpiperidin-1-yl)-2,7-dichloro-8-fluoropyrido[4,3-d]pyrimidine (496 mg, 1.0 equiv., 1.45 mmol) was added and the reaction mixture was warmed at 70° C. for 18 hours. The reaction mixture was concentrated and was purified by reverse-phase flash column chromatography using a gradient of 10-100% B (A: water+0.1% formic acid; B: acetonitrile+0.1% formic acid) to afford 4-(3-allylpiperidin-1-yl)-7-chloro-8-fluoro-2-(((2R,7aS)-2-fluorotetrahydro-1H-pyrrolizin-7a(5H)-yl)methoxy)pyrido[4,3-d]pyrimidine (550 mg, 82%).

MS (ESI): m/z (+)=464.5

### Step C: Synthesis of (2-bromo-6-chloro-4-(methoxymethoxy)phenyl)methanol

A solution of 2-bromo-6-chloro-4-(methoxymethoxy)benzaldehyde (2.00 g, 1 equiv., 7.16 mmol) in MeOH (20 mL) at room temperature was treated portionwise with NaBH₄ (271 mg, 1 equiv., 7.16 mmol). The mixture was stirred at room temperature for 1 hour. The reaction mixture was diluted with water and EtOAc, washed with brine, dried (Na₂SO₄), filtered, and concentrated. The resulting residue was purified by flash column chromatography (ethyl acetate/heptane) to afford (2-bromo-6-chloro-4-(methoxymethoxy)phenyl)methanol (1.88 g, 6.68 mmol, 93.3%).

¹H NMR (400 MHz, DMSO-d6) δ 7.28 (d, J=2.4 Hz, 1H), 7.17 (d, J=2.5 Hz, 1H), 5.24 (s, 2H), 5.07 (t, J=5.2 Hz, 1H), 4.64 (d, J=5.2 Hz, 2H), 3.37 (s, 3H).

### Step D: Synthesis of 1-bromo-2-(bromomethyl)-3-chloro-5-(methoxymethoxy)benzene

A solution of (2-bromo-6-chloro-4-(methoxymethoxy)phenyl)methanol (1.85 g, 1 equiv., 6.57 mmol) and CBr₄ (2.40 g, 1.1 equiv., 7.23 mmol) in DCM (20 mL) at room temperature was treated with Ph₃P (1.90 g, 1.1 equiv., 7.23 mmol). The mixture was stirred for 1 hour. The reaction mixture was diluted with DCM and was washed with water and brine, dried (Na₂SO₄), filtered, and concentrated. The resulting residue was purified by flash column chromatography (ethyl acetate/heptane) to afford 1-bromo-2-(bromomethyl)-3-chloro-5-(methoxymethoxy)benzene (1.70 g, 4.94 mmol, 75.1%).

¹H NMR (400 MHz, DMSO-d6) δ 7.37 (dd, J=3.4, 2.5 Hz, 1H), 7.26 (dd, J=3.7, 2.5 Hz, 1H), 5.27 (d, J=1.1 Hz, 2H), 4.78 (s, 2H), 3.38 (s, 3H).

### Step E: Synthesis of 1-bromo-3-chloro-2-(hex-5-en-1-yl)-5-(methoxymethoxy)benzene

A solution of 5-bromopent-1-ene (528 mg, 1.16 equiv., 3.54 mmol) in THF (3.0 mL) under nitrogen at −78° C. was treated with magnesium (88.8 mg, 1.2 equiv., 3.66 mmol). The mixture was stirred for 10 minutes. The reaction mixture was allowed to warm to room temperature and a small crystal of iodine was added. The reaction mixture was stirred vigorously for 2 hours. A solution of 1-bromo-2-(bromomethyl)-3-chloro-5-(methoxymethoxy)benzene (1.0492 g, 1 equiv., 3.0462 mmol) in THF (3.0 mL) was added dropwise. The reaction mixture was stirred at room temperature for 16 hours. The reaction mixture was quenched by the slow addition of 10% water in THF (1.10 g, 1.10 mL, 10 wt %, 2 equiv., 6.09 mmol) while stirring. The reaction mixture was filtered and the filtrate was concentrated and purified by flash column chromatography (ethyl acetate/heptane) to afford 1-bromo-3-chloro-2-(hex-5-en-1-yl)-5-(methoxymethoxy)benzene (130 mg, 13%).

¹H NMR (400 MHz, CDCl₃) δ 7.18 (d, J=2.6 Hz, 1H), 7.04 (d, J=2.6 Hz, 1H), 5.91-5.76 (m, 1H), 5.11 (s, 2H), 5.05-4.99 (m, 1H), 4.97-4.92 (m, 1H), 3.46 (s, 3H), 2.90-2.82 (m, 2H), 2.16-2.08 (m, 2H), 1.58-1.47 (m, 4H).

### Step F: Synthesis of 2-(3-chloro-2-(hex-5-en-1-yl)-5-(methoxymethoxy)phenyl)-4,4,5,5-tetramethyl-1,3,2-dioxaborolane

A solution of 1-bromo-3-chloro-2-(hex-5-en-1-yl)-5-(methoxymethoxy)benzene (23.0 mg, 1 equiv., 68.9 μmol), PdCl₂(dppf)-CH₂Cl₂ adduct (11.3 mg, 0.2 equiv., 13.8 μmol), potassium acetate (10.1 mg, 1.5 equiv., 103 μmol) and 4,4,4′,4′,5,5,5′,5′-octamethyl-2,2′-bi(1,3,2-dioxaborolane) (17.5 mg, 1 equiv., 68.9 μmol) in 1,4-dioxane (1000 μL) was degassed with nitrogen and was warmed at 100° C. for 1 hour. The reaction mixture was cooled to room temperature and the resulting reaction mixture was concentrated to afford crude 2-(3-chloro-2-(hex-5-en-1-yl)-5-(methoxymethoxy)phenyl)-4,4,5,5-tetramethyl-1,3,2-dioxaborolane which was used directly in the next step without purification.

### Step G: Synthesis of 4-(3-allylpiperidin-1-yl)-7-(3-chloro-2-(hex-5-en-1-yl)-5-(methoxymethoxy)phenyl)-8-fluoro-2-(((2R,7aS)-2-fluorotetrahydro-1H-pyrrolizin-7a(5H)-yl)methoxy)pyrido[4,3-d]pyrimidine

Crude 2-(3-chloro-2-(hex-5-en-1-yl)-5-(methoxymethoxy)phenyl)-4,4,5,5-tetramethyl-1,3,2-dioxaborolane from the previous step, 4-(3-allylpiperidin-1-yl)-7-chloro-8-fluoro-2-(((2R,7aS)-2-fluorotetrahydro-1H-pyrrolizin-7a(5H)-yl)methoxy)pyrido[4,3-d]pyrimidine (54 mg, 1 equiv., 0.12 mmol), cataCXium (R) A Pd G3 (17 mg, 0.2 equiv., 23 μmol), and Na2CO3 (37 mg, 3 equiv., 0.35 mmol) in 1,4-dioxane (2.0 mL) and Water (0.5 mL) was degassed with nitrogen and was warmed at 110° C. for 16 hours. The reaction mixture was concentrated and was purified by HPLC (Method A) to afford 4-(3-allylpiperidin-1-yl)-7-(3-chloro-2-(hex-5-en-1-yl)-5-(methoxymethoxy)phenyl)-8-fluoro-2-(((2R,7aS)-2-fluorotetrahydro-1H-pyrrolizin-7a(5H)-yl)methoxy)pyrido[4,3-d]pyrimidine (3.7 mg, 4.7% over 2 steps).

MS (ESI): m/z (+)=682.5

### Step H: Synthesis of 3³-chloro-2⁸-fluoro-2²-(((2R,7aS)-2-fluorotetrahydro-1H-pyrrolizin-7a(5H)-yl)methoxy)-3⁵-(methoxymethoxy)-2(4,7)-pyrido[4,3-d]pyrimidina-1(1,3)-piperidina-3(1,2)-benzenacyclodecaphan-8-ene

A solution of 4-(3-allylpiperidin-1-yl)-7-(3-chloro-2-(hex-5-en-1-yl)-5-(methoxymethoxy)phenyl)-8-fluoro-2-(((2R,7aS)-2-fluorotetrahydro-1H-pyrrolizin-7a(5H)-yl)methoxy)pyrido[4,3-d]pyrimidine (0.8 mg, 1 equiv., 1 μmol) and Grubbs Catalyst 2nd Gen (0.4 mg, 0.4 equiv., 0.5 μmol) in Toluene (1.5 mL) was degassed with nitrogen and was warmed at 100° C. for 24 hour. The reaction mixture was concentrated and purified by HPLC (Method A) to afford 3³-chloro-2⁸-fluoro-2²-(((2R,7aS)-2-fluorotetrahydro-1H-pyrrolizin-7a(5H)-yl)methoxy)-3⁵-(methoxymethoxy)-2(4,7)-pyrido[4,3-d]pyrimidina-1(1,3)-piperidina-3(1,2)-benzenacyclodecaphan-8-ene as a mixture of E and Z isomers.

MS (ESI): m/z (+)=654.4

### Step I: Synthesis of 3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclodecaphan-8-en-3⁵-ol

3³-chloro-2⁸-fluoro-2²-(((2R,7aS)-2-fluorotetrahydro-1H-pyrrolizin-7a(5H)-yl)methoxy)-3⁵-(methoxymethoxy)-2(4,7)-pyrido[4,3-d]pyrimidina-1(1,3)-piperidina-3(1,2)-benzenacyclodecaphan-8-ene is reacted under standard conditions for MOM deprotection to afford 3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclodecaphan-8-en-3⁵-ol.

### Step J: Synthesis of 3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclodecaphan-3⁵-ol

3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclodecaphan-8-en-3⁵-ol is reduced under standard conditions to afford 3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclodecaphan-35-ol.

### Example 2: Synthesis of (3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclononaphan-3⁵-ol) (Compound 107a)

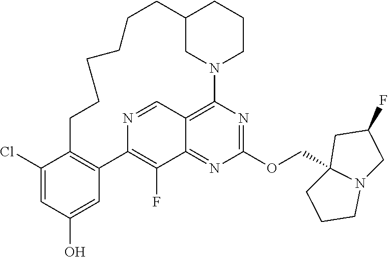 [SMILES: Oc1cc(Cl)c2c(c1)-c1ncc3c(nc(OC[C@@]45CCCN4C[C@H](F)C5)nc3c1F)N1CCCC(CCCCCC2)C1]

(3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacyclononaphan-3⁵-ol) is prepared in an analogous manner to Example 1 by using 3-vinylpiperidine in Step A.

### Example 3: Synthesis of (3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacycloundecaphan-3⁵-ol) (Compound 109a)

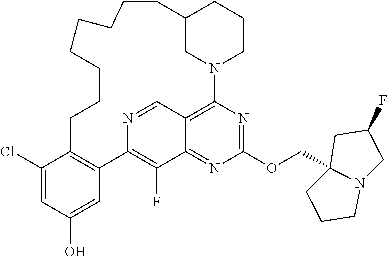 [SMILES: Oc1cc(Cl)c2c(c1)-c1ncc3c(nc(OC[C@@]45CCCN4C[C@H](F)C5)nc3c1F)N1CCCC(CCCCCCCC2)C1]

(3³-chloro-2⁸-fluoro-2²-(((2R,7aS)-2-fluorotetrahydro-1H-pyrrolizin-7a(5H)-yl)methoxy)-2(4,7)-pyrido[4,3-d]pyrimidina-1(1,3)-piperidina-3(1,2)-benzenacycloundecaphan-3⁵-ol) is prepared in an analogous manner to Example 1 by using 3-(but-3-en-1-yl)piperidine in Step A.

### Example 4. Synthesis of (Z)-1⁷,2⁸-difluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,8)-naphthalenacycloheptaphan-1³-ol (Compound 101a)

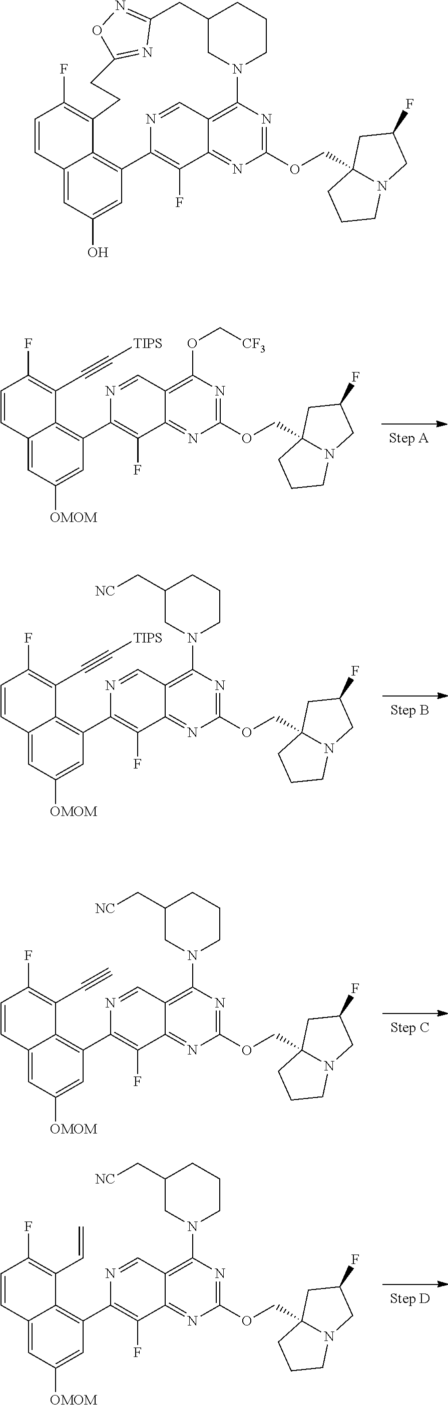

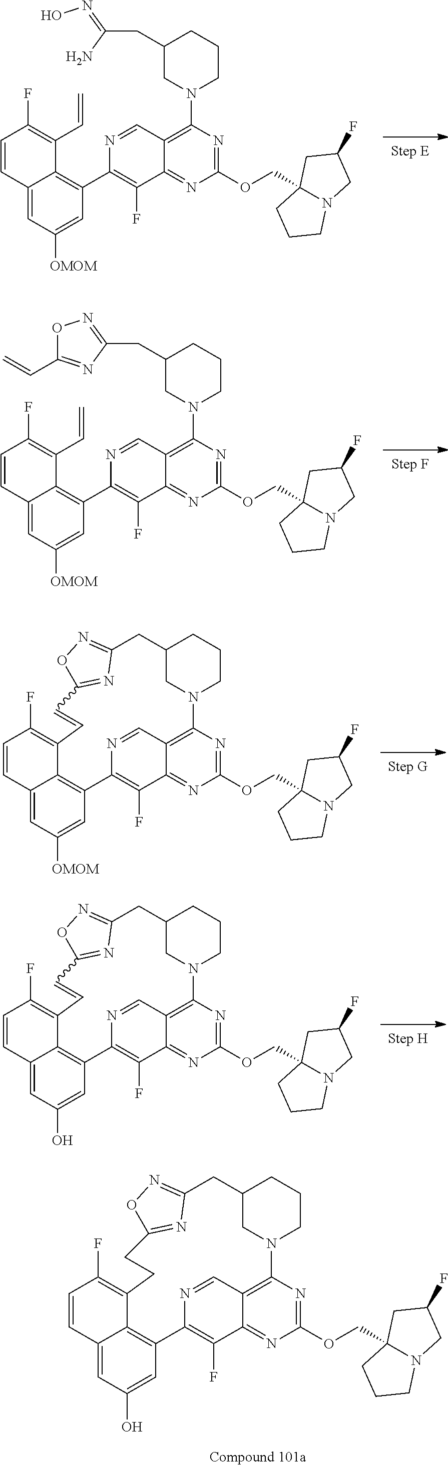

### Step A: Synthesis of 2-(1-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)acetonitrile

8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine is subjected to SNAr with 2-(piperidin-3-yl)acetonitrile to obtain 2-(1-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)acetonitrile.

### Step B: Synthesis of 2-(1-(7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)acetonitrile

2-(1-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)acetonitrile is silyl deprotected under standard conditions to obtain 2-(1-(7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)acetonitrile.

### Step C: Synthesis of 2-(1-(8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)acetonitrile

2-(1-(7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)acetonitrile is reduced under standard conditions to obtain 2-(1-(8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)acetonitrile.

### Step D: Synthesis of (Z)-2-(1-(8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)-N′-hydroxyacetimidamide

2-(1-(8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)acetonitrile is reacted with hydroxylamine to obtain (Z)-2-(1-(8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)-N′-hydroxyacetimidamide.

### Step E: Synthesis of 3-((1-(8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)methyl)-5-vinyl-1,2,4-oxadiazole

(Z)-2-(1-(8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)-N′-hydroxyacetimidamide is treated with acrylic acid under standard conditions to obtain 3-((1-(8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)methyl)-5-vinyl-1,2,4-oxadiazole.

### Step F: Synthesis of (5⁴Z)-1⁷,2⁸-difluoro-2²-(((2R,7aS)-2-fluorotetrahydro-1H-pyrrolizin-7a(5H)-yl)methoxy)-1³-(methoxymethoxy)-5(3,5)-oxadiazola-2(7,4)-pyrido[4,3-d]pyrimidina-3(1,3)-piperidina-1(1,8)-naphthalenacycloheptaphan-6-ene

3-((1-(8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)methyl)-5-vinyl-1,2,4-oxadiazole is reacted under ring closing metathesis conditions to afford (5⁴Z)-1⁷,2⁸-difluoro-2²-(((2R,7aS)-2-fluorotetrahydro-1H-pyrrolizin-7a(5H)-yl)methoxy)-1³-(methoxymethoxy)-5(3,5)-oxadiazola-2(7,4)-pyrido[4,3-d]pyrimidina-3(1,3)-piperidina-1(1,8)-naphthalenacycloheptaphan-6-ene.

### Step G: Synthesis of (5⁴Z)-1⁷,2⁸-difluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,8)-naphthalenacycloheptaphan-6-en-1³-ol

(5⁴Z)-1⁷,2⁸-difluoro-2²-(((2R,7aS)-2-fluorotetrahydro-1H-pyrrolizin-7a(5H)-yl)methoxy)-1³-(methoxymethoxy)-5(3,5)-oxadiazola-2(7,4)-pyrido[4,3-d]pyrimidina-3(1,3)-piperidina-1(1,8)-naphthalenacycloheptaphan-6-ene is deprotected under standard conditions to afford (5⁴Z)-1⁷,2⁸-difluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,8)-naphthalenacycloheptaphan-6-en-1³-ol.

### Step H: Synthesis of (Z)-1⁷,2⁸-difluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,8)-naphthalenacycloheptaphan-1³-ol

(5⁴Z)-1⁷,2⁸-difluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,8)-naphthalenacycloheptaphan-6-en-1³-ol is reduced under standard conditions to obtain (Z)-1⁷,2⁸-difluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,8)-naphthalenacycloheptaphan-1³-ol.

### Example 5. Synthesis of (Z)-1⁷,2⁸-difluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,8)-naphthalenacyclooctaphan-1³-ol (Compound 102a)

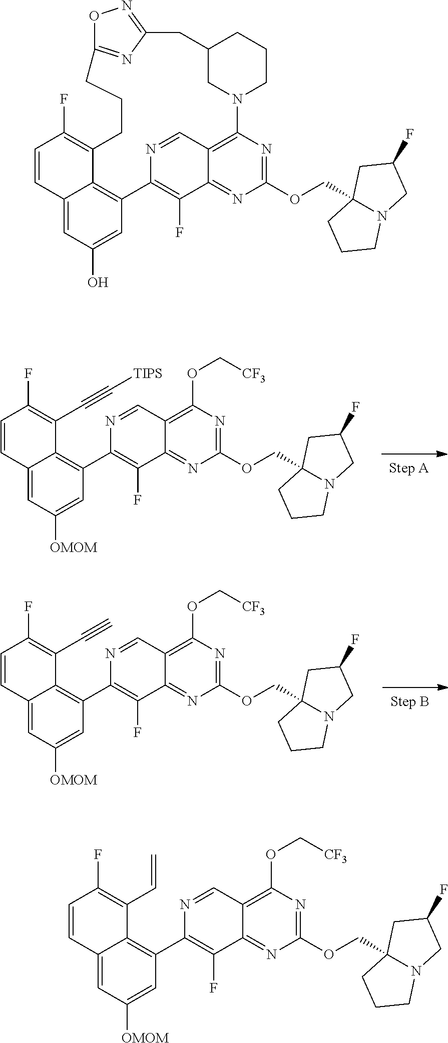

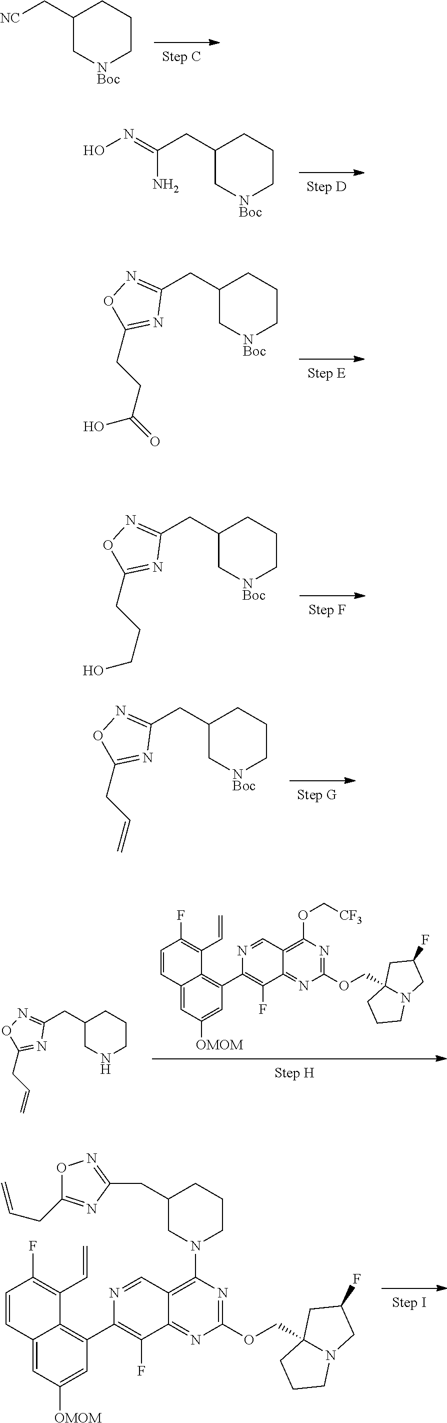

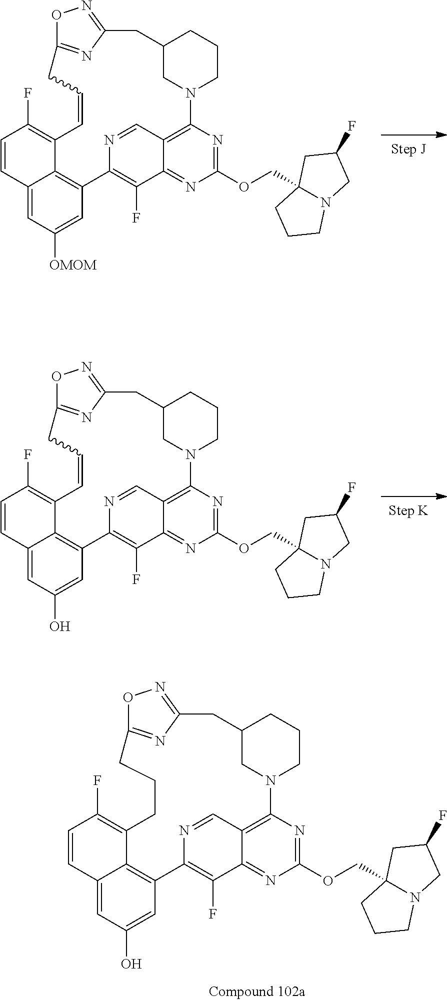

### Step A: Synthesis of 7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine

8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine is silyl deprotected under standard conditions to obtain 7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine.

### Step B: Synthesis of 8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine

7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine is reduced under standard conditions to obtain 8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine.

### Step C: Synthesis of tert-butyl (Z)-3-(2-amino-2-(hydroxyimino)ethyl)piperidine-1-carboxylate

tert-butyl 3-(cyanomethyl)piperidine-1-carboxylate is reacted with hydroxylamine under standard conditions to obtain tert-butyl (Z)-3-(2-amino-2-(hydroxyimino)ethyl)piperidine-1-carboxylate.

### Step D: Synthesis of 3-(3-((1-(tert-butoxycarbonyl)piperidin-3-yl)methyl)-1,2,4-oxadiazol-5-yl)propanoic acid

tert-butyl (Z)-3-(2-amino-2-(hydroxyimino)ethyl)piperidine-1-carboxylate is cyclized with dihydrofuran-2,5-dione to obtain 3-(3-((1-(tert-butoxycarbonyl)piperidin-3-yl)methyl)-1,2,4-oxadiazol-5-yl)propanoic acid.

### Step E: Synthesis of tert-butyl 3-((5-(3-hydroxypropyl)-1,2,4-oxadiazol-3-yl)methyl)piperidine-1-carboxylate

3-(3-((1-(tert-butoxycarbonyl)piperidin-3-yl)methyl)-1,2,4-oxadiazol-5-yl)propanoic acid is reduced under standard conditions to obtain tert-butyl 3-((5-(3-hydroxypropyl)-1,2,4-oxadiazol-3-yl)methyl)piperidine-1-carboxylate.

### Step F: Synthesis of tert-butyl 3-((5-allyl-1,2,4-oxadiazol-3-yl)methyl)piperidine-1-carboxylate

tert-butyl 3-((5-(3-hydroxypropyl)-1,2,4-oxadiazol-3-yl)methyl)piperidine-1-carboxylate is dehydrated under standard conditions to obtain tert-butyl 3-((5-allyl-1,2,4-oxadiazol-3-yl)methyl)piperidine-1-carboxylate.

### Step G: Synthesis of 5-allyl-3-(piperidin-3-ylmethyl)-1,2,4-oxadiazole

tert-butyl 3-((5-allyl-1,2,4-oxadiazol-3-yl)methyl)piperidine-1-carboxylate is deprotected under standard conditions to obtain 5-allyl-3-(piperidin-3-ylmethyl)-1,2,4-oxadiazole.

### Step H: Synthesis of 5-allyl-3-((1-(8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)methyl)-1,2,4-oxadiazole

5-allyl-3-(piperidin-3-ylmethyl)-1,2,4-oxadiazole is subjected to SNAr conditions with 8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine to obtain 5-allyl-3-((1-(8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)methyl)-1,2,4-oxadiazole.

### Step I: Synthesis of (5⁴Z)-1⁷,2⁸-difluoro-2²-(((2R,7aS)-2-fluorotetrahydro-1H-pyrrolizin-7a(5H)-yl)methoxy)-1³-(methoxymethoxy)-5(3,5)-oxadiazola-2(7,4)-pyrido[4,3-d]pyrimidina-3(1,3)-piperidina-1(1,8)-naphthalenacyclooctaphan-7-ene

5-allyl-3-((1-(8-fluoro-7-(7-fluoro-3-(methoxymethoxy)-8-vinylnaphthalen-1-yl)-2-(((2R,7aS)-2-fluorotetrahydro-1H-pyrrolizin-7a(5H)-yl)methoxy)pyrido[4,3-d]pyrimidin-4-yl)piperidin-3-yl)methyl)-1,2,4-oxadiazole is reacted under ring closing metathesis conditions to afford (5⁴Z)-1⁷,2⁸-difluoro-2²-(((2R,7aS)-2-fluorotetrahydro-1H-pyrrolizin-7a(5H)-yl)methoxy)-1³-(methoxymethoxy)-5(3,5)-oxadiazola-2(7,4)-pyrido[4,3-d]pyrimidina-3(1,3)-piperidina-1(1,8)-naphthalenacyclooctaphan-7-ene.

### Step J: Synthesis of (5⁴Z)-1⁷,2⁸-difluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,8)-naphthalenacyclooctaphan-7-en-13-ol

(5⁴Z)-1⁷,2⁸-difluoro-2²-(((2R,7aS)-2-fluorotetrahydro-1H-pyrrolizin-7a(5H)-yl)methoxy)-1³-(methoxymethoxy)-5(3,5)-oxadiazola-2(7,4)-pyrido[4,3-d]pyrimidina-3(1,3)-piperidina-1(1,8)-naphthalenacyclooctaphan-7-ene is deprotected under standard conditions to obtain (5⁴Z)-1⁷,2⁸-difluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,8)-naphthalenacyclooctaphan-7-en-1³-ol.

### Step K: Synthesis of (Z)-1⁷,2⁸-difluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,8)-naphthalenacyclooctaphan-1³-ol

(5⁴Z)-1⁷,2⁸-difluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,8)-naphthalenacyclooctaphan-7-en-1³-ol is reduced under standard conditions to afford (Z)-1⁷,2⁸-difluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,8)-naphthalenacyclooctaphan-1³-ol.

### Example 6. (Z)-1⁷,2⁸-difluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,8)-naphthalenacyclononaphan-1³-ol (Compound 103a)

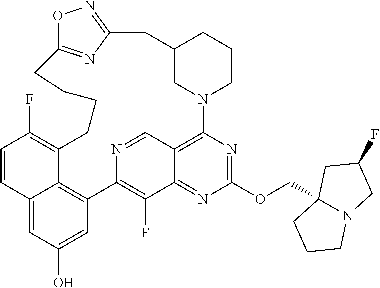 [SMILES: Oc1cc2c3c(c(F)ccc3c1)CCCCc1nc(no1)CC1CCCN(C1)c1nc(OC[C@@]34CCCN3C[C@H](F)C4)nc3c(F)c-2ncc13]

(Z)-1⁷,2⁸-difluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,8)-naphthalenacyclononaphan-1³-ol is prepared in an analogous manner to Example 5 by using dihydro-2H-pyran-2,6(3H)-dione in Step D.

### Example 7. Synthesis of (Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacycloheptaphan-1⁵-ol (Compound 104a)

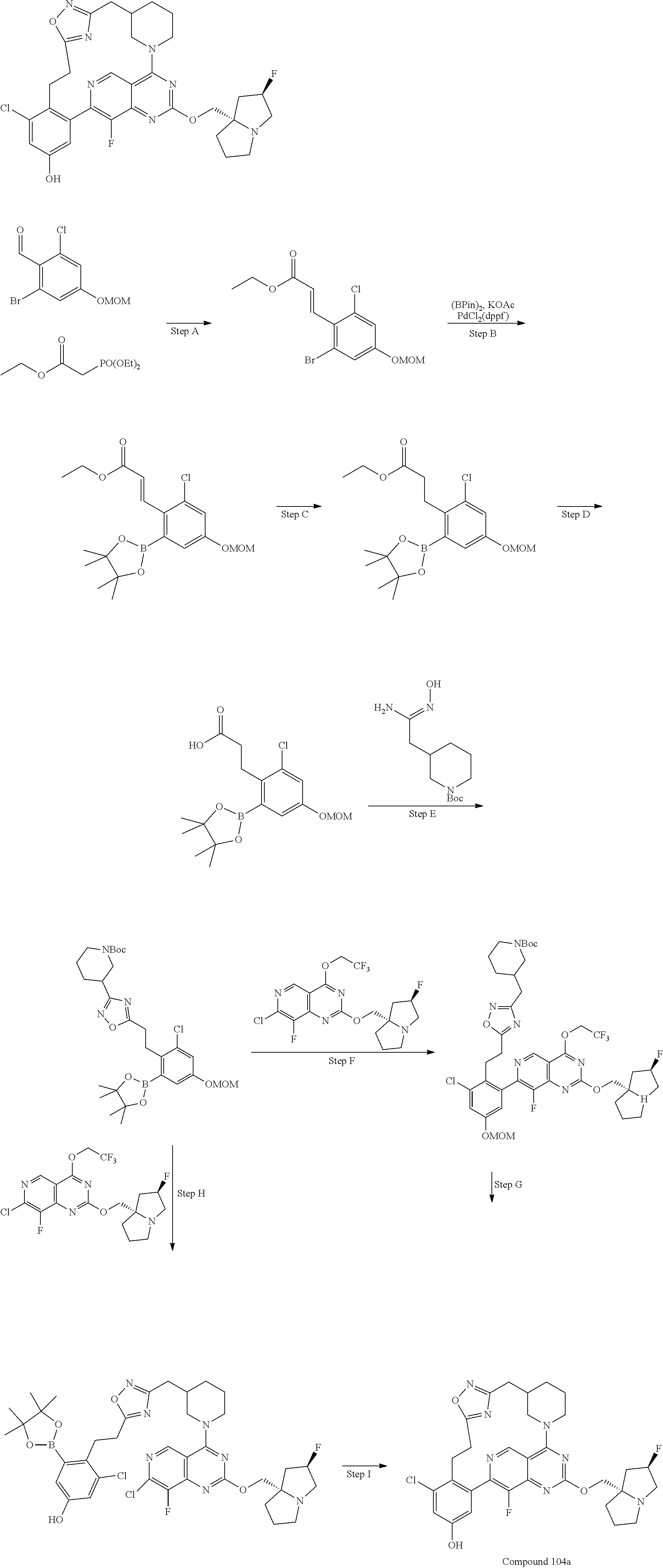

### Step A: Synthesis of ethyl (E)-3-(2-bromo-6-chloro-4-(methoxymethoxy)phenyl)acrylate

A solution of 2-bromo-6-chloro-4-(methoxymethoxy)benzaldehyde (2.00 g, 1 equiv., 7.16 mmol) and ethyl 2-(diethoxyphosphoryl)acetate (1.76 g, 1.1 equiv., 7.87 mmol) in THF (20 mL) was treated portionwise with NaH (372 mg, 60 wt %, 1.3 equiv., 9.30 mmol). The mixture was stirred for 1 hour. The reaction mixture was diluted with EtOAc, washed with water and brine, dried (Na₂SO₄), filtered, and concentrated to afford ethyl (E)-3-(2-bromo-6-chloro-4-(methoxymethoxy)phenyl)acrylate (2.45 g, 98%).

¹H NMR (400 MHz, DMSO-d6) δ 7.60 (d, J=16.3 Hz, 1H), 7.42 (d, J=2.5 Hz, 1H), 7.29 (d, J=2.5 Hz, 1H), 6.48 (d, J=16.3 Hz, 1H), 5.29 (s, 2H), 4.22 (q, J=7.1 Hz, 2H), 3.38 (s, 3H), 1.27 (t, J=7.1 Hz, 3H)

### Step B: Synthesis of ethyl (E)-3-(2-chloro-4-(methoxymethoxy)-6-(4,4,5,5-tetramethyl-1,3,2-dioxaborolan-2-yl)phenyl)acrylate

A solution of ethyl (E)-3-(2-bromo-6-chloro-4-(methoxymethoxy)phenyl)acrylate (13.8 g, 1 equiv., 39.3 mmol), Bis(pinacolato)diborane (20.0 g, 2.0 equiv., 78.7 mmol), potassium acetate (11.6 g, 3.0 equiv., 118 mmol), and Pd(dppf)Cl₂·DCM (3.21 g, 0.1 equiv., 3.93 mmol) in 1,4-dioxane (100 mL) was warmed at 100° C. for 2 hours. The reaction mixture was diluted with ethyl acetate, washed with water and brine, dried (MgSO₄), filtered, and concentrated. The resulting residue was purified by flash column chromatography (ethyl acetate/heptane) to afford ethyl (E)-3-(2-chloro-4-(methoxymethoxy)-6-(4,4,5,5-tetramethyl-1,3,2-dioxaborolan-2-yl)phenyl)acrylate (15.0 g, 96%).

MS (ESI): m/z (+)=397.2

### Step C: Synthesis of ethyl 3-(2-chloro-4-(methoxymethoxy)-6-(4,4,5,5-tetramethyl-1,3,2-dioxaborolan-2-yl)phenyl)propanoate

Ethyl (E)-3-(2-chloro-4-(methoxymethoxy)-6-(4,4,5,5-tetramethyl-1,3,2-dioxaborolan-2-yl)phenyl)acrylate is reduced using standard conditions to obtain ethyl 3-(2-chloro-4-(methoxymethoxy)-6-(4,4,5,5-tetramethyl-1,3,2-dioxaborolan-2-yl)phenyl)propanoate.

### Step D: Synthesis of 3-(2-chloro-4-(methoxymethoxy)-6-(4,4,5,5-tetramethyl-1,3,2-dioxaborolan-2-yl)phenyl)propanoic acid

Ethyl 3-(2-chloro-4-(methoxymethoxy)-6-(4,4,5,5-tetramethyl-1,3,2-dioxaborolan-2-yl)phenyl)propanoate is hydrolyzed using standard conditions to obtain 3-(2-chloro-4-(methoxymethoxy)-6-(4,4,5,5-tetramethyl-1,3,2-dioxaborolan-2-yl)phenyl)propanoic acid.

### Step E: Synthesis of tert-butyl 3-(5-(2-chloro-4-(methoxymethoxy)-6-(4,4,5,5-tetramethyl-1,3,2-dioxaborolan-2-yl)phenethyl)-1,2,4-oxadiazol-3-yl)piperidine-1-carboxylate

3-(2-chloro-4-(methoxymethoxy)-6-(4,4,5,5-tetramethyl-1,3,2-dioxaborolan-2-yl)phenyl)propanoic acid is reacted with tert-butyl (Z)-3-(2-amino-2-(hydroxyimino)ethyl)piperidine-1-carboxylate using standard conditions to obtain tert-butyl 3-(5-(2-chloro-4-(methoxymethoxy)-6-(4,4,5,5-tetramethyl-1,3,2-dioxaborolan-2-yl)phenethyl)-1,2,4-oxadiazol-3-yl)piperidine-1-carboxylate.

### Step F: Synthesis of tert-butyl 3-((5-(2-chloro-6-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)-4-(methoxymethoxy)phenethyl)-1,2,4-oxadiazol-3-yl)methyl)piperidine-1-carboxylate

Tert-butyl 3-(5-(2-chloro-4-(methoxymethoxy)-6-(4,4,5,5-tetramethyl-1,3,2-dioxaborolan-2-yl)phenethyl)-1,2,4-oxadiazol-3-yl)piperidine-1-carboxylate is reacted with 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine under standard Suzuki coupling conditions to obtain tert-butyl 3-((5-(2-chloro-6-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)-4-(methoxymethoxy)phenethyl)-1,2,4-oxadiazol-3-yl)methyl)piperidine-1-carboxylate.

### Step G: Synthesis of (Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacycloheptaphan-1⁵-ol

Tert-butyl 3-((5-(2-chloro-6-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)-4-(methoxymethoxy)phenethyl)-1,2,4-oxadiazol-3-yl)methyl)piperidine-1-carboxylate is deprotected under standard conditions and is further subjected to standard SNAr conditions to obtain (Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacycloheptaphan-1⁵-ol.

### Step H: Synthesis of 3-chloro-4-(2-(3-((1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)methyl)-1,2,4-oxadiazol-5-yl)ethyl)-5-(4,4,5,5-tetramethyl-1,3,2-dioxaborolan-2-yl)phenol

Tert-butyl 3-(5-(2-chloro-4-(methoxymethoxy)-6-(4,4,5,5-tetramethyl-1,3,2-dioxaborolan-2-yl)phenethyl)-1,2,4-oxadiazol-3-yl)piperidine-1-carboxylate is deprotected under standard conditions and is further subjected to standard S_{N}Ar conditions with 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine to obtain 3-chloro-4-(2-(3-((1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)methyl)-1,2,4-oxadiazol-5-yl)ethyl)-5-(4,4,5,5-tetramethyl-1,3,2-dioxaborolan-2-yl)phenol.

### Step I: Synthesis of (Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacycloheptaphan-1⁵-ol

3-chloro-4-(2-(3-((1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)methyl)-1,2,4-oxadiazol-5-yl)ethyl)-5-(4,4,5,5-tetramethyl-1,3,2-dioxaborolan-2-yl)phenol is reacted under standard Suzuki coupling conditions to obtain (Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacycloheptaphan-1⁵-ol.

### Example 8. (Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacyclooctaphan-1⁵-ol (Compound 105a)

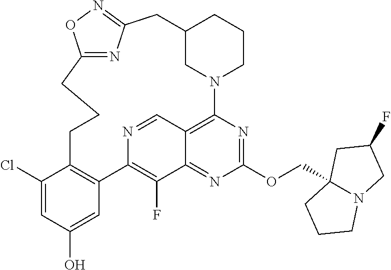 [SMILES: Oc1cc(Cl)c2c(c1)-c1ncc3c(nc(OC[C@@]45CCCN4C[C@H](F)C5)nc3c1F)N1CCCC(Cc3noc(n3)CCC2)C1]

(Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacyclooctaphan-1⁵-ol is prepared in an analogous manner to Example 7 by using (2-carboxyethyl)triphenylphosphonium bromide in Step A.

### Example 9. (Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacyclononaphan-1⁵-ol (Compound 106a)

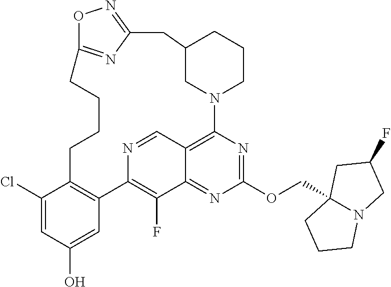 [SMILES: Oc1cc(Cl)c2c(c1)-c1ncc3c(nc(OC[C@@]45CCCN4C[C@H](F)C5)nc3c1F)N1CCCC(Cc3noc(n3)CCCC2)C1]

(Z)-1³-chloro-2⁸-fluoro-2²-(((2R,7aS)-2-fluorotetrahydro-1H-pyrrolizin-7a(5H)-yl)methoxy)-5(3,5)-oxadiazola-2(7,4)-pyrido[4,3-d]pyrimidina-3(1,3)-piperidina-1(1,2)-benzenacyclononaphan-1⁵-ol is prepared in an analogous manner to Example 7 by using (3-carboxypropyl)triphenylphosphonium bromide, respectively, in Step A.

### Example 11. (3³-chloro-2⁸-fluoro-2²-(((2R,7aS)-2-fluorotetrahydro-1H-pyrrolizin-7a(5H)-yl)methoxy)-3⁵-hydroxy-8-aza-2(4,7)-pyrido[4,3-d]pyrimidina-1(1,3)-piperidina-3(1,2)-benzenacyclodecaphan-7-one (Compound 114a)

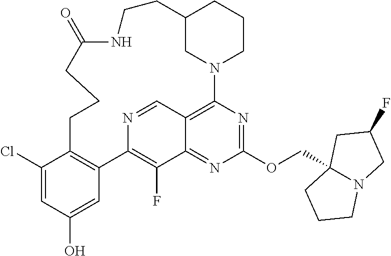 [SMILES: O=C1CCCc2c(Cl)cc(O)cc2-c2ncc3c(nc(OC[C@@]45CCCN4C[C@H](F)C5)nc3c2F)N2CCCC(CCN1)C2]

(3³-chloro-2⁸-fluoro-2²-(((2R,7aS)-2-fluorotetrahydro-1H-pyrrolizin-7a(5H)-yl)methoxy)-3⁵-hydroxy-8-aza-2(4,7)-pyrido[4,3-d]pyrimidina-1(1,3)-piperidina-3(1,2)-benzenacyclodecaphan-7-one is prepared in an analogous manner to Example 10 by using 4-(2-chloro-4-(methoxymethoxy)-6-(4,4,5,5-tetramethyl-1,3,2-dioxaborolan-2-yl)phenyl)butanoic acid in Step C.

### Example 12. (3³-chloro-2⁸-fluoro-2²-(((2R,7aS)-2-fluorotetrahydro-1H-pyrrolizin-7a(5H)-yl)methoxy)-3⁵-hydroxy-9-aza-2(4,7)-pyrido[4,3-d]pyrimidina-1(1,3)-piperidina-3(1,2)-benzenacycloundecaphan-8-one) (Compound 115a)

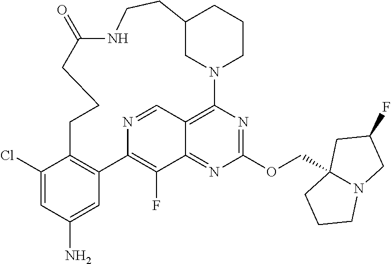 [SMILES: Nc1cc(Cl)c2c(c1)-c1ncc3c(nc(OC[C@@]45CCCN4C[C@H](F)C5)nc3c1F)N1CCCC(CCNC(=O)CCC2)C1]

(3³-chloro-2⁸-fluoro-2²-(((2R,7aS)-2-fluorotetrahydro-1H-pyrrolizin-7a(5H)-yl)methoxy)-3⁵-hydroxy-9-aza-2(4,7)-pyrido[4,3-d]pyrimidina-1(1,3)-piperidina-3(1,2)-benzenacycloundecaphan-8-one) is prepared in an analogous manner to Example 10 by using 5-(2-chloro-4-(methoxymethoxy)-6-(4,4,5,5-tetramethyl-1,3,2-dioxaborolan-2-yl)phenyl)pentanoic acid in Step C.

### Example 13. Synthesis of (Z)-2⁸,3⁷-difluoro-2²-(((2R,7aS)-2-fluorotetrahydro-1H-pyrrolizin-7a(5H)-yl)methoxy)-4¹H-2(4,7)-pyrido[4,3-d]pyrimidina-1(1,3)-piperidina-4(4,1)-triazola-3(1,8)-naphthalenacyclooctaphan-3³-ol (Compound 125a)

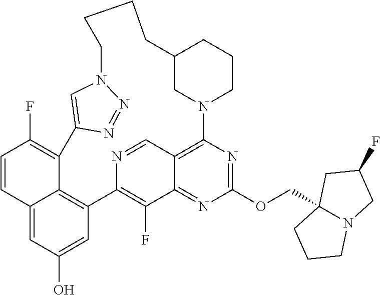 [SMILES: Oc1cc2c3c(c(F)ccc3c1)-c1cn(nn1)CCCCC1CCCN(C1)c1nc(OC[C@@]34CCCN3C[C@H](F)C4)nc3c(F)c-2ncc13]

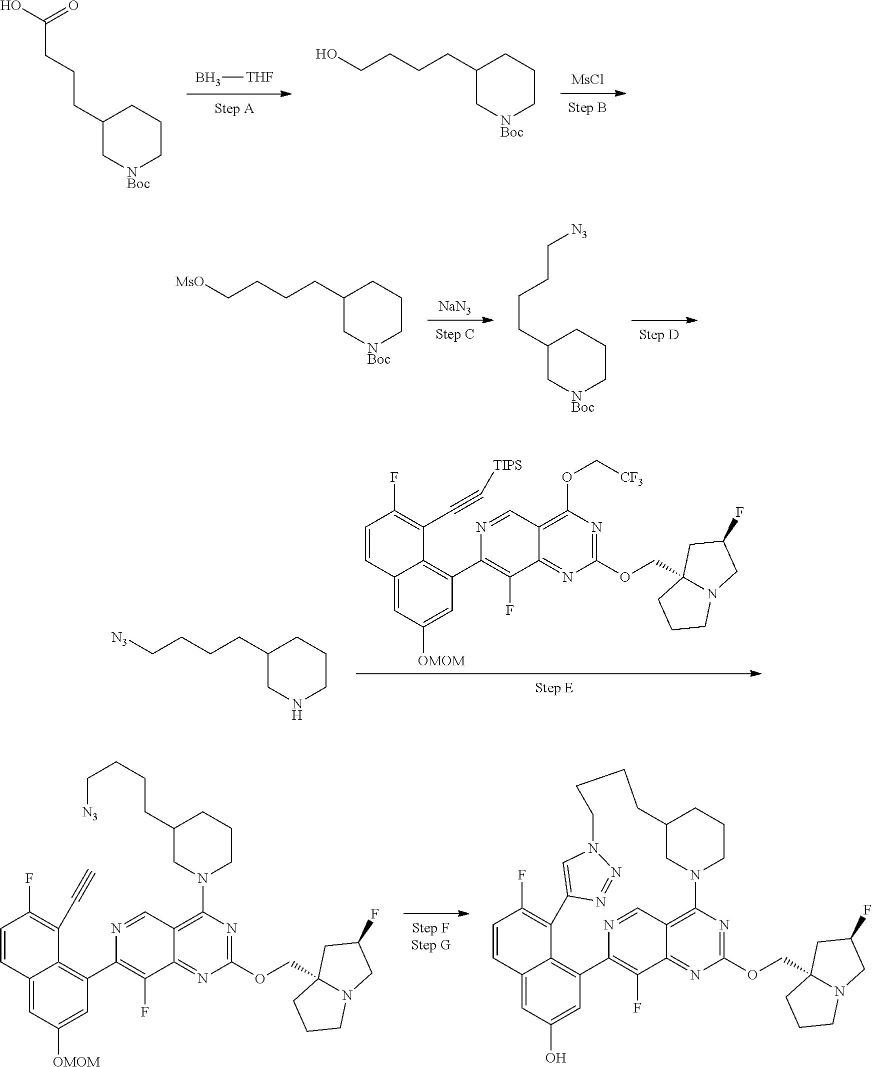

### Step A: Synthesis of tert-butyl 3-(4-hydroxybutyl)piperidine-1-carboxylate

A solution of 4-(1-(tert-butoxycarbonyl)piperidin-3-yl)butanoic acid (1.00 g, 1 equiv., 3.69 mmol) in THF (10 mL) was treated dropwise with borane tetrahydrofuran complex (317 mg, 3.69 mL, 1 equiv., 3.69 mmol). The reaction mixture was stirred at room temperature for 16 hours. Borane tetrahydrofuran complex (317 mg, 3.69 mL, 1 equiv., 3.69 mmol) was then added and the reaction mixture was stirred for 24 hours. The reaction mixture was concentrated under reduced pressure to afford tert-butyl 3-(4-hydroxybutyl)piperidine-1-carboxylate (1.028 g, 3.994 mmol, 108%) that was used without further purification.

MS (ESI): m/z (+Na)=280.3

### Step B: Synthesis of tert-butyl 3-(4-((methylsulfonyl)oxy)butyl)piperidine-1-carboxylate

A solution of tert-butyl 3-(4-hydroxybutyl)piperidine-1-carboxylate (1.00 g, 1 equiv., 3.89 mmol) and DIPEA (1.51 g, 2.03 mL, 3 equiv., 11.7 mmol) in DCM (15 mL) was treated with MsCl (534 mg, 363 μL, 1.2 equiv., 4.66 mmol) in one portion. The reaction mixture was stirred at room temperature for 3 days. DIPEA (0.755 g, 1.02 mL, 1.5 equiv., 5.85 mmol) and MsCl (267 mg, 182 μL, 0.6 equiv., 2.33 mmol) was added and the reaction mixture was stirred at room temperature for 3 hours. The reaction mixture was diluted with DCM and was washed with water and brine, dried (MgSO₄), filtered, and concentrated to afford tert-butyl 3-(4-((methylsulfonyl)oxy)butyl)piperidine-1-carboxylate (1.03 g, 78.9%).

MS (ESI): m/z (+Na)=358.3

### Step C: Synthesis of tert-butyl 3-(4-azidobutyl)piperidine-1-carboxylate

Tert-butyl 3-(4-((methylsulfonyl)oxy)butyl)piperidine-1-carboxylate is reacted with sodium azide under standard conditions to obtain tert-butyl 3-(4-azidobutyl)piperidine-1-carboxylate.

### Step D: Synthesis of 3-(4-azidobutyl)piperidine

Tert-butyl 3-(4-azidobutyl)piperidine-1-carboxylate is deprotected under standard conditions to obtain 3-(4-azidobutyl)piperidine.

### Step E: Synthesis of 4-(3-(4-azidobutyl)piperidin-1-yl)-7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidine

3-(4-azidobutyl)piperidine is reacted with 8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine under standard SNAr conditions to obtain 4-(3-(4-azidobutyl)piperidin-1-yl)-7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidine.

### Step F: Synthesis of (Z)-2⁸,3⁷-difluoro-2²-(((2R,7aS)-2-fluorotetrahydro-1H-pyrrolizin-7a(5H)-yl)methoxy)-3³-(methoxymethoxy)-4¹H-2(4,7)-pyrido[4,3-d]pyrimidina-1(1,3)-piperidina-4(4,1)-triazola-3(1,8)-naphthalenacyclooctaphane

4-(3-(4-azidobutyl)piperidin-1-yl)-7-(8-ethynyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidine is cyclized under standard conditions to obtain (Z)-2⁸,3⁷-difluoro-2²-(((2R,7aS)-2-fluorotetrahydro-1H-pyrrolizin-7a(5H)-yl)methoxy)-3³-(methoxymethoxy)-4¹H-2(4,7)-pyrido[4,3-d]pyrimidina-1(1,3)-piperidina-4(4,1)-triazola-3(1,8)-naphthalenacyclooctaphane.

### Step G: Synthesis of (Z)-2⁸,3⁷-difluoro-2²-(((2R,7aS)-2-fluorotetrahydro-1H-pyrrolizin-7a(5H)-yl)methoxy)-4¹H-2(4,7)-pyrido[4,3-d]pyrimidina-1(1,3)-piperidina-4(4,1)-triazola-3(1,8)-naphthalenacyclooctaphan-3³-ol

(Z)-2⁸,3⁷-difluoro-2²-(((2R,7aS)-2-fluorotetrahydro-1H-pyrrolizin-7a(5H)-yl)methoxy)-3³-(methoxymethoxy)-4¹H-2(4,7)-pyrido[4,3-d]pyrimidina-1(1,3)-piperidina-4(4,1)-triazola-3(1,8)-naphthalenacyclooctaphane is deprotected under standard conditions to obtain (Z)-2⁸,3⁷-difluoro-2²-(((2R,7aS)-2-fluorotetrahydro-1H-pyrrolizin-7a(5H)-yl)methoxy)-4¹H-2(4,7)-pyrido[4,3-d]pyrimidina-1(1,3)-piperidina-4(4,1)-triazola-3(1,8)-naphthalenacyclooctaphan-3³-ol.

### Example 14. Synthesis of (Z)-1³-chloro-2⁸-fluoro-2²-(((2R,7aS)-2-fluorotetrahydro-1H-pyrrolizin-7a(5H)-yl)methoxy)-5¹H-2(7,4)-pyrido[4,3-d]pyrimidina-3(1,3)-piperidina-5(4,1)-triazola-1(1,2)-benzenacyclooctaphan-1⁵-ol (Compound 121a)

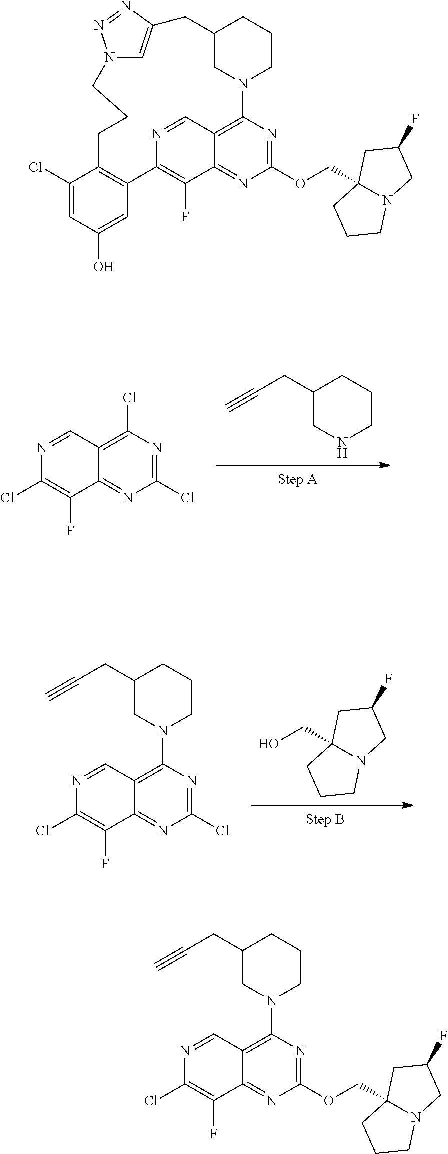

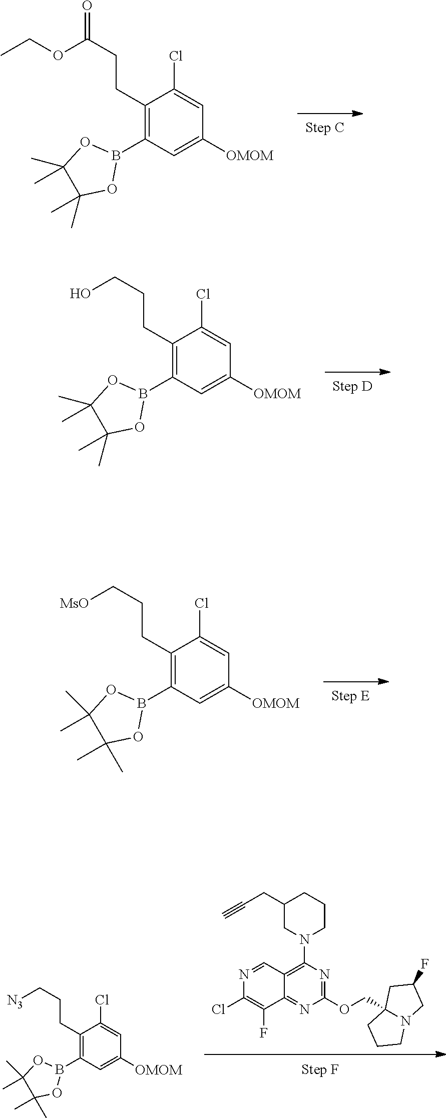

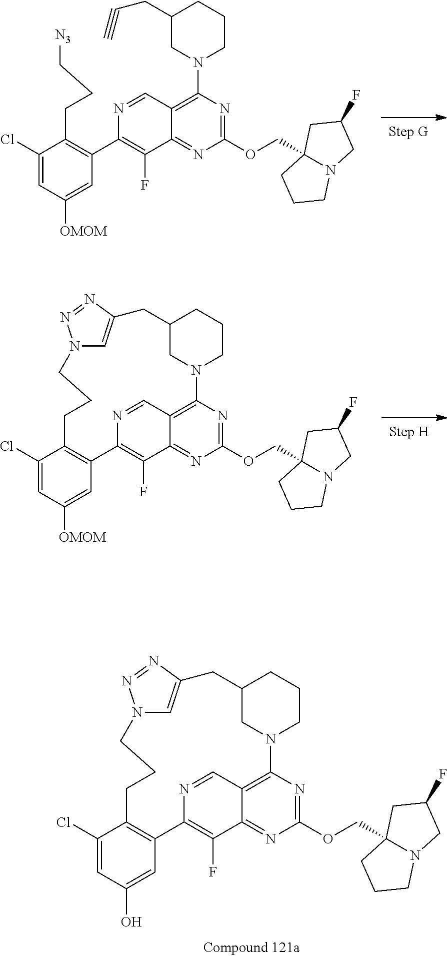

### Step A: Synthesis of 2,7-dichloro-8-fluoro-4-(3-(prop-2-yn-1-yl)piperidin-1-yl)pyrido[4,3-d]pyrimidine

2,4,7-trichloro-8-fluoropyrido[4,3-d]pyrimidine is reacted with 3-(prop-2-yn-1-yl)piperidine under standard SNAr conditions to obtain 2,7-dichloro-8-fluoro-4-(3-(prop-2-yn-1-yl)piperidin-1-yl)pyrido[4,3-d]pyrimidine.

### Step B: Synthesis of 7-chloro-8-fluoro-2-(((2R,7aS)-2-fluorotetrahydro-1H-pyrrolizin-7a(5H)-yl)methoxy)-4-(3-(prop-2-yn-1-yl)piperidin-1-yl)pyrido[4,3-d]pyrimidine

2,7-dichloro-8-fluoro-4-(3-(prop-2-yn-1-yl)piperidin-1-yl)pyrido[4,3-d]pyrimidine is reacted with ((2R,7aS)-2-fluorotetrahydro-1H-pyrrolizin-7a(5H)-yl)methanol under standard SNAr conditions to obtain 7-chloro-8-fluoro-2-(((2R,7aS)-2-fluorotetrahydro-1H-pyrrolizin-7a(5H)-yl)methoxy)-4-(3-(prop-2-yn-1-yl)piperidin-1-yl)pyrido[4,3-d]pyrimidine.

### Step C: Synthesis of 3-(2-chloro-4-(methoxymethoxy)-6-(4,4,5,5-tetramethyl-1,3,2-dioxaborolan-2-yl)phenyl)propan-1-ol

Ethyl 3-(2-chloro-4-(methoxymethoxy)-6-(4,4,5,5-tetramethyl-1,3,2-dioxaborolan-2-yl)phenyl)propanoate is reduced under standard conditions to obtain 3-(2-chloro-4-(methoxymethoxy)-6-(4,4,5,5-tetramethyl-1,3,2-dioxaborolan-2-yl)phenyl)propan-1-ol.

### Step D: Synthesis of 3-(2-chloro-4-(methoxymethoxy)-6-(4,4,5,5-tetramethyl-1,3,2-dioxaborolan-2-yl)phenyl)propyl methanesulfonate

3-(2-chloro-4-(methoxymethoxy)-6-(4,4,5,5-tetramethyl-1,3,2-dioxaborolan-2-yl)phenyl)propan-1-ol is reacted with MsCl under standard conditions to obtain 3-(2-chloro-4-(methoxymethoxy)-6-(4,4,5,5-tetramethyl-1,3,2-dioxaborolan-2-yl)phenyl)propyl methanesulfonate.

### Step E: Synthesis of 2-(2-(3-azidopropyl)-3-chloro-5-(methoxymethoxy)phenyl)-4,4,5,5-tetramethyl-1,3,2-dioxaborolane

3-(2-chloro-4-(methoxymethoxy)-6-(4,4,5,5-tetramethyl-1,3,2-dioxaborolan-2-yl)phenyl)propyl methanesulfonate is reacted with sodium azide under standard conditions to obtain 2-(2-(3-azidopropyl)-3-chloro-5-(methoxymethoxy)phenyl)-4,4,5,5-tetramethyl-1,3,2-dioxaborolane.

### Step F: Synthesis of 7-(2-(3-azidopropyl)-3-chloro-5-(methoxymethoxy)phenyl)-8-fluoro-2-(((2R,7aS)-2-fluorotetrahydro-1H-pyrrolizin-7a(5H)-yl)methoxy)-4-(3-(prop-2-yn-1-yl)piperidin-1-yl)pyrido[4,3-d]pyrimidine

2-(2-(3-azidopropyl)-3-chloro-5-(methoxymethoxy)phenyl)-4,4,5,5-tetramethyl-1,3,2-dioxaborolane is reacted with 7-chloro-8-fluoro-2-(((2R,7aS)-2-fluorotetrahydro-1H-pyrrolizin-7a(5H)-yl)methoxy)-4-(3-(prop-2-yn-1-yl)piperidin-1-yl)pyrido[4,3-d]pyrimidine under standard Suzuki coupling conditions to obtain 7-(2-(3-azidopropyl)-3-chloro-5-(methoxymethoxy)phenyl)-8-fluoro-2-(((2R,7aS)-2-fluorotetrahydro-1H-pyrrolizin-7a(5H)-yl)methoxy)-4-(3-(prop-2-yn-1-yl)piperidin-1-yl)pyrido[4,3-d]pyrimidine.

### Step G: Synthesis of (Z)-1³-chloro-2⁸-fluoro-2²-(((2R,7aS)-2-fluorotetrahydro-1H-pyrrolizin-7a(5H)-yl)methoxy)-1⁵-(methoxymethoxy)-5¹H-2(7,4)-pyrido[4,3-d]pyrimidina-3(1,3)-piperidina-5(4,1)-triazola-1(1,2)-benzenacyclooctaphane

7-(2-(3-azidopropyl)-3-chloro-5-(methoxymethoxy)phenyl)-8-fluoro-2-(((2R,7aS)-2-fluorotetrahydro-1H-pyrrolizin-7a(5H)-yl)methoxy)-4-(3-(prop-2-yn-1-yl)piperidin-1-yl)pyrido[4,3-d]pyrimidine is cyclized under standard conditions to obtain (Z)-1³-chloro-2⁸-fluoro-2²-(((2R,7aS)-2-fluorotetrahydro-1H-pyrrolizin-7a(5H)-yl)methoxy)-15-(methoxymethoxy)-5¹H-2(7,4)-pyrido[4,3-d]pyrimidina-3(1,3)-piperidina-5(4,1)-triazola-1(1,2)-benzenacyclooctaphane.

### Step H: Synthesis of (Z)-1³-chloro-2⁸-fluoro-2²-(((2R,7aS)-2-fluorotetrahydro-1H-pyrrolizin-7a(5H)-yl)methoxy)-5¹H-2(7,4)-pyrido[4,3-d]pyrimidina-3(1,3)-piperidina-5(4,1)-triazola-1(1,2)-benzenacyclooctaphan-1⁵-ol

(Z)-1³-chloro-2⁸-fluoro-2²-(((2R,7aS)-2-fluorotetrahydro-1H-pyrrolizin-7a(5H)-yl)methoxy)-1⁵-(methoxymethoxy)-5¹H-2(7,4)-pyrido[4,3-d]pyrimidina-3(1,3)-piperidina-5(4,1)-triazola-1(1,2)-benzenacyclooctaphane is deprotected under standard conditions to obtain (Z)-1³-chloro-2⁸-fluoro-2²-(((2R,7aS)-2-fluorotetrahydro-1H-pyrrolizin-7a(5H)-yl)methoxy)-5¹H-2(7,4)-pyrido[4,3-d]pyrimidina-3(1,3)-piperidina-5(4,1)-triazola-1(1,2)-benzenacyclooctaphan-1⁵-ol.

### Example 15. (Z)-1³-chloro-2⁸-fluoro-2²-(((2R,7aS)-2-fluorotetrahydro-1H-pyrrolizin-7a(5H)-yl)methoxy)-5¹H-2(7,4)-pyrido[4,3-d]pyrimidina-3(1,3)-piperidina-5(4,1)-triazola-1(1,2)-benzenacyclononaphan-1⁵-ol (Compound 122a)

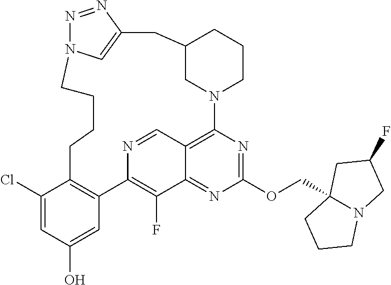 [SMILES: Oc1cc(Cl)c2c(c1)-c1ncc3c(nc(OC[C@@]45CCCN4C[C@H](F)C5)nc3c1F)N1CCCC(Cc3cn(nn3)CCCC2)C1]

(Z)-1³-chloro-2⁸-fluoro-2²-(((2R,7aS)-2-fluorotetrahydro-1H-pyrrolizin-7a(5H)-yl)methoxy)-5¹H-2(7,4)-pyrido[4,3-d]pyrimidina-3(1,3)-piperidina-5(4,1)-triazola-1(1,2)-benzenacyclononaphan-1⁵-ol is prepared in an analogous manner to Example 14 by using 4-(2-chloro-4-(methoxymethoxy)-6-(4,4,5,5-tetramethyl-1,3,2-dioxaborolan-2-yl)phenyl)butanoic acid in Step C.

### Example 16. Synthesis of (Z)-1³-chloro-2′-fluoro-2²-(((2R,7aS)-2-fluorotetrahydro-1H-pyrrolizin-7a(5H)-yl)methoxy)-5¹H-2(7,4)-pyrido[4,3-d]pyrimidina-3(1,3)-piperidina-5(1,4)-triazola-1(1,2)-benzenacyclooctaphan-1⁵-ol (Compound 123a)

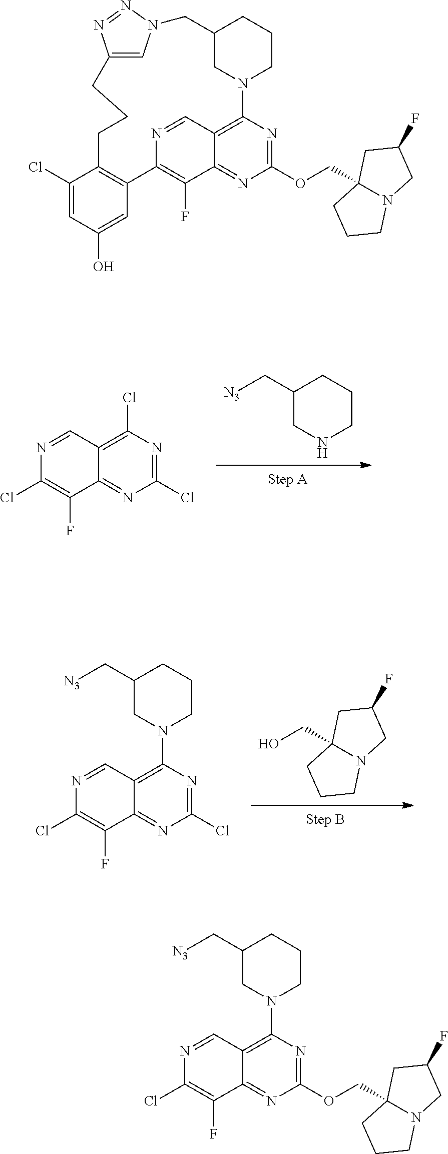 [SMILES: Oc1cc(Cl)c2c(c1)-c1ncc3c(nc(OC[C@@]45CCCN4C[C@H](F)C5)nc3c1F)N1CCCC(C1)Cn1cc(nn1)CCC2 ; Fc1c(Cl)ncc2c(Cl)nc(Cl)nc12 ; [N-]=[N+]=NCC1CCCNC1 ; [N-]=[N+]=NCC1CCCN(c2nc(Cl)nc3c(F)c(Cl)ncc23)C1 ; OC[C@@]12CCCN1C[C@H](F)C2 ; [N-]=[N+]=NCC1CCCN(c2nc(OC[C@@]34CCCN3C[C@H](F)C4)nc3c(F)c(Cl)ncc23)C1]

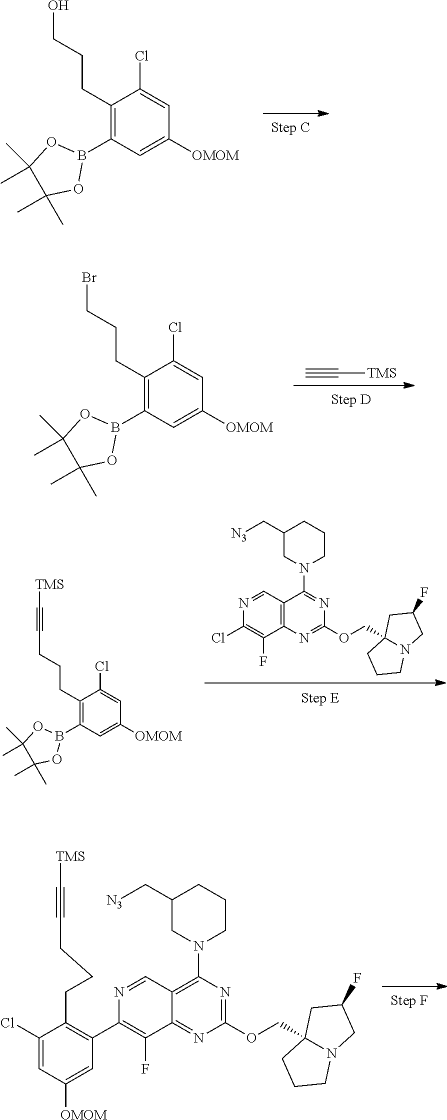

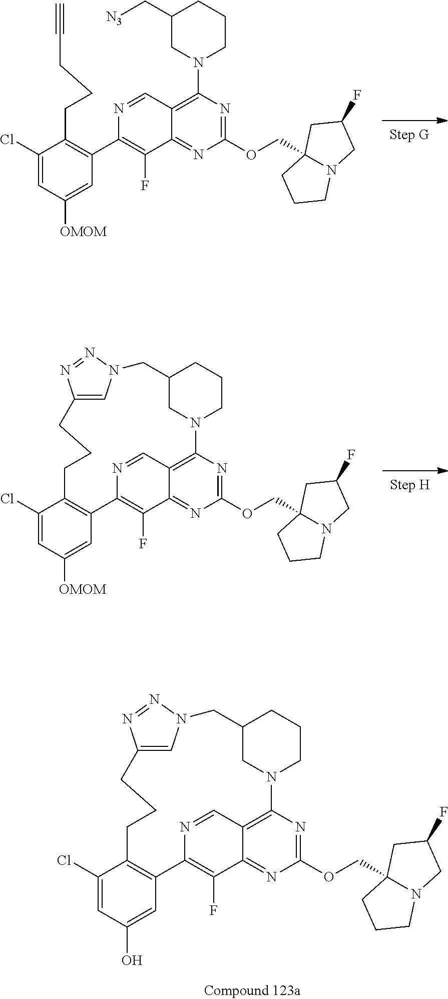

### Step A: Synthesis of 4-(3-(azidomethyl)piperidin-1-yl)-2,7-dichloro-8-fluoropyrido[4,3-d]pyrimidine

2,4,7-trichloro-8-fluoropyrido[4,3-d]pyrimidine is reacted with 3-(azidomethyl)piperidine under standard S_{N}Ar conditions to obtain 4-(3-(azidomethyl)piperidin-1-yl)-2,7-dichloro-8-fluoropyrido[4,3-d]pyrimidine.

### Step B: Synthesis of 4-(3-(azidomethyl)piperidin-1-yl)-7-chloro-8-fluoro-2-(((2R,7aS)-2-fluorotetrahydro-1H-pyrrolizin-7a(5H)-yl)methoxy)pyrido[4,3-d]pyrimidine

4-(3-(azidomethyl)piperidin-1-yl)-2,7-dichloro-8-fluoropyrido[4,3-d]pyrimidine is reacted with ((2R,7aS)-2-fluorotetrahydro-1H-pyrrolizin-7a(5H)-yl)methanol under standard SNAr conditions to obtain 4-(3-(azidomethyl)piperidin-1-yl)-7-chloro-8-fluoro-2-(((2R,7aS)-2-fluorotetrahydro-1H-pyrrolizin-7a(5H)-yl)methoxy)pyrido[4,3-d]pyrimidine.

### Step C: Synthesis of 2-(2-(3-bromopropyl)-3-chloro-5-(methoxymethoxy)phenyl)-4,4,5,5-tetramethyl-1,3,2-dioxaborolane

3-(2-chloro-4-(methoxymethoxy)-6-(4,4,5,5-tetramethyl-1,3,2-dioxaborolan-2-yl)phenyl)propan-1-ol is brominated under standard conditions to obtain 2-(2-(3-bromopropyl)-3-chloro-5-(methoxymethoxy)phenyl)-4,4,5,5-tetramethyl-1,3,2-dioxaborolane.

### Step D: Synthesis of (5-(2-chloro-4-(methoxymethoxy)-6-(4,4,5,5-tetramethyl-1,3,2-dioxaborolan-2-yl)phenyl)pent-1-yn-1-yl)trimethylsilane

2-(2-(3-bromopropyl)-3-chloro-5-(methoxymethoxy)phenyl)-4,4,5,5-tetramethyl-1,3,2-dioxaborolane is reacted with ethynyltrimethylsilane under standard conditions to obtain (5-(2-chloro-4-(methoxymethoxy)-6-(4,4,5,5-tetramethyl-1,3,2-dioxaborolan-2-yl)phenyl)pent-1-yn-1-yl)trimethylsilane.

### Step E: Synthesis of 4-(3-(azidomethyl)piperidin-1-yl)-7-(3-chloro-5-(methoxymethoxy)-2-(5-(trimethylsilyl)pent-4-yn-1-yl)phenyl)-8-fluoro-2-(((2R,7aS)-2-fluorotetrahydro-1H-pyrrolizin-7a(5H)-yl)methoxy)pyrido[4,3-d]pyrimidine

(5-(2-chloro-4-(methoxymethoxy)-6-(4,4,5,5-tetramethyl-1,3,2-dioxaborolan-2-yl)phenyl)pent-1-yn-1-yl)trimethylsilane is reacted with 4-(3-(azidomethyl)piperidin-1-yl)-7-chloro-8-fluoro-2-(((2R,7aS)-2-fluorotetrahydro-1H-pyrrolizin-7a(5H)-yl)methoxy)pyrido[4,3-d]pyrimidine under standard Suzuki coupling conditions to obtain 4-(3-(azidomethyl)piperidin-1-yl)-7-(3-chloro-5-(methoxymethoxy)-2-(5-(trimethylsilyl)pent-4-yn-1-yl)phenyl)-8-fluoro-2-(((2R,7aS)-2-fluorotetrahydro-1H-pyrrolizin-7a(5H)-yl)methoxy)pyrido[4,3-d]pyrimidine.

### Step F: Synthesis of 4-(3-(azidomethyl)piperidin-1-yl)-7-(3-chloro-5-(methoxymethoxy)-2-(pent-4-yn-1-yl)phenyl)-8-fluoro-2-(((2R,7aS)-2-fluorotetrahydro-1H-pyrrolizin-7a(5H)-yl)methoxy)pyrido[4,3-d]pyrimidine

4-(3-(azidomethyl)piperidin-1-yl)-7-(3-chloro-5-(methoxymethoxy)-2-(5-(trimethylsilyl)pent-4-yn-1-yl)phenyl)-8-fluoro-2-(((2R,7aS)-2-fluorotetrahydro-1H-pyrrolizin-7a(5H)-yl)methoxy)pyrido[4,3-d]pyrimidine is deprotected under standard conditions to obtain 4-(3-(azidomethyl)piperidin-1-yl)-7-(3-chloro-5-(methoxymethoxy)-2-(pent-4-yn-1-yl)phenyl)-8-fluoro-2-(((2R,7aS)-2-fluorotetrahydro-1H-pyrrolizin-7a(5H)-yl)methoxy)pyrido[4,3-d]pyrimidine.

### Step G: Synthesis of (Z)-1³-chloro-2⁸-fluoro-2²-(((2R,7aS)-2-fluorotetrahydro-1H-pyrrolizin-7a(5H)-yl)methoxy)-1⁵-(methoxymethoxy)-5¹H-2(7,4)-pyrido[4,3-d]pyrimidina-3(1,3)-piperidina-5(1,4)-triazola-1(1,2)-benzenacyclooctaphane

4-(3-(azidomethyl)piperidin-1-yl)-7-(3-chloro-5-(methoxymethoxy)-2-(pent-4-yn-1-yl)phenyl)-8-fluoro-2-(((2R,7aS)-2-fluorotetrahydro-1H-pyrrolizin-7a(5H)-yl)methoxy)pyrido[4,3-d]pyrimidine is cyclized under standard conditions to obtain (Z)-13-chloro-2⁸-fluoro-2²-(((2R,7aS)-2-fluorotetrahydro-1H-pyrrolizin-7a(5H)-yl)methoxy)-15-(methoxymethoxy)-5¹H-2(7,4)-pyrido[4,3-d]pyrimidina-3(1,3)-piperidina-5(1,4)-triazola-1(1,2)-benzenacyclooctaphane.

### Step H: Synthesis of (Z)-1³-chloro-2⁸-fluoro-2²-(((2R,7aS)-2-fluorotetrahydro-1H-pyrrolizin-7a(5H)-yl)methoxy)-5¹H-2(7,4)-pyrido[4,3-d]pyrimidina-3(1,3)-piperidina-5(1,4)-triazola-1(1,2)-benzenacyclooctaphan-1⁵-ol

(Z)-1³-chloro-2⁸-fluoro-2²-(((2R,7aS)-2-fluorotetrahydro-1H-pyrrolizin-7a(5H)-yl)methoxy)-1⁵-(methoxymethoxy)-5¹H-2(7,4)-pyrido[4,3-d]pyrimidina-3(1,3)-piperidina-5(1,4)-triazola-1(1,2)-benzenacyclooctaphane was deprotected under standard conditions to obtain (Z)-1³-chloro-2⁸-fluoro-2²-(((2R,7aS)-2-fluorotetrahydro-1H-pyrrolizin-7a(5H)-yl)methoxy)-5¹H-2(7,4)-pyrido[4,3-d]pyrimidina-3(1,3)-piperidina-5(1,4)-triazola-1(1,2)-benzenacyclooctaphan-1⁵-ol.

### Example 17. (Z)-1³-chloro-2⁸-fluoro-2²-(((2R,7aS)-2-fluorotetrahydro-1H-pyrrolizin-7a(5H)-yl)methoxy)-5¹H-2(7,4)-pyrido[4,3-d]pyrimidina-3(1,3)-piperidina-5(1,4)-triazola-1(1,2)-benzenacyclononaphan-1⁵-ol (Compound 124a)

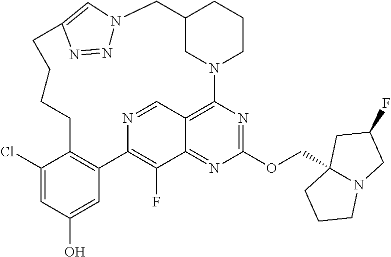 [SMILES: Oc1cc(Cl)c2c(c1)-c1ncc3c(nc(OC[C@@]45CCCN4C[C@H](F)C5)nc3c1F)N1CCCC(C1)Cn1cc(nn1)CCCC2]

(Z)-1³-chloro-2⁸-fluoro-2²-(((2R,7aS)-2-fluorotetrahydro-1H-pyrrolizin-7a(5H)-yl)methoxy)-5¹H-2(7,4)-pyrido[4,3-d]pyrimidina-3(1,3)-piperidina-5(1,4)-triazola-1(1,2)-benzenacyclononaphan-1⁵-ol was prepared in an analogous manner to Example 16 by using 4-(2-chloro-4-(methoxymethoxy)-6-(4,4,5,5-tetramethyl-1,3,2-dioxaborolan-2-yl)phenyl)butan-1-ol in Step C.

### Example 18. Synthesis of (Z)-1³-chloro-2⁸-fluoro-2²-(((2R,7aS)-2-fluorotetrahydro-1H-pyrrolizin-7a(5H)-yl)methoxy)-4¹H-2(7,4)-pyrido[4,3-d]pyrimidina-3(1,3)-piperidina-4(1,3)-triazola-1(1,2)-benzenacycloheptaphan-1⁵-ol (Compound 132a)

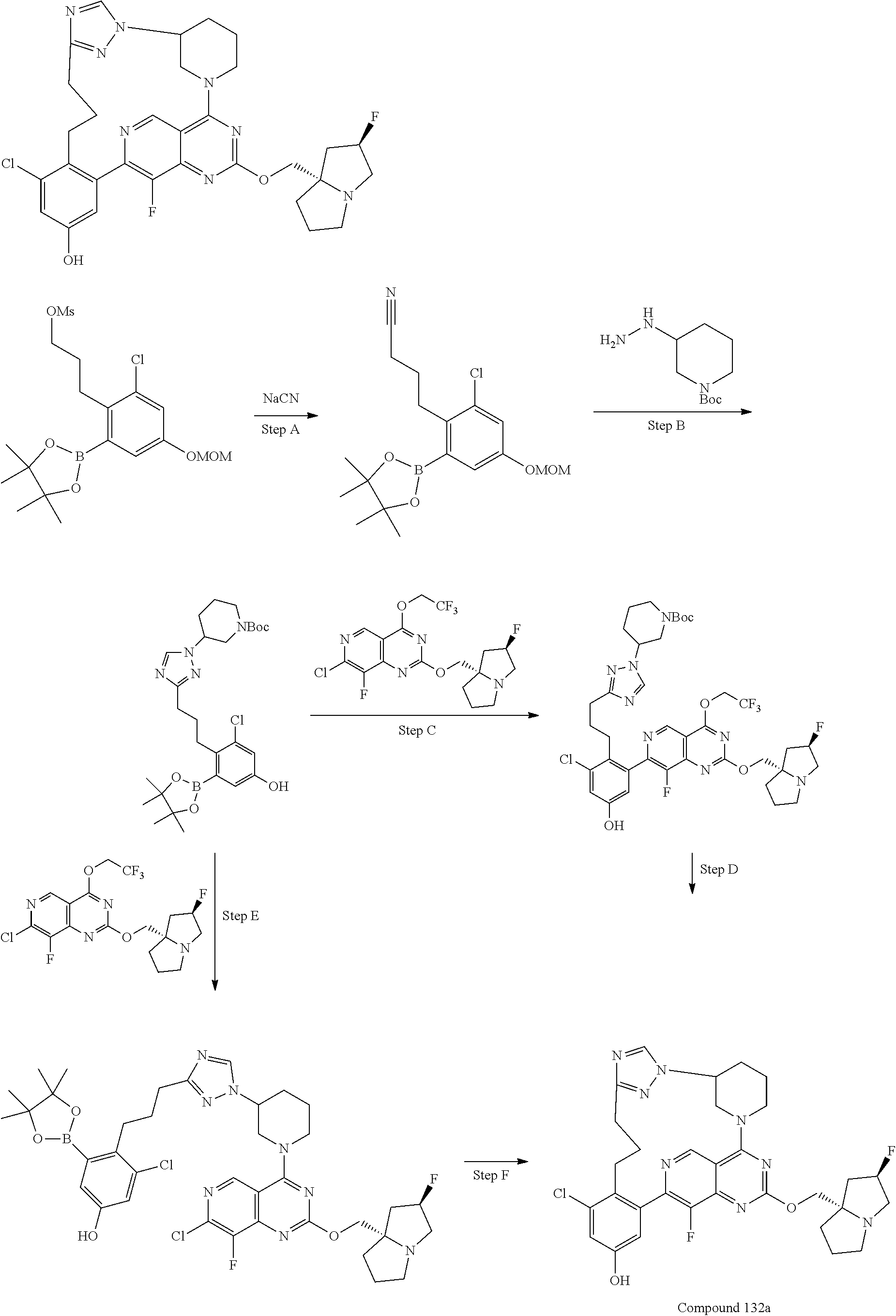

### Step A: Synthesis of 4-(2-chloro-4-(methoxymethoxy)-6-(4,4,5,5-tetramethyl-1,3,2-dioxaborolan-2-yl)phenyl)butanenitrile

3-(2-chloro-4-(methoxymethoxy)-6-(4,4,5,5-tetramethyl-1,3,2-dioxaborolan-2-yl)phenyl)propyl methanesulfonate is reacted with sodium cyanide under standard conditions to obtain 4-(2-chloro-4-(methoxymethoxy)-6-(4,4,5,5-tetramethyl-1,3,2-dioxaborolan-2-yl)phenyl)butanenitrile.

### Step B: Synthesis of tert-butyl 3-(3-(3-(2-chloro-4-hydroxy-6-(4,4,5,5-tetramethyl-1,3,2-dioxaborolan-2-yl)phenyl)propyl)-1H-1,2,4-triazol-1-yl)piperidine-1-carboxylate

4-(2-chloro-4-(methoxymethoxy)-6-(4,4,5,5-tetramethyl-1,3,2-dioxaborolan-2-yl)phenyl)butanenitrile is cyclized with tert-butyl 3-hydrazineylpiperidine-1-carboxylate under standard conditions to obtain tert-butyl 3-(3-(3-(2-chloro-4-hydroxy-6-(4,4,5,5-tetramethyl-1,3,2-dioxaborolan-2-yl)phenyl)propyl)-1H-1,2,4-triazol-1-yl)piperidine-1-carboxylate.

### Step C: Synthesis of tert-butyl 3-(3-(3-(2-chloro-6-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)-4-hydroxyphenyl)propyl)-1H-1,2,4-triazol-1-yl)piperidine-1-carboxylate

Tert-butyl 3-(3-(3-(2-chloro-4-hydroxy-6-(4,4,5,5-tetramethyl-1,3,2-dioxaborolan-2-yl)phenyl)propyl)-1H-1,2,4-triazol-1-yl)piperidine-1-carboxylate is reacted with 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine under Suzuki coupling conditions to obtain tert-butyl 3-(3-(3-(2-chloro-6-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)-4-hydroxyphenyl)propyl)-1H-1,2,4-triazol-1-yl)piperidine-1-carboxylate.

### Step D: Synthesis of (Z)-1³-chloro-2⁸-fluoro-2²-(((2R,7aS)-2-fluorotetrahydro-1H-pyrrolizin-7a(5H)-yl)methoxy)-4¹H-2(7,4)-pyrido[4,3-d]pyrimidina-3(1,3)-piperidina-4(1,3)-triazola-1(1,2)-benzenacycloheptaphan-1⁵-ol

Tert-butyl 3-(3-(3-(2-chloro-6-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)-4-hydroxyphenyl)propyl)-1H-1,2,4-triazol-1-yl)piperidine-1-carboxylate is deprotected under standard conditions and is further subjected to standard SNAr conditions to obtain (Z)-1³-chloro-2⁸-fluoro-2²-(((2R,7aS)-2-fluorotetrahydro-1H-pyrrolizin-7a(5H)-yl)methoxy)-4¹H-2(7,4)-pyrido[4,3-d]pyrimidina-3(1,3)-piperidina-4(1,3)-triazola-1(1,2)-benzenacycloheptaphan-1⁵-ol.

### Step E: Synthesis of 3-chloro-4-(3-(1-(1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)-1H-1,2,4-triazol-3-yl)propyl)-5-(4,4,5,5-tetramethyl-1,3,2-dioxaborolan-2-yl)phenol

Tert-butyl 3-(3-(3-(2-chloro-4-hydroxy-6-(4,4,5,5-tetramethyl-1,3,2-dioxaborolan-2-yl)phenyl)propyl)-1H-1,2,4-triazol-1-yl)piperidine-1-carboxylate is deprotected under standard conditions and is further subjected to standard SNAr conditions with 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine to obtain 3-chloro-4-(3-(1-(1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)-1H-1,2,4-triazol-3-yl)propyl)-5-(4,4,5,5-tetramethyl-1,3,2-dioxaborolan-2-yl)phenol.

### Step F: Synthesis of (Z)-1³-chloro-2⁸-fluoro-2²-(((2R,7aS)-2-fluorotetrahydro-1H-pyrrolizin-7a(5H)-yl)methoxy)-4¹H-2(7,4)-pyrido[4,3-d]pyrimidina-3(1,3)-piperidina-4(1,3)-triazola-1(1,2)-benzenacycloheptaphan-1⁵-ol

3-chloro-4-(3-(1-(1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)-1H-1,2,4-triazol-3-yl)propyl)-5-(4,4,5,5-tetramethyl-1,3,2-dioxaborolan-2-yl)phenol is reacted under standard Suzuki coupling conditions to obtain (Z)-1³-chloro-2⁸-fluoro-2²-(((2R,7aS)-2-fluorotetrahydro-1H-pyrrolizin-7a(5H)-yl)methoxy)-4¹H-2(7,4)-pyrido[4,3-d]pyrimidina-3(1,3)-piperidina-4(1,3)-triazola-1(1,2)-benzenacycloheptaphan-1⁵-ol.

### Example 19. (Z)-1³-chloro-2⁸-fluoro-2²-(((2R,7aS)-2-fluorotetrahydro-1H-pyrrolizin-7a(5H)-yl)methoxy)-4¹H-2(7,4)-pyrido[4,3-d]pyrimidina-3(1,3)-piperidina-4(1,3)-triazola-1(1,2)-benzenacyclooctaphan-1⁵-ol (Compound 133a)

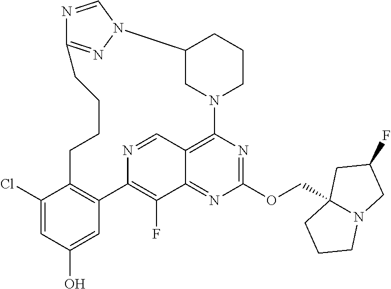 [SMILES: Oc1cc(Cl)c2c(c1)-c1ncc3c(nc(OC[C@@]45CCCN4C[C@H](F)C5)nc3c1F)N1CCCC(C1)n1cnc(n1)CCCC2]

(Z)-1³-chloro-2⁸-fluoro-2²-(((2R,7aS)-2-fluorotetrahydro-1H-pyrrolizin-7a(5H)-yl)methoxy)-4¹H-2(7,4)-pyrido[4,3-d]pyrimidina-3(1,3)-piperidina-4(1,3)-triazola-1(1,2)-benzenacyclooctaphan-1⁵-ol is prepared in an analogous manner to Example 18 by using 4-(2-chloro-4-(methoxymethoxy)-6-(4,4,5,5-tetramethyl-1,3,2-dioxaborolan-2-yl)phenyl)butyl methanesulfonate in Step A.

### Example 20. (Z)-1³-chloro-2⁸-fluoro-2²-(((2R,7aS)-2-fluorotetrahydro-1H-pyrrolizin-7a(5H)-yl)methoxy)-4¹H-2(7,4)-pyrido[4,3-d]pyrimidina-3(1,3)-piperidina-4(1,3)-triazola-1(1,2)-benzenacyclononaphan-1⁵-ol (Compound 134a)

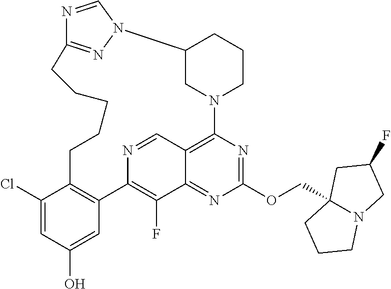 [SMILES: Oc1cc(Cl)c2c(c1)-c1ncc3c(nc(OC[C@@]45CCCN4C[C@H](F)C5)nc3c1F)N1CCCC(C1)n1cnc(n1)CCCCC2]

(Z)-1³-chloro-2⁸-fluoro-2²-(((2R,7aS)-2-fluorotetrahydro-1H-pyrrolizin-7a(5H)-yl)methoxy)-4¹H-2(7,4)-pyrido[4,3-d]pyrimidina-3(1,3)-piperidina-4(1,3)-triazola-1(1,2)-benzenacyclononaphan-1⁵-ol is prepared in an analogous manner to Example 18 by using 5-(2-chloro-4-(methoxymethoxy)-6-(4,4,5,5-tetramethyl-1,3,2-dioxaborolan-2-yl)phenyl)pentyl methanesulfonate, in Step A.

### Example B1. Surface Plasmon Resonance

Methods herein pertain to Cytiva (formerly Biacore) instrumentation and consumables and are applicable to other SPR-based systems. A Series S Streptavidin or Neutravidin chip is docked into a Biacore 8K. The instrument is then primed into an appropriate running buffer (for example, 20 mM HEPES, pH 7.4, 150 mM NaCl, 5 mM MgCl₂, 1 mM TCEP, 0.005% Tween-20, and 2% DMSO) supplemented with either 5 μM GDP or GMPPNP to match the nucleotide state of the KRas state being tested (GDP for KRas in the GDP-bound state; and GMPPNP for KRas in the GTP-bound state). Biotinylated-KRas loaded with the appropriate nucleotide state (e.g., GDP or GMPPNP) is then captured to between 500-2000 RU to provide adequate signal for small molecules of interest, typically in a range of 300-600 Da, as well as to accommodate a range of potencies, when appropriate. The remaining available biotin-binding sites are then blocked with Biocytin. Compounds are injected over both a reference surface (Streptavidin or Neutravidin alone) as well as the active surface (KRas coupled to Neutravidin or Streptavidin), and all curves are double-referenced before analysis (specifically, a signal subtraction of the reference flow cell from the active flow cell as well as a buffer subtraction). Where appropriate, a solvent correction is applied to all curves to account for differential bulk effects from DMSO on the reference and active surfaces. Data is then analyzed using the Biacore software fitting kinetics if resolvable, or fitting via a Langmuir isotherm model if no kinetics are resolvable, to report a binding affinity as a dissociation constant, K_{D}.

### Example B2. SOS1-Catalyzed Nucleotide Exchange Assay

KRas G12R and WT: Compounds were pre-dispensed using acoustic transfer technology into a black, low volume 384-well assay plate. A 10-point dose response of each compound was performed with a 30 μM top dose. Biotinylated KRas WT(1-169) or KRas G12R (1-169) loaded with GDP nucleotide was mixed with Streptavidin-Tb cryptate (Cisbio) in assay buffer (20 mM HEPES pH 7.4, 150 mM NaCl, 2 mM MgCl₂, and 0.005% CA680) to produce a 1.5× solution. 10 μL of the 1.5× KRas-cryptate solution was added to wells of a black, low-volume 384-well assay plate. The KRas/cryptate-compound mixture was incubated for 1 hour at room temperature. A 3× solution of SOS1 (564-1049) and EDA-GTP-DY-647P1 (Jena Bioscience) was prepared in assay buffer. 5 μL of the SOS1-labeled GTP solution was added to the wells to initiate the nucleotide exchange reaction. The final concentration of KRas G12R and SOS1 were 10 nM and 200 nM, respectively. The final concentration of KRas WT and SOS1 were 20 nM and 10 nM, respectively. The plate was allowed to incubate for 1 hr, then time resolved fluorescence was read on a PHERAstar plate reader equipped with a filter module with excitation=337 nm and emission 1=620 nm, emission 2=665 nm. The TR-FRET signal was calculated as the ratio of fluorescence intensity [emission 665 nm]/[emission 620 nm]. IC₅₀ values were calculated using a four-parameter, variable response sigmoidal dose response curve fit in PerkinElmer Signals VitroVivo.

KRas G12V [Method 1]: Biotinylated KRas G12V (aa 1-169) loaded with GDP nucleotide and Streptavidin-Europium (SA-EU, Columbia Biosciences) were preincubated in assay buffer (20 mM HEPES pH 7.4, 150 mM NaCl, 5 mM MgCl₂, 1 mM DTT, 0.01% Brij-35, 0.3 mg/mL BSA) on ice for 30 min. Separately SOS1 (aa 546-1049) and EDA-GTP-DY647P1 (Jena Bioscience) were preincubated in assay buffer on ice for 30 min. After pre-incubation, 5 μL buffer and 5 μL of KRas G12V-SA-Eu were added to each well of a white, low-volume, non-binding 384-well plate. Compounds were then added to respective wells with a Tecan D300e liquid dispenser. A 10-point concentration response curve for each compound was prepared with appropriate top concentration (1 μM, 10 μM, or 300 μM) and incubated for 30 min. 5 μL of SOS1-EDA-GTP-D467P1 was added to start the nucleotide exchange reaction. Final concentration in the assay were 5 nM KRas G12V, 50 nM SOS1 (aa 564-1049). The reaction was allowed to proceed for 60-90 minutes after which the plate was read on a PHERAstar FSX plate reader (exc: 337 nm, em 1: 665 nm, em 2: 620 nm). After normalization of the raw data (ratio of Signal₆₆₅ₙₘ/Signal₆₂₀ₙₘ) the data was fit to a four-parameter logistic curve in GraphPad Prism (v9.4.1).

KRas G12V [Method 2]: Compounds were pre-dispensed via Echo liquid handling (acoustic, touch-free) to generate assay ready plates (ARP). More precisely, a 10-point concentration response curve for each compound was prepared with appropriate top concentration (1 μM, 10 μM, 100 μM). Biotinylated KRas G12V (aa 1-169) loaded with GDP nucleotide and Streptavidin-Europium (SA-EU, Columbia Biosciences) were preincubated in assay buffer (20 mM HEPES pH 7.4, 150 mM NaCl, 5 mM MgCl₂, 1 mM DTT, 0.01% Brij-35, 0.3 mg/mL BSA) on ice for 30 min. Separately SOS1 (aa 546-1049) and EDA-GTP-DY647P1 (Jena Bioscience) were preincubated in assay buffer on ice for 30 min. After 30 minutes of incubation for the assay components, 5 μL buffer and 5 μL of KRas G12V-SA-Eu were added to each well of the ARP. After a further 30 min, 5 μL of SOS1-EDA-GTP-D467P1 was added to start the nucleotide exchange reaction. Final concentration in the assay were 5 nM KRas G12V, 50 nM SOS1 (aa 564-1049). The reaction was allowed to proceed for 60-90 minutes after which the plate was read on a plate reader. After normalization of the raw data, the data was fit to a four-parameter logistic curve.

The results are summarized in Table B1, below:

TABLE B1

| | KRas | KRas | KRas | | |
|---|---|---|---|---|---|
| | G12V IC₅₀ | G12V | G12V | KRas | KRas |
| Com- | (nM) | IC₅₀ (nM) | IC₅₀ (nM) | WT | G12R |
| pound | (Method | (Method | (Method | IC₅₀ | IC₅₀ |
| No. | 1) | 2, A) | 2, B) | (nM) | (nM) |
| 113a | ++ | | | ++ | + |
| 115a | ++++ | | | ++++ | ++++ |
| 115b | ++++ | | | ++ ++ | ++++ |
| 115c | ++++ | | | ++++ | ++++ |
| 118a | | | ++++ | | |
| 121a | | | | | |
| 125a | + | | | ++ | + |
| 135a | | ++++ | | | |
| 136a | | | ++++ | | |
| 140a | ++++ | | | | |
| 140b | | ++++ | ++++ | | |
| 140c | | | ++++ | | |
| 141a | ++++ | | | | |
| 143a | +++ | | | ++++ | +++ |
| 151a | ++++ | | | ++++ | ++++ |
| 152a | ++++ | | | ++++ | ++++ |
| 153a | +++ | | | +++ | +++ |
| 154a | | ++++ | | | |
| 155a | | ++ | | | |
| 156a | | ++++ | | | |
| 157a | | ++ | | | |
| 158a | | ++ | | | |
| 159a | | ++ | | | |
| 159b | | | ++ | | |
| 159c | | | + | | |
| 160a | | + | | | |
| 161a | | ++ | | | |
| 162a | | | +++ | | |
| 163a | | | ++ | | |
| 164a | | | ++ | | |
| 165a | | | ++++ | | |
| 166a | | | ++++ | | |
| 167a | | | ++++ | | |
| 170a | | | ++++ | | |
| 171a | | | +++ | | |
| 172a | | | ++++ | | |
| 173a | | | ++++ | | |
| 174a | | | +++ | | |
| 175a | | | ++++ | | |
| 176a | | | +++ | | |
| 177 | | | +++ | | |
| 178 | | | +++ | | |
| 179a | | | +++ | | |
| 180a | | | +++ | | |
| 181a | | | ++ | | |
| 182a | | | +++ | | |
| 183a | | | +++ | | |
| 184a | | | +++ | | |
| 185a | | | +++ | | |
| 186a | | | +++ | | |
| 187a | | | ++ | | |
| 188a | | | + | | |
| 189a | | +++ | +++ | | |
| 190a | | +++ | +++ | | |
| 191a | | +++ | ++++ | | |
| 192a | | +++ | +++ | | |
| 193a | | +++ | +++ | | |
| 194a | | ++++ | ++++ | | |
| 195a | | ++++ | ++++ | | |
| 196a | | | ++++ | | |
| 197a | | | ++++ | | |
| 197b | | | ++++ | | |
| 198a | | | ++++ | | |
| 199a | | | ++++ | | |
| 200a | | | ++++ | | |
| 201a | | | +++ | | |
| 202a | | | ++++ | | |
| 202b | | | ++++ | | |
| 203a | | | ++++ | | |

Notes:
“++++”: IC₅₀ ≤ 100 nM;
“+++”: 100 nM < IC₅₀ ≤ 1000 nM;
“++”: 1000 nM < IC₅₀ ≤ 10,000 nM; and
“+”: 10,000 nM < IC₅₀ ≤ 30,000 nM

### Example B3. KRas-cRAF Protein-Protein Interaction (PPI) Assay

Compounds are pre-dispensed using acoustic transfer technology into a black, low volume 384-well assay plate. A 10-point dose response of each compound is performed with a 30 μM top dose. Biotinylated KRas protein (e.g., KRas G12R(1-169)) is loaded with GppNHp (i.e., GMPPNP) nucleotide and GST-cRAF(1-149) are diluted to 90 nM and 30 nM, respectively, in assay buffer (20 mM HEPES pH 7.4, 150 mM NaCl, 5 mM MgCl₂, and 0.005%0 CA680). 5 μL of KRas protein is added to wells of a black, low-volume 384-well assay plate. The KRas-compound mixture is incubated for 30 minutes at room temperature. 5 μL of GST-cRAF protein is added to the KRas-compound mixture and incubated for 30 minutes at room temperature. 100× stocks of Tb cryptate-labeled anti-GST antibody (Anti-GST-Tb) (Cisbio) and Streptavidin-XL655 (Cisbio) are used to make a 3× detection mixture in a total volume of 5 μL of assay buffer. The detection mixture is added to the assay wells and incubated an additional 1 hours at room temperature. Time resolved fluorescence is read on a PHERAstar plate reader equipped with a filter module with excitation=337 nm and emission 1=620 nm, emission 2=665 nm. The TR-FRET signal is calculated as the ratio of fluorescence intensity [emission 665 nm]/[excitation 337 nm]. IC₅₀ values are calculated using a four-parameter, variable response sigmoidal dose response curve fit in PerkinElmer Signals VitroVivo.

### Example B4. KRas-cRAF Protein-Protein Interaction (PPI) Assay

Compounds are pre-dispensed via Echo liquid handling (acoustic, touch-free) to generate assay ready plates (ARP). A 10-point dose response of each compound is performed with a 30 μM top dose. Biotinylated KRas G12V (aa 1-169) loaded with GMPPNP nucleotide and Streptavidin-Europium (SA-EU, Columbia Biosciences) are preincubated in assay buffer (20 mM HEPES pH 7.4, 150 mM NaCl, 5 mM MgCl₂, 1 mM DTT, 0.01% Brij-35, 0.3 mg/ml BSA) on ice for 30 min. Separately GST-tagged cRAF(1-149) and anti-GST-APC antibody (Columbia Biosciences) are preincubated in assay buffer on ice for 30 min. After incubation, 5 μL buffer and 5 μL of KRas G12V-SA-Eu are added to each well of the ARP. After a 30-minute equilibration phase at room temperature, 5 μL of GST-cRAF-anti-GST-APC is added to each well. The final concentrations in the assay are 10 nM KRas G12V, 10 nM cRAF. The reaction is allowed to proceed for 60-90 minutes after which the plate is read on a PHERAstar FSX plate reader (exc: 337 nm, em 1: 665 nm, em 2: 620 nm). After normalization of the raw data (ratio of Signal₆₆₅ₙₘ/Signal₆₂₀ₙₘ) the data is fit to a four-parameter logistic curve in GraphPad Prism (v9.4.1).

### EXEMPLARY EMBODIMENTS

### P01 Embodiments

Embodiment 1. A compound of Formula (I):

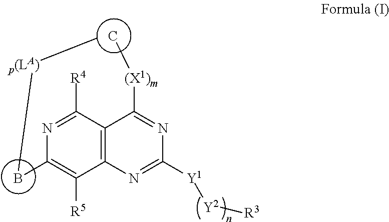

or a pharmaceutically acceptable salt thereof, wherein:
m is 0, 1, or 2;
each X¹ is independently selected from the group consisting of CH₂, CHR^{a}, C(R^{a})₂, O, N(R^{d}), C(═O), and S(O)₀₋₂, provided that —(X¹)ₘ— does not include any O—O, O—N, N—S(O)₀, or O—S(O)₀₋₂ bonds;
Ring C is selected from the group consisting of: C₃₋₁₅ cycloalkylene, 4-15 membered heterocyclylene, 6-15 membered arylene, and 5-15 membered heteroarylene, each of which is optionally substituted with 1-4 R⁷, wherein:
each R⁷ is independently selected from the group consisting of R^{a} and R^{b};
p is an integer from 3 to 10;
each L^{A} is independently selected from the group consisting of: L^{A1}, L^{A2}, L^{A3}, and L^{A4} provided that 0-2 occurrences of L^{A} are L^{A4}, and 0-3 occurrences of L^{A} are selected from the group consisting of L^{A2} and L^{A3};
each L^{A1} is independently selected from the group consisting of: —CH₂—, —CHR^{L}—, and —C(R^{L})₂—, wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, and C₁₋₆ alkyl optionally substituted with 1-6 R^{c};
each L^{A2} is independently selected from the group consisting of:

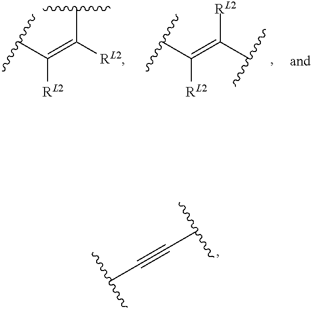

wherein each R^{L2} is independently selected from the group consisting of: H, halo, CN, and C₁₋₆ alkyl optionally substituted with 1-6 R^{c};
each L^{A3} is independently selected from the group consisting of: —N(R^{d})—, —O—, —S(O)₀₋₂—, and C(═O);
each L^{A4} is independently selected from the group consisting of: C₃₋₁₀ cycloalkylene, 4-10 membered heterocyclylene, C₆₋₁₀ arylene, and 5-10 membered heteroarylene, each of which is optionally substituted with 1-3 R^{g};
Ring B is selected from the group consisting of: C₆₋₁₀ arylene and 5-10 membered heteroarylene, each of which is optionally substituted with 1-6 substituents independently selected from the group consisting of: R^{a} and R^{b};
Y¹ is selected from the group consisting of: a bond, —N(H)—, —N(C₁₋₃ alkyl)-, —N(C₃₋₆ cycloalkyl)-, —O—, and —S(O)₀₋₂—;
n is 0 or 1;
Y² is a straight-chain C₁₋₆ alkylene optionally substituted with 1-6 R^{Y};
each R^{Y} is independently selected from the group consisting of: halo, cyano, —OH, oxo, C₁₋₆ alkoxy, C₁₋₆ haloalkoxy, C₁₋₆ alkyl, and C₁₋₆ haloalkyl, or
a pair of R^{Y} on the same or different carbon atom(s) taken together with the atom(s) connecting them form a C₃₋₆ cycloalkyl ring or 4-6 membered heterocyclyl ring, each of which is optionally substituted with 1-3 independently selected C₁₋₃ alkyl;
R³ is selected from the group consisting of:
(a) —H;
(b) -halo;
(c) —NR^{d}R^{e};
(d) C₃₋₁₀ cycloalkyl or 3-15 membered heterocyclyl, each of which is optionally substituted with 1-6 substituents independently selected from the group consisting of: R^{a} and R^{b}; and
(e) C₆₋₁₀ aryl or 5-10 membered heteroaryl, each of which is optionally substituted with 1-6 substituents independently selected from the group consisting of: R^{a} and R^{b};
R⁴ and R⁵ are independently selected from the group consisting of:
(a) —H;
(b) halo;
(c) cyano;
(d) C₁₋₆ alkyl;
(e) C₁₋₆ haloalkyl;
(f) C₁₋₆ alkoxy;
(g) C₁₋₆ haloalkoxy;
(h) —(C₀₋₃ alkylene)-C₃₋₆ cycloalkyl, wherein the C₃₋₆ cycloalkyl portion is optionally substituted with 1-3 R^{g}; and
(i) —O—(C₀₋₃ alkylene)-C₃₋₆ cycloalkyl, wherein the C₃₋₆ cycloalkyl portion is optionally substituted with 1-3 R^{g};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
b is 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, cyano, —OH, —C₁₋₆ alkoxy, —C₁₋₆ haloalkoxy, —NH₂, —N(H)(C₁₋₃ alkyl), and —N(C₁₋₃ alkyl)₂.

Embodiment 2. The compound of Embodiment 1, wherein m is 0.

Embodiment 3. The compound of Embodiments 1 or 2, wherein Ring C is a 4-15 membered heterocyclylene optionally substituted with 1-4 R⁷.

Embodiment 4. The compound of any one of Embodiments 1-3, wherein Ring C is a 4-6 membered heterocyclylene optionally substituted with 1-3 R⁷.

Embodiment 5. The compound of any one of Embodiments 1-4, wherein Ring C is a piperidinylene optionally substituted with 1-3 R⁷.

Embodiment 6. The compound of any one of Embodiments 1-5, wherein Ring C is:

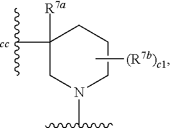

wherein:
R^{7a} is H or R⁷;
c1 is 0, 1, or 2;
each R^{7b} is independently R⁷; and
cc represents the point of attachment to -(L^{A})ₚ-.

Embodiment 7. The compound of Embodiment 6, wherein c1 is 0.

Embodiment 8. The compound of Embodiments 6 or 7, wherein R^{7a} is H or C₁₋₃ alkyl optionally substituted with 1-3 F.

Embodiment 9. The compound of any one of Embodiments 6-8, wherein R^{7a} is H.

Embodiment 10. The compound of Embodiments 6 or 7, wherein R^{7a} is —OH.

Embodiment 11. The compound of any one of Embodiments 1-10, wherein p is 4, 5, or 6.

Embodiment 12. The compound of any one of Embodiments 1-10, wherein p is 6, 7, or 8.

Embodiment 13. The compound of any one of Embodiments 1-12, wherein each L^{A} is independently selected from the group consisting of: L^{A1}, L^{A3}, and L^{A4}.

Embodiment 14. The compound of any one of Embodiments 1-13, wherein 0-1 occurrence of L^{A} is L^{A4}; and each remaining L^{A} is independently selected from the group consisting of L^{A1} and L^{A3}.

Embodiment 15. The compound of any one of Embodiments 1-14, wherein one occurrence of L^{A} is L^{A4}; and each remaining L^{A} is independently L^{A1}.

Embodiment 16. The compound of any one of Embodiments 1-14, wherein each L^{A} is independently L^{A1}.

Embodiment 17. The compound of any one of Embodiments 1-14, wherein 1-2 occurrence of L^{A} is independently L^{A3}; and each remaining L^{A} is independently L^{A1}.

Embodiment 18. The compound of any one of Embodiments 1-10, wherein p is 4, 5, or 6; one occurrence of L^{A} is L^{A4}; and each remaining L^{A} is independently L^{A1}.

Embodiment 19. The compound of any one of Embodiments 1-10 or 18, wherein -(L^{A})ₚ- is -(L^{A1})₀₋₅-L^{A4}-(L^{A1})₀₋₅, wherein p is 4, 5, or 6.

Embodiment 20. The compound of Embodiments 18 or 19, wherein L^{A4} is phenylene or 5-6 membered heteroarylene, each of which is optionally substituted with 1-3 R^{g}.

Embodiment 21. The compound of any one of Embodiments 18-20, wherein L^{A4} is 5-membered heteroarylene optionally substituted with 1-2 R^{g}.

Embodiment 22. The compound of any one of Embodiments 18-21, wherein L^{A4} is triazolylene or oxadiazolylene (e.g., 1,2,4-oxadiazolylene).

Embodiment 23. The compound of any one of Embodiments 19-22, wherein each L^{A1} is CH₂.

Embodiment 24. The compound of Embodiment 19, wherein -(L^{A})ₚ- is: -(L^{A1a})ₐ₁ₐ-L^{A4}-(L^{A1b})_{a1b-bb}, wherein:
a1a and a1b are independently integers from 0 to 5, provided that a1a+a1b is 3, 4, or 5;
L^{A1a} and L^{A1b} are independently L^{A1};
L^{A4} is phenylene or 5-6 membered heteroarylene, each of which is optionally substituted with 1-3 R^{g}; and
bb represents the point of attachment to Ring C.

Embodiment 25. The compound of Embodiment 24, wherein L^{A4} is 5-membered heteroarylene optionally substituted with 1-2 R^{g}.

Embodiment 26. The compound of Embodiments 24 or 25, wherein L^{A4} is selected from the group consisting of

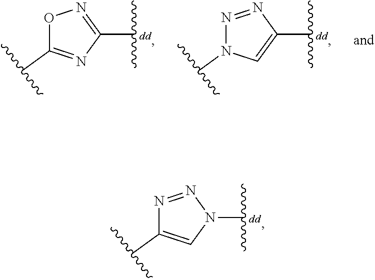

wherein dd represents the point of attachment to -(L^{A1b})_{a1b}-.

Embodiment 27. The compound of any one of Embodiments 24-26, wherein L^{A4} is

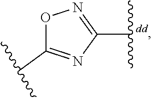

wherein dd represents the point of attachment to -(L^{A1b})_{a1b}-.

Embodiment 28. The compound of any one of Embodiments 24-27, wherein a1b is 1; and a1a is 2, 3, or 4.

Embodiment 29. The compound of any one of Embodiments 24-28, wherein each of L^{A1a} and L^{A1b} is CH₂.

Embodiment 30. The compound of any one of Embodiments 1-10, wherein p is 6, 7, or 8; and each L^{A} is independently L^{A1}.

Embodiment 31. The compound of Embodiment 30, wherein each L^{A} is —CH₂—.

Embodiment 32. The compound of any one of Embodiments 1-10, wherein p is 6, 7, or 8; one occurrence of L^{A} is C(═O); a second occurrence of L^{A} is N(R^{d}); and each remaining L^{A} is independently L^{A1}.

Embodiment 33. The compound of Embodiment 32, wherein -(L^{A})ₚ- is: -(L^{A1a})ₐ₁ₐ-C(═O)NH-(L^{A1b})_{a1b-bb}, wherein:
a1a and a1b are independently integers from 0 to 5, provided that a1a+a1b is 4, 5, or 6;
L^{A1a} and L^{A1b} are independently L^{A1}; and
bb represents the point of attachment to Ring C.

Embodiment 34. The compound of Embodiment 33, wherein a1b is 2; and a1a is 2, 3, or 4.

Embodiment 35. The compound of Embodiments 33 or 34, wherein each of L^{A1a} and L^{A1b} is CH₂.

Embodiment 36. The compound of Embodiment 1, wherein:
m is 0;
Ring C is:

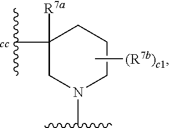

wherein:
R^{7a} is H or R⁷;
c1 is 0, 1, or 2;
each R^{7b} is independently R⁷; and
cc represents the point of attachment to -(L^{A})ₚ-;
-(L^{A})ₚ- is: -(L^{A1a})ₐ₁ₐ-L^{A4}-(L^{A1b})_{a1b-bb}, wherein:
a1a and a1b are independently integers from 0 to 5, provided that a1a+a1b is 3, 4, or 5;
L^{A1a} and L^{A1b} are independently L^{A1};
L^{A4} is phenylene or 5-6 membered heteroarylene, each of which is optionally substituted with 1-3 R^{g}; and
bb represents the point of attachment to Ring C.

Embodiment 37. The compound of Embodiment 36, wherein L^{A4} is 5-membered heteroarylene optionally substituted with 1-2 R^{g}.

Embodiment 38. The compound of Embodiments 36 or 37, wherein L^{A4} is

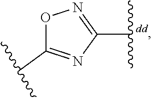

wherein dd represents the point of attachment to -(L^{A1b})_{a1b}-.

Embodiment 39. The compound of any one of Embodiments 36-38, wherein a1b is 1; and a1a is 2, 3, or 4.

Embodiment 40. The compound of any one of Embodiments 36-39, wherein each of L^{A1a} and L^{A1b} is CH₂.

Embodiment 41. The compound of Embodiment 1, wherein:
m is 0;
Ring C is:

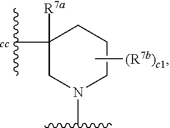

wherein:
R^{7a} is H or R⁷;
c1 is 0, 1, or 2;
each R^{7b} is independently R⁷; and
cc represents the point of attachment to -(L^{A})ₚ-;
p is 6, 7, or 8; and
each L^{A} is independently L^{A1}.

Embodiment 42. The compound of Embodiment 41, wherein each L^{A} is —CH₂—.

Embodiment 43. The compound of Embodiment 1, wherein:
m is 0;
Ring C is:

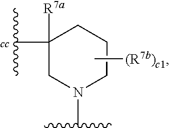

wherein:
R^{7a} is H or R⁷;
c1 is 0, 1, or 2;
each R^{7b} is independently R⁷; and
cc represents the point of attachment to -(L^{A})ₚ-;
-(L^{A})ₚ- is: -(L^{A1a})ₐ₁ₐ-C(═O)NH-(L^{A1b})_{a1b-bb}, wherein:
a1a and a1b are independently integers from 0 to 5, provided that a1a+a1b is 4, 5, or 6;
L^{A1a} and L^{A1b} are independently L^{A1}; and
bb represents the point of attachment to Ring C.

Embodiment 44. The compound of Embodiment 43, wherein a1b is 2; and a1a is 2, 3, or 4.

Embodiment 45. The compound of Embodiments 43 or 44, wherein each of L^{A1a} and L^{A1b} is CH₂.

Embodiment 46. The compound of any one of Embodiments 36-46, wherein R^{7a} is H.

Embodiment 47. The compound of any one of Embodiments 36-46, wherein R^{7a} is —OH.

Embodiment 48. The compound of any one of Embodiments 36-47, wherein c1 is 0.

Embodiment 49. The compound of any one of Embodiments 1-48, wherein Ring B is C₆₋₁₀ arylene optionally substituted with 1-6 substituents independently selected from the group consisting of: R^{a} and R^{b}.

Embodiment 50. The compound of any one of Embodiments 1-49, wherein Ring B is naphthylene optionally substituted with 1-4 R^{a}.

Embodiment 51. The compound of any one of Embodiments 1-50, wherein Ring B is

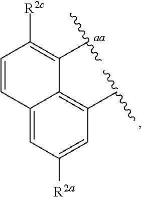

wherein R^{2a} and R^{2c} are independently H or R^{a}; and aa represents the point of attachment to -(L^{A})ₚ-.

Embodiment 52. The compound of Embodiment 51, wherein R^{2a} is —OH.

Embodiment 53. The compound of Embodiments 51 or 52, wherein R^{2c} is halo (e.g., F) or H.

Embodiment 54. The compound of any one of Embodiments 1-53, wherein Ring B is

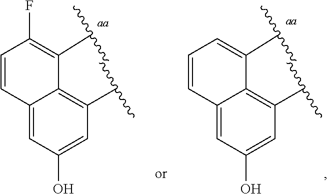

wherein aa represents the point of attachment to -(L^{A})ₚ-.

Embodiment 55. The compound of any one of Embodiments 1-49, wherein Ring B is phenylene optionally substituted with 1-4 R^{a}.

Embodiment 56. The compound of any one of Embodiments 1-49 or 55, wherein Ring B is

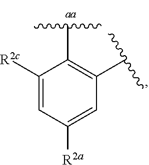

wherein R^{2a} and R^{2c} are independently H or R^{a}; and aa represents the point of attachment to -(L^{A})ₚ-.

Embodiment 57. The compound of Embodiment 56, wherein R^{2a} is —OH.

Embodiment 58. The compound of Embodiments 56 or 57, wherein R^{2c} is halo (e.g., —Cl).

Embodiment 59. The compound of any one of Embodiments 1-58, wherein Y is —O—.

Embodiment 60. The compound of any one of Embodiments 1-59, wherein n is 1.

Embodiment 61. The compound of any one of Embodiments 1-60, wherein Y² is a straight-chain C₁₋₃ alkylene optionally substituted with 1-3 R^{Y}.

Embodiment 62. The compound of any one of Embodiments 1-61, wherein Y² is —CH₂—.

Embodiment 63. The compound of any one of Embodiments 1-61, wherein Y² is

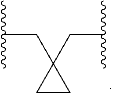 [SMILES: *CC1(C*)CC1]

Embodiment 64. The compound of any one of Embodiments 1-63, wherein R³ is a 4-10 membered heterocyclyl optionally substituted with 1-6 substituents independently selected from the group consisting of: R^{a} and R^{b}.

Embodiment 65. The compound of any one of Embodiments 1-64, wherein R³ is a monocyclic 4-6 membered heterocyclyl optionally substituted with 1-3 R^{a}, wherein the heterocyclyl includes at least one ring nitrogen atom.

Embodiment 66. The compound of any one of Embodiments 1-65, wherein R³ is

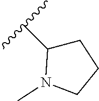 [SMILES: *C1CCCN1C]

optionally substituted with 1-3 R^{a} on one or more ring carbon atoms.

Embodiment 67. The compound of any one of Embodiments 1-65, wherein R³ is

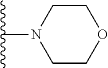 [SMILES: *N1CCOCC1]

optionally substituted with 1-3 R^{a} on one or more ring carbon atoms.

Embodiment 68. The compound of any one of Embodiments 1-64, wherein R³ is a bicyclic 7-10 membered heterocyclyl optionally substituted with 1-6 R^{a}.

Embodiment 69. The compound of any one of Embodiments 1-64 or 68, wherein R³ is

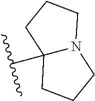 [SMILES: *C12CCCN1CCC2]

optionally substituted with 1-3 R^{a}.

Embodiment 70. The compound of any one of Embodiments 1-64 or 68-69, wherein R³ is selected from the group consisting of:

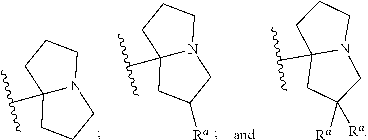

Embodiment 71. The compound of any one of Embodiments 1-64 or 68-70, wherein R³ is

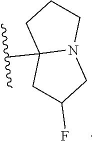 [SMILES: *C12CCCN1CC(F)C2]

Embodiment 72. The compound of any one of Embodiments 1-64 or 68, wherein R³ is selected from the group consisting of:

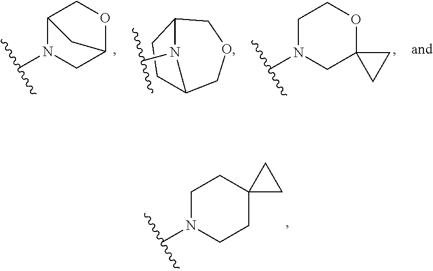

each of which is optionally substituted with 1-3 R^{a}.

Embodiment 73. The compound of any one of Embodiments 1-58, wherein Y is —O—; n is 1; Y² is —CH₂—; and R³ is

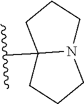 [SMILES: *C12CCCN1CCC2]

optionally substituted with 1-3 R^{a}.

Embodiment 74. The compound of Embodiment 73, wherein R³ is selected from the group consisting of:

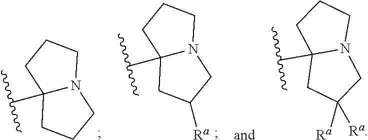

Embodiment 75. The compound of Embodiments 73 or 74, wherein R³ is

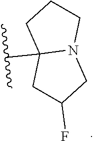 [SMILES: *C12CCCN1CC(F)C2]

Embodiment 76. The compound of any one of Embodiments 1-58, wherein Y¹ is —O—; n is 1; Y² is

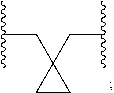 [SMILES: *CC1(C*)CC1]

and R³ is a 5-8 membered heterocyclyl optionally substituted with 1-3 R^{a}.

Embodiment 77. The compound of Embodiment 76, wherein R³ is selected from the group consisting of:

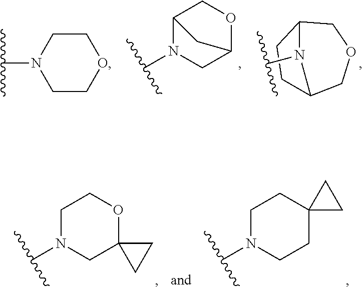

each of which is optionally substituted with 1-3 R^{a}.

Embodiment 78. The compound of any one of Embodiments 1-77, wherein R⁴ is H.

Embodiment 79. The compound of any one of Embodiments 1-78, wherein R⁵ is —F.

Embodiment 80. The compound of any one of Embodiments 1-77, wherein R⁴ is H; and R⁵ is —F.

Embodiment 81. The compound of Embodiment 1, wherein the compound is a compound of Formula (I-a):

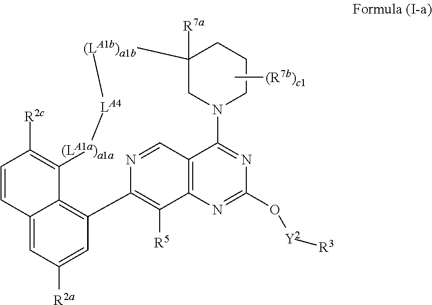

or a pharmaceutically acceptable salt thereof, wherein:
R^{7a} is H or R⁷;
c1 is 0, 1, or 2;
each R^{7b} is independently R⁷;
a1a and a1b are independently integers from 0 to 5, provided that a1a+a1b is 3, 4, or 5;
L^{A1a} and L^{A1b} are independently L^{A1};
L^{A4} is phenylene or 5-6 membered heteroarylene, each of which is optionally substituted with 1-3 R^{g};
R^{2a} and R^{2c} are independently H or R^{a};

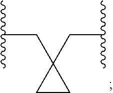 [SMILES: *CC1(C*)CC1]

Y² is —CH₂— or
R³ is a 5-8 membered heterocyclyl optionally substituted with 1-3 R^{a}; and
R⁵ is H or halo.

Embodiment 82. The compound of Embodiment 81, wherein L^{A4} is 5-membered heteroarylene optionally substituted with 1-2 R^{g}.

Embodiment 83. The compound of Embodiments 81 or 82, wherein L^{A4} is

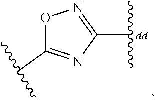

wherein dd represents the point of attachment to -(L^{A1b})_{a1b}-.

Embodiment 84. The compound of any one of Embodiments 81-83, wherein a1b is 1; and a1a is 2, 3, or 4.

Embodiment 85. The compound of any one of Embodiments 81-84, wherein each of L^{A1a} and L^{A1b} is CH₂.

Embodiment 86. The compound of Embodiment 1, wherein the compound is a compound of Formula (I-b):

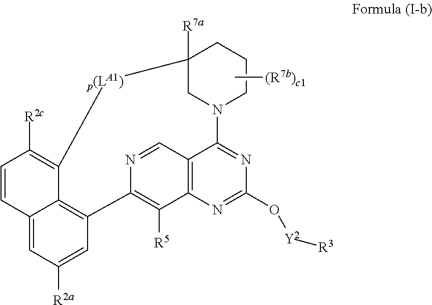

or a pharmaceutically acceptable salt thereof, wherein:
R^{7a} is H or R⁷;
c1 is 0, 1, or 2;
each R^{7b} is independently R⁷;
p is 6, 7, or 8;
R^{2a} and R^{2c} are independently H or R^{a};
Y² is —CH₂— or

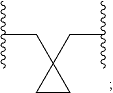 [SMILES: *CC1(C*)CC1]

R³ is a 5-8 membered heterocyclyl optionally substituted with 1-3 R^{a}; and
R⁵ is H or halo.

Embodiment 87. The compound of Embodiment 86, wherein each L^{A1} is CH₂.

Embodiment 88. The compound of Embodiment 1, wherein the compound is a compound of Formula (I-c):

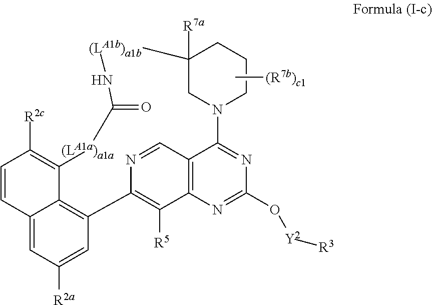

or a pharmaceutically acceptable salt thereof, wherein:
R^{7a} is H or R⁷;
c1 is 0, 1, or 2;
each R^{7b} is independently R⁷;
a1a and a1b are independently integers from 0 to 5, provided that a1a+a1b is 4, 5, or
6;
L^{A1a} and L^{A1b} are independently L^{A1};
R^{2a} and R^{2c} are independently H or R^{a};
Y² is —CH₂— or

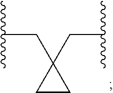 [SMILES: *CC1(C*)CC1]

R³ is a 5-8 membered heterocyclyl optionally substituted with 1-3 R^{a}; and
R⁵ is H or halo.

Embodiment 89. The compound of Embodiment 88, wherein a1b is 2; and a1a is 2, 3, or 4.

Embodiment 90. The compound of Embodiments 88 or 89, wherein each of L^{A1a} and L^{A1b} is CH₂.

Embodiment 91. The compound of any one of Embodiments 81-90, wherein: R^{7a} is H or OH; c1 is 0; R^{2a} is —OH; and R^{2b} is H or —F.

Embodiment 92. The compound of any one of Embodiments 81-91, wherein Y² is CH₂; R³ is

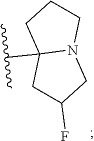 [SMILES: *C12CCCN1CC(F)C2]

### and R⁵ is F.

Embodiment 93. The compound of Embodiment 1, wherein the compound is selected from the group consisting of Compound No. 101, 101a, 102, 102a, 103, 103a, 104, 104a, 105, 105a, 106, 106a, 107, 107a, 108, 108a, 109, 109a, 110, 110a, 111, 111a, 112, 112a, 113, 113a, 114, 114a, 115, 115a, 116, 116a, 117, 117a, 118, 118a, 119, 119a, 120, 120a, 121, 121a, 122, 122a, 123, 123a, 124, 124a, 125, 125a, 126, 126a, 127, 127a, 128, 128a, 129, 129a, 130, 130a, 131, 131a, 132, 132a, 133, 133a, 134, and 134a, as depicted in Table C1, or a pharmaceutically acceptable salt thereof.

Embodiment 94. A pharmaceutical composition comprising a compound of any one of Embodiments 1-93, or a pharmaceutically acceptable salt thereof, and a pharmaceutically acceptable excipient.

Embodiment 95. A dysregulated KRas protein non-covalently bound with a compound of any one of Embodiments 1-93, or a pharmaceutically acceptable salt thereof.

Embodiment 96. A method for treating a KRas-associated cancer in a subject in need thereof, the method comprising administering to the subject a therapeutically effective amount of a compound of any one of Embodiments 1-93, or a pharmaceutically acceptable salt thereof, or a pharmaceutical composition according to Embodiment 94.

Embodiment 97. A method for treating a KRas-associated cancer in a subject in need thereof, the method comprising (a) determining that the cancer is a KRas-associated cancer; and (b) administering to the subject a therapeutically effective amount of a compound of any one of Embodiments 1-93, or a pharmaceutically acceptable salt thereof, or a pharmaceutical composition according to Embodiment 94.

Embodiment 98. A method of treating a KRas-associated cancer in a subject, the method comprising administering to a subject identified or diagnosed as having a mutant KRas-associated cancer a therapeutically effective amount of a compound of any one of Embodiments 1-93 or a pharmaceutically acceptable salt thereof, or a pharmaceutical composition according to Embodiment 94.

Embodiment 99. A method of treating a KRas-associated cancer in a subject, the method comprising:
(a) determining that the cancer in the subject is a KRas-associated cancer; and
(b) administering to the subject a therapeutically effective amount of a compound of any one of Embodiments 1-93 or a pharmaceutically acceptable salt thereof, or a pharmaceutical composition according to Embodiment 94.

Embodiment 100. The method of any one of Embodiments 96-99, wherein the KRas-associated cancer is a mutant KRas-associated cancer.

Embodiment 101. The method of any one of Embodiments 97 or 99, wherein the step of determining that the cancer in the subject is a KRas-associated cancer includes performing an assay to detect a KRas dysregulation (e.g., a KRas mutation or amplification) in a sample from the subject.

Embodiment 102. The method of Embodiment 101, wherein the KRas-associated cancer is a mutant KRas-associated cancer.

### P04 Embodiments

Embodiment 1. A compound of Formula (A):

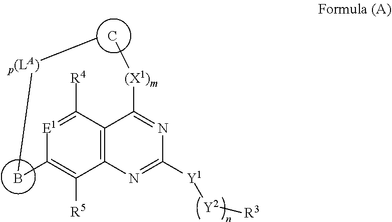

or a pharmaceutically acceptable salt thereof, wherein:
m is 0, 1, or 2;
each X¹ is independently selected from the group consisting of CH₂, CHR^{a}, C(R^{a})₂, O, N(R^{d}), C(═O), and S(O)₀₋₂, provided that —(X¹)ₘ— does not include any O—O, O—N, N—S(O)₀, or O—S(O)₀₋₂ bonds;
Ring C is selected from the group consisting of: C₃₋₁₅ cycloalkylene, 4-15 membered heterocyclylene, 6-15 membered arylene, and 5-15 membered heteroarylene, each of which is optionally substituted with 1-4 R⁷, wherein:
each R⁷ is independently selected from the group consisting of R^{a} and R^{b};
p is an integer from 3 to 10;
each L^{A} is independently selected from the group consisting of: L^{A1}, L^{A2}, L^{A3}, and L^{A4}, provided that 0-2 occurrences of L^{A} are L^{A4}, and 0-3 occurrences of L^{A} are selected from the group consisting of L^{A2} and L^{A3};
each L^{A1} is independently selected from the group consisting of: —CH₂—, —CHR^{L}—, and —C(R^{L})₂—, wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, and C₁₋₆ alkyl optionally substituted with 1-6 R^{c};
each L^{A2} is independently selected from the group consisting of:

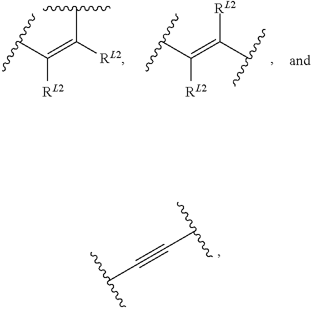

wherein each R^{L2} is independently selected from the group consisting of: H, halo, CN, and C₁₋₆ alkyl optionally substituted with 1-6 R^{c};
each L^{A3} is independently selected from the group consisting of: —N(R^{d})—, —O—, —S(O)₀₋₂—, and C(═O);
each L^{A4} is independently selected from the group consisting of: C₃₋₁₀ cycloalkylene, 4-10 membered heterocyclylene, C₆₋₁₀ arylene, and 5-10 membered heteroarylene, each of which is optionally substituted with 1-3 R^{g};
Ring B is selected from the group consisting of: C₆₋₁₀ arylene and 5-10 membered heteroarylene, each of which is optionally substituted with 1-6 substituents independently selected from the group consisting of: R^{a} and R^{b};
Y¹ is selected from the group consisting of: a bond, —N(H)—, —N(C₁₋₃ alkyl)-, —N(C₃₋₆ cycloalkyl)-, —O—, and —S(O)₀₋₂—;
n is 0 or 1;
Y² is a straight-chain C₁₋₆ alkylene optionally substituted with 1-6 R^{Y}; each R^{Y} is independently selected from the group consisting of: halo, cyano, —OH, oxo, C₁₋₆ alkoxy, C₁₋₆ haloalkoxy, C₁₋₆ alkyl, and C₁₋₆ haloalkyl, or
a pair of R^{Y} on the same or different carbon atom(s) taken together with the atom(s) connecting them form a C₃₋₆ cycloalkyl ring or 4-6 membered heterocyclyl ring, each of which is optionally substituted with 1-3 independently selected C₁₋₃ alkyl;
R³ is selected from the group consisting of:
(a) —H;
(b) -halo;
(c) —NR^{d}R^{e};
(d) C₃₋₁₀ cycloalkyl or 3-15 membered heterocyclyl, each of which is optionally substituted with 1-6 substituents independently selected from the group consisting of: R^{a} and R^{b}; and
(e) C₆₋₁₀ aryl or 5-10 membered heteroaryl, each of which is optionally substituted with 1-6 substituents independently selected from the group consisting of: R^{a} and R^{b};
E¹ is N, CH, or CR⁶;
R⁴, R⁵, and R⁶ are independently selected from the group consisting of:
(a) —H;
(b) halo;
(c) cyano;
(d) C₁₋₆ alkyl;
(e) C₁₋₆ haloalkyl;
(f) C₁₋₆ alkoxy;
(g) C₁₋₆ haloalkoxy;
(h) —(C₀₋₃ alkylene)-C₃₋₆ cycloalkyl, wherein the C₃₋₆ cycloalkyl portion is optionally substituted with 1-3 R^{g}; and
(i) —O—(C₀₋₃ alkylene)-C₃₋₆ cycloalkyl, wherein the C₃₋₆ cycloalkyl portion is optionally substituted with 1-3 R^{g};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
b is 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, cyano, —OH, —C₁₋₆ alkoxy, —C₁₋₆ haloalkoxy, —NH₂, —N(H)(C₁₋₃ alkyl), and —N(C₁₋₃ alkyl)₂.

Embodiment 2. The compound of Embodiment 1, wherein the compound of Formula (A) is a compound of Formula (I):

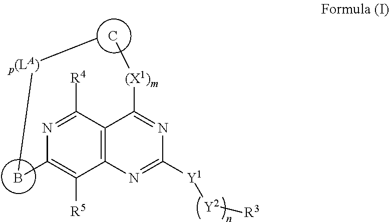

or a pharmaceutically acceptable salt thereof.

Embodiment 3. The compound of Embodiment 1, wherein the compound of Formula (A) is a compound of Formula (II):

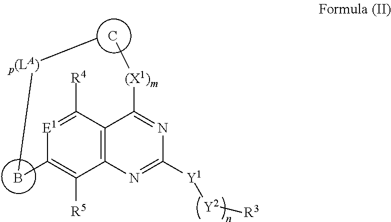

or a pharmaceutically acceptable salt thereof, wherein E¹ is CH or CR⁶.

Embodiment 4. The compound of any one of Embodiments 1-3, wherein m is 0.

Embodiment 5. The compound of any one of Embodiments 1-4, wherein Ring C is a 4-15 membered heterocyclylene optionally substituted with 1-4 R⁷.

Embodiment 6. The compound of any one of Embodiments 1-5, wherein Ring C is a 4-6 membered heterocyclylene optionally substituted with 1-3 R⁷.

Embodiment 7. The compound of any one of Embodiments 1-6, wherein Ring C is a piperidinylene optionally substituted with 1-3 R⁷.

Embodiment 8. The compound of any one of Embodiments 1-7, wherein Ring C is:

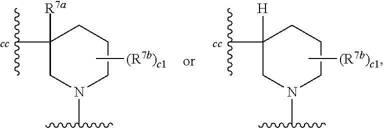

wherein:
c1 is 0, 1, or 2;
each R^{7a} and R^{7b} is an independently selected R⁷; and
cc represents the point of attachment to -(L^{A})ₚ-.

Embodiment 9. The compound of Embodiment 8, wherein c1 is 0.

Embodiment 10. The compound of Embodiments 8 or 9, wherein Ring C is

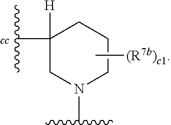

Embodiment 11. The compound of Embodiments 8 or 9, wherein Ring C is

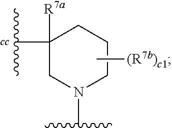

and R^{7a} is C₁₋₃ alkyl optionally substituted with 1-3 F.

Embodiment 12. The compound of Embodiments 8 or 9, wherein Ring C is

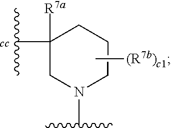

### and R^{7a} is —OH.

Embodiment 13. The compound of any one of Embodiments 1-5, wherein Ring C is a 7-10 membered heterocyclylene optionally substituted with 1-3 R⁷.

Embodiment 14. The compound of Embodiment 13, wherein Ring C has one ring nitrogen atom, one ring oxygen atom, and no additional ring heteroatoms.

Embodiment 15. The compound of any one of Embodiments 1-5 or 13-14, wherein Ring C is

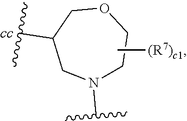

wherein c1 is 0, 1, or 2; and cc represents the point of attachment to -(L^{A})ₚ-.

Embodiment 16. The compound of any one of Embodiments 1-15, wherein p is 4, 5, or 6.

Embodiment 17. The compound of any one of Embodiments 1-15, wherein p is 6, 7, or 8.

Embodiment 18. The compound of any one of Embodiments 1-17, wherein each L^{A} is independently selected from the group consisting of: L^{A1}, L^{A3}, and L^{A4}.

Embodiment 19. The compound of any one of Embodiments 1-18, wherein 0-1 occurrence of L^{A} is L^{A4}; and each remaining L^{A} is independently selected from the group consisting of L^{A1} and L^{A3}.

Embodiment 20. The compound of any one of Embodiments 1-19, wherein one occurrence of L^{A} is L^{A4}; and each remaining L^{A} is independently L^{A1}.

Embodiment 21. The compound of any one of Embodiments 1-19, wherein each L^{A} is independently L^{A1}.

Embodiment 22. The compound of any one of Embodiments 1-19, wherein 1-2 occurrence of L^{A} is independently L^{A3}; and each remaining L^{A} is independently L^{A1}.

Embodiment 23. The compound of any one of Embodiments 1-15, wherein p is 4, 5, or 6; one occurrence of L^{A} is L^{A4}; and each remaining L^{A} is independently L^{A1}.

Embodiment 24. The compound of any one of Embodiments 1-15, wherein p is 6, 7, or 8; and each L^{A} is independently L^{A1}.

Embodiment 25. The compound of Embodiment 24, wherein each L^{A} is —CH₂—.

Embodiment 26. The compound of any one of Embodiments 1-15, wherein p is 6, 7, or 8; one occurrence of L^{A} is C(═O); a second occurrence of L^{A} is N(R^{d}) (e.g., NH); and each remaining L^{A} is independently L^{A1}.

Embodiment 27. The compound of any one of Embodiments 1-15 or 26, wherein -(L^{A})ₚ- is: -(L^{A1a})ₐ₁ₐ-C(═O)NH-(L^{A1b})_{a1b-bb}, wherein:
a1a and a1b are independently integers from 0 to 5, provided that a1a+a1b is 4, 5, or 6;
each L^{A1a} and L^{A1b} is an independently selected L^{A1}; and
bb represents the point of attachment to Ring C.

Embodiment 28. The compound of Embodiment 27, wherein a1b is 2; and a1a is 2, 3, or 4.

Embodiment 29. The compound of Embodiment 27, wherein a1b is 1; and a1a is 3, 4, or 5.

Embodiment 30. The compound of any one of Embodiments 27-29, wherein each of L^{A1a} and L^{A1b} is CH₂.

Embodiment 31. The compound of any one of Embodiments 1-3, wherein:
m is 0;
Ring C is:

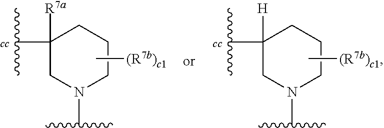

wherein:
c1 is 0, 1, or 2;
each R^{7a} and R^{7b} is an independently selected R⁷; and
cc represents the point of attachment to -(L^{A})ₚ-;
-(L^{A})ₚ- is: -(L^{A1a})ₐ₁ₐ-C(═O)NH-(L^{A1b})_{a1b-bb}, wherein:
a1a and a1b are independently integers from 0 to 5, provided that a1a+a1b is 4, 5, or 6;
each L^{A1a} and L^{A1b} is independently selected L^{A1}; and
bb represents the point of attachment to Ring C.

Embodiment 32. The compound of Embodiment 31, wherein a1b is 2; and a1a is 2, 3, or 4.

Embodiment 33. The compound of Embodiment 31, wherein a1b is 1; and a1a is 3, 4, or 5.

Embodiment 34. The compound of any one of Embodiments 31-33, wherein each of L^{A1a} and L^{A1b} is CH₂.

Embodiment 35. The compound of any one of Embodiments 31-34, wherein Ring C is

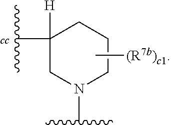

Embodiment 36. The compound of any one of Embodiments 31-34, wherein Ring C is

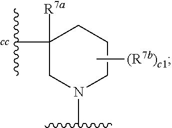

### and R^{7a} is —OH.

Embodiment 37. The compound of any one of Embodiments 31-36, wherein c1 is 0.

Embodiment 38. The compound of any one of Embodiments 1-3, wherein:
m is 0;
Ring C is:

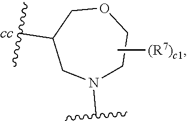

wherein:
c1 is 0, 1, or 2;
cc represents the point of attachment to -(L^{A})ₚ-; and
-(L^{A})ₚ- is: -(L^{A1a})ₐ₁ₐ-C(═O)NH-(L^{A1b})_{a1b-bb}, wherein:
a1a and a1b are independently integers from 0 to 5, provided that a1a+a1b is 4, 5, or 6;
each L^{A1a} and L^{A1b} is independently selected L^{A1}; and
bb represents the point of attachment to Ring C.

Embodiment 39. The compound of Embodiment 38, wherein a1b is 2; and a1a is 2, 3, or 4.

Embodiment 40. The compound of Embodiment 38, wherein a1b is 1; and a1a is 3, 4, or 5.

Embodiment 41. The compound of any one of Embodiments 38-40, wherein each of L^{A1a} and L^{A1b} is CH₂; and c1 is 0.

Embodiment 42. The compound of any one of Embodiments 1-41, wherein Ring B is a C₆₋₁₀ arylene optionally substituted with 1-6 substituents independently selected from the group consisting of: R^{a} and R^{b}.

Embodiment 43. The compound of any one of Embodiments 1-42, wherein Ring B is naphthylene optionally substituted with 1-4 R^{a}.

Embodiment 44. The compound of any one of Embodiments 1-43, wherein Ring B is

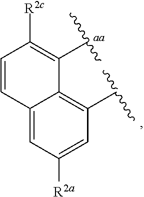

wherein R^{2a} and R^{2c} are independently H or R^{a}; and aa represents the point of attachment to -(L^{A})ₚ-.

Embodiment 45. The compound of Embodiment 44, wherein R^{2a} is —OH.

Embodiment 46. The compound of Embodiment 44, wherein R^{2a} is H or —NH₂.

Embodiment 47. The compound of any one of Embodiments 44-46, wherein R^{2c} is halo or H.

Embodiment 48. The compound of any one of Embodiments 1-44, wherein Ring B is

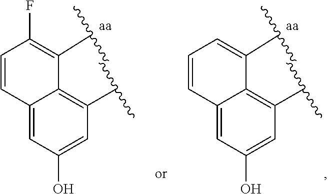

wherein aa represents the point of attachment to -(L^{A})ₚ-.

Embodiment 49. The compound of any one of Embodiments 1-44, wherein Ring B is

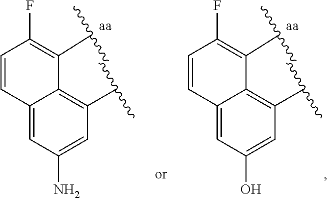

wherein aa represents the point of attachment to -(L^{A})ₚ-.

Embodiment 50. The compound of any one of Embodiments 1-42, wherein Ring B is phenylene optionally substituted with 1-4 R^{a}.

Embodiment 51. The compound of any one of Embodiments 1-42 or 50, wherein Ring B is

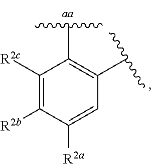

wherein R^{2a}, R^{2b}, and R^{2c} are independently H or R^{a}; and aa represents the point of attachment to -(L^{A})ₚ-.

Embodiment 52. The compound of any one of Embodiments 1-42 or 50-51, wherein Ring B is

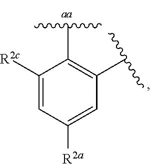

wherein R^{2a} and R^{2c} are independently H or R^{a}; and aa represents the point of attachment to -(L^{A})ₚ-.

Embodiment 53. The compound of Embodiments 51 or 52, wherein R^{2a} is —OH.

Embodiment 54. The compound of Embodiments 51 or 52, wherein R^{2a} is —NH₂.

Embodiment 55. The compound of any one of Embodiments 51-54, wherein R^{2c} is halo (e.g., —Cl).

Embodiment 56. The compound of any one of Embodiments 1-41, wherein Ring B is a 5-10 membered heteroarylene, which is optionally substituted with 1-6 substituents independently selected from the group consisting of: R^{a} and R^{b}.

Embodiment 57. The compound of any one of Embodiments 1-41 or 56, wherein Ring B is a 9-10 membered bicyclic heteroarylene, which is optionally substituted with 1-3 R^{a}.

Embodiment 58. The compound of any one of Embodiments 1-41 or 56-57, wherein Ring B is

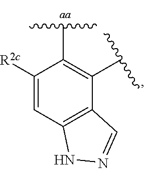

wherein R^{2c} is H or R^{a}; and aa represents the point of attachment to -(L^{A})ₚ-.

Embodiment 59. The compound of Embodiment 58, wherein R^{2c} is halo.

Embodiment 60. The compound of any one of Embodiments 1-41 or 56-59, wherein Ring B is

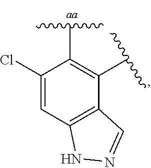

wherein aa represents the point of attachment to -(L^{A})ₚ-.

Embodiment 61. The compound of any one of Embodiments 1-60, wherein Y¹ is —O—.

Embodiment 62. The compound of any one of Embodiments 1-61, wherein n is 1.

Embodiment 63. The compound of any one of Embodiments 1-62, wherein Y² is a straight-chain C₁₋₃ alkylene optionally substituted with 1-3 R^{Y}.

Embodiment 64. The compound of any one of Embodiments 1-63, wherein Y² is —CH₂—.

Embodiment 65. The compound of any one of Embodiments 1-63, wherein Y² is

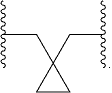

Embodiment 66. The compound of any one of Embodiments 1-65, wherein R³ is a 4-10 membered heterocyclyl optionally substituted with 1-6 substituents independently selected from the group consisting of: R^{a} and R^{b}.

Embodiment 67. The compound of any one of Embodiments 1-66, wherein R³ is a monocyclic 4-6 membered heterocyclyl optionally substituted with 1-3 R^{a}, wherein the heterocyclyl includes at least one ring nitrogen atom.

Embodiment 68. The compound of any one of Embodiments 1-67, wherein R³ is

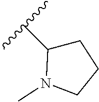 [SMILES: *C1CCCN1C]

optionally substituted with 1-3 R^{a} on one or more ring carbon atoms.

Embodiment 69. The compound of any one of Embodiments 1-67, wherein R³ is

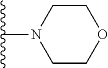 [SMILES: *N1CCOCC1]

optionally substituted with 1-3 R^{a}.

Embodiment 70. The compound of any one of Embodiments 1-66, wherein R³ is a bicyclic 7-10 membered heterocyclyl optionally substituted with 1-6 R^{a}.

Embodiment 71. The compound of any one of Embodiments 1-66 or 70, wherein R³ is

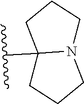 [SMILES: *C12CCCN1CCC2]

optionally substituted with 1-3 R^{a}.

Embodiment 72. The compound of any one of Embodiments 1-66 or 70-71, wherein R³ is selected from the group consisting of:

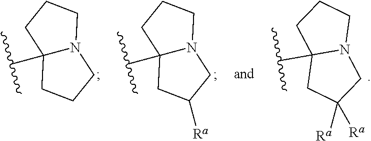

Embodiment 73. The compound of any one of Embodiments 1-66 or 70-72, wherein R³ is

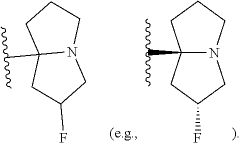

Embodiment 74. The compound of any one of Embodiments 1-66 or 70, wherein R³ is selected from the group consisting of:

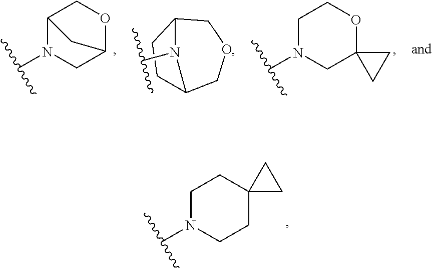

each of which is optionally substituted with 1-3 R^{a}.

Embodiment 75. The compound of any one of Embodiments 1-60, wherein Y is —O—; n is 1; Y² is —CH₂—; and R³ is

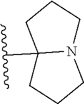 [SMILES: *C12CCCN1CCC2]

optionally substituted with 1-3 R^{a}.

Embodiment 76. The compound of Embodiment 75, wherein R³ is selected from the group consisting of:

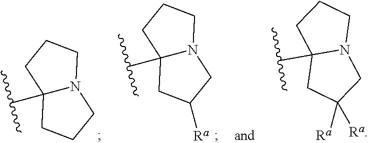

Embodiment 77. The compound of Embodiments 75 or 76, wherein R³ is

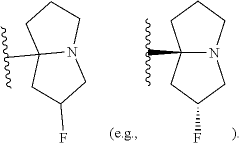

Embodiment 78. The compound of any one of Embodiments 1-60, wherein Y is —O—; n is 1; Y² is

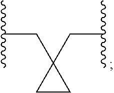

and R³ is a 5-8 membered heterocyclyl optionally substituted with 1-3 R^{a}.

Embodiment 79. The compound of Embodiment 78, wherein R³ is selected from the group consisting of:

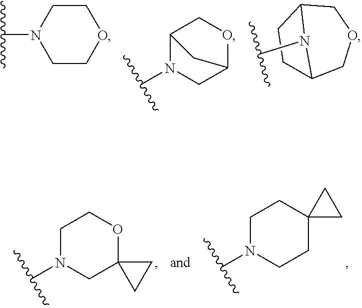

each of which is optionally substituted with 1-3 R^{a}.

Embodiment 80. The compound of any one of Embodiments 1-79, wherein R⁴ is H.

Embodiment 81. The compound of any one of Embodiments 1-80, wherein R⁵ is —F.

Embodiment 82. The compound of any one of Embodiments 1-79, wherein R⁴ is H; and R⁵ is —F.

Embodiment 83. The compound of any one of Embodiments 1 or 3-82, wherein E¹ is CH or CR⁶, wherein R⁶ is halo (e.g., —F, or —Cl).

Embodiment 84. The compound of Embodiment 1, wherein the compound is a compound of Formula (Aa):

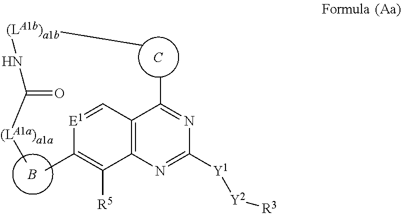

or a pharmaceutically acceptable salt thereof, wherein:
Ring C is a 4-10 membered heterocyclylene optionally substituted with 1-4 R⁷, wherein Ring C has one ring nitrogen atom, 0-1 ring oxygen atom, and no additional ring heteroatoms;
Ring B is selected from the group consisting of: C₆₋₁₀ arylene and 5-10 membered heteroarylene, each of which is optionally substituted with 1-3 substituents independently selected from the group consisting of: R^{a} and R^{b}.
a1a and a1b are independently integers from 0 to 5, provided that a1a+a1b is 4, 5, or 6;
each L^{A1a} and L^{A1b} is an independently selected L^{A1};
R³ is a 5-8 membered heterocyclyl optionally substituted with 1-3 R^{a}; and
R⁵ is H or halo.

Embodiment 85. The compound of Embodiment 84, wherein:
E¹ is N, CH, or C-halo;
Ring C is selected from the group consisting of:

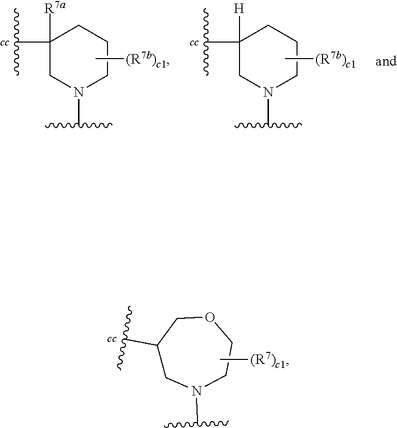

wherein:
c1 is 0, 1, or 2;
cc represents the point of attachment to -(L^{A1b})_{a1b}-;
each R^{7a} and R^{7b} is an independently selected R⁷; and
Ring B is selected from the group consisting of:

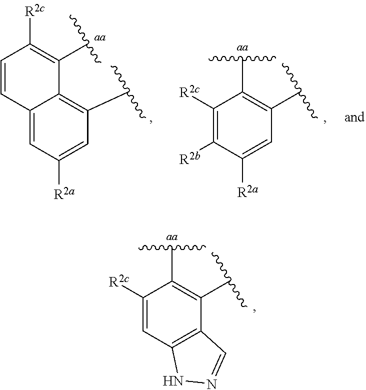

wherein R^{2a} is —OH or —NH₂; R^{2b} and R^{2c} are independently H or halo (e.g., —Cl); and aa represents the point of attachment to -(L^{A1a})ₐ₁ₐ-.

Embodiment 86. The compound of Embodiments 1 or 2, wherein the compound is a compound of Formula (I-c) or (I-cc):

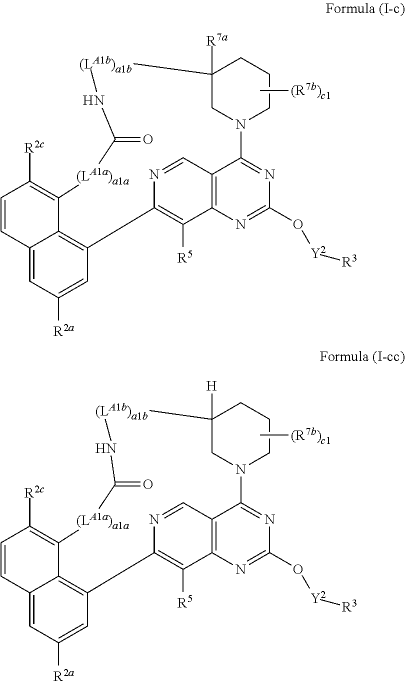

or a pharmaceutically acceptable salt thereof, wherein:
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2a} and R^{2c} are independently H or R^{a};
Y² is —CH₂— or

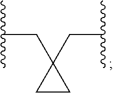

R³ is a 5-8 membered heterocyclyl optionally substituted with 1-3 R^{a}; and
R⁵ is H or halo.

Embodiment 87. The compound of Embodiments 1 or 3, wherein the compound is a compound of Formula (II-c) or (II-cc):

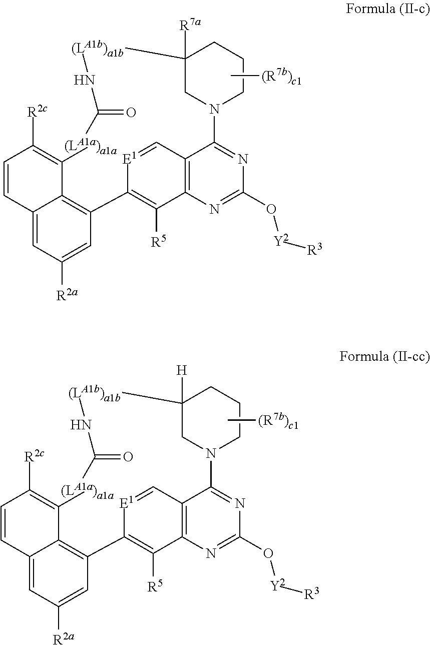

or a pharmaceutically acceptable salt thereof, wherein:
E¹ is CH or CR⁶;
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2a} and R^{2c} are independently H or R^{a};
Y² is —CH₂— or

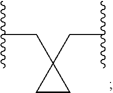

R³ is a 5-8 membered heterocyclyl optionally substituted with 1-3 R^{a}; and
R⁵ is H or halo.

Embodiment 88. The compound of Embodiments 1 or 2, wherein the compound is a compound of Formula (I-d) or (I-dd):

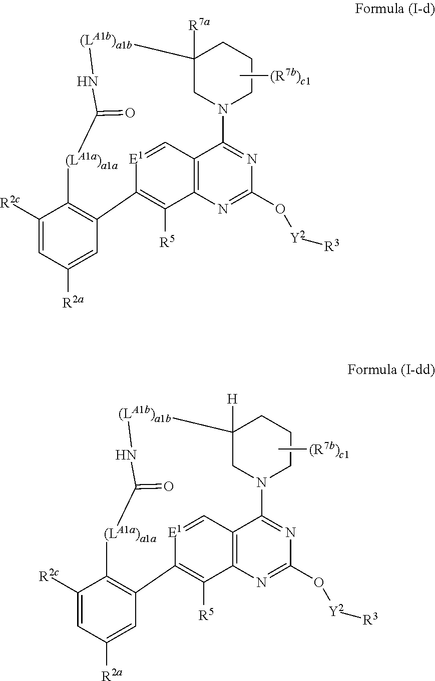

or a pharmaceutically acceptable salt thereof, wherein.
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1}.
R^{2a} and R^{2c} are independently R^{a};
Y² is —CH₂— or

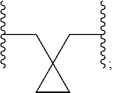 [SMILES: *CC1(C*)CC1]

R³ is a 5-8 membered heterocyclyl optionally substituted with 1-3 R^{a}; and
R⁵ is H or halo.

Embodiment 89. The compound of Embodiments 1 or 3, wherein the compound is a compound of Formula (II-d) or (II-dd):

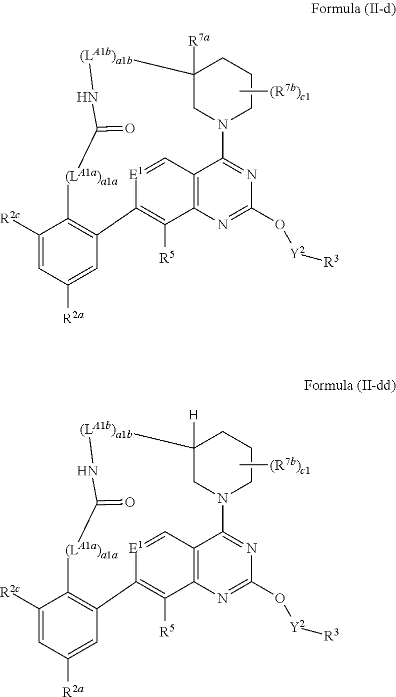

or a pharmaceutically acceptable salt thereof, wherein:
E¹ is CH or CR⁶;
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2a} and R^{2c} are independently R^{a};
Y² is —CH₂— or

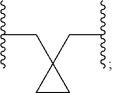 [SMILES: *CC1(C*)CC1]

R³ is a 5-8 membered heterocyclyl optionally substituted with 1-3 R^{a}; and
R⁵ is H or halo.

Embodiment 90. The compound of Embodiments 1 or 2, wherein the compound is a compound of Formula (I-e) or (I-ee):

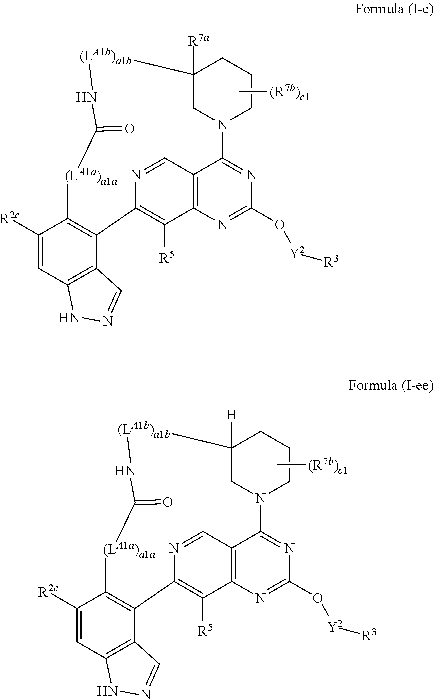

or a pharmaceutically acceptable salt thereof, wherein:
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2c} is H or R^{a};
Y² is —CH₂— or

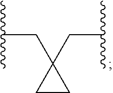 [SMILES: *CC1(C*)CC1]

R³ is a 5-8 membered heterocyclyl optionally substituted with 1-3 R^{a}; and
R⁵ is H or halo.

Embodiment 91. The compound of Embodiments 1 or 3, wherein the compound is a compound of Formula (II-e) or (II-ee):

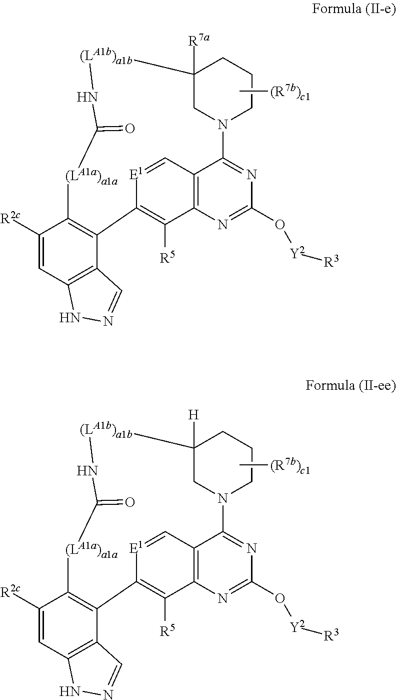

or a pharmaceutically acceptable salt thereof, wherein:
E¹ is CH or CR⁶;
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2c} is H or R^{a};
Y² is —CH₂— or

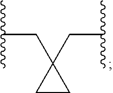 [SMILES: *CC1(C*)CC1]

R³ is a 5-8 membered heterocyclyl optionally substituted with 1-3 R^{a}; and
R⁵ is H or halo.

Embodiment 92. The compound of Embodiments 1 or 2, wherein the compound is a compound of Formula (I-f):

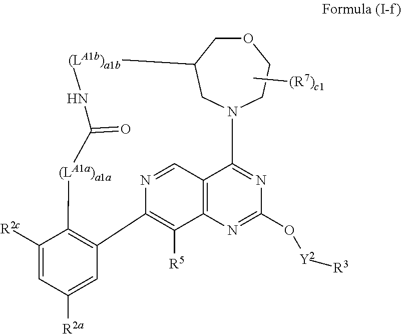

or a pharmaceutically acceptable salt thereof, wherein:
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2a} and R^{2c} are independently R^{a};
Y² is —CH₂— or

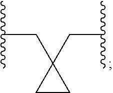 [SMILES: *CC1(C*)CC1]

R³ is a 5-8 membered heterocyclyl optionally substituted with 1-3 R^{a}; and
R⁵ is H or halo.

Embodiment 93. The compound of Embodiments 1 or 3, wherein the compound is a compound of Formula (II-f):

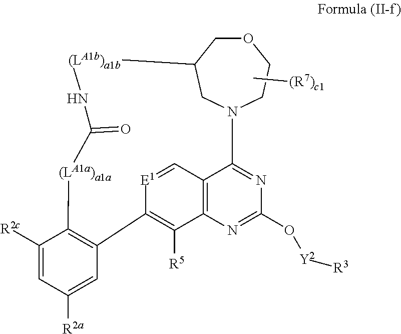

or a pharmaceutically acceptable salt thereof, wherein:
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2a} and R^{2c} are independently R^{a};
Y² is —CH₂— or

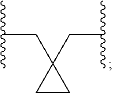 [SMILES: *CC1(C*)CC1]

R³ is a 5-8 membered heterocyclyl optionally substituted with 1-3 R^{a}; and
R⁵ is H or halo.

Embodiment 94. The compound of Embodiments 1 or 2, wherein the compound is a compound of Formula (I-g):

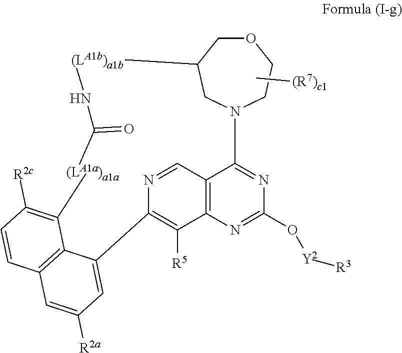

or a pharmaceutically acceptable salt thereof, wherein:
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2a} and R^{2c} are independently H or R^{a};
Y² is —CH₂— or

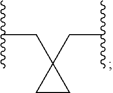 [SMILES: *CC1(C*)CC1]

R³ is a 5-8 membered heterocyclyl optionally substituted with 1-3 R^{a}; and
R⁵ is H or halo.

Embodiment 95. The compound of Embodiments 1 or 3, wherein the compound is a compound of Formula (II-g):

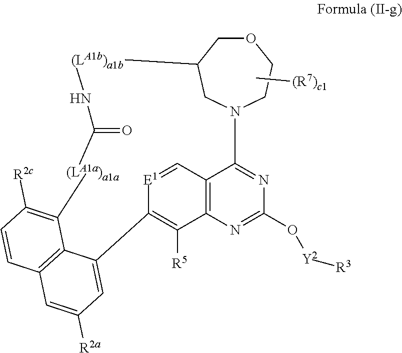

or a pharmaceutically acceptable salt thereof, wherein:
E¹ is CH or CR⁶;
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2a} and R^{2c} are independently H or R^{a};
Y² is —CH₂— or

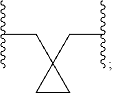 [SMILES: *CC1(C*)CC1]

R³ is a 5-8 membered heterocyclyl optionally substituted with 1-3 R^{a}; and
R⁵ is H or halo.

Embodiment 96. The compound of Embodiments 1 or 2, wherein the compound is a compound of Formula (I-h):

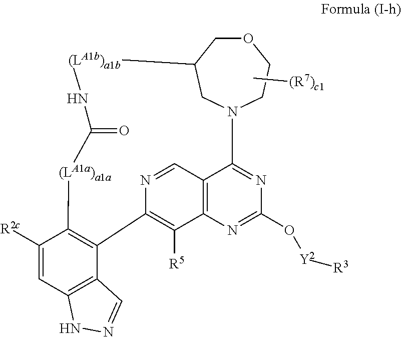

or a pharmaceutically acceptable salt thereof, wherein:
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2c} is H or R^{a};
Y² is —CH₂— or

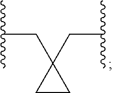 [SMILES: *CC1(C*)CC1]

R³ is a 5-8 membered heterocyclyl optionally substituted with 1-3 R^{a}; and
R⁵ is H or halo.

Embodiment 97. The compound of Embodiments 1 or 3, wherein the compound is a compound of Formula (II-h):

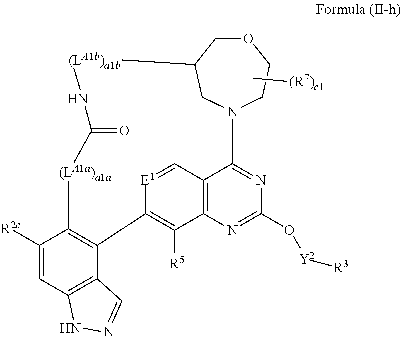

or a pharmaceutically acceptable salt thereof, wherein:
E¹ is CH or CR⁶;
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2c} is H or R^{a};
Y² is —CH₂— or

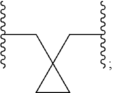 [SMILES: *CC1(C*)CC1]

R³ is a 5-8 membered heterocyclyl optionally substituted with 1-3 R^{a}; and
R⁵ is H or halo.

Embodiment 98. The compound of any one of Embodiments 84-97, wherein Y² is CH₂; R³ is

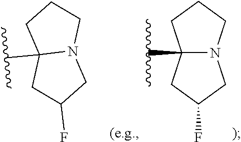 [SMILES: *C12CCCN1CC(F)C2 ; *[C@@]12CCCN1C[C@H](F)C2]

### and R⁵ is F.

Embodiment 99. The compound of any one of Embodiments 85-89, 92-95, or 98, wherein R^{2a} is —OH.

Embodiment 100. The compound of any one of Embodiments 85-89, 92-95, or 98, wherein R^{2a} is —NH₂.

Embodiment 101. The compound of any one of Embodiments 85-100, wherein R^{2c} is —F.

Embodiment 102. The compound of any one of Embodiments 85-100, wherein R^{2c} is —Cl.

Embodiment 103. The compound of any one of Embodiments 85-102, wherein c1 is 0.

Embodiment 104. The compound of any one of Embodiments 85-91 or 98-103, wherein R^{7a}, when present, is OH.

Embodiment 105. The compound of Embodiments 86 or 87, wherein: R^{7a}, when present, is OH; c1 is 0; R^{2a} is —OH; and R^{2c} is H or —F.

Embodiment 106. The compound of any one of Embodiments 84-105, wherein a1b is 2; and a1a is 2, 3, or 4.

Embodiment 107. The compound of any one of Embodiments 84-105, wherein a1b is 1; and a1a is 3, 4, or 5.

Embodiment 108. The compound of any one of Embodiments 84-107, wherein each of L^{A1a} and L^{A1b} is CH₂.

Embodiment 109. The compound of any one of Embodiments 84-108, wherein R⁶ is —F or —Cl; and R⁵ is —F.

Embodiment 110. The compound of Embodiment 1, wherein the compound is selected from the group consisting of Compound No. 101, 101a, 102, 102a, 103, 103a, 104, 104a, 105, 105a, 106, 106a, 107, 107a, 108, 108a, 109, 109a, 110, 110a, 111, 111a, 112, 112a, 113, 113a, 114, 114a, 115, 115a, 115b, 115c, 116, 116a, 117, 117a, 118, 118a, 119, 119a, 120, 120a, 121, 121a, 122, 122a, 123, 123a, 124, 124a, 125, 125a, 126, 126a, 127, 127a, 128, 128a, 129, 129a, 130, 130a, 131, 131a, 132, 132a, 133, 133a, 134, 134a, 135, 135a, 136, 136a, 137, 137a, 138, 138a, 139, 139a, 140, 140a, 140b, 140c, 141, 141a, 143, 143a, 151, 151a, 152, 152a, 153, 153a, 154, 154a, 155, 155a, 156, 156a, 157, 157a, 158, 158a, 159, 159a, 159b, 159c, 160, 160a, 161, 161a, 162, 162a, 163, 163a, 164, 164a, 165, 165a, 166, 166a, 167, 167a, 170, 170a, 171, 171a, 172, 172a, 173, 173a, 174, 174a, 175, 175a, 176, 176a, 177, 178, 179, 179a, 180, 180a, 181, 181a, 182, 182a, 183, 183a, 184, 184a, 185, 185a, 186, 186a, 187, 187a, 188, 188a, 188b, 189, 189a, 190, 190a, 191, 191a, 192, 192a, 193, 193a, 194, 194a, 195, 195a, 196, 196a, 197, 197a, 197b, 198, 198a, 199, 199a, 200, 200a, 201, 201a, 202, 202a, 202b, 203, and 203a, as depicted in in Table C1, or a pharmaceutically acceptable salt thereof.

Embodiment 111. A pharmaceutical composition comprising a compound of any one of Embodiments 1-110, or a pharmaceutically acceptable salt thereof, and a pharmaceutically acceptable excipient.

Embodiment 112. A dysregulated KRas protein non-covalently bound with a compound of any one of Embodiments 1-110, or a pharmaceutically acceptable salt thereof.

Embodiment 113. A method for treating a KRas-associated cancer in a subject in need thereof, the method comprising administering to the subject a therapeutically effective amount of a compound of any one of Embodiments 1-110, or a pharmaceutically acceptable salt thereof, or a pharmaceutical composition according to Embodiment 111.

Embodiment 114. A method for treating a KRas-associated cancer in a subject in need thereof, the method comprising (a) determining that the cancer in the subject has a KRas dysregulation; and (b) administering to the subject a therapeutically effective amount of a compound of any one of Embodiments 1-110, or a pharmaceutically acceptable salt thereof, or a pharmaceutical composition according to Embodiment 111.

Embodiment 115. A method of treating a KRas-associated cancer in a subject, the method administering to a subject identified or diagnosed as having a cancer having a KRas dysregulation a therapeutically effective amount of a compound of any one of Embodiments 1-110 or a pharmaceutically acceptable salt thereof, or a pharmaceutical composition according to Embodiment 111.

Embodiment 116. A method of treating a KRas-associated cancer in a subject, the method comprising:
(a) determining that the cancer in the subject has a KRas dysregulation; and
(b) administering to the subject a therapeutically effective amount of a compound of any one of Embodiments 1-110 or a pharmaceutically acceptable salt thereof, or a pharmaceutical composition according to Embodiment 111.

Embodiment 117. The method of any one of Embodiments 113-116, wherein the KRas-associated cancer is a mutant KRas-associated cancer.

Embodiment 118. The method of Embodiment 117, wherein the mutant KRas-associated cancer is a KRas G12A-associated cancer, a KRas G12C-associated cancer, a KRas G12D-associated cancer, a KRas G12R-associated cancer, a KRas G12S-associated cancer, or a KRas G12V-associated cancer.

Embodiment 119. The method of Embodiment 118, wherein the mutant KRas-associated cancer is a KRas G12D-associated cancer or a KRas G12V-associated cancer.

Embodiment 120. The method of Embodiment 118, wherein the mutant KRas-associated cancer is a KRas G12D-associated cancer.

Embodiment 121. The method of Embodiment 118, wherein the mutant KRas-associated cancer is a KRas G12R-associated cancer.

Embodiment 122. The method of Embodiment 118, wherein the mutant KRas-associated cancer is a KRas G12V-associated cancer.

Embodiment 123. The method of any one of Embodiments 114 or 116, wherein the step of determining that the cancer in the subject has a KRas dysregulation includes performing an assay to detect the KRas dysregulation (e.g., a KRas mutation) in a tumor sample from the subject.

Embodiment 124. The method of Embodiment 123, wherein detecting the KRas dysregulation includes detecting a KRAS gene having a mutation corresponding to a substitution of glycine 12 in a KRas protein and/or a KRas protein having a substitution of glycine 12.

Embodiment 125. The method of Embodiment 124, wherein the substitution of glycine 12 is a substitution to alanine, cysteine, aspartic acid, arginine, serine, or valine.

Embodiment 126. The method of Embodiment 125, wherein the substitution of glycine 12 is a substitution to aspartic acid.

Embodiment 127. The method of Embodiment 125, wherein the substitution of glycine 12 is a substitution to arginine.

Embodiment 128. The method of Embodiment 125, wherein the substitution of glycine 12 is a substitution to valine.

Embodiment 129. The method of any one of Embodiments 123-128, comprising obtaining a tumor sample from the subject.

Embodiment 130. The method of Embodiment 129, wherein the tumor sample is a biopsy sample.

Embodiment 131. The method of any one of Embodiments 123-130, wherein the assay is selected from the group consisting of sequencing, immunohistochemistry, and enzyme-linked immunosorbent assay, and fluorescence in situ hybridization (FISH).

Embodiment 132. The method of Embodiment 131, wherein the sequencing is pyrosequencing or next generation sequencing.

Embodiment 133. The method of any one of Embodiments 113-132, wherein the KRas-associated cancer is selected from the group consisting of: a hematological cancer, a soft tissue cancer, bile duct cancer, bladder cancer, brain cancer, breast cancer, cervical cancer, colon cancer, endometrial cancer, esophageal cancer, kidney cancer, liver cancer, lung cancer, mucinous carcinoma, ovarian cancer, pancreatic cancer, prostate cancer, rectal cancer, skin cancer, stomach cancer, testicular cancer, thymus cancer, thyroid cancer, urothelial cancer, uterine cancer, and a combination thereof.

Embodiment 134. The method of Embodiment 133, wherein the KRas-associated cancer is pancreatic cancer.

Embodiment 135. The method of any one of Embodiments 113-134, comprising administering an additional therapy or therapeutic agent to the subject.

Embodiment 136. The method of Embodiment 135, wherein the additional therapy or therapeutic agent is selected from the group consisting of Ras pathway targeted therapeutic agents, kinase-targeted therapeutics, mTORC1 inhibitors or degraders, YAP inhibitors or degraders, proteasome inhibitors or degraders, HSP90 inhibitors or degraders, farnesyl transferase inhibitors or degraders, PTEN inhibitors or degraders, signal transduction pathway inhibitors or degraders, checkpoint inhibitors, modulators of the apoptosis pathway, chemotherapeutics, angiogenesis-targeted therapies, immune-targeted agents, radiotherapy, and combinations thereof.

Embodiment 137. A method for a method for modulating KRas protein activity in a mammalian cell, the method comprising contacting the mammalian cell with an effective amount of a compound of any one of Embodiments 1-110, or a pharmaceutically acceptable salt thereof.

Embodiment 138. The method of Embodiment 137, wherein the contacting occurs in vivo.

Embodiment 139. The method of Embodiment 137, wherein the contacting occurs in vitro.

Embodiment 140. The method of Embodiment 137, wherein the contacting occurs ex vivo.

Embodiment 141. The method of any one of Embodiments 137-140, wherein the mammalian cell is a mammalian cancer cell.

Embodiment 142. The method of any one of Embodiments 137-141, wherein the KRas protein is a mutant KRas protein.

Embodiment 143. The method of Embodiment 142, wherein the mutant KRas protein is a mutant KRas protein selected from the group consisting of: a KRas G12A mutant protein, a KRas G12C mutant protein, a KRas G12D mutant protein, a KRas G12R mutant protein, a KRas G12S mutant protein, and a KRas G12V mutant protein.

### Exemplary Formula I-ee Embodiments

Embodiment 1. A compound of Formula (I-ee):

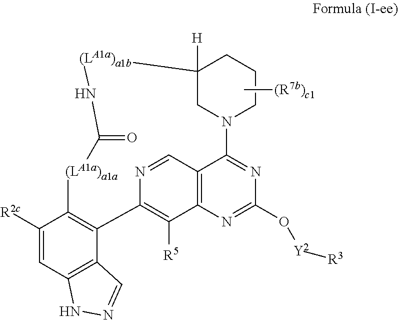

or a pharmaceutically acceptable salt thereof, wherein:
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
each L^{A1} is independently selected from the group consisting of: —CH₂—, —CHR^{L}—, and —C(R^{L})₂—, wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, and C₁₋₆ alkyl optionally substituted with 1-6 R^{c};
R^{2c} is H or R^{a};
Y² is —CH₂— or

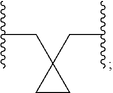 [SMILES: *CC1(C*)CC1]

R³ is:
a 5-8 membered heterocyclyl optionally substituted with 1-3 R^{a}, or
—NR^{d}R^{e} (e.g., —NMe₂); and
R⁵ is H or halo;
each R⁷ is independently selected from the group consisting of R^{a} and R^{b};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
b is 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, cyano, —OH, —C₁₋₆ alkoxy, —C₁₋₆ haloalkoxy, —NH₂, —N(H)(C₁₋₃ alkyl), and —N(C₁₋₃ alkyl)₂.

Embodiment 2. The compound of Embodiment 1, wherein Y² is CH₂; R³ is

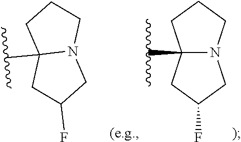 [SMILES: *C12CCCN1CC(F)C2 ; *[C@@]12CCCN1C[C@H](F)C2]

### and R⁵ is F.

Embodiment 3. The compound of Embodiments 1 or 2, wherein R^{2c} is —F.

Embodiment 4. The compound of any one of Embodiments 1-3, wherein R^{2c} is —Cl.

Embodiment 5. The compound of any one of Embodiments 1-4, wherein c1 is 0.

Embodiment 6. The compound of any one of Embodiments 1-5, wherein R^{7a}, when present, is OH.

Embodiment 7. The compound of any one of Embodiments 1-6, wherein a1b is 2; and a1a is 2, 3, or 4.

Embodiment 8. The compound of any one of Embodiments 1-6, wherein a1b is 1; and a1a is 3, 4, or 5.

Embodiment 9. The compound of any one of Embodiments 1-8, wherein each of L^{A1a} and L^{A1b} is CH₂.

Embodiment 10. The compound of any one of Embodiments 1-9, wherein R⁶ is —F or —Cl; and R⁵ is —F.

Embodiment 11. The compound of Embodiment 1, wherein the compound is selected from the group consisting of Compound No. 167, 167a, 196, and 196a, as depicted in Table C1, or a pharmaceutically acceptable salt thereof.

Embodiment 12. A pharmaceutical composition comprising a compound as claimed in any one of Embodiments 1-11, or a pharmaceutically acceptable salt thereof, and a pharmaceutically acceptable excipient.

Embodiment 13. A compound of any one of Embodiments 1-11, or a pharmaceutically acceptable salt thereof, or the pharmaceutical composition of Embodiment 12 for use in treatment.

Embodiment 14. A compound of any one of Embodiments 1-11 or a pharmaceutically acceptable salt thereof, or the pharmaceutical composition of Embodiment 12 for use in the treatment of a cancer selected from the group consisting of a hematological cancer, a soft tissue cancer, bile duct cancer, bladder cancer, brain cancer, breast cancer, cervical cancer, colon cancer, endometrial cancer, esophageal cancer, kidney cancer, liver cancer, lung cancer, mucinous carcinoma, ovarian cancer, pancreatic cancer, prostate cancer, rectal cancer, skin cancer, stomach cancer, testicular cancer, thymus cancer, thyroid cancer, urothelial cancer, and uterine cancer; optionally wherein the cancer is pancreatic cancer.

Embodiment 15. A compound of any one of Embodiments 1-11 or a pharmaceutically acceptable salt thereof, or the pharmaceutical composition of Embodiment 12 for use in the treatment of a KRas-associated cancer in a subject.

Embodiment 16. The compound or pharmaceutical composition of Embodiment 15, wherein the KRas-associated cancer is a mutant KRas-associated cancer.

Embodiment 17. The compound or pharmaceutical composition of Embodiment 16, wherein the mutant KRas-associated cancer is a KRas G12A-associated cancer, a KRas G12C-associated cancer, a KRas G12D-associated cancer, a KRas G12R-associated cancer, a KRas G12S-associated cancer, or a KRas G12V-associated cancer; optionally wherein the mutant KRas-associated cancer is a KRas G12D-associated cancer or a KRas G12V-associated cancer; optionally wherein the mutant KRas-associated cancer is a KRas G12D-associated cancer; optionally wherein the mutant KRas-associated cancer is a KRas G12R-associated cancer; or optionally wherein the mutant KRas-associated cancer is a KRas G12V-associated cancer.

### Exemplary Formula II-ee Embodiments

Embodiment 1. A compound of Formula (II-ee):

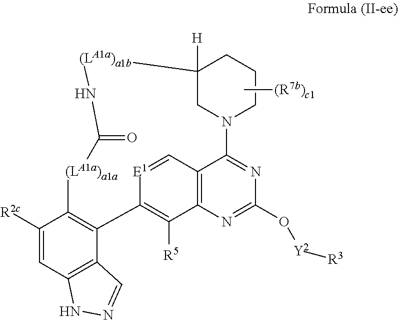

or a pharmaceutically acceptable salt thereof, wherein:
E¹ is CH or CR⁶;
c1 is 0, 1, or 2;
each R^{7b} is an independently selected R⁷;
a1a and a1b are independently integers from 0 to 5, provided that a1a+a1b is 4, 5, or 6;
each L^{A1a} and L^{A1b} is an independently selected L^{A1};
each L^{A1} is independently selected from the group consisting of: —CH₂—, —CHR^{L}—, and —C(R^{L})₂—, wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, and C₁₋₆ alkyl optionally substituted with 1-6 R^{c};
R^{2c} is H or R^{a};
Y² is —CH₂— or

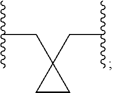 [SMILES: *CC1(C*)CC1]

R³ is:
a 5-8 membered heterocyclyl optionally substituted with 1-3 R^{a}, or
—NR^{d}R^{e} (e.g., —NMe₂);
R⁵ is H or halo;
R⁶ is independently selected from the group consisting of:
(a) —H;
(b) halo;
(c) cyano;
(d) C₁₋₆ alkyl;
(e) C₁₋₆ haloalkyl;
(f) C₁₋₆ alkoxy;
(g) C₁₋₆ haloalkoxy;
(h) —(C₀₋₃ alkylene)-C₃₋₆ cycloalkyl, wherein the C₃₋₆ cycloalkyl portion is optionally substituted with 1-3 R^{g}; and
(i) —O—(C₀₋₃ alkylene)-C₃₋₆ cycloalkyl, wherein the C₃₋₆ cycloalkyl portion is optionally substituted with 1-3 R^{g};
each R⁷ is independently selected from the group consisting of R^{a} and R^{b};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and -R^{b1}, wherein:
b is 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, cyano, —OH, —C₁₋₆ alkoxy, —C₁₋₆ haloalkoxy, —NH₂, —N(H)(C₁₋₃ alkyl), and —N(C₁₋₃ alkyl)₂.

Embodiment 2. The compound of Embodiment 1, wherein Y² is CH₂; R³ is

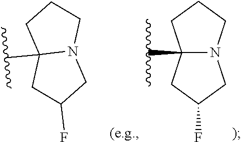 [SMILES: *C12CCCN1CC(F)C2 ; *[C@@]12CCCN1C[C@H](F)C2]

### and R⁵ is F.

Embodiment 3. The compound of Embodiments 1 or 2, wherein R^{2c} is —F.

Embodiment 4. The compound of any one of Embodiments 1-3, wherein R^{2c} is —Cl.

Embodiment 5. The compound of any one of Embodiments 1-4, wherein c1 is 0.

Embodiment 6. The compound of any one of Embodiments 1-5, wherein R^{7a}, when present, is OH.

Embodiment 7. The compound of any one of Embodiments 1-6, wherein a1b is 2; and a1a is 2, 3, or 4.

Embodiment 8. The compound of any one of Embodiments 1-6, wherein a1b is 1; and a1a is 3, 4, or 5.

Embodiment 9. The compound of any one of Embodiments 1-8, wherein each of L^{A1a} and L^{A1b} is CH₂.

Embodiment 10. The compound of any one of Embodiments 1-9, wherein R⁶ is —F or —Cl; and R⁵ is —F.

Embodiment 11. The compound of Embodiment 1, wherein the compound is selected from the group consisting of Compound No. 166, 166a, 197, 197a, 197b, 202, 202a, and 202b as depicted in Table C1, or a pharmaceutically acceptable salt thereof.

Embodiment 12. A pharmaceutical composition comprising a compound as claimed in any one of Embodiments 1-11, or a pharmaceutically acceptable salt thereof, and a pharmaceutically acceptable excipient.

Embodiment 13. A compound of any one of Embodiments 1-11, or a pharmaceutically acceptable salt thereof, or the pharmaceutical composition of Embodiment 12 for use in treatment.

Embodiment 14. A compound of any one of Embodiments 1-11 or a pharmaceutically acceptable salt thereof, or the pharmaceutical composition of Embodiment 12 for use in the treatment of a cancer selected from the group consisting of a hematological cancer, a soft tissue cancer, bile duct cancer, bladder cancer, brain cancer, breast cancer, cervical cancer, colon cancer, endometrial cancer, esophageal cancer, kidney cancer, liver cancer, lung cancer, mucinous carcinoma, ovarian cancer, pancreatic cancer, prostate cancer, rectal cancer, skin cancer, stomach cancer, testicular cancer, thymus cancer, thyroid cancer, urothelial cancer, and uterine cancer; optionally wherein the cancer is pancreatic cancer.

Embodiment 15. A compound of any one of Embodiments 1-11 or a pharmaceutically acceptable salt thereof, or the pharmaceutical composition of Embodiment 12 for use in the treatment of a KRas-associated cancer in a subject.

Embodiment 16. The compound or pharmaceutical composition of Embodiment 15, wherein the KRas-associated cancer is a mutant KRas-associated cancer.

Embodiment 17. The compound or pharmaceutical composition of Embodiment 16, wherein the mutant KRas-associated cancer is a KRas G12A-associated cancer, a KRas G12C-associated cancer, a KRas G12D-associated cancer, a KRas G12R-associated cancer, a KRas G12S-associated cancer, or a KRas G12V-associated cancer; optionally wherein the mutant KRas-associated cancer is a KRas G12D-associated cancer or a KRas G12V-associated cancer; optionally wherein the mutant KRas-associated cancer is a KRas G12D-associated cancer; optionally wherein the mutant KRas-associated cancer is a KRas G12R-associated cancer; or optionally wherein the mutant KRas-associated cancer is a KRas G12V-associated cancer.

### Exemplary Formula I-h Embodiments

Embodiment 1. A compound of Formula (I-h):

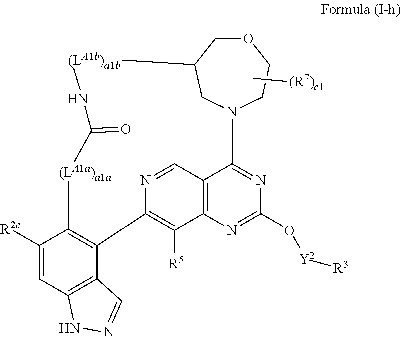

or a pharmaceutically acceptable salt thereof, wherein:
c1 is 0, 1, or 2;
a1a and a1b are independently integers from 0 to 5, provided that a1a+a1b is 4, 5, or 6;
each L^{A1a} and L^{A1b} is an independently selected L^{A1};
each L^{A1} is independently selected from the group consisting of: —CH₂—, —CHR^{L}—, and —C(R^{L})₂—, wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, and C₁₋₆ alkyl optionally substituted with 1-6 R^{c};
R^{2c} is H or R^{a};
Y² is —CH₂— or

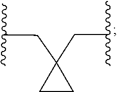 [SMILES: *CC1(C*)CC1]

R³ is:
a 5-8 membered heterocyclyl optionally substituted with 1-3 R^{a}, or
—NR^{d}R^{e} (e.g., —NMe₂);
R⁵ is H or halo;
each R⁷ is independently selected from the group consisting of R^{a} and R^{b};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
b is 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, cyano, —OH, —C₁₋₆ alkoxy, —C₁₋₆ haloalkoxy, —NH₂, —N(H)(C₁₋₃ alkyl), and —N(C₁₋₃ alkyl)₂.

Embodiment 2. The compound of Embodiment 1, wherein Y² is CH₂; R³ is

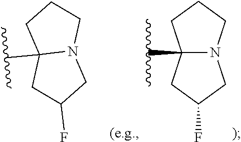 [SMILES: *C12CCCN1CC(F)C2 ; *[C@@]12CCCN1C[C@H](F)C2]

### and R⁵ is F.

Embodiment 3. The compound of Embodiments 1 or 2, wherein R^{2c} is —F.

Embodiment 4. The compound of any one of Embodiments 1-3, wherein R^{2c} is —Cl.

Embodiment 5. The compound of any one of Embodiments 1-4, wherein c1 is 0.

Embodiment 6. The compound of any one of Embodiments 1-5, wherein R^{7a}, when present, is OH.

Embodiment 7. The compound of any one of Embodiments 1-6, wherein a1b is 2; and a1a is 2, 3, or 4.

Embodiment 8. The compound of any one of Embodiments 1-6, wherein a1b is 1; and a1a is 3, 4, or 5.

Embodiment 9. The compound of any one of Embodiments 1-8, wherein each of L^{A1a} and L^{A1b} is CH₂.

Embodiment 10. The compound of any one of Embodiments 1-9, wherein R⁶ is —F or —Cl; and R⁵ is —F.

Embodiment 11. The compound of Embodiment 1, wherein the compound is selected from the group consisting of Compound Nos. 203 and 203a as depicted in Table C1, or a pharmaceutically acceptable salt thereof.

Embodiment 12. A pharmaceutical composition comprising a compound as claimed in any one of Embodiments 1-11, or a pharmaceutically acceptable salt thereof, and a pharmaceutically acceptable excipient.

Embodiment 13. A compound of any one of Embodiments 1-11, or a pharmaceutically acceptable salt thereof, or the pharmaceutical composition of Embodiment 12 for use in treatment.

Embodiment 14. A compound of any one of Embodiments 1-11 or a pharmaceutically acceptable salt thereof, or the pharmaceutical composition of Embodiment 12 for use in the treatment of a cancer selected from the group consisting of a hematological cancer, a soft tissue cancer, bile duct cancer, bladder cancer, brain cancer, breast cancer, cervical cancer, colon cancer, endometrial cancer, esophageal cancer, kidney cancer, liver cancer, lung cancer, mucinous carcinoma, ovarian cancer, pancreatic cancer, prostate cancer, rectal cancer, skin cancer, stomach cancer, testicular cancer, thymus cancer, thyroid cancer, urothelial cancer, and uterine cancer; optionally wherein the cancer is pancreatic cancer.

Embodiment 15. A compound of any one of Embodiments 1-11 or a pharmaceutically acceptable salt thereof, or the pharmaceutical composition of Embodiment 12 for use in the treatment of a KRas-associated cancer in a subject.

Embodiment 16. The compound or pharmaceutical composition of Embodiment 15, wherein the KRas-associated cancer is a mutant KRas-associated cancer.

Embodiment 17. The compound or pharmaceutical composition of Embodiment 16, wherein the mutant KRas-associated cancer is a KRas G12A-associated cancer, a KRas G12C-associated cancer, a KRas G12D-associated cancer, a KRas G12R-associated cancer, a KRas G12S-associated cancer, or a KRas G12V-associated cancer; optionally wherein the mutant KRas-associated cancer is a KRas G12D-associated cancer or a KRas G12V-associated cancer; optionally wherein the mutant KRas-associated cancer is a KRas G12D-associated cancer; optionally wherein the mutant KRas-associated cancer is a KRas G12R-associated cancer; or optionally wherein the mutant KRas-associated cancer is a KRas G12V-associated cancer.

### Exemplary Formula II-h Embodiments

Embodiment 1. A compound of Formula (II-h):

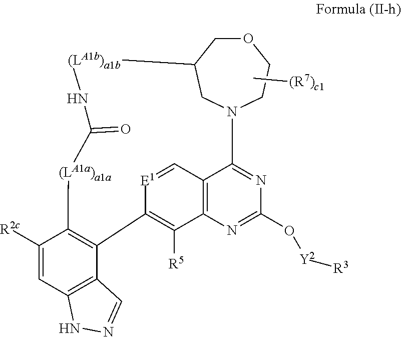

or a pharmaceutically acceptable salt thereof, wherein:
E1 is CH or CR⁶;
c1 is 0, 1, or 2;
a1a and a1b are independently integers from 0 to 5, provided that a1a+a1b is 4, 5, or 6;
each L^{A1a} and L^{A1b} is an independently selected L^{A1};
each L^{A1} is independently selected from the group consisting of: —CH₂—, —CHR^{L}—, and —C(R^{L})₂—, wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, and C₁₋₆ alkyl optionally substituted with 1-6 R^{c};
R^{2c} is H or R^{a};
Y² is —CH₂— or

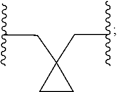 [SMILES: *CC1(C*)CC1]

R³ is:
a 5-8 membered heterocyclyl optionally substituted with 1-3 R^{a}, or
—NR^{d}R^{e} (e.g., —NMe₂);
R⁵ is H or halo;
R⁶ is independently selected from the group consisting of:
(a) —H;
(b) halo;
(c) cyano;
(d) C₁₋₆ alkyl;
(e) C₁₋₆ haloalkyl;
(f) C₁₋₆ alkoxy;
(g) C₁₋₆ haloalkoxy;
(h) —(C₀₋₃ alkylene)-C₃₋₆ cycloalkyl, wherein the C₃₋₆ cycloalkyl portion is optionally substituted with 1-3 R^{g}; and
(i) —O—(C₀₋₃ alkylene)-C₃₋₆ cycloalkyl, wherein the C₃₋₆ cycloalkyl portion is optionally substituted with 1-3 R^{g};
each R⁷ is independently selected from the group consisting of R^{a} and R^{b},
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
b is 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, cyano, —OH, —C₁₋₆ alkoxy, —C₁₋₆ haloalkoxy, —NH₂, —N(H)(C₁₋₃ alkyl), and —N(C₁₋₃ alkyl)₂.

Embodiment 2. The compound of Embodiment 1, wherein Y² is CH₂; R³ is

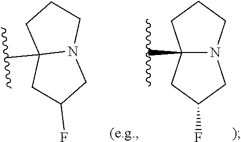 [SMILES: *C12CCCN1CC(F)C2 ; *[C@@]12CCCN1C[C@H](F)C2]

### and R⁵ is F.

Embodiment 3. The compound of Embodiments 1 or 2, wherein R^{2c} is —F.

Embodiment 4. The compound of any one of Embodiments 1-3, wherein R^{2c} is —Cl.

Embodiment 5. The compound of any one of Embodiments 1-4, wherein c1 is 0.

Embodiment 6. The compound of any one of Embodiments 1-5, wherein R^{7a}, when present, is OH.

Embodiment 7. The compound of any one of Embodiments 1-6, wherein a1b is 2; and a1a is 2, 3, or 4.

Embodiment 8. The compound of any one of Embodiments 1-6, wherein a1b is 1; and a1a is 3, 4, or 5.

Embodiment 9. The compound of any one of Embodiments 1-8, wherein each of L^{A1a} and L^{A1b} is CH₂.

Embodiment 10. The compound of any one of Embodiments 1-9, wherein R⁶ is —F or —Cl; and R⁵ is —F.

Embodiment 11. The compound of Embodiment 1, wherein the compound is selected from the group consisting of Compound Nos. 198, 198a, 200, and 200a, as depicted in Table C1, or a pharmaceutically acceptable salt thereof.

Embodiment 12. A pharmaceutical composition comprising a compound as claimed in any one of Embodiments 1-11, or a pharmaceutically acceptable salt thereof, and a pharmaceutically acceptable excipient.

Embodiment 13. A compound of any one of Embodiments 1-11, or a pharmaceutically acceptable salt thereof, or the pharmaceutical composition of Embodiment 12 for use in treatment.

Embodiment 14. A compound of any one of Embodiments 1-11 or a pharmaceutically acceptable salt thereof, or the pharmaceutical composition of Embodiment 12 for use in the treatment of a cancer selected from the group consisting of a hematological cancer, a soft tissue cancer, bile duct cancer, bladder cancer, brain cancer, breast cancer, cervical cancer, colon cancer, endometrial cancer, esophageal cancer, kidney cancer, liver cancer, lung cancer, mucinous carcinoma, ovarian cancer, pancreatic cancer, prostate cancer, rectal cancer, skin cancer, stomach cancer, testicular cancer, thymus cancer, thyroid cancer, urothelial cancer, and uterine cancer; optionally wherein the cancer is pancreatic cancer.

Embodiment 15. A compound of any one of Embodiments 1-11 or a pharmaceutically acceptable salt thereof, or the pharmaceutical composition of Embodiment 12 for use in the treatment of a KRas-associated cancer in a subject.

Embodiment 16. The compound or pharmaceutical composition of Embodiment 15, wherein the KRas-associated cancer is a mutant KRas-associated cancer.

Embodiment 17. The compound or pharmaceutical composition of Embodiment 16, wherein the mutant KRas-associated cancer is a KRas G12A-associated cancer, a KRas G12C-associated cancer, a KRas G12D-associated cancer, a KRas G12R-associated cancer, a KRas G12S-associated cancer, or a KRas G12V-associated cancer; optionally wherein the mutant KRas-associated cancer is a KRas G12D-associated cancer or a KRas G12V-associated cancer; optionally wherein the mutant KRas-associated cancer is a KRas G12D-associated cancer; optionally wherein the mutant KRas-associated cancer is a KRas G12R-associated cancer; or optionally wherein the mutant KRas-associated cancer is a KRas G12V-associated cancer.

What is claimed is:

1. A compound of Formula (A): 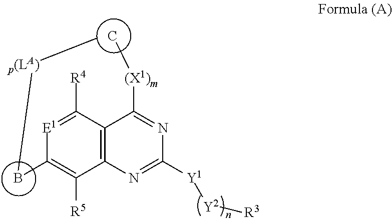
or a pharmaceutically acceptable salt thereof, wherein:
m is 0, 1, or 2;
each X¹ is independently selected from the group consisting of CH₂, CHR^{a}, C(R^{a})₂, O, N(R^{d}), C(═O), and S(O)₀₋₂, provided that —(X¹)ₘ— does not include any O—O, O—N, N—S(O)₀, or O—S(O)₀₋₂ bonds;
Ring C is selected from the group consisting of: C₃₋₁₅ cycloalkylene, 4-15 membered heterocyclylene, 6-15 membered arylene, and 5-15 membered heteroarylene, each of which is optionally substituted with 1-4 R⁷, wherein:
each R⁷ is independently selected from the group consisting of R^{a} and R^{b};
p is an integer from 3 to 10;
each L^{A} is independently selected from the group consisting of: L^{A1}, L^{A2}, L^{A3}, and L^{A4}, provided that 0-2 occurrences of L^{A} are L^{A4}, and 0-3 occurrences of L^{A} are selected from the group consisting of L^{A2} and L^{A3};
each L^{A1} is independently selected from the group consisting of: —CH₂—, —CHR^{L}—, and —C(R^{L})₂—, wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, and C₁₋₆ alkyl optionally substituted with 1-6 R^{c};
each L^{A2} is independently selected from the group consisting of: 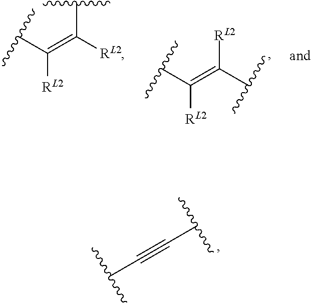
wherein each R^{L2} is independently selected from the group consisting of: H, halo, CN, and C₁₋₆ alkyl optionally substituted with 1-6 R^{c};
each L^{A3} is independently selected from the group consisting of: —N(R^{d})—, —O—, —S(O)₀₋₂—, and C(═O);
each L^{A4} is independently selected from the group consisting of: C₃₋₁₀ cycloalkylene, 4-10 membered heterocyclylene, C₆₋₁₀ arylene, and 5-10 membered heteroarylene, each of which is optionally substituted with 1-3 R^{g};
Ring B is selected from the group consisting of: C₆₋₁₀ arylene and 5-10 membered heteroarylene, each of which is optionally substituted with 1-6 substituents independently selected from the group consisting of: R^{a} and R^{b};
Y¹ is selected from the group consisting of: a bond, —N(H)—, —N(C₁₋₃ alkyl)-, —N(C₃₋₆ cycloalkyl)-, —O—, and —S(O)₀₋₂—;
n is 0 or 1;
Y² is a straight-chain C₁₋₆ alkylene optionally substituted with 1-6 R^{Y};
each R^{Y} is independently selected from the group consisting of: halo, cyano, —OH, oxo, C₁₋₆ alkoxy, C₁₋₆ haloalkoxy, C₁₋₆ alkyl, and C₁₋₆ haloalkyl, or
a pair of R^{Y} on the same or different carbon atom(s) taken together with the atom(s) connecting them form a C₃₋₆ cycloalkyl ring or 4-6 membered heterocyclyl ring, each of which is optionally substituted with 1-3 independently selected C₁₋₃ alkyl;
R³ is selected from the group consisting of:
(a) —H;
(b) -halo;
(c) —NR^{d}R^{e};
(d) C₃₋₁₀ cycloalkyl or 3-15 membered heterocyclyl, each of which is optionally substituted with 1-6 substituents independently selected from the group consisting of: R^{a} and R^{b}; and
(e) C₆₋₁₀ aryl or 5-10 membered heteroaryl, each of which is optionally substituted with 1-6 substituents independently selected from the group consisting of: R^{a} and R^{b},
E¹ is N, CH, or CR⁶;
R⁴, R⁵, and R⁶ are independently selected from the group consisting of:
(a) —H;
(b) halo;
(c) cyano;
(d) C₁₋₆ alkyl;
(e) C₁₋₆ haloalkyl;
(f) C₁₋₆ alkoxy;
(g) C₁₋₆ haloalkoxy;
(h) —(C₀₋₃ alkylene)-C₃₋₆ cycloalkyl, wherein the C₃₋₆ cycloalkyl portion is optionally substituted with 1-3 R^{g}; and
(i) —O—(C₀₋₃ alkylene)-C₃₋₆ cycloalkyl, wherein the C₃₋₆ cycloalkyl portion is optionally substituted with 1-3 R^{g};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{h})_{b}-R^{b1} and —R^{b1}, wherein:
b is 1, 2, or 3;
each -L^{h} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, cyano, —OH, —C₁₋₆ alkoxy, —C₁₋₆ haloalkoxy, —NH₂, —N(H)(C₁₋₃ alkyl), and —N(C₁₋₃ alkyl)₂.

2. The compound of claim 1, wherein the compound of Formula (A) is a compound of Formula (I): 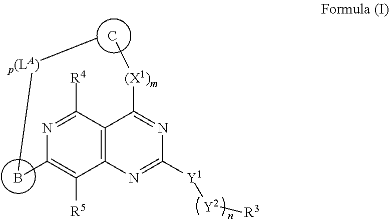
or a pharmaceutically acceptable salt thereof.

3. The compound of claim 1, wherein the compound of Formula (A) is a compound of Formula (II): 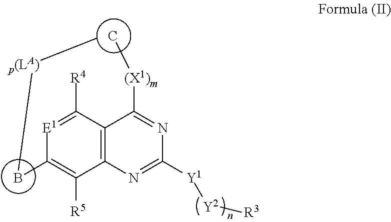
or a pharmaceutically acceptable salt thereof, wherein E¹ is CH or CR⁶.

4. The compound of any one of claims 1-3, wherein m is 0.

5. The compound of any one of claims 1-4, wherein Ring C is a 4-15 membered heterocyclylene optionally substituted with 1-4 R⁷; or
wherein Ring C is a 4-6 membered heterocyclylene optionally substituted with 1-3 R⁷; or
wherein Ring C is a piperidinylene optionally substituted with 1-3 R⁷.

6. The compound of any one of claims 1-5, wherein Ring C is: 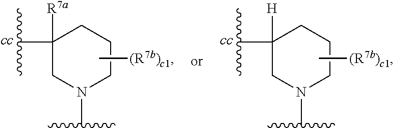
wherein:
c1 is 0, 1, or 2;
each R^{7a} and R^{7b} is an independently selected R⁷; and
cc represents the point of attachment to -(L^{A})ₚ-.

7. The compound of claim 6, wherein c1 is 0.

8. The compound of claim 6 or 7, wherein Ring C is 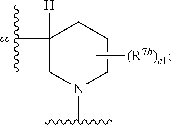
or
wherein Ring C is 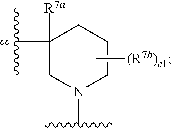
and R^{7a} is C₁₋₃ alkyl optionally substituted with 1-3 F; or
wherein Ring C is 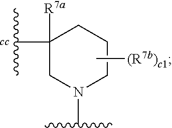
and R^{7a} is —OH.

9. The compound of any one of claims 1-5, wherein Ring C is a 7-10 membered heterocyclylene optionally substituted with 1-3 R⁷.

10. The compound of claim 9, wherein Ring C has one ring nitrogen atom, one ring oxygen atom, and no additional ring heteroatoms.

11. The compound of any one of claims 1-5 or 9-10, wherein Ring C is 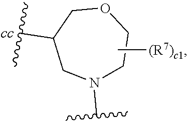
wherein c1 is 0, 1, or 2; and cc represents the point of attachment to -(L^{A})ₚ-.

12. The compound of any one of claims 1-11, wherein p is 4, 5, or 6; or
wherein p is 6, 7, or 8.

13. The compound of any one of claims 1-12, wherein each L^{A} is independently selected from the group consisting of: L^{A1}, L^{A3}, and L^{A4}.

14. The compound of any one of claims 1-13, wherein 0-1 occurrence of L^{A} is L^{A4}; and each remaining L^{A} is independently selected from the group consisting of L^{A1} and L^{A3}.

15. The compound of any one of claims 1-14, wherein one occurrence of L^{A} is L^{A4}; and each remaining L^{A} is independently L^{A1}.

16. The compound of any one of claims 1-14, wherein each L^{A} is independently L^{A1}.

17. The compound of any one of claims 1-14, wherein 1-2 occurrence of L^{A} is independently L^{A3}; and each remaining L^{A} is independently L^{A1}.

18. The compound of any one of claims 1-11, wherein p is 4, 5, or 6; one occurrence of L^{A} is L^{A4}; and each remaining L^{A} is independently L^{A1}.

19. The compound of any one of claims 1-11, wherein p is 6, 7, or 8; and each L^{A} is independently L^{A1}.

20. The compound of claim 19, wherein each L^{A} is —CH₂—.

21. The compound of any one of claims 1-11, wherein p is 6, 7, or 8; one occurrence of L^{A} is C(═O); a second occurrence of L^{A} is N(R^{d}) (e.g., NH); and each remaining L^{A} is independently L^{A1}.

22. The compound of any one of claims 1-11 or 21, wherein -(L^{A})ₚ- is: -(L^{A1a})ₐ₁ₐ-C(═O)NH-(L^{Ab})_{a1b-bb}, wherein:
a1a and a1b are independently integers from 0 to 5, provided that a1a+a1b is 4, 5, or 6;
each L^{A1a} and L^{A1b} is an independently selected L^{A1}; and
bb represents the point of attachment to Ring C.

23. The compound of claim 22, wherein a1b is 2; and a1a is 2, 3, or 4; or
wherein a1b is 1; and a1a is 3, 4, or 5.

24. The compound of any one of claims 22-23, wherein each of L^{A1a} and L^{A1b} is CH₂.

25. The compound of any one of claims 1-3, wherein:
m is 0;
Ring C is: 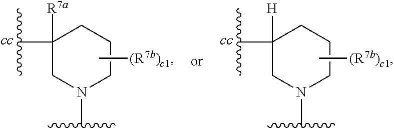
wherein:
c1 is 0, 1, or 2;
each R^{7a} and R^{7b} is an independently selected R⁷; and
cc represents the point of attachment to -(L^{A})ₚ-;
-(L^{A})ₚ- is: -(L^{A1a})ₐ₁ₐ-C(═O)NH-(L^{A1b})_{a1b-bb}, wherein:
a1a and a1b are independently integers from 0 to 5, provided that a1a+a1b is 4, 5, or 6;
each L^{A1a} and L^{A1b} is independently selected L^{A1}; and
bb represents the point of attachment to Ring C.

26. The compound of claim 25, wherein a1b is 2; and a1a is 2, 3, or 4; or
wherein a1b is 1; and a1a is 3, 4, or 5.

27. The compound of claim 25 or 26, wherein each of L^{A1a} and L^{A1b} is CH₂.

28. The compound of any one of claims 23-27, wherein Ring C is 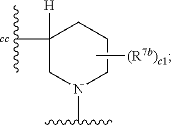
or
wherein Ring C is 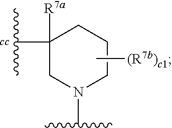
and R^{7a} is —OH.

29. The compound of any one of claims 23-28, wherein c1 is 0.

30. The compound of any one of claims 1-3, wherein:
m is 0;
Ring C is: 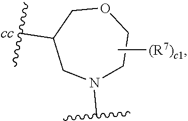
wherein:
c1 is 0, 1, or 2;
cc represents the point of attachment to -(L^{A})ₚ-; and
-(L^{A})ₚ- is: -(L^{A1a})ₐ₁ₐ-C(═O)NH-(L^{A1b})_{a1b-bb}, wherein:
a1a and a1b are independently integers from 0 to 5, provided that a1a+a1b is 4, 5, or 6;
each L^{A1a} and L^{A1b} is independently selected L^{A1}; and
bb represents the point of attachment to Ring C.

31. The compound of claim 30, wherein a1b is 2; and a1a is 2, 3, or 4; or
wherein a1b is 1; and a1a is 3, 4, or 5.

32. The compound of claim 30 or 31, wherein each of L^{A1a} and L^{A1b} is CH₂; and c1 is 0.

33. The compound of any one of claims 1-32, wherein Ring B is a C₆₋₁₀ arylene optionally substituted with 1-6 substituents independently selected from the group consisting of: R^{a} and R^{b}.

34. The compound of any one of claims 1-33, wherein Ring B is naphthylene optionally substituted with 1-4 R^{a}.

35. The compound of any one of claims 1-34, wherein Ring B is 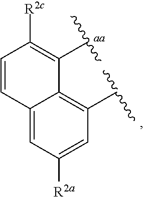
wherein R^{2a} and R^{2c} are independently H or R^{a}; and aa represents the point of attachment to -(L^{A})ₚ-.

36. The compound of claim 35, wherein R^{2a} is —OH; or
wherein R^{2a} is H or —NH₂.

37. The compound of any one of claims 34-36, wherein R^{2c} is halo or H.

38. The compound of any one of claims 1-34, wherein Ring B is 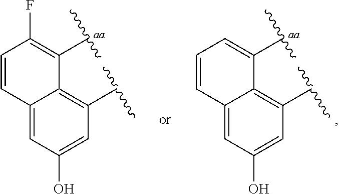
wherein aa represents the point of attachment to -(L^{A})ₚ-.

39. The compound of any one of claims 1-34, wherein Ring B is 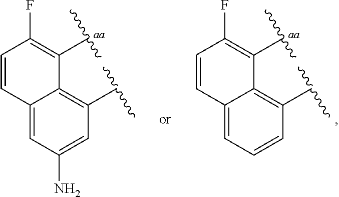
wherein aa represents the point of attachment to -(L^{A})ₚ-.

40. The compound of any one of claims 1-33, wherein Ring B is phenylene optionally substituted with 1-4 R^{a}.

41. The compound of any one of claims 1-33 or 40, wherein Ring B is 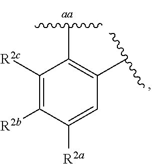
wherein R^{2a}, R^{2b}, and R^{2c} are independently H or R^{a}; and aa represents the point of attachment to -(L^{A})ₚ-; or
wherein Ring B is 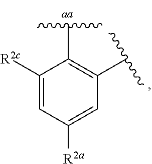
wherein R^{2a} and R^{2c} are independently H or R^{a}; and aa represents the point of attachment to -(L^{A})ₚ-.

42. The compound of claim 41, wherein R^{2a} is —OH; or
wherein R^{2a} is —NH₂.

43. The compound of claim 41 or 42, wherein R^{2c} is halo (e.g., —Cl).

44. The compound of any one of claims 1-33, wherein Ring B is a 5-10 membered heteroarylene, which is optionally substituted with 1-6 substituents independently selected from the group consisting of: R^{a} and R^{b}.

45. The compound of any one of claims 1-33 or 44, wherein Ring B is a 9-10 membered bicyclic heteroarylene, which is optionally substituted with 1-3 R^{a}.

46. The compound of any one of claims 1-33 or 44-45, wherein Ring B is 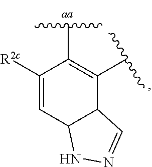
wherein R^{2c} is H or R^{a}; and aa represents the point of attachment to -(L^{A})ₚ-.

47. The compound of claim 46, wherein R^{2c} is halo.

48. The compound of any one of claims 1-33 or 44-47, wherein Ring B is 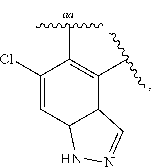
wherein aa represents the point of attachment to -(L^{A})ₚ-.

49. The compound of any one of claims 1-48, wherein Y is —O—.

50. The compound of any one of claims 1-49, wherein n is 1.

51. The compound of any one of claims 1-50, wherein Y² is a straight-chain C₁₋₃ alkylene optionally substituted with 1-3 R^{Y}.

52. The compound of any one of claims 1-51, wherein Y² is —CH₂—.

53. The compound of any one of claims 1-51, wherein Y² is

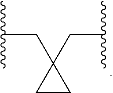 [SMILES: *CC1(C*)CC1]

54. The compound of any one of claims 1-53, wherein R³ is a 4-10 membered heterocyclyl optionally substituted with 1-6 substituents independently selected from the group consisting of: R^{a} and R^{b}; or
wherein R³ is a monocyclic 4-6 membered heterocyclyl optionally substituted with 1-3 R^{a}, wherein the heterocyclyl includes at least one ring nitrogen atom.

55. The compound of any one of claims 1-54, wherein R³ is 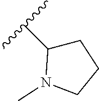 [SMILES: *C1CCCN1C]
optionally substituted with 1-3 R^{a} on one or more ring carbon atoms; or
wherein R³ is 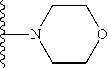 [SMILES: *N1CCOCC1]
optionally substituted with 1-3 R^{a}; or
wherein R³ is a bicyclic 7-10 membered heterocyclyl optionally substituted with 1-6 R^{a}; or
wherein R³ is 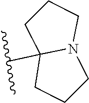 [SMILES: *C12CCCN1CCC2]
optionally substituted with 1-3 R^{a}; or
wherein R³ is selected from the group consisting of: 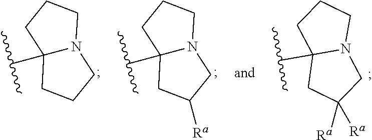
or
wherein R³ is 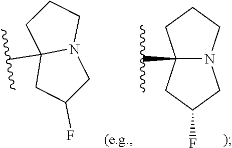 [SMILES: *C12CCCN1CC(F)C2 ; *[C@@]12CCCN1C[C@H](F)C2]
or
wherein R³ is selected from the group consisting of: 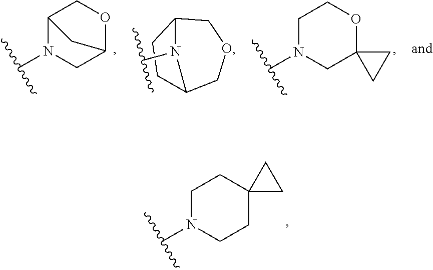
each of which is optionally substituted with 1-3 R^{a}.

56. The compound of any one of claims 1-55, wherein Y¹ is —O—; n is 1; Y² is —CH₂—; and R³ is 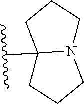 [SMILES: *C12CCCN1CCC2]
optionally substituted with 1-3 R^{a}.

57. The compound of claim 56, wherein R³ is selected from the group consisting of:

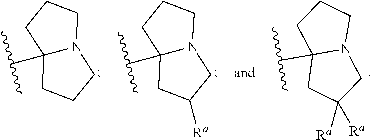

58. The compound of claim 56 or 57 wherein R³ is

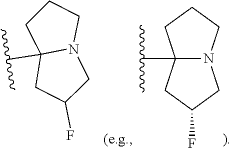 [SMILES: *C12CCCN1CC(F)C2 ; *C12CCCN1C[C@H](F)C2]

59. The compound of any one of claims 1-48, wherein Y¹ is —O—; n is 1; Y² is 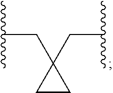 [SMILES: *CC1(C*)CC1]
and R³ is a 5-8 membered heterocyclyl optionally substituted with 1-3 R^{a}.

60. The compound of claim 59, wherein R³ is selected from the group consisting of: 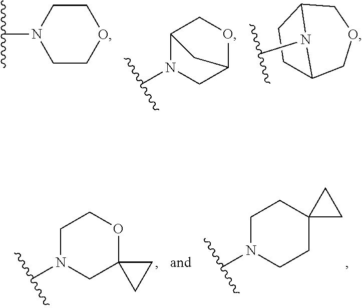
each of which is optionally substituted with 1-3 R^{a}.

61. The compound of any one of claims 1-60, wherein R⁴ is H.

62. The compound of any one of claims 1-61, wherein R⁵ is —F.

63. The compound of any one of claims 1-60, wherein R⁴ is H; and R⁵ is —F.

64. The compound of any one of claim 1 or 3-63, wherein E¹ is CH or CR⁶, wherein R⁶ is halo (e.g., —F, or —Cl).

65. The compound of claim 1, wherein the compound is a compound of Formula (Aa): 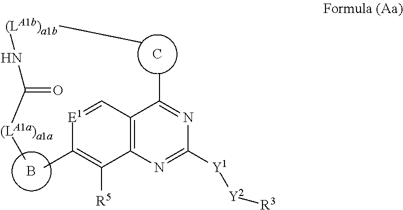
or a pharmaceutically acceptable salt thereof, wherein:
Ring C is a 4-10 membered heterocyclylene optionally substituted with 1-4 R⁷, wherein Ring C has one ring nitrogen atom, 0-1 ring oxygen atom, and no additional ring heteroatoms;
Ring B is selected from the group consisting of: C₆₋₁₀ arylene and 5-10 membered heteroarylene, each of which is optionally substituted with 1-3 substituents independently selected from the group consisting of: R^{a} and R^{b};
a1a and a1b are independently integers from 0 to 5, provided that a1a+a1b is 4, 5, or 6;
each L^{A1a} and L^{A1b} is an independently selected L^{A1};
R³ is a 5-8 membered heterocyclyl optionally substituted with 1-3 R^{a}; and
R⁵ is H or halo.

66. The compound of claim 65, wherein:
E¹ is N, CH, or C-halo;
Ring C is selected from the group consisting of: 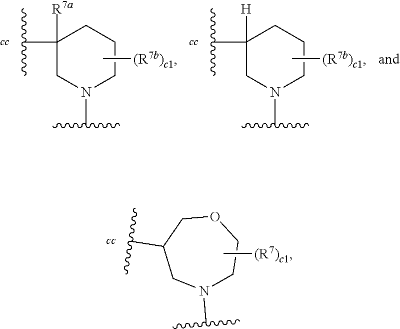
wherein:
c1 is 0, 1, or 2;
cc represents the point of attachment to -(L^{A1b})_{a1b}-;
each R^{7a} and R^{7b} is an independently selected R⁷; and
Ring B is selected from the group consisting of: 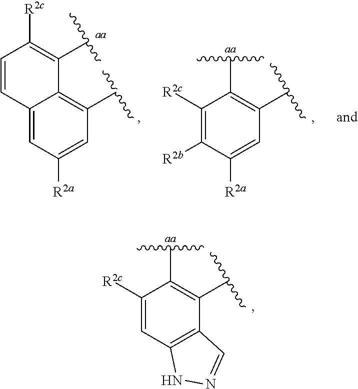
wherein R^{2a} is —OH or —NH₂; R^{2b} and R^{2c} are independently H or halo (e.g., —Cl); and aa represents the point of attachment to -(L^{A1a})ₐ₁ₐ-.

67. The compound of claim 1 or 2, wherein the compound is a compound of Formula (I-c) or (I-cc): 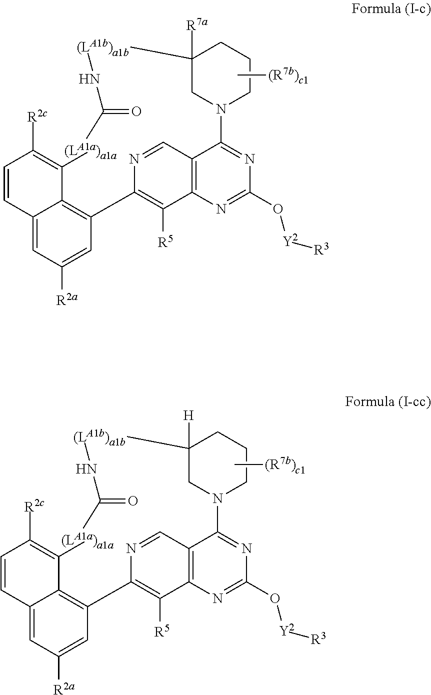
or a pharmaceutically acceptable salt thereof, wherein:
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2a} and R^{2c} are independently H or R^{a};
Y² is —CH₂— or 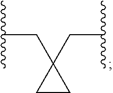 [SMILES: *CC1(C*)CC1]
R³ is a 5-8 membered heterocyclyl optionally substituted with 1-3 R^{a}; and
R⁵ is H or halo.

68. The compound of claim 1 or 3, wherein the compound is a compound of Formula (II-c) or (II-cc): 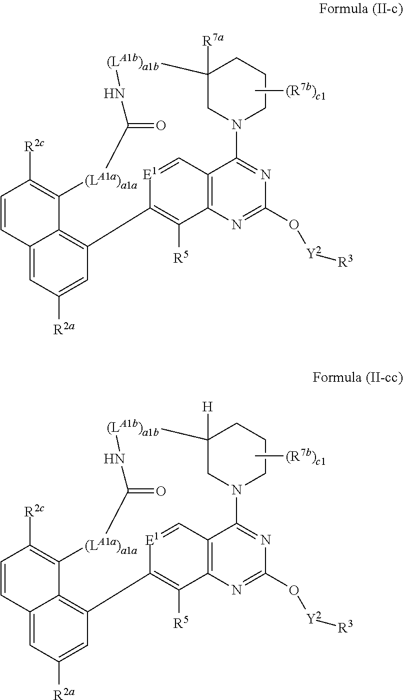
or a pharmaceutically acceptable salt thereof, wherein:
E1 is CH or CR⁶;
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2a} and R^{2c} are independently H or R^{a};
Y² is —CH₂— or 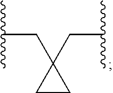 [SMILES: *CC1(C*)CC1]
R³ is a 5-8 membered heterocyclyl optionally substituted with 1-3 R^{a}; and
R⁵ is H or halo.

69. The compound of claim 1 or 2, wherein the compound is a compound of Formula (I-d) or (I-dd): 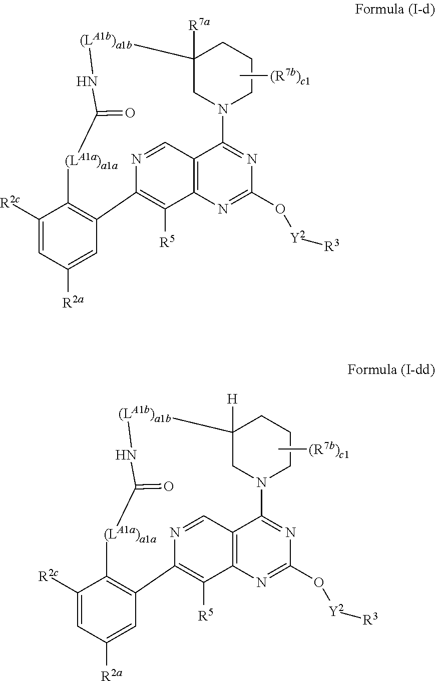
or a pharmaceutically acceptable salt thereof, wherein:
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2a} and R^{2c} are independently R^{a};
Y² is —CH₂— or 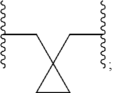 [SMILES: *CC1(C*)CC1]
R³ is a 5-8 membered heterocyclyl optionally substituted with 1-3 R^{a}; and
R⁵ is H or halo.

70. The compound of claim 1 or 3, wherein the compound is a compound of Formula (II-d) or (II-dd): 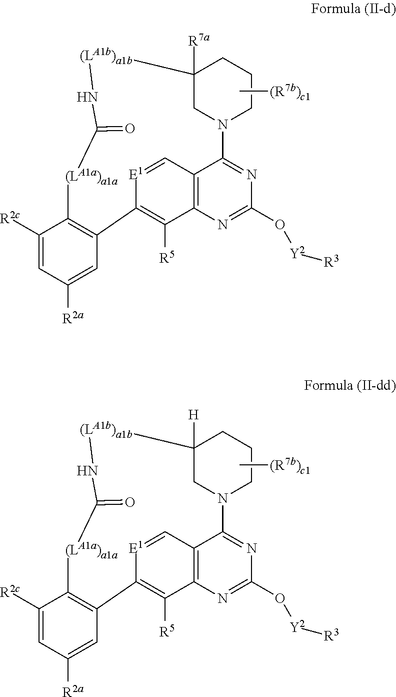
or a pharmaceutically acceptable salt thereof, wherein:
E¹ is CH or CR⁶;
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2a} and R^{2c} are independently R^{a};
Y² is —CH₂— or 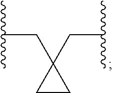 [SMILES: *CC1(C*)CC1]
R³ is a 5-8 membered heterocyclyl optionally substituted with 1-3 R^{a}; and
R⁵ is H or halo.

71. The compound of claim 1 or 2, wherein the compound is a compound of Formula (I-e) or (I-ee): 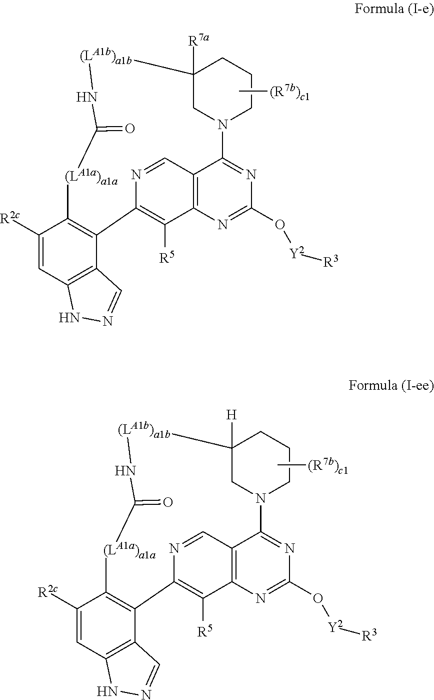
or a pharmaceutically acceptable salt thereof, wherein:
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2c} is H or R^{a};
Y² is —CH₂— or 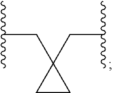 [SMILES: *CC1(C*)CC1]
R³ is a 5-8 membered heterocyclyl optionally substituted with 1-3 R^{a}; and
R⁵ is H or halo.

72. The compound of claim 1 or 3, wherein the compound is a compound of Formula (II-e) or (II-ee): 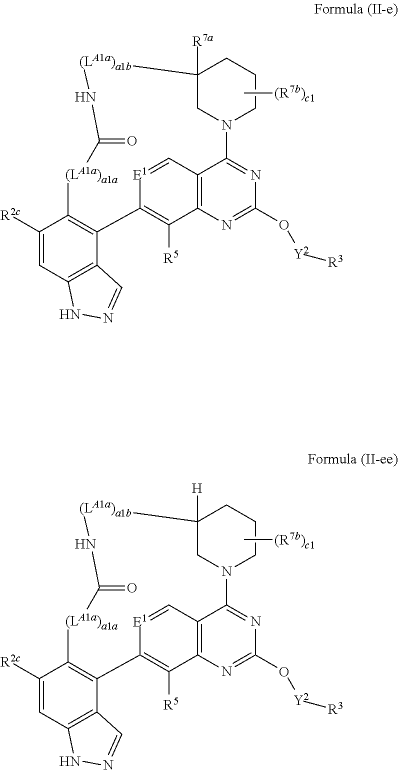
or a pharmaceutically acceptable salt thereof, wherein:
E¹ is CH or CR⁶;
c1 is 0, 1, or 2;
each R^{7a} and R^{7b} is an independently selected R⁷;
a1a and a1b are independently integers from 0 to 5, provided that a1a+a1b is 4, 5, or 6;
each L^{A1a} and L^{A1b} is an independently selected L^{A1};
R^{2c} is H or R^{a};
Y² is —CH₂— or 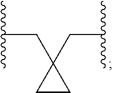 [SMILES: *CC1(C*)CC1]
R³ is a 5-8 membered heterocyclyl optionally substituted with 1-3 R^{a}; and
R⁵ is H or halo.

73. The compound of claim 1 or 2, wherein the compound is a compound of Formula (I-f): 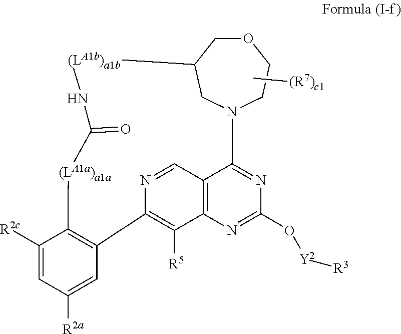
or a pharmaceutically acceptable salt thereof, wherein:
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2a} and R^{2c} are independently R^{a};
Y² is —CH₂— or 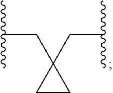 [SMILES: *CC1(C*)CC1]
R³ is a 5-8 membered heterocyclyl optionally substituted with 1-3 R^{a}; and
R⁵ is H or halo.

74. The compound of claim 1 or 3, wherein the compound is a compound of Formula (II-f): 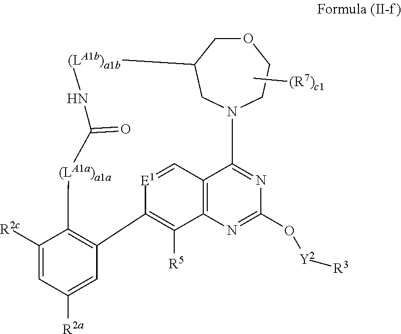
or a pharmaceutically acceptable salt thereof, wherein:
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2a} and R^{2c} are independently R^{a};
Y² is —CH₂— or 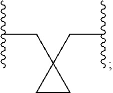 [SMILES: *CC1(C*)CC1]
R³ is a 5-8 membered heterocyclyl optionally substituted with 1-3 R^{a}; and
R⁵ is H or halo.

75. The compound of claim 1 or 2, wherein the compound is a compound of Formula (I-g): 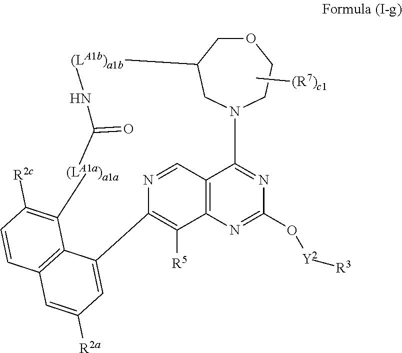
or a pharmaceutically acceptable salt thereof, wherein:
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2a} and R^{2c} are independently H or R^{a};
Y² is —CH₂— or 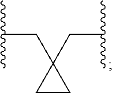 [SMILES: *CC1(C*)CC1]
R³ is a 5-8 membered heterocyclyl optionally substituted with 1-3 R^{a}; and
R⁵ is H or halo.

76. The compound of claim 1 or 3, wherein the compound is a compound of Formula (II-g): 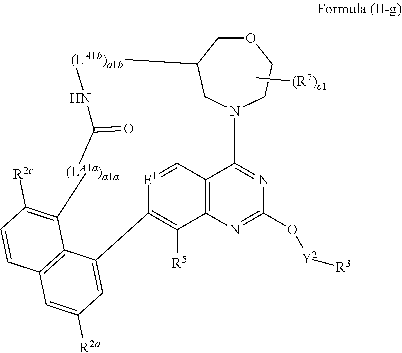
or a pharmaceutically acceptable salt thereof, wherein:
E1 is CH or CR⁶;
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2a} and R^{2c} are independently H or R^{a};
Y² is —CH₂— or 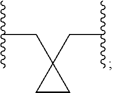 [SMILES: *CC1(C*)CC1]
R³ is a 5-8 membered heterocyclyl optionally substituted with 1-3 R^{a}; and
R⁵ is H or halo.

77. The compound of claim 1 or 2, wherein the compound is a compound of Formula (I-h): 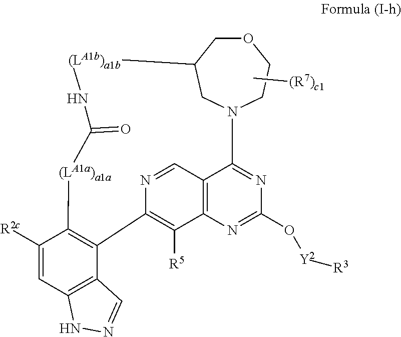
or a pharmaceutically acceptable salt thereof, wherein:
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2c} is H or R^{a};
Y² is —CH₂— or 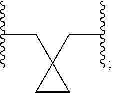 [SMILES: *CC1(C*)CC1]
R³ is a 5-8 membered heterocyclyl optionally substituted with 1-3 R^{a}; and
R⁵ is H or halo.

78. The compound of claim 1 or 3, wherein the compound is a compound of Formula (II-h): 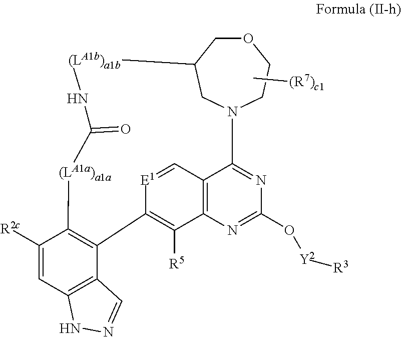
or a pharmaceutically acceptable salt thereof, wherein:
E¹ is CH or CR⁶;
c1 is 0, 1, or 2;
a1a and a1b are independently integers from 0 to 5, provided that a1a+a1b is 4, 5, or 6;
each L^{A1a} and L^{A1b} is an independently selected L^{A1};
R^{2c} is H or R^{a};
Y² is —CH₂— or 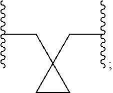 [SMILES: *CC1(C*)CC1]
R³ is a 5-8 membered heterocyclyl optionally substituted with 1-3 R^{a}; and
R⁵ is H or halo.

79. The compound of claim 1, wherein the compound is selected from the group consisting of the compounds in Table C1, or a pharmaceutically acceptable salt thereof.

80. A pharmaceutical composition comprising a compound of any one of claims 1-79, or a pharmaceutically acceptable salt thereof, and a pharmaceutically acceptable excipient.

81. A method for treating a KRas-associated cancer in a subject in need thereof, the method comprising administering to the subject a therapeutically effective amount of a compound of any one of claims 1-79, or a pharmaceutically acceptable salt thereof, or a pharmaceutical composition according to claim 80.

82. A method for treating a KRas-associated cancer in a subject in need thereof, the method comprising (a) determining that the cancer in the subject has a KRas dysregulation; and (b) administering to the subject a therapeutically effective amount of a compound of any one of claims 1-79, or a pharmaceutically acceptable salt thereof, or a pharmaceutical composition according to claim 80.

83. A method of treating a KRas-associated cancer in a subject, the method administering to a subject identified or diagnosed as having a cancer having a KRas dysregulation a therapeutically effective amount of a compound of any one of claims 1-79 or a pharmaceutically acceptable salt thereof, or a pharmaceutical composition according to claim 80.

84. A method of treating a KRas-associated cancer in a subject, the method comprising:
(a) determining that the cancer in the subject has a KRas dysregulation; and
(b) administering to the subject a therapeutically effective amount of a compound of any one of claims 1-79 or a pharmaceutically acceptable salt thereof, or a pharmaceutical composition according to claim 80.

85. The method of any one of claims 81-84, wherein the KRas-associated cancer is a mutant KRas-associated cancer.

86. The method of claim 85, wherein the mutant KRas-associated cancer is a KRas G12A-associated cancer, a KRas G12C-associated cancer, a KRas G12D-associated cancer, a KRas G12R-associated cancer, a KRas G12S-associated cancer, or a KRas G12V-associated cancer; or
wherein the mutant KRas-associated cancer is a KRas G12D-associated cancer or a KRas G12V-associated cancer; or
wherein the mutant KRas-associated cancer is a KRas G12D-associated cancer; or
wherein the mutant KRas-associated cancer is a KRas G12R-associated cancer; or
wherein the mutant KRas-associated cancer is a KRas G12V-associated cancer.

87. The method of any one of claim 82 or 84, wherein the step of determining that the cancer in the subject has a KRas dysregulation includes performing an assay to detect the KRas dysregulation (e.g., a KRas mutation) in a tumor sample from the subject.

88. The method of claim 87, wherein detecting the KRas dysregulation includes detecting a KRAS gene having a mutation corresponding to a substitution of glycine 12 in a KRas protein and/or a KRas protein having a substitution of glycine 12.

89. The method of claim 88, wherein the substitution of glycine 12 is a substitution to alanine, cysteine, aspartic acid, arginine, serine, or valine; or
wherein the substitution of glycine 12 is a substitution to aspartic acid; or
wherein the substitution of glycine 12 is a substitution to arginine; or
wherein the substitution of glycine 12 is a substitution to valine.

90. The method of any one of claims 81-89, wherein the KRas-associated cancer is selected from the group consisting of: a hematological cancer, a soft tissue cancer, bile duct cancer, bladder cancer, brain cancer, breast cancer, cervical cancer, colon cancer, endometrial cancer, esophageal cancer, kidney cancer, liver cancer, lung cancer, mucinous carcinoma, ovarian cancer, pancreatic cancer, prostate cancer, rectal cancer, skin cancer, stomach cancer, testicular cancer, thymus cancer, thyroid cancer, urothelial cancer, uterine cancer, and a combination thereof, or
wherein the KRas-associated cancer is pancreatic cancer.

91. A compound of Formula (SI-Aa): 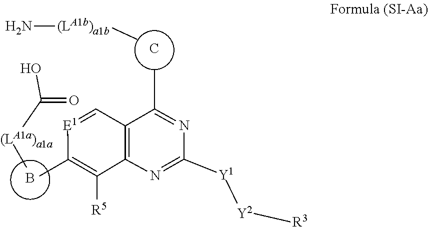
or salts thereof, wherein:
Ring C is a 4-10 membered heterocyclylene optionally substituted with 1-4 R⁷, wherein Ring C has one ring nitrogen atom, 0-1 ring oxygen atom, and no additional ring heteroatoms, wherein:
each R⁷ is independently selected from the group consisting of R^{a} and R^{b};
Ring B is selected from the group consisting of: C₆₋₁₀ arylene and 5-10 membered heteroarylene, each of which is optionally substituted with 1-3 substituents independently selected from the group consisting of: R^{a} and R^{b};
a1a and a1b are independently integers from 0 to 5, provided that a1a+a1b is 4, 5, or 6;
each L^{A1a} and L^{A1b} is an independently selected L^{A1};
each L^{A1} is independently selected from the group consisting of: —CH₂—, —CHR^{L}—, and —C(R^{L})₂—, wherein
each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, and C₁₋₆ alkyl optionally substituted with 1-6 R^{c};
R³ is:
a 5-8 membered heterocyclyl optionally substituted with 1-3 R^{a}, or
—NR^{d}R^{e} (e.g., —NMe₂); and
Y¹ is selected from the group consisting of: a bond, —N(H)—, —N(C₁₋₃ alkyl)-, —N(C₃₋₆ cycloalkyl)-, —O—, and —S(O)₀₋₂—;
Y² is a straight-chain C₁₋₆ alkylene optionally substituted with 1-6 R^{Y};
each R^{Y} is independently selected from the group consisting of: halo, cyano, —OH, oxo, C₁₋₆ alkoxy, C₁₋₆ haloalkoxy, C₁₋₆ alkyl, and C₁₋₆ haloalkyl, or
a pair of R^{Y} on the same or different carbon atom(s) taken together with the atom(s) connecting them form a C₃₋₆ cycloalkyl ring or 4-6 membered heterocyclyl ring, each of which is optionally substituted with 1-3 independently selected C₁₋₃ alkyl;
E¹ is N, CH, or CR⁶;
R⁵ is H or halo;
R⁶ are independently selected from the group consisting of:
(a) —H;
(b) halo;
(c) cyano;
(d) C₁₋₆ alkyl;
(e) C₁₋₆ haloalkyl;
(f) C₁₋₆ alkoxy;
(g) C₁₋₆ haloalkoxy;
(h) —(C₀₋₃ alkylene)-C₃₋₆ cycloalkyl, wherein the C₃₋₆ cycloalkyl portion is optionally substituted with 1-3 R^{g}; and
(i) —O—(C₀₋₃ alkylene)-C₃₋₆ cycloalkyl, wherein the C₃₋₆ cycloalkyl portion is optionally substituted with 1-3 R^{g};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
b is 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, cyano, —OH, —C₁₋₆ alkoxy, —C₁₋₆ haloalkoxy, —NH₂, —N(H)(C₁₋₃ alkyl), and —N(C₁₋₃ alkyl)₂; or
a compound of Formula (SII-Aa): 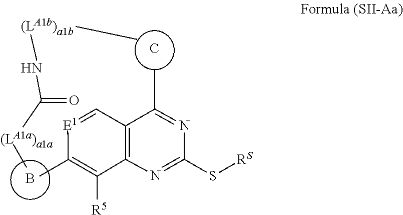
or salts thereof, wherein:
R^{S} is C₁₋₆ alkyl optionally substituted with 1-3 R^{c};
Ring C is a 4-10 membered heterocyclylene optionally substituted with 1-4 R⁷, wherein Ring C has one ring nitrogen atom, 0-1 ring oxygen atom, and no additional ring heteroatoms, wherein:
each R⁷ is independently selected from the group consisting of R^{a} and R^{b};
Ring B is selected from the group consisting of: C₆₋₁₀ arylene and 5-10 membered heteroarylene, each of which is optionally substituted with 1-3 substituents independently selected from the group consisting of: R^{a} and R^{b};
a1a and a1b are independently integers from 0 to 5, provided that a1a+a1b is 4, 5, or 6;
each L^{A1a} and L^{A1b} is an independently selected L^{A1};
each L^{A1} is independently selected from the group consisting of: —CH₂—, —CHR^{L}—, and —C(R^{L})₂—, wherein
each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, and C₁₋₆ alkyl optionally substituted with 1-6 R^{c};
E¹ is N, CH, or CR⁶;
R⁵ is H or halo;
R⁶ is selected from the group consisting of:
(a) —H;
(b) halo;
(c) cyano;
(d) C₁₋₆ alkyl;
(e) C₁₋₆ haloalkyl;
(f) C₁₋₆ alkoxy;
(g) C₁₋₆ haloalkoxy;
(h) —(C₀₋₃ alkylene)-C₃₋₆ cycloalkyl, wherein the C₃₋₆ cycloalkyl portion is optionally substituted with 1-3 R^{g}; and
(i) —O—(C₀₋₃ alkylene)-C₃₋₆ cycloalkyl, wherein the C₃₋₆ cycloalkyl portion is optionally substituted with 1-3 R^{g};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
b is 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, C₁₋₃ alkyl, and C₁₋₃ haloalkyl; and
each R^{h} is independently selected from the group consisting of: halo, cyano, —OH, —C₁₋₆ alkoxy, —C₁₋₆ haloalkoxy, —NH₂, —N(H)(C₁₋₃ alkyl), and —N(C₁₋₃ alkyl)₂.

92. Any of the compounds, compositions, combinations, pharmaceutical compositions, methods, uses, and processes as substantially provided herein.


SEQUENCE LISTING (1 sequence)

SEQ ID NO: 1 - protein, 189 aa, Homo sapiens [sequence omitted]